SEQUENCE LISTING (10 sequences)

SEQ ID NO: 1 - DNA, 281618 bp, Homo sapiens
acaggctccagccgagcggacaggcgtggcggccggagccccagcatccctgcttgaggtccaggagcggagcccgcggccaccgccgcctgatcagcgcgaccccggcccgcgcccgccccgcccggcaagatgctgcccgtgtaccaggaggtgaagcccaacccgctgcaggacgcgaacctctgctcacgcgtgttcttctggtgagtgtccccgcccgaaacaggtcaccctgcagccgctggcgccgcggacgcactctcccggggccgcgcccgatgctgcccaagccctctgcggctgggggcgaggggagcgctttcctacgccacgcggtccacgccggggctttctgctgcgggtggcgccgcccccacgccggccgcgtggtccctgcctgcgccccggggcgcggcgcagggggacgcggggacgcggggccagtggctttgctactggaggccctgcgggcccttagggctcgggggaggaggagcaggaggtggggaggcggggaacccaggctgccgaaagggggagtctcttgcccagcgggagaagcgactgcaccccttacacgcccacccttcgctggctcccgcctcctgctctgggactcgcagtctcctgaactcaaacgcaccttttcctttctttttaagtaagcagcttacgaacagtttcctttatctgatctcagccaaagcctaattagtgtcctggacctatgccatctcccagtagtgtccccaccccttccgatattccttacgagacagctcgatccaactccacgtcccgttgtacctctctgtgtcccggtgtctctgtatcttttgtctcagtgaccagggctctccttttgcctctttgtgggaatctcactgtgtgtccatgtgggtcgtggtacctagttatggcaagtttcatgtgggtaaaataacactattattatttttcatttatgaatggggaccctgttcttgaacaagattttccaggatttgcttttaactgggacatgcaccgccacgactggagtctgtctttgaccagcttctttcccctgtttccaatactttcacttccacgtaggaggacagctcggaggtggggtccctggaatcgctaaccttcagagaggtgatccatggtcttgatgcaggtggttcctaccaggagttctaccttcttaaaaccaccccagggctcctgagctgggagggcccacagccctacccattttgtgcaaggaaacaaacggagaaagactcgggtgacagtagtaactaaaatttccatcttggcctggcgcggtggctcacgcctgtatccctgcactttgggaggcagaggtgggtggatcacgaggtcaggagttcgagaccagcctggctgacatggtgaaaccccgtctctactaaaaatacaaaaattagccgggtgtggtggagcacgcctgtaatcccagctactcaggaggctgaggcaggggaattgcttgaacccagtaggcggaggttgcagtgagctgagatcgccccactgtactccagcctgggtgacagagtgagactccgtcttgggggagtggcgggtgtgtgtgggggaattccgtctgctaactacataagcctgaacctttgaagggcttcaatctcacttggatgattattagatcttctgtttcttgacctctgccgcctcatctttcccacatgttgatattgccttttattatgggacaacctagggtgggggtctccaattttttgggttccctgggccacactggaagaattgtcttgggccatacataaaatacactaacactaatcatacactaacactaatgatagctgatgagctaaaaaaaaatcgcaaaaaaaaatctgatgttttaagaaagcttacgaatttggcagcccatgggcctcaggttggacaagcttgacctaggggattccaagaggatgagtcgcttactcttgtttcgattgtggtacactcagtgggtgtaggaacttctcttggttgtttggcggcctttctcttaaggagaggggcacgtggaggaggaaggaatatgggtgtggtttctgcaaataataacttgaggtagacaaatggtctcctcaacttcaggcagggcaaggaactttttttttttttttttgagacaggatcttgctttgtgcaagctgtgacccaggctggagggcagtgacacattcatagcttgctgcagcctccaactcctgggccgaagcaatcctcctgcctcagcctcccgagtagctggggctccaggtgtgtgtcaccatgcccggataataaaaaaaaattttttttgtagagatagggctctctatgttgttgctcaggctggtcttgaactcctgggctcaagtgttcctcccgcttcggccacccaaagtctgggattacagatgtgactcctgcctccttatatttttctccccctgcattttcaaaatgtaggtggcagggaaataggaagtgatgcgctcccattgatgacttgagtaggtgttcatttccggtttccctgatggaagggaaggagctagtagacctctgttttactccgaggacacttaagctgagagtggttaagttgcctggactcacacaacaatcaagtgtcagagcaggctccaaagctgggcagactggctccccagcctctttggagctgctgtactgagctgcttccagttcaggtgctgtcggaaatgcatggagaaggcgctccctgttctggaaggctagagtttctcaagggaaattcttaaagtacctgcagttggagatgatgcatgtgtaacaactattagtgtgctttagggcagctgttctcaaagcatagcactcagagcagcagcatcggcatcacttgggagattctaagagatgcagattctccagcatccactctggactgattcagtaaaactggaaacactgggtgtgtgggtgggggtggtgaggtttcaagtgctccaggtgatcctgatttgaggcgcttactgcttccagctactcctttcattggagtattattacagtattatgcttttctttatttttaattgttaaaatttctttatttcttttttttgagacggagtctttctctgttgctcagctctgtcacccaggctggagtgcagtggtgcaatctctgctcactgcaacctctgcctcctgggttcaagtgattctcctgccccagtctctcaagtagctgggactataggcgtgcaccctcacacccagctaatttttgtatttttggtagagatggggttgcaccatgttggccaggctggtcttgaactcctgacctcaggtgatctgcccgccttggcctccaaaaatgctgggattacaggcatgagccactgtgcctgggccagtttgagggattttaaagtctgaggcctcatagtgagaaaataataataatcatcatctgaggaatgcggagtccttttaaattatcaggcccagagagacattaaaatgacacagcagtcacctcgtagttcccacccccttgagctatttacttatcactcgaaagtacttcatattgccacatagctacagattaacctaacagtgccccaaggacactgtaatccacagctcaggagtttgagctccagcgatctgcctgcctcggcctcccaaagtgctgggattacaggcatgaaccaccacactgggtctgtagtaaattcttgagttatattttgtgccttagcctcctgcttttaggtcagtatttgtgtacccttcccactgccagtggaaagctgaaatgagcccatccaggaagtggctgcctggagacagggcccaccttgtagggaaaaactgctttttcaccttcttgttgagtgtttgcttttcttctcaaatatcatctgctttctgaacttgtgaactggcagctgcctacctaaatgatgccaataactggcttactgacttttattcaaagtattttcccgggattttggtgtatttatagcataatttggacttggagtctaaaaggagtcatgcattatggctgttggcaattgtggatttttaaaggtagtattttcacctatttctttcttttttctttcttttttttttttatgagacagagttttagctctgttgccaggctggccgtggtgtgatctcggctcactgcaacctccgcctccagggttcaagtgattctcctgcttcagactcccaagtagctgggattacaagtgcccaccaccatacctggctaattttcatgtttttagtagagacagggtttcaccatgttggccaggctggtctcgaactcctgacctcaggtgatacacctgcatcggcctcccaaagtgctgggattacaggtgtgagccactgtgcctggccttccattttcttttagctattgtttcatttctaacaataaaggcagctagaatttcttttggtatttgatatgagatagtccccattttttccccaaacagctcgttttccccattatcatttattgaataattcctcctctttcccgttagtggaaataagtggcatagcctatgaactgtggccagctgggcacaagatgggtcaatccctgagactctcctaggaattgtcctgctgccccctggtgcccggcaggcactttgcattcctctgcccacacctgatccagaggtgatctggggtcagcagcagggtggaggctaacaacgaaggacttttccctaaatatgggcattgagagggatctccagatgcatctggcccacagaaagtggttaaatccttgctcccctgttgctggccatatctcagtgggtgaagtgtcatcttgcaaagcctgtgctgctgtggaaactgccactggtgaccagacagactctctggagacctgccctagagagctgatgacatgactatagaagcaaacacaagtggctgagggcaggggatggcttagagaggaagggtgtgtgaggccctcagtcaatgcatcaaatgtttaattttatattttctttttctataataaaaaaaatatttttcttgtagagatgaggtctcactatgttgcccaggctggtctcaaaatcctgggctcaagcagtcctcctgccttggttttcccaagcgttgggattacaggaatgagccactattcctggccatttaattttgtgtttttgtgtgtgtgtgtgtgtgtgtgtgtgtgtgtgtgtgtgtgtgtgtgtgttttgagacagggtcttgctctgttgcccaggtgggagtgcagtgatgcaatcatggctcattgtagcctcaaattcctaggcttaagtgatccttccagctcagcctcccaagtagctgagctacaggtgcacgccaccacacctagctaaagtgttttttttttttttttttgacggagtctcactgtcacccaggctggagtgcagtggtgtgatctcagctcactgcaacctctgcctcctgggttcaagtgattctcctgcctcagcctcccaagtagctgggactacaggtgcctgccactatgcccaactaatttttttgtgtgtttttagtagaaatggggtttcagtatgttggccaggctggtcttgaactcctgaccttgtgatccgccctccttggcctcccaaagtgctgggattacaagcatgagccaccgcacctggacacctagctaattttttgaggggcgggggacggagtcttgctctgttgcccagtctggagtgcggtggcgtgatcttgggtcactacaatctctgcctccagggttcaagtgattctcctgtctcagcctcctgagtagctgggactacaagcgcacgccactgcatccagctaatttttgtattttcagtagagatgggatttcatcatgttgaccaggatggtcttgatcttctgaccttgtgatctacccacctcggcctcccaaagtgctgggattacgggggcgagccactgtgcctggccagtaatttttattaatacatttttggtagagaccaggtctcgctgtgttgcccatgttggtcttaaactcttggcttcaagtgattctcccgccttggcctcccaaagtgctgacattgcaggtgtgagtcactgtggccggccaaatagtaaatttctgtgtctgcagaatcacagtctcttggggttgagcatcctgaaatctgcattttttaaagcattatttcctggttgattcagatgctgagatttgggagccacgtttctcaggtctttctcagtctttggtgtccaattctacttgatcggtgtctgggaggccaaggttactgccttgatcctctgcggtttagattattcatttagcaaacatttgagtgccttatttttattttattttttgtttttaattttttaatttttgagatggagtttcactcttgttgcccaagctggagtgcagtggcgtgatctcggctcattgcaacctctgcctccggagttcaagcgattctcctgtctcagcctcccaaatagctgggactacagatgtgcgccaccatgctcagctaattttttgtacttttaggagagatggggtttcaacatgttgtccaggctggtcttgaactcctgacctcgggtgatccacccacctgggcctcccaaagtgctgggattacaggcgtgagccgccatgcccagcctggactatgttttctaccccaaccttgtggtcagaggtgaccatgaaactgagacctggccaataggaagtggttgcaaaaggtgtctcgcttccacgactggaacacaaaaaccagccccatgcaacgcctctccagctgaataaagaggcctcctggagaggaggaggactgagaggaggccgagccgcaagatggtgaaagtccagctaggagtaaatgtatggtgttgttcaccaccctagatggcccaaaaggggactatagcatgacaataggcatggtcttggtttcttgttttcttttttgagacggagtctatctctgtcttgcaggctagagtgcagtggcgtgatctcggctcactgcaacctctgcctcccgggttcaagcgattctcttgccttagcctcctcccaagtatctgggattacaggagctcaaaaccacgcccggataattttttatttatttatttattttgtatttttagtagagaaggggttttgctgtgttggccaggctggtctccaactcctgacttcatgtgatccactgcctcggctggtgattttgaattaagccattgagaatcagggcttgtttaatagctagcattatttgcacgatacagtggctgcctccctcattgtgtgagctctttgagaacaggaatggtactttattgtaatggatttgcacctggcaagaaagaaggcaccctgccttctttcatggtggcacatgcctgtaatcccagctacttgggagaccgagcaggagaatcgctttaacccaggaggtggaggctgcagtgagccaagattgcaccactgcactccagcctgggctacagaatgagactctgtctcaaaaaaaaaaaaaagcaaaagaaaacgtagtccaggctgggtgcggtggcttatgcctgtaatcccagcactttgggaggccgaggcaggtggatcacaaggtcaggagttcaagaccagcctggccaacgtaatgaaaccccgtctctactaaaaatacaaaaattagctgggcgtggtggcatgcacctgtagtcccagctactcgagaggctgaggcaggagaattgcttgaacccgggaggcagaagttgcagtgagccaagatcacgccactgcactccagcctgggcaacagagcgagactccatctcaaaaaaacatagtccgtgtgtccagcagtcgctgctgcaggagttcatcctgaacccgtgttgggtgtgctggagtagaagtatggaaaagctgtgtgagggtggggattttgtttttgtttttgttttttgagatggagtctctgtcgcacaggctggagtgcagtggcatgatctcggctcactacaacctccgcctcccgggctcaagcgattctcctgccgcagcctcccaagtagctgggattacagatgtgcaccaccaggcctggctaatttttttgttgttgttagagacagggttttgccatgttggccaagatggagatctctggggaggggtttaccttcctgttggagcttggatggagaaggtgggaaatcgttcctggcaggggatgagatgtccaggtcacagaactgcagttggagaaagtgttgcctgagacgtgtggcgaatgaccacccctcagtcgctatgtcgtccctgccagccggtagcgtttgttacacccaaggttgccctcccctctttcatcccagcagatgttcagactttgcttctcccacttccagccccttactcatgagtacttacagcccttttgtggcctgcgacgtagctccttgttccatttctgggttccatgaccacttccgtgctggtctgtttcaggcctagtccctgtctgctcttttctctgcgctagctcctatcttccccttttccttagtgtcatagaattagtgggttgaaaggaagagatcttctagcccacactcagtacaggaatcccttccagatactgggcagtggggaaatgcattatgtgactctcatgactttagattcgaatgccagaattatgttgtattgagccacagttttctttccatgcaatttctacttgtgtttacttacttctaagaaaagcattacagaatttaatgaagcaaagaatgatctgtgaatcaagcagctcctagaaccaaaataggttcagagccgctctcctttgtttttttgtcaacacgcagagcaagttggctacctcccttcatatgctcgcctcatagtattcaaagacagctatcatggccacattaaatctttgtttctttgtttgagacagggtgtcactctgtcacccaggctggattgcagtggtgcgatctcagttcactgcaactttcacctcctgggctcaagccatccttccacctcagccccctgagtagctgggaccacaggtgtgtgccagcatgcccagctaattttttatattttttgtagagatgaagttttgccatgttgccctggctggtattgaactcctgggctcaagtgatcctcccgccctagcctccaaaagtgctgggattacaggcatgcgccatggtgcctggacaggagtttatttttgaccctgttaagtttgagatgcctgctacacattcaagtggagatgttgaataggcattcaattggtcatgagtttggggctcatggagaagtttatcttattattattatttctttaaatggagtttcactgtgttgcccaggctggcgtgcagtggtatgatcttggctcactgcaacctccaccttctggttcaagcaattctcgtgcctcagcctcccgagtagctgggattacaggcacgcgccaccacacccagctaatacttgtattttagtagagatggagtttcatcatgttggccaatgtggtcaggaagtttatgtcaatttgggcatcatcagtgatacatgcagttgagctatattggacacatggccaattgacctcacagtccgcagggcttgtccccatgtaactgctcttctatccctatgcaaattcattttgtagatgctaacgtttagcagctcattgttgctgttgaaagttcttggaacactttcgtagtctgaatgctagaaaggatgggtcctaggaatgtcctctaggttgtggcagatccactaatcaacactgtggttgctagattaacttggccacaagtgtcaagagaggtcgtgtcatatggaggtctgctttatggaagtcaagaggtattggagtattggatcacctgggcacttctgtcttttttttttttttgagacaggatcttgccttgccagccagccttggagtgcagtggtgcattcatagcttattggagccttgacttcccaggctcaagtgatcctactgcttcagccccccaagtagctgggactacaggtgcgggccaccacagctggctaatttttctaattttttgtagaggtggggttttgccatgttgcccagactagtctcgaactcttgagctcaagtgatccacctgccttggcctcccaaaatgctgggatgacaggtgtgaaccgtcatgcccagcctgggcagtcattcagaaaccctaccaagaaaagggttaaagtcatttggcatggtttcctctttttttttttttgagacagggtcttgctctgtcgcccaggccgaagtgcagtggcgcaacctcagctcagtacaacctctgcctcccggggttcaagcggttctcctgtctcagcctcccgagtagctgggattacaggtgcttgccaccacacccggctaatttttgtatttgtattttttatttttatttatttttttgagatggagttttgctctttttgcccaggctggagtgcaatggcgcgatgctactgtgcctggcctcacgatttcctcttttttttttttttttttttttttttgagacagagttttgctcttgttgcccaggctggagtgcaatggcacgatcttggctaactgcaacctctgcctcccaggttcaagtgattctcctgcctcagcctccccagtagctgggattataggcatgtggcactacgcctagctgatttttgtatttttagtagagatagggtttcaccatgttggccatgctagtctcgaacgcctgacctcaggtgatccacctaccttggcctcccaaagtgctgggattacaggcgtgagacaccacacccggccatgattcctcttaacacaagctggctcttggtgctgcttttcatgaatatactctcaaattgctcccttcaaatgattgttgatgtgcattatttcttttaaccttattagctttattttttctaaattctttttagattacatagcagtttcttagccctcaccatgattggatcacctggacagccataaaaatatggatgtctgttccccttataggccaattggaaaaataatttctgggggtgggatccacgcatctatctatctatcatctatctatctatctatctatctatctatctatctatctatctattttttttgagacagagttttgctcttgttgcccaggctggagtggaatggtgtgatcttggctcaccgcaacctctgcctcccgggttcaagcagttctcctgccttagcctcctgagtagctgggattataggcatgcgctaccatgcctggctaattttgtatttttagtagagatggggtttctccatgttggccaggctggtctcgaactctcgacctcaggtgatccgtcagccttggcctcccaaaatgctgggattacaggtgtgagccaccgcgccccgcacatcaatattttaaaaaggttctctacgtttctgatgggcactgaagcccaagaaccactgatcaactgcttgccaaagtcaaaaatatttctgtgcatatttccatttttctggccaaaggttcgctgcccctttttcaggttcctctcaaaggacttgtctgatctcaaagcatgagaaccattaccacaataggacttactcccctctgtgggtgctcagccagtggctgagggagaagggggattgctccgtgccagggatgttgttagagatgccgggttcttcgatttttatagtaaccccttgttgagggtggctctgctcttgtgttacatacaggtgaaggaaactgagagagtccaggtagcataatgagggtcacccaggcaggacatgatggagttgggaactgagtcaagtctgacaaattccaaagcatgagctgcagcctctgcgtcactatgtttattttctcaccctgggtcagagattaattgagctcatgagagtacttgacaggaaatctcacacttgtgaattcagagctcgtgagcataaaattgctcttctaatcacttgcttttatttgctgatctttattggtgcattttattggtcaggatgtgattgagattatgtaataaacccaactgaccgcttcttttaacttctgctattgaagtaatccctcagattactgtcttgagagacggagagtgcatacagtccaacagtgtgtactcttcctctttaaagacagtagaaaggtatccatcttgttttaaatagtagcagggaggtttatcaatggcttaacatggtaggctgggcctggagagctctcaatgtaacagagctcaaatgtggcatttacatgaattctaagttgattaagcgacacaatttgttcaactataaaacataatacattcttaatagattttacctattattgtatatgtaagttcctagtacatattaggctgtaggtaaatgggatgatgagggttcttgctaaatttatcccccatccttttttttttgagtcagagtcttgctcttgagcaacctccaccttctgggttcaagcagttctcctgcctcagcctcccgagtagctggaattacaggcttgtgccatgacacctggctaatttttgtatttttagtagagatgggactttgccatgttggccaggctggtcttgaactcctgacctcaggtgatccgcccacctcagcctcccaaagtgctgggattacaggcatgagccactgcgtccagcctaaatttacccttttatcatgccttttttattgtcttaagtctttaagtcttatttaagtcttgactcatttgcgacttttttactaaattgttagctctattaaggcagaaaccagtatcttcaatgtcttaatctctcagtgactggtttaacatcttgcaattaatatgcttaattaatgtttataagctattactttagtcttaatctcaatttcttgaatttagagttgctgacacctgtgtcttaggtaggaccaagtgagaagctagttattgtgacagcctcagactggagccagaagacccatgatcttgtatatattagctgtgtgtgaccttaggcaagacacatgaccactctgggcttcagtatcctattctatgatttagaaaaattaaacctactcatggcatgttttaagaactatattttaaaaatacattataaaatatttatttatttatttattatttgtgtgaggcagagtctcactccgttgcccaggctggagtgcagtggtgcgatttcggctcactgcaacctccgtctcccgggttcaagtgattctcgtgcctcagcctcccgagtagctgggactataggtgcatgccaccatgcctggctaattttttgtagttttagtagagacggggtttcactgtgttagccaggatggtcttgatctcctaacttcgtgatctgccagccttggcctcccaaagtgctgagattacaggtgtgagccacctcatctggcctatttatttatttatgaggtctttctgtgttgcccaggctagagtgtagtggctattcacaggcacactacatcctcaaatttatggcctcaagaggtcctccttcctgatcctcccaagtatctgggactggctccaagaggtgtattttaaggaaaaagtttttttttttttgagacgaagtcatgcccttccacccaggctggagtgcaatggcgtgatcttggctcactgcaacttctacctcccagattcaaacgattctgctgtctcagcctcctgagtagctgggattacaggtgcctgccaccatgcccagctaatttttgtatttttagtagagacggggtttcaccatgttggccaggctggtcttgaactcctgacctcgtgatctgcccaccttagcctcccaaagtgctgggattagaggcgtgagccaccatccctggccagttctctttttttggagacagagttttgctctgttgcccaggctggagtgcagtggcacaatcttggctcactgcaaccttctgggttcaagtgattctcatgcctcagcctcccaggtagctgaaactataggcatgtgctaccataccgggctaattttttgtattttattagagataggtttcaccatgttgcccaggctggtttcaaactgctgagctcagccaatctgcccaccttggcctcccaaagtgctagagttacaggcgtgagccatcgtgcccggtcaatgaaacaataaagttctatacacttatttttcttttcttttcttttttttttttttttgagacagagtttcactcttgttgcccaggctagagtgcaatggcatggactcagctcactgcaacctctgcctcggggttcaagcaattctcctgcctcagcctcctgagtaactgggattacaggcgtgtgccaccacacctggctaatttttgtatttttagtagacaaggggtttcaccatgttggccaggctggtctcgaactcctgacctcaggtgatccacctgcctcagccttctaaagtcctgggattacaggcgtgagcctccgcgcccggccttatacactaatttttctccctcacctccaatcaagcagggaaagtgaatatggaagtaggtcagtgtataatgtcccatctctggtgaatggagaagtcacctgtttgtaagtccccccagaaatgaagatctattaaaaaaacaaaggccacaaagggaaatttaattcatactcagaagtgggccactatctatggaagtacttggaaaataataaggtgcgttattattacatctttaagaagttttcctgggctgggcactgtggctcatgcctgtaatctcagcactttgggaggccgaggcaggcggatcacctgaggtcaggagttggagatcagcctggccaacatagtggaaccctgactttactaaaaatacaaaaattagctgagtgtggtggtgggcgcctgtaatcccagcttctggggaggctgagacaggagaatcgcttgaacctgggaggcggaggttgcagtgagccgaaattgtgccactgcactccagcctgggtgacagagtgagactccgtctcaaaaaaaaaaaaaagtttttctggaaagcagtttcacagcagagcaacaaggctgaaattgccccagtcagcctttgctggctgacttccctttttccgttaatagcaccaccacacccagtaactaaggcttgcgtcctcagttatctgcacgtagggaagagtctggtcagtgataaaaggtagttggtcagttgccacgtcttgactggaggcacgcaatgattccagattccaacccagtcaatacactgcaggctttatctttacaaggcatagctctgctgctttctgttccagaacctttggtggcatcttctttggcatttaatactctccttttcctggtcctaacttagctttcctacattctcagatatccattgttcctggacagtcaatatatgatgcctttcccctgatcacaccttatatttgctgtcctgttcccttagctcatgttatataccctggctagagtacctacctctcagctcggctgctaaaattctccctgaaaacccatccattaaccaacttctagtccattattcagaggatatggaactgctatttatttatttatttgagatggagtcccgctctgttgcccaggctggagtgtagtagtgtgatcttggctcactgcaacctctgcctcctgggttcaagtgattctcctgcctcagcccccaaaatagctgggattacaggcacccgccaccatgcccagctgattgttgtgttttagtagagatggggtttcaccatgttggtcaggttggtcttgaactcctgtcctcaagtgatcctcccgcctcagcctcccaatgtgctgggattacaggtgtaagctaccacgcctggcccctatttatttatttatttaagagacggagtctcactctgttgcccaggctggagtacagtggtgtgatctcagctcattgcagcctctacctcctgggctcaagtgattctctaatcacagccttttgagtagctgggattgcaggcatgtgccaccatgtctggcttacttttgtttattttttgtagagacgagatctcactatgttatccaggctggtctcaaactcctggactcaaatgattctccttcctcggcctctcattctgctaggaatacagacatgagccaccatgcctggcctggaactactgtatatttagacattataattatgtactgcttcctgtaggcaggggggctattctaagtaccttaccaataaatattattaaatactaataacaaacctttggggtgggtactattatttcccacggtcacacagcctttgagtggcagagcctaggttcaaaccaggtaggtaacttagcacagagtccctgctctcagctgcagcgtcatgtacctcttgattataggtgctgggaatacagtgaccctggtccctgccctaaggggcttatgttccagtgggagtcaaaccatatacatttttgagtctctgctgtttgccaagcacaggggacaggggtgtctgccgaaaaggccgtgtcatctgtgtagctgctagacactgtcagttccctgtgggcagtgtccttgcctgtatatgtcccaagtacctgacctagtcctggcacaaagtggctcagttggctgggcaaggtggctcatgcctataatcccagcactttgggaggctgaggcaggtggatagcttgagcctaggagtttgtgaacagcctgtgcaacatggtgaaactcttgtctctacaaaaaatacaaaaattagccagtcatggtggtgcacacctgtagtaacagttactcaggaaggctgaggtgggaggatcacctgagcccaggaggttgtggctgcagtgacacatgatcgcacccctgcactccagcctggacaacacagtgagaccctgtctcaaaaaaaccccaaaaaacaacaaccaaaatctcaaacaaaaccaaaacaaaaaacaaaaaacctgcctcccaggttcaagtaattctcctgcctcagcctccccagtagctgggattacaggcatacgacacatgccaccacgctcggctaatttttttgtatttttagtagagacggggtttctctatgttggtcaggctggtctcgaactcccgacctcagatgatttgcctgcctcggcctcccaaagtgctgggattacaggcgtgagccactgcacctggcgccactctttttttttttttgagacggagtttcgctcttgttgcccaggctggagtgcaatgacgagatctcggctcacggcaacctctgcctcctgggttcaagtgattctcctgcctcagcctccctagtagctgggattacaggtatgcaccaccacgcccatagagacggggtttctccatgttggctaggctggtcttgaactcctgacctcgggtgatccacccacctcggcctcccaaagtgctgggattagaggtgtgagccactgtgcctggccccctctcttaatctctgcattctgctgccactcagcatgagatgctcttcagaaaaaatatccttctggctgcaatataaagttaggggagggcaggaatggagggaatgagttaggagctagctacagtcctagttgcttgggaggctgagatgggggcattgcttgagcccaggagttcaaggctgcagtgaggtatcattgcaccactgcactccagcctgggagacagcaagaccctgtctcaaaaaaaaaaaaaaaaaaaaaaaaaaaaccacagaaaaacttcaaggtggctctaggattagcagcatcagcagcagcctgtgttttaatcagtcctccaagtgattctgatgcatgctgatgtttgagacccactgccacaggaccagtttttaaacttttttttttttttttgagttggagtcttgctctgttgcccaggcaggagtgcagtggcgctatctcagctcactgcagcctccgcctcccaggttcaagcgattctcctgccccagcctcccaagtagctgggactacaggtgcgtgccaccgcgccaggctaattttttgtatttttagtagagatggggtttcactgtgttgaccaggatggtcttgattcctgaccttgtgatccacctgccttgggatcccaaagtgctgggatacaggcttgagccactgtgcctggccagtttttaaacattttgatcacgttctacaagttttacatatgtaatgaccaagagatatattctctttctctctctcacacataacatatttaacatactttaggctggacacggtggctcacacctgtaatcccagcactttgggaggccaaggtgggcagatcacaaggtcaggagtttgagagcagactggtcaacatggtgaaaccctgtctctactaaaaatacaaaaattagccaggcatgatggcgtgcacctgtaatcccagctactcaggaggctgaggcaggagaattgtttgaacccgggaggcagaggttgcagtgagtcgagattgtgccactgcactccagcctgggcgacagagcaagactccttctcaaaaaaaataaaaaataaaaaaataaaagaaaaacatactttagtaaaagtttcaagaaacagtgtcctgtcttaggaaacatgtgacacactggtattttcgattctgtttcattaaacaaagtgctttgagacctgtcagaacccctgctcttgagcaaatcaagaaggttgctgtgctgcaagggtgcacttccaaagccaccttcagtgggactttcctgtctttgaagaagcctttcacttccagagcttgacccagggagtccttgtttctctaagtctggacctgctcctttatcacccatattacgggctgaattatgtccccccaaagttcgcatgttggagtcctgatctctagtacttcagaatgtgattgtatttgaagacggggattttttttttttttttttaagatggagtctctctctgttgcccaggctggagtgcagtggctctgttgtccaggctggagtacagtggtgtgatctcagcccactgcaacctccacccctcccaggttcaagcaattctcctgcctcagcctgacgagtagctgggactaccggtgcgtgctaccacatccgggtaatttttgtatttttagtagagacggggttttgccatgttggccagcctggtctcgaactcctaacctcaggtgatctgccctccttggcctcccaaagtgctgggattacaggtgtgagccatagtgcctggcctgattgtgattttttttttatggcagcctgagcagactatattacagcccacaagcctcacaaagatattgcctggagttagtgccactggttgtcattttccagagctgctttgtagggcagtaatgtgtcctctgttagactgaactcgccaaggagtttgaccctctattccacctgcttattatagaactactccctcttttcctcatatttcttgggttgagtttgggtaagattactaaaattcttcagatgtggaaccaatcttcataaacacttcagaatactttgcttccttctgaattactacagacaagcagtctgggaggtccaagtctgtataattattgttcttaacattctgtcactcattattgttggtgttcgtttctcaatgccagatgaggtgtgtcttgggcaaaagcctgtccagtgtaagctgttctgttctttcaggcagaatcactcagacttatgctctcaactttctctctctctttttttttttttttttttttttttgagagagtctctgtcgcccaggagtgcagtggtgtgatcttggctcactgcaacgtccacctcccagattcaagtgattcccctgcctcagcctcccaagtagctgggattacaggcttgcaccaccacacccagctaatttttgtatttttagtagagatggggtttcaccatgttgccacagtgcctggcctgaactttctcttttacagccacatgcaaattacctgaactatctggcttttgggaaaaccctgtgcaagttcttagcatccagtattaaaagaaaagagatggctctgttctcttagcaactcaaaatcctcttccttctccccctttaaaatataatgtagtccttgtttttccttaaaataaatattttaaagagaaaattggtccatttgaagaagccaaaataggatctcttcccctctccttgttgctgtctctctcgagtggattttggtcatttctgctaagtttgcccagaaaataggctgaagtctgtggaagggaaattccacaaagctgcctcatgtccacagttcctcgggaggaattcctttcctgtaaggaggaggcacattttctgagagtcccttccaggaaggatgaggtgcagcaggaaggaatcccacagagagccgtctcacagacacagaaaagggaaggaattgcctttgacttctgggaattggatggttttttttttttttttttttttaatagaaggcaaaacagcaaatggcagctactagtgactgactatatcaggttttggaaaagtgggcagtgagggttttgttgattggattgaactctgcttggcaaaccatagatgaccctgagtaagccttaggccttttttttttttttttttgagatggagtctcactctgtcacccaggctggagtgcagtgtcatgatcttggcgtaccacaacctctgcctcctgggttcaagtgatcctcctacctcagcctcctgagtagctgggattacaggcacctgccaccacacctggttaattttttgtattgttagtagagatggggtttcactatgttggtcagggtggtctcgaactcctgattacaagtgattcacctgccttggcctcccgagccttagcccttttgattccctgacatcaaatgctacttctcttggatagttatactgtgagaagttggtgacgggcaaaacgggagcccaggccagtgtcagaaactcacccagagtcgcagggacaaagctgttgtgtgggttcttccctgtgagaatcttaaggatggaaatggtgagcacaaaccttggcatttaaggaccgacaatgctgagatgttgtggtgggtccttgagtagggctcaagtgtccttccagggtggtggtcccagggaattgggccagaaacaaggccacccagtctcagctctgttgttcctggggggccctgtggctccaggagctcgtgaacatggcgaaccccacttccccatcccctttctgccttctcttctgttctcttgacagtctctgttttctacccactggggtcagcaaatcagccccttttaccggtttccacaaaaatccacaaaaagcactgtgaggacttggggatttcttgaagatttgggttgaggtatttcaggagggccttgcctcgcccctttccactccatcttcctctctgctccccacttctctggaataagaggtactgccctccttatttcagcgctgtcattctgatcactctaggaccggttgagactttttcgtgaacagtttcctacctttttaggcaaccatctgtaagacaaggtagaatgctaccataaccatgattaacatttgcaaacactctgccacagtgcagccactttctttgtctccagctgaagaaattgaggtgcacagaggttaaatttgatgctcaaagcaatacaactagaaaatataggaggctgggctgcctgattgattcccagcttagagattgaaagtcacatgcgaagcgctttagcagatagagaagggggagtcatactccagtgtgcattttagtttgtgcttttctgtccttgaattaagaagtgtgacgtgtgcctccaacagcatagcatgctacagaaatggatttcttttcctggggttgataatctgctggccaaatctttatctctgtagtatcctgtgtttcctggtgcattttagcatttggttaaattgtagggaaaagggtttgtgttctttatatctggattgtctattagatcacgtttttttgtgttgttatttgcgtctttttttttcctggaaggaagtgattggctgtaacagtttctctcccactccggtctctgaggaaggaaaaaaaaaataccttggtttcatatgaaattagactaatagaatttctaattatctgagttcattgagttgttgtcagtctgtgtttttttttttttttttttttttttttccgagacagggtcttgctctgtctctcaggctggagtataagtggtgcgatcatagctcactgcagcctcaacctccctgtgctcgggctcaggtgatcctcccacctcagcttcctgagtagctgggactataggcatgcaccaccatgcctggctaatttttgtttttgttttgtagaggcagggttttaccatgttgcacaggctgctttcaaactccctggctcatgcaatccacccacctcagcctcccaatgtgttgggattataggcgtgagccaccgccccttgcctctctgtctgggcttggtcctcctgggaacctctgccttgatcacccatctagaagaatggaggacctctgccgtttcagtcatccttttcacctgtaccagttggcccaggagaatcctggtttatatcccttatcacactgccactattaagctccccctttcactcctaagagtatcccaggttagatgatatgaccatgaatggagctgaagttcatattatacataatatgaagtatacataaggtatacgggccctctaaagccatggggcataagcacaaactgtgggcacagtgatgtaaatatttgctaaacttttatccaaagtaatgattggtaaaaacctggtagcatctccacttgagcagacatcttcagaaataacctgtaacctgtgctttatacataagaacaatggcatgcggcgttctctcctccgtccctgggttgaccgcggtggggtagggggtcagacttgccttctccagtggggcaagtccccttggtggtgactgtcctggctgttgttccaatctgagctagtttcttcataagtcaattgaaggtgcagtattaccctgcagggtggaccaaggaatgtgttagccatgcttgctgcctttctccaggacctgaagccccgccaccctctcctggggtccctttttctctcccttctgcgggacacgttttgtgatctctgcgcaaacagaggcaacacacttagtcattaaggatttttctgtaagtgcattagccttaggcttcagcacttgctgttctctttgcttggaacttgttgaccctgctcttgtcactgggggctttgtcctcctgggaaactctgccttgaccacccatctagagtaaacgcccccattcactaccatggctccctactttctttgtggattttttttttttttttgagaagtctcactcttgttgcctaggctggagtgcaatggcatggtcccagctcactgcaacctccacctcctgggttcaggcgatcctcctgcctcaggctcccaaataactgagattacaggcgcccgccaccatgcccggctaatttttgtatttttggtagagacaaggtttcaccatgttggccaggctggtctcgaactcctgaactcaggtgatccaccctccttggcctcccaaagtgcttggattataggtgtgagccactgtgcctggccttctgtgtggatcttactgttactctgaccccctctttttagtgtgattgcctttccctctcccctctcccttccccaatgtgaactctgtaaaaatgatcctaggtctagaacctataagagtgcctgttagtggtgggtgcttaagagttaatttgaattaatgaggttaataattcttgtttccctatagtcctgttgttgagattcctggttagaaaaaaaagtgagttgtgggttgtatactttgtttatttatttattgtatttatttatttttgagactgagtctcgctctgtcgcccagtctggagtgcagtggtgcgacttcggctaactccaacttttgcctcccggattcaagcgattctcctgcctcagcctcctaagtagctgggactacaggcgtgtgccaccacactcggttaatgtttgtgcttttagtagagacagagtttcaccatgttggccaggctggtctcggactcctggcctcaggtgatctgcctgccttggcttcccaaagtgttgggattacaggcgtgagccactgcggctggcccgtgggttgtatccttttttattaataaaggcatctctatatgctgtggtttgagaggggttgaaggctacttcccatgggaatgagtgaggaagagcctattgcactctggtgtctttgagggaaagagaggcgggtggctgccttctctgctcttgggttccacagctggggattgggaggttactcttttatcaattggttttttctttttttctttttttttttttgaggcagggtctcactctgtcgcctaggctagtgtgcagtggtatgatcacagcttattgcagcttcgttcactttttttttttttttttttggagatgggtcctgtactgtctcccaggctcgagtgcagtggtgagatcatggctcactgcagccttgacttcctgggcttaagtgatcctcccacttcagcctctggaatagctgggaccacaggcgtgttccaccatgcctagctaataaaacaatttttttgtagggactaggtcttgccatgttgcccaggctggtctcaaactcctgggcttgagcaatcctcctgccttggcccctcaaagcagtgagattataggcatgaaccattgtgctcagccctgagatttgttttaaaaagtgccctgagattcactatggagtattccctcttgatttatttcctaccaagtaagccctttaacagaccatgtatggcagctttttgaagaatgttttaggtctttctaaatatgcagctattcaggctaaggattggcatgtttgtaattgacctaatgattcaaagaatggactggttcagctggaattttcctaaaatctatcccattgacagagtattgcacaattagaaaagtgtatgatttataattagcgtacccactttaactctgaataacaggcataaaggttattatattttgaatcagtgttaacagttttagacagctgttctttgttacttgatttttttttttttttgagacagaaacttactctgtcacccaggctggagtgcagtacagagatttctgcccactgcagcctgtattttgaggcacagttgtttttgctcattagagtgctgctgagagagttgatttcctagttctagagtcaccgccaagagtcctaaacattttcctataagagagatggtaaggatggttacaaagaagtaggcctgatttggtgtagaaaattggtttcatgttcaaagccaacaaggggatgggggagttttggatcagacctttgattctggcgcagagccacaagggctttgtgtgttttgtttacagagcttaagcaatctcagttattagcttgcggataattaccagaaatgtgaacttaaatctgtgcttatttatttaactccttgccttttctcctaagaatgtagggtggtggcattataaaaattaaagaaaaagcaaggctccaccactttcatggtgattctactttacctgcagtaaaatcacagcatgatggccaaattgcccttcctctccaaacttcataccatttactataaaataatttggatcatagggtgatgtaggcctggggctgaagtctgtgtcagaatgttcctgtggaggtttatatgttaaaaattaaaatgaaaacctgttttattttattttttgagacggagtcttgctctgttgcccaggctggagtgcagtggcacaatctcggctcactgcaacctctgcctcccgggttcaagtgattatcctgcttcagcctcctgagtagctgggattacaggcacccaccaccacacctggctaatttttgtgtttttagtagagacagggttgtaccatgttggccaggctggtctcgaactcctgacgtcaagtgatccgcctgcctcggcctcccaaagtgctgggattacaggcataagccaccatgcctggcttattctatgttgttttttgtgatggtcagtgagatgtggccacaggaggaggcagaggaggtgctcaggaaggccaaggttattatactcacaggtcttagagacgggaagcatgtcatgccctgcagggccacatggggaagcatcagtcttggtcccccctgtaggagcctgaaggggcaggagtgaggggagagcctgggccatggtctttcttgagctttcagggacagggcaggggagtgcatgtgaacagtttagaattggctagtttgaataattttggcaggctgtaagctacaggcatggtttctagttgcttgttatcaggctctggcatgatataggtgaagaaatactgcttcctggggtggacaggctagataaagacggcatagctctgggttggttggtttgcatatcagagctgtgctcctggctgggcccttctggttctaagaactggccagctctgggaaggacagtctctccccagccagagaggtttccaaaacgtcaagacatcatagtatgcacaaagtaaaaaatataaacagtacaaaggccctgaacagggtcaccctgctgtatgagaaatctactggcctggctgaaggggaaagttgcttgggcagaacagtgaaaagctaaagcaggcttgttagttgtatgagtctgttttcacgctgctgataaagacatacctgagactgggcaatttacaaaagaagaggtttagtggacttacagttccatgtggctggggagacctcacaatcatgacagaaggcacggaggagaaagtcacgtggatggcagcaggcaaagagagagcttgtgcaggcaaactcttatttttaaaagcatcagatatcgcgtgaaccccggggggcggagcctgcagtgagcctagatcgcgccactgcattccagcctgggcaacagcaagactccgtctcaaaaaaaaaaaaaaaaaaacaaaccaaaaaaaaaacccatcagatctcgtgagacttatccactatcacaagaacagcacaggaaagacccgcccccatgattcaattatctcccattggatccctcccacaacatgtgggaactgtgggagctacaagatgagatttgggtggggacacagagccatatcagtagtgtaaacctggagccagcaggaagaggcatttgacagagcaggagggtccatggtgctcagtcaaagtcttaggacaaatagtccccatggaacagaaggcaatgcacctggccttgttgcgtttaattttaactttgctgtattttcaatgatctgctcctccagggaactggtggcacaccctgagtctgctaggtgggctaaagactcacaacctgagggaaggggccaggaaagaaaggaagccatggcctagagggtgttatttgggaccccacgcccatccaggccgacagagctgcggtaaggctgagcaaaacacactcgcacctgaacctcactctgacgttgtgggaggccctaccttgtcaaagctgaaagaaacgtcaggaaaagaaaatgcccccaaataattgtaacacatgtgctattggtttggcgactttggtaaaaggtttggaagtttcagggaacattccagataagcagggaccatcttcttcataaattgctctttgaattaggataaaaaaaatcaattgccatagataaaatttctgggatttttatgagcccaacctgttttccaggttaattatgcctaaatatctgtttgttttctttcttgtcatataaagtcattggggtcctgtagggggtgtgtgtgtgtgtgtatgattttgtgttaggtagcaggtgtttaggtttgtccagcacatgtttattttatttttttatttttttattttttgagacggagtctcgctctgtcacccaggctggagtgcaatggtgacatcttggctcactgcaacctctgcctcccgggttcaagcgattcccctgtctcagcctctcgagtagcggggattacaggcatgcaccaccacacctggctaatttttggatttttagtagagacagggtttcaccatgttggccaggctggtctcaaactcctgacctcaggtgatccacctgccttggcctcccaaagtgctgggattacaggcgtgagccaccgtgcccggccccacatgtttagtactagatgctaaaatcaatctgtggccttgtggagtttataatccactgaattaatttagattaattcacaggattaatttagatttcatgtttgaatatgaaatttgtggtcatttaaacagaaactgggctggaaagtacttttttctctgtctgtgaaaggtttttttagggcaaatgaacagtataaattagattgcttagagaatgttgtaaacacattttaccacagacactttatgtgcacaaagaggcttggcgtgttggctcacacctgtaatcccacactttgggaggccaagtggatcacctgaggtcaggagtttgagaccagcctggccaacgtggtgaaacgctatctctagtaaaaatacaaaaaattagccgggtgtgctggcgggtgcctgtaatcccagctactcaggaggctgaggcaggagaatcacttgaacctaggaggtggaggttgcagtgagccgagatcgtgccattgcaccccagcctgggcaacaagagcgaaagttcgtctcaaaaaaaaaaaaatatgtgcttaaagaacatgctggagcatttcctctgaggaagttatctcaggtactagagattaatcagcctgcacctctttgtgtatattcaataatttagattttttgtgagttcaaactggctttttcttaaagttggtggggagaggcatgtgatcagacttcttgggtggttaggagctttttggattgagtcccgtctgttttccaggctgttggctaatggtgattaaaggcccagggaatcctgcttgtctccttccagtttcgtgcagagctatgtcacgctgcgtgctctgtttctgttaggaccaatgtctgtttgttatataaatagaaaaggaacataaagctaaggaaccaagaaggttccattacagatcagtgtctcaacgtatccttgaggtttggtacaagttctccgcttcctgaaatttttggacaaacattgtgacattcataaataccggactgctaggatattgagggctgaggtaacgcaaatacttgctgtggttttatagagtaataatctgttaatcatagctgtggccttagagaataaacaacctctgtgtaggcttctttgtaagaatggcacagctgacggggacctctgcgaaccaaaggccagcgtgtgaccttcttatcctctgcttcctaggccagcagtgactgcctttggagatctcattgtttacagccctgtctctgactcagggtggcagattatgtttttggtagagcccagccagcccctatcacattgcacactaatgtaatccgtcggcagctgtctctgtattcattatttagctgcactaactggagttaatcatagatctgagctaaactttgttatgggtcaagagagaaaggccatctggctatttcttgggtcgtcttgggtgtatttttccactgcttatcatttgaccaccatctgttttttgagacggagtctggctctgtcccccaggctggagtgcagtggcgcgatctcggctcactgcaagctccgcctcccaggttcacgccattctcctgcctcagcctcccgagtagctgggactacaggcggctgccaccatgccaggctaattttttgtatttttagtagagatggagtttcaccatgttaaccaggatagtctcgatctcctgaccttataatcgtctcgcctcggcctcccaaagtgctgggattacaggcgtgagccaccgtgcccggcccatttggccaccatctatgaggcatctgaaagaagagaaactcatgcgtggtggctctgcctgtagcttccctactcagtgtggtttgcagacagtagcattgccatcatctggggctggggagaaacacaggttctcatgctctgtcccagacttagtatcagaatctctactagatctcatgcgtgggccctggccagccccctgttgtgtgcttggttgacgctgggctgctgctggtagacctccctttctcttctgtgccacagggtctcaccaaactgcaaaggaggtgggtaataaagtcaaaccctgtgccaggaatgatgggagaaggaagggtcagacccacaaatgggccaaggaatggattaggtgagctaagctgtggttgtagttcagcagtcactattacgcccaccttttcttattcctttcctatttttcttttctttctttttttttttttttttcacagcctggtaatgtggaatatttacttttcttaaatcactgtattaaaagatgaaaaaagtcattttttctcttccccttcccacagtcattgttgattagagaaaatactaacaaaatatattaatttaatttccaactacagataacaccaatattaatagttgggtgactgtccctcttttcatggagaaaacctaatgcctatcaaatgcccaacgttgaagagctatacaaatgtggattttctggtcgtcttcctccctcccttcctttatcctctcttatgacagaaaattacaaacatgtacaaaagtagagggaacagtaatgaacccctgaataccagtctgtcaacaatgatcaattcccagccaatcttaaccctccttctactcatttcctgccctagaagtgttactttttttggcacatctgatgagtgaagtgaagaatggtatttcattgctctgtttttcttttctctattcttttcttttcttgcaaaaccccacactgtcaccatgcatttgttatgtaattttcagatgtatctgtaggaaatggtcagtatacgattatatatacactgttaaattatccattgaaatcacattgtgtaccttctcactacattttttaacctttatgttataaagcagatacagaatattatacaaacaaatgtgtaacgtggataataaattataaggttaacatcctggtaagtagcaaccagatcaagaaataagattttgctggtccctgttctgtgtcctgtctcaattccaggctcctcccttccctcagaaagaactgaaagtcaacttcaaactctttgtggtctgtattacttttaaatcttgatcaatgttgtgtgtatatatatatatatatatatatattttttttttgagatggagtctcgctgtgtcacccaggctggagtgcagtggcgcagtctcagctcactgcagcctccacctcctgtgttcaagcagtcctcctgcctcagccaccagactagcaggtgagctgcttgtgctgctgacaggttgtaggctctgtatttggaagatttaggtatggcaaatcagggtggcccagcccaccctgacttctagtcaaccagctacaaattctatgcttcccatgacccccttgggttcaataatctgctagaatgactcacagaactcaggaaagtgctacacttacaattacagttttactacagcaaatggctgccaattggaaccaaagggagagtcccaaatacaaagctttcttgtcccatctccagggagttgagacctgtcacttcctagcctattagtgaaggggtggccttcccctccacacctgtgggtgtttctcatcgggtgggacgagagactgagaaaagaaagagacacagagacaaagtatagagaaagaaaagtgggcccaggggaccgccgctcagaatacggagaacccgcgccaggacccgcgccccaggcaccggtctctgagttccctcagtatttattgatcattatttctcggagagggggatgtggcaggacaatagggtaatagtggggagagggtcagcaggaaaacatgtgaacaaatgtctctgtgtcataaacaaggttaagaaaaaggtgctgtgtgctttgatgtgcacatacataaacatctcaatgcattaaacagcagtactgccgccagcatgtctcacctctagccctaaggtggttttctcctatctcagtagatggaatatacaattgggttttacactgagacgttccattgccaagggccaagggacgagcaggagacagatgccttcctcttatctcaactgcaaagaggccttcctcttttactaatcctcctcagcacagaccctttagggtgttgggcaggggtacggtcaggtctttcccttcccacgaggccatattgtagactatcacatgggagaaaccttggacaatacctggctttcctaggcagaggtccctgcagccttcgcactgttttgtgtccctgggtacttgagattagggagtggtgatgacttttaagaagcacgctgtcttcaagcatttgtttagcaaagcacatcctgcatagccttaaattcattaaatcatgagtcaacacagcacatgtctctgcgagcacagggttgggggtagggttacagattaacagcatctcaaggcagaagaatttttcttagtgcagaacaaattggagtctcttaggtcttcttctttctacacagacacagtaacagtctgatctctctttcttctccccacatagtagtgtctgacaatgcccagagtattgccagccaggaaatctcatccaggttttggtgtctggagtttttattggagcttcattacatcggcacgattgacgggctcattggctgtgggattgatctcagtctccagtaccccctgccctggaggttgggcaatagcagttggctccaagcccagccctctcatcacaggattggtctttgtgatatgcacagcccccatcctgagtcatctgttagcatcagctgctagggacccactatgttatattatgtccctgataatttatgttagtgtaaaccatcagggaccacacttctattacttggagcattccaaggatttagaagaaccagggataaaacccagccaaattctttattatatgacttccttcccctccactccttactcttcatttctctggccctgtttactttgaccacccagccccttgtcaccatgcctgtagagctttgcagaagacaagtcaggggagctggtgtattagtccattttcacactgctgataaagacatacctgagactgggcagtgtacaaaagaaagaggtttaatggacagttccatgtggctggggaggcctcacaatcctggcagaagggaaagacacatctcacatggtggcagacaagagaacttgtgtagggaaactccactttataaaaccagcagatctcgtgagacctattcactatcatgggaatagcatgggaaagaactgcctccatgattcagttgcctcccaccgaggccctctcacaacacgggggaactgtgggagctacaattcaagatgagatttgatggccaggcgcaatggctcacacctgtagtcccagcactttgggaggccgaggtgggtggatcacttgaggtcaggagtttgagaccagcctttccaacataacatggtgaaaccccatctctactaaaaatacaaaacaaattagctgggcatggtggcaggtacctgcctgtaatcccagctacttgggaggctgaggcaggagaatcacttgaacctgggaggtggaggttgcagtgagctgagatggcaccattgcattccagcctgggcgaaactccgtctcaaaaaaaaaaaaaaaagatgagatttgagtggagacagagcgaaactccatctcaaaaaaaaaaaaaagagatttgagtagggacacaaccaaaccatatcagctggtaatgcagtgcacagaaacaggagagagcccaggggccaatgctacccaggcacctcaccaaagggataatgccccatttactaatgggggaagatggcctggctggctcccgagagcctgtcgcttgttggtgcgagggctctgaactctgctgtctggactagtctcagccctgtcacatgcaaactgcgtgccccaggacagctacatgtctgccatatgcctgagttttcccatccttagggaatggtgataacagcactgtatgagtttgctgaggctgccataacaaattcctacatacttggtggtttaagacaatagacatttattctcttaagttcaggaggccagaaggtaaaaaatccaggctgtgttcccttggtatggtggggacaatattccttgcctcttccagcttctggtggctgccggccgtccttagtgtccttggcttgtggctgcatcacttgagtctctacctgtgtttacatctccttctcttctcttctgtctgtcccaggtctcccccgcctctttcttggaaggacacttgccattggatttaggacccacttggataatccaggatgatgtcttcactccaacatcctcagtttaattccatgtgcaaatacccttttcccaaataacattcaattctttaccaggaaaggtaatactcacaggtactgggggtcaggacttgggcatatctttttggggccaccattaaacccccttcaggagcctacttcacaggattgtgaggattgcgttaaaatgcttgaaccagtgcctggcacacactaagtgctgttagtgctggcgactgggggggacagatacagcccgaccttttgtgtaatcttggccccttatttccccccattttagtctttcccccttcacagttatctaagtctgccttgggtaagcatatttaattttcatgctaagacatattttcaatctgtacggaagccatgcacttgcctctccttcagagctgggattttttttcattttgctggctgtgagcacacacacgcccacaggtgcctaagcctcttgtatgtgtgttttgaactgtgtcctctgagttctgtgtctgggtgcatgctctcctcttagcgtgggtctccttcccctgtgtagcacttcacaatgttaggcatttgtctgtgatagcagctgttcagtaatttcctacttgcccaaattcctggactcaagcagtcctcgagcctcagcctcccaaagtgttgggattacaggcatgagccactgtacctggctaatttcatgttttaccttgttaaccttttatttttatacttttaatctttttttttttcttattgtgtcatgcttttaaaccagtactctagagttttaatcattgtctttgaaatactttaacatctggtagtgtcttttaactttttattttgaaataattcaacctgacagaaaagttgcaagagtggtgcatacataccactcctgtatatccttcatccagattccctcctgttagcttctaccatagttgtcaatgtctctttctctgtttatattattcctttgttatctgaactacttgaaagtggattgtagtctggatgtggttgttcccacctgtaatcttagcactttgggaggctgaggtgagtggatcacctgaggtcaggagttcgagaccagcctggccaacatggcaaaaccccatctctgctaaaaatacaaaaattagtcaggtgtggtggtgcacacctgtagtcccagctactcaggaggctgaggcaggagaatcgcttgaacccaggaagcggaagttgcagtgagctgagatcacaccacttcactccagtttgagagaaagagcaaaactctgtctcaaaaaaaaaaaaaaaaaaaaaagtggattgtagatctgaggccccttatacctaaattttagtgcatgtttcttaaataaaaaggcactttccatcatagttatagaacacccaacaaaattcagaaatttatcttcaaactccataaccagtttccctatattaattttttgggcccaggatcctataatgtatttagcgctcatatttctttagtatccttcagcctcgaacacttctgcagtcttttattgtctctcatgtccttgatagtttgaaaaatattcaggtcatcacattctagaatattcttcaaagacacaaaaaaatgtgatgtgacttcatagcagaaataccataaaaataaatacagtatcctcagtgcattgtatccggaggcacacagtggtgatggtaactgttatcactctgttaaggtcttgtctgccacgtttccctgttctaaagttgattatgcaatactttatgcttttaggtaagtgttttatggggagataattggaggctatgaaaatatcttgcttctcatgaaacttttacccactgattttagcaaccgtggatggcttttcaataactgaatcacttattactgtggaagttgccaaatgatgatttttttttttctgattctattgttaattctgttcttaattggttggcattctacagtaagaaatattctgttttccccatttattcactcatttatttatagcactatggacttatgaataattttatttattagatttgaatccattacacttatgatttattttggtgctcagattgtcccaggtttggccagtgggagctcctgggtcctttggacatgttgtcgggattctttgaacatttcattacttttgtggcacaaaaagatagttcagacatgtcttgtacttcttctgccccgatcttggattcagccatttctcaaaagagcctctgcattaaaaagaaaagttacatattctttttcataagtggatactatatttagaaagagtgctgggtactatgcattttttttttttcttttttgagacagagtctctgttacccaggctgaagtacagtggcacgatctcggctcactgtaacctctgcttcctgggttcaagtgattctcctgcctcagtctcacaagtagctaggattacaggtgcatgccaccatgctgggctaatttttgtatttttagtagagacggggtttcaccatgttggccaggctggtcttgaactcctgacctcaggtgattcacccaccttggcctcccaaagtgttgggattacaggcgtgagccactgtgcccggcctaggcatatttattgttatgggatctcattgcttctaggaactctaagcggacaaagctagaatgtatttgcatacacagatcaacacatattatttatatatttgtgtgtatgtgtatatatatacacacacatatacatatacagttggttcttgttactagtggagctctataaagtctccacaaacacaggactagtgaatactgaaccattgcttccaggcgaaatacagggttaggttcctatgcgcctttggtcacatttttgtcagccatttaatatataaccttgttttatgcatgtgtctgcttagagacaccttttttaatatataattaatcattaaccttgaattaacagccagcagcactatagttcatgcctgaaggaagcttctgtaacacacgtttttttctgtagggcacattacacccgccttgcgctttgatgcactagactgcacttgagcatgatgcttggggccactttagacagcgaagtcacccacaaaagtccccaaaatgtgaagaacatggtactgaacagcccacaagaaggacagttgtttctactctaagagcgggaataagaaggcagagtgtcagctgggcgtggtagctcacacctgtaaatccagcactttgggaggccaaggtggctggattgcttgaggtcagcagttcgagaccagcctggccaacatggtgaaacctcgtctctactaaaaatacaaaaaaattagccgggcatggtggcgtgtgcctgtaatcccagctactcaggaggctgaagcaggagaatcgcttgaacccaggaagcggaggttgtggtgagccgagatcgcaccacagcactccagcctgggcaacagagtgagactccgtcttgggaaaaaaaaaaaaaaaagtctaagtgtcatttcgtttgacttcagctgggagcatgcatgttgggtgactcaaatattttgctactctgtgcatgtccgtgaaggactgcaagacacctggaggactgatttttggggttacaatttagcaagtaggcaaactcacaaatacaggatttgtcactaaagaggatcagtgtatattgatttctctatctttatttaggtatctatttatatagatctgtattaggaaacaaattagatagacttcacaccagtgcttcaaatcacagttcaaaaatctcatgctttgttctggttctccctctttccatatttgtatcttcatccttcagtagtgagaaacttgactcctgctgtcctcaacacgttgacttctttgctcagtcctcttgtttgtcaccagggccccagccacgtgggccatctccttgcagagagtgttcctgggacctggtgtgcggggccccagggctgtgctcccaggctgctgcttgtgaacttgctgagagctgccccccaggcatgttgccctaggctggcctcccaacctagtggatacttttgatcattgttcactgtaacctcaagcccctgggctcaagcattcctcctacctcagcctccagagtggctaggactacaggtgtgtgccactgtgcccagccaattaaatttttttttttttgtgtggagagaaggtctcactatgttgcccaggctggtctcgaacatttgggctcaagagatccccctgcctcagcctcccaaagcgctggtattataggcatgagccaccatgcctggcttgaagttaatttcttattaacgttttcagtatcattagaatatagtaatgcagtagatttttgactgtttattggtattgtacatcttgcacctctaatgtgctagcttactgtcttcagtcaatgtaggattggagttctaattgtgtaaaactctgtatataaggtttattacttttttttgtgtttttttcttcttcaatagccatgtcaccattaatactagtaaatggataagccattcctttctaatatcttgttttaatgacaatgaatttaagatatgttttgtggataagaagagctgctcatagatacagctttttatgttactgctatgctagttctaggtaatttcttttttttgatagggagtcttgctctgtcgcccagggtggagttgcagtggtgtgattttggctcactgcaacatctgcctcttgggttcaagtgattcttccgccttagcttcctgagtagctgggattacaggcacgcaccaccacgcccagctaatttttgtatttttagtagagacggggtttcgctattttggtcaaggctggtcttgaactcttgacctcaggtgatttgcccacctcggcctcccaaagtgctgggattacaggcgtgagccactgggcctgggaattctaggtaatttcagagtaaaaaatttgatttagagttgcttcttccttttctatgaaataggacatttacaaaattgtttttgaaaagatgacttgggttgcgatgtttttcttgttttgagatcctagagtaaatgagaaataggatgtggaattttaaactgacttcttgtgcatctgcttatttttgtggtagagtcacagagttacgaccaagcaggggaggtgagaattgtgcagactggggatattgggagggtgaagggatctagtttgcttgaataaaaggatgcaagtcactgtattggtgaagtttctttcaaatccaattcctgactgatttccttgccttccacagttcatcatatacaaagtagcttaaaaacattcacaatttccctgttatctttaggataaagtccaactttctgagtttagaacacaagacctttgattaccccattatcaccccagaccccatgcagcattttctctgctgtctccaagcaataagtcttggctgtgtggctccatttttctggaaaatttgtcccttcttcctcctttgtcttcttggtaaactccaaattcttttcctaaagtttgctcaaactgtgtccctttctccaaaccctctccaacttcccaggtacctgactataccctctttcttctctctaccgtacagatggttcccagacctggctgataaccaggatgtgctgggtgaggggagctttttaaggaccgccacttgggtttcatcttggcttgttctgattcagtcatttaggttgcggttctaggtgtctgtgggtttttgttttaaaattccccaggcataattgggtaacatattgaagtttaccgaaaaacaaaacaagaaactccccagtaaaactccccaggctgaggtagggattttgctaatgagccagggttgggttgggagtattggcgtatatgtcagacaagtgcttcccatttggtgtgtcaaagcaagctgtatggcacgttctcaaacctgaacctgcatccgcatcacctggagaacatcagaagatacagattgcagggccccactctcagagttcctgattcatagacctggagtgggtcctgagaatttgcatttctaactcttcaggggattctggtgctgctggtccagagatcacactttgagaaactctgctgtacagaagatgagtctcgtgagctgggatgttgatcccttagaaagcccagaagtagagtctgtgcatagcctgtgtccacatgtgtcccatcttttttttttttttttccccacactacacagatgtaacatgtcccatcttactgtaattctttgttaacatagatgccttgaactcttggagtggaattgaaccggtggatttactaatgggtttatatcatttgctaacgtggctaaaagactgctgtcattgtcttgcaacttctgtctgaggtcaacatttgatttaagattctatagcctgtccagaaaggctgtaaaaaactttagtgcggtggttctgatccatgtgattttgccaccctggcaatgcctcagataattttagttgtcaggacttgggatggggatgctgttggcttcttgagggtggggttcagggatgctgttaaccatcctacagtgcacgggacgacacccccaacaaagagtttttcagctccaaatgccagtagtgctgaggctgagaaaccttgccttaggggaaggcgtttctttttttcttccttttttttttttttgagacggagtttcactcttgttgcccaggctggagtacaatggcgtgatctcagctcaccgcaacctccacttccctggttcaagcgattctcctgcttcagcctcctgagtagctgggattacaggcatgtgccaccacgcctggctaaatttttagtagagataggatttctccatgttcgtcaggctggtctcaaactcctgacctcaggtgatccacctgcctcagcctcctaaagtgccgggattacaggcgtgagccaccatgcccagccgggggaaggcatttctaatgaggtagagtgggttttcctgtgagatctgtcattgtgtctccctgcacccagctcctgctctccagagtccctgactctgagcagcctacctccttctccaccctgccccaatgtagcccccaggccgggcccagcagcctcccgcaggcagggctggcagactcttcaaggaaaactaaccaatgattaataatttgccacacctgaaagtactttacaatgcactattctttcaataataaatgatttattgagtgaggctagagcatctgttcaatcatttactgacctcaagtgacctgcacattccccttctcctgcaggcgcttaaagagggttgctgggccccagatgtgggtgttctctgctgctgctcttcagcctgggtggagagtcactcactcgttcaccccgcagttttgagtggctcgcctggtccctggcccagtgaacaccataaaaaaatcctgccctcagagagcttttgttctagaggggaaagtgacaaagtaattaagtgcatttttttgtatctacacaaatagattgtagtaaatgggtaaagaaagtgagaatgaggatataaatgatcgacaatgagggtatgtttggttttttgtacacaagggtaggactttctggggattgagacctgcaggagagaagatctaatcatgggagaagatggggaaaagcatttgctcgcacgcaaggggacaaacgggaatgtgggagactgtcagaaacacatcaggccagggtggctggaacactgaacgaaggggacccagtcaagcacagcaccccaccatgagtttcattgtttttctactgcaggagagcagcatggcatgatatttttaagaatccactctggcttttctattgaaagatcacagggaaaccaagggtgaaggtgggtaggccagctagcaggctgttggggtcctgaaaattcatggtgccttgggccacagtgatggaggcaaaggtgtagagagaaagggttcttgttgggacaaatgggatctggttcttgttgggacaaatgcacctggaaaggcatgttggcgctcctgcaggcatgtgtgggctcacataagtgaggcttatgtgtggggcatggctgtgcgctgagttacgtgacacatgtcttaaattaaagacactcatatttatttcccagggacagataaactttgagcaatctgggaaagcatcaagataactgactaaattctatcagttgtttagccttgagaagtgtgaattacatgttcacatgcatgtaattcagcttccagagcaattctttgaccccagtgaagcacagttgacccttgaacaacaagggtttgaactgcaagagtccacttatgtgcagattttctttttttttctttttctttctttttttttttgagatgaagtctcactctgttgcccaggcaggagtgcaatggcatgatcttggctcactgcaacctctgcctcctgggttcaagcgattgtcctgtctcagcctcctgagtagctgggatgtgcaccgccatgtccggctaatttttctgtatttttatagagacagggtttcaccattgttggccaggctggtcttgaactcctgaccccaagtgatccacccgcctcggcctcccaaagtgctgggattacaggcgtgtgctgcgcccggcctacgtgcagattttcttcagcctcttccacctctgagacagcaaggccaatccctcatcttcctcttcctcttcagcctattcaatgtgaagatgatgaggatgaaaacctttgatgatccacttccatgtgtatcttctcctcaatgattttccacaatgattgtcttaatggcggtttcctttctctagcttactttattgtgagaatgtagtatataatacatagagcatacaaaatatgtgttcatcaactttatgttactggtaaggcttccagccaacagtaggctattactagttaaggttttggggagtcaaaaattaatatgtggttttttggttgcacagggggttggcaccccaatccctgcattgttcaaggatcaattgtatattattctctgtgcttcttggtgcgcaaacttaaatgagtagcttcagatgctcgctttgttaaggagtcttattccttagtctggcaagtgcttatttgagcttggaagcaagagagaactatgtcatctcttatgagagatgaagtttaatcgtttggaagaaagcgcttgatttgaacaggcactaattaatgtccagattttttttttgatttttcttttttctttttttttgagacagggtctcactctgtcacccaggctggagggcaatgagacgatcacgtctcactgcagccttgacttcccaggctcaggtgattctcccacctcagtctcctgagtagctgggactacaggtgcctgccaccacacctggccagttttttgtatgtttttgtcgagacagggttttgccatgttgtccaggctggtgtcaaactactgggctcaagcaatcttcctgcctcagactcccaaggtgttgggattacaggcgtgagccacagtccccggccaatggcctgattttcagaggatagtcaacatcatacactgatgggctcagaaggatcttggcaacaatgagccctgcatctagcaggtggccacacgggccttgctgactatatagtatggcatctctggctagtcgtcagatccttcacttgctagttgcaatgaacaatgctttcatctccttatgagtcagccgcataacttgggctgcagaaataattaatatcctactcacttagccgggcatgatggcatgcacctccagtctcagctatgcgagagggttgcttgagcccaggagtttcaatgagctatgattatgccgctgcaccccagcctgagtgacaaagtgagaacctgtctcttaaaaaaaaatcttactcacatttcttcattttattctgttactaaattgtgtacaaatatgccaagtgcatctttttttaaggaagaagactaagatggctaatcatgcacaatggctgtagatgcttggtactggttgtcgtgtgggaaagtgctgggaagatgggattataaagtgagggccctgtgaagaagtgaaaggcatggattagctggtgggagaacagggagacaagaaggctgtggatgaaccagagaaggctttgaagctccgtagtaggaaatctctgaggtgcctatggcatggaaggacccatgttgaacccagagtgcaggtgtggaggggggttgggggacccggaagagaggtggacagggtccacattgcctgttgcttaggttgacatagaaacaacaggtagtagagtgaagcgggaagggatgagaaaatgggctttgagcgtgttttaggaagggaggaacgttagaggaaaaataacagaagatcacagctttacaggtgccacctgagtttaatttacttggtcatgggccgagataagctgggttgggaccatgttgagaacacctgtggtgtgggtgctctaccctcacgcgagatccattccagcgatgcctgtgtctggtgatcgttgaactttagtgtctggtgatcgttgaactttagggcatggagaatggctattttggactacagagaatgggaggagattgaagaaatcaggtgtgtcgggctccccttggagccaggccatgggaaactttgtaaggacagaggccctggtgaactttggaaggattttgttttcttttaggcattcaggttttggggtctgagcagaattgctggagtgttacacgagaagtgtgctggcggtgggctacaccattccttcctccccagcactcagtcacttacctacgagaaaatgcacatttgcactggtgtgtgtccttgcaccctctttctgaaacccatttgcagaatccaggcctttaggaaacttttgctattgcagacttacttatatctgtaaattatttaatatttatatttgtaactgagaagttgatatcatggactcgtaatggattttggagtttaaatatgttaaaataggtaatttttacaggatgtgttcattcacgtgacaaatctgggtgacttctatgtgcccagcatgttgtaggtgtgggtggcttagcagcaaacaacacagaaaaaccccatccttgtggagcctagatgcctccaggggaaaacatgatttcaaaaaagtgaaatataatagcccttctaatgttgacaggtgctgagtagaaaatttaagcttgggcaggggcagagggtgagccttatagggtcaatgtgactgtttctggatatactttgcatgttgaagtaataggacttggatggcttagatgtgtcctatcaaagagtgacaaagaagggatgggctgcagagtcacctgaaggttttcttttctttggaaaatctgggcaaggggaaagatgtgacggctgataactgagagggagaagcctgggaggagcaggatttttttgtgggatgaaaaggtgttctgtttggacattatttagtttgatatgcttatcagacacccaagaggaggaggtgttgaatcagcagttggttatgagtcaggggtattgtatgtgcccttaggcactagcaatgttttttgtgtttttttttaagcaaaattatgaccatcatttcagaaaactattttatatatataaagaggttgaaagcttattaagactagatttagaaaaaacatataatactctgagaaattctgtgtgtagtgtcaggtagaaagtaaagtgttaccagttctgtttataaaaccccctttaggccaggcgcagtggctcatgcctataatcccaacactttgggaggccaaggtggggggattacttgaggccaggagtttgagaccagcctgggcaatataatgagactctatctttacaaaaatatacttaaaaaaatagccgggcatggtagcacgtgccttgttatcccagctacttgggaggttgaggtgagaggaccgcttgggcctgggaggttgaggctgtagtgaggtatgattgcgccactgcactccagcctgggtaagagagtgagaccctgtctcaaaaaaaagagaaaaatccactttatacaaaaatttgcatgagagtattgcccacatttgaaggaacacttaaccttccatgtattatgtttatatggagacttgtctctgaagatggattttggagtttatattaaatttgtttttgtttctgtgttgttttttttttgatggggtctcgctctgtcgcccaggctggagtgcagtgtgcagtctcggctcactgcaacctctgcctcctgggtttaagcgattctcctacctcagcctcccgagtagctgggattacgggtgtcctccaccacgcccggctaatttttgtatttttagtagaggcggggtttcatcatgctggccaggctggtctcgaactcctgacctcaagtaatccacctgtctcggcctcccaaagtgctgggattacaggcatgagccatcatgccaggccaaatttggggatttttggaggatgtgctcattcatgtgacaaatctaggtgccttctatgtgcccagcatgttgttggcttagcagcaaacaacacagaaaaacccagatgctctggggggaaaacatgatttttaaaaaaagtgaaatgcaatagcctttcaaatggcgacagatgctacagagaaaatttaagcgtaggtaaaggcagagggtgagccccatgggagggtcagtgtgcaaaatggaaactcagcatcatttcttcaatggagctgttttggagaaagcccggggcagtcatggttggccgtgcactaacctttctatcgtggaggcgtggggaggggagaggcattctcaggtgttttccttagaaacacaggaaggtgaggaagaacagtggaaactgaatgaggaataaaaggcattcagatttaaagataccaagattagaagttcagagtctagggtggtcaccagacagcagatactgtttaaaataagtgttgaattcctggcaccagaattctagtcctttactattttgtggcttccaaatagagaaagttcatggaaggaaacataaaggtcatctagttaaagtcctgtgtatctatttcactctctttatagatgtacagtagtccccccagtccgaggaggcaggggaactaccctcattgaagaccctcacctcagtggctgcctaaagccgtggatagtgctgaaccctactgattttttttcctacttacacatatctttgttggtttgtttgttttgtttttgagacagtctcgctctgtcacctaggctggagtgcaaaggtgcgatctcagctcactgcaacttctgcctcccagtttcaagcaattctcctgcctcagcctcccaaatagctgggatcacaggtgcctgccaccacgcctgggtaatttttgtattttagtagagatgggttttcaccatgctggccagtctggttttgaactgctgacctcaggcaatccacctgcctcggcctcccaaagtgctaggattacaggtgtgagccaccacacctggccctacttacacatatctatggtaaagtttaatattataaattgagcacagtaagagattaacaatgataataaaatagaataattacaacaatatactcttcaccatttcgttgatagaaggttcactcttaaaccagatctcagcaacctcagcatacaatttttttttccttttcttattggcttgagaaccttaaccttttcacataaaggaaggcctttgtggcttctccttggcatatccaaatggctagcatccttgtacttgctctttgggggccattattaagtaaaataagggtgacttgaacaccagtattgtgatgccatgacaatagatctgagcgcccagggtggctactaaatgactaaaggtgggtgacgtctgtagcatggaaatgctggacagagggaggattcccatcctgggtgggtttgtgggagatttcatcatgctactcagaatagtatgcaatttaagccataggaattatttctggaatttcccatttaatcttttcagtcaactgtgggtaactcaacctgtggaaagcgaaactgtggatatggggggatgactgtatagcatcgagagacacaagtgaactccttcagtagcctgccacaggggatttcattcaacctgcccttagccttccttgttctctaaactctcagcttagctcacagctttgctgtctgcaggtctgagcacaaacctgccttctcgcttacctcctggatcccgtgattccaggcctaagttcaggaggtatacagcagagaggaccttccctccctagtctcttcttttagtattgtttatttctaataaagttgggggaaacttttcttctttgatagactataattttagggttatgcaaatgcataatccattgaaaaaggttttattaggcttggcttctgaagaatataaaattttgaacttgtccattaaagagagaacaatcttgattcaatgagtatcaagtaatgaaataattattttagttccatcatggccagttgttacagaaaggtcaggaaagaggaggagaagagaaatagaaagggcctttaaggaggcagagagggctaaaagagagctgaaaggggacaaaaggagagagggaaagagaaagtgaggggatttaccaaagataagttagcttttatttcctcctttacttgtggctatggggaacatgaagctcatagaaactgaagactcaggtcagggtcacagagacagggagctgggacctgtgttttccctcttataccacattaagaaaccactattggccaagcttggtggctcatgcctgtaatcccagaattttgggaggccaaagtgggaggatcacttgagcccaggagttcgataccagcctgggcaacatagagagacctggtctctatcaaaaataaaaatattagccaggcacagtggcatgtgcctgtactcctagctacttgggaggtagaggtgggaggattgcttgaattcagaagtttgagactgctgagctctgatcggatctgagtgagctctgatcgctccactgtactccatccagcctgggcaacagagtgagactccatctcaaaaaaaaaaaaaaaaaaaaaaaaagaaaccagtattcagaacttctcaaatccttgggggaaaaaacaacaaaaactaaatgccaccaccaccaacaataaataacaaaactctctactaacccttgtctaagactcctgcttgctggaaatgatgctgggggttgtgatcctggggtggagaatgtctctgtattgctgaagctgatggcttcctgtgttgggcattgagcacgactatgctatattgagtttgtattgaattttaccctagatcattgctcaaggtactctacccatctgtcttattcaataatacaatcattcattctttccataactgtttattgagtatctatcacataccaggcataatggggattccaaataatatggcagtagtcattgccctgaaagcattgtactcctagtaggacagttaggagggaagcgatgctgctgtactaagtattgtgggagataagggggagaagagactaagtctgaggagagaagggctactggggcagaactgggtggaggaatggctcatttggcagtggtctggaattgttgactagaccccctcagggacacattggtattaaggagccaactgacttagatttccatgtataaagtccttacatagatggtcaaagataatttagtctcttgtcaaagttgacttttgtcatcaggcaaaagcctttagctcttgctggtacccacatcttttcagcccagctttttgagatctgggcaatgagagagagtaggagggtccttgttaagtccttactgatttgtgtcatcctctaatagaagctttgggtctcttctgattgcagatactttaactcaacagtggcatccctccttgttaatatttctaaatgttatgcagttaactgttcagcctgtggaacagagttaccattctcactttccttaattttttaacttcctggagccagttttattccctgattaaaaaaacctctttagtccaaaaagacaccagtgaattttacttagaagtccttgctgaaatatactgtatgaggttctgctggaagggaggtctcgggcatgcagaggtcatctcaggcatgtctgggaaacccacctaagttgaagctggactggttaggttacagtcttctggacacattttggaggaggtgaggagaaagaggtgagttgatgagttcttcttgccattgggttaattagaattgctagtttggatcagcaggaggtttccggaatcctgaacataacttggatacccctgcagtgtgtgctccaatcttacggggcattgaaagcccaccatttcactgcaggcagaagccaccaggcagtgggttccccatgggaagggcttcctagattagacaatccccatgttaaaataaaacatgcctattaaaggtgacacatggccagataagacagccaagatgaaagaagtttgtgtaattgagtaattgcatctatcaaagttattagaggtttccttatagggcacaaatatgtttcttcttcttttttttttttttagacaaagccttgctgttctgctcaggctggtgtgcagtggcgatcacagctcactgcagccttgacctcctgagctcagtccatcctcccacctcagcctctggaatagctgggatcacaggcttacaccagtacatctggctaaattttaattattatttatattttttattgttttgagatagtctcactctgttgcccagactggagtgcagtggcatcgatctcagctcactgcaacctctgcctcccaggctcaagtgattcttgtgccgcagcttcccgagtagctgggattacaggcatgtgtcatcacacctcgctaatttttatattttcttactagagatggggttttgccgggctggtctcgagcttctggcctcaagtgatccgcctacctcagccacctaaagtgctggggttacaggcatgagccactgcccagcctttttctttataagtttttagtagaagtgggcatctcactatgttgtgtaggctggtctcaaactcctgggctcaagccattctccctcctcagcctcccaaagctctggagttacaggtgtgagccactatgcctggtgcatgttaattattatcatttaatgattaatgatgttaattattatcatttaatgattaataatgttaatatcatttaagtgattcctaatggatgaccattttgatttttctttttttttagtaaaaagaatctcaaaatatttgaataagttctttctgtcctctgcaagattactattttgtcctttcgtaactaataaataatttgtacctagataattttggattatgtaaatgtcccatcaaaccttcactgactggttttagctcccattgaaaatgttttctaacctcatcattctttatctgagagtttttccttcttaccccatttatgtctcagtaaggattcgtggattcttttgttattcagtgggttagaattcattgctgtcatgatttatttggatgcttccattatcacagacttgtctagtgggaccccgtcaagctgggtcctctgttctttgggcccatcctcttgattccttgaatgtttccttatcgtatggtatctcatgggccatctttttctgccccagcctttgaatcagctatttcttcaggaagctctggttccttttagataataatggttttggaaaccaagatctgggcaccagcatgctctcagctactggtgtgtcattgcttctggggcctgtctgtggatagagctagggactgtgtgtgtgtgtgtgtgtgtgcacatgttaacttttctctctctagctctatatattcatatctgtctgtctatatatagctgtatgtattttaaaggtaagtttaagcaaaacccatggaggtaggagtctacttaaacacggatgtgtaattctggatatatgtctcttttctcttgttaacaggtggctcaatcccttgtttaaaattggccataaacggagattagaggaagatgatatgtattcagtgctgccagaagaccgctcacagcaccttggagaggagttgcaagggtaagtcggaggcagtaaagtgggcattaaggaagcgtgggtgtctgtgtgtggggagtgggttttgccttcctgggtcctgtctggagcctccgtttacccttaaggtgaatatgtgtcaccttcctcaggctgcccctaggctcagcctaccatggtggcctgggggaaactcctgttgcctgtgcctccgtagaggtcaggggagccagcaccaaaggcccgagatgcctgaggtctgtccctctaggtggaagtgttgtggttggaggaggttgtctggcttatctgtgctactgatggggcttcaatccagtaaagcttggcagtaacttgtttccccagacttcccctcaaggcctgtttttctgatgctccagattctgtaccacacataaccttcaggagccctgggagcaattagtagcatttatgaaatcgtcacaaaatgccctgtcttaaatgcatacctgtcactctgccatctcccctgttttatattgtggcagggttcactcttatttacgaagctttttcctcattgtaggttctgggataaagaagttttaagagctgagaatgacgcacagaagccttctttaacaagagcaatcataaagtgttactggaaatcttatttagttttgggaatttttacgttaattgaggtaaaagcttttacataccatgaattgtttttcaggacacatagagtaaatgtgctcgccattgactggtgctgtaatggatgggggaacagaagggagaagtggccagatggggtttgaaggtattttaaggtctaaggattaaacatgtctaggaagtattaggtagttagttcagtgagcgtatttatccaggtgaatacttaagtgactgaagttcaaggtcattattgtttttcttgtaaccaattaaaatgcagtgtcatggaagaagagtattttattctatggtaggacaatgttggtgctcggtaaatgttttaaatgctaagagtcactcctggtcaaaagaggatcctacagccttgaggagtgcgtgccctgtggtttagaggaagtgattttggatcggagcccagaataatgtaagacattttacaggtgctcaacaaataattgttcaccaagccaaatctgataattgctcttctggagaggtattgtcacctctcccctctcggggaaacagagctgagggccttgcacctgggctggcggtggtgccccagaggctggagctggctgaagcacccaagagctaccccctcccagtgggcccaggtgctgggagccaaaatcccctctgggaagaaggtccgaggactgtgcacccaaggggattcatcacacccatccggtgtgtcttccttgttgtcggagtggattgctggttggaaggtagagtcttggtgcctggccctgcagagaatgtataaatggacattccatgaaaaatcaaagacagccagaagggcaataacctctgttaccccgaagggcctctcttgagatctgcagaaagacagtgttctgcctaggcacactgccaggtagcttgtccgttctggtctttggcctgctagaggagatgcagagggtcctgctgggtgacttgtgtcctctgtgctaccttagggatgtcagtggtccattaaagttagaggggtggatgttgctgttgttacaaaagggtggctttaaatgcaggatggggagttgtcacggtgcattttgttgggaatgaggtgggtggggggagggggttggtttgtatgtgggggggtttgctcctgttgaacatgctttttgttgtggagggcacggtgggcagtgggaggcaggtaggtgagctctgccctacaccagaggaatctgtcagacaacaggggactggacggtcctgggagatgaatggtctcattggtgacaaaggtgttttggccagggtgccagatggtgaatgccacctttgagagggtacccgccctgcctgcatgctactgtggctttgtggcagatggggtggacatgcagaataaccggtggaactgacttgagggtggaagagggctaggagcccctcaggttctcccagcccctcttcaacctgtgctggggtgtcccagcgatagtgtttccacatggccccgatcagtttcagttggaaagaaatagaatgacaagtcggtgtcaatgtggccagtgtgtgcaggatttcctggcccagtttggctgtgcaacctcagagaagttgtctggtagccatggagcctctgtaagtgtctaaggctcatgccaggtgtctggttaggtgtagtgggaatcaatggcctcacataggtttcgaggcatgcctccctcagctacttccatttctctagtttccctttgtcaccagctgcttatgaaattgaaaacaggatgatttagaaggaactgagcttgtcatgctcagggctgtccatgctgtcatgggctgccagccacttggggtggggtctctaacccacagatggtgactggactacagtgagatttgggtaagcttttgggagaagcaggcacagaagtagaggctgtagaagcgtgaatttctcctgagacttgaggacaggccccactctggtgcaaggagcatggtaatctttactgctggtgggcatgtgggaaggggcccggatcgaggggagctttaatatgggtacagttggtggagagctggtcttttagcggggtggttattgggcagtcatggcagaaaagtagggagtggaggacctggcaaatatgggggctcatgctcagtactgcttagcagcagcagtgctagtagaagcagcagcaggattagtagtggttggtactcagtagtagtagtagcagcagcagtgatagtagcaatagttgtaataatactgacagtgagtacttgaaaaatactatgtaccaggaactgctatgatttctttctttctttctttctttctttctttctttctttctttctttctttctttctttctttctttctttctttcatgttattttattttattttgttttttgagatagggtctcactctgttgccaggctggagtgcagtggtgctatcttggctcactgcagctttggcctactgggctcaagcagtcctcccaagtagctaggaccacaggtgcacgccaccatgcctggttaatttttgtatttttgtttcactatgttgcccaggttggtcttgaactcctgggctcaaatgatcctcccaccttggcctctcaaagtgctgggattataggcaggaatcaccatgctcagtcagaaggcattcttttaaccctacttttgggaagaggacactgggtgccatgtagttatataatgtgcctagggccacacaaagtgtaaatgacagagcccagattcagtctcaggagcgctgacttagggacctaaccatctccagactgtcgtaaaggtgtcttgagaactctgtcttcagagtatccagcacatgcttcttgaacttggcctagggataagtcagatgctaagagctccctggaacagtggggcatggctgtgggaaatcagattggggattataaattaccctaggttcctaggtgtcctggtgccggaaagtcaggtgacatgctagagagtggctggggtgctgggcaggtcagagcaccaaggtacattcaggttgaccacgaactctttttttttttttatgttttcgtctgaggtggggtctcactctgtcactcaggctggagtgcagtggtgtgatctcagctcactgcaacctccatctccctgggctcaagcaatcctcccacctcagcctcctgagtagctgtgactataggcatgcaccatgcctggcttattttttgtatttttagtagagatgaggttttaccatgttgtccgggccggtctagaactcctgagctcaagccatcggcccacctcgccctcccaaattaatgggattacaggtgtgagccactgtgcctggctgaccaagaaccctttagaaacctgtctcccactgagtaccatcctccacatgtgtgaggcgtgcagccctgctgacccaagttcatcaacatccacgctgttaagacaccattcccagttgtctcctcctcggatggacgagggggaagcttgtggtcttgcatggtcacgacattgactgatggtctgggcggatgtcaagttccctgtttttacttatcaaaggctctgggtttcttgcatagcaatgatatctctgcagtttcagaagcctttggcctgaatcgtgaagtgggtgtgcagtttgcttggcaaggatgaagaccccagatgagtcactagtatggggacagctcttctgtcctggtatctgaaccctcccactagcaaggtgggccccaggtattagggcggcaggctaaggtgttaagatggacccacacagtgctggcggcaggcacctctcatataaagtggagcctgggcttttcacaagagagcacttgttggtcctgtgtttacaccaatatcttggtggtttatggtataaaattagacagtagtcagttattctttagaggatcatggtgactagtggatgatgctcattttaaccttgtgtctttaacataataactggtagaacttttagttccgatgcagtgaagttcagggagctaaagctcttcccagttcaactcaaagtaatccttggatttttttttttcttttccttctagcagtgaaaacagcctgaattctagtataaatactattgttttcccagccttgcctattgacacttggctgttttggagaagttggagagaaataatttcttaagatgaaggtagtgagtgtaacatctcaaaagcatttcaggtgttgttttgggttttcaatcattaaagctatttattggccaggcatggtggcttatgcctataatcccagcattttgggatgccaagggatgattatttgagcccaggagttcccagctttagtgagctatgattgcaccactgcactccagcatgggtgacagagcaagaccttgtctctaaaaaagaaaattaggccaggcgcagtggctcacgcctgtaatcccagcactttgggaggccaaggcgggcggatcacaaggtcaggagttcgagaccagcctgaccaacctggtgaaaccccatctctattaaaaatataaaaattagctgggcatggtggtgggcgcctgtaatcccagctactcaggaggctgaggcaggagaattgcttgaacctgggaggtggaggttgcagtgagctgagatcacgccactgcacttcagcctgggtgacagagcaagactctgtctcaaaaaaaaaaattatggagttagaacaaatgttagtttcacagttgatttaatgaagatgcttttcattttttggtcttgtgtctgtgctgcttgtggatcttcgtattttggatagtggaagctctaaacttaacctagcatttagtaaaaagcttctgtgatgactttcaggccttttttaacccacttatgccggaggttgccaattttttttgtgtgtgaaagatcagaccttggcgatgaggttgaacagtagaatataaactcccacaagcttagtgttccaataatggaatgctaggcacagatgggttaattcctctggttacagaacaactgtgcagtggatccataactccctagccatatactcttaactaagctgaaggcaagcagggccttccctgggtactaatgatggcaccagcaggaagagagatggtgacaccagtttgcggggagtctttgaggttggaatttcaaagtaatgcaagtgagattaggaacgattgtcagagttgaggaatgattataatgcccagagaaacaagtactagtctaatggtgaacatggtgctcttccaggcaaatgctaattagaacagaacagcccgcaaatttccagcacatctgtcccctatgtgcacctccatggagactgtgatggggactggagactgagcaagtatccacccacttagagcctgcagggggggcaggaggaaggaggagcttgggaatcctctgtgtgggccaaaccaggacacaggcagcttctccatttgtatctatgctgcttgttggtctttgtattttagatagttgaagattatctaaatgtttctttgagaaatgagatgtgtatttttttttttttcctgagatggagtctcattctgtcaccaggctggagtgcagtggcacgatctcggctcactgcaacctctgcctcccaggttcaagtgattctgctgcctctgcctcctgagtagctgggactacaggtgtgcgccaccacacccagctaatttttttgtatttttagtagagatggggtttcaccatgttggccaggatggtctcgatctcttgaccttgtgattcacccaccttggcctcccagagtgctgggattacaggcatgagccatcgtgcctggccaatgaggtggtttttaataacttcaattctacatttcttgttcagctgcctgacatgagggccaaggccaagcctcatgtggtattttctttctttggttgttttgttttttgagccagagtctcactctgtcacccaggttagagtgcagtggtgtgatcttggctcactgcaacctctgtctccttggttcaagccattcttctgcctcagcctcccgaatagctgggattgcaagcttataccaccacatgtagctaatttttgtatttttagtagagacagggttttaccatgttggccagcctggtctcgaactcctgacctcaagtgattcgtctgcctcggcctcccaaaatgctgggatctttcttaggtttttaaagatcatttcaaaaatataaaatataatagaatactaagcctctctctacccctaagccagcttcagtaactatcaactcatgaccactcttgatttttatgaagccctgctcactgccccctctgaagatgtgtgtgtgtgtgtgtgtgtgtgtgtgtgtgtgtgttttgagatggagttttgctcttgcccaggctggagtgcagtggcatgatcttggttcactgcaacctctgcctcctgggttcaagtgattctcctgcctcagcctcctgagtagctgggattataggcatctgccaccatgcccgggtaattttgtatttttagtagagatggggtttcaccatgttggccaggctggtctcgaactcctgacctcaagtggtccacctgcctcagcctctcaaagtgctgagattacatcgcgcccagccgcgtgtgtactttctaattgaagaaaccgtgttgtttgccctgcacggattcccagggtctagattgggctcattgcatatccctcctcccctcttgtgaccctctacatccttgcatgctgtatcatttaacatgtccatctggtaccagaagttagtagttagatctagaagcttgatgtgattcaggttttattttcccccctcctccctttggcaagagtactttgtaggtgagggtgtgtaccacctgccacttcgagatacctcgcttaggtctggttatgtgtgtgtggtatttggagccattcatgatcattgcttaagatgcattaatttgttaggagtttgcaaagtggtgatattccagttcctcatttattagctgaaatatttgcataaagagaaactttgccattatcaactctttgatactctgagttacagtacacctataggaaaggcaggagaaatgattaattttgttatttttttttaaaaccgattttcaaaataatgagtgattctccagcattttcctgtggagttaatggattttttgttattgcttgttctgtatattttgatgagtatgattgtgagctcatggatttaaacagttctgatgtgctgttcattacagttatcatccctgtgccacgctttggctgtctttggctagtgggagtctatcgaagttgggccctgagtcttttgaatgtatactattagtctttgcaacttcctcattttctggtatgacagatgttttaggctccctgaacagtttttgccccagctgtggaatcaaccatttttcctagcagctttaactccttttatgtttatatttaaagataaacatatcatgaatttatgtgggtatttccaaatcagattcaggacaccagtgttttgacttaatctctttactcttacatctgtatctcttttctcccttcatctgttcttttttctccctagcctaatcccagctcttgatatcaacatacttattcatttattttatcttacagtatcaacactattacccataatatgatttctaaaaataatttaaattttttttttttttgcagtttacattgtccttagggtagataataatgtgcagggactatcttttcaaactatttttttttacagtcaaataaactagttaaactattttgtttaaagttaaaaaaacaaattatgattcatccttttgtttggtaggtacagtctttactgaacatttaattttccctgctgtgcagaatggaagatcttagtttcatcactgaaatctcagcatccttttttcctaagggattttcttggaagcctaacatttggcagcagttctgattaaaagccctttgtggtcttaaaagttcttcagggtcaggcacagtggctcacgcctgtaatcccagcactttgggaggccaaggcaggcagatcacgaggtcaggagttcgagaccagcctggacaatatggtgaaatcctgtctctactaaaaaaatacaaaaattagttggtcgtggtggcatgtgcctgtagtcccagctactcaggaggccaaggcaggagaattgcttgaatctgggaggtggaggttgcggtgagctgagattgcaccactgcactccagcctgcgcaacaagagtgaaactccatctcaaaaaagctcttcacagcgggtctccagcccctccagactcagacacagagagtgcaggagaggagaatagtgatgtgtacaggcattggtttctacactccagaatcttccggggatggggaactcaggccctaccctgatcaattggccctaaatgtgggggatgggtcctgagcatcagtgagcgtgtaagtctcctgggtgattctaacatgcagtcaggactgcactgattcagagaggacttcaggtatatctggagtcttaatggttattatttccccagattcaccttttttcttccttatctctggatctttctcaaccctagttttgtcccctgttcccccatttctttttttttttttttttttttctgagatggagtcttgtgctgttctccaggctggagtgcagtggcgcgatcttggctcactgcagccttacctcctgggttcaagtgattcttgtgcctcagcctccccagtagctgggattacaggtgcgagccaccatacccagctaatttttgtgtttttagtagagatggggtttcaccatgttggccaggctggtcttgacctcctgacctcaagtgatctgcccacctcggcctcccaaagtgctgggattacaggtgggagccaccgcgcctggctgtatttccccatttctaaaacaaactgcttactcatttttccacagctcactcttttctgcttgtatttttcactcaaacatcttctttggtagcttgtttctatttcctgtgttttctttccttcccatgcaatctggtatggtgcagttgggcaaggaatatctgaagtgcgtgggggagcaggagcatggaggccgcgtgcccgctgggcctagacatgtaatgtgtcactccaagaaagcaagggatggggctcgaatgtcaggaggctgagtaccaaatactccattgagaagtatctgccttcaagtactctgggactccagtagaacctgaagtatggaacattcaagtatcctactggactctcaaaaacctccatggtttcacagtcgaggagaattcttgtgaaggagtaaggactattcttagcttgtttgtggaaagactcttcaccaggtcatagattggttggtctctaaggtggagaatttatgtgagttacttttgaccttggtactgatatttagaaagtttaaatttggttagttgtaattttgataatgagtattttcccagttgtttcgtttcttattagttgaaggttaagatggagaggtccgtggaaaagcatggaccaaaaaccagcattactcaccaatgagcccatttgcttgactggattattaaatgctgtctctcggcagggaaatctggacaattaggtagtcgtgacctacctgccttttgcattctctttcgctgggtttattgcatctgttatttcctctgggggtcatccctcttgccgtttgaatcatactcacctacggctgaaatggagaagcgtgtaacaggtaacagtgactcccagggactgtgagaagtttcaggaacatttagaaaggcaaagagcactgagcgtctggctgatagcccaggtgtgtctgctgcggccctggttgcccttggcactcctgagccagggctgcccatgtgtacagtttggccgtcttgcttcttgatgtaaacgtccagattttcatctcagtaaactctaaacagtccgtgttatgacccattgtactgggggctcttccataaactcttagtgccttttgaatgattggccatgctggaaatgtaatggtgagcctctgcattcttgatatctgatcatcttattcataattcttcaactaaatggtattttctgatttctgtatcataggggaaaacttccatgacctcatggaaggcagtgcagttggtgaagtgtgtgagagagagtgtgtgtgtgtgtgtgtgcgcgcacgcgcgcgttcacatgccgagacaggcttgttgggatttaatatcatgacccatctgagggatgggaatgtcttctgtaaatatcctgatcccgtgctagttgtttgggatgaaagtgtgtgttaattttgtttttgttttttactgattgttcttctctcacttcagatggacagaaaacagctatcaatggcaaacctgtcaggtgtttattatgtctttgttaagaaggagagattgcttaaaaggaaaagttgtttttcaaggttaaggtcttgaaagattttcaaacattttgcattttgtcttttgagaagtcaggaggaaatgatgtcgagggcctacatgtttacttttggtgacctggatttgcaatgagtgtttggttttctttgttgggggaaaagggtcccttacaacgggttcgagagggaggaagtaggttgccgggtgaagtgctcatttggcagggagggtaaaggaaagctcctttgcttacctcaaaccgtggctcaagctgcgttctgctgtttctttgcctttagtcttggaggttaaaattgcattccctacatcttgttcagaaggtaagcccgcatgcttcccagctcattggtgtgggattttaattttgccttggggaaaagatgattattaatattgcttcaaaaccaggaccttcaaaatatggctgataaagagagccactgatgacattccctttgagctcaatttctgctgtgtttaatttttgactccacactacctgagccaaagtggcagttcttctgggcacatccgggcttgtagggggcagaaacatactgaagcaatgtcctgtgggtccaagagcctggaatcctcccaagtctggttcttttccccactccaactgaagctggacttggtggtgttagatctaccgactcttggagtttggattgggaaaaattggagagtttgattttctgatctgggtttgtgccaatatctccattcacaaaggctcttaactgtgaagaaggtggcaagagtgctggcctcccatagacgagcctgtgggctgggatgtcaagggtacagaggaagtggggtgagagctggggtgtctgtttgtaagcaaattccttactcctcttgggccatttttttccttattcaccttaggccaagacttaatttggtagtttgccaagtgtgctttaaagccctgtttgtaatacagagcacccacagtgtaccaagcacaatgtactaaaactttattgactttgaaattagaattttgatttcaggttaaaactcctatcggcttgtcttaaagaagaaaacagttctgcttttcttttagatgagtgatgaatgttgcactttgaaaatataaaacttgtgttaagaaaaaacgagcctaggtgccgtggctcacatctgtaatcctagcactttgggaggctgaggtgggaggatcacttgaggctaggagttcgggaccagcctgggcaacatagtgagaccctgtcttgaccaaaaaaaaaaaatttagctctacatgatggtgtgcacttgtagtcccagctacttgggaggctgaggtgtgaggattgcttgagttagggagttcgaggctgctgtgagctgggattgtgccactatactccagcatgggtgacagagcaagcaatatctgaaaaaagagaaagtaaaaagaaaaaatggtgtggtctgtatgtctaattaaaggcttttctttttcttcttttaggaaagtgccaaagtaatccagcccatatttttgggaaaaattattaattattttgaaaattatgatcccatggattctgtggctttgaacacagcgtacgcctatgccacggtgctgactttttgcacgctcattttggctatactgcatcacttatatttttatcacgttcagtgtgctgggatgaggttacgagtagccatgtgccatatgatttatcggaaggtaagtgacattcagcattaaacatgtacctcacctgaagcatcagaagcattctgaggaagttttatgtaaattaaatactgcataaatagccactgcactccagcctgggtgacagagcaagaccctgtctctaaaaaaagaaaaacgaagaaaacttctgtaaatagaatcatttacaccttactagagaagcttgctatttgcatcaggccaatgttttttttctttatgcaaaattttccagtgtataatgaaagttttctactgctgtcagggcatggctgcctttgataacaggctgttgcagaatactaaatttataatgaaatctaattaaaaatatcaaattggttttcagtgtttctttgcctcaatctcatagttgtatttatggaaatttttgttgttgttcattaaaaccttcacttacatcacagttgtattcagaagtgaacttcagtgaaattggatctatgaaattaaatctaggagaaaagaatatatttgagaaagggaattcttatattttttaaatgaaccatttagtcatattccatataattttcttaactgaagaaaaatgtaaatggaatccacgtcggatacaggctttctttatattctaaatctagttgtggttgtttttaatggtatttaagaaaatagttggatgtttgggataacactttgaattgttgattgtttttcacaattttggagagaacttatagtgtttctattttatgttggttttcattcaaagtaagtttttacattaaacagttgtcccttggtattcgtgggggattggttacaggacctcttgaggatacaaaaaaattggaggatgcttaagtccttgatataaagtggcatagtatttgtatataacttattcacaccctcccatatatggcattttatttatttatttattttaaataatttcatcttttattttagattcaggggtagaggtgcaagttttttccatgggtgtattgtgtgatgctgaggtttgggttatggatgatcccatcccccaggccatgagtgtggtacctgataggtagtttttcagcccttgccgcattccctctctcccaactctagtagtcttcagtgtctttgttctcatctttatgtccatctgtacccaatgattacctcccacttataagtgagaacatctggtatttggttttctgttcctgcgttaattcgcttaggataatggtctccagctgcatccacgtggctgcaaaggacatgattttttttttttttatgggtgcactcccatgtactttaaatcatcttggtttacttataatacctaatacaatgtaaatatacattatataatgtacaatatacaatgtacattgtgtacagttatataatgtacattgtaaagtgtacattatttaattgcacattgtaaattgtacattatataattgtacattgtataatgtaaatatacattgtattaggtactataaacaaataactgtttagggaatagtgacaagaaaaaaatctgtgtgtgttcagtacagacacaatcacctattttgtttcccccaaatattttcaaccccaggttggttgagtccagggatgccaaacccatgcacaggaaaggctgactgtattttctttcccattagggcagtaataatgataagacctgatttgatattttcttattttatttgtgcttttgtattgagatatgccataaataataccatagtttgctgtggtcttaactgttatagaaggagtgggatttctggttgctttgctgagaagttgccatagagatgctagtgtcaagagctaaagaacttgtcattaaatcagtcctggatatctctttgtcctgggccgtttttttaagccatttaaatttttttttttttttgagatgaagtcttgctctgtcgcccaggctggagtgtagtggtgcaacctctgctcactgcaacctccacctcccgggttcaagcgattcttctgcctcagcctgccgagtagctgggattatggacatgggccaccacacccagctaatttttgtatttttagtagagacgtggtgtcaccatgttggccaggctggtcttgaactcctgaccttgtgatctgcccgtctcggcctcccaaagtgctgggattacaggtgggagccaccgcacccagcccatttaaatttaaagtattcttctttgacctcgttgtcacttatccagagtgtccgtaaggtgtcctccccaagtcttcctttgacatgggggaatcaatcttataattctgtgaatcctggaggcctcgtgtcttagtgtctatggtacagtataacaggtatgcccggattcctcatgtgtgacatgaaaatgataacaaggcttgtacacactagtcaaggggtcttcatcatcatcaccaccaccaccaccaccaccaccaccatcatcatcagcagcagcagcatcatcatcatctgtgttattctcctgtactctcttctaccatttaggagaagaatactcaggtttttctataaactagctagatttttgtctttcaagatcatacttctaataaattcataaaaattttcactaacactccacttggcaaactttaaaaacaccctacatgaagtccggtttgaggtagtggttcagtaaaacacagctctttgtacaagtgagatactgttcacctgtttttcttcctcttttccactaacgctttggaaaatagcctttgggaaatctgatctaactcggcagacttgcttgcctgcatgtaaacatctcccaaaggtattaatttgtcagcttgattatcttagaagtattgtctctttatgaagtcaaaaattgcttgggttaaaccataagacttaacctgaaatcccagaaacgtgggaaagacgaatatcctcatgtcctgccttgtatgtggaaacatgcccaggtgaagaaataagacacttcctcctcacacgtgggctgcttccttggggcagaggctggctctgcagcaggactctgaaaggtccctctgcgacactggcagtttccactgaggcccccatatcctgattcccggtctggcagaaatcaggttggctgttgcaaatggaatgactgccgagcccgtcgtcgacatttggtattgaatgatcttgcaggatgtctgaaaatgactcgtcatttatttactttttcacagctttggtaaaattactttaggcttttcatcattaatttttcttcttattgtaataaaatatgcataacataaaatttaccattttaactctttttaagtgtacagtagtagttcgtttgcattaagtacattcacatcattgtgcctcccaaccccaccatccatcgacagaatttgttcatcttcccaggtggaaactctgtacccattaggcactggtgtccactctcccctctccacagttgtcagccttctgctttccagctctatgaatttgatgactctaggtatttcatagaagtagaatcatacagtatttgttattttctgtctggctcacttcatgtagcataaggtcttcagggattatctgtgttgtattgtatgccagattttaattcctttgaaagagtgaataatattccactatttggatatgcctcattttgtttatccattcatctgccaccggacatttgggttatttctgccttttagctattgtgaacaatgctgctatgaacacgaatgtacaattatctctttgagtccttgcatttagttttcatttatttttatttttgagatggagtcttgctctgtcacccagtcaggagtgcagtggcatgatctcagctcactacaacctctgcctcctgggttcaagtgattctcctgcctcagcctcctgagtagctgggactacaggcacctgccaccacgccaggctaatttttgtatttttaatagagacagggtttcaccatgttggccagactgatctcaaactactgacctcgcgatctgctcaccttggcctcccaaagtgctgggattacaggcgtgagccactgtacccggccgagtccttgcttttaatacttttgagtatatatttatttagagatggagttgctgggttatgtggtaattctgtttaattttttgaggaaccatcatactgtttttcacagtggccacaccattttacattcccacttattctttaataatttgtctgtttaacttgagaactaaatgagatgtttttatgaatcatgactagctttaatttggattgatggctccaagggtggtctgttgaaatggttcatggtggtttgttcaggttcctcggaactctgtgttatttcttacaacttttaccaaataaaaagactgtgcagcacaggctgtattttaagtgaggtcaacagattggcattaagaatatgggctcctcatggcaatagtgtgctgaacattgtacttgctcatgttagaatgtcgtcagtcacaagtcatattcccagcatgggttcgtataacctctgcagtggggacagtgccttatctcctgtttttactttttagctatctgggacacagaagcaggggatgtgtccatcttcaagtctcaaggggttacttctatagcttagagcacatgagttggggagttggctccatgttggaggaagagggtccctttagtcaaactgagcctggattaaggatagctgaagatcttcagccccagccaccagtgagactggatctcccatttgcccttgcctaccttgacttagggccaggatagatctgagcctgaatgctaaacatctgaatgctagacctacgatggaatggaggacagtttcattaatcaggatgggcttgtcatcccagcttttgtattatcaaatacattaggcatagagacttttccttaagatctgtggcctaattagctcaagatttcttgactttgtatctaatttgaagaaccgtggtggttcctgcagtgggttggcttctgagttgtggattttgatgttgggttggcttgtcctgaatgccaggtaaagctacagcagtacctgagcctgcttgctggtgctcctctctgctgtttgcagacttttttgcagaattgagaaagtttggggaaactttttattctggataaaactgctcgtcttcactttctttagagttactctgaaggatagtttgaggtaaagtatctgtctgtccttcatcttccaccttctaaggtgtgttgagtccctgcagactttctagttttcctttatcatttattacagatctgccctagaagaatatatggaatatatgtatgtagtgtatctcagaggggtgctctctatagatttctgtgttgagaaatatttgtagttaccctaagtgtattgtttaaccatttatgtgatactgtgtggttctctttttagaattctgtaacttggggggaaatgcatattccctgtatgcagggaatatggatactccaggaatacatgtatatattctcttggatgctccatccagagttcatattatcaggtactcttcatctttctgggggaagaattcctgtttcttttaatcttttcatttgagaaccctctcctcttgaccatttcctttttttttctttaaagagacagggtctccctatgttgcccaggctggtctcgaactcctgggctcaagggatcctcctgccttggcctcccaaagtaccaggattacaggtgtgagccaccactcctggcttttttttttttttcaagacagagtcttgctctgtcgcccaggctagagcacagtggcatgatcttagttcactgcaacctccgcctcccgggttcaagtgattcccatgcctcagcttcccaagtagctgggattacaggcatggtggtgcatttttgtatttttagtagagacggcatttcaccatgttggccaggctggtcgcgaactccaaacctcaggtaatgcgcctacattgacctctcaaagtgctgggattacaggtgtgagccactgcgcctggccttgatcttttccttctcttttcttggcattgctgcagggcaggtatatttttaggtcagtggtcagagcagcagacagcctcctctaccacagactgagtaggaagtggaaggtgccctcccataatgcacagagaggctgggcttgtgcacccttcccaagtcatgtctgccgccctatgcaataagtagaacctgaagtaaaaaaaataaatgcaccgtcatgcgctgatggtgagaagattgcagccacctgtagctctctggctgcatcccagtcttcccaccccgtctccctctgggttttctcagtggatgtggacagacaattgatagcaaattttgtcagcaactgagccatacagtgttgtggtcttagttttaaaacagaacttttgtttttatttaaagctatttttatttcactgtagactgtatattgctttatatttagactgaatagtatgaaattacagatattcagggccgggtgcagtggctcacacctgtaatcccaacactatgggaggccaaggtgggtggatcacttgaggccaggagttcgagaccagcctgaccaacatggtgaaaccccatctctactaaaaatacaaaaattagcctggcatggtggtgcacgcctgtgatcccatctacttgggaggctgaggcacaagaattgcttgaacccaggaggtggaggttgcagtgagctgagatccgtgccaccgcactccagcgtgggtgacagaacaagactcttctttaaaaaaaagaaattacatatattcaactcctttgggcctttttacaaaactagcaatttcatatggctcaaactaatcttttgtctattagtctgtagatcggtttcctttctgaagattcgtagagagcctcttgacagtgttgtcaaagtgaaactgtgtggtgtgtgtggaagttatctccctggacacatgtgatactttggttggacttcctcttctaatgttattttctaagtttctgacaaaattagaatgacccagtagtgcttcttacattgtccataatacgttaaaatttttgtttcaaaaacttttctcccccacagtttttggttttacttctgatgtgaacttgaattgtacctgggcattttgtgagacattaatctcatcaagggacaatttgtcagtcttctagtttgctagggaatagcttaagaggagccaagttaactttccatagcttggagtttgagctaagaaagcaagttaaggagggaggacttgttttgtttggatttttccaagaggaaatttctgctgctggagatttaggagtgtcagatgcgtcccatcatctgataattgtatttgcttcactggggtgtgggtgcaaactggaagaaaatgctttagctcagtggttcctcaaactgctctctgttgtattcatgaggggaaatctttaaaataccactggtgtctggactcttcccctaaacattctgatttaattgctttagggaaattttaaaagctcctatgtgatactaataggcctcaaatatgtgttcagcttaattttatgctttataactaagaccttccagttcacagagatagcatatgggttgcgggtttaatccccctcattggtgaataactccttcggacttgtaggtataaacctcaagcatggtagctgttcatcagcccgagtgtcctaccatctggtctgggaatctcatatgcaggagagcccgggtgttgagtcaggtgtccaggcactttgtagaagactccatgaagtcctctagtggcagattggcaagggtagagattgagtgtggactctgagagcaatcatggactcagagcagtcagacctgagttcacatctcagtgctgtcattttctaagctgtaaaatcttaagagaagcacattcgcttcccagggccagtttcctgataacaacttgttgaagactactctttccccattgaattaagttggagaaaatgtagattagatggccatttatgtgtgaatctttttctggatcctctattctgttttgttgatctatattttctttgttttactttagtagtcagatggatagaatctattctttcttcatagcaaattatcttaattactatagctttataaatcaaatagtgaaagttacccaacctgtttttcttttatgattgttttgactattctaagtctcttgcattttcatattgactttagaatcagcttttcaagttttacaggaaactgttgggttatgcatcttgaattctaagacatggtcttagaattccagatgaagtaattccagatgaagtaattgccctgttggagaagtaattctccaacagggcaattctatccacctgactattaatgttaagagtgggcatcttaacattatttagatttccagtctaagaaaatagtgtatttacctattcctcagttatctacatctttatacatttctttttcttttttttttttttttaagtggagatggagtctcagtatgttgcccaggatgatcttcctgagctcaattgattctcctgtttcagcctgccaaagtgttgggattacaggcatgagccactcacgccaagccctagatctttaactctccttgtaattattaggatagaggtcttaaagatcatttgttatatttttatattttatatttttatatttttatatttgttagcatttaaaaaagaatgccataattcaaagtagcatttgtgctgttataaatggtattttatttatttattttatttattttttgagacagactctcgctctgttgcccaagctggagtgcagtggcacgatcttggctcactgcaacctccgcctcctgggctcaagcaattaccctgcctcagcctcctgagtagctgggattacagatgcatgccactgcacccgactaatttttgtatttttagtagagacgggattttaccatgttggccaggcaggtctcgaactcctgacctcaggtgatccacccaccttggcctcccaaagtgctgggattacaggagtgagccactgtgcccggccataaatcgtattttaaattttcacttaccgtttgttgttagtatatagaaatacaattgatttttgtatttttgctttgtttcctgaaaccttgctaaagtgcgttaagttgcagtaatttttggctgattccttaggatagctatttagataatcatgcctttggataaaaacaaatatactctgtgtgtgtgtgtgtgtgtgtgtgtgtgtgtgtgtgtgtgtgtgtgtgtgagagagagagagacagacagacagacagacagtgggggagagggagagaagaagggagagaagttaaaatctccggttatgagagttagtctctttttctccttaatcatgaactttttgcatcctgacttactggattcacttgtatttaaagtttcttactggattcatttgtgcttaaggtttatttgtatttaatatcccctcatcttcaccgccatacaatatttatgctttaattattcagttgtctttccacaatttaaaagaagaaaaaaatagtttttaaattacccactgaattactatttctggtgctcttcattccttccctttagctccaggtttccatttgatataattttttttcagcatgtaaaacatctattagcatttctttattttctttttttttttgagacagagtctcactctgtcacccagactggagtgcagtggcacaatctcggctcactgcaacctccgcctcccgggttcaagtgattctcctgtctcagcctcccgagtagctgggattataggcatgtgacaccacacctggctaaattttgtatttttggtagagatggggttttgctatgttacccaggctggtcttgaactcctgacctcaagtgatctgcctgccttggcctcccaaagtgctgggattacaggcgtgagccaccacgactggccaacatttcttatagtagaagtctgctggccatgaattctctcattttgttctttaatctgaacaaatttttttttttcttttgaagcacatggttgctggatgttgaattctgggtgatagctggtggtgtctgcccctctcccttcaaaggtgttccattgtcaactggccttcattctttctaataagttgttagccatcatttgtatctttattaccttgtttgtaatgtttctctttttttttttttgggttgatttttttttctctttgatactgattttttagaggattgataatgatcctaggtataatttacttggtatttatacttctcggagtttgctgagcttcttggatctttgatttgattctttttattattgtaaagtttaggaaatatttaaccattatatcttaagttttttttgacttattctcacactcttgtttttcttggactcctattacatatatattaaactatttgagattgtcttataggacagtcagtctttttcacttttattcaatcttctctgttgcttttaaactgctgataagaccagccagtgatttttcttttctcctttatttctcatttcagtattgcagctctagagtgtttattgggctctttttaaaaatactttacactggccaggcacggtggcatgtaatcccaacactttgggaggccaaggcgggcagatcacatgaagtcaggagtttgagactagcctggccaacatggtgaaaccctatctctactaaaaatacaaaatttagctgggtgtggtggcggacgcctgtaatcccagttactcaggaggctgaggcaggagaatcgcttgaacccgggaggcagaggttgcagtgagctgagatcacaccactgtactccagcctgggtgagagtgagactctttctcaaaaaacaaacaaacaaacaaaaaactttacacttttttgctgaaaatctctatctctttactcatatttttcctcagtttctttgaacatactaacccattgtacatttatacttgtttctgtaaatcttaaaatccaatgtgtgtgcctacttggatttacttttattatttatatatttgaggggtttggtttttattatgaagactgctttctttattgccctgagttaaattttctttcttgcatgtctaaggttttttattgtggacattttggatgatacattgtatgtctgacttctgataattctaaaaagtattgaattttactccaataccatattaactggctgaactcataggcctgttttttatgctttgttagggcagaactttggaaattttcaggtgttttccaagcttctctaacttgatgggactcaagattcaaaacatgtgtcatttgcaggccctgttaggtagtgcttgttttttcttgggcaagtttagagttaactcttttttagtgctcaacctgggtagcacttgacatttagggctaaataatttctttttggggatggagaggctgctccgtgcattgtaagatgttgaacagcatctgtggcctctgcccactagataccgaaagcgtccctctagttgtaacaaccagtagtgcctccagtcgttgccacatctctcctgggaggcaaaattgccctgtttgagaattatttctctggatagtgctcttgaattctaagacatgatctctctggtgtttcaaccgtatgccaggtgtgttaatgatatatgaatgaggtttccgcactctggtctggtgagaatttcagtacctcacctcagcttgaccttcagtatctctattcagctcactcctcctcagcaccagctctctgttaaggcttgggcggccttgacctgtgcccgcgcatcagccaaggacttgtagcacccccaccccacagccttctggggcccttcccttctgatacctgcttactgtccagggccatgcagatgccagtggctttaactaacccaagctctggcctccacccctttgctcagcagggctgctgtgatgtgtttggacttcagctcactgccacagtcggaaagtggtgtcctagctcagagccaaggcaatgcttggtgtctggagacatctgccttctagtgttgtacagttctgaggcagaggagtgggccgttatggtatgagccacttagccgtggccagagcccagtctctctgctagttgaatagcccagatgtttatgagctgtggagctgggagctccaagtgtacagtatagggtgtggttgtgtgagctgtaggacatgcctgtttaacttccttactgaatagatggagggtgagggcatatgcaagactgaggggctccagcatctgccatatgtcacctgtgtgcttaacaaaaaccccaatcctggggccatctcagacctgacctgctgtttcagaatttctagagaaagggcctggaaatctgtatatttaatttgtgcctcagggaagtttctgtactagaggatggcgaagggaacaacaggtgtggagagggaggctggacgtggcttccttgaaagggaaagggccgcatgaggctctgcattcctttccccaggacttatattgaaatcatggccaatgatttatttattttttaaaaatattaattcagtttttaaattttagttttttttctgttttttccttttttctatcttctaagacatcctgttatagcaaatttttacattttttcattgccctgccctgaaaatgtttttatttctcgtttttttttttaacatatttctctaggtttagaatttgtattttgtgcttatttactgtcaggccttcaaacataattgatttgtttttaatctttttctactaaaaattaaaaaaaattgtggtaaaatatacataacggtcaggcatggtggcttacacctgtaatcccaatactttgggaggctcagatggctggatcgcttgagccttggagttcaagaccagcctgggcaacatagtgaaaccctgtttctacaaaaaatacaaaaatcagccaggcatgatggtgtgtaccagtagtctcagctacttgggaggctgaggtgggaggattacctgagctcaggaagttgaggcttcagtgagccatgattgtgccactgtacgccatcctgggcactggagtgagaccctgtctcaaaaaaatatctgtcacataaaatttgtaatttttgcaaggcatggtggctcttgcctgtcatcctctggtgtcctccattttactttctgtctgtaataatctgattactgaagggacctcatccaagtgggatcataccatatttatccttttgtgagtggcttatctcacttcgcatcatgtcctcaggtaaatttaattttggggtttatgtcatacattttctcagcatttagtgaaaaacttctgcctcaccctgatcctcaactgtggtgctgaacctttatttagctgtagtggaagaggaaagaatttctgagattctacccttttctggcttcttgctctgagcaactgcctgtgtcctgatgtgttttgaagtcctcatcttctcctttctactccctctgggacccattagctgaaggaacagggtcattttctcttttggatctctcctgggggtaatagctgtgggcacagggctgttatttgatatgcttatcatagagtcgagaaagtgggaaggaggtaccttggtaaagagtgtctgtccctcctctctctgtgcatttttctcctaattcctttatcctcatcagtgaaagagatgaaggcattactgcatttggtatgtttggttgtaaaaatgtatgttagtgaataagtcattagtaagacattttctgacatctgtcattttttttcctgctgcacaaatctcttcattttcttaaagcttcaaagataatcttgaaatgggaatgaatgatggaatgaattttgcagtattacagtactgtgtactgttactttcggattactgaatatctggcctactcaaggtcaatactttgataaatacacaacattaggaaaaaaggaaaatcacgtatgtaaaatattttcattttgactgattcttagtaaaattttggtgagtctgtagagtgaagattgcttcctccaagtattcttagtcttaaagacggcacatgaaacatgtcatatttgagaagatacattataaacacatttcttatttgtttcttataatattcttttagaagcagaaagcaggctgggcacgttggttcacacctataatcccagtactttgcaaggccaaggcaggtggatcacttgaggtcaggatttcgagaccagcctgatcaacatggtgaaaccccgtctctactaaaactaaaaaaaaaaaaaaaaattagccaggcatgatggcgcatgcctgtaatcccagctacttgggaggctaaggtaggtgaatctcttgaatctgggaggtggaggttgcagtgagccaagatcatgccattgcactccagcctgggcaacaagagtgaaactgtctcggggggaaaaaaaaagcagaaagaagaaaaacatgataaaaaagatttttaaaaaatgatgatggatgccaaacacacctctaatcctagaactttgagaagccaaggcgggagcattgcttgagcctaggagttcaagaccagcttggacaacatagggagattctgtctctgcctcggccctccctgttctaccctccaaaaaagccaagtgtggtggcacatgcctatagtctcttacttgggaggctgagacaggaggattgcttggaccccagaggtggaagctgcagtgagctgcgatcatgccacttaactccagcaaaaagagcaagaccctgtcacacacacacaacaaaaagatgacagaagtttggtttttttcttcctcttcaatgggggtatactgcatttatattctccttaagatgatcttctctgacctaaccctagaccatgtatttgtccccatgtatgtcctcactgtcccttctttacccactccctggcttgtcaccctctccatattttcctttttcttcctgttgccctgccagtatgtcttgctcatctctgttcccctctcagtcgataaaccagttcagttggggcttgtttttcaggcaaagaaactggtgatagcagggtctggggctgaggggtctttcagccatggtggggggatgtggcccccctttgaacagattactctcaaactgtgttacatgttctttccatgggtattggtagaattgactaattagattgtactaatttccttcaatgtatattgactgccccaccactcattgaaacgctaatggttttttttccctttcttttcttttccttccttcctttctttctcactctttctttctttctcactctttctttctttctttctttctttctttctttctttctttctttctttctttctttctctttctttctctctctctctctttctctctctctcatctgtctgtctcttttcttttcttttctttttttttcttttctttcttggttttgctctgttgcccagactggattccagtgggatgattactgcttactgcagccttgagctcctgggctcaagcgatctttccacctcagcctcctgagtagctgggactacaggcatgtgccaccatgcctggttaatttttaaatttttttgtagacaggatctcactacgttgcccagtctagtcttgaactcctgggctcatgtgatcttgccgccttggtcttctaaagtgctgggattacaggcatgagccaccctacctggcctacggagaactttctgtataggttttcaggcttgtattccagccatgaaaaatcaggtgaaacacaaacaaaagcctcagaaggcagtgaaactgtcagattataaagaacatacaggttgttgattttggatggacattttcatctccttaaatttcatttcttttacagaattagagaaggaatgtgactttggtagtcacccatcatgtttacgaggaaggaggatattagccagtccttggatcatcccctcagaaacagagattattggttttagattgtgcagtcattaaagtagatatgttgttactaagcaactctggccaacagtattggaaaaatacaggcaattttctgcttcaagtgattaaacatactcatcctagttgcctgttgcagaaatacccagtttaattggaagtttcattacctcacaaactcaactctggggtagggtcatgaaccaggaagtaagcaaaacataaaataaataagatagataatacttactttatatgcttattcagaacgtttttactctgactttacttacagcactcaaaagccacatcctagtctaaaacactagtaggtaatggtgtgacattctgatgagaataaagagatacctgatagcctgacagccaggggcaagatggatcaggaagtacccgaatccgaaaagtgtggccatttggggactctgtgagaggagcatccctgtccaccccgggtcattgacactgggtaagagccatattgttgtgaggaatttgcatcgtgaaggtgatgaactgcggtttgcatcttttgttttatttatttatttttttaatttttgagaaggagtctcactgtcaccctggctggagtggcgcaatcttggctcactgcaacccctgcctctcgggttcaagcgattctcctgcctcagcctccccagtagctgggattgcaggggtgtgcgccaccatgcccagctaatttttgtatttttagtagagacggggtttcaccatgttggccaggctggtctcgaactcctgatctaaggtgatccgccctccctggcctcccaaagtgctaggattacaggcatgagccaccgtgcccagccgcatcttctgttttaaaaggactattagagaggggggaaagacatctgttaaaatgaagattggattaatgcatttaatccaaaaagggatattcatctcaccagtctggaaaactcagttccatgaaaaggtcattctctccttccacctccctttgatggtgatatttatgtggccacactgattaaaataatcaagaaagaagaaacagtttatatttttcaaatgaagctagatattacatgatttatttttaagaaattgaatacaattaatgttttgttctagatagtacagttttcttgtaatctcaaatgtatttccttgcctgccttcagtagtaagcagctatcaaatggtgccctgctctgatggtgtttacttccaccattcacagtattgttcttaatggcatgcggattctagagtaaccaataattagatactcagatgttttccagtttttagtaattagatagttagatgtcttctagtttttgtatattacaaatagcattttaatggacatgattaaagctaaacatttggacagatccttcatttcctagggcgtacaaatttcaaggcttttgctatgctagattgatttcctccccaagagttgttaattacttacattttccccggtgaaatgagagagctccctacaagtccagatttgatgttatattttaataatcactgtcaatctcaccaattttcataggtgaaaaataatgcattgttgttttaatttacatggctttgtgtaatagtggatttcagcatttgcatttccttggccatttgtgctggttttttttttttttttttttttttgtgaatttcctgcacacattctttgctccttttaaaaaataattggtaggcatttataattcatatcgatatgtaggggccgttgatttattaaagatattttgggtctgggatgtgtactacaggttttgatttcagtttgttttagttgccttttatttgttctcatggtagaactattttgttttcacagggaaaacaaatttaatctaatggtgtccttttataacctgtcaggttaatgaagcatagaatcaaaatatttgcagaacgatgatacattaatttacagattttttctgtacattggatgtgaactcttttaaactataacatggacactttattggaaccaaactttagttttctacattaatgagaaacactggtaactttgcttcattgaaaaaataattgcttgttctctgtcagatgtaagtagtttgagtttatttttatcttctcatctacaaaataggaataattcttacttcatagttgttttcttgttcagttgtctggcttacagtaggtgcttactaaatggtagcttgatttatgatcataatgacataaatgaatatgacttggtctatatattctggtagctgctgagggtaacacagatatatgattacactaattgtacttctgaggatgtgagcagtgttttgttgttgttgtttgtttgtttctttggtgttgattttaggtagaactttgattatcaattttatgccttctttttaaatgcaaagtccttaaagccaaaagtttactctaagacttgctttagctataactcgcaaattattatcatttactttaaaatatttcagatttcccttatgatgtcatctttgacacatgcttatttagaaatattgttaatattcagatattttgagttgttagagatgtcttattgttgatctgtaatttaattctgctgtcaccagaatacattctgtgtaacatttttatgttcagatattgattgaaatttactgacggcccaacataaggactattttaccttaaaagttagaagtatgcaacagtaaaattctattaccttccccatcttgtgtgctattgtcgtatattttacttctgtctccaaaatgcaccattaccatttttgctctaagcagctagttgtttttcaaagtaatttgtcttctttttatttattttttatttttttttagacaaggcctcactttgtcaccccggctggagtggagtggtgtgatcttggctcactgcaacctccgcctcgtgggttcaagcgattctcccacgtcagcccccgagtagctgggtctacaagtgtgtggcaccatgcctggagaattgttatattttttgtggagcccaggctggtcgcgaactcctgagctcaagcaatttgactgcctcagcctcccaaaatgttgggataacaggcatgagctatcatgcccagcctgtccttatatttactcacattggtcattttggtttctctttattctttctgtagatctgagtttccatttgataccatctccttatagcttgaaaagtattctttacgctgttttttacagttcaggttcattgctgataaagtctctcagcctttttctgaaaatgtcctaatttcaccctcatattttaaaggacattttgtgttggtgatagaattcagagttgacaggtgtttttttttttttttttttgaattggagtctcacctatcacccaggctggagtggagtggcgcgatctcggctcactgcaacctctgcctcccaggttcaagtgattttcctgcctgagcctccggagtggctggaattacaggtgcccaccactgtgcccggctaatttttgtatttttagtagaggcggggtctcaccatgttggccaggctggtctcgaagtcctgggctcgagtgatctgcccgcctcaaccacccaaagtgctgggaatacaggcatgagccaccgcacctggccagttatttttgtctgtctttcagcactgtaaatattgttctgttgtcttcttgttttcattgtttctggtgtgtcagcttctgcttgtttttgttgtttctctgtgtttaatgtgtgtctaggtatagatctctttgtttttgtccttcattgaacttgctaagattcttcattatgtaaattaatatttgctaccaactttggtaaaattccagtcattatttgttcaaatattttttcttcccctatttcttcttcttttaggactccaattacatgaatattttattgcttaatattcttccataaggaccttcattcagtctttgttctttttcttaactctttttttctgtttgttcttcagatttcagttgtaaactatagaaggaagggttagcgactctgggaataaatcaatggaaagtatctgctattaatgatatctaatttatttttcatttcaggtattaccttttcagtttgagaagttctatttggttctttttgatattacctatctcgctcctaaactcacatctacttgttataaccatatttttctttaactccttgacatattataataggtgcttcatagttgttgtttactaattccaaaatctggatcatttcagggtctatttctgttgccatttccctcctttgattacaggtcacattttattgtttccttgtgtatctagtaattgttttactatgttgaacattatgatcgatatactatagggagtctggattaaagagcattgagttttgttcatgcaggtagttaaattcctggcagattggcccctttagcattgaatccttctgtctgtctgtcaatcagttaatcatagggcaggtagctccattctgaacttattccaagtgcacagctgttaatctagggtgagatctttattactaagacatagccttcctggtgtctcaggtgaatgcctgaggcccagtggagatttccttcctctcctcgggcggcatctcctactgcatgacctccagaatcttcggtcagcactcagccccatagctgctgctttcctaggcctcatggagtctggccctgcttatgaagccgttggcaaagtaccctgggtaaatccctactgaaacttctggggcaaaccctctgctactcaggctcacaaattccagatgcttctacaacttggaacaaccatctcttcctccaccgctcagtgaaactgttttgcttgggcagaaaatgtcctcaggcataaagccaggtcagtcccgggccaccacctacatttcccgtctcttgaggctcacagcctgtgttacctgtggtccagtgcctgaaaacagctgtgtcatatgttttgcccaacgttacagttttccaggggagggcaagtcctctctcatggttggaagcagaagcctggtagtttgcttgtcagctgtgtcttcctctttttttttttttaagatggaatcttgctcagtcgccgaagctggagtgtaatggcacgatcttggctcactgcagcctctgcctcctgggttcaagtgattctcctgcctcagcctcctgagtagctgggattacaggcacctgccatcatgcctggctaatttttgtatttttgtagagacagggattcaccgtgttgaccaggctggtctcgaactcccgacctcaggtgatctgcctgcctcagcctcccaaagtgctgggattacaggtgtgagccaccgcacctggcatgtcttcctcattaataatattcatggcccttcatgattttttttatcctatctggagtatgactgatttaatttcagcaggcccttaatttctgtgttgactgtagagttatcgttcacttgggttaacttctattacaacccgagtatctgcccagccttttattccacctctacacctggtcacagtaatacacgacttacaagtagccctcctggtgcacccagaagaactttgtctttttcacaaattggcatctgtgctgtcttacctaaatcctcattgttggcctcagacacaatctgtggatgatagttcaagttgggcacaattgacttcagatttcttgaaaagggtcagcttaggcttagagtttggtgttgacattttgcttttatttttttaaaattaggtatcaaatttacatgctttaaaaatgtggtattttctgcatgtgggattaacattttgtatgtaagactggctttataaatatttacagatagtttgtagtcaataaggcacattacgttctgtttctttcctacaggggcgggctggtctctctccttccctttcttctccctcactcctacattttcttctttctttgtattttacgattgattcacttatatttgcataatagcattttttaatttttttttttttgagacagtctcactctgtcacccagactggagtacagtggtgcaatcttggctgattgcaacccctgcctcccggttcaagtgattttctgcctcagcctcccgagtagctgggattacaggtgcacaccaccacaccaagctaatttttgtatttttagtagagacagggtttcaccatgttggccaggctgatctcgaactcctgacctcaagtgatctgccttcagccccactaagtgctgggattacaggtgtgagccaccgcgcccggccattttttaacttttcaaaatgttttcacttctatctggccctttttttcccattcacatagccctcgttgacagtcttgttgccttttgcctggactgttattccagcggcaactcattctcctccttccagtagtctcctggccaattgcttctactgtttaaccttccttaagttgagtttctccctctaacttgaaaaaccacacgtactgttccatattgccttccataagcttcacctgcttcagccctgcttccatgagcttcacctgcttcagccccatttccataagcttcacctgtttcagccccgcttccatgagcttcacctgcttcagccaggtttggggtgttctttattcagcagcctccacatcctccttctccgtacttctgtcctagctaggcctcccctgcagtgcccttcccttccctccataaagtccctccttcatcgtccagggtctctcccacccagtgtccctcagttcgaactgtccccagttctctgtgcaggtcacagttgaatcacaggtgtctggacatacctagccctgtctcccaaggcaggaaaacatgactgtgtcactgtatctgctggggctcctggtgtcctccagatagttgatactccatatttgtcatttcccattgaacacattgaaaccctgtgatccaggcagggtgaagagactaaggccccatgtgtttaataatgttgcacagagaattagtgattggtacggatttatagtccacgtcctctcattttgttcctctgacagcctctctgttcttccctaatttggctcaaaattactttggttggctcttaagtagaaacttatgtccttttgtacttgggggcgtgtccaaattcattaaatgaggtgggagatttttcttcttcctacctctcgctttgctgtcctgacacagctgccaccactaaaggtgctggtggggcagagagagggggcagtgggggggtggggatgcttgtgcttagatctggtcttgacatcacacgagacttcctcaggtctgctgtgtcctaagcctgacttagtatcactgcatgaatacaattctctacttacctcttttatttcaggcacttcgtcttagtaacatggccatggggaagacaaccacaggccagatagtcaatctgctgtccaatgatgtgaacaagtttgatcaggtaagctgcagctcagggatcctttttcattgcattaaacacccctctttccccttttattctttctggaggcaggggctcactctgttcacactggattgcagtggcaggatcatagctcgctgaaggcttgaactcctgggctcaagtgatgtgcgatcctcctgtctcagcctcctgattagctgtgactacaggtgtgcatcaccgtgaacagctaatttctttttttttgaattttcatgtcagatgggctattgtggaaacatcataacaaaattcaagggtggcacatctcacacaagtgcgtgaacacccgattatcatgctcatgaactacaaaaggattgctggctaatttctttttttattatttgtagagaagggatcacgccttgttgcgcaggctggtctcaagattctggcctcaagcgattctcgctaccttggcctcccaaagtgctgggattacaggtgtgaacaccatacccgcccagtatccattgtttaatacttaccttgtagcggtatcatttttccagggcattctagctgctggccatattcattttgcactaggaccacttcttttttccaattattcattcatcagaatgaactaaagcctccgctcagtgtcagggacaatgaataagacttggtttctgttctgaagaagctcactgcctgtctgggagacaaacacacaagtaaattactggagtatgtggtgggcactgtagtacaggttttcttggggtactggggagccccagggcatatgggagctgaattctgtggcagagagttctggaaaatgctcagctcatcagtgaagatagtcacgatcaagacattcttctaatgccgctggtcggattctcttgcgagtctcccagacccactctttgtttccccagaatggcttcacctgtggtgggtgcttggctttctgctttagcaggtgtaaacccactgtggccatttctgtttaggatcttgcctgttcttttccagtggatcgtttctgtactgtgttcttactggttttgcttttctggagtcctaagaacgctctgtctttttctttaaaggtgacagtgttcttacacttcctgtgggcaggaccactgcaggcgattgcagtgactgccctactctggatggagataggaatatcgtgccttgctgggatggcagttctaatcattctcctgcccttgcaaagctgttttgggaagttgttctcatcactgaggtaagagaaatactgctgtgaaaaatgtcatatgtatttggtaacatccacagtcttcccaatgcttttgttcagaaacaaactaaatgccttttattatctcttccttgcatatgttgcagctcttcaggctcagccaagggggcctctgtccttatgctgaaggcaaatcttcaagggagaaaggtgaaggcattcactggctcctcccagccagcttttcaggcgctgtggcctcaataagtagagtgtccttaagcagaaatcacatatgtagaagttagttaaacatataatgaataatgataaattgaaatgtttcataggctccattctagctgcatacatctttaccgtgcttgtgttagagctgttttcacgtgtatatatgtgtgttatttttaattcagtatttccattaaattgtaagctccccaaaggcaaggtgaattttaatgatgtagttataaaatcacacagtcctagtagcatatgtgttaggaatatggttgttagctggtcgtggtggctgaagcctgtaatcccaacactttgggaggccaaggcaggtggatcacttgaggccaggagtttgagacctgcctggccaacatgacaaaactccgtctctactaaatttaaaaaattagctgggcatggtggtacacgcctgtaatcccagctacttgggaggctcaggcacgagaatcacttgaatccaggaggcagaggttgcagtgagccgatattatgccactgcactccagcctgggtgacagagtgagactctgtctcaaaaaaaaaaaaaaaaaaaaaaaaaaaaaagaaatacagcttttgatcaagagattaatgatgataaaataaattagacttgaagcaggttccaacgaattggaaatggttcattgtcaaggttgtcagctggatcgagatgagccactgttggaagtgcattgagttctttgttttatgcttttgcttatggtgtattttctacttggaccttcccctctttgccatctggagaaatagtccttgtcctttgaggccccgttcaaatgctcctggtcctctgaagcttttttttttttttttgctctcctctttcccggtggagttaacaaacagccctttcttttctgctccccagcacttcattcccattttagcactctctgaccactgtgttatcgtcaggctcgggtgcctttctgcctttcctgcccacctgtgagctcctcgaggggacagatgacatctgactcattttcatgtcttcctcagcagatattacaaattctccaaaatacttgttgagttggtatagattgttgacttcctaatcgaagtctttttctatttgtattttgagtaggacctgtctgtgttgtcggaagcaaaagcgctgtagggaaccatgtgatgcaggtccctgcctgtgcctgccgctgataagggcaggctgtgatcgctcaggctcatctcccgtgtctggttccccttaggagtaaaactgcaactttcacggatgccaggatcaggaccatgaatgaagttataactggtataaggataataaaaatgtacgcctgggaaaagtcattttcaaatcttattaccaatttgagaaagtaagtttttgtatacattgtaccatatttttgtatttttctatttgctattcttgttgatggttttagaagttttactatgttttcacattatgggaagttcagtacaatccatcccagtccccacgcttggcagttgggttaagtagaatggtagagaacgttattttcacttggtcttccatttaccatgttcttaaacagaaagcatgtatgcttcttactaatacaaaagtgaaattccaaatgtttctgaatatcccaaatcccaacaatgagtctaagatgttagtatttgagaataatggtgtttaaaataaaactataatttatattaatgtagccaaatggacaagttatagagactgggcatatctatattggaccaaactatatgaatcagtatttttcattccaaacaggcatggaaatactgttagttgtgctttgttttattagaaattttggagggggctgggcgcagtggctcatgcctgtaatccaagaactttgggaggctgaggcgggtggatcacctgaggtcaggagtttgagaccagcctggccaacatggtgaaaccctgtctctactaaaaatacaaacatttgctgggcgtggtggcacatgcctgtaatcctagctacttgagaggctgaggcaggagaatcgcttgaaccaggaggcggaggttgcagtgagctgagatcgcaccattgcctgggcgacaagagtgaaactctgtctcaaaaaacaaaatatgtattttttttttggcttttggattaaatatttgattagttgttgcattccagcacatgatacttcttatgatttcttgggtagttcctggccacctctcacctttgcacttttttgttgttgttgcccaggatggtctcaaactcctgggttcaaacaattcatccacctcagctcccaaagtgctgagattgcaggcgtgagccactttatctggttgacatcactgcttttttaaaaacctgtactctctttcaggaaggagatttccaagattctgagaagttcctgcctcagagggatgaatttggcttcatttttcagtgcaagcaaaatcatcgtgtttgtgaccttcaccacctacgtgctcctcggcagtgtgatcacagccagccgcgtgttcgtggcagtgacgctgtatggggctgtgcggctgacggttaccctcttcttcccctcagccattgagagggtgtcagaggcaatcgtcagcatccgaagaatccaggttggtgtcagatgccattttaaagttgtaggtgcatttgaagtagaatgatgtctccttttatttagcatcactatgttccagtgtaatgacatttaactctctcataaccaaaacgtgccgccattccagaatgttgtgtgtacttccctatcttagatagagcgcggtaaagacaatgcaagcattgtgattcatttgttggcagcaacataaacagcagtggcacagggcttttgatgtacaggtggtaactgcaggggtgattaaaatgtctaagctgtctaaaagcaggaaaagcaaatagatttcatctcttatgttaacaccaatgagaaagtagtgagtatgagttgagtatgattcttctgtgggctggaaggaaagagcttgctgggggtctctgcttcttggtcaggaagaatggcctcaacttcacttagctatccctgtttgaaaataagttctttcctctgtagatgttttccttgttaatttctgtgcatgcctttaggacctctgccatttctttcctgtccgcttttcacttttttcttactgttgatgaacctatctctgccagacttctttatttccaattggttgatttctactgccttgctccatttgtacgactgtaacaaggaccactctctgcttcccagatggacctctgtgtcctcccatcgtggaggaaacagaaggacaaaatgggcctggttagttctctccagccttctgtaaggacactaatccccttcaggagggctctgccgtagcgacctgatcacctcttagaggccccacctcttaacactgatgctggggtgaacttccaacctgtgaatttagaacaggcatgcccattcaaaccatagcatccactttacacaccatcaccttttcctaagtcgctgtcactcaattgccaggggcaaataatttatatcaccaaggtaaggtcctggcacataatgtatgctcaagtatttgtgtgtttatgaataagttgaaaattaaaataaattggctttgtgagagggcttggcaagaacttgactgtctaagcttttcaaaaatctgctttgttttattcctggttcttaatagagatggatgcccaaggacagactcgagtgaacttagctgacactggctgtgattatttttttatttttatttttttggtaggggagagaatggggtgggtgttgtcataaaagctaagattcttttttatgatgaagtttgttttttaaagatgagatcttgctgtgttacccaggctggagcatagtggctattcataggcagactcccactactgattagcaagtttgtatttcataaaaaatattacctataaaacagaccatacagggtcatttcttatccccattctttctgacaacagcttcaggtaacactgtgaagggatggtccatgcaggtcagtaacacttttttcatgtagcttttgcatcttgaaagaaagaacttaggagtatactttgaccagagtatctttctgagtgtgcaggacacctttttcagacggaagggccacatgtgtattgtgctgctgtaattctctgactgctgttgctgcccctcaccgtgattgcaggtgttggtgtgtgtaccgtggaccagggctcttggaagcctgtacatgttatttcatgttatcctcacaattgaaggagataagcattagtgtccccactttacagagaatgaaactggctcctgtaataggttaaggagtttgcctatcttagaactaggaatctgttcagaaccatgatttcttaaccatggtatgttgcctgttgtattaatctgtttttatattgctataaagaacttcctgagactgggcaatttatattggaaagaggtttaattgactcacagttcagcatagttggggagacctcaggaaactcacagtcatggcggaaggggaagcaggcactttcttcacagggtggcaggaaggagaagtgcaagcagggaaaatgccagacgcgtataaaaccatcagatctcatgagaactcactatcactagaacagcacaggggaaactgcccccatgatccgattacctccacctggtcccacccttgacacgtgaggattatggcgattacagttcaagctgagattcgggtggggacacagagccaaactgtatcacctatgttgctgccttagtatcctctggtgtgggatatttccttcagtattcttttactcatctagttgtattccaaaaaccttgaaaggttctctgtttcttgtggagtcctatttttgaaccctaggtgtagggcttacctgtgtgtgtttcccagcacatgactcgtctctgttcccactttggggtcttttgctctgtgtggctggggtgtcctcccttctgtccttgatttaagcaaatgctactaagccctccggccaggtctcaggagcctggtggtacttttgttcctgccgagcaaggccagtacggtgcagctcagcattgatggacacataatgggcaccaaatgctggatggacagaagcagcaaccttagcaaaagtctggggcccaatacagacccaaggtggcccagcctgcactagaagctagaggaaggccgagcgaggaagcagactcgagttgaatattcccagaacaccaggccttgttcaaggggggtgtgagggtccttaactctggggagctggatttttttcagagtggactggatcggagtctctgcctcgttatgtgtgtccctctgtgcatctcaaaccctcttggacttggtggacctgctcttgtaagagaggggccatgtctgacattaaatatagaccctcctggccaggcgcggtggcttacagctataatcgcagcactttgggaggccgaggtgagcggatcacttgaggtcaggagttagagaccagcttggccaacatggtgaaatcccgtctctactaaaaatataaaaattagtcaggcatggtggtacatgcctgtaatcccagctcctgagctacacgcctgtaatcccagctcctcaggaggctcaggcttgcttgaacccaaaaagtggaggttgcagtgagccgagattacgccactgcactgtagtctgggtgacagagtgaggctctgtctccaaaaaaaaaaaaaaaaagagcctcctgactggcttccaggctgtgaaacaaatggactttccttgggccatgtggagaaatagaaacagtcttacttggttaataaaattagtctgtcttcctggccattatgagataaatgaagattaagatcaagaccatggggctttatatgaacaggtaatagctggtgagtacccactaggtaacaaggaggggcccaagtgccttatgaatactcactccttttacttgtgtaactcccctatcattcccacattacagataaagaaactgaagcctgaggagggtgaatagcacctccacgttttaacagttaataaaaggctgatccggggctttagcccaggccgtcaatctgtctcagaagtgcggccaggcgcgatggctcacgtctgtaatcccagcacttttggaggcctaggtggacggatcacttgaggtcaggagttctagaccagcctggccagcatggtgaaaccccgcctctgcaaaaaataaaacaacaacaacaaaatcagctgggcatggtggcacgcacctgtaatcccagctactctggaggccaaggcacgagaattgcttgaacccgggaggtggaggttgcagtgagctgagattgcaccactgcactccagcctgggtgacagaggagactccatctcaaaaaaaaaaaaaaaaaaaaaaagtgcacattcttatccccacatgcacagcataacctgaatgcagatctcacttccatgcggcagccataaaaaataattgggcaaagagcagccccaacaggtggtatggcggggtgggacgcagaccttgggttctcaccaactctctgggccttggttttattgtctttaaataaggggattggacttagtgtctggaggtcatttcctattttgtctcacttatttagaaaaccgtctgtattagtcagagttctccagagaagcagaactgatagggcccacgtgtatgtacatgcagagagagattggtagattgattgataaaattgtctcacgcaacttggaggtgacgtaagtctgcaatctgcagggcagctggcaggccagagaccctgggaagagctgatgatgcagcttgagtctgaggaggacagtcttgaggcagaattcccttttctttgtaggtcttttttctcttaaatgccttcaactgattggatgagccccgctgacattatgtaggattttaaagtctacagatttaaatgctaatctcaactgaaaaacactttcccattgatacccagacatgtttgaccacatatctggggactgtggcctagccaagctgacacataaaattcaccatcacagtcttttttttcttttgagatgagtctcactctgtcggctaggctggagtgcagtggcacgatctcagctcactgcaacctctacttcccgggttcaagcaattctcctgcctcagcctcctgagtagttgggattacaggcgtgagccaccatgtccagctaatttttgtatttttagtagaggcaaggtttcaccacgttggccaggcaggtctcgaattcctgacctcaagtgatccacccgccttggcatcccaaagtgctgggattacaggtataagccaccgcacccggcccatcacacactctttaatcctcatctcttcttttgggctctccatacgtgactttgtcctcattttccccttattattgaagccatttgtttatagatttctttcccttcctaaactctaaacttacaaaaggcagacacagtgcctcattcatctttatattacgtatagattgcaggtgctcagaagatgtgtaagtgaagtcaacctccagcttcattacatctatgagatttggaaaatgcctaacaactgagcattattttagtgggaagtgatgactgtcgttctctgctgaattatgtcacacatcagagagtgtggcatgaaagggatcctgcagacgattttcagggaattgtggtccaatatgcagctagggaagaaatccttaggattttttatttttttcccaccaaaatgcaactcacctgtgtctaaggcttgaaattcctgttcctgtagatagatgtatgcatgtagtaaaataaaatgaccggctgggaattttacattttccagtggctaactttggcaaggtgctctgtgtccagataaaggatgcatctttgacttgggcaggaaaccttaatttcccctcatgtacatagtttctggtgggagggtgggcctagagttcagcagtagtgattttcagtaaaggagaaacaaatggccaggcatgactggtaagaaagcgttggcgctcgtatggtaaactccttcatggtggtgttgaagtcattgttggaggttttgttttgttttgtttatttgtttgttttttcttcctttagaactttgtgttttttcaacctggatggttcctgtaacccatttaaaattttgtccccaagttcatcagaattacctgatgcactgattaaaactaaattattacaccttggagattctgcttcatcaggcattggctgggtcccaatatcttttgttttgttttgttttgttttgagacggcgtctcgctctgtcaccaagctggagtgcagtggcgtgatctcagctcactgcaacctctgcctcccaggttcaagtgattctcctgcctcagcctttggagtagctgggactacaggcatgcaccaccacacccggctaacttttgtatttttagtagagactgggtttcaccacgttggccaggatagtctcagtctcttgacctcgtgatccacccgcgtcggcctcccaaagtgttgggattacagcgtgagccattgcgctcagcccaatcaatatctttagaaagtgtcctcgtgtccctgaaaatatgctgtctttgggaaaccttgccctagttgtgtgataaaaaaacgcatgaggtcaaaatatgtgtagcaaatagaaacgacgtcaggataacttaagactaaaatcatttgagaggaagcacgcagacacctgttttggtctatgctaggcttttgggggttaagtttggaatttattcattcacttagtcattgactctacaatgtaattgagcacctagcttaagctggcactgtgataaatgcttagatacaatggtaagccaatgcacagaagtccttcctgctgggaacttcctcctgggaatcatcaagtgggggttaataaataaccacgtaattaaaatcagtgctcatgtgtcattgtctttggaaggcaatatctgagatgccatgaggtgctttggtagagatgcccatgccattgtggaggtgtagtgcttcctgcgaaggctcaagaaatagcctgcagcattgtgtgcgtggtagtggagagagatggggaagggtttgggaagggaggtggcttggggcagccaggaggctgatgccctgcatttccctgggggtaattatgtgagaaagctcttaatgttcaagaaaatccacatattttcaattcaccctggctgcagcctggggaatgcattgggggcccagagtagaggcagggagatacgtcaggaggctgatgcagcaagcagtcccggaagatgatgatgggatttggactcagaccaagtagacagccttggatagatttgaaagccattgagagcatgtcctgatttgggtgtggggcaagaggagggattagtcagaaggagggatcagggctactttcgtccttatggctgcaaaatcacctgggttcactgaaatatggaatactagaagaccagatttggggatgaatttggctttgaagactgggatgctttagaactgtgcagatgtttgctacaagacccactgatggggtctgcctggttccgcctcctttcctatttctctaaaaatggaggttgtaaatgatgagtgtaatgaattgatccaatgttcactttcttccttgcctctcgtggatgtgttaatgaacaatgagaaggtgaaaaatggcatcaggagaggaggaatagttctgcccaatatactgatcggaactaagtgagtcttgaccatagatagaccagagcttggcagacacatagagtttagaaaacattgtgttgttaaatttttaaaaaatatttttatatttctattttaatttttttattatactttttctgggacacatgtgcagaacatgaaggtttgttacataggtatacatgtgccatggtggtttgctgcacccatcaacccatcatctaggttttaagccccacatgcattcagtatttgtcctaatgctctccttccccttgcccctcaccctctgacaggccctggtgtgtgatgttccccttcctgtgtccatgtgttctcattgttcaactctcacttatgagtgagaacatgcagggtttggttttctgttcctgtgttagtttgctgagaatgatggtttccagcttcatccatgtccctgtaaaggacatgaactcatccttttttatggctgcatagtattccacagtgtatatatgacacattttctttatccagtctgtcattgatgggcatttgggttggttccaagtctttgctgttgtaaatagtgctgcagtaaacatacgtgtgcatgtgtctttatagtcgaatgatttataatcctttgggtatatacccagtaatgggattgctgggtccaatggtatttctggttctagttccttgaggaattgccacgttgtctttcacagtggttgaactaatttacactcccaccaacaatgtaaaagtgttcctatttctccacatcctctccagcatctgttgtttcctgactttttaatgatcaccattctaactggtgtgagatggtatctcattgtggttttgatttgcatttctttaatgaccagtgataatgagctttttttcatatgtttgttggctgcatacatgtcttcttttgagaagtgtctgttcatatccttcgctcactttttgatggggttgttttttttcttgtaaatttgtttaagttccttgtagattctggatgttagccctctgtcacatggatagattgcaaaaattttcttccattctgtaggttgcctgttcactctgatgagagtttcttttgctgtggagaagctctttagtttaattagatcccatttgtcaattttggcttttgttgccattgtttttggtgttttagtcgtgaagtctttgccatgcctatgtcctgaatggtattacctaggctttcttctagggtttttatggtttgaggttttatatttaagtctttaatctattgtctttcttactcatacttttcaatttccttttttccatgtacagaaaatgaaggttttattgcgaagtttttaggtctaagaagttccgtctgtttttcttttattaaggaaaggattgatggtcttggcattaagcaagtccaataggattttaagagatcttatcttaacagcaaagcactgaaagtcttcaatgacttccacatccagtaaaatgtgtctctgaggttttattgttcacaaagctgcttcagtttttgccaaccgtccactctgaagtcaatctctttgacttcagttctgccgtctgatacttccccaagtgaactttctggggatgcacctcattgctgtattggcaatgtcatttcagctttttcaccctcctgagtgactagagaaagtccagctagcccctccagcatctttacaagccgttcatgtgttgagtggagtctcatttaaccactaaactattatcatgtctgtctccactcagatctgttgtttattgaccagtgctcaataggtttactgtgtgcatagttttttattttgaaggtggggaacttctgttctgacctgcacatacagaatactgaatttgatgtcttggtcgtacacttcagctaaaatcagaattttattctgtctagacaaggttaagaagatgtatatataaccttttgcttgttgctgtgctaactgacatagctgattgtttgaataatttaagccgatcatacagtctgtttatgactgtagcatgtatctgtgaagagctgaccccgtgtgcagtgcagtgttcatgcaggtgagtgtgtttgtaagtgcaggatgggggacacatctctgccctgtacataactcagtttcggtcccagctgcagccaccctgagctggttcccagcaggattagtctttgaggtgggccctcaaagacagggtgggattaatactcagggaatcaaggcaaaggctactttgggagtgtgactgtcagtgctgctgggtcatggaagagtttgaagcatggtggttattatcagaggtgttttcagaagatccttagggagtggtgtttgggaacggtttggttgtgatgggggatgctggagaagggaccagtgaaaacattgtatcagtatcacaaatgattgccggtcagtaaacttgtgaaattgggtctggtcttttttctttctttccttccttccctccctcccttcttccttccttccttccttccttccttccttccttccttccttccttccttccttccttccttccttccttccttccttccttccttccttccttccttccttccttccttccttccttccttccttccttccttccttccttccttcctcctttcctcctttcctcctttccttttattttttttgacagggtcttgcactattgcccaggctggagtgcaatgagggaggagccctcatggcctaaatacctctcaataccatcacattggccattaagtttcaccacctggattttggaggggaagttgtccaggcagcttccaaggcctggagactgtataggaagtttcctggacgttgtcactttctgttgggctgaacttaaagtcatgtggctacatctaactgtgttgggggctggagagagtggtccttgttcaaggctgctgtgcgttggctaaaaatgcacagaaaattagagcaggttttgaggagagctgacagtttttgccacatctacaaaattttggatggggtgggggtgatctaggttgtttattctgtttctaagccactgtagcttctctcactgaattatgcctaccatttgaggagcaaaagggagttttcccatatatcattatgttaggtttgttatggttaacaggactttgttttattcagttaaattgcattgtgtagttgctaactagtgtggaaaatagcaataagcgagagattggtccgttctgctgtctaggaggggacattgactaaggaaaaaagataagtgtaggtgtagcatggtggcttatgcctgtaaacccagcattttgggaggccgaggtgggtagattgcttgaggccagcagttcgagaccagcctggctaacatggtgaaaccctgtctccagtgtaaatacaaaaattagccaggcatggtggcagacacctgtaatcccagctacttgggaggctgaggctggagaatcacttgaaccggagaggcggaggctgcagcgaaccgagatcgtgccatgctttccagcctgggtgacagagtgagactctgtctcaaaataataataataacaataataataggtgattgtgaaaagaagcttataaaatatgttttaagagatagaaaccttttttcttattttggtaaagaacactgacttaagtgatagtgaattaagttgtatgtcaatttgacgtgcacgagggaggagaaggccagttgaatctaagtgactggccagagccagggctgccttcagaggctctgccctgtgtttcctgcctacccttctgggctcacttgcttcctgaaggctgggaatggccttgactttcatgttgccagcatctagcgcggggcttggcacatgatgaggattcatgaaatattcatcagatcatttccaaccaactcccgtgttcaccattagtaacacaaaagtacaacagtagaactatgcaggctgtcaatgcactcagtctcagagcagtattttcggtttatttaggaacaagtatacttgttgaaaaaaattacacagatagttcctaaaattacaaagatagttcatggccatgaactctttacatgtttatttattaaataaatatcatatgctgatgtgaaccgatactttaaatagggttggtaaaatgatctgtaggctgtataaagttttatgttacaaattccatggaagcagtgacttttgtttgtgcttttgtttctgtaatttaaacaatctgtgtttttcccattttctttagacctttttgctacttgatgagatatcacagcgcaaccgtcagctgccgtcagatggtaaaaagatggtgcatgtgcaggattttactgctttttgggataaggtaacatatccttaatgcaagatcatgactgctgaagataatgagtgacattgatttaatagagaattttgaggtgttacacatggtataaaatatggcttgcttatttactgaaactatgttacaaagattctcaaaattaggagtaagttttgcaattaatagaaagtaggtatgaaatcagacttaggtatttgacaagttgtattttatctcattttcaaaaaaccaaggaaagtgttacctcgttctaaactaaatgtcactaagtttggacattggcaacgctggattttataggattggacagactaggggtgcagcatggaatgggttaactgctgagtacattgtagattattacggagactttggggtatcagtgcacattttcagcttagcgttttcaggtcatcttattacatattgagaaagcccatgacttggatttttgccacggggttggggtggggctcccctattccaagcatctggatgttgccagctgctttggtggcagcccaaggccgaggggaggctgcaggccggcgtagcaagctccatggattcctgactgaagggtttcaccgtgtggtaagtggacaggtagttctggcaaggccacaaggatttttcaacacttggtttactcataacatttaggcaaatgggaccaacagcaggggccacccacaagaagtactgaggctcggtgcatctcctgttatccacaactccagtgccctcctcatgcttccaaggaagcagtgggctccctgcccagtgctcatagacgccactactatggcaccagcttttcatgaagagaaaggctgtattgtgagtggaatggcaaggagacaggaggcaacgctcaaatttgtcttccctagctgggggatggggcaggttttataggcagagggtaatgaggcatggtccgattgggtcttgcatgatgccagagctggacctgatcggatcctggatcatgccatgaggcgttcgcctctgaactcgttcctccttccttggtcctagcacttgggttctgcccatggttgcctgcttggttcatctgggaatgctcagattacgtaacttgcaacctgggatccatggtgactgaaaaactgtttactatgttattacacaaagttgaaccagattgggctggttctgcggttacgctcacctgagtccggggtcctctgcaaatgcacttgtctcagtctgtgttagtctggaaaacagaacacacagcatgggagagggaattttgtgcagggaattcgtttcataggtgatgaaagaaagagcacaaagggaaagcaggggtggaggaggccaccccgagatcagccacggcaggaagctgcaccccggggctggagatgatgtctgccaggagcagagcccagtggcccagctgccagcaagatttgggagcagagggcgagggtctcttctgagggtactggaacagctcaggctgcttaaaacaggtgggcagtgctggtggtgaccagctcctcccctctccgggtgctctttgtggctgaccttgcgttgtagcgtagtttgctggagccaagagccaataggcggttgagcagcactcctgcccagccccactgcctccgtcctcccctgtgggacaaatgtcaaaactctctagactccagactcttcctttctctacatgcccttggctctaagtattgcactccctgtttcatgtttgcctaattctgatgtaatttctgccatctctaccaacaaaacaataatgtgttcccagatatttagaacatcatcgcatcttgtgcacatggaaactcacacaaggtgacagtttctctttatctgctggcattcccaccctcatacactttatttatttatttatttgttttggagacagagtcttgctcttttgcctaggctggagtgcagtgctgtgatttcggctcactgtaacctctgtctcccagggtcaagtgattctcctgcctcaacctcgtgagtagctaggattacaggcacgccccaccatgcctggctaatttttgtatttttagtagagacagggtttctcccatgttggccaggctggtcttgaatttctggcctcaagtgatctgcccgccttggcctcccaaagtgctgggattgcaggcatgagccaccgtgcccagctctaatacacttcaagcattgacagacagctctgtgaacatcccactagaaaaaagcactctaggtgaatgctgttgagctagaggaaggcactgaggtagctaccacacactgtgtaggtcccatttgcaaatttcttttgtaccaaaccctgcgatattgtaaaatggtctgttttgttgtttttcggttcctagataacattgacagctgcattgtaaacagaacactccatcctgttttctggccttgtcttggggtgattttgcctcacactggcagggattattagggaggtcctgcggctgagagggtgctggggtgggaggaggggcacagacacgggcttctggagagaagctgcagaggaaagaagggtgtgctgggctgggatttcggagaaagacagagaccccagcagcagtcctgctggatagaccagcaggtattggagggaaggtcttctcaccagcctaggtctcatttataaaacaggggcttgggctgaagctgtaaagtctgtggccccgtgagagcctgtgactctccatggggaagggatttgcttctgctttcttcactgctctggccccagggcccacagagtgggggctcagtctgcctgccttagtgtgtcttggagcctggggaggtcccagagggaatgggcctagccttctgagctgtcgagtgctgcccccttctggctgcagcccaccagtgctgggtcccaggcctcccggcggggcagaaacctgctctaagaaccagggccttgatgggaagctgggttttgcatgtggctcactcttttgcagccaaaactgaataaaaagaagtaaaatcagaagtagttgttaggaaagagacacactgcaaaggaggggaaagggacaggttgaccggttgtgttttttcatccttttgtaatcacagaaataagtaggtttcagttgtcttaagagacagatgttacttaatcaggaatacagagtagacccgttacattttagaggaggaacaccttatatagtaaggcgaagtcaagcaagactttgtgaatttgtgacaagtttgcacttaaaatcgtgtaaatatgtcacattttgtttaacgttatttttccaagtgtttaggtaatgtattacttttttaaaaaagaaatagttgttaggcatgtatgaaggagtaaagaaacctattctgggtggttgttatataattcctatgaaatctggggctaatttctttgtgcttaaaagggatcatgatcgttttggtctgcggccatcctatgatgtggtatgggacacataaggggtcagttttcatacacacatattttagcatttgaccatttatagaaatatttgtgattattcggatatgtgaccattaagtcgtttccaggtaaatttttcattttgaaggttatcttgatctcttattcccccaccgtgctttctgtcttttgggcattttactaattgcacatgcccgcttgagatgtggagaggggttgatgagtaactgtgggctgcgccagctcagtgtctttaggttttgggttggttgactccatgcgtgcttccctcttccatcatcccaggggtgttccacccttcagggacgccagattcattttgaagaatccttttctagagttgaggaggctgcattgcctttgattcattcttgatcatttttctatgatatggatgagcatttaccaaagtgtgtcgtgcagaagaatagccacttccgaaaggatttgggacttgaataaatttgctaaatgcctacccaccagacatgcatggtcaagtctctccaatctatccacgagaaattcagacgaagattataaatgtgctgcttaaacccctttaggattcctcccaaattgtagctgtttttactttacttaacagtatcaatttttgtctactagtgttgatgtctgggtttggacaaaagttcttagagttggcagtttttacggttgaggttctatactctgaatttaaaaccattcctctctttatttgtaccaactgacttttgcttttttaaaagatagataattcaaagtactgattgtccttgtatgagaagaggaaacctcctcattttggtttatcatgctctggtaaactatccctatcatgctctggtaaactatcccttctccctgatacacacttaatcctctcaattgaaaatatttagaagctctttgtaaaagtaatgaatttgatttggtctcctaaataataaactgcaaaataacatttttttgagacaaggtcttactgtgtcacccgggttagagtgcagtggtgcaatcttggctcactgcaacctcagcctccggtgctcaagggatcctcctgcctcatcctcctgagtagctgggactatggcacacaccatgcctggctgatttttttttctttaagagatggtttctcactatgttgccccagcttgtcttgaactcctaggctcaagtgatcctcccacctccatctcccaaaatgttgggattacaggcatgaaccagtccgtaccctgcctaaaacaacttttttttttttaacattagcagtgattgataattttgttaacatttagtatatgtaaaaacatgcatatactcctgaatctttttatcatagaatttatagattgtttaggaacattatttctgataccctaatgagtacttcatcccttcctccctttgtgctggattgaatgctgtggtgggtacctcagtgtttatagtagaactactatgctgtccctattttcccaggatgggtaacacaagctgcaggtgtaacactgagactttcactttgacctatgaacatacacttaatttcattattcttcagtttttggttattgttaaattagagctcttggttacagggttgaccaccatttgtagaagttcatttgccaagagtgactttatagtataaagtgccttctggctggctgggcataactgaggttcatgctttatcaagtgaaaatggaaagattagtggagatttcaagtcatccattagccaaggactgggcatgccatgtactcatgctgccaccttgtggaggttaatgaaatagaatattaaaaccaagggacagaatgaaaatttacaaatggttgaaaagctcccatactgaatattatattaagaatattgccgggcgcggtggctcacgcctgtaatcccagcactttgggaggccgaggcgggcggatcacgaggtcaggagatcgagaccacggtgaaaccccgtctctactaaaaatacaaaaaattagctgggcgcagtggcaggcgcctgtagtcccagctactcgggaggctgaggcaggagaatggcgtgaacccggaaggcggagcttgcagtgagcggagatcgcgccacagcactcccgcctgggcgacagaacgagactccgtctcaaaaaaaaaaaaaaaaaaaaaaaaaaagaatattgttttaaatgagaacacatggacacagggaggggaacattacacatacggggcttgtcggtgggtggggggcaaggggagggagagcattaggacaaatacataatgcatgcagggcttaaaacctagatgacaggttgacaggtgcagcaaaccaccatggcacatgtatacctatgtaacaaacctgcacgttctgcgcatgtatcctagaacttaaagtaaaaaatatatatatattgttttagtaagttagcatgagttttcatttcaagaagcttgtcttgtcatttttactatctaaattttgtggaagtctggagctgtgttcacatgaaaatctggtaaaagggaatgggccttccttaagtactgaagactccaaactgtgtaaaaccccaccaaaaaaatatgattgtatgaaaaggactctaggatacaatatcttaaaataggaaggagaaagatttgggttatgttcatgtatcaatgactgttgtattgttttcttctctttttgaagtgatttgtgtgactttattgaaatgggcacattttataaactttaatttgtaggcatcagagaccccaactctacaaggcctttcctttactgtcagacctggcgaattgttagctgtggtcggccccgtgggagcagggaaggtgagttagaatggcttttggcagcttgttgcaaccccacttatcatattaacttcgtgggtttgagcccaaggctacgtgtaacacaattttgaactgggtacacttggcacggcatttctcagtgcatggaacattcgttttgcaagaagggtcacatgaccaaataaatgtggacaatgcatatcatgtttcccttggttcttcaagttgagcaattgggaactcctagaagttggacatctccatgaaagaaattatttatgtgaccactatgcaggatggattgactaagaaagtgggaacttctttttgtcagaggggaccctagatgctacaagacatctggaggttatgaaggcttgtctgaaaagctttaaacatgttttgatatgacttctccaagtcacatgaacacacatttaataaacctgtagccaggtgtatagtcctgtgttaggagtaaaagatacacctgtaatcaagacccagacgccactgccctccagaagtttctggtatctggggtgagacagatatacaaacaggtgatttcagttcagtgtcgtaagtgctctgatagtggggggcacagacacgtcatgtctgtgcattgtagaagatgtgaaggaaacgtgcgtacagacttagtctcatatattctaaagcaacgcaataaatctagttgtaggtgtataacaaatagggtggtttaacaattgaagtttgctcttcagagacacgattcttgggtatttaaaaataaaagtccttcctagtgcacaggatttattatttatttttatttattcattcatttattcacgtatgtatgtatatttagtttttgagacatggtctcgctctgtcacccaggctggagtgcagtggcgtgatctagagtcactgcaacctccatttccctaggttcaagtgattctcatgcctcagcctcccaagtagctgggattacaggcgtgccaccatgcccggctaatgtttgtagttttagtagagatggggtttcaccatgttggccaggctggtctcgaactcctggcctcaagtcatccacccgcctcggcctcccaaagtgctgggattacaggcgtgagccactgcacctggccaggatatgttatttacatacagcatttctaggattagttctttgtcaaaaaagatacccgcatgcaggaacaatgcattcagtattggatatatgaactatatatgtcatgcttggctactctgttgctgtaaacattcttagttgatctttgttccgtgtaccatttcatttaggggcatgaaactcatttagggaaactcattagggcagagtagattgttacactctattttgttaatattccttataaactactagtttatgtggaccattatgttatttccaatttttaatcacaacttcctgtgctcttttaaatgaatgaatggtgtttttcaagctcccagggctgggcaaggctgcattgcagtggcttattcttgtctcgagtgttgactgaacatgtgcttttttacaatctgtttcagtcatcactgttaagtgccgtgctcggggaattggccccaagtcacgggctggtcagcgtgcatggaagaattgcctatgtgtctcagcagccctgggtgttctcgggaactctgaggagtaatattttatttgggaagaaatacgaaaaggaacgatatgaaaaagtcataaaggcttgtgctctgaaaaaggtgagtgatgacttttggatttcatgtactcgaatgtccagtaatactagtgttcacttgaacaacaaggtttattaacttttttttaatttttaaagacctttgaatgttatttaatactttaaggcggtcctcccagtaaataaacacacacacgctctgtccttttacgtctgatgcgaaaatgatgtttacagggtgaatttcgggggtttggttggttttgtgaagttttatttacttttaacttttctttttttcaaaatggacaattataaaaatttgcaaaagtggagagaagggtgtcttgaacctgcctgtacccattgctcaagttcaagtgcgatcaacttgtagccaattcaactccgtctctcccctggccctctgctagctccacccccaacccagattcttctgatgcatacccttggcctcaattgtattttatctgtaattgtattttatctgtaataccttattacttatctctagaagataagaacacttttgaaaaaaaattacatctcactcaagaaaaaaaatcaccagaatttcttgctatcatcaaccggctaggcagagattacagtgctttagtatctcattacccattggtttgcgtcagactccggaagatgtctgcaccgtgcacttggctgatgtttcctgcttgggtccctctcaaaccacaggtttccttctccactccctttaaaaaagaaaatttgtttcagtagattttggggtacgggtggtttttggttacatggataaatttacagtgcaatttattatttaaaggaaactgttttaaaatttatttttcattgtgtatcatttcaccaagagagtgattgttttcagaaaagatgaagtgtttgattgtacactgaggttttcataaacctggtggatagttgatgaaaactgagcatacttaatagagggctataagttctagaagatagaaaaaatggtaaatactgataaatactatgttaccttctttgggccataaaaatcagtgaataatggcattaaagggctctccccaggccctgtgcagtgtggcctggacatcctactgttcttttatgctgtctggaaaaggccgccacagactgtcacaggtgtgatgcagaaggcttccagccaggtttgtgaggaatgagtaattttgcttgaacaatggctggaatatcagagtctgtcaatcaggtgagtagtatttgctggcgatttggatagagaaatcattgcttgtttaatgggctgtttcttcatcctggactattgttaatgagagatgatgcattttcggaaatgacattaagttagcagtaatgagctagatttttattgaatagtatacactatataggaaaagatgcgctaaggcaagctcctagttattcagatcctaccaggtcttatttttttttctctgtctcacacgctcacacatatgcacacacaaagctgtgtaaagttttaatcttttgccatgatttcccagtgcctttgctattgctatcctcattttgtttatttttctgatattgctagcctcgcttttttaaagtaaaattttcattttattcaaagtttgtacagaattgctaacatttccctaaaataattactatacttcagcttgtcctgctctggtgtccccccactgtcccctgcttccctgaagatccattttagagaaattttgcagaaatatagggtcaaagtaggagaatatacaaggtgatttcagtgtgttgcagtgagtggagtgtatcatgttttagggagttgagaaacagatggcaaattttgctagttttcccccactggtagttacagttgataaccaagaaactgctgatggaccaggccccggagataggaaagatggtacgccatttttcaatggaagggtggcacttggcaacctgggccttgtctttcagctcttttcttccatcatttctctatgacaggttagaagtgaggtccttgggaccaacacagggctttcttgggaaggctttgttgctctttacctactaatctgtcagtttggagataacctactagtaaactctagaatttaagaatgatttgttcaagactgtggtttcaagagttgaatgttgcttctggctggtaaagtggagggttggagagataaaatgactcacttgaacatttgcagttcaagttcaaagccatacctgcctagctccagattccattttcccctctgccaggcacctctcagttgggggtgtttataaggtagaaaactattttaacaattaggtgattgataaaaaggtagaatgtgggacaaaatgtttttcaagccttccctttttacctgttcagcttctgatacccacatgagactccaggtcccaggtcacccagctctgccttgctccttcccacatcagaaagtctctgggggaaagtgggggttaggacagtggctgccattcagcctcttccgtagccacctttgaggactggcccagtaactcggatagtgtcccaggcacctgcgggcttctgcgtggcaggcagactccagtggcaatggggtcccctcagggctgggctgcatggcgacttcttggttccttcctggatgtgtgtaagcatggactgctggccccctgtgcatggttccatcctcggtaggacggtgggcctccggtggtggagaagtcgaacagagcaggagtctggcccagctttgtcatttgctcgctgtcactttgggttttttttgagatggagtctcgctctgtcacccaggctggagtctaatggtgtaatctcgcctcattgcaacctccgcctcccaggctccagtgattctcctgcctcagcctcctgagtaactggggtcacaggtgtgcgccaccatgcctggctaatttttttgtacttttagtagagatggggttttgctgtgttgaccagctggtcttgaactcttgacctcaagtgatccacctgcttttgcttcccaaaatgctgggattatttgcatgagccactgcgcacgaccagctcggtgtccactttggacttgtcacattacttgccttgggcttttgatgtctctgctgagaaataaggacatgggatgagatggtttccaagaaattttgctaccattctcaaatgccatttatgactgtttagcaggggactggaagaaaccagggatcctcttaatgagttatgagatcatttgccgactccttttcaaatccacgtgtcaatggtgggtgttagatggccatagagaatagcaatactggaattattctggcttgtaggtacagagattgcatgtgtaatcaaaacctgctggacagaaatggtcctgagcgtctctgggtgtttggcctctgtttcatttcttattaaatgggggagatacagcaggattataaagactgcaggcaatgtctgtaaaatgcaaacatgccaggtttttggtaaatagtagctgttgcccttttctctaaatcagttaataagataaataaaagattttgccttatatagttcctcattcccctcctggccaccctcccccgcaaaaaaaaaaaaaaaagtcctagaggaaaaaataggaactaacaagatttctcagatatgttgatagcaactagatatattgtactagatacagttgaagtaaatttataagttcatcgagctaaaaatatatttgagataaaataataagataggaagtagtaattcacatttagcagtattgctttaggctaattatttattcaaagagatatcgtagcaaatgacaggacatacctcaggaaacaacatactaatgttagatactttagtgggtagtagttaaaatccgaggccttggggtcagacagctctgggattgagtctcagttcctcttcagtaggtgtgtgattttagctaatcgtctgaaccttagaaagcctcagttttgtcatctgtaaaatagagataataaactattcacctcataggctattgtgaggataagatttgatcatgaacttaaagcatctgattactaggcacataacaggccttcttaattataagagccaccaattttgggtaaaatcacatgaaatgtgacatgcttgaaagaatgtatctgagtatataacttccattatttgaattgatatttatatagggtttctatacatagtacagtaaataagagaataataaaaatattgttaccctaatagatgcaatgtattaatattatgtcaccctttgcatagtttcttttaatccttgagcacttttaagcagtgtgtatccaaaaaggcatggaattgataaattggagtttaatgtttttctatatagctcacttgactctacaaacccctttggtgtacccactatcttataatttcttctcatatttgccacattcctgccaacttggacattttctttttagattgccttgacaatcataaaataactgttgttatgcctttttgttgttgttgttaatctctcagttgggagaaccatatcactccttagtgacataataggaaataaatgctgagggcccggcgtggtggctcaaacctgtaatcccagtactttgggtagctgaggtgggaggattgcttgaggccaggagtatgagaccagcctaggcaacatagcaagaccctgtctttttttttaaagctgagtacaaaagggatacaatgaggtgaatctaactagggtgactaaatgaactgatagtgctgtatatggatagagagacttcgagttctgagttgtttcagtattgcctttaagtaacttctagcaagcacttggggaagcatgatatgaaagggcaacaggctgtgaactgagatgagcttggttcagactccagccttcctttattaactgagagtcctcagtccagttacgtgctctcacttcatctcagtctcctcatctgtgaagtgtggtcactacttacttggcaggttgtcatgaaggttagagataacaccaggcatcaggaacagtggttggtgcatagctcttacatccaggagctgacctgtggagcaagaagctgagtacacacataaacattaagaggcagaatatggcaggtccttcctaagaaatataaacaaagccttaggaacctagaagagagagtgatgttttctgattgggggactgcaggttagtttcatagaagagtttgagctgggccctgaaagaagaataggatgttgataagctatgacaggaagggccattccaaatggaagcaaggtgtggcccaaagctggagagccctgctctctgactctggggcctcttcttgtgctggggcctcaggcctggcccctgtgaccatccccatagtgtcctcagtggtctttctgcctttagtgtcaccatggttgatgtgcacatggtaggattctggttagatggcgtcaaacccacatagccacgtcatggtttttctctagtttctgtgtctgcatttcagtaaaggtggtggtagattaaataattggttaattttactgagtggtacctaacgtgcatgagataccttcctcccattgggagcacttctgctttctgctcaactgtttccctttagccaaactagaaaatcatggctaggaaattagcctgactcagattgtacttcagatcctttgaaatattttccctgtctgaggcgtgtccataaatagtttgccaagtcaacaacactcaaggtcgttgtagtactcaaagtgctactaccaggcatggggagagagctccttccctattccctgtctgcatgaaatgagacagatatttttagtgtcttttctagaaaatggctaagattgctctgctacaacttgatgctattttgagaatgaaaacaaaacaatgtaaaatcaaggcacagaaacttaacacattatataaagaccatcagcaatggaatgaggccaagtaaacaatgtgtgtttgtgtttgtgtggaaggctgggaccctgtcttaaaatggtgcattgtctttttatgaaaactgaaaggaagagatagtgttaactggtgcattgtctttttttttttgagatggagtttcactcttgttgcccaggctggagtgcaatggtgcgatctcgctcactgcaacctttgcctcccaggttcaagcgattctcctgccttagcctctcaagtagctgggattacaggcatgcacccccatgcctggctaattttgtatgtttagtagagacggagtttcaccatgttggccaggctggtcttgaactcctgacctcaggtgatccacccgtgtcggcctcccaaattgctgggattacaggcgtgagccaccgtgcctggccgcgttgtctttttatgaaaactgaaaggaagagatagtgtaacttctgagctcaacaaagagataacgtgaaataatgaattttcataatggggcacttgctacctttcatttgatgccatctggttcagattggagtattaggggactcctgtgtgtcttagtgggctgccagggagtgggttccaatggaaatactgtttttttccatttggtgatgtactttatttagtgggattcatgctatgaatggggtcctgctgttggtttgatcagaaaatatgtcatagtcatcaatagcaaatgataatgaactcagatccttttttcacttagagagttcatacccttactttagattgttccggcattctatgagtttattaattcttgtttctccaggatattttgtaaaagagataaaatgagaacttagggaggtcagaggatttgcttgtcatggagatctcaatgtgccatttgctttgtctccccggctttgtcagagtagagacaaagaaatgacaccttgatctgtaagagggctatctctgtagtgtcctaggttgatgcttatgcagctcagtcaatacccagcgatgttctccacaccttttgtctatgctaggatctgtctagtgataattataggccatccaagtaaagatttctaatgtaatgtcctgtaacttaaacctgatcattatcctgttattttcagggatgttcttgtcatgttttttaaaaaaagtattatgctttgtcaacttatttcttaaataatttttaaatataactttggttggatcctcaaataaaaacttgttttccttcctagcttttgttttttggggtcttgcatggagccagagtctacttagtgcaaccagtctgtgcagtgtctcatgagcactctgctcctttccctttggcttttcctgactgggctgctctttctgctcttattctttgcagttttttttctgtcagtctattgtgatgccttaagcatcactgtgaagatggggcgggtgttatcccttctctgagaatggaaagctgaggcaaaagtcgcaaaatggcttgtgaagacacagttgttccttagtaagtcccgtggctttatgctgacagccctcacggttgggtctccagcacagacctccagactcacttatccagccgcaagtcaggatagccggtcagcatttcagtttggccataatcaaccttttgattttatctcttgaatctgcgtccctcatttcccccagctcagtaaatggtaaccctgttcttcccttgtcttacaccaaaaccaatgcagtcctcctctttttctatcacaacacaaacccatctcatcagcagatcctataggtgctacctttaaaatttgttcagcacatgaccactgatgttcacttccttgttccccactccggtcccggctactgttgtttcttgtctcaattattgccttgatcagatcatgtccttcctcctatttccctgggtgtgagcccttggcttctcttgcctctgtctctgccctcccctgccatcatcctgtgctcgcagttccgtgtgcatccatattgctcttctgtgaacaccttgggcacgtatcaccccatgggttttgtgcttgctcattcctctatggagcactcttcctgcactctgtgccagccagtccatcccctgacaggtctctgctcagaccccctcgatcagagcccttgttgacctccctatataaagtggactttccctctgtcactctgtctccctaagccagcttccttctccttcttagcacttatcacctcttacatttgtgtcaatcttttattgtcaggtcactcactgtggaaactctttcgctgctgaattcacaaggagttgctgctcaaaaaatttctgttaaaatggctcagtgcataaatgctatgaaaagctacagcattaaaatagataacgtgccctgactgtgtatttgataggttcaaacttacaatttagttactatgaaatatttccattgctgttgatcaaatacatgtaaatttctgaagaactaatgtgcaaagtttaggataactttggaagaacacaaagaacagaggaagaacatgttttaagtctctttttccccccttctttcctttgtccatacaggatttacagctgttggaggatggtgatctgactgtgataggagatcggggaaccacgctgagtggagggcagaaagcacgggtaaaccttgcaaggtaagttgcatttcagcagccactatgccggtgtcttttgataccttccagtggtggtgagcacataggacctgcttactgtgtattgcgtcctgtgtgaagcaccttcttgggttattctgttggcatttgaatgaacatcccagttgttgaaagttgtcatgttcctgggtgagagtctgatacctctttgtattttctagagcagtgtatcaagatgctgacatctatctcctggacgatcctctcagtgcagtagatgcggaagttagcagacacttgttcgaactgtgagtgtgtttgtgtcttaagtcatttctgcttttccagagccatttcgagttggctcaggaaagcctttatggctttcagctcatcctactctggtgtccgctcccctccccacactgtcccctgcttccctgaagatccattttagagaaattttgcagaaatacaaggtcaaggtaggaaaatacacaaagtgatttcagtgtgttgcagtgagtggagtgtatcatgttttagggagttgagaaacagatggcaaattttgcaagttttcccctactagtaattacagttgataaccaagaaactgctgatggaccaggccccggagataggaaagatggtactccatttttcaatggaagggtggcacttggcaacctgagccttgtctttcagctcttttcctccatcatttctctatgacaggttagaagtgacgtccttgggaccaacatagggctttcttgggaaggctttgttgctctttacatactaatctgtcagtttggagataacctactagtaaaatctagaatttaagaatgatttgctccatagtagactgtggtttcaagaatctaatgttgcttcatggattcttaccagagtgaacacttaaaacccacaggcatgtaggcctggcttgcctttgaaactagttgtctagaatctggcttccctcccaactcttacgtggtgtttctgggattttctttctttggctcttgcttagacactgagccatggaggtagaactactctggaatcttttctgtatattctataaactcatgtgtgccgactgacacttcctacatagtctagactggcctttttgaaagcattgtgaactggttttgccaaaacagcactcacatctgccccacagtcagcttcctgtttttcgccttgttaaagtcagtgagcctttcccactcattgtaacccttctgcgcacactgaggtagatgctcctgcagatgctactgcatgaaacatcttcgcttgcagaggagggcagtcagccacagagaagataagcacccatgctgggctgagtgagagagagacaagttcagatttcattcttgcccataaggaatttgtctctgcatggatcaagactaagtgcatgggaaaagttcaacagaccccaaggcttaggggatttcaatataggacaatctattttattattattttttttttagataggatctcgctctgttgcccaggctggagtgcagtggtgcattctcagctcactgcagcctcgacctcctgggttcaagagattctcctgcctcagcctcccgggtagctgtgactacaggctagcaccaccatgcccagctcattcagtgtttttttgaagagagggggctttgccatgttgtctaggctggtctccctatgttgcccaggctggtctcgaactcctgggctcaagggatccaccccccccccccccccccccctgccctcggcttcctggagtgctgagattacaggtgtgagcccctacacctggcatacgacaatctttttttttaaatggaagaagtgggcagcagagattggaaaggtcactgagggccgggcttggctggaagtagcctgtggtcctgcaggctggtaagggtttgtggtggctgagagtaggattcaattcctttgaaagaaatagtagaagcagaggtgtgcagacgggaaggagtgaggcctgtgggaaggacagcaggcaggcacctgttcctgtggtgggggatagagaaggtaccgtgagagaaagctctggaagtcctgtgttggccagccagggctgccctcacaaaatgccacaggctggggggcttccacagcggcaggttgtttctgcagagttctggagactgcatgtgtgagatcaatgcatctgcaggtgcagctcctcccgagggctttcttggcctgctgatggctgccttctcacccttttctccttgtgtctctgtatccaaatctcctcttcctgaaaagacaccagtcagattagagtagggcccaccctcatggcctcattttagtgtaattacttctctaaaggttctgtgttggccgggcactgtggcccatgcctgtaatcccagcactttgggaagccgaggcaggcagatcacctgaggagttcgagaccagcctggccaacgttgtgaaaccccatctctactaaaaatacaaaaatcagtggtggatggtggcgggtgcctgtaatcccagctactctggaggctgaggcaggagaattgcttgaacccaggaggtggaggttgcagtgagccgagatagcgccattgcactccagcctggatgacgagtgagattctgtctcaaaaataataataataaaaaaaaaattttaaaaaaggttctctgttcaatttctaaggtgctagggtgagggctggaacataggaactttgaggggagcatggttcagtccataacaagtcctggatagcaaagtggatagcgagtcggctgtgtgagagtaactcatctctgggctctggattccaggggtttggacatgaagtcatccactgagcatggggctctacgtgggttcctgaacgtttacccaccagagtgcgtatgcaaaatgtgcctggtatgggatgatgaagtggatgtgtatgatggtgatggttgcacaatggtgtgtatgtacttagcgcgaccaagctatacaattaaaaatggtgtaaatggtaaatgttatgtcttgtctatttttccacaaccaaaaaaaacgtggatggcagcattgaatctttctgaatgcagagtgcaggataagctggcacttggcattgccgcagcagtctgatcagagcctcgtttcctgctccctcctcattgtgccttgatgaattttccatttcgccgagaagggcctctgtggcttgcttgagtcttgcgcgaggccccaccagttctttatcactcaaacaacaattcccggtgggacgctgtttagagtcgccgcggggaaggaggaaggtattttcattgctgagtatcagtgtgcgtatcgcacaagctccctcagattttcatttgaacacaggaggtatttataaagtcctcttgcacctttaaggattaatgactttggatatatgcttgttagaagcaagcttgttaaaattgttgtttgctaatcagaatcacattctttggggaaacttagggcagggagcacgaatgcaatttaggaaatcactcagcagcaactggagaatgttctcaacgagttagaaaattggattgaacaatatgctatgaaagatggcggttgtgttgaagcttagaaatgtgaagtgttttgtattaaaagtgaatgaattacgcttctatttgccattaacagttttacttcaatcatgaatatatatcgagagttgttaagagttgtgttttacaactatatgttgcaaacactaagtggattttatcacatacaaaaagtgagggtcacttagattaaaggatgctaatccaatttgttgcctgctatagatgtggttgtgttctagaaagcctcaaattctcagacccctgcctctttgagtctcctaatcagatctcccacactgttgcctacctatttgtcttagtccattcaggctgctgattcaaaatagcataaactaggtactccatataaacaatagaaaattgtttctacagttctggaggacaagattcaagatcaaggcgccagcagttttggtgcctggagagggctcacgctctggttcatagacagctgtcttgtcactgtggaaggggtacggcagcttccctgggctggccccttctactaataccatttgtgttggcagattctcatgaaataggcctcacctcctaataccattacattggtgattagggttcagtaggaatttggggagggccacaaacattcaggcacagcactagttcaccttccctctttcaggtcctctaggactcccctacttttaagctacagttctaagtcctcgaatggcaaattgccctttgcagtctgccccattcacctttccaacccttgccatgcaagaccagtcctccagccatgcacacttctttggggtgcctggaaggaggtgtgaatttctcacgtctgcctgtctgtaagggtatttttcctgccgttggacaaccagggatagtttgcctgggcaaaagagaaaaatgaaagacacagatcctacctcctgcctttctcttctgctcctcggagggcatggcgctttctctgtcttcccacagccctcaatgagctgctcttgatccacctatgtgttcttccctccagcccccgcactagactgcgaatgcatgaaggtgcctttgcacagcgcatttgtgccccgggaccattgcacctgctgtatctgccaggaatgcttcccccagatagtctcatggctcatacatcacctccttgcagagaggactttcctggccactccctctaaagtacaacctttgcctggctgtctcttcatggtttactttactatccaatagtgtgttttctgtgtttgtgtagtgagtgtccctacacgactcagggtgttgtctgtttgcttgaccagtggatgtatcctcatcacttagagtagtccctggtacttcgtcagtgctcagcaaatgtatcttaactgaatgaatgaaggtagtgactgtatctgtccctctctccctccctccatcccttccttccttttttgacagagttttgccctgtcacccaggctgaagtgcaatggcatgatcacggcccactgcaacttctacctcctgggctcaagtgatcctcccgagtagctgggactacaagtgcacaccaccactcctggctaagtttcgtgtttttttgtagagatgggatctctccatgttgcccagactggcctttaactcctgggttcaaaagatcctcctgccttaacctcccaaagtgctgggattataggtgtgagccactgcaccaactgactatatctttctcacttgtgtcacctccaacattgtatgtgtcagctcttaatgttgtcttttttttttttttttcctttttgaggcagagtctcgctctgttgcccaggctggagtgcagtggcatgatcttggatcactgcaggcctcctgggttccagcaattcttgtgcctcagccagccgagtagctgggattacaggcatgggtgaccacatccagctaatttttgtatttttagtggagatgggatttcaccgtgttgcttgaactcccgacctcaagtgatctgtctgcctcagccttccaaagtgctggaattataggcgtgagcccccgtggctggcctcttaatgtagtctttatgtaggattagagttaaatagctcattgctacatcaatcacttattatttgtttgttttatggcatgactggaagatttttactcttaataatttggaaagtattttttgagatttgcctaagaagcataatttagaaaaaatttcaagccatcaattacaatgttagtgtaattatttctattatctcatctacaatggaagatgttaggaagaaaacattttgaaacctttgagttggctgatactggacatagcacccatcagatttgatcattaacatctccctttagatatgtatataaaaggactaatggcagccaatcttgatataaagggaaacagtttcattaaaagctttagtactaaacctgaagaggggtataagtactaaacctgaagaggtatactagattgaacaacttagttttgaaataagccaaactaagatatgtgaaaggggaaaaaataatccttttggacgtaaaggatgaagtgatagtttacatatctgcagtctggagttgtggttctgatcacaggcactgtctctaaaatacttgcttatgacttctcagtcctgaatttctaaaagcagtggctgcatatgaggaagactggaggacagggctaacacttattgagcatgagttatgttcttggcccttgttacagattaatgcttttcttttctttttcttttttttttttctcctttttttctttttgagacagggtcttactctgtcaaccaggctggagtgcagtggtgtgatgacggctcactgtagcctcacctcctgggctcaagtgattctcccaccttagcctcccttctagctgggactacaggtgtgtgccaccacacccggctaattttttttttttgtagctcccaggagtctacgtttacaatgtaggagaactttgagtgggtaccgtagaaaacaaacaaacgtcagaaaacttgatggttgaatactgaggtgggtcttatctttgagccatatggtttggactgtttggattctgtttttccattttcttattcagactgtttaggactaatgagagaagttttgcttcctggatgagaccaagaatactgtggagagtcacatgtcactagcatttgttttcgtatctagtattatttttgaagaagagtctggaaaatgcactctggcaaaggttaccaagaatctaagcctttttgcggggggtggagggggggcgttgatatgtgcattataaattatcatctaagaaaccgtccaagaaaaatgccgcgtcgttacgtgacctcgatctttctttctcccctgcaatcctacacggcaataaaataattagtgttttagttaagtcaaatttttgaatgtctatgttaaactaagttttaggaaaaaaaattacacttattgcctccttcccagaaaacttttgaggatttcaccacagcaaggctcgtatttgctgctagtggttttgaggacctagccggctttattggcttatatttgtatttagtttgtatttaacctctagatctttgactactgtgttgtctttgactacggtttcaaaatgtatgtttttcaatgtggagcaccccattcatgcattttttcaccctgtgccttttacatttataggtgtatttgtcaaattttgcatgagaagatcacaattttagtgactcatcagttgcagtacctcaaagctgcaagtcagattctgatattgaaagatgtaagtagtttctagaggtctcccgaacttgcaagtacttgggtgtgttgaatccccttcttcttcctccatttcatgttcctttcttctcagagctgggattcatgtttttaagtattaactcaaaagacttgtagaaatgtcattattacatgctaaaccatattaccgtaaagtatgtaaaattgctacctcaaggttactacattgttaaagaaaatgatgctgttttaatttagaactttttgtttacctcttccatgacttaattcaaattgtattatttggaaattacagactctaagacctgtttgtatccctttaattgtcatgcatagtttggctaaagatcctcttctattaatagcagaaatggctgggcacagtgactcaggcctgtaatcccagcactttgggaggccgaggcaggcagatcacctgacgtcaggagttcaagaccagcctgtctaacacggtgaaaccccgtctctactaaaaatacaaaaaattagcccagcatgatggtgcacacttgtagtcccagctactcaggaggctgaggcaggggaatcacttgaacccgggaggcggaggttgtagtgagccgagatcatgccgttgcactccagcctgggctacagagtgagactctgtctcaaaaaaataaaaattaaaaaataataataatagcagcaacacagttggataactgagcaacccctagtcacagcacccactttcctggaatagccagcagttagtgtcactgcacaggaatttttgagtgcctgatatgtgtgagagctggtatagctataagtaagacaaagtccttgccatcgtcgggctgacattcttcctggagatagatgcaaggaaggaaatggaggggtggggttgcctcgctgggacttgaggtacagtgacgtgaaggtgctgaccatgcggtcacctggggagggcatctgaggcaggggactggcagtggggtcctaaagctggaagttacctggtgcatttacaggaacatcagggaggagcagagcaagccagggagagcctggagaaaggtgggctcagagcagttgtcagaggccctggaactaggacagaggctactggacagtccagagcagtggagtgacatggtttgatctgtgattttaagagatcctctttgttgttggctggaggagctacagcagaagtaatttgaagtcttagtggttcacaagacagaaataaggggttggtctagtgtgtcagtttcctagggctgccagaacaaagtgccacacattaggcagcttacaacaacagaaatgtattgtctcactgttctgaggctgcaagtccaaaatcaaggtgtccgcagggccgcgctctctgaaggcttcaggggagaatccttcctcgcctctcccactttctggggttttgctggcactcactgttgatccttggctggcagtgcagcccttcagtctctgcctctgttgtcacatggcattgtcgctgtgtctctctggctttcttcctataaggacatcagtcactggacctagggcccaccctactccagtatgacctcatcttgtcttgattgtatctgcaaaaatccagttttgaaaaaaaaggccacattcacaggtaacaggatttaggactcgaacatatctttttagggggtcatggtcaccattcaacctataagagctagcgtggccggggacatggaggttagtgagaacttctcagcccagcggtggtttcatgcagcactccttggcttgttgcctcttgcctcagtaccctggtccttaacccttctctgttccaggccctggtcaaattcctgattgtgggtctgaaacccgcttagtgtcttcaaatgttttaatgtttctcctcctgcaatttcttaataaaacgatggaaataacaaaaacgagtgttaattatattttataagatctgtgtgcaccgttctccaagggagataaatttcacattattgcaacaaacggggtatataatgatagcaaagctcttgcaagtcttgtgtgtaagcccctccttgctgcctaaagaacagtgaggaatctgtccactgaagtcccggcaagtgaattctcacccttaagaggattatcgaaggcagaactgctgcctcttcagaagtggttccgtgtgctggctcttccccactcccccaggaatagggagcctgggttcctcaaacttgaaaacaagtaatttcttatgcagagggctgagactagtgtggcaaaacctgaatgctggtacattagagtaaagtctgtgggtggtagaataatctttctccaaaacaaccatttggcgaggcgcggtggctcatgcctgtaatcccagcaatttgggaggtcagggtgtcggggggaatcacttgaggtcaggagtttgagaccaacctggccaacatggtgaaaacccatctctaccgaaaatacaaaaaaattggctgggcctggtggcgctgcctgtgatcccagctactcaggagcctgaggcaggagaatcacttgaacctgggaggcgaaggttgcagtgagccgagatcgcgccattgcactccagcctgggcaacagactgagaccctaccacacacacacaaaccatttttccactggtgggcacccaggatgaatgtgtgtccattcttgcttcttgctgtcgacccagccccttccctaacagcagacagagcacggttccccctcggaacccactctgcattgctcctcagtactcggcttctgaggaaaattgccatctcagtaccagtaggagtttccttgtgtgtcttacagatactatgtgaaaagtggtcattttatcaaactaggtgtgtcacttttacctcctgtgtggaagaagacacaatgtatcatgaagcatttttgaattctccagttatcaaactggtccaaagggcccaaggtgtcgttggattttcctcagtggacccaccttttgggggtggggaggattggggtagcagggagggtcctggccaccttcccccacctgacttgccttagccaggagctgctctcctttgatttgttttgtattctgggagtttgcctaatattttgtttgctgctgtttgaggaaatcagttgggtctcatttcatggggtacacaaatagtaaagatatttgtacattattttctatgactttgtaaggagagaaaactgtgcaaggttgtgtcccatggaatgtcgaattttcagttaggaactccctgagtctttggggcaattatttatttatttatttatttatttatttatttatttatttatgagatggagtttcgctcttgtcatccaggctggagtgcagtggcttgatctctgctcactgcaacctccgccttctgggttcaagtgattctcctgcctcagcctcccgagtagctaggattacagatgcccggctaatttttttgtatttttagtagaggtggggtttcatcatgttggccaggctagtctcgaactcctgacctcaggtgatccacctgccttggtcttccaaagtgctgggatttcaggtgtgagccactgcacctggcctgtggggcaattttagagtaaagaccgtgaaggtgcagttcatggggtctatgtccaagtctggttcccacacgcctttttccagcagggcccatggccagtggaggcttgtggagtgcttcctacctgtgcagatatgcctgttcctacctccctatcagtgttaggtttctttcctggaatatcctcctcaatttactcattccttcaattcttattgcataaggtttatcacagttttcatgttctgtaataagactaccaatacggtcaccaacatctgtgtgataataatcgccatttgtaacacactattaccattttaaaaatattcttcaggacctaacacagagtctggctgagaacagaacactcagttgtgtatcaagttgaattgtattagatttgtcaactaggattccggacctaagtataatcataacttaagatcagttactttttctggattagaattagattaggttaacattatcacctgcacatgtatctctctgaaattctttatttttattttagggtaaaatggtgcagaaggggacttacactgagttcctaaaatctggtatagattttggctcccttttaaagaaggataatgaggaaagtgaacaacctccagttccaggaactcccacactaaggaatcgtaccttctcagagtcttcggtttggtctcaacaatcttctagaccctccttgaaagatggtgctctggagagccaagatgtgagttcacctcctgttacaacatggcctggttttgttcactgttaatggccttatggatctttggtctactctgttcaaagcttcatcttttgtcccaaagtggattctgtggtttcagggctctattccatgatattggtagattaaaacggaaccaaggcaaaccaccctggttggagttatcctttgtatgtaggctggggaatgtcaaggaggtgagtagcgggtgtgtatgctggtgcaagtcctaagctgtgtgtgactcacactaatactacaattcttgaatttactgtagtgaaatcctacttagtctggtggcccccgacttctctttgcatctggaggcataatttgaagacccccttagagaaagtcctgctgtgggccacatgaaattagccacttttcctgtatgttggaatgttctgatgctgcaggtggctgatggaggtattttgccctgttgtcagatggtttatgttagttaggccagaagtagcatgtgcaaagacccaacagttcctgacacacagcagcccaacttccttctttacctttcaagaataggagctgcatcttactcaccttggtgttcccagtggccagcatagagctgctcaatacatcttcagagtatgtgaatctcatgccttctaagtcctccgggcagaaatgtaccagagctcacttaaaagcatagatattccttgccacacaaaccctgactatccaaatccaggagactgaaggattcttgcagatattcagattaggccctcacatgccaaattatcaggagtcaatttctgaaactcaggaaccaaatagattaggatcatatgctatcccttcctttccaaactctggttactgagactcactgcttgaccccagttactgtgttgagctcatgttttgaccccctgctcttttatgtttccatcttgagtgatgcagttgagagttccatatctttttgtatatgattttggaacaagtttctgtattgacacttgaatttgggtgtgctaggtaagtgctgaacatcctgatggcatccttccagcaagccacgccacaatgtccaaatgatatttccttatctctagtagaaccttgaaagtgggatggctgggtccaaggattagtgcatatagctgggtgtggtggctcatgcctgcaatcccagcgctttggaagtccgaggtgagaggatcacctgaggtcaggagttccagatcagcctggccaacatggtgaaaacctgtcttcactaaaaaatacaaaaaattagccaggcgtggtagcaggcatctgtaatccctgctactcaggaggctgaggcaggataattgcttgaacctaggaggcagaggttgcagtgagccaagatcgcgccactacactccagcctgggaaacatgagcaaaattctgagtcaaaaaaaaattaaataaataaaaaataagtggatgtatagtttggctagctatttccacacccttctccacaggagatggatcattttggacttctgcctgtttccccacagctttgtcaacagagtgcattgtcatctttgggaacagttttgtatactgttttgtctcttaccaccttttttcctctgcagacagagaatgtcccagttacactatcagaggagaaccgttctgaaggaaaagttggttttcaggcctataagaattacttcagagctggtgctcactggattgtcttcattttccttattctcctaaacactgcagctcaggtaaataatgacatttgttttgcgccctcaaaatgatatttacagtacctatgtttatgctattatgactgcatcttgcagttcacttggggtctgaattgaaatgtgctgtctttggggaacaaagtgagggctgcctactggtgttccatgtccgttacaaagacctgcgaaaaaagggtatctttcaatgattttaaaatggaatatgcattttcttgaacccttgatatctgggagtttgggcttggcacagacctgagctggaagaactatgcgagttctctgagccttttattctttctttctttctttctttctttttttttttttttttgagatggagttttgctctgttgcccaggttggagtgcagtggcgcgatcttggctcacttcaacctctgcctctggggtttaagcaattctcctgtctcagcctccggagtagctgggactacaggcacatgtgccaccatgcccagctaggctaatttttgtatttttagtagagatagggtttcactatgttggccaggctggtctcgaactcctggccttaagttgatccgcctgcctcagcctcccaaagtgctggggtttacaagcatgagccaccacgcccggctttgagggctttctttcggttgtccatgttagcgattgagagttttgagttggtaagtcatgcccaagtttgatttttgtctttgttgtttctcccctcctaggttgcctatgtgcttcaagattggtggctttcatactggtaagttatttctggtctgatgtatgaaatactccaaaatcagtaagaagactcgcttttccatagagttagagggaggtgtttgttgatccttctcttctaatcctcacttcgtttacccgaagaaaaattagagtgcttgctggtggagtgtaaactgcggtggtctccacccatgtctgatccgcggcatcatgttcacagacagtttcgggaactgaaagttgtgatttaaattgaatttttataaggtagatatacacaagactaagtgagctgagaatcattttccagcatgaagcgtttctcccaaattgtggttcatttttaaaaaaatgtatttatctaaatttcatatatagcttaatatttttttctccccatgttccatagggcaaacaaacaaagtatgctaaatgtcactgtaaatggaggaggaaatgtaaccgagaagctagatcttaactggtacttaggaatttattcaggtaaagtttaatcatttggtgtattgtatgagaggctgaggtgcttacaatgagcctgtgtgagtaactagggcttccaggagtcattcagtaatgtcttaacagatcaggagtctcagtcgaatttactctgtgaattaattagaataattattttaaaaatctactagaagagaggttgactgaagactcttaatttgctctgtactggattttgaaagttaaaccaggattgagcattcagaaaatcagccactggaaatatgtggctattgtctattctaataggttgctttagctaaatcttatatcattatacggatgggcagggggattagaaacggatctccaccctgtggttcggatggttccaattctgtctaaataaaccctggcttttattgaacttggatgaaatcctctggcagcaaggcccccaaaataaaagcattattccctcccaggtatgcaaagctcccttcagcactgctcatctcctgggcttcactaattacttaagagggaagctgagcagggaaaaatccagtgtctgggaattaggatgccttctatttcttaccttgagatcacaatgttatcgcgtccccaactgtgtgattaatttaacatttggcttgccagactaatattaacgccacactctgttttcacaagagtgcttagaatgtatgtagcattacatactgtttaaagaaaatcttcatctaaaattttaaatatttccacaggaaatgctcctacaagctttcctcagagatagagtgagttatctttgtaagaacctactcagtgaaatttggtgtttatttcatcgatcgcttgctaagaaatctaaatctgttaggtgtttaccttccaactgatggactatgtggaaactaaaggaagaaggaaatctattatgttctattatgttcgtgtaggatggcaggctaaggataatgcccgttgatggagttaggaatttgagctgctttcagaggttaaataacagtgacatgtcatctgaaagggaaatatttgttatagaagttacccccactgtatttagcattgtgaatgaagagatgttaggaaagctcttttaatttctatgtgaataatggatcctcattcatgtcttaataaaatgtatgatttattttattcatttaaaaatgtcttgtgtctatgtaaaacagtgggggattagttgcttatttagattaattatcttatgttaaaatataatttgtgttttcttgcaattcaattattcaagaggttttccacatgcaacttaatacggtatttttaaattctttaaagctacttgggcattttctacaaggaaaaggtaaatcatttctcctattccttgttaatatcacatgttggtatcaaaacataccaccttttttagattatgggacataatgtaagtaataagatttggaatatggagaagatcaactacacttcatgattatctatcctacttctgagtttttaatagaacttgcaattgaattagagcctagaatgaaattgtattatacacctgatttttcttttctgtgcagtttccattcagtgagagaaaaataaactattttaatccttctttgtcaatgtattttgtaaatacattatacttatgtagaatccagatgctttatggaataaattaagtaacatctttataaactacctggcttgctttagcaactattgaagaaaatgcagagatatgctgtgttctctaagtgatatttctttatttcaggtttaactgtagctaccgttctttttggcatagcaagatctctattggtattctacgtccttgttaactcttcacaaactttgcacaacaaaatgtttgagtcaattctgaaagctccggtattattctttgatagaaatccaataggtaagtctgacaccaagtttctcagtaagtatgtcttgttaacaacacatttaggatctgattacatttccatctttgaaaatggaagggatgctgggaagaaaatcactatgcatgttgattcattttccacaagaaacttcactgatatttttctcctaccagagaaaatctaaatatgcggggggaggggggggcatacaatcttttattccaatttatatgtgtgtggaaaaataaatgaatgttcactttgcagaatgctttaggaatccatttgctgtgaggcaagtgaagcatcctgcattccctgtaaacatgtttaaatgtagttgaatagttgctaaacattggctgtgtttacatagtgttaggttatgccatttttcagagggatctttgtaatcaggaatgctctgacgtgagacaggccgtgcatttctttctttcattgggaaacccgttcatttctggaaacacagggtttttgtacataccctgactgtgaggtttccctgaaagataatatgtggttaattaaaagccaagattgataaaatgtaaactccccttcaagaacagtttctaactctgtggttttgaagtagacttttggttctataatagagactttgttgaacctgccatatagatcttcagtagcttgtttcctaggtatacccaactatacccagtattttccaagtggttgatgcctctgcactatgcgcttcttatctgtcctgcttctgaggctagccttcctgacctgggttcctagggagaattagttgtgcctataagtccccaccctgctcagttgagtggacttgtggggaggcctgagtgacattgtgcaaatgataactgccctcatagaagcagggaggtggcctggagagaacacaggcacagctttggcctggccattgactagctttttgaccaaggccagagcacataggtgctattacacaggcaagtctccaagcagatgctgtagttcaaagtgtgaactcaagctaactacctgggttcaatcctggtgctggttactaattgttatagctgattatagttacattcatattcatgtcctagttacttaatttttctgtgtcacagttttcttatctgtaaaatagggatactattcatacccaccaaattgaaatcttatgagaattaagcatgttaatacattcctagcacatgaaactgggtgtagcaacatggtcagtgcctaacgaatgttagtgacaatgaagccatgtatctgtgaactgaatcaccttcactgtggggtggaaattactcctaccctctctgttttacatgatttttgtgaagattaaagaagactgttctggaaagcatcttgtgtagatttatttaacaaatacttttcagtccaagcactgtgcaacgtgcaaggatttgtaaactgcagagaccacacaaatatcggtctgatttcagctgttattagaaagtcattggaggacctctgcgctattacagctgtaacgttgcgccactttgccagttaatttgaaactacagtcataacatttggagcattaagtgggactgcctttctgttatcacaggcctcggaatgctcatgactaccattaaccagcctctccatgtttgagacctgtgggcttctcctcatgctgatttatttattattcagggatttgggacccagtctaaatcttggaggtgactaatcttacagcagcacctgagtgtttatcttgcttcctcctcctatatctattagcatgactttattccttcaacaaaatagaagcccaaataagtaccccaagagaggtctactttgtccaaaatgtttagaggtcatgaattatttactttttttagtggtagatgttgaaaaaatgtaaaaattaaaagttttttaatggtagaattaaaattttcatttagataaaatttttgcactttttacaatggtgaaggatacacaaaatctgtgaattacgcctcagaattcacattactgattgtatgcacttcaatagttatatatatatatatatatatatatatatatatatatataaaatttatttagaaataaggtctcattctgttgactagactggagtgcagtgatgtgatcacagctcactgtagccttgacctcctgggctcaagcgatcctcctgcctcagccttccaagtagttgggaccacaggcctgtgtcaccatggccagctaatttttaaaatttattttattctattttgaagagatgggatctcactatgttacccgggctggtctcgaactcctgtgctcaagggatcttcctgcctcagtctcctcaagtgctgggattacaggcatgagccactggacccagccggctttataatataaataaaaattcccctctatgatagtgaggcaggcatgctcgatttttcagtactcttaaacatacatgttaaatatgtataagtatctggcatgatgttgtgccccctgaattgcagagtgttctttgagtggggtagaaagactgggaagcatgtctgaatacttagttattaaaagcaagctcttagaagagccccgggggagtttcctatgagctcagcatcctgatattctcccaggttttagaaaacttaggaaacggggaggacctctgctggcatgtagtttccagtaagctcaccttgggatggtgactggcacagaaactccaggtgattcacaccgggggtctggctagacgtccctcatatccccatctccagtgcccttgtgctggaatattctcagtgactctggtctgcagcatagaaatgtcttctccattattttatgtccactcccaccccagtgtgccatccccctcatcatgcttctgcccagatcctcatctgtgtggttaatgaatgggcgacccttgctggtgactcagagaaccagaccttctggaagggtccccctgactgccccactgtggtcccaagttcacttatttggatttagggagaaggggaaggtaaacctggaaggtctgtgtttcttctcagcagcgaagtttctgtttcttttttttttttttttttgagacggagtcttgctctgtcacccaggctggagtgcagtggcgtgatcttggctcactgcaagctctgcctcctgggttcatgccattctcttgcctcagcctccagagtagctgggaccacaggcacccaccaccacgcccggctaattttttgtacttttttagtagagacgggtttcaccgtgttggccaggatggtctcgatctcctgacctcgtgatctgcccgcttccacctcccaaagtggtggggattacaggcgtgagccaccgcgcccggcctgaagtttctgtttctaggaaggccacgcttctccaacatgccagatattaagttcctctccatttcgaacccacctatggttgcatgaaattcatctttgtttatataatacaatacctttttatctactggcctttcgttaacctgataaatcttaagctagcaggataaaatgtgagccaaaccagtgtgctgccttagaatgacaagagccagttctctctctctttctcttttctttcttttcttccttcctttcttttctccttccttccttccttccttcctctttcttcctttcttcctgctctgttgcccagtcgagtgcagtggcatgatctcagctcactgcaaatgtccacctcttgggttcaagcaattctcatgtgtctatctcctgagtagttggtattacaggtgtctgccaccacatccggctaattttttttttttttgtatttttagtagagacagggttttacctcctattggtactgagatagcagttttcccaagaagccaagtactgacgagcaatgtggagctgggtctgaggggcaaccatgctctgtctgctgttgggagcgggctgggcccccagcaagaatggacacacattcatccaaggtgaccaccagtggacaaatggtgactgtttgtgtgtgtgtgtgtgtgtgtgtgtgtgtgtgtgtgtgtgtggtagagtctcactctgtcgcccaggctggagtgcagtggcgtgatctcagctcacagcaacctccggctcccaggttcaagtgattctcctgcctcagcctcccgagtagctgggattacagtgtccaccatcacacctggctaatttttgtatttttagtagagatggggtttcatgatgttggtcaagctgatctcgaactcttgacctcaggtgatccacccatcttggcctcctaaagtgctgggattacaggcgtgagctaccgcgccctgccaagagccggttttcctttctcgtctttttatcctcctctcacccattgctagtttttattctttcctcttttgatgtaggttggtgatgggatcacgtgtgtctgttaagaagccttacattttcagctgccctcggacacctccttcatattttgattatgacctgagttgaatagggcatttgcagatattttctggtactgttcgctgcatctacctcttcctagtcagtttttgtgtgtgtcttagccctgtttccgttctgttccaatcataataaatgggtaacacaaatcctaggtcaagagtaagtcttcagaaattaagagatgatttttattttaaatgtaaatcctgttttcctggcatgggcagaattaattttgcttttgatatccagcacaagagggcagcatgatctgcagtatttcagtgatataatgtaagagcacctcattgcaaaacctggtttttaatttaaagctttcttggaaattctatttttatagcttgggtctttgtatcttgatatgtgttaatatgaaatttaaaaagtaatattgtggcgtattatgtttgaaacatgaaagtctttgtggaagatatttttctgatgttgtcctggaatacctagcatttcaaacagggtttttttggatttcctaattacaccttcttgactagtaactggaataggcagacgctgacatcttgagtggttggatgcattttaattcctgaagcatacattatgatttgcttgtatgatgtttctgttttctttgtaagcatgagtttgcagagaaagatagttacacagttagtttctttagttttcttccacgccaacagagttagcagagacattttattcttatcaggtctctacaatttcactctttgttttggagaaaagatgagcggtttgttgtcctgttttctcatgcctaattatggtgacataacatttagctaagtacactgactctaatgggtgccagataaatgtttacttgctttcaatttgttaatttggctttaagaacccaagagctggtgttggtagaagtgtttgtttactctctcagggaagtaaaagttaatattgggttacagccagtgtgttttacacaactggtttttctccagtgtgtgcgtggggagtatgtgtgtgtatgtatatttatgttcatcgatgccttcaacagtgtaacaaaaaaaccagattcatatcattcttttttctttatagattaaggtaaatgagagctaaaacactagataaaagaaaaaagaaacatattgacttcttcccatgtttcttttttttaatccagtggaagatgcttgtggtcactgaggtatggaatgcgaatgctagtactagattttaagatagacatattctcctatgttccctaatcaaaaatcgagggttgccccagtgattaagattctcagcttttagccaggcgtggttgtagtcccagcagctcgggaggctgagtcaggaggattacttgagtctgggaggtcaagactgcagtgagctgtgattgcatcactgcactccaacctgtctccaaaaaaaaaaaaagaatgattccttcccagcttgactgaatgggaagttaggatgtgcatcctacaggaattgaggggaggagggtggtagtggtttgaactattgttctgtttctgaaacctggaaagggactctggaatccactacaagttgctaaataatttgtggagaactgaaagcaacctccatgatgagctttttatatgatcagaaagactgtcttgcgatactctataaaatctgatgtccatatcccacttccttgccagaaaaaaagagtcatcaataaagaagaaacataattgtagggatacctatttatctgtttattcagttcaaatgaccactggagttggtgctaaatgtgcatttttaacaagtataatatttcagcactgtcaaaattacctctacaataataggcttgttaaattatagctctataagctagggggtaagatggctctgcccagcacacattaatctatgcctgttcacacaagtgattctgctgagcaggtagcatgtttaaacaagaaatactggtcctaaaacctctgatttatggtaggtgttgttttgacacagcaccattcagacaatttacaaggcaaagcttcgtgctaaaatatttcctcagtgtcatcattcgtcaccagtataaatattcagtagtgatgctggaaaattggtaatatgcccattgatgccaaatcactcgctatgtttcctttcaagtgagatctgttgtggatatggttaaaagaaactatgatttttcttccttgagagcagatgagctgtaagaaattgagaataacaaccgtcctgacagctttcatgtactcaatcttgcctatattgcttttaattctggcttaagtcctgcgggctttccctcccacccccttgcctgatgcgtattgtaaagaacacatttatatgtacattagttagtttaagaggctttttgtgttcaaaggaagaagttagtcatcaccagaaaagctttgttgcaaaacaaaaacacacatttcccctttcattttttcttgttaataataacaaatgcaagtgactataaagggaataacacaaaagattgcatcaatcataatgattctcaatagccacataaatattgttttttcagctttgaatgaaacagctaagagtttgaagtaggtgtggtaattaaatcctgtcgggttatcacaaattaaaaataatgtttttttcacataacaatggttatatgtacttaaactcttatcaggggtgatacgaagtcatattttatgggagttgctcctttactgctaatagtatcataaaagcctttgttaggagtttaaatagttacgtagttacatatatttgattgctattgttattttgtcagtaatctgtatgtaagcgattactgtagcacttgtctacagctggtaaatattgcatcattttggcataatttttaatctactagaatgttctgattcatggtttttctccataataaatcatacatggagatactgacaacttagtaacagagaaagcactgtgtttgtccaataaaatgttaaaaagataaaaaactagggaagaatgtgctttgttcaaagtgataatcatgtaatatttcaggaaattataagatgacattttacttaactgtgttaaattgttttcctatagtgtatataatgtgatacctgcatgtatttgtatatgcctgtatctctctctctctgtctctctcgatatatgtaagttctccacttcagtctaatgtccgtggctatggttttaacgcccccccattaactgatttgatatgtgttgtctttatctttatcttcggctaacttggaactagctaacttggaactcagggctttgtagcatatcccaatatctgctgttaatgtgtgcttagtaatttggcattagcctatgggatgtgtgactgataatagtaattcaagtattgtcatttaaaactgtcccccactgttagatcaacattatagttctctgtgaccttattgtcagaaaagaatcaaagtatcttcatgacttagttcttagctgttggataaccatttcaagggaaaatagaacactgtttctggaataacctaatgatattccattagccacttgcagtggccgatttcaacatgaataaatattgataagtatattactgagagcctaatatgtataaagcatagtttttggacactaagtggaagttgttgggatttgctagcttgaaacgctgttctgttttactgtaaggccaactgcttcccctcaaagatttgactttgaagttctcatatgagaactctggttttcatttataattggagttttaatttgagagttttccatttataactggagtttaatttgagttttatatgagaactgattttcacttataattggagttttcttattccattattttattccttccccctttattataaacatgattatttatattgccatgaagacagtggaaaaaaaatcagaatatttacaacgctgaggttgtgacattttgttttccatgaagtttaaaacatttttgcttttatcaagtttttgatagtgtttatgaaaatgatagttacattctaatttaggtaattacgtatttaccacccttgtcttatgttagccttgtttcccccttgtcttccttgccttgttttccccttgtcttcttggagcttatctgttgtttccttgctaaatcccctgagcccaggattggatacactgtatctgattagtaaatatttgttggctggacagaacaagaaatagttccaagagtgcatttgcatctctttaagcaacacccataaagaacaatgggactttacagtgttgaaataaaattagagtgttttttgtcttacagctggtacttatattaaatttaactcttgcttccaattttctttttcaaaagtacagtttgtaatagattactttggtggactttgtttctgatacgtgatggcctgaagagaaaaaccgtacattggttctttggaagctgtgattcatggaactagaccaagttctcctgttgtttaccagtcaaagaaatgaaactgtcaaacacctaataacttttttgggggtttagttgtgtgttgaaggtgacttttaacactctgtgggttaagcgtctggtactgtatagttttgtttcataaacttcatgcccttaagaagtaaagagaggaattaatccatttaaaatcatcagtagacattgaaaatctgttcttgcacagggagttgaggtgttgagatgccccaattctgctaggatcatagtttgtggtaactaagtcggtcacattacttctcttagcctcagggttctaacaggtaaaaagtggctttttcattcatcaagcatttcttggtcttctactcatcgtcagttgtggtcccaaacattgaaacaaaagtacctgttttcaagggacttgcaaacttaaatagttgccgacagtggatgatgatgatgatgatttcttctccttcttctccttctcctccttcttctccttcttctccttctccttctcctcctccttcttctccttctcctcctccttcttctccttctcctcctcctcctccttccttcttcattcttcttccttcttccttctttcctctttgttcttcttcttctttttttttttttttcttgagacagagtcttggtctgttgcctaggctggagtgcagtggtgtgatctcagctcattgcaacctccgccctctgggctcaagcaattctcttgcctcagcctaccgagtagctgggactacaggcatatgccaccacacttggctgattttgtatttttagtagagacagagtttcaccatgttggccagactggtattgaactcctgacctcaagtgatctgccttcagcctcccaaagtgctgggacaggtgtgagccaccacactagaccaacaatggattacttctaaaaaccatcctgtgtcagaatctatgatctcatatcaaaaagatgcatattaaaattaatgcagctgccaaatggaagtctagatacttttctagatacaacacacatgagcatagctccatcagatatgtcttcttcataattcattggccttgattttataaaatttccaaaaatggttcttttggcacttcaaaaccaattgtgttcaaaagaaatcataccagtgggagtagcagaggtgttcaggaaacaaaataaatgattctttatttcagatcataatctctaagcttcaagtggactcacaacaatttctagctagactcttgactgggcctagatgcttttctgagccactgggggaggtctaagtgctacagctcagcttaggaaagtgtgttggagaaaggggagggacttccagtgttgccctttacaagtacctgtacacgctttgggcttttagaagtattgataggagaaagaacataagtaatgatattttccctgtaagtaatgttatcatgaaattcaactatgattcactgagtgttctcattcttggtcaccaggattaatgtttgagaaatttcttcctcgtttaaatagaagctctttcatatctcaaataaagactcgacaagcttcaaagtacattttgaggccctggcagtggggaagggggagggagggggaggaaaaaaacaggacatttttgagaggaagtttattaaaaacaagttctctgttctggaaacattttcctttcatgtcttgtttgcatttgcattttaatagattatatcacataggcaaatgttccagcacctcttgaacacctggaaagcctgtcttagacttgttctggtgtttagaaatctttaggcagacagtgttccaaatacagtcatgctcgccaggcatggtggctcatgtcatataatcccagctacttgggaggctcaggcaggaggatcgcttgaggccaggagtttgaaagcagctcggacaatagcaagaccccatctctaaaaaaaattagctgtgcacagtggtgtgtgcctatagtaccggctcctcgtgagactgaggtgggaggattgcttgagcccaaatgttagaagctgcagtgagctatgattactgtattgcccccccaagcctaggaatcagaatgagaccctgtctctaaaactgtgcagcaacatctgagaaaacagtatatctcccctggttgatcttcagcctgatagtagctaatcagacttctctctgaacttcaggtggaagtttatagaaatgttcttttgcttcagtgactggtctcattcctcactctgattgaagcacataatagaagagttcagagattttgggagttggaggaaaaaatagcatacccagttctttctgtgttccgcacaatgatattggaacagggaatatagttgaagcagatttacccttaaatatattttctccacatacaattccacacgccttgatcagcagcagtgctttgaggcatagttacaatcatggattattttttttcattacctttacatatttaaaagtagagaaggaaggaaatggttgcttttttaaaagtagtcattatttttaatttttttttcctcctgttggtgtcagaatggcctgggcgcataaaatttatttcctttgggggaactataaatgtattcttcttctctttttggagaataaaatggtagtgacatcttacaattatcccatgcgaatataaatcttctctatatactccctattacttagcatgaaaatgaaaagggagtcaagtatagtttaataagttctaccaatgaactgtattgtagacagaagtatttcttattggtgccaagttggaaaagttagtcttgacgtggtcctgtaaatgaaaggaaaatcaacacatacatttcaggatcatgagtgatcagtgtcactagaaaagtgttcttctttcactttaagattaaataaatagttgttgacataaatagccggggtaataaaagggaacacctgtgtaatccagccatcagcagttttatcgtacaatcagataagctgctgtttgttaaggaattgtcaagtctttagaccagatgaatagatttagctcatacgttagagaattttaaaaactgaacagaagaagctttaatttcccttgatgccaaatgttctaaccactctcatctcctttcacacacgagaagctgtaggagtttaaagctcaattgacctagccttcaggtatgtcttcttccattagttattgctgtaccaaagtagctaaatgttctagatgagattagggtttgaaattttatatggcaaaataagtacctcatcatttttggataaacatggggtattttttttctaatacagctcatcttttctctccctttaattctcatgtccactttcatttgatgtcaaatgagagagaactttggttgtcatttaagagcctcttgatcaaaacagttatcttaagaagcattgattgtgtgtgtgtgtgtgtgtgtgtgtgtgtgtgtgtgtgtgtgtgtgttgtgatgggtaggggagagggagagagagactttttagttagcttgtcccaccacaataagaattatatttttggattctcttaataatatgtgaaatttaaaatttgaaaattaatcagcaaataagatgtcctttgacttcacccaccagcaaatggaaggatttttctttattttctaataggggatttaaatcactatatgatttgggatccatggtacccttttggtaaaaagcatgactttagaatcagcctgcccaaatgtgaatctcagctcttccttgctgaattacttaaactctttctgcttcaacttatttgtagtatggcgatgatattaacagtacaatcctttttttgtgtgagtgacaattaagagaatcaatatatgaaaagcatttgatatggtacctaacagctaaaaattgatttttttgactaattgagatgatgaaattttagtagtcttaaaaaatgtacaaattccaaatttgctagttttgatgaaacaataggagtctgggaagggtgtctgtgttttcctggatgcaaagaaaaatagataaatgaccaagactaacaagccacaaaagaaaatcaatagagtattttttttgtttgtttgcatgttttctttttggttgtgttttcaatggggcttagcttcattccagagttaattttaaatttaatttggttttacaatgagagctcaattttgcatgtaagtaccaaacatctggtttactttttgtttgaaaacgatacagctgaagttgagtacagaagtctattgtgtaattaaggaaagagagtttgaaattttgtaaaataagttctatttaaatggaatattggaattattccagagaagtggatatacttcttaggagtaggagaaacttacattatttttaaaaaatgctataactagtccaaatgtataaattctatgtattcacctttttcaaaaagcatgaaaaatgacaaatttcacaagtgaaagttaaagttgacctaaggcagttttagattgttgagccttcaaattttattcatgggatgtttttagagacagtctgtggtttgatcactccagttaattctgggcaatctttctacatttctatcagatggacattctgatattttaaaacttcctcttgcgcaaaatatgtttaaagcaacctgggaagaaaatgagatttctggttagttcttgtaaactataaattatgtttttaagacatttaaaggaattataagccaaacatagataactcctgttactgcactattaccattaataaactaacaattaaaatgccgtaaagtactgactggatataatactagcattgtgatatataaaaaatagaatttggggtggagagacctaaatgaagataggacacctactaattttccacctgcttgccagatagttgtgtcttcaggcagagacacccaactagtccagagtcttaggattcaacctcagtaatgtcagaaaggccctcagctctgaactgcgatgattctctcattctgtgcaactcttttagaaataatttgataagagcttaagaaagtcaaaaataggttgtttgtaagacatcagaggatttttataatagttttgccccacccttgaatgttttagaggtctgttgtaggacagaggttttggtttctggcagttgcattctagttctctaccaaattgaggatcagccagaggtacaatacatttggaatcttgccctgtgtcttataattttatagcaacattataaatttatttgacaacttaagcttataaaacttctgtcacttccgttgtcttgtttcatctgcagtagcaacaaagcaacaagaaatctgttgcagtctctctgcctctgagcatccacacagtagatccaccctcttctttgccttcttgcccaatactcttgttattgtccctatcggtttttatgtggcaaccgcagggatgtgatgcctgtaattgaaaattaaatgattgccataaatcattaatcagactgcagattataacagcatttattaagcatcttggtaggccaggagtgtgctgggaattttgcataaggtaaccggttactgtcttgtgaattctgtctgggtattttcctgtgtttgtttcttccaacttttatttttggttcagacggtacatatgcaggtttgttacatgagtagattacatgtggtggagggtttggtatacagattattttgtcacccaggtaatgagcaaagtacctgataggtaattttttgatccttgccctcctcctaccataaagtaggtcccagaatctgttgttgttcccttctttttttttttttttttgagagagtcttgctctgtcactcaggctggagtgcagtggcgcaatcttggctcactgcaacttccgcctctggggtgtaagcaattatcctgcctcagtctcctgagtagttggaattacaagcgcatatcaccatgcccagctggttttgtatttttagtagatgtggggtttcaccatattggctaagctggtcttgaactcctgacctcaggtgatcctcctgccttggcctcccaaagtgctgggattacagccgtgagccactgtgcccggcctgttgtttccttctcagagtccacatgtactaaatgtttagctcctgtttgtaagtgaaaacatgtgatatttggttttctgtttcttgttaatttgcttgggataatggcttccagctccatccatgttgctgcaaagggcataagtttgttcttttctatggctgcgtagtattccatggtgtatatgtaccacattttctttatccaggccaccattgatgggcacctaagttgactccttgtgtttgctattgtgaatagtgctatgataaacatacaagtgcatgtgtctctttttttttttttttttggagatggggtctcctctgttgtccagtgcagtggcaagatctcagctcactgtaacctccacctcccggagtcaagtgattctcccattttagctttccaagtatctgggactacaggcatgtggtacctaatttttttttcttttttttttttttaggtagagatggggttttaccatgttacccagacaggctggttttgaactcctgggctcaagtgatctgcctgcctcagcctcccaaagtgttgtgattacaggcataagtcaccgtgtccagccatgtgtgtctttgtggtagaatgatttattttcctttgggtctatacccagtagtggattgctaggttggatggtagttttcttttaagttctttgagaagtctccagactgcttcccacaatggctgaatttccatttcctccaacagtatatatgtgttccttttctctgcaacctcaccagcatctgttattttttgactttttaataatagctcttctgactggtgtgagatgatatctcattgtggttttgatttgcatttctctgatgtttagtgatgttgagctttttttcatatgcttggtgactgtttttgtcctttgcccattttttttactggagttgtttgttttttgcttgtaaacttaagttccttatagattctgttctttttgcttaggattgctttggctctttggacgctattttttgattccatatgaatttcagtatagtttttctagttctgtgaaaaatggctttggtaggttgataggaagagcattgaatctgtaaattgctctgggcagtatggccattttgacaatattcctatccatgagcatggaatatatttccatttgtttgtgtagtctctgattttttttcagccatgttttctaattcttgatgtagagatcttttacgtctttggttacctgtgttcctaaatattttatgtttttgtggccgtggtattcttgatttggctctcagcttggatgttagtggtgtatagaaatgctactgattttatgcattgatttttgtatcccgtaactttgctgaagttgtttatcagatctgggagctgttgggcagagaatatggggttttccaggtatagaatcatgtcgtctacgaagagaggtagtttgacttgctttttcctatttggttgccttttctttctttttcttgcttgattgttctggctaggacttccagtattgaataggtgtagtgagagtaggcctccttgtcttactggttatcaaggagaatgcttccagcctttgtccctgggcattttctaaagttatgtcgcccattcctatatagtataatattttatgttatcatcatgcttcaccccctcagataagggttaagttacgtttaatatagtacttcctttccaatgaatgtggatatcttattaatcatatgtttaaaaatttgacaaatttgtgtgacctggaaaaatccatcacaaataacaggtgtcacagtgtgtatctaatggatgcgtttatctgtctgaatgtgtatgtgtgtgtgtaatgttgatataagaattccaatttgagttacatgtgtgaaactaagaaatattttaacctctgtcttcttcaccttctgaaatgtttactcagctgttacataaacttgcttttatcaattttcagaaggatttagcatttgaaaatgagttagtcccacaatgggagaaaaaagtttttactatgtagtaaatgttactgcttctgcaaactagatgatcacttttgcctgttatcatgggcatacgttttctagacttctagcagtacattggcatgactttctaaatcaagtttttataattttttaaccggtcatatgttaggagagtgctagctctgaaatttgtagtgtttggtgtcataggagatactgtggaatggtgctaccatagctattccttggcatttgtgtcctactgggagtggaaataattctgtctttggtatgaacattttttccttttttagctctccaaagtgataatggagaactttttatttatattttaataaaaaggagagaaagagggaaaggagacagagaataaactctcaaatccttccaaattagaagtcctgttaactatttcaagatgtctttggtcattctcataaattttagatttaattataaatgtatctagaacatttcatgtaggctattttggaaatgcagttgaacttttaataggactcatcattgttagactttgaatgacagaataacatggtcagaaggggcatctcaggaggttcctgttagaagaaccacagccaagtgcctttcaagcttgtggaaatcccatccagctgttctctaggcctgaaaaaatgtgggaaatcgaaagattggtgaattctcttggtggtctactcagaagaaaccatattagtaatgttgatggttcagtatcaaacctggcttgggctcagagcttgtgagcctagagtcacacatgtaccaatccaggtgagaccccagctgaaaaatttctttgccttgttttatttatatagctaggctttttagtacatggcccactttttagctacttgccctcctccacccataaatcatggtgatgagaaagacagagattcttctatcatccattttattccttgtttcttcttcttattattatatttttttggcagtaatttaagagatttgctttctttcttgtttgctgtagaagtcaaacagtatttggaaatttgcggaaaatagcgtttcataaatcttctaatttagtcttcacaaggcttgatattttatagtgagacactgatttttactcatacccttttcacaagtagtatgatgataagatctaatcctaattctagccatctatttgttctttgttctaaaacttaagtgtgtaccttagatatcactatttcttttttttctggtttactcatgatcttttgttaaatttaaaaatttttttttttatttccataggctattggggagcaggtggtatttggttacatgagtaagttctttagtggtgatttgtgagattttggtgcacccttcacccaagcaggatacactgcaccatatttgtagtcttttatccttcaccccctttccaacctttcccctcaagtccttaaagtccattgtgtcattcttatgcctttgcatccccatagcttagctcctacttatgagttgagaacatacgatgtttggttttccattcttgagttacttcacttataataatagtctccagtctcatccaggtcactgcaaatgccattaattcattcttttttatggctgagtagtattctgtcgtatctatattccacggtttctttatccacttgttgatgggcatttgggttggttccacatttttgcaattgcaaattgtgctgctataaacaggcacatgcaagtatctttttcgcgtaatgacttcctctgagtagatacccagtagtgggattgctggatcaaatggtagttctacttttagttatgtaaggaatctccacactgtcttccttagtggttgtactagtttacattcccaccagcagagatatcactatttctgttgcttcagggaaaaccagtttccagtttcctttatgtattgatggtcctctgaaaaagatagagctggacactgtggtgcacacctgtagtcccagccactcaggagactggttcaggatgatcccttgagcccaggagtttgaggccagcctggtcagcatcgtaagaccccgtctcttacgaaagagtcaatacacacaaataggtttactgaataacacagccattttctgttgagggagcaactggctccttggatgattggattagcttctctttttttggcccaaagtagaataactatggtatttttaccatttcccctcagtctcacttctaaggttcttttcaaaaatgggctaagatatgttaaggtttattaaaaacttcattgataccccccaccccccaccttgtgtaagctgcaacttagtcagagagattacatgtggagctttatatttatggtacattctttgttctctgcttttatactgcacctcagaggattcttaaattctttgacttttgaagcttttcgttttctttgctgagtttgaggacaaattttgggcaaaaacacccggatgataaataatgcatctagacaaaaagagaaacaaagtgaaactcagtgaaaagtaaatcgacctaaacagtctaagaacatttaacgttttgattacatctcatgacatttgatatcttgaccccttcagcctgtgctggctgttgcctttgcctccgacttgtaatgtcagaggattaaagctcttttactcctttctggtgaaaatatttgcaatttcagatttgtaactagtagatctgttcagctgtaaggaaacctttcagtgatgagcaacacgacttttcatctgagggatgcagagtctatgactgcccgagagattgggatcctgtagtggttttatttcattatgagcctccaccagtattatttaatgcaaccaggttttgaatgctggtgagaacacagttaaggtcatggtctgcaaggactttgatgtcgtgcagaagcctgattttcccataataggcatgaaaagcttgtgcaatatagctcttaggagatgaatatttgttcctgtaggttacaatagcagacataacctgtgagtgatggaaaatgtcgggcagaatatggatttggaggaaaaaggggaagaactagtatgaaaaaattagccatagaaatcgatagatactaggccaagaatataactgcaaacgtgtcagctgggttttcccaagaatgattgacagacactattgcagatcctgcaagtaaagaatttctgatgttttctttttttttcctttaaagttcaactaaatgatttccttatagatttgaaactcttttctccttatacagaaatttctcctctcttcttagttcccagatttttgtgctaagagaatgcataaaaggcaattattctgttgatatctttttaactatctgtaaagatatccatattgattcttagggccactaccctggtacagtttttggtggcttgaattaataggatggtttgctgtccattttatttgtgtctggagaaaactttaggttttaatgcacttgcaaattccttgggcaatctgtagcctgtttggctgtctttgttgcctttcctttagatgctatgccgagtccatgaagtgctcttctctctaaggaatacagagactggggatagtccccagagttgaggaaacgtagagccccgtaggtgtaccagttatggtgggtggtgggagtgccctgggctagcgctgaaacttcaggttttcagtggttgacatcatgagtgaggagacttttggaagaagcagaactgaaggtaagactgagtaagtggatctgtccaagagtaaggagagatgaggcctggagatggtcaaggcaacacacagaatctgactttggcagtggatcctagaccagatcagaaccagaactagagatcaggaggtcagcatggaatcagtgggaagccttccatctgcatggttgccagcttcgtgccaagggatgttctggcaattatggaagcacatattggtacaggtttgactaaccacccagacttttagcactggagtttgttgttgcttttttaaattttttcccccaggacagctgtcaatttggggagatatacaggtatgaattgcatgggaaagaaccattgaatatgactctgggatggttaaatatcttgaagctgcaaagaaggaacatagaacaaagaacaagatgaattctcatcttatgatttaaactgcatttaaacaatgtccgtagatcctgccttcctggaggtagaggccaagtagcaccatatacatatgcgtgctatttctgagcactcacggccccattgggcatgggctgtgtaggtgccaaggctggaggggctcctggggctgtgggtgttttgctcaccccctacacagcttgccctcatgcttctctgtaacagggtcaagtttctgggttacagctgccacaggaacaagctggcacattcttcttgttttgaatttgaccactgggtatttatttattttcccacctttgattgacatcaacgtttggtgacctccttcatccctcctttgggcggccaatcctgcccagccacaacactggtctcagttctcataatgatgttcccatcatgtctcttttgtaaacagagcaaatacagaaaatgccagtttctaaactttcacttaatttctacccatccactcagaaatacccactgagtcttatcttgagttaggcactgtgtttggagctaggccaagaggtcgtcaaaattgtggggaagatggatgcgcagtaagcagcagagggctgggcttgggattggctccgtggaaggagaggcccaggggccacgggagtacaggagacaagccaaccagatttgaggtggggcgagccttgaaggatctctcacggacctgtcatggggatgggtggttggggttacagcagccctggaggtaagggaaaggagctgcccagctggcgtggagtgattgcaggccaggtggtgtgagggcctgcccaggccaggttgggccatgcagggcagataaaggattttaaactttatccctagagcaggaaggctttgtcttcagtaaaatggatgcctgataacgtcacctttcggtgcttgccggagctccatttcatcagggaacctattaagagtaaaaatagggactcatttggatttttaaacttttttttttagaatcaggatctcacgatttttcccaggctggtcttgaactcctggcctcaggtgatcctactgcctgggcctcataaagtgctgagattccaggcatgagccatcgtgcctggcctgtttgtttgttttaaacatgaacttattttaatgtgtcacaaattttgtaactatctgacacttagttcacatttcaccatattgtttgtggccattttgattctttgattttcatttaattgcattgagtataatagcagcaaagtctatcttcttactgtttgttcctgttccaagcacctcccaaagaggagtaaaccaacatattttattatctctgtaatccagaaaattattgcataaggacatgtacacatttagtttggaactccagggcaaattagtatatttcctgggtttattttcttataaataatgaccaatatttcataaatattcatttaccactggtcatttaaaaagtggaattgcagtaaagttttaattggtaaagtgtaacgtgcttgtgtgtggaaaggcagcaacagacttggagacttgagcttctaaaagtttcattagtactctaagggctttatcttagggaaagaattggtcctttttctgtaaaaataaaaatgctgttcagtattggttccccccgccccaccgcccactttccccaaagaaaataaaactttgctgcttaatcagggttccagatgtctgtatgtatttatttattcagatatttatagtgttctctgtcaaacagtttgcctccctgggtgcattccagcaatgttaaaaacatacatggcaaagccacagagatcaaagacctgtgtcttgtgaaccctttgcaacatttgggccagtctctaaaatgatttatattaaaacaaccaaataagcactcttcctgtccacatcgtgctttaaggagtgttctggatggggcctgaagaacccgtgcaaattggagttcccccccatctttcgaatgagggacaccaccaggaaagcttcctgtggggctgcatttgacccgcaggtgcctctcccatttcctgtatttccacattttatttttgttttcattatttcttcctttaaaatatgggagttaacgattttacccccccttaaatattctaccatgtagaattttagactgtccattaaaaatatgggtttcattttctatattattttcaaaatatgcagtatctaccatctaaatgggaaacagcaagacttttttgtagtgacaaattaatgccactgtgactaatatctctgaaagggaagtgcctgtagcagctgaggcctgtgagctggagcaggacaaagcatagatgttgtgctctgagtaagaaagtctcaatacacgtggccttcctggtgagagctcttacttgagtgttttaaagaccagtgatctggcttagtccatcattggaaagtgttatgaaagcggccaatagacattttcctacttccactgcttgttgcagatcttctcttctggctgggagctggggtggatccggtaggggacagagcagataggtgtgtgaagtgacaaccttgaaaagaacctggccctatctacctttatgcctaccatctgtgatttgccagagttctctgcagagcaaaccatgtaaagtaaaatgttgcataaacctgctactctatccctggcccaactctgggtgtcaagaagtatgctgtgctttctgcacgaatgtgatgaatgtgggaacctttttgagatagcagcacatgctggcagtggcgagccgtgtgcgcctgggcaggctgggcgttcggcaggcatgttcccaggcacttcctagggacttgttgcatcatcaggtgtttgattctccaccccgtccttccccagcgtgtccaccacccttaggtgagagagtagaaaggagttccactcaaactaggagaattgatgtgtggctgtggaatttcctgtctttccgagttagcatgctatttgtagaggaaccttttggcttccatttgagactggatctcctttgtggaattcatccgtaactttaagagtataacttaggctcctgtcactactagttcatgctgggaataccctcagaaaggctactgtcagtccaaaccaaagttacccagatgcttctctttcttttttcaaagttgagatctggccgggtacggtggctcacgcctgtaatcttagcactttgggaggccaaggtgggtggatcacttgaggtcaggagtttgagaccagcctgggtaacatagtgagacactgtctcttcaaaaagtacaaaaattacccaggcgtggtggtgcatgcctgtagtcccagctacttgggaggctaagcccagaagacggaggttgcagtgagccgagattgcaccactatactcactttttgagacgggagtgaaaacccccgtctcaaaacactaaataaataaaataaaaataaatttgagatctttccccacaatgaaagccttgctgtcccttaaatattaataacattaattactgttctacttaattttatattaaattatatccaaaaaatcatgatttgctaacatttttaaaaactgtgttttaattttgctagatgtagtttaaaacctagattgaaatttaactagagattgaaactggaattctttcagtcctaatgactagctgtggtgtatttttaaaataatatttctatagtttatgagataagtaatagtttcatgttgctatttacaaacatatctcctacaattacatacagtaggtagtttttatgcctaatgatttaaattacataagtgaaaagtcaaactgagaatatttgtcaaaatattttctcttattaatatgggtgtttagaatgtgagcaacatctttaatataggaattatgggccaggtgcagtggcttatgcctataatcccagcacttctggaggcagagacaggtggatcgcttgagctcaggaattcaagaccagcttggtcaacatggcaagaccctgtctctacaaaaaaatacaaaaattagctgggcatgatggtatgtgcttgtagtcccagctacttgggaggctgaggtgggaggatcacttgagcccgagaggtcaaggctacagtgagctgtgattgcacagctatactccagcctggccaacagagcaagaccctctcaaaaaaaaaaaaaaaagaattatggacattattatattttacctgatcgtgaaatgtagagtgttcactgaaaactttagtttcacggaaaactttagttgagtttgcaaaattacaatttttatgcctctgtaactttttctgattttgatatcattattcacttttacgagtctaaattaaatatgagcaaaactagaaaccatttaaaatggtgggattctcaagctcgtgctggtgtctcttgcagatagctaaatggtagatgaaatagtgaatgaattgtgagtctctgatttggtctctcctacccaccatcaaggcttatccagagattttcaaatagcttttgcgtaagggagacaataattaagctaattattttccttactgccttatgaaaatataaattaaaacagatgtcacttgctgtcttccatggcatgaacctctgtggacaggagactctacactagactagtaatttagggtctgtttttccccccaagttcagacttaatcagatcagttgcagataccctgaaagtgaggccccaagctcctgggtgtcatcgctgcttggtgatcctctgggcatttggtactgccctcttgcactcctcttcccacctcccaggacccccacttcccctgagcatcatttccttctctatggctcttgtccttatttatttatttatttaaatttttttgagacagagtcttgctctgtcacccaggctggagtgcagtggtgtgatcttggctcactgcaacctccgcctcttgggttcaagtgattctcctgcctcagcctcccaagtagctgggattacaggtgtgtgccacctcctccggctagcttttgtatttttgtagagatagagttttgccctgttgtccaggctgatctcgaactcctgagctcaactgatctgcctacctcggcctctcttgttttcatttaaatggctcctgggatcacatttccttcactacatattcttagaacacttttctagaatagtctgcagcattttccttctttaaactcaagactcgcccttctccacttcccttgatcccgttatgtatttatctttacttttcattgctagaaacacatcccctggaagtatagagaatgtttgtacgtcactgtcttcttccatttagatcctctatcctctctttttaaaaaataacatatatattttttggcatttccccctcctccttttggccttcggttctcagtgtatacaagctcacagagggctaggatggttactgcctctgaatgaccattattcatgcatctgagcattggtaactgacctaggaacaatcagcttctgatgggctcctcttgaaattgtagcgcatggaaggctttagtgaatatcattgtgttgacttagccatgtcacccagtggggtggcatttcattcatattcatcatttacatcttaacctgaacgtgtttggcttgggcaggaaagtggggatcaagaatattgattgtgtaaaatatttttcagaaaggtcaattgctatggcgttttctaagggtcacccaatgctttgcagagtaaaatgcttacccacatcctcttcattctccttttcttctctgcataaggtggctcttgtgtcccttcagcacccttctctgccaccacaggcaggctcttcctttgccattcctcccctcagatgtcgctgttgttgttcttcgctttgctctccttatcctacaaggagagttctcctttgttggaggtgggaggggactctggtctccagaagtgctgttactgtctcatctctctcaccagttggtgacagaggggctaccacagcaggtacctcctctgtgacccaggcaaattgggtgctgagtgtccacgtcgagctgaatgctctgatggtgtctccaggaatggatgcatgtgatggatagactggatgctgagagaaggatggctggaaagtttttttaaaaaaatgtttttagggactgggtgtggtggctcatacctgtaatcccacctttttgggaggtcagggcaggcagatcaattgaggccaggagtttgagaccagcctggccaacatggtgaaaccccatctatactgaaaatacaaaaattagccaggtgtggtggtgcacgcctgtaatcccagctactcgggaggccgaggtgggagaattgcttgaacccaggaggcggaggttgcagtgagccgagactgtgcccctgcactccagcctgggtgacagagcaagattctgtctccaaaaaaaaaaaaaaaaaaaaaagttttaaaatcatagtaaaatgcacctaacgtaagatttgccatgttaatcattttaagtactctatatagtttggtgacgttaagtacatttgcattattgtgcagctgtcaccatcctccatctcctgaacttatccatcttcccaaactgaaacccaggacccattaaacactaactccccaccaccccttctctccaccccctggcattcacccttctactttctgtctctgagtttgactgttctacatgctggacataactggaatcaaacagtctttttccttttctgagaggcttattttatttcgtgtaatgtccttatggttcatctatgttgtagaatggcccagaatctccttccatcttaaagctgaataatattgcatggtgtgtatatacagtcagccagcctctgtatctgtgggtgctcagattcaaccaactgagaatggaaaatatttggaaaaacaatccaataaaaaattataataccacaacaaaaaaataatgtattacaaatacatagcacttacattgtatttaggcattataagtaatctagggatgatttaaaattaaatgggagggccaggcgcggtggcttacacttgtaatcccagcatttcgggaggctgaggtgggtggatcaacttgaggccaggagttcgagaccagcctgaccaacatggtgaaaccctgtctctactaaaaatacaaaaattagccgggcttggcagtgcatgcctataattccagctactcgagaggctgaggcaggagaatcacttgaacccaggaggcggaagttgcagtgagccgagatcacgccactacactccagtcttggcgacacagtgagactctgtctcaaaaagtaaacaaataaaaaataaaattatattggaggatgtctgtatgttatatgcaaatggacttgagcatttgcagatattggtattggtgggaggtcctggaaccaatcccacacagatagagagggacaaccaactgtaccatctttcgtttatccattcatttgttattgatgtcttgggttgcttctgccctttgactattgtgcatagtgctgctgtgaacatgagtgtgcgcatatctcttttagacccccgtagtctcttctgttgtgtatatatgtagaggtggaattgctggatcatgcggtaattgtgtgtttcataatttggggacctgtcatattttttttttcattgcacctggaccatttttatgatcccccaactatatacaagaattctaattccttcacatccctgccaacacttggtattttctggttttatgatagtagccatgctcctgggtgtaaggtagtgacttcctttattttcttaaatcaatgaatcctacaaggcagctttgagttgttaattgtggggagtgccaggtcgcctgacatctttgggcaactctttttcttttctcttctttttttttgagacagtcttgctctgttgcccaggcaggagtgcagtggtgcgatctcggctcactgtagcctccgcctctggggttcaggcaattcttctgcctcagcctcccgaatagctgggattatagtcgtgcaccaccaagcccagctaattttttatatttttagtagagacggggtttcaccatgttggccaggctgctctcaaattcctgacgtcaggtgatctgcccacctcagcctccaaaagtgttaggattacaggcgtgagccactgcgcctggctgggcaactcttccctgaaaacaaggcgcccatgtgatggagcctgtgcgggtttttgttgtgcgtcccctcttgtcgcctttccttggcatgcttcccctttttttaatcctacacttgaaaattagatcaaacacaaactgtgtttagatttatttacagtttcttaactcctttcctgtttgccttttgacaggcttggcataagaagtctttttcaagtagtaaattaatttaggtggcacgcaccccacctgagttttgttacatgctggcagtttagataaatggatattataatctcagcacatttctgcccaacaccactgcacagatttgcaagagtctacaaaagtgataatattctgtcagtttggagcctagaaaaagaagaagaaggagaaaaaaaagcattttcttgttcgtatttctaggatgcctgtggggaagccggaaatgccatatagcagtttacatggctaaatacacatgaaaaaagataagggacccaaagatgtttctttgtaggcacacgggagaagagtcagttcaaattggtaattgtcagctgtgtttggagatacattctccatcgcaaccaaagatttttctttttcccccttaaactcaccatttccttgaagaatgcagtctatttttaatgactttattgatttgacgattgggttacaatgtactttatttactgcagaaatgtttcgttcctgtcagcacttaatgcgaataaatctactttttggcaccactttgatagactgtaaaactgcttatatttgcaggaaaatttgttttgccacggtatgtttatgtcagactcagtgtgactttttctcccctcaaaatcattttatatcatagtgtagctgacaaataaatacacacagtgcagtaatcattgtgtactgtcagagcagcagaaaacaaaatgtgggttttatgacagggatcaaaaaagagatttaatattcacaattaaagccacttatttaatgctttctgtttttgcttctgtattaaggctttccagtttttggttcatagtttgtatatctcttaaaaatagtaaatatacagtaaaccctacattcactgatattgtctgcatgtgaatttaatatttattatccaagaagacccaataaaagtcgatctttatttcctattgttcaaggtataagcaaagtcaaaatacatttgcaatacgataagttttctgttaagtttctttcttttcttttttttttttttttaagatggagttttgctcttgttgcccaggctggagtgcaatggtgtgatctcggctcactgcaacctccactccccaggttcaagcgattcttctgcctcagcctcccaagtagctgggattattggcgtgtgccactgcactcaaataagttttgtatttttagtggagagagggtttcaccacgttggccaggctggtctccaactcctgacctcaggtgatccacccgccttggcctcgcaaagtgctggaattacaggtgtgagccactgcgcctggcctgttttctgttaagattcttaagtttttcttgaaagcaatgtcatgtaatatatagctttgctctattataataaatttaatttgggaaggaagctgaatcatctttaaaaattttttttcatgaaacatttttcttctacaaatgttttctagtacgtattttttcttaaataagagaaatactatccattttagttatattatacatttttgttttacttatttttcataggatagcaggatataatattggcaaattgaatatcatcaaatagcctacaaccttagtcctgaatcagtactatgccacaagagaatttggaatatccacaacatgtccattatatttgcagtccatctgaaaaaataatacacaaagaccaacaaatggataacttcctgtacaaatgaacacatcagaaatatcattatgtttattgctttagctacctttatgctttaatatgattggcaattatccagtaaacttgtgccaggtaaaaataagcacccatctgggttattcaatttcaatgaacatgtaaaataatattttaataattacgcaatatttgtttttttaaatttacttttgaatgcatttaacaaactggctaggagtcctttaaagagaggtaaatgcaatcgtcaacagcatctgcaagctaaccccaaagaacccattttcagccaacttcgtgatctctcattcagattcaaatctacaatgaagtgccaaaatcatgcttttaaaaaatgacagcattagtgaaactttggaggtgcttatattgctagtgtaggcaaaaacgcacagcaatacagagctaggccgttttccttgattctttcaccataggaagaaatgaacttccagggaggtaatggagtccttctgagaaccatgattaaaagtctttgggaattatcagccaaacttaaaatggatctctaggttttcatggggagttgtgttaaaaagggactttttaatttaaaggaaccaagttgataaggtaggttcagggaaccaaaaatgttgaacagatttttgctggattgacaacttgtacggatggtgggtttttagggtggttaatcgtctgcgtctagggaaatggccacagcttgcctctccttcggggtgaaaggtaggttctgagtctgtgatttttccctgctcagagtgaataaatattgtaagtaggtgtggtcagctgtccagctctttactttttcagttgggaaggaaagagttgcacagctatgatgttcagagccaacaaggacttgcagacgccctgtgcataggagcagctgaacacattcagaatcatgtaattgagcaggctgctaagtaatggagaaaagaatatcatgcacagagatttgatgtctttttatgggagaacaaaagagtttcgattctttcacattgagtttagcctagccattgtgtggcacaggaggtttaatgaatcattgtttaactaggatcctgccaagtatttgaccattaaatagactccaatgtgtatagagttcttctctgacagactcttatttgactgtgattaattggcatcgaattgttatttaattacctccacagaaatctaagaacagtgtgtattctgattaagttatttgatgtaggaacaggcatggggctggagaggtcagctcagccctcaggactccctgctactaccaaggccaacttctgtggctgcccaaagctcacagaagatccctgaggatcaaaaattcagacagaatctggattttacctttttcttagaatataaaaaatgttttggcaactcatagcataaatttgtaaagctttttttttgcatgttgaaatcctttgaagttatttattggactgtgttaaacatgttcagtgacagttattgaggtttccttcagttcacaattgaaacatgttacgcattcccatacatggagaagaggtcagccagcaggatgcatggcacagtaggagtaaggcagaagaaagtcaatgattgcacagtctggtttgggagtcactatccttagagaatggcaccacaagggcatgagatcaccactgaggggatagagttctgagaggtggagcctctagatgcaaggggagaccacctgtgcttagcagatgggcagggtagcaggagtggaaagaggcagatattaaaagaaagaaaatcaggccaggcacggtggctcacacctgtaatcccagcactttgggaggccaaagcgggtggatcacttgaggccaggagttcgagaccagcctggccaacatggagaaacctcgtctctaaaaatacaaaaattagctgggcatggtggcacacacctgccagctacttgagaggctgaggcaggagaatcgcttgaacccaggaggcggaggttgcagtgagccaagatcgggctactgcactccagcctgggcaacagagagagactctgtctcaaaaaataaaataaaataaatttatatccctgaagccagcatacataaatatctcgaaaagacgatgatgaacagcatcagatgctgcaggaaaagtcaagagcaatgaggatgagggaaaagctggctgctgcgtttagaaatgactggtgaacgccatggtgtgaagaggagcaggaatgggactgcagagggttgtggggcgtttggataggtgaacattccccctaagatgaagatgatggtttgactaagaggtaatgtgtatcttaaactgtttataactgacagcttaggtagaattataagaatcatagtaatatttctagttcttaaaattttatctgaatttttaaagaagaaattcaatggttaaatttcccccctagctttttaatggtgtacttaaatatatatatatatttggtgtacttaaatatatataaatatatatatttggtgtacttaaatatatataaatatatatggtgtacttaaatatatataaatatatatatttggtgtacttaaatatatatatattttggaatatatatatatatatatatatatatatatatattccaaaaaaagttcatgcatttttattgaacacaacctagcacacagtttggtgtaacacctgttggcggttcccagatattaaaatgatgcgccggggatggaactgacgatcacatttgggtgttggttgatctgtttccagcacacctttggttgctttacatagggtgggtagtgggagggcccattgtcttcaagttcagtcacgttctcacactgaatgttttgctgtcctatttttgagtattacatatacttgtatgtggattagagatagagccgattttgcacactgactggtaatttccattcaaggtaatctcagtgattgaatttgatagttgcctttgtggaagtggaaattccttgtctaggtgaatgctgtataaatatgcgttgctgccaccatctctgtgggaacacattcttgtagaccgcttggtatgtctaggcccttgtttttacccctccaatagtcattcttttggagatattccatgagcatttagccgcaaacgacagtgacaggatactggagcagtaaaaactatgtcttagggattaaagtggtacattcccagaagaggtcaagagattggtgatttcttactctcttagaatcagaggttcttaatgagagctctgctgtcctgtggttcctggtttaggaatcccctgaaatcatatgcaaactgtacacatgtgcatttccaagcgaaaacactctgtggcattcattggccatgcagtgtttgagagcagtcgtcctagagattctacctgttgctgtccagggatctgtaccttctggatgctacgtgaggctgggccaggttcactcctccctggtgtattcttgagatccatgggtaaaggaaatggcctggtctgtcttcctaacttttttctttttagcaaatgtaaaataatcatcacttgctgtagcaaagcatgcatagctgctatagcaaaatggtttcccgaggcaatttatcaggtgttttcaatggcttcaaggataatcccctacaatacttcctggcaatgagtgattcggctttgccctgggtcacacagcatgttaaggtctgggatgaggtagaactggagcccctggactttcctaattaattctgccttttattaggctgcatgtcctcattcattcattcattcattcattcatttatttacttgttacaagttatggcctcctgtaacttgtgtaatttaatcctgaggtggagttattttaaatgttttactttaattatgattagccaagagagagttcctgaaacccttctaagcaggagtgatagccaaagtatttaccatccagtacagcaggcagtagcctatcagaagaggcctgggctgtcagcggctgctttggccatcgtcatttgtgtcagccctgcctggaaatttattggctaatttttattagtagacatttaatagaacttgcaaatgtgtccagtaaagtagcttgatgtcatggaggtgttgacatgagcacacctttctgaacctccaggagctgggtatgagtcacagcctggatgcctgacagccaggcatcctggaattaatttcttactgtctctgagcctccactgtatcttctgtaaaatgggaacagtatctgtgtcatagttttgaggtgctgtatgtggtttctggcactaatttagtgctctgtggggtggtcttcaaacacagtgttgagttcactcttctaggtttgtgtgcctcatttggtgccttaagaaaatggtctgggttaggatgtacctttatattattattatttttttattttattttattttttttttgagatcaagtctcactctgtcacccagcctggagtacagtggtgtgatttcggctcactgcagcttccgcctcccaggttcaggcaattctgcctcagcctcacgagtagccgggactacaggcacacaccaccatgtctggctttttttttttttttttttttcgtttttagtagagacagggtttcaccatgttggccaggtgggtctcgaattcctggcctcaggtgatccccccacctctgcctcccaaagtgctggaattacaggcatgagccactgtgcccagccttttttttttttttttttttttttaagaaagggggcatttcttgctcccttgcccagtctggagtgcattagcgtgatcatagctcactgcatcatcaaactcctgggctcaaacagtcctctccccttggtttctccaagtgctgggattgcaggcttgagccaccatgcctggcagcagtgggatgacatctccaatcatgagtgaatgaaccaagatgaccctcaggaaaaaatcttgtccagttgcctgctctcagtgaggaaggcaccgggatgagttagtgtgggctctgtctgtgcacacacaggcagaagccgagtcaatctcttggctatcttctgtggactactttctccagccaaagtcaaatgtgtaaaacgtccatctctccaaactgcacttgtgcccctcctaaatgcttcctctttacaaatatggctttgaagagccataagtacaaggaatttttagcagaggccctcaacacctgatccttttccactcccattccacacccaagtcctctcctatcagcacaccttaggtcatgctcttcccatcaactaggcaaatatttgtcatcccgtcaccctttcctgcctctcagtcctgatggatgtcattccttccacctgatcaaagtccacattcttcatcaatacattctcttccctctttccaaattctcaggccctgttcccatgagggctgaccccgcctcaccctggtaacccgcaccagctggtgatgcccccctgctgttgtgccttcctcattgccgcctgccttgggcatggtccgtgctttcttatttctgccacctgcgtgtgtgctcatgtcgggttgtgtcactctacatgctgtgctcacttacatgtatctattaacctgctaccagtgtgatcataagacactcagagagggctgggtgtggtggctcatgcctgtaatcccagcactttggaggctgaggtgggcaggtcacctgaggtcaggagtttgagaccagcccagccaacatggtgaaaccccgtctctactaaaaatacaaaaatttgctgggtgtggcggtgcacgtctgtattcccagctacttgagaggctgagcgggagaattgcttggatctgggaagtggaggttgcggtgagccgagatcgtgctagtgtgcttcagcctgggcaacagagtgagacttcgtcccccgcaaaaaaacaaaaaacactccagagcgacatcaaacaagcatcaattttaagtcatgaaacaagtaaaattttaattccaattagcaggttctgatgagtgattataggtgtcttacctgatgggtgagcagcaggaaaggaatgagtccaaagtaggagattattgttacccagaggaaggaaaacttgaagctggatgtggaaaacagatgatgaggttgccttttgccttgaagccatttgggtttttataagtcacttagtgtagacagtctcacctagcatcttccagttggggaaaaaaatgagaaggatctgattagaaaatggaagaacacatacatacttttgaaactcattttctccaatttgtcatgtcatgtaataaaacctggaaattactttttcaaaaatatgtcataattatatgaaaaggcaagaaacaactacactttgaaaacacaacacagtcctgggactacaggcatgtgcctccatgcccggctaagttttgtatttttggtagagtcggggttagcttccctagctgcggagggtttctgagaaaagcagcacagttatccgtagccggtccagtataaaggcactcgcattagtggcaggttcctgagagctacatttgtcagcaagagtttcatggaggagaaagggcgaaggctgagatcctggtaggtggaggcaagcagggactgaggaccttcatgacaggtggatggaagagagggaaggccccctgatgggaacagggacacacggccaggaggaatggaggggaggttaggaagagacctctcttctacctcttctttcctcactgtttctaaaaacttaatagctttgatttgacagatgaataaataaggatgtaaatgaggttattttaaatgaaaagtaaagacatgctggtaattatgtaaaataaagataagtcaaaaattacttacaagttttcatgctgtatgtttaggcatagatctaaggtagggtttgagaagttaggcgtgggttgttgtgggtcaatttcaaaggattctgtttagtttatttagatacgcgtaagatgttttgtaggataactgagtgtctgagtaagccctgggctgggagatcaggagtacagataagatttgggatgtataagcaaacaggaaatctttgaaatgaaagtgagcaaagttcgagaagagaatcagaaaaaggaaaaaatggtcaacagatttatccttagaaaaaaataagatgaagcaaaggatattgaaggaagttgccctgaagagtggggtttctcattcctcttctctctgccttatggaaatcccagaatggcttggcagagagttcccaccgcattagaaaacactgcatcaccttgagcaagattggcttttgtcttcacatctctaacatgaggagtttggtctacaaagtgctgtgaacatttctgttgcctgcagaaacctttcttcagaggcaatctctatgtgaaggtcccaatggatgtaaggccctatgtttgagaggggaagggcagagaagggaggagatgaggtgtctggggttgggtgtgcttggcctcctcttcatcctctgtctcattctttaagattcttcaaggcagtctcagaatcttcataagacaaggtttgaggctgggcgctgtgactcatgcctgtattcccagcactttgggaggcagaggtgagcagattgcttgagcctcaagagtttgagaccaacctgggcaacatggcaaaacctcatctctacaaaaaaatttaaaaattagccaggcatggtggtgcaccagaggtcccagctacttgggaggctgaggcaggaggatcagttgagcccagggaggtcaaggctccagtgagccttgacctgggcaacagagtgagaccttttctcaaaaaaaaaaaaacaaaaaaaaaccccataaagtttgaatgttatcagccctggggatccttcgcacccatttaagctctataatcctacaataataatacaaagaatgtctcacccatgtgagctactctagaatttagaagatcccttcataatcatgatctctttgatcctcatgatctttggccctgtgaagtagtccaggcaaatatcatcagctccattttataggtcaggaatttgagtgtcagtataggttgggtgagttgccctgtattgttgagtcctgttgctactgagcctccgaggaggataagtacctcatgtcttgtccctgcctcattttctctttcatgccttctgtgagcctctgccatgttgatggcctgttctgatgagatatactgaaatactggacttctagatctcctgtttggttctcattttggtctaagctgagtgatacaggatgtccaggctggcccatcccctcaaatggcttcctcccctcccagatcacagtgtcatagatgctgtcatccttttcagcccagcaactgtctccaggctccacccaacagcctgtactctggaccagtctgtgccccgccatgtggcaccacattgtacctggagtgcacaggtcaattctctcgacaccatttgatagactctgctctttgggacagactgcttgcatttacattctacttctcacatctcaagatagtggttcccccatcctacctttgccctttgcacactccacctaccatcaactatgggtacctgagggtccaccctctctctgtacttctggcttccaattctctgcaggaatatctaaggccaattctttttttttttttttttttttgacggagtctcgttctgtcgcccagcctggagtgcagtagcatccatcatcttggctcatggcaacctccgcctcccgagctcaagcaattctctgcctcagcttcccatgtacctgggattaaaggcgcccaccaccacgcccacctaagtattttgtagttttagtagagacagggtttcaccatcttggccaggccggtcttgaactcctgacctcgtgacccacctgcctcagcctcccaaagtgctgggattataggcatgagccaccgcgcctggctcaaaggccaattcttatcaaaagtattagttttcagaaccatcagtgaagctataagtgtggatacctataaagaagtttacaaaattgaagagttcactaaacacatctacttttaccttcaaggtcagatgtggcacctaagaccatttttgtggtacagaatgggcttaccaggaaggaatgagtggcacatgctacatcatgggtgaacctcaaaatcttgagatacgggaaggaagccagacacaagagatcacatattgtgtgattccttttccatgaaagatactgaaaaagcaaagaggcagaaagtagttgcttgaggctggggtgggcatagggaggcactgtgagtgccatgagggatcttagtgggggcataaaaatgtcttgaaacttatttttggtaatgactgcatcactcagtaaagttactaaaattcattgaattggatacctgaaatggatgagttttatgagacgtaaaatatgtatcaatgcaaatatgcctcaatatcttcatatttaagtgtacttttttgttacaaaaaagaatggggctcacagtagacatgggaacttacaagttttaaattgtagtacggttataagggaattgggaagtcttcgtcatgggagacgtatcagaattccggaggtgactttaacttcagattagattctagaatcagtttgtaaaatagagtcatttatgaatttgtattttgacaaaaagttcattaacaccttttataatattctatgttgtggaaaaaagttaggagcaacctgactctgtatgtgatattatgacttcaaaggagcttggtttctctctctctctctctcagaggaaaattttagtgtgctcataacttaatatagacccttttcagacatttcggttgtttcctgcttctgatttaccaaataatgccacattgaccatctttgatgcacttattaatgccttgagatacttcttataaaattgaattttggggtccgtggggatgcactcaatagttgtttattgtccagctcccaaaaatctacctgtgccatcatgcaggattggagtgatgtggatttacattaaatgtgagtccaaaatgtggtctggctcaaagaatttactgtgattgtgggcttagtcatgggatatatcctgaccctgagatatgcagataatacctagggtatttattttaatcctggctaccaggcattcagagaggctggctacctgactatgtgctgaggcaatggccaacatgggaccctgtgtgttatgagagataattgaggtacccaagggataggtgcctgtgagaagtctggaaaacttgtgattaactttcttcaagtagttgaagagctgccctgtcaagagaaattcgattcactttgtatggttgtaagggcagaatcagcagcaacgattggatgttcccggaaggcaagattcggtttaatgtaagcaagccttgtctaacagaacagtccgaaaatagattgggtctcctcaggaaacagtgactcctcttctctcgacatccctggagatgctccagcaggggccagatgtcatttggatgaagaggtgtggcatgtgcatggagggaatttatcataagtgtttgatttggagacctctgggaaaatgactgtgagactgtgaacttgacatcacagctctgattatgacagtttactgcgaagccagatgaaaatattctggccgggtgctatggctcacacctgtaatcccagcactttgggaggctgaggtgggtggatcacctgagatcagaagcttgagaccagcctgaccaacgtggtgaaaccccgtctctactaaaaatacagaaattagctggtcatggtggcatgtgcctgtaatctcagctacgcgggaggctgaggcaggagaatcacttgaacccaggagacagaggttgcagtgagctgagatctcctccagcctgggcaacagagtgagactccatctcaaaaaaaaaaaaaaattaaaacttttattctttttgcaaacaccagtctttgaattgtttcttagaacaaggaagtaactattgaaccttctaatcttacactttctacactgagatcattgttgataagagttttgatttcagctgatctttgacattcagttcagttattggcgtagctgtgtgaggaggtgggctttctcagttgtttttgttttccttgtattttatgctgttttccaaccatgggtctttcagattttaagatgctcccaaatggtcaagataaagggttggttttcacatactaatgatttatgtagcctttttatctttttttcttcttttcttttttgtagagatgaggtctcgctatgttgctcaggctggtcttgaactcttgagctcaagcaatgctcccacctctgccttcagaagtactgggattataggtgtgagccccatacctggcctatcttttttttttttttaatttgaatattgctttcaactttttaaaagctgaaccatacttgcaatgtttctgaatattgcctatgttaacttttccacttaagaaataattcatattttaataattatgtcttaaattttttaacttatattttccttttattggtattgatacactgagatgagaaaaaataacaagaaggacgacaaacaaaatccataagtatctgtcggaaaaatactcatttaattttaatgtttcttaagtatagctgataaaataatagcctgacctcttgccctgcatagcatggtctggcaaacagcagaagggcccccggaagctggttggaaattcaggctccagctcctccccagggccactgcatcaggacctgcatttgaaaagttgagcttcctccccggtggcatgctgcagaacctgtgggtcatagtcatcatcgcacctttttggttttggttttctagcagccatgaaaaagtacctggcgttgtgcctgggctttttttttttttttttaacaggcatgagcccgtttaatccctaggacaaccctctagaggaggtgttattagaggctcagaaaagttgtcactgggcctgccaaaggtgtcactgaatgatgctttctggagtctgtgtgtccgaagcctgagacaagaaaattgaggtgcagctccaacttgtacacactgtgcttcttggccagctggatgcctcaggagaactgcagaggctgcattagccccagcagcgggaggcagctccgtctacagcctctgaccattcatggtccacttgcagcctggtataaaggccccaggacgaagtcaatcctgacaaaccctaggaggccacacatctcatctgtaagcagggcttccggaagcgttgattagaaaagaagttcagaggcctagacacacacacttctcaggggtcacctgcatccacgtaggctatgtgtgtatagcttccatttttttgaaggaaatcatttgaaagaatagatatttattagaaaccaggtttgatctgtttgtgatgcctttatgacatactgttttcttggattctacttgacgggaaataaagtcgatgctaagcagtgatcttcagtcattaaataatatttctgttttcctgcataatttcctcttgtgttcttagataagactaagcctgtttttgtcatactgtttgcacatctgaaaatgacaacttgtttgccagtagtaaactcacagcctggaaaatgataggatcccaagtttccccaaaaatgtgaggtacagtcaagcaggctggggaaaaaaaaatacagaggttatcattatgtttatataaaaaatataccacttagcaaaagaaattctgataaagcaaaattagtgaaataattaggatcaatttctgtacgttcatctacctactttgcacttggatggaagtcctagattgggtcttaaaaggagggaacattctagaacccaccaacccttcctgatactgtcacaagatttttttcccttaattcttttaaaatcagggccacatttcaactccctgcccgctgccatttgatgatatttttgtttctctttaggtatcgtcctattttctttttctttaccactgacatcggcttatttcatatgaataatatattaaatacatatgcatttaaaatattgttttgatttgtttgcgaattgtttctcttaaagggtctatctggggaaatgagtaatttttcatatcctgttcttgcaggaagaattttaaatcgtttctccaaagacattggacacttggatgatttgctgccgctgacgtttttagatttcatccaggtaacgttgagagtaatgtcaggatctcaaatggaaaacggaagttcctattttttcaagcccttttcatggggtctgggggtgggactctcggcctggctgtgtgtaatgttaacttaataaagggccatgtttgtaaaagaaaggtgcggttataactcagaggctaagttggcatgagtaataccttgtgtgtgggagtggctatacagtcgcgtcagggttagtttgaagcagcttcgtgcttggtaagtggctctccgtctgcctctaggtatctggcgtggattttgctttactgtgaacacatttcataatacagaaacaatcacctctcacggaaagatagcctagattaactaaattatgcattgtattaccacaaagggcaaatacttaggcactttttttttttttttggatagaaccaggtgtggttacttacaaagaaaagattagtaataataaggtgaaaataatactttattaacaaggacaatagagagcagttattgcggatacaaagcctttaaaggcaaggatatctaagccaatttgagtacaggatcaaagtcaactcagaaaatgtcttttttgttttcacacgttgattgctctaagtcttaagctgtctttaaaaaaaaaaaaccaacaatcctacagaaataccttctgtaaaacttttcttttgtttgtgaacttgaggctctaaaaaataatattgggtcatctctcatctccttattcagtactgttgctcatgtggtttggttttattttattttttcctattgtttaattttgtctacttgtcagtggttcaagctggggcactttttttttcccaagtgtttattttggaaatgcatctattaaaagctatacatattccaaaatctcagctgctgttgctaacactttatgaaatttgaacctctgtgactttttctaagtgccaacttagaatagttgtaatcattagctgaatttaaagccattaaatagaaatactacagttggggtctgcaattttggcttgatcctgtgatccccctacccctgcttttttctcaatgctcagatgctttagatctcctctggagccacagttataaaggaagcaaaattacttaatttggaaaattagctatgtaagaaaaatcagtcataggcttgaaatgcttataggaattcctgtttgtcacacagcaaggcagcaaatgagcatgtgactgcaaacagtatagcccatgggcgaaggcatccactgtttatggaggtgaagtaagctgagactcacaggtggattatttttactgcaaacaaggacctctgatttttttttttcttttgcaaacatttttaaaatgtgccctcctgggtttagctatattatttatccctcagaagccatcactaaaacagttgccccgtttacatagaatgcagattattgtctgtacacgttttgacagggattgttaattacttcaagaagattttgtgaaagtttctgtttctttgcaacaggaagctaggcaaaggttgagtttccgaagctggctggcccactatgtgcttagcagagtgagcggtcgtaattagtatcccttatgtgctttgtctcacatatggagggagagggatggatgctacttgatgtcaatataaaacatttcttagaagcatgaggagggaaaattctagtctcgttgtacttctatttaaccagagagacaagggcgtttgttattatccactcacataatcttggtaaacttaatgttgtggaatcctactggccccaattagctgatttcttgaatctacagcgaaggttaaatcctaatgttattgtctccaagaaaggagttttatctaaaggataacttagttatcaattatgataaagatctattgtcacttgttttgtttgcaggtgtctttaataaggcttcccataatcccttcaggaagaaatattttctccttttaaaaatagtattagctaaatataaaagctctggtccctgccattgttgttgattctttgagggacatttcccaacattcattgtttaaatggttgtcaggaagaatcaagtgaaacgtggtaaacaatgcagtaactggttcatcttactttgtgtgccgagcctttctcccggggatttgcattcgatgagtgggcttttgactgaggtatcgccaggatcattttaaagtatttgggcctgttgctatattaggaagtgaaaatttttattaaagccacccacccaaaaaaatgcatttaacctgccctcaaataaaggccaacaaaaatctaaatgtaattttttttttttttttttaagttgcctaggcctctctgtaaggatgaaaacagcccagtgctgttctgggactcttaatggaatcatgtaaacttaaaataaacactattatggtaagcacatagaatgaaaactaattttccaaatcaaatacattatattcaattcattttccccaatcaaccaaatgtttaaaacctgttagttataacagcaatcttctggaaattaattagaagcctattagctctgctagctgagccgaatgaagggggttgctcggtacatttctaagtagtctcctgaattaaagcaaagcaaggtcaaaatgacagttagctttgttcacctcatggctttgcttaaagtcgttgatcttttaggggggaaggagggaggtgttttgttttgcaaaggaaattttgtctactttgactcagttctgtgatacatttttaaaatttatttctagagccagtggggtgagccatacaggaagcctgatgttatctgtgtggatagtagccttctccattgcatgggcatctgaaatggagttgaattttaggcctcgtctaccgcatcaattcttctacagggtttatttagcccctttcaaaattcagatagtaggaaatttcctggatgactttgtgtttaacatccttagcaaaacatgcttcggacactggggccctgggttttgtggctgtcatcaggttctatgggtaataaacatgctgctgacatcacgggccctgggttttgatggctctcgttaggttcaataggtaataatcacagtgactagcagttcctcctaagcccagagaaactcacagaaagtgaggccttctcaacccagcagctgagattgactgaaacaattgagtttaaaaaaactggaaatttctttgtcaagatgttacccttggtgttatctagctgactgatcgacaggggaactacgtatcatcagaaccaactacggatggtattcttgagccagaaagaactttggcaggatgtatagtaacaaatagaatgaagattaaaatccacagaaaagttaggtatcttcataaaaagggagttgaaattctacacttaccagaacattcagactaagactgtcgagttgttattcttactaaagctgagatttaaaatcaaatagctatagaaataaataatgtagttgcatttatccatgtgactttcttttttttttttttgagatggtctcactctgttacccaggctggagtgcaatggtgcgatctcagctcactgcaacctttgccccccaggttcaaacgattttcatgcctcagcctcctgagtagctgtgattacagatgaccaccaccactctaagctaatttttgtatttttagtagagacagggttttgccatgttgctcaggctggtctcgaactcctaacctcaggtgatccacccacctcggcctcccaaagtggtgggattacaggcatgagccactgcacctggcctcatgtgacctttttaatccagagcattcatgtttgattacgttttctgtatgatggtgatcgttttattggttttgtaagtaggagtctggaactgtggaccattgggtcagaactgaactgtttccaattgagtttgccaaagactggaggggtagatctgagaggagaaccctctgcttgtggcttggtgtctagaatgtaggctctgtagaagcaagtctgaaaccatgcacagattttattactctcccgatgaaatcgctgagataaggaaattctggtttacatgtgaccagatttgctggccagatagaggcaattaacctaactttgatttttggacttcaataaaatatttgtatttgtgagttttcatttgtttttatatagttagatgttggcactcattgcagatgggaacagtgacactggtaacttaaaagtgggtgtcatttgctgtcctgtattaagggaagaaacccagaggggcagcgagggtaatgtgtcactttggaactcagattggctcaggtgtgcaacagtagtgaataaagaaaggtgaggtttttgattaggccttttggaaggaaagccagaattaagggaagtttttgaatatatcataagatataaatatatcactggaaataaataacttatagaatacaaatgaacagtctgcaagttacctcaaggttgccatgggctaatcagataatttagccaaggctggcagtgcacatctggcttataaagagttgttcaaacatgtttttcttgtctgctttttcaaaacttgtaacagctttgaacatctacaatatttctaaagccaagtcatttatgaacatagaatggctcttcacaggttaaaagtgaatatcttgttttcataaagaattgaaatacaaatagcataaagatcttcatctggaacttatgttctttgaataggaatttggcattcaaaacatagtatgtttgcgtaaatcaacaaacaaaaacatagagatgatagtcaacattcacattttaaagtgcttgattacttcctttttttctgtggttttttttttttttttctttctttttttttttctgaaacagggtcttgctctgaagcccaggctggagtgcagtggcgcagtctcagctcactgcagcctccacctcccgggctcaagcaattctcatacctcagcctcccgagtagctgggactacaagtgtgcaccaccatggccagctaagttttgtatttttagtagagatggggtttcaccattttgcccaggctggtctctaatttctgaccccaagtgatcctgctgcctcagcctcccaaagtgctgggattacagacatgaggcactgcgctgggccagaagtgcttgattacttcctaatatcagcaccaatgccagcatcatgaatctcttcttccattttcttaaaacaataattggattaataggtaccctacctgtaatatttgtattaataatatcttattagcattttaattagaaatttaatattaatgatttggaatcagtagttttcaacaattttaatagtgcaaatttatttgtagtatttgatggggagatatggtgttttgtgactatatttacaactggatgtatctgggaatatctcaaagtttctcccaaaaggaaaacatttctattacaaatatgtgacttgtgaaaataatttgtaattcttttcaatatatctcattttttttttttttttttgagacagagtcttgctctatcacccaggctggagtgcagtggggcgatcttggctcactgcaacctccgcctcctgggttcaagcaattctcctgcctccgcctcccgagtagctgagattacaggcacccaccaccacgcctgtctaatttttgtatttttagtagagatggggtttcacgatgttggccagactggtctggaactcctgacctcaagtgatctgcccgtctcggcctccaaaagtgctgggattacaggcgtgcgccatcacgcctggccaatacgtgtcattcttattacatgtcttcctttttatttttttttcccaccagctttttattgagctcctactatatgctgagtatgcagaggtggaaaagatagaggctttatcttcatccacctgagaagcatttattgagtatcttgggtgtactaggccccatgttgggcttggagatatggggctaagtgaaattgaattcccttcttctatgagtttactctattgggagggacagatagaaagcagttaccatgctgtgtgataagacttacaatagaagggatactgtaggagagaatggttaaggtctcagcacgagacaaatttccaatcctagctctttcactttaagttcaattatggcttcctccaagactggccatgcaacacctaatgctggagtgaggactcaaaccctcttctcacagtgcagccatatgttagaataaatatctaaatagatttgttttgctttgcttgcctttgatttgtggctacaaaggacatacaaagagcaaaattcaaattttctggtccttaaatctaatctctctttaacttttggaaagaaactatgtgcatgccagactgttggcacctgtgtgtgtgtgtgtgtgtgtgtgtgtgtttgcacacctgtggctcaccctcaaatgtacccttctaattcttcatcctttaaggaccagcgaaagtctctctttcgcattagtgatgcttttctttaccactccattctgtaataaaacaatctctaggtcctgtaaaaagaagggatcatataacaccaggctgagcactgctctgagcatatttcacttgccccatctcatttaaacttcactcatagtcgttgagtcaatcagtatttcttggacacctcctgtatgccaagtacttttccaggatctagagcatggagcagttaactgggtatcagcttctaggaattggcattctagtgcaggaggtcgacaataaacagaccaataaacataaataatctaagttagtataggtctagtgtgttcagcaaaggaaagtgtttgctaatgactggaccagcatagtcaaggaggaggtgacgttgagtttgaaaaataaggaaacagccgtgcaaagaagatatgggggcaggttgagggaggggcacattgtatggaggaggaacaggtgccaggtctccagaagggaacaaagtggcatttcctcaggcagagagatgccaagcatgagcagggtacctgtagtcccagctacctgggaggctgaggcaggagaatcacttgaacttgggttgtagtgagccaaaattgcaccactgcactccagcctggacgacacactctgtctcaaaataaaataaaaaaaaaaaaaagaaaatagatttaacggactcacagttccatgtggctggggaggcctcacatggtggaaggtgaaggaggagcaaaggcatgtcttacatggcggcaggcaagagaacatgttcaagggaactgctgtttataaaatcatcagatctcatgtgacttattcactatcacgagaataccacgggaaaaacccaaccccatgattcagttacctccccctgggtccctcccacaacatgtagggattatgggagctacatacagttcaagatgagatttgggtggggacacagccaaaccatatctatcactaagtctctaataatagattatttgtcatctggtgcccacccatctccctatagaaagtcatataatgctcatttaatatgagcctatgatattttagatataaacctttcagaaattgtggtactagcctttttttcttaccccctgtgtctttagaattaaaggtagacaaacttagtttcctttccccaaaattcttttagacattttgagtggtcagtaagattcccagctgtggagactactcatttaaataactttattcctggttttatgataaacctggtctcaaacccaggtaccagagaaactgtgactgttttttagatatgagcatctggaagctcaggagagagaggacactagattttcttttctttctttcttttttttgagatggggtctcgctctgtcgcccaggctggagtgcagtggtgtgatctctgctcgctgcaaactttgcctcccagcttcaaacaattctcccatctcagactcctccacagtggctgggacaacaggtgtgtactaccacgcctggctaattttattttttggtagatatttggtagatacagggtttcaccatgttggccagactggtctcgaactcctgacctcaggtgatccactcacctcagcctcccaaagtgctgggattacaggagtgagccaccatgcccagcctatatctttatctacagtattctgatgcacctttgttcaggttgtgatatatatagctctgtggtaatactgtataaacaaggaaaaacaattaactgtggcaaaagcaaaggcacatttctatagttactgttaaaaaataactttacattaaaactttgtaacaactggtgattgtacattgtactaatattattcactggaagtgcattctttatagaaagttacataagtatactaaagagttaaatttaaatactataaacatatacagtacacagcataaatatttttgtgaaataaaaacacctcatggtttaattaaatggaatgtttctgtcacaatttatactttgaatactctagttttgttttgttttgtttttttgagaggagtttcactcttgtcaccgaagctggagtgcaatggtgccatctcggctcactgtagcttctgcctccctgttccaagtgattgtcctgcctcagcctcccgggtagctgggattacaggtgactgccactatgcctggctaatttttttatttttagtagagatggggtttcaccatgttggccaggctggtcttgaactcctgacctcaggtggtccacccatatcagcctcccaatgtgctgggattacaggcatgagccaccacgcccagcctgaatactcttaaaaactgtctgtaaaggatctgaactcccccacagccccatgtatttgattttagacaacaaagtctgggtgccttatttcatgtgtgaaagagaaatagaatctcaggtcccagactccctatgccaaagggaaagttaagcttgggaacagagtcaccagtaccatctttcttttgttcccaaacagccatgatttcacaaccctactatcctgtgtcactgcctcatttcctgtactccctcctttatcttaatataaaatacagatcgactgagtgtgagacaatgcacaatccacattttcttctactccctcttttcacatgtaaaatgtagatatactggggctaatcagggtctcacaagaaggtgaccatctcactgtctaccctccctccctttttcttttttttatttttttttcccttctgcttgctcttctttaaatactgaagtttacaaaactccctctagaaaaaagcatcacagatgcccctgccacattctcatctgtggctaaataaacctctaatcgattgagacctgccttagttactttttggttaaaatatacgtgtgtgtgcgtatatatgtatatatatatacgcgcacatatatacgcacacacacgcacatacccctcaagaagcaattaaaccgttgatatgtaatcttccaattaaagtagcttgatgcccatcttaaactgtaaattgaggaaaataacatttgtggctgtaatagatgatttatgtagggctctagggcagtcggagtctacattttagccagccttgtaaacttgtgtatggaattcatagttgaacctatagagagctgaacccctctaaggtctttgagcagctaagtggcagctcatcttgtaattttaaggttaaacaaggatgtgtgatgtgtgcacatgcatctctctcctttagagacgttattcccagttgatgggctttactgaaaatcagcgaggccggtaaccggcttaccctgtgcacacgtggtggttgtcctctgagttgtgcctggttgcttcctgaaaagggtcgttttcagagttctccctgctctttgcggtcacctgaaaaaggtctttgtggcagtggatgtctcctctatcttgctgttatcatcatcatctgcatcagcatcttggggttggacagcagatcactccattccaagctatgctgttgggaggcttcccttggctttggaactcttcacttaccccattgcaattcagtgtccagcagtgccgaggccactaggcttagtcaccatacttctgcctggtggaaacctgattgtgtttcaggtttggctttgttgtatgaggttttctgttttcgggagaattgtggatggagttagtgaaaccaatagcagcactaatggagctttatgcacatttgagtgaaactgaaagttaaggtattaacttctaacttgtccttgaacctccaggggtaaatgttaaatttattctttaattttcttgctaattagtgctctagggaggatgaagaagtgaacaactactcttgttgactaatccagtggtccccaacccctgggccacagaccagtactagtctgtgtggcctgttaggaacctggccacacagcaggaggtgagcggtggacaagaaaacaaagcttgctccccatcactcacgttaccgcctgacctctgcctcctgtcagatcgtcggcggcattagattctcataggagcacgaaccctgtgaacattgtgaaatgtgcatgctagggatctagcttgcatctaccttatgagaatctagtgcctgatcatctgtcattgtctcccatcacccccagatgggaccatctagttgcaggaaaacaagctcagggatcccactgattctacattatggtaagttgtataattatttcattatatattaccacataataatagaaataaagtacacaataaatgtaatgggcttgaatcatcctgaaactacctccatcgccatccatggaaaaattgtcctccgcgaaaccagtccctggtgccaaaaaggttgaggactgtgttctaatccacttgtttgttttcaccaccttctggattcacagcaccctccactccacctgtagcacatgcctgccactgggctgcctctggcggaccccattctctgccccaatccttggcctcattatgcttcctcagagtcagttgtgattcttcctcaacgtattcatgctacttatccttgttactgtaattttgtgatggcaatacttatcatataaactgatgaaacaggaatgccacaagaaagatttgtttgacaagtcgaagccttgggaaagaaatcttcactttaaaaaattgttaacaaattatgtgtggatgagaataaatggggtggagatgagaattggaggaaaataggattgtgtgctaatagcttcacatgtgtcttaaaattcatagatgatgtattatgtgtgtgatatatacataagaaagaccatgaaaatctccaacatcagacccggacatgttttttcatgcctgtaatctcagcactttgggaggctgaggagggcagatcacttgaggtcaagagttccagactagcctgaccaatgtggcgaagccccatctttactaaaaacccaaaaattagctgggagtggtggtgcacacctgtaatcccagctacttgggaggctgaaagaattgcttgaacatgggaggtggaggttgcagtgagtcaagatcataccactgcactccagcctgggcagcagagagagactccatctcaacaacaacaacaaaaaaacccaaaccaaaaaaacctcaaaaccaactttaaggattttgtattgtgtatcccttatttccatgctggccaaatacaagttcacatcacagcagcaaagggctttgtctgttgccctgcttggggaagctggtaaggcttcctggttattcctgagccatgtaggggagaaggaggcacatatggcaggaagtgcaggagtttaggatatctacagggacactgcagtgtggaatagctgcatcaggaggtttgagacaaggcattagaggagatcagatggagagatgcagaggggccagatcacaagaaggcttgtttcttaagcacaggaatttagagtttagagatttgggtggaaggatatggagaggaatgggagactgtcagaaagatggggttgtaccagcatcaggaaaacaagaaaggatactggtttggacaagtttctctttaggacatccaggtcagaatgccttgtgtggctaatgaaagcccttgcttctgaatagggggcccacactgcaggatagtcgatcccctcaggcatgtgtcattcacaaggctgtgtcccagtagaaggccagttaggtttgcacacctccgtgaatgctttactagatcaactgttaaagctgtcagatatcttgccaatcctgtcaaattatagttgcttgcaaaattggagctgttctcaattccaaaattaaaaggaagccagcatcatttatgtggtctgtttagggtgattctacctcaggatatcagagccatgaaaattccctttgcaagtgctcttggttctctaggaaaagcttcaggttttgtttttgttttctgtttttttgtttttgagacggagtctcgctctgtcgcccaggctggagtgcagtggcgtgatctcagctcactgcaagcactgcctcccaggttcatgccattctcctgcctcagcctcccaagtagctgggactacaggtgcatgctaccacgccaggctaatttttgtatttttagtagagacagggtttcaccgtattggcccggctggtctcgaactcctgacctcgtgatccacccacctcagcctcccaaagtgctgggattacaggcgtgagccactgcgcccggcccagcttcaggttttaaaggtgtgagtgttgagtgtgctactgtgaataagattagatgtgactgggccaggtgtggtagttcacacctgtaatcccagcactttgggaggccaaggtgggtggatcacttgaggtcaggagtttgagaccagcctggccaacatggtgaaaccccatctctactaaaaatacaaaaattagctgggcgtggtggcgggcccttgtaataccagctacttggggggctgaggcagggagaattgcttgaatccaggagacagtggttgcagtgaaccaagattgtgccattgcactccagacagggcgagactcgatctaaaaaaaaaaaaggtgtgactagatatcacttgtaagtaaataacttcaaagtataatgtaggggccaggtgtggtggtccatgcctgtaatcccagaactttgggaggccgaagtgagaggattgctcaaggtcaggagttcaaaaccagcctgggcgacatagtgagacccccttctctataaaaataataaaagactttgccagagatggtgtcttatggttgtagtcctagcaatttgggaggccgacctggaaagaactcttgaacccaggagtttgagaccagcctgagcattatggtgagaccctgtctctacaaaaaaataaaacaattagctaggtgtagtggtgcgtgcctgtagtcccagctactctggaggctgagtccggaagatcacttgagcccagaagttcgaggttgcagtgaaccacagttgtgctactgcactccagcctgagcaacagagccagacccagactcaaaagaaccaaaaaataataataaaaaatttcaagtgtagtgtaggatggaatgggagaggcttggaatcatccttcttaagtttgactcctagctctaccgcttaccttctctttcctcacaaagccacatgctacgtctctgtaaaatggagaaaattctacctattccatgagggttattgtgaggataaaatgaagtcatttcaataacattcttagcataacatctaagctcactaaataaaaggtactttttaaatactacgtatatttttaagtgggagaaatagcctgacaatgacattatgctgcattaaggtaattgtcagacagttaccagacccataagtattaggacatgagtttgtattccagatattaggtttattatccctgaaggctttaatggctattttctcttctggaaccacagtatcaaaatcagttgttttcagaataaaccagtgagcactaaatatctcataataacgtaattttcataatattgctgaaacttccttggcccttgataacattgagtgtagagtctgagttatctagacttctgctagctaaaatctttgcctctaagaagtcttcctactagaaaaaaacaaatgaaaaatgaagataagatgcagaggttggatttaggcattttaattttttctgtttaaatttgggagtgctccgagtgtgctaacttgcacacagctgcactcagcaaaatactgtaatatattgaggcacacggaattgcaaaacctttcttccctacacacccctcatctaaaaaaaaaaaaaaatcagcagcttgactgagcttaaataactgacttttcatatcctttcatggaacacaaaggactagtcctgaggctacttgagccaccaatgcaggcagcagtggagaccaccactgaatgtgccagtccctcctcccacaaacaaaacacattgactgcagctttatgaagtgaaatgcccaagaaaaagaaaatgtgaaggaggtgtcctcagtcatttcttccctaggggggtgggagggcaggatgaggggcaggagaaaaagctgctgcatctggatgtggagaacaaagccatcctaagacttgagtataataggttgggtcactcgcttttatagaagcaaaagagtggaactgccttgacttgtctcagtaccactgacccttacatttcattgtgtagagatgtcagggaaagtgtgtagaaagcacactgaacaatgactttgtcattttgagatttctgaggtttgttcaccccttcccccaactcctaccctcaaatccccattggacaaaagagatcgttagttaggtcgccttgccgtcatttgaaagatctctgttttactgagtctttcaaaagtaaactcatccattttaactgaatcacggcgttctcatgctttcaaagtcaacaaaagagttttagtcactgcatttacaaggatggactagtctgtgaaaatgccagccctagagataatgattcatgccaactgccacattaaagtggatttccttgttaatagacagggcagttgatgacatgagttactggaaatatttgccaactcttgacccctggatttctgagatcaggatgataggattcacacaggcccactctaacaattcctttaccttttcttttttaatgaaagatgcaatggttgggttttttccagtgtgatagcacatgtagaaagcagtactgtcaccattagtgtggtgacagttaatcactggggaccctcccaggcttcccaggctggcaaccagtgttccaatttttcccagcatgtgtgttccaatgataatgtttgtagtttaacaagaagctaattaagacctaaagaaaaaaattaagagtctcatttttatcaagtctggtggtattcagcagagcatagaactttccattctggcatattggatgatgtccattgcaaaacaatagtggccattcgtggtgctttctcttcttgtcattttgcatccttagtaatgataaaatggacagctattacccaggagaggaaaatggccaaatgtttacttaattgaggacctttaaattctctctatgctattcttgattgattacattcatttttgtctgtatacctactgagttttcttgataagatgaacaaaaaaaggtttggactggagaacttgcctgagttttattaaaatcttgcagtcactattttctatatggatgtatcacttccatctgatgtggggcatgggtaagtgtaatctgttctacttgtgagttttgtgactcacttgttttctttcagccctttccctgggttcattgacttagtgttctctctcatttgcgttgagtcatgaagccgatagtgccgcttgtgcatcgcagcaaagggccagccacctatgggacacgtatcaaacatggatcaggagccatttttgttctagggcacatgggctggttaccattagttctcacatttgccctttcccaccctcaatgcacctgaggagatcatctcttagcgttatggttgtgtattgatacactgcccaacctctgcctgaacatggccaaagggatgggaatgaaattctagtgtcatattttttttaccctatctatttttattgaggtatagtttatatacaataaaatcgacagttttttttaggttgaattttactaattgtgtacattccaatatccaccctctaaaacaagatgtagagcatttccatctcctcaggaagtctcctttgctcctctgcagtcaattttcacacctctacacctcttccagatttctgtcaacatcaattagttttcctgttcttgaactacgtataaatggaaccaataatttttctgtggctggattcttttgttcaccatgtttttgagttctgtgtatgtttcagtggttcatttctttgttttctttttctgagtgatatttcattgtatgatgacatcaacatttatttgtctactctctgattgatgggtatttgaattgttgccagtttttagcaatgaatgttcttatagaagtgtttatgggtacactacatatgttttcattcatctcagataaaatgcataggagtggaattgctgggttcgtaaagttcactttaaaaaccaaaaatcaaactatgacaaggatttttataatggttgtactattttacacacccagtggcaatatatgagcattctagttgctccacatcctcgccaacatttagtatgatcaatctttttgtttttattctttttagtagagtatagatgatccatttgcattgccttgatgattaatattgagctttttccatacaggctaattggccattcaagtgtcttattttctgaaatactggttcagatctcctgccagctttttaaaacgtgtttttaaaaatcttgttgattttttagtttttaatatacataagtgtatttgtatatgtttaatattaggcatttgtgaatatttcctgttttgcaaattgtggtttttaatacatttgcttatttcgtcttcactggagaactttttgcatgaaactgttcataatctcacatttgtccatttaatgtctttataatctatagtgaaatttcttcttttattcccgacactgataatttgtttctgtcaatcagtttagcttggagtttatcaattttgattttttttccagtgaattcatgtttgattttgtaattttctctgtagttggtagattttctatttcattaatttcttatattttttaagttacattgatttaatttatctttttctttagctttttaagattgaacattaatctgttggttttcatttgtttcataatatatgcatttaaagttatacttcctctaagtattgcttagctgcatccaatgattttgatatgttgtgttttcattatctacagttcagaatatttatgagttgacttttttttttttttttttagacagtatctcactctgtcgcccaggctggagtgcagtggtgcaatctcggctcactgcaacctccacctcccaggttcaagcgattcttgtgcctcagcctgttgaatagctgggattacaggcgcacatcaccacacccagctaatttttgtatttttagtagagatggagtttcaccatgttgcccaggttggtctgaaactcctggcctcaagtgagcctcccacctcagcctctaaaagtgctgggattataggcgtgagccactgtacctggcccatgagttgactttctgaagtaatattttagtagtgagttattatggagtatataacttcatttctaaagatttagggattttttttttttaaaaaaccttactgctactgatttctaattagccttgttgtaattcgataacatattctgtaatatttctaacctttgaaatgtattgagacttctttaatggcccagtatttactttactttggtaacggtcccctgtgcatttgaaaaaaaaaaaaaaaaaaaaaaaagaatgtatattctgtagctgtggagtatagagttctaaaaatgacaggtcaagttggcttataatatctggatcttcagtatctttactgaatttttgtccactgttctgtcatttactaagagtggggtgtttttgcttggtatctctctctctctctgtatttacagtctttgttgtaagcagcatctaattaggtctggctttttaaatgctcagctgaaattctctgtcattttatgtggagtttcatccatgtacatttaatgtaattatagatatggttggttttaagtctaccatcttggtatttgtcttctgttagtctcatatgttcttttccttctttgcagtgaatagaatatatgttcttattttttctatcacatttttagctctactctttatgttattgtttttagtggttgctctagagtttacatatccatttaagtttattgcaatctattttcaaataatttcctaccaaatttgcaaacaatagaataatcttactattatataatttatttctgctattctttgggcttatgttgtcatattgtttacttctacatatgctataaacttcatgatatattgggtccattttgctttaaataaccaatggtcttttaaagaaatttatacatatgtatgcatgtatgtgcatacatattatagaaaattgtgttttatatttattcacatatttgtcctttccagtgctctttatttctttctgaagtctgagcttccatctggtatcattttccttaagcctgaagaacttcctttcaggctggtctgctagagaccttctcctagcttttgcccatctgagcatgtctttatttcaactttgtttttaatgatcttttcaacagatagaagattctaggttgacagttttctttttttttcctttttttggcactgtacatatatcacatcattgtcttctcccttcattgtttctgatgagaaaccatcattattatgcttgttgcactatacattatgactcttcatggctgctttttaaaacaatttcctctttgagtttttgctgtttgactatatgtgtttagtatccttcttgggattaatgagcttcaatatatgggttggtatttttatttaattttggaaaattatgagtactttatgttttgatacacttttcttttctttctcttttaactccttaacttctgggactgaaaatgatagtatgttagactgtttggtgtagctccaagatctagggtgttgcattccagtctttcagtcttttttatttttgtctttcatttggataatttgtattgctctgtctttagggtcattgattctttctcttatcccaatctgctgccagcccattgaatatgtttttttttttctttcatagagatgggatctcgctgtgttgtcctggctggtctcaaactcctaggctcaagcaatcctcctccctcctcaagcaaacctcagtgctgggattataggcatgagccactgtacctggctaaatgttgtttttttgatattcaatttttgtttatagaattttcatttgttttgctcttatacttttcatctttttatgtttattgaccaattaaatatcatttgggtaagcacctatttaagtgtcttaacaatttttctattgagtactctgggtttttgttttgtttttcttactgatttgtagaattctttatgtattctgaattgcagataccttccttctgtactaatgcttatctttttagccctgtaatattgtgttttcataaacatacttatcaatcttttcctttatggtcattgatttttgtgttctgtttaaaaaaatctgtatctatttcagtgtcttatttatttatttgttagttttccataagttactgaggtacaggtggtatttggttacatgactaagctctttagtggtgatttgagagattttggtgcacccatcacccaagcagtgtacactgcaccatatttgtagtgttttatccctcgccctcctcccactcttccccctaaacccccaaagtccattgtatcattcttatgcctttgcatcctcatagcttagctcccatgtatcagtgagaacatacaatgtttggttttccattcctgagttacttcactcagaataatagtctccagtctcatccaggtcactgcaaatgctgtcagttcattcctttctatggttgaggagtattccatcagatagatagatagatagatagatagatagatagatagatagatagatagataatctcacagtttctttatccactcattgattgatgggcatttgggttggttccatgattttgcaattgtgaatcgtgctgctataaacatgtgtgtgcaagtatcttttccaaataatgatttattttcctctgggtagatagccagtagtgggattgctggatcaaatggtagatctacttttagttctttaaggaatttccacactattttccatagcgactgtactagtttacattcccaccagcagtgtagaagagttccctgatcactgcatccatgccaacatctactgttttctgatttttttattatggccattcttgcaggagtaaggtggtatcacattgtgtttttgatttgcatttccctagtcattagtgatagtgagcattgtatatcttctttccagaattttctcttcatgtcctaagtccactttctgatgagatttttttttcttactgatttgttctaattcattgtagactctggatattactcctttgtcaaatgtgtatagattgtgaagatttactcccactctgtgtgttgtctcttcactctgctgaatgtgccttttgcggtgcaaaagctccttagtttaattaagtccctactatttatctttgtttttattgcatttgcttttgggttcttagtcatgaaatccttgcctaaaccaatgtctagaagggtttttccaatgttatcttctagaatttttatagtttccaggtcttagatttaagtctctgatccatcttgagttgatttttgtataaggtcagagataaggatccagtttcattctcctacatgtggctagccagttatcccagcaccatttgttgaaaagggtgtcctttccccactttatgattttgtttgctttgtcaaatatcagttggcggttaagtatttgggttctctattctgttccattggtctgtgtgcctatttttataccagtaccatgctgttttggtgactatggccttatagtacagtttgaaatcaagtagtgtgatgcctccagatttgttctgtttgattagtcttgctttggctctgcgggctcttttttggttccatgtgaattttagaatttttttttttaattctgtgcagaatgatggtggtattttgatgggaattgcattgaatttgtagattgcttttggcagtatggtcattttcacaatattgattctatccatccatgagcatgagatgtatttccatttatttgtgttgtctatgatttcttccagcagtgttttgtagttttccttgtagaggtcttttgattccttggttaggtatattcctaagtattttattttattttaatttttgcagctattgtaaaaggggtttagttcttgatttgattctctacttggttcctgttggcgtatagaagagctactgatttgtgtacattaatcttatatctggaaactttgctcaattattttatcagttctaggagctttctggaggagtctttaggctttccaaggtaaacgacagtttgacttcctctttaccaatttggatgccctttatttctttcttttgtctgattgatctggccaggacttccagtactatgttgaagaggagtggtaagagtgggcatccttgtcttgttccagttctccgaggaaatgctttcaacttttccccattcagtatcatgttggctgtgggtttgtcatagatggcttttattacattgaggtatgtctcttgtatgccgattttgctgagagttttaatcttaaagggatgctggattttgtcgaatgctttttctgcatctattgagatgattatgtgattttttggttttaattttgtttatgtggtgtatcacatttattgacttgcatatgttaaaccatctctacatccctggtatgaaacccacttgatcatggtggcttatctttttgatatgttgttggattcagttaacaagtattttgttaaggattttagcatctatgttcatcaaggatatcggtttgcagtgttctttttggttatgtcctttcctcgttttggtattatggtgatgctggctttatagaatgaattagggagggttccttctttctctgtcttatggaatagtgtccaaaggattggtaccaattcttctttgaatgtctggtaaaattctgctgtgaatctgtctggtcttggacttttttttgttggtaatttttaaattaccatttcagtcttgctgcttgttattggtctgttcagggtatctaattcttcctgatttaagctaggagggttgtatttttccaggaatttatccatctcttctaggttttctagtttatgtgtgtaaagatgttcatagtagccttgaatgatcttttgtatttcagtggtgtcagttaatttttctctcttcttttcttggttaatcttgctaatggcctatcaattttatttaccttttcaaaaaaccagctttttgttttatttatcttttgtgttttgttgttgttgtttcatttcatttagttctgctctgatcttagttatttcctttcttctgctgggtttgggtttggtttgttcttgtttctctagttccttgaggtgtgaccttggaatgtcagtttgtgctctttcagctttttcaatgcaggtgtttaaggctatgaactttcctcttagtactgcctttgctgtatcccagaggttttgataggttgtgtcattattgtcattcagtttgaaatattttttaatttccatcttgatttcatttttgacccaatgctcattcaggagcagattatttaatttccatgtatttgcatggttttgaagattccttttggagttgaatcatatggtctatcttggaaaaagttccatgcactgtcgaatagaatgtgtattctgcagttgttggatgaaatgttctatttatgtatgttaagtccatttgttccaaggtatagtttaaatccattgtttctttgttgactttttgtcttaatgatctgtctagtgctgtcagtgaagtattgaagtcccccactgttattgtgttgctgtctctcttatttcttaattctattagtaattgttttataaatttgagagctgtagtgttgggtgcatatatgtttaggactgtgatattttccttttggacaaggccttttactgttagataatgtccctctttgtctcttttaactgctgttgctttaaagtttgttttgtctgatataagaatagctgcccctgctgtgtttcatgtcgatttgcatgaaatcctttttccacccctttaagtttctgtgagtgcttatgtgttaggtgagtctccagaaggcagcagattggttggtgagtttttatccattctgcacttctgtatcttttaagtggagcatttaggcaatttacattcaatattagtgttgagatgtgaggtactgttgtattcatcgcgctgtttgttgtctgtgtactttgtgttttttttgtttgtttttgctttttaacttgtatttttgttttataggtcctgtatgatttatgctttaaagagtttctgttttgatgcgtttccaggatttgtttcaagatttagagctccttttagcagtttcttacagtggtggtttggtagtagtgaattctcttagcatttgtttgtttgaaaaaaactgtatctttctttcatatgtggagcatagtttcactggatgcaaaattcttggctgatcattgttttgtttgaggaggctgaagatagggccccaatcccttctagcttatagggtttctgctgagaaatctgctgttaatctgataggttttcctttataggttacctggtgcttctgtctcacagctcttaagattctttctttggtcttacctttgggtaacctgatgacagtgcacctaggtgatgatttttttcaatgaatttcccaggtgttctttgtgcttcttgtatttggatgtctaggtctctagcaaggctgaagaagttttcctcgattatttgcccaaatatgttttccaagcttttagaattctcttcttcctcaggaacactgattattcttaggcttggtcgtttaacataatcccagacttcttggaggttttgttcatattttcttattctttttccatttgtatttgttggattgggttaattagaagaccttgtctttgagttctttcttctactacttgtttcttcttctttcttctacttattcagttctattgctgagactttccagagcattttgcatttctgtaagtgtgtccaatgtctctcgaatttttgattttttttaagctatctgtttctttgactatttctccctttatgtcttgtattatttgttggatttccttgcattgtgctttgcctttctctggtgcccccctgattagcttaataactaacctcctaaattctttttcaggtaaatcagggatttcttctcagtttggatccattgctggtgaactagtgtgattttgtgaggctggtaaagagccttattttgtcacattatcagagtgggttttctagttccttttctcatttgggtaggctctgtcagagggaaggtcttgagctgaaggctgttgttcagattcttttgtcccacagggtgttcccttgatatagtactctctcccttttggtgtggatacggcttcctgtgagccgaaccgcggtgattattgtctctcttctgggtctagccacccagtgagtctgcctggttcccggttggcactggcggttgtctgcagagagtcctgtgatgtgaacctcacagagatctctttgggtctctcagccatggataccagcacctgttccggtggaggtggtcggggggtgcaatggaccctgtgacggttcttagctttggtttaatgctctatttttgtactggttggcctcctgttggaaggtggcactttgcagagagcattagctgtggtagcatgaagaggaactagcagtgcacaggaccctaggactccctggattatatgccctttgtcttccactaccacagtgcgtagggaaggaccatcaggtgggggcagggctaggcatgtctgagctcagactctccttgggtgggtcttgctgcagagtatttggggtgtctcctaggtcctgcagtagcagtccacttccttcaaagggtctgtggctcctcttgggattgctggtttgttcttgaagtcgatccagagctaaagttcacatgcaagcctctgcacactgctctgttagtccgagtcagagctgcaatctagtcctgcctctcgtctgccatgatcccacctcttgggattggcttctttttttttttctttttttgacagagtctcactgtcacctaggctggagtgcagtggcatgatttcggctcactgcaactttcatctcctgggttcaagccctggtgttttcttgccactatttatatgatcatgtggttcttcctgtttcgttaacctgataaatagcattgattgattaattgtcagatcttaaatcaggcttgtattactggtgtaaacagtttataaagtataattattttttatgttcttagattcagtgtatttagtatgttatttagtgttcttttttgttgttgtttgctttttcacccaagctggagtgcagtggcgtgatctcagcttccggttgcaagtgtttctcctgcctcagcctcccaagtagctgggattacaggtgcccgtgaccacgcctagctaaattttgtatttttagtagagacggggtttcaccatgttggccaggctggtctcaaactcctgaccttgtgatccgccctccttggcctcccaaagtgctgggattacaggtgtgagtcaccgggcccggacttagtgttcttaacacagtttggtttataattttcctttctttggtttttatatcaggataatgctggattccaattctctagaagaatttgataaaacttctgcaaaatcatcttgtgacccatctttgttttttctatgggaaatttttgtgtcatgaattcatattcttaaattcaataatagaattattcagatttaggttttttattatgtcagtctttgcccatcatttttccagaaatttgccttttgcatcaaaatttttaatttattggaacggtgtttattatatattctttttaatgcctgtagtatctcttctgttattccctgttaattcatgataatgataattttcctctctttttttcttgattagtctttttgaagtttattgatttcatcagtttttttatataatgaggctttgggtttgttgatttttctccttcaaattagttttctatttaatcattttctccttttttctaatttccatttttctcagattttaatttaatatgctactcatttttctatctttgtaaggtatatttttagatcctttacttttaacctgtctttaaaaaaaagtttgaaggaataattttacttgtaaacagcattagttgcagtcctgaaattttaatattattaggttttcattttcttttatttgaaaagtttctaattttattgtaatttatttgaaacaggtgttatatagaaatatattgctgaattcctaacatttctagttacatttctaattacattgatttcatgttaaatccacttatgtcagaatatattctgtataatatctgtctcttgaagtttgatgatacttgctttatggcataataaagatgcacttgaaaagtgtgtttatgcagttattggctatagtattgtagcctttaggtcatcattaagtcaagtttattaatcatgttttcaaattttctctgtcgatttttatgtgcttgttatatcagttattaagataagtgggttaaaacttatcttttatttccacttttatatgccttttctaagatgtgtatttttaaggtagcatatagacggacttcagccttgaatttcataataaacattaacatggaaaaaatgtacaaaggaaaccatctgtcagtgagatatgctgaccacaaaggaagggagaggtgcaccacattgctctaatgccttacaccattcactcttcattggagttagctgggactcaggattaagcactaaccgaagtccaacacaggtaccagcgctggatgtcaaaagatcagattagcatatgagggatgaaaggagtgattaatgtatcccaagggaaggacaagtttagaaaagcttagagaatgtttgtttagatcttgtcatacacagaagacataccaacactccataaagttctgatttaaaggcattcactgggttggatgaggtaacaaaaaaaacagatgtaaagtagagatctccccaggtgggacctgggaaccagagttgacctctgaactccctgaggcaggtgagatgctacagtagcacataatttacatctgctgacttagtgctgatgctgtcagtatttgggcttaggagcttacatttaagtgtcttccatattgtcccttttgcttctattcatcctgtaattgaaggtaatgataatgatttgacagaattacatttttatttcttaatattcctgtcggtttgtataccctgttcataattttagtggctccagaagaaatcctgtattagccaggagcacaacctgttatgttagtcatttcattgagaagtaggttttatttttttttgagacagagtcttgctctgtcacccagtctggagtgcagtggcatgatctcggctcactgcaagcctcagcctcccaagtagctgggattgcaggcatgcaccaccacgcccagctaagtttttgtgtttttagtagagacggggtttcgccatgttgcccaggctggtgttgagctcaggcaatctgcctgccttgacctcccaaagtgttaggattataggcgtgagccaccgcaccctgccagttttttgtttgtttgttgtttgtttgttttttgttttagtctcatgtgttcccaaaataaataagtagaagaatttcttctgccaccctatcagtaaacaccttactgatcaaaaactagtgggcaatttttttctctctaaatcagaagaaaatcttgccatagtctctcaccaagaagtgaaccctgctttgtttattacacctaagatttgtcttctgcgaagtcagtaatcgtttaaagcaatctggatgatttgtacttatcacagcttagaacacaattcttcagtggcttacagcagaattaaaagctttccactttctgtgaggtgttagcaaccttaatgatcctgattttatgttttaataccataaacaaatcatgaaaataccatctactgattaagtgacctcatgattaaatacatatttcatcttgtacctaatagaaggttgacagagagagggagagttcaaatgaggaatagaatgagccagaaggatggatggcatttttatttgcagaaaataggaatacatttttactgctgtttatagaaaacatgttcgttggcacaagcttcacaaagactagcaggaacagaatcccacaaaatgaagtttaaagtaaatagtggatagatacctaaaaatttctccaggactcaaagcagtttggtggttaaatgccaaacaggcattgtagttaaaattatgcttgttaaaagaatatgttccattctgtaatgttcggatacaaaattaattctagaagagtctgccgggggaaattttatatttttcactaccattcaaatatagatattgaatatgaaatgtattcaaccttcattttgtgtcctgaggttttatatatttattaaaatgtggttcttctttttttcttaagtcattttgttttctgggtctagaactcatctttttgtaagacttttagatgtaaacaataattcttaaatcaatatgtgttgccatgttctattagcaaatattttaagactttttaaagatcagctcttctttttgagctgagaagtctgttatttcctaattagctgggagctgggattggtcacctcactgagagaccagatataaccacacacacacacacacacacacacacacacaataaccttaaaaactagagaattcagaaaacaaaaatataaaaaatacagatatatttcggtattaaatgctttaaaaatatgaatcgtgtgtaggatatagtatcttgcagtgattaagagaacaggtgtaaggtcagtatgactgaattttctgtctttgaggaagttactgaacctctctgagactcagtttcctcctgtgtaaatggagatgataatccctcctatttagatatgagagtgaagttagataatggatgcgaaatacatatgtactaaggtttggatataagcaaggcttatgacatgttgcctcttcttggtttttgggagggcagggcaattgtacctttctttggctgtcagctaggtacagaaaattgtgggtggcctagtaggccagatgctgggctggtgggtcaagatgcaggtggtggtgctgagaagacacacacagaactgcctgtgttcccgcagcaagcaaactgtgattgcgacccgcaagtgattatttatgctgtactttctggttgtctgttaaacctgcctgtgaaaatcagttgtaagtcttccttagaaacctctacaaagtgtgcatgtattcctccttatatttctttttgtttgtttgtttgttttgttttttgagatgtagtctcactttctcgtccaggctggagtgcagtggcaccatctcggatcactgcaacctccagctcccaggttcaggcagttgtcctccctcagcctctcgagtagctggggttacaaatatgcggcaccacacccggctattttttttttttctcagtagagatagggtttcaccacgttggccaggctggtctccaactcctgacctcaggtgatccacccgcctctgcctcccaaagtgctgggattacaggtgtgagccaccacaactggcctctccttatatttctatagcactctgtactttctgctgaagtcatgtacttttatgtaagacagtgaactttggaggcctcagagcatgtataacgtgaatctgcacgtcccccactgttctatccttctcaagaagcaagcgttgtggtttcagagtaggtagggactaacttgagttgcactcatatttcccgaagccatagaccgtgatatcacatgtttcctaataaagatgaatactgaaatgagcatcatccagtgtcttgggtttttatgcttcttgcttttgtttttatatttcaccttttagaccaaggtaatttccaaagataatgtcaccttgctgggtgaggtgatgggtaagtgaacattcacagacccgttagcacagtgccttgcacgtgctcatacatgtcatttgctattgttatcatgtgcctctgaaatagacgtttgaaatactctcctaatcgtggaaaatttaatgcatatccaagtgttaaagctaaaaaaaaaaccctgttagacattttgacttgttttcttgttatcaaacctcttcctcagtgctactaaatgtaagagaaatagaagacaaaaatttctgacaatataaaattcaatatattgtaataaaaagaaaaaagtctcaggtcttttaaaacatgtaaaatttaattacatttctcttatctattctttattgggcatgttaatgaactctaatttcttatgtaagtgtgtttaggggaaaaattagagaaaaattcagggattttatcctgaccaataaaaagctagccgtatgctggggacaagctaaaatgtagtggctgacaatgcatgctcctcagctctgtgactggtgctgtgtgttcttgatcaagttacttttttctccctgtgcctcagtgtctccatctgtaaaatggtgtagtaatactacttcatagggatattgtgagatttaagtgagttgatacatgtgtatttaatatacagccagatatacaatcagcattcagtaaatgttagcaaaggatagataccaagagagatgactccccacttccaaaggtgaagatttaaattatttcctttttttctgagcactgaatttgcaattagtcacttccttatcccgtgcatagctgaggcttctgggttccttgtctatgatgcctgcttgctcatttcccgcataacatcaactgactcatgatagtttgggattcaagtcctactctaaatctcagttttctcatccattaaatagggaaagtaataatgcctgactcatgaggctggtgagtgtttcaaaagaactaatgggggcctggtgtggtggctcacgctcataattccagcactttgggaggcagaggtgggcagatcacttgagatcgggagttcgagaccagcctggccaatatggtgaaacaccatctctactaaaaatacaaaaattaggcatggtgacatgcacctgtagtcccagctactcaggaaactgaggcaggagaatcacttgaacacgggagtgggaggctgcagtgagaggagatggcaccacttcactccagcctgggtgacagagcaagactccatctcaaaaaaaaaaaaaagaaaaaggaaaaaaaaacctaatggataagtttatatcatagtgtttggcacatggcaggcatgtaatagatatcatcaggaccacctatacttttgttattcctttgtatggaaaagcagtccctggtactacaataagtctttcagagaaaggagtgtaatcctaacaacaactcatgcaagtatttttgaaaagaatacttgataaggaaaacctgcatctacttctgctatttcagacattgctacaagtggttggtgtggtctctgtggctgtggccgtgattccttggatcgcaatacccttggttccccttggaatcattttcatttttcttcggcgatattttttggaaacgtcaagagatgtgaagcgcctggaatctacaagtgagtatggaaactcgggttggtatagacatgctagctagtttccatttatgccataaattacagagaccccctgaaattcggcagactctgtcttccagaatttctctaacattaggtaattgaacgtattggccattatgaatcattgtgtcccttagagcatgtggaattgatagcctgcaacgtgcaactttgcatttggaataaggaaggagtgaaggccatatggggagtaatattctacaggaatgtcagcactgtgaagacagggactctccgtgtcttgtttatgacatactttcttacacaaagccggcacttaattaaacatttttcttagaaataaaatatttgatcatggtgaaactaatatagaaggaaaataattagggtattttagtatctttccccgaagatactaaaaatgacaaggaaatgagaaggatggagaaaggaaaagaggtgtcagaagcaatgtatttggtaaatttatgacttttgcaaactcacaaaatggctaggaagttgcagttttaaacgttttgaaaagataagcattgttattttcagttttccatttgttaccagcggtgcttgttctaatgtcacagtttgaggtttttccccctcaggctgtttatatatatatggacactgaaaataatccccttagatcacaaggagatatatcagtttagaaaacatatagtctgttgctctcttcaatttcctagttttggaatcatgaggaagacattttatgattaatcactctattccacatgtaatcgcagagatgaaataatccagagttcatctcattatgcatgcacaaattaacatatttcatcattgttaaacacaaactcataaggagagtagaattgctttactttttaataaaaggtataaggataatagccaaaaaataaagaaggaagatatttgtatcagaaaagctgaatgccaactcagctctctggaagccacgcctagaagagaagcattgagtcccatcattttcagtatcttctactgagtggtaaatagaagtttattaggacaaagcagtttttggttgtgacatatatatgtttctcttttaacaatgaacatttatttatttatttatttattttgagatggagtcttgctgtcacccaggctggagtgcagtggcgcaatctcagcttactgcagcctccacctcctgggttcaagtgattctcatgcctcagcctcccaagtagctgggattacaagtgtgcaccaccacacctcactattttcatattattagtagagatggggtttcaccatgttggccaggctggtcttgaactcctgacctcaagtgacccacctgcctcagcctcccaaagtgctggaactacaggtgtgtactgctgcacctggccaacagtgaactctaagaggcatttatcctaagtgtctgcatgaagtagaaggggtgacagtccataaaagttgtagaaaaacttttcagtcatcaagaaaaccagctaagctaccatgaggtctagtcttgtaaggttttcagtgggcaggccgagcagtggagtccacatgtgagtaacacttggccaataggacttgctagggaaggtagagtataattgctgggagaccttccttgtgcagatgactggccctagccagtgttgtgtgtggacagcaaggctgtgcaggcattttatggtttgggcccgctcatgaaatgaagttattcgagagtcttgttttaagggagtctgtggtatgttgaaggaagcagccgttccagaaggaacatcttgacgtagactgtctgtgtcaacagccacatccgatccatgtggatgtggagaaaagggggacttaggaaggctttttgggaagcagtgtaggattttctctcttgaatttcagcatgtaccttaaagcttgaaaacattttcatgtggagattctgacaaccttatcatggcccctgaggcatatttgggtgttcctgtaccctgattggctgtaacaatgagttaaatatgcaggattctttctccatgcgctcagatcaccaaccacgggggctggttggttggaactatttactgttattcgtccagggtggtgggtctctgccataagtcacaattccactgtttcttctcagcagcagtggatcgtggtttttgagaccactgctggagtcaccagctgaaagcaggatgcctgactgccgtggcagggaggcataatccagcttcatccttgtgccctcaaggatttccaaagcctctgttggactgcctcactgcccctggagtcatagccggcatttaatgaatcttaataacaaatctgggccgggcgcattggctcacacctgtaatcctggcactttgggaggccgaggtgggtggatcacttgaggtcgagagttcgagaccagcctgaccaacatggagaaaccccatctctgctaaaaatacaaaattagctgggcgtgatggtgcatgcctgtaatcccagctacttgggaggctgagacatgagaatctcttgaacccaggaggtggaggttgcagtgagctgagattgtgccattgcacttggcaggctgaggcaggagaatcacttgaacccaggaggcagaggttgaggtgagccgagattgcaccattgcactccagcctgggcaacaagagtgaaactgaaaaaacaaaacaaaacaaaacaaaacaaaaaacaaaaacaaatctgcagttgatcattttatgtttgcaagagtcaccacatcaacagtcagtcatagagaagtacataaaaaccggaatggcatttggctgcatttctctagccacctccactcatttggcaatgcctgaaatgtgaagccgtatccaatttctgtttgtaaagatattgtgatccactagcaatgtctgccatgagtgtgaacccatgtgtcccatgttcgttcatcctagattcccagggactttgttcaatgcaaggtggttaataagcggcacaggaatagggaaggctgttggatattagctaagtaagattaggggatttttaaagcgtgggctacaaagtctggagaaaaatgccctcaggatgcatgagaacctttaactctgattattctctctctctgatactgtggctcagattaaaagcacagccttggaaataaaacagatatgagtccatgcccaagtctaccacttgcatggcttactttacttaaccttgggcagtgttttttttgttttcttgtttttgtttttttgagacatagtctcactctgttgcccaggctagagtgcagtggccgtgatcttgggtcactgcaacttccacctcccgagttcaagaaattctcctgtctcagcttcctgaatagctaggactgcaggcacccgccaccacacctggctaatttttgtatttttagtagagacagggattcaccatattgatcaggctggtctcgaactcctgacctcagatgatccacccgcctcagcctcccaaagtgctgggattacaggcgtgagccatcgcacccagctttgggcagtgttcttaatgcctcaagtctgtttttccctctgtcaaggatataatacttatgagggttgcctgagactgcttggaaaccttgaacatacgggaactaccagaggcactatagccttgagaattaaaaccgtaataaccctgagaggtcctacacagcgtcagtaccccgcagcaagactggtagaccagatctttgcatgcagttccttttcatgttcttttcagagaagacagaaaatctctgtagagtgtatcctaaagagttctctgtgttgtggaaaacacagctattgatcttgtcaaatggcaggttgtgtcagctggaaactgctgactagtacgttagagctgaggcttgacatacgctctccctcggtactagttagttgtctttgcagacctcccagctcaccctcactggcagccctctcagccctaagtggggtgtgaaagagaaaggactatcttttcaaaggttacaaattcctgctataccttggtaatttggtgtgaattaaagtgtgtagcttgatcttcatgatattcccgggtgttccaaaactgggatggtacctaatttctcaagttgaccctgccctgaaacccagttggagataaaacagtggcgacttggatctcctgatgaggaaagcaaatgacaatagctatgttgaagaatttcattccaggactctgtccccatgactgccacatctccttccttctcagtggcattctctatatgatggacgcgttcagtcacaacactcccttgctctggaggtaagagcatggttctagtgatttctctgaactactgggagccaaactctttgtagggtaatggggcctggaattctcttgtgttattcctaatatcaccctgtttccagattgaccaagttccagggtgcaggctagaacatctggggcgtgcagaaaagacctgtgcagaacatccagcctttctcacagaagcaccagctcagtcctcttacggtcatgtcacctgccaccttcccagtgcaaagttacaggttatagagagctgacacaaataacgagtaaaaggttactctgtgtgttgaagagtcatctttttgttttcttgcttttgtttttgtttttttgagacatagtctcgctctgtcacccaggctggagtgcagtggccgtaatcttggcttactgcaacttccacctcccgagttcaagcaattctcccgtctcagcttcctgagtagttgggactacaggcgcccgccaccacacctggctaatttttgtatttttagtagagacattgtggatgccagagatagagaatagagagaaccctgtggactcgtgggaggctctctgcctgaattagacataagggaagtgccagatcacattctggtggagaatctatgctcagggaaggtctggtttcttaaagcactatcacaggagagggcccctaaaagttagtgcccccagtcagatgtatcagtcaggaaagagccccaaggatgggtggacagtcagtaacctgcagctccatctgcctttccccaacccagggcttcccactttctcagaaccccagtgtctcatacctggcggcctcctccttcacttacggaactgtggactctggactctgatatatgtggaacctccatgtcttggcacactggtaccccagcacccccaaatgtgcagtgtctgtgtgtacagtgggcacaccagtattgagtcagctgtagcacagatcggcatggggatgctgtactaagagtccgacccagaaaatgagttagaatttttctcttaaattttccactccctccgccccttacccccaccctaccccctcatcattttataaaacatatgtagccatcgcctgtaagatagattggccacgcacggtggctcacacctgtaatcctagcactttgggtggctgaggcagcctgattgctggagcccaggagttcgagaccagcctgagcaatgtgacgaaacccagtgtctacaaaaatatataaaaattagctgagtgtggtggcacacctgtagttccagctactcaggaggctgaggtgggaggatcacttgagcctgggaggttgaggctgcagtgagccaagatcatgccattgcactcaagcctgggtgacagagtgagaccctgtctcagaaaaaaataaataaataaaaagttatcttaacttctcccggacaccccatgttttactaacagggtcttctctggagctacacaggtagcaaagtcagtgtttgctaattgcaaagcacaaatcatttatctcagaaaagaatgctaaagacattgtgctaggtcacgtgtccccaatatgcacctgccccagtatggcctcattcatggtagacaattgaaactctcaggaattcctccctgcccaaaagtagctagccctcatgtccgctgctatgcaggagggaggtatgtcctggtagtcatcaaatggtgtgcttgaaacacagtggcctgctggaaatggtggctcacacctgcctgtaatcccagcactttgggaggccaaggcaggaggaggaccctcatctctatgaaaaactcaaaaattatccaggcacggtggtgtgcatgtctagtcccagctacttgggagcctgaggtgggaggatcccttgagccttggaattcaaggtggcagtgagctatgatcacaccactgcacccagcctcggtgacagagtgaaatcctgtttctaaaaaacccaacaggccaggtgcggtggctcacacttgtaatcccagcactttgggaggccgaggtgggcagatcacctgaagtcaggagttcaagaccagcctggccaagatggcaaaaccatgtctctactaaaaattcaaaaattagccggccgtggtggtggacatctgtaatcccagctacttagggggctgaggcaggagaatcgcttgaacgggggcaggtcggggcagggcggcaactccctcttaaaaaaaaaaaaaaaaacctacaccacacaaacaaaaacacaatggctcaaggaggagcagctggtcagggaacctcatgggatatacagctcccaaagaaaaaagaagtcacacaaaaaagaattcacttcttaaaattctgtagagatgaagaacccactgagaccacagtcagattctgaaaaaagcaatggcacatgccatagagacctccagagatcaggacaagtttgcagtggggcagtcataaaaacatctgtccggttcctggagccaatgttaagaccattcagagacatcttggctgggcaggttccttgggcacggcacaagggcattgcagcacccaggtccatggagatcacaaaggattcttccagcatgagtgggcacagaagattccccaggatgcagggaagtgacagtctgtctgttcagctcacttactgagtacatgtcctgtgccaggtaccatccagttaccagggtgcaatggctatagctgacccttgagaaattagcaatttaaggtggggacagacacatagatggctaattgtaattcagtatggtgagtactcttaacagagaattgttttttaaaatcctactggctcaaaaatcagaaaaatatcatttctgcccaaagagatgtcagggaagaatatggagaggaggaagacctggaaggctgagttaggaaggaaagaattataactagatttgaatgtggtcaaaaataggacgtttatttccaggccccttatgtgaggtgtggtttgagcaggagcttgagattccagtggcagggtcccctcaggctattgggaacatttatttttccttgaaacatgaagcctcctaggaggcctgcgtgtgctgtttggtggatacgtgaccatggagcttgggaggccgcaggtttttctgcacccagcctcactgagctgttttctctgtctgtttccccagctcggagtccagtgttttcccacttatcatcttctctccaggggctctggaccatccgggcatacaaagcagaagagaggtgtcaggaactgtttgatgcacaccaggatttacattcaggtctgtctatttctggaaatggtttcaaaggacaggatctgctgctttttctggcttaacctccctggtcagatgagcttgttggcaggcacctcagggcccagccccacatcctgctttttttgcgcatcttcgtctccttccccatccccttcttctgggcttggactcctctcggaatcgctgctgcacgttccctctttgtgccccctgactggcttgtggggtctttctctcaccatgcatggcctgtggctctgtcagcaaacgttttctgcaaaaggccacctagtaagtgttttaggcattgtgtgccattattgtctctgttgtgatgaatcacctttgccattgtagcacaaaagcagatgtcaatacattggcaaatggctgtgactatgtttcaataaaactttatttacaaaaataggtgctggctcacgtgctgtagtttgctgaccccttgcatcagtgaagattttttcaatatggaaatttcagaatttccaccaaatctacaaaattcagatccgtcattccaatctttattttatcttgctgattaaatggcactttaaaaaccacatgctaaaaggaaaaaagaagtagcaaacaaacaaacaaaaaaccacatgcaaatcctgttagtgaggtcatttttatatagcccctaagcaatagtcagtgtgtgtgggccttctggtaagtatggaaggtgggagaagtaaaatgggagctgcactctgctgttggagaactggaccagtcaaccatgtgtgtgggtggacttcacatgagatgcacttcacataagtatttctcagcactgtacgagatgtccttatgagactacttttctcagttctttattaggatcatatttctgggtttcatattcgcatgcatcctcacagaaagccacggtaccattgtgtatgtgtatcgtatattgcctgtatcacttagctttctggaatctaaaggttccttgggtgtgcaactttggtgccctactccacgtaaaaatcttctgtagctcaactgagcaaaacacactcgcacattcatctacttattcatcatgcttattaccgttctcaaattcagaggcttggttcttgtttttgacaacgtcccgctggtttgccgtccgtctggatgccatctgtgccatgtttgtcatcatcgttgcctttgggtccctgattctggcaaaaagtaagtacagatgtgaataactatgtatggtatgattacaatttatagctgtgtttacatttattaaactcttgccaccttgtctactactacatactgtttggtcctaatgaagtgtattaaatcatttacgatgtgtcaggttgggtaggattcagcagtgtgactcatttaaggagtcatttgggtcttgatgggtttatctttctttgaatgggttgagatatgcgcagttgagattctgaacattcctgttagtgtctggtagttgtgaagagaaaaccaatttattctgaagcattttgtccaacttagggtgttagccttccaactacttgaatctattgtttgtagttgcctggtgcatgaggttgagaaaaattctcagcaggagagtattatcttcaatgccacatgggtaccagacaagcagggcatgagttcaccaccccagtaacaacagtgcattacaatacttaataatggaacaaggagagtagtattttagtctcaaaagcattggcacttacaacctattgctttgtttttagtggtttccagctgtggttgcattgcacgtagaaagtggaataatgtaatgagctttgaaaccataataatgaatgtctgaataatgacattatttcttgcgtttgtaatactgttaattaaatctatgtcgatcctgttggaattcataaaatcatctaaaaatttttctaaatatacagtgttgttttccccattgtatcttgatctcaagcaacaaatggtaaaagtatagctattaatgtcattaaatgtgaattgtttcaacattatgaagggttcctcttggtaagtggcagaaggagccaggcttaggtttgaagtgagactgactttattcccttcttcctccttacgtagctctggatgccgggcaggttggtttggcactgtcctatgccctcacgctcatggggatgtttcagtggtgtgttcgacaaagtgctgaagttgagaatatggtaatatttaccttcacgttctttgccttttcaatctccttgccattaaatggtataaaagccattcttatgtgaaatattaaaactgttatttttacatctttggtaacagattttttttaagatccttcctccatctgtttaaagttaatgtaaaataaaatatatacatctttttctgagtcttatggttgctctgtcagcaggttttatattcatcacaagctttttcagatgtaataaatgtctctgtaggagggaggttctgaaattctagagaggatataagctaatttcttagaagcctgtttaattttgtttcttaacgggtaattcctcattcctgttaaaagaagacttcagtattttgaggttgtttacatttaattaataggtctcctcctctcccagttctagcgtttactctcttatgtgaagaacagaagggaggaaggccttaactttgctggtgtgccttcttggtacagaaagcctcctcttggaatgtgctagtcaaagggtattagtttatggtggtataggaggctttaatcaatgttttaaatatttgcaccttcccctaaagagagaaaagcaataaacataaaaatcttttcattcttctcatagagcaaaagttgagtaattattacagtgaggtttcacgatcatccagtttaactgacctatatcaggtgatccttctatagcaaaagattcttttttggggggtaggggtgaggggacagagttttgctcttgtaccccaggctggagtgcaatagtgcgatcttggctcactgcaacctccgcctcccgggttcaagcgattttcctgcctcagcctcctgagtagctgggattacaggcatgcagcaccacgcccagctaatttttgtatttttagtagaaatggggtttcaccatgttgaccaggctggtgtcaaacacctgaccttaggtgatccacctgccccggcctcccaaagtgctgggattataggcatgagccaccgcgcccagccagcaaaagattcttgattggtggctctaagtattcctttcctttacaaaattttcttgcctttaatacaggtgtgagaagtggataaataaaatctaagcattcatttctctcattcttttaacctaatgttgaaggaagtggttgtaagtgaaggtgttacacaatgatgaaccaaatttgtaagtcatcaacatcctcgtctgtctcctgcttctctccttaaccaactatgtatgaaagaagaacatttcattgacagtctcttaacatccataaatgacaccctccattcctaacccctcgggatgtcatatttctttcagatgatctcagtagaaagggtcattgaatacacagaccttgaaaaagaagcaccttgggaatatcagaaacgcccaccaccagcctggccccatgaaggagtgataatctttgacaatgtgaacttcatgtacagtccaggtgggcctctggtactgaagcatctgacagcactcattaaatcacaagaaaaggtttgtttaaagcatcttgcctcttctcaatttcagaagatttagtgctatgtctgcttgttgtcttccttgtggatgtgtgaaggggatcagtgatacgtaggttaacctgtcttggaaactaaccatggaatgtggataccaacattcttcctgctgtgaactgctttactgtggttggtgggccccgggcctcagggctggagcttaacgtgacccaggatactctgtttgatacctaattactcatggatttttccaggaagtcaactctggctttgtgtcaccaacactcctatgtctaattctgtcgatttttactttgttgcatttctcaagtccttctcttcccctccactgctgcagcttctgttccagcctttctcctcctccatcctttggtcctgtattagtctgttttcacactgctgataaaggtatacccaagactaggcaatttacagaagaaagagaggtttaatggacttaaagttccacatggatggggaggcctcagactcatgatggcaggcaaggaggagcaagtcacatcttatatggatggcagcaggcaaagagagtttgtgcagggaaacttccccttataaaaccatcagatctcatgatacttattcactatcatgagaacaccacaggaaagacctgcccccatgattcaattacctcccaccgggtccctcccacaacatgtgggaatttaagatgagatttgggtggggacacagccaaacattatcaggccctttccttagtctttcttgccttcctttaacctgtgctttgctttccctccccatccctttcactgcaaactctttctttctgaaacagtttgatcacagatctgatccttacgtggccagctccttgcctgggtgagcagctgctgcaaccttaatgagccccaggcccaccctgacaggacctgtctggctctcagcccccaagggaagacccagatcccatgaccccatcagtgctcagaaatcagacccccaagctggaggcagtaaaggcaccagagactcactcaaagtcagtgaaaggaaaacaaggagggacttgggaggctgtgtggagttttgtgagctggttttgcaggtatctaagtctgccagcaatgtccctttcacatgtgtcctggggcccttggctaggactaggacagcctcttgaaggaactaaaccagaacaaacaagacagtagaatctcttccccttcccacccctctcctcacaactcccaatatggacacacatcccctgcactcagggtgccctccttctccccaagcacctgtctgccttgctcttagtgtgcacccttttcagtcagttcaagcatctctgcctctttgaaactatattctccacatcatcttatagattcttttttcgagacagagtcttgctctgtcgcccaggctggagtgcaatgtcacattcccggcacactgcaacctccacctcccaggttcaagcgattttcctgcttcaacctcccccgtagctgggattataggtgtgcaccaccatacccaactcatttttgaatttttagtagagaccaggttttgccatgttggccatggctggtctcgaactcctgagctaaagtgatccgcccaccttaacctcccaaatcatgcctgggattacaggcatgagccaccatgcctggccagattctttaatgtaacatctgtggcccacccgtgttcataacagcattatttgcagtgagcagtaggtggaagcagcccaagtgtccattgactgaggaatggaaaaacaaaatgtgatacgcacgcataatggaatattatccagacttaaaaagaaggtgattctgacacatgctgacatgtgtgattctgacatggatgagccctgaggacattatgctgagtgaattaggccagacacctaaggacggtgctataaggttccacttacatgtgggtacctggagtagttagtcatggagacagaaagtaggatggtgggcggcaggggtaggggaatgggcattagtgtttaatgggtacagagtttcagttttacaagataaatggagttttagagatggatgatggtgacggttgcccaacaacatgaatgtatttaatataattgaactgaacattttcaagtgattacaatgatcaattttatgttactttaccacaatttgaaaagtacataaagtaccttttgtgtattttaccacaatttttttttaaagaagatagcacgatggcatgatttataattctgggtcagcatctcacagtaccctgagcctcttaaagtcagggagcattttatattcgtctctgtacccccagaaaatggcatgctgcagggcacatggtgggtgcctgtgagagtgagggatatgcacatacaaagaaatcggtaacaaaacacacacgacagctcccgcaacagcccgttctctgggtgtcagtgttagatactttcggaactgctcttttcagctcttcccaccacagacccatttaatgggctatagtcacaattgaatgaatgatacccttgtgccatgaaaaataaatatacagcttcatgcccagaagatacccagggaaccagcaaaagctgattcctcattatctacccagcatccttgtgagtgtgaagaaggtggagaccactctgcaggcagcatcagctgtagctcttgctgaaagcaaggtttctcagattctgttcctgatggggccagggggatgtttgcttactctcagacaatgtgttattaggtcctacctgatcaacatcattctgttagtttggccacgttgtaaatttctcttgtagccataaaatatgaattatctacaaacctctgttgtacacattagaaaacaacctatatccccacattgatatcgttccatcttaaaacccccccaaatcataggattacctgagatctgaaatgtgggagggtaaattgaatttgaagactgaattatagctacaaccacttggcttttgagacagagtctcgctgtgtcatctaggctggagtgcagtggcgcaatcttggctcattctgcctctcaggttcaagcgattctcctgcctcagcctcccgagtagctgcaattacaggcacccgctgccatgcctggctaatttttgtatatttagtggatccggggtttcaccatgttggtcaggctggtcttgaaccccgacctcaagcaatccacctgcctcgacctcccaaagtgctgggattacaggcatgagccactgcgctgagccccacttggcttttgatttgtgtcctggttcactcacagtgtcacctttagcttcatttgtgtattcaggcaacatcgtggtgagttggttccactttttgtgtgtatattcagggttcgtctcctccacacctgcctgcagccccaggatctcaggcagtccctttgctgttggagctgatggagaggggcattgtttccagtggagatgtaggaatgtgtccacacagcagaaacagggcttcattctctttcaattgcttcatctgctcagatttcctggacattataacaaataggaaaaagtaataggcgctaagtaggtttctaattcacagaagaaatagcaagtaaaaaagaatgaaccaaaatgaggtatataccaccctagccttcctggttagattccaaacactggctgcatttttcatttagatggttttgtggttgatttaatcattttagagagtactgggccattttaaagcatttttcagctctagaggtagattgattttggtttgttatttttttttccttgctcaaagctattacttggataatacctttttacagatgtgtactagaccttgctatagtgtgcaaaaatattagtttcttatattctctccttttttacattaatgtttgctgagaaacccctcctctcacagccagattgtcccacttgccctgcctgcttgctcatctcaggggctgggatcagcatcacctgcctctgcttggaaacatgttagaaatgcagactgtcaggcccaccccacccttttgattaaaattgcactgggacaagaaccacccccactcctgcccctgcattaaagccgttgctctccggtaagcaattttggatgactaataatctgtgatttccaagaaacttcctttgacaacttgaagtggagctcgtggattgccccagcccctcttgtgactaaacatgagactctggatagtcctgggccaagtgccaccctacaggttttttgtttttttacttttatattctgcaaattatgaatttacattacaatttaattctgtgtgattccccatttatgcattcatttgcacctttccggtcatctctgtcttctatcacagttttcatcccaccatacagccttccatgaatagcagtctctgtcctcataccagctcagcacagcgatgtatttgagttccgttcactactgatgggatcaaaatgggtctcactgccccacagtacactaggtctgtctggcctgtttcccagttgccccatgtccaacgtagctgcaggcttctctcccagcctcttctgtgggtgccagttcttcctgtcaacagctgtaatccaccctaatacaggtactgtcttggctcacagtagtcagcttcttgtcctagccaccttcttatactcacgcatgtccacagcatgcattaaatgtatgacaataggaccagtatggcaggccagtgcctttaaagacaaccacacttctccccgctgtctcccttctagctacaccaagggaatacagagccaggctatccaagtgattgggaggccatcggcagaatgggcaagaggcaggcagacttgggtttccgtcccattccatcacttaggggctcagtcatttcaacagtttaatcaatcttactaagcctcgatttttctcttttgcaaaatgcaagtgatagggcctagccattagagttttataacttaaacaagataagaggtgtaaaacacctagcttattgtatacctgaacaatatggcaatagatttcccataaatgtttcaagcctgagttaaatgcttaactctctgggggatttctggttttttttgcaggacaccacaggtgatacaaataatacaaagctaattccaatataggccctgcccacagcacctgtcgcctagtagaagaaacaaagtagtatataaatgcctgctccgtaagatagaaggaattcgcagattttatggctgtttagaaaagatgttacgacagccattaagtgtgggagaaatccacacgaggggacaagtggaggattttaaggcaattacacatttgaaacagactttagaaggtggttaggatttcagcaggtcgaaatgcatgtgaaaggcacgatggcatatgccaaggctcgatgccgtgatttgatatgaagaattaggcatctctgagcatacaagggcatgaactaccaggtgaataacacacatgtgcacacttagtcctcacagccaccccgcaaactcaatcctgctcatggagctaaggatggaaaggttagttgggactgttttgtagaggaggtttcccagtgtggagtgccaggcagagagctgtacttcggaagtttaatctgagaaatgagtgcacggggaagacgctgaaggcctgaagccaattgattttatgtactgctggcaagatgtgatggggcaagtgattccactttgtagaatcaaactaatgttgattaaaacaaagttataataccctggttagtgaatttgcagggatacaggtgggagtagaaattggtatcatatttctggagggtagtttggcatcacttaacagcctcctttgaaaatgttcatacctgttcctcagcaattccacatctaagaatttattttaagaaaccaattaagattgcttgcaaagatgtagcttcaaggatatttaacatggtattatttacaatagcaaaatattggagcctaaacatccacaaataggggatttgttgaataaataatcatatatactgtaatattatgtagctactgaagtaaatacttttattaaataatagattataatattctgtgcatattctcattttgcctaaatatatgagattatatctttaggaacacagataactcacactcaagttcagaaagaaaaggtattaacatgtgacaaggattctgtcagattgctggggttgtggccagcatttatttatttttcgaattcacttgcactgattataactagcaaaagagtttttgtttttcaaatgttaatgcagccttgcactgaggtaatggctgcagaaattaaagtgagggtattgagtcaagaaacagaacaagtaatatttttagtgcttggagttgagaaaacctgggagatgggtgttttgagcctgtgaacttggggaagaatttattagattaaaatggaggagttagaagcagcagctgtttggaggggggctgttgggtattaatcatgttgagtttcatatatgaagagagatccaagtacaaatgtctaatgtttggccaaagagaccactcttggcccagttggggaaacaaaaagggtcaattgtaaagcaattaaaataaaactaagtgtctctttcgtgttatgaatgtaaacttactgatattagtgatattctccagaggatttggtgaaacccattggtgacttatgaaagacttggagactgtaattttgatgactttgaagagaactaaccagagccaggttttctttggttgccttttgctgattatatcctcacctgagtttggagtgcctagtcttatggttgtaggcagccctcagtgttccctacctttgagggggtccatcctggccatctactaataatggcctacaaatgtccaggtttgcacagatggatcagtttcccatagagctgataacatgagtgtccacacattctcaaaaacacagcatttgtctatgtctagatacgtgctgatttactgagaaacacacataagccccacacacacacacacacacatacacacatacacgtgtgtgtgtgtgtatgtgtgtgagagagacaaaatatagtcaggtatttaatatttggaagtttctctaccaaagaatgtgtcatcttaaatgatagaagtctggaaaaatgaatacctgtttatgtcacttgtgacacctctgtcttgaattaatatccctgtgaaattatctcagaaattcaattggtgtgtgtgtgtgtatatatatatatatatatatatatatatatatatatacacacacacacacacccggatgtacctatgtgcagaaaatggcccacctataaagtataaactaggatttaccataaggtagaatatgactccaaagttaataaaattagctaaagccactttttaaacatttacatttttatattttgtacatgtatctctttaaagataaaattccttgtggaaatgccattattgactttacagatatggaggtgttcaaatgaggtaatatgtcacatttccaacacatacccagtgcccagtaaagttttgcagtgagttttaaaatttattaaataatggacaaatgattgaaggcatgttttatgtccttcgttgactccagatttctaaataaggaggatgtcatttcttgggaagttcccaccaggaaatttgcagttgtgcaaggtcattggtgcagaggccagtttctgcatacagatttatatgcacacatgtgaaagccagtttttacaatatagacgtattgcccccacctttttaccagcattttattgattctttgatcgtattggcagtgcacttcatttctctctgtcttggctttaatagaggagtgatacttgacacaaatgtttctcaaggggcaattgtaagtattaattagtttgcagcatgccttttgaactgtgcagatgaaaggccttacgtaaatagcaaatgttacttgcgttgttgttgatctcattatgtgttttttggctactggttactactgtcttttaagacattgcaatcttcacttcagaattttgaaacattttgagacaaaatataggcagatatttaatatttggaagtttctctaccaaagaatatgttatcttaaataatagaactctagaaaaatgaatactcgtttatgtcacttctgacacctctgtcttgaattaatatccctgtgaaattatctcagaaattcagattactcagatcctgcaagcaaagctaatttaaaagttgccttttcatgggccacttaggagactgttgacttttcaatgccagtaaacagattctgttattttcaactcttttttagaacagtggatctcaaccaggggcagttttgccccagaggacgtttgtcagtgacatttttggtggtatcattttgggagtgtgctcctggtatctatgcagtagggtccagggatgctagcgaacctcccacaatgcctggggcagcttccaaaatgaagaagtatccagctcagaaggtcatcagtgctgaggttgggaaactcttagactacactcaatatagaatgtccttaattcttcttcaaaaaccgaaagttttctcctagttttgctgcatcctgtgatttttttctttttttttttaatcctgccgcctggatctctctgtaggttggcattgtgggaagaaccggagctggaaaaagttccctcatctcagccctttttagattgtcagaacccgaaggtaaaatttggattgataagatcttgacaactgaaattggacttcacgatttaaggaagaagatgtcaatcatacctcaggtatgcaagtcatagcgtccttacctgccccttttataagagctactatgtttaattgcttttactcttattgctttttttaattggatgaaaggattgttttttttttcacccaatagagcatgtttttatgagcagatattttcaacaggtattttcatcagatatccagctttgttcatttctctctctctctgtgtgtgtgtgtgtgtgtgtgatatttaataacttacgcagatagatttctgtactaagatgccacagttttatccactgataaccataaagaacaaaatgatgcattgtttgtttagcactggggaggtttctatggggagatgtggattttaagtgtcttaatgttggaattttgctgtaggaactaaagctaccttctcaaaggtgattacctccatgaagggctttctgtgaaactgtgggctgtgagagttaaaaaacttatagttggctttgtggtgacttttagaatctagctcttggtgaatgattttaaccaggaggttgttaaagaatagcaaggaagaacatgagagacggaattccatctctctgcacttctgcttcatcttccttacgatcaccattgtgagagtgtgtggtgtatttcacacagacccagtgagtgtttgctctgatggtggagggttaaattggccatcctttcccggactctgattctattttcttttcttctcctttgcagaagtgactctcaaggagtgttctagagattggattatttgtacaataagagctcctgtcattgctttggttcatatttaacataaattcagaaagtattcattgagtaattactattctccaggtgtatagtcatgagcaacatagaccttgtccccaccggcatggggtctgcagctgtggggcctcatggcttccatcggacctcacagttccatcctgtccttcactttaacacctccacatattcaactccagacatggctgggttttttgtttgttttcttttaatgtgggttttttttttttttaacactctatagtcattgaggggagctgaggaacacacacacacacacacacacacacacacacacacacacacaaacacatattctggagaagagaaacacagagaggagacaagagcaaaacccaggcagtaatgagctttctgggcatgtggatataaacaactcttcaatgcctgtttagtaggaagcatagagaacgtgccaccggaagggaaggagaatgtgatttttatcttagaccaagtacagttatggctatcctagcactgacctctaaaacaattggaatcacgcttaagtaatttggaggatgtgaagaaattggaaccctgtgcactgttggtgggaatgtaaaatggtgcagccactttggaaaacactgtagagattcctcaaaaaatgagagctaatgtgatccagcagttccacttccagaaagaattaaaagcaggatctcagagttttgtacatccatgatcatcacagtacccaaaaggtggaaatagctcgtgtccatcgccagatgagcagatgaataaaacgtggtctatccatacaatgggaaattattcagctttaaaatggaaggaaatcctgacagtctataatgcagatataccctaaagacattatgctaagtgaaataagccagtcacaaaaggacagacactagatggttcccctaatatgagatacctagactagtcaaattcacagagacacaaaggagaagcgtcgtcaccaggggctgagggaaggtggggggttgggagttacattatttcacataggacttcagttttgtcagatgaaaagcgtcttgtgaatggatggtggtgatggctgcaaaacagtgtgaatgtccttaagaccactgaaatgttgactttgaaatggcaaaagcagtaaattttatgctatgtgtattgtaccacaatttttatttttagtttaaagaaaaaaaagtatccctttcaggataaaatgggaagaaatgtttagcacgttagattccctctggctattagaagcatgacttagtgcttacagtagaaaatgagagtataaattctcaagttttatattttggttatgcccatctcaaaaatgggccaaatgaggtttacttagcagagataatgggcatgtctgtaaagtgcttttatatccttggatgaaaggttctaggaacgtgccctgcattactttgaatgaattcttaataagaaatagttgtgtgcttgtgtacataaaagaactctaaataaagaagtaacattagatgattcttggctctgccagtaatgttttggaagcacatctgagcaaagactaaatataccttgacatcaccaggttttggttcagttccataagtttccaccctacttttgagcaaatgctctatgtatgtagtccagggagcatgcttacaaaacactgctaagctttgtatccatgtagatcctgatctccaagcattgtaaacagagagactttcctttttaactttgtatggctttgtctccaaggctcaatgttggttgaaaaaaagaaacatagtctagagggactgtgcaatacttaatgcgacccaggaaatcattcagcagactgcctgggatagaatctttcagagcatcttctaagcgtcaacacgacccacatttttattccagcactgcctgatagaagtgtgacctcaatgaagtcacttagcctccaagcttcagtttcctcatccttaaaccaaacctagtaattactaacttacagatttgttgtgaagattttactagatgagaacatgtttgtggccctactctgggttctgaggaatgctcaatagtaactatttctaattaaggattggggtgcaggcctttagggcattattgctactcgctattctagaaggacttggctcattaacttctgtttctatttttgtgatccttggtgaattcctttaaccaaattgtttatgtaaatttaaatagtgaagcagcaacaaacaaaaaaaaatgtgcacaataaaagcctaagagagaaatctaatgtttttctcacaaggaacaaaaggtgaggctatcactttgttttctggctttcccccaccccggtcttagcggcattcctcaccactctcagtacccctctaatgctttgcatttcgcgagcaggcagccgacactggttctcagccctgcgtgagaacaggggcttgtccctcttagatccagtgctttagtgattgttgcgtcccagacaagttacacaagtagtgagaattcctaacatttgcagaatccttaacatggttatcttctttccttgccatctcccacttcgtcctcacagcaattctgtgtcatcctcatttgacacattttcaaactgaggcacagagagcttaacccacttaacaagtaaacagcagagtcaggattcaaatggaatttacggcgaatctcaatgccagatcctgagttcctgaaccctctcctagggactttaaacccatgttggctggcgcagtccccgatcagaactctgctttgcaacatgatttagtgtgggcttgtgcaggtgcgcacacacgcacagaaacaaaagtctcacaacacgatattactccatgcttctgcgccctgatattttctattatatccattctattctggttcatataaaagacataaggatggtcatgagtcacaaaagtgatttcatgatccattggttgcacctgccatttgaagaacagcgtgcctcactgtgctgccctgcttggccgggctcctgctgcctggcagcaccgtgcaaggagccttatgacacagtgattgtcactgcagccctgcagggtgtgtcaagttagccccattctccaggaaggaaactaaggctcagggagattaataacttacctggagatgaacagctaataagtagtagaatcagaactcagaactatccagctcaattttttttcagtaagtatttgttggacagcatgatatctcatcagacattttactcattgtttttcttttctttttctttttcttttctttttttttttttttttttttttttgagatggagtcttgctctgttgccaggctggactgcagtgacgccatctcagctcactgcaatatctgcctcctgggttcaagcgattctcctgcctctgcctcccgagtagctgggattacaggtgcgtgccaccacacccagctaatctttgtatttttagtagagacagagtttcaccatgttggccaggatggtctcgatctcctgaccttgtgatccgcctgcctcagcctcccaaagtactgtttgtttttctgtctacccaacttacaaaaggttcccagaagccataaccaaagagagcaaatatacagcaaagtcaaaaggtatcttttctttgaactttatctgtcatttggacagttaggtcattttgaaagactgaatcatttgtgatttatttttatttttatttttattttttccaaaaccttaagtgacactaagcactaaaagaaaatcattttggaaacagagtgtattcacttatgtgtcgtgggctagtgagcgtcagaagatcctccaagccttaccttgtgggagctcagagccctttttcttccgaggcctcgctggctgcagagggtggagaaggctggagacttgaggttgcatgaaggttactggtctgcagtgcagaaagcatacacccctcttcctcagaagtgagtggaaagccaaatggcttgtgtcaccacacactgatgtagggaatctaggaagtttgggagcaaacacagcaaaagcagaaccaagtgctgtttcttcgaatggccatgtttgggcaagtctagcctgcatggcttcctggggttgtgtggagcacgagtttctgtgttctctctcttcctggcttatccctttcatttatggttcagggtcctctgtcactcttgaccacaccattagcaactcaaattcgtggttaggattaagcagcaaggacctgggaaagaactggggcacagaaatgatgacatacatagtcaggggccagtcctggagtcactttaagaaacaaatactgggccgggcacggtggctcacgcctgtaatcccagcactttgggaggccaaggagggtggatcacgaggtcaagagatcaagaccattatggccaacatggtaaaaccccgtctctactaaaaatacaaaaattagccggacgtggtggtgcgcatctgtagtcccagctgctcgggaggccgaagcaggagaattgcttgaaccccagaggcagaggttgccatgagctgagattgtgccactgcactccagcctggcgacagagggagactctgtctcaaaaaaaaaaaatactgaattcttaagaaagtacccctcaatttgtattcaaaatttcaggaagaagcgaacagtgaaagtacttcctaaataatttccctaaacagaatagttagctgaaatgtatgcttcttttgtgttcatgacacagggtagttaagtgcacttcccaaggagaacaccaaggtgatactataactcagacaagagtttgcttaactgaaggatctggaaaaatggatttcaaaactttttttaggatggattatttactcctccaatcctactggcacaaataaatgttttcaactaaagagtaacttcatgcaaattcaacatttatgatagcaccctaacggttggcatacatcaacagctataatctttgttaggattaaacagattcatttttcatatttgatggacttttttgtttttctatcaaagcaaaagctaagtagtttttaagcagcccactgctcagacttctgctatcttggtatttctactccctgaattttcttcagtaagttgaaaaggttaaccagagtgtgtctctttaagacttctttttttattacctgattttctttcctttttttttttttttcttctttttgagaacaaaattcaaagggaaaagaggtaggtatgcagttactagtaaagaaatacaattggattctggcctaatgcagtcagtcctttgtttctgcacactgaagtcttcattaaggccatggaactgctccctggagctggtgacctttttgtatctaaagaagactttacacgacttaaattctttttcctttgttggtgaacaatgttcttttcttctgaaaatggatgatgatcagttcattcagaggctacagggtttttttaaaaaagaagggaaaccaagatttaaagtgacttcaggtgattccctgattcttgacacagggagatgagctgccctgaatcaaggtcttcaggatttgaaatgcacacatagctctgaggacatcaggcagcagcagttgtggctagctactgtgcagtgtatattttaagagagcgaagtttcctggtttgggacaagattagggtaaagtattgtgctttgtcctgctggtttgtaactccccatgcgatttttgtcttggtcccaggtcgttttaagtgtctgaaggttggtggatagagtttcttcttgagataatgagaggcttgtcttccagaagtttatatgaaagctctaggaagactgtaattttcagtcttgcagtcctgctgagggattatcaatttactgcactaaatgctttgattcagagcggccatttttaggtcacgtggtgggttttttttttgctgttgttgctgttgttttgagacagtcttgctctgtctcccaggctgcagtgcagtggtgctatcttggctcacttcaacctccacctcctgggttcaagtgattctcctgtctcagcctcccgagtagctggaattacaggcgcaccccaccctgcctggctactttttgtatttttagtagagatgggatttcaccatgttggccaggctggtcttgaacttctgaccaccagtgaaccacctgcctcggcctcccaaagtgctgggattacaggcatgagccaccacacccagcctggtcacatgtttaatatcagcttgtcttatgtgagtaatgaggacaaaattactactcagtatggcgcaaagatttttaaaaagtatgacctttaaaccctaatgaaaaacaggtttgatctggtgcttcatttaaaataatacgttgaacagaacattggttttgctaacaatggaggttaaaactcgtactaaatgacattttaaaatatataccatcaataaatatgtacttaggctactaaatcttttacattttacatgaagattgtatgtttaccataaatctctactctgaaatgttctgcctgcagaggttggaattttagagttgataattacaaagagtaagtcaagtttttatcttttaatctgaaataaccagaatgatggttattcccctcacaagaaattcccaccattgtttagatggttgttcaggttttggtcttcagttgggtacttttaagacagaccttacctgtgactcttggaatttttcagttccatttggtaattcagaaacatcatgtgaccagtgacttttcagtgcttttggaaggatattggccactttatcagctttatacacacaaagtgcccattttaccgtcgtttccatttttgcattttgctgaggacttatgtttttggtggcaccctgtctgccttctacatccatttcttcctcaccagccttaaaacggagacgatagggaaagggatgagagcctccctggatatcatttttctttatggctggagaaaaaagaagtctaccacctttatctctcttctcaggcttttgggaagtgaggaggaacagtcctgccccaaagccaggtgagatttgaggccagtggttcccttgagcatttctccagtacctgtggaagagtctcaccttctcttgttcccacatctgtgcatccccttgttctgcccctatccttgaaagcagttgatggggctcctatatccttatggcctggcttattagagccttccagaagaaaagtttgtgtttttgtttttttgttgtttttttttttaagatggagacttgctctgtggcccaggctggagtgcagtgacggaatcttggctcactgcaacctctgcctcccaggtcaaagtgattctcctgcctcagcctcccaagtagctgggactacaggtgcccgccaccgcacccagctaccagaagaaaactttcacacagcctacctaagcttatgttcaagagtctctcttgaaatccaggaagaggctgatattccaagagtgatttaattgtgggatcctcctgaagcagatgaataggaacagctatttaaattgatgtaattttattatgacctccttcacagtggttttcaggtaaccctgctttcttagttaataatgaggttctgtgacccaggtaaccttgatgcacagatgagataatgtggggattcactgatgcaaaatcagaggcaatcctttcataagtagaggtgcctctggccccagttgtttagatctcctttggccaaaaaaaaaaaaaaaaaaaaaaaaaaaaaaaaaaaaaaaaaaggatgtcccattctgacattggagagatttttttttttttttttgagaaggagttttgctcttgttgcccaggctggagtgcaatggcacgatctcggctcactgcaacctccacctcctggattcaagcgattctcttgcctcagcctcctaagtagctgggattacaggcatgtgccaccatgcccagctaatttttgtatttttagtagagacagggtttcactatgttggtcgggctggtctagaactcctgacctcaggtgatccacccgcctcgacctcccaaaatactaagattacatgcgtgagccactgcgcccggtgagagattcttaatcttggaaaactgttttgccaacggagaaaataatcctaatttattctttagtgcaaattggaaaatgcttaataatttggctttgaagcacaaactttaatcatgctgtcggtattttccagtattacatgattgatagttaatttctatagctctattcatgtagaccctcgaataattatatccttatatttcatgttaagctaataatatgaggaaaacagttttaaacatcaggcctgtatttttcattttcttcttaagatagcacattgcaacagttttgttagtgttcagtggtctggaaggtattttggagcacgctttgtacagtggaacattaatagatatgttgttttgtaaacaggcatttgtaacgctataattattatcatgaattttattttttggtatcttaattctattgttgatgttagcattggaataaaatgaaaaaagagtctgtggtaatgaccgtgactattttgggcacttaggaacctgttttgttcactggaacaatgaggaaaaacctggatccctttaatgagcacacggatgaggaactgtggaatgccttacaagaggtaattgataaaatgtaattatctttagtctgttagcctcagtatgtgtatatgtacctttacagcattgcaggaaattaagaggagcttagcggtttaactaactttgttcaccacccaggagatcattgaggatgtggatatgctataatgtgtatactgatgatgatgaaaaccaaaaataaaatttccctggttctcattttaattttcttcctgttgtccaaaacagtttggtgttgttttatttgtctttagcaatgcgttaaactagaagttgtatttatgagttgagatcctattagtgactaagttactctgcaccatttattttactgctctgttcttagggaatttctagtaagggaagaccatagtcgtttttcattaactaaaagatagatcatatttatgaaatgctttaagttttataagaaagtgtttataaaggtaaattatacagttggaatgggataataattatttattggcaaacgtgtaattttaatgcagataacagaaccctgcagcctctgctaaggttttatactaatatgttgatttctagaggatttttctattatttatgtggttcctgttgggccacttcttgaaattttctcacccttgcatgaaaaaaggactttgactattaaggtgctgttggtttgtgtattttaacactgattttctgtgttcagtctagttctgcaaatttgtgccagtctacttgatggcagaaaccatatcataaaactggtaatccctcacaggacctagtttagtcttttgcttttaaaaagtaagcatttaaggctgggtgcagcggttgatgcctgtaatcccagcactttggaagtctgaggtgggtgaatcactggaggtcagaagtttgagaccagcctgaccaacatggtgaaaccccatttctactaaaaatacagagattagctgggtatggtggtgtgcgcctgtagtcccagctactcaggaggctgagacaagagaattgcttgaacctgggaggccaaggttgcagtgagccgagatcgtgccactgcactcccatctgggcgacagagcaagactccgtctcaaaataaataaataaataaataaaaataaaaattggccaggcccggtggctcatgcctgtaatcccagcactttgggaggctgaggcggatggatcacgaggtcaggagttccagaccagcctggccaacttggtgaaaccctgtgtctactaaaaataccaaaattagccagacgtggtggtgcgcatctgtagccccagctactcaggaggctgaggcaggagaattgcttgaacctgggaggcggaggttgtggtgagccaacattgtgccactgcactccagcctaggcaacaagagtgaaactccatcccaaaataaataataaatattaaaagttaaaaaaaagtaagcatttaataagtatgtattaatatgtttgatctcttcatttcaaagaagtgtggagcctggacaacatggtggactttgtctctactaaaaataaaaaaaaaaaaagaattagccagatgtggtggcatgcacctgtagtcccagctactcagggagttgaggtgggaggatcttgtgagccctggaggtcgaggctgcagtgagccatgattgcaccactgcactccagcctgagtgacagagattaatttccctccattgttattgttgttgttttgaattggtgcatttgtcattgttgcccttccagggatagctctgaaatctttaatttatagatgggttttgactgtgagcaagtagacttggaggcctgacagtttcctgccagtaattcttgtttctgaaggcatgtcattgccgagtccttacacacccaccctgctttccagcaactgtggggattttctttttctaaattaggtcgattacttcacctcctgatacttgataagcaattgacctctgatttcatttaaatttgtgaatctctgtacagtgaaattctaggagagaagaggtgggtacagttaactgtcaaatgtaaagaaagataaggcaatcctgtgcccctaaactctcaggacaggtttaatagtgaaattgtgacacaccctaaaagaacaagaattactcataaagtgtttttctatttgtttcagagcttgagattccttgtcacagattctcagagagcaaaagtactttttagaaacacatttctctatgaacagtgaagaaatgttaattgggagatgatcctatcaaaaaccatcatggaacttgactgtaactcatacttatttcatttgttatagccaaaattaaaataaaatttagtagcctctcaagaattctagaaatgatgaggtggaagacgtggtaggtccccctcagaagtgaaaaattatcccatcattgtctttctctgggaactacttctttatggcattgtaggtttttctgtgaatgatttggaagagttactaaaaattagggtacaacattgtatttccagtgtgattccagctgtttttaatcattcttatattctgacacctgacctaagaaagacaccagagcatttacattgctcgtttgggggtaatgagacaaagactggaggagagtgttttcttttctctacagttttcctggattttccaaatttccttctgggtgcttgtattctttttaccttctagatggtcaggggaaaggctgagcactcctaacaaatgaagcattgtgagtttttcacaacaagtttcatgactacaagttgatggaggctgggggcattctgataggtcaggaattcttcccctggggtccgtggagtctgagggtcttctcgtagagaattccaaatcttacttcctggtgattgtaaacgtgaagttatgtgtgtgtgtttatcttgagagataccacacaagggattcagcaccacgactctggtgccattcaagtttctctggacttctctgtcacattctgtctctctgctgtctccatggttttcatctttcattgatatccagttcagaactggataactcatcttgctctttagtaggaaacctggtgtcatttggaaaccccctttaaagcagacaaaaaacatagtcacttggtttataaatgaggccaaactggaaatgggaacccaaactcagagggttggcttgagattaggacagcactgtcagttttttcctctttaaccacttaaaaggtggtttaaccacataaaaaggaaagatgccttttatttcttggaagttttttttatttcttcttacccatgatcccttggaaagggtaatacctgacatgcaaaagaagttcgggcatctggagggagacagtcctatggatgtggttgtgtcatcttggttatttttttatttatttatttttatttatttatttatttatttgagacagagtctcactctgtcatccaggttggaatgcggtggcacagtctcggctcactgcaacctccgcctcccgggtccaagcgattctcctgcctcagcctcccgagtagctgtgattacaggtgcctgccaccatgccggctactctttttgtatttttagtagagacggggtttcaccatgttggccaggctggtctcaaacccctgacctcgtgatccacctgcttcagcctcccaaagtgctgggattacaggcgtgagccaccgcgcctggcctatttttatttatttatttatttttttagacagagtcttgctctgtcgcccaggctggagtgcagtggtgcaatctcagctcactgcaaactctacctcctgggttcaagtgattctcctgcctcagcctcccaggtagctaggattaaaggcaagccccaccatgcctggctaatttttgtatttttagtagagacagggttttgccatgttggccacactggtctcaaactcctgacctcaagtgatctgcctgtctcggcctcccagagtgctgggattacaggcgtgagccaccacaccctgctcgttttttggggtttcttttctaatgctgaaagttatcgtaaatactgaaaaatagtcagctgtaaatggaagaaagtttactagaattagtgccacttcgggcccctaaataagagcaactctgtaaatataacgtaagtaatgattaaagatggaagtcgtttatgagaaaaaagtcaaaagatcacacagtaggtacaactaagaaaatgaatcagcagcacgttatttctgttaattaaaaaacgactttaatactgggatctacaattaagactaggttatatcatgttggaatagtggagaaattatctaatcatgttcaacattgatagaattgctgttagaatacaaggatgtcttgtattcaggacaccataagacagaaatctgcacatcagcagtgtccacatcctctgggccttctattccccacaccttgcacctccatttttcccaggcctgtattgcccagaatgggcttccgctcgcttcgttcacacttagctgtgttcttgttaaaggttgttcccatctgttgagttgctgtttatcacatttcagtctctggcatcagagacttcctttatgaaaagtcagaaagttcaaagaaacatggaaacagaatctgacatccacatgagtctctttgaggctttgaaaacatttatgatgatagaaatgggctgtcttagaaagttcagttcattattttcacttttcctgtagtttcctattttttctaagcttcaaaataacctgatacattgtgtttgaaatgtatagtttcaaatagtatgatttgattttgttttcctgaaattacaacctttattaaatgtagaaaacattaagttttctatattttaaagtataatgttatgatatcttggagccaattaattttataaggactgtttcttttacaatgacatttagtaagaaaattgtatgtaaattctatttctttgtggatttactttgtaatttataagaatagacatgtcacaaaatgaggtatccttgtgacttcatacattcaacagattatgtgtggggaggtttttacataggcattgtagactctggagctatagcagtcatcaagagagataaaatccctaccctcttagggcacaaattcttgtatggaagatagaaaataagaaaatgcatatgtaatataatattaggtactggtaaatgcagtgaagaaaactacagcagtgcatgaaaagaactagctgactgcattattaaaaataggcggctgggcgtagtggctcacgcctgtaatcccagcactttgggaggccgaggcgggtggatcacgaggtcaggagatcgagaccatcctggccaatatggtgaaaccccatctatactaaaaatacaaaaacaaaattagccaggtgtggtggtatgcacctgtagtcccagctactcgggaggctgaggcaggagaatcgcttgaacctgggaggcagagattgcagtgagctgagatcgcaccactgcactccaacctgggtgacagagcaagaatccacctcaaaaaaaaaaaaaaaaaaaaagaataggcagtatttcattgtccaggaaggagcaaaataaacaaaaagaatagatattatctattgatctcttacttggtaccaagcactattgtaaatgctacacatactgactgattttatactccaacaattttaggagataaacatcttcaaaataatcttagtatggtcttatttacaagttaattaggaagctaagatagagaaagttcaggaacttgcccagttggcagagccaggcaggactcatacccatgctgtgtgtcttcaaggctccatttttttattttaaaatgtccaaatcacagaacagtcaaatgaatagtattatgaacatacacatacccttctccaagattcaccatttgttaatacggtattttgctacatttgcttcctctcttatagcccttgaaagtaagttgcagacatttccatgaagttgcagttcatccttaaatatgtatgtgtacctcctaagaacaagaacattcaactacatgacataatacagttatttacctcactgaagaaattgaagagtaatgtaataccattgtttaatatattttcaatttccccagttgtcactcttagtcactgtgttccactgcctccattgtctttttctcagtgctaatacttaaccataacttgaatgtatgataacactacagagggccttcacattcatatcttttttttttaattgagacagagtcttactctgtcactcaggctagagtgcagtggcgcgatcttggcttattgcaacctccgcctccctggttcaagcaattctcctgcctcagcctcctaatgggattacaagtgcccgccaccatgcccagctaattttttttgtattattagtagagatgggatttcaccatgttggccaggctggtctcgaactcctgacctcagatgatctgcctgccccagcctcccaaagtgctgggattacaggcatgagccactgtacttggccatacattcatatcattctcataaccatccccgattcaatcatgatgacagagtctcagaagacactgttatctgggaaatgtgttcattattcttcacctcacaaccctattcttcatcaagtcaatgtggataacctcaaggagggtcaaaacccagaagaatgatgggttggccccttaatcaagggcaagtgtgagattatttcttcaaatctggctgccggtcttagaaaagacctttttgcacttaccagtattatcaaaatcaactcagaaggaaaagctaataaaaactttgctaacatctacttttgatcagttccatcacatggggtataggtctgtgagtatgtttcataaaaagattactcagcaagtcctcttctttgtaatggagagggtttgactaagctgacatattccatcctataattttcagggtaacattttgcaccacgagccaaatcttacacttcggattttaatttcctaccttgcatgctaatgccaaatatcaataccatatttaatcttaactcaaaattcctgtttcttggagttggacttccatcatgagtacaactcaagatcacattcatagaagtgtgcttccgtgtttaaggtacatgacaatgttacccgtttactcttggctttttgttgttgggcattgtagttctgtacacctgcttattggccatcactcatctatctgcttgctgatctcttaattggagatgtattgactcttggttcctctatagctctcaaatgtcacctgagtttggctcttgttctataaaatttctccaaaacaaacatcagtgaactgaaccaccacccaatgtagtatcagatcatcttatagctacaattttataaagatacattaatatcaacatcatctatccccgtcgtctggggcaagatgttatattgtgtcctgtttttttgtgagttttcattcactgcaaaacatttattagactgtagaaaatgtactgacctactttatagccataggggcttaaacttgggaaaggaccagcccaagaataagctcctgatttcctaacaaatgaggcacagtttcccattgtttgcatttgtatttttttaacttttccagttgattaaaaaaccttccaaaatggtaaagtgcctataagatgatatctttggaggaccatacttcctgcagtaattttctttttaaattttcccttgagttatactcactcgtctccaaaatgtgctttgggaaacttgactcaaccagaaagatcctttcaccttttcttcagactctatattgggagcatgagcttcattttttgagaaacttccttccctacctggaagaggggttcttgttggctgtcatgttggttattggtaagggagttcaggtgctgggggacactgatatgatttttttccaacaactctattgaggtctaatttgcatagcagaaaatgctccccttagaagtgattcagtgattttggtaaatttatagagttgtgcagtcattgcaggaatccagttttaaaacatctctcttaccccaagaaatttccttgtgtccatttacagttaatcctccagccccttggcaaccatcactcttattgtctctctaaatttctgtaatcttttaactaaaaagaagggaattttttttatctttctgtattttacctacagaatacttttattgatcatactgtgtttttcttaatgaaattggcccaggcagtattaacacgtgaccctgcatgaacagactgtgtttgctgatggtgataaaggagcatcttgggttccattgtttgttctcttaagtcagaatatttccgttttgtgttgactctaccatctttctcattgaacattaactagaggtaattcagcctcatgtgtgtgtccacttaagttatatctctcgagagaagggacttgaaggttctaatgtctatgaggcttgactacttccatagcggcttgaatgatagcagtcattcttctttgtacatctacaggtacaacttaaagaaaccattgaagatcttcctggtaaaatggatactgaattagcagaatcaggatccaattttagtgttggacaaagacaactggtgtgccttgccagggcaattctcaggaaaaatcagatattgattattgatgaagcgacggcaaatgtggatccaaggtataaagctgaggtccatgtaaccttgagtccacattttaagtgtgtaaatgggaaacatggaatgattctgagaaatatttctaagtgctctgtttgccccagacataacattttaacatggataaattcattctgaaaaaaaaagaagaaacccaaacgtgttttccagtgtcagagatattattcagtatcccaggagagctagattcctaaaatccactcccaataatcagctttaagcaatttggagggaagacatttttcgttgttttatgaaaattagtaaattactacatagatttttatacttgtttttaaagaataagattgtcctaaaacattttaacatgatttttaaaacattaaatagatatgatcttttttttttaaatctctattgtgaagtagagctttttaagtcttaagtctactttttgcatctttacgaaattaaacttaacttctttgattgcagaactgatgagttaatacaaaaaaaaatccgggagaaatttgcccactgcaccgtgctaaccattgcacacagattgaacaccattattgacagcgacaagataatggtgagtccttcacctgcggaattcctgttgctcccaattatgttcactttttcagtttattccttccttgtaaaacttgatagaaacccctagtaagttaattttctgttaattttggctctcttcccagcagtaattcatatgcttaactacaccctcagtgctactaacaatatatccaatgtatctctttgcatataatcatatgcaaaaatcatatgttaaaagtttaccatgaaaatattccaaatctaagattcagcttgtgatgctaactgtcatactacggagtagtatccaatagaagatcacagtcattgtcactgtcacagttgtttcagcatgcttgatccccttaatgaaagatttaggtttttatatactgcaaggggcttgttgggtaaggttgcagaacttctatgctgttgggacgttcacaagatacgtgtcacaggggaagagatgagaagatccatcacaaattattttcagagagggcctagccagaaaaacaaatgattccaacagaaaggatttaacataggaaatttgttacataagaattggagggcttagaaaatgaagggaaacagtgataattttaggaagcaaggccaggcacggtggctcatgcctgtaatctcagcactttaggaggccgaggcaggcagattacttgaggtcaggagttcgagaccagcctggccaacgtggggaaaccctgtctctactaataatacaaagattacctggatgtggtggcaggcgcctgtagtctaaatgtatcatatagacttcttgttgggatttgctgttaccgcattggtgatagctactccttttattatctaatgcttggaagtgctgtcaacttagaaggtctgattagttagagagacagcaagcaaggacagaacttggcagacaagtcagttggtcactcctaaatctgatcaatcagcttctgaccacataaatgtctagttcacaagcatcttcttgatccatcagcgctgttaccattattgaagtgtgcccattagtttattgctttatataccagtccctctaggtagattttgagaaccttaatttttttctctcttgagtaatgtctttatctaaactctgaacatggttctacaaagcaatttacagttggcccctccctacctttctagcctttcctccctctgttctctctctttcctttgcagtaaaagccacagtcctttagttattgtccttcagttcatcaaacgcagcattcactgttgcggtcatgccctctctacaccatgcacaatggcggcttagactatggtgtgagcagtgaagatggtgcaatggatagatttggagtaaatagcaaagtaaagcaactggactgtgatggattgaatgtgagatttgagggaagacaggggtagtcagtcctggtttctctgtttgggacctgactatctggatgaatgaggaagacccggaagaagtaaacttggaggagggaaaggcaagcattcccttggaggatgtaactgttgagatgaccttgcatttcaacatggagctttccttctcactcccaactccattagtgtctccatctatggcgatctctccaatgatattgggagagcttccatttagggctccctggccacgaggtcatcagtggcatcacaggaggtcctaccaggaggtataaatttaagtaaggagtcttattacaagctctgaattctttgtgagaactattttccaatttttcttaacaggcagtggcaggattcagcctcagggaacccaggagacatgcattctgtagcttgagattgggaactggcctctatgttccctcaacccctagctgaaagcatctcctgtattctagagcttctgatcatcagccagctttcaattcgggggaacaccataaataccattcttcctcagatctttgattttggaggtgttttgttttttttgtttgtttgttttttccccttgtttgctttagagataccgttccagaaaacacatgttctgttctgttattctgaaagctgtgaaaacatagagacaagtgaatcagctgcaaagctcagcactccaaaaggcaggctcagggagtggctggggaagtgacagatgttaatgaaggacataaccaacactattgtcatcatgcagtcctgtaattgcaagtgcttaaggtgtaataattacgttttgttgcattggtgaatacttctccacaaagacggatagatatagttttgtttactgtaagtaaatcaaacaaaaagcttgttgggaaacaactgcgacctaattaatagctttgtttatgtgttgactctccaaagggatttacccagttttggttcattaagcgccatcaatgtgttaagcttagaattttattttatgaagatgttctcccagttttaactttattaaacatggtgtagtcttaatgacagctaatttaatcttcatttgctcagttgccacaaaacagtagagcaagacagacaaattagaaatcctcttttttctaatacgcaccactatctccacattaaagacagcttcctccctgtagtttttaggattagctggttacttgtgacctgttttcctttcagccaatagcacagccttttcctccagtccctgcacacccgtccttacaacgctctggattctccctggtagtgtgagtgaacgaggaataagcgaatctgcagttagagaattcttcgggagtgaattacagagaagcaaaaccaggcctgagaaattcagattgtagctcttttctttgtaggcaatcttatgggtgtataatgtatatctttctttctaaacccaaatctctttttagtagcgtttcctcagagaaaacaaactagaaatcgttctagcagaaccagctggagaaacacaactcaaagctaacctgagttttatttaaattctatgtttcatcttcatggaacttctctttaagaaaccgacaacggccacaattagaacatttcagaagaggaaaaattacttcttgaaaaatatgctgagacatttaattaaagtgttcagggggacaaagaactaatgtgaaggtagaaagcacaaactggggctgggcgcggtggctcatgcctgtaatcccagcactttgggaggccgagaagggcagatcacctgagaggtcagaagtttgagaccagcctggccaacatggtgaaaccctgtctctactaaaaatacagaaaaattagctgggcgtggtgccgtgtgcctgtaatcccagctactcgggaggctgaggcaggagacgcttttgaacctgggaggcggaggttgcagtgagccgagatcgcgccactgcactccagcctgggcgacagagtgagatccatctcaaaaaataaaaaatgaacacactgggagaataaatacacacaattgcagagctttggggaagtggctgagtgggtcactgggacctacccttcatcttccccaggtgagaagaatcatttactgtttcaagacctgaggcaggattttaagtaatctttgccacatgccatagtatcttgtcccagagaagagtcacaggcacccattttaccagtgagggaaccaatctccaacgtcagatgactctccgtacacccctttcatcatggtggcccagaaacgacttctgaaatggaaaccctcccaccctcactctgcctgttgctaagtggggtttctctgtattgcttgctgccatagtgaaagctgttccgagcttttcttttcctatggtcaggctttatccttcaccctgccctgcccttcccctgttcctgagatcaccccactatcagcgatggcatcctgccaattaaatggattaaattgttttgcagtttgttcacttaatttttatatctaggacagctacctggaatatctgcagatatttaggcctgataaacacatcttcattgagcttatgtaactgagtaactgactaatgcattccagtataatgtgtttttccttgaccttctagagagcagcaagtgatttgaggtttgcattgctggagagatcctggggccacgggaccaacatcaatgcatggcttagtttacagttttacttgggtagaggaaaggtgaggaccaagcaggagatttcaggactgttttcatgtagctacagaagatatgaagatacaaattcaagattatgcccttttttctgtacaaaaagaaaattcagagtcactttattttgttgtaaaagatctagaatctttggtcaaatcagtaaatacagattgagtcccctaatctgaaatctgaaatgctcctaaatctgaaactttttgagctctgatgtgaagcttgtaggagacactcattggggcatttcagattttggattttcggattaagtatgctgaatctgtaagtatgtataatgcaaacattgcaaaattcaaaagtatctgaaatctgaaacacttctggtcccaggcatttcctaagtatactcaacatgcataaaggctaagtgggtaaagttctattcttagcattttttcatgtgcatcctaacttggcccttgtagcatcagattgttccttattttctcttttccaatatttaatttgtgagtcaagcctctgccctcattcctgctgtgtctcctttttgctttttctctctcttgtctatagccatcaacttgaggcatgtggtcctccaaggctatagcctgaatcctagcatccctatacctccttccatatctgttttccagggccagctagactctttctctgctggatctcactctgtggtcttaccagactgcaccactggtcagtcacccaattgtgccctgaatttcccttcctttgtatttaacttttaggcagtgtgcatggggtgcctgctcacacgtcatgtgcaatgccaggccatggagatgcaacagtgaacaagagcaagcacataaccctgcccttgaggaagtgataggtgagctgtcagacctctcggcatctggaatgaccacggtcctcccacccctcacctccaccaccatttctacgtgtctaagtctgtccattcctcaaggcccagtttgctattttctcctccctgaagctcctcctcagaaattttggccagaggtgacttctgctgctgctaaactcccgcttgttttgccttttttctggcattctagtcacttgagtgttttcttctgtctttttttttttttttttttttgagtatgggcttcctaaaagcaggggctgggacttacccatctttgcctcttccacagcatctagcataatgacatgaatgcagtaagtgtgcaataaatgtgtgctgaataaattcaggaattcatgaaggttcacttccattttcctccaatgtgttttggcagagtgtcacttgctcattattaatcctgaaactttaatgaagtttgttttcttgggccatatgtgggggctcatgcctgtaatcccagcactttgggaagccaaggtgggaggatcgctttgagctcaggagtttaaaaccagcatgggcaacatagcgaaaccccatctctacaaagaaacaaaaattagccaggctttggcggcgtgcacctgtagtcccagctacttgggaggctgaggcttgagtccaggaggtggaggttgcagtgaaccgagaccacaccactgcactccagtctgggtaacagagtgagactctatttcaacaacaacaacaaaaaatttgtcttctttttctgtgagtatatgccaggcacacagtaaataattttgagtcctgggtgtaataggactcaaattgtagtggaaagtgaataggtgaacttttttatgcttacaggcatatttttcttaaaatatgcagttattatccaaatgaaaaggtgtcttagaaaattagctgtttacagttctcattgcccatattttacttctgaaaacaaagaaactgaaagtaaataataatgtacctctgggaactcacatgaagaggaaagaagacaggtttacacccgggttcaaatcccagctctgccatcttccgatttgtgcagcctaggctaagttgtttaacttatttgcacttccatctttatctctggctgtcagcatcttgatagactgtttcttgctcatctttgaatctcccattcctaaaatgatactttggaaatagacattcactcaatattggatgaatggatggaaggaaggaaattaccttatccattctattttgggagttttctgggatgactgtaaaagcagagcatgttcaacccattttattttccttttcaagttcacaaactgatattttagttatggggtgctttcattgatttttatggtagccatactctttgttaggtcatatttatatgccaaggatgccttgtctcaaatcttgcagattcatctgcagctataataatccagttcattataagatactatatttggggggttatttgggattttggggttttttttttttttttttggacacagagtctcactctgttgcccaggctggagtgcagtggcatgatcttggctcactgcatcctccacctcctgggttcaagtgattctcgtgccttagcctcctgggtagctgggactacagttgcacaccaccatgcccagctaatttttgtatttttagtaaaggtggggttttgtcatgttggccaggctggtttcaaactcctggcctcaagtgatccacctgccttggcctcccaaagtgctaggattataggcgtgagccactgaacctggcctatattttgttttaatgcatatcaaaaaaatattaaagaggtcggttttattttatattttttaagtgtgaaaccttatagaagttaaaattttaagttctagtaaattattaaggtcatggttctatcacccctaaactcaaaaactctcctaacaaataaaatgcagttgctaagttgttgatatttataaggcttaaagtattttgaccaaatgatctttctgtcatttttattattaatctaataataataatgctttttgagaacattatttttttttttctggctctgcctttttagattctggtcaattttgagaactcctggtctcagtggcattcatgtgttttcagacaatgagagggaatctggaaagatttactagtcactcaaatgtgaataaatttagtaggaatttggacaaggaccaaatagtagtaggacttcctttccattaaacattatgtaaagtctttacagagaactcactcactctcaactctgtgggaataatttttaaaagtttacttgttgcttttgcagactaaaacttcatcattttagcaagtaatttggttcacttatttacttagtctttagcattagagagacctgtatttttaaattgcagattttttccttcctcgtcaggaggagtggttatgttctgtaatgctatgcttaagtaaaattacacatttggggtatctaaattggcatgccaaattataggacattttaattgttatgtggatgtgacagattcaattattcttgaactcttttgagaaagctgcccttagcagtgattcacagtagtaaacaaaagattaacaggaaaatgccctttatgaggcttaataaatgtgtccgattaaataccatgggacggaattaactgcaatatctacatgtagaaaaacaacctgtgtcctgcttggaggttcagcatattctcttaagataaatttactctaaatatggcctaactgaagaactaagtgccacttaacctcagaactgttttcctaggtacgtcatttgggccaattgttcagactctttcttccctctgaaaccattaaccaaagccactaataagtagaaatgcagagttaagagccccttcctccctctttcaatttccagtggacatattaatgagcaataaagcagctaaaaggcaagcagctcaagaaagagaaagaagcaaagaaaggtgattgggaaaacagatattcagccctcagcaatgcagactgggctgccccgtgtgccatgtgggctgtgatcacaggaatttggagctcaatttcttaattggttctttagtcttatccaaagagaaaattggatttctagagatttcttctgtgcagagagcaattatgctaaagctgcttaggtaacagctttttcggctagcctttctctctcggaataataaagtacacccagagccgatttcagaatgcaccttaacctcaccatccagagtttagcccctcttggcagctgacatgttctagaaccttaaagaaattccagaagcatttacttcttttccattgcttagagtctatgaaactggctcggcaattaatttactgtcaggtctctcccatctgatttttatgacattaccctaccttttagataaatttaatttgactttcttaaatcaagaagtttcccaactctgatcatatgtataaatacccctcaaggctgagagtctgagagttaaaagcactaagtaatgataagaacccaaaatgcctttggcagaatataaaatgccacaggactgctgagtgcccagaataatagtctagaactttcattttcttcctttcctataacattatagtaataaaaggtcaggttaggcctttgtttgcttcgtaaaacagggtaatcaaaccatttaaatactatccaggcctaccacccaaacagattctctccctctcctgcccctttccgaaatattgtctctgttataatatgttactgcaaaacattgtgtttcaatgggtttctttctgctgattaacaggttttagattcaggaagactgaaagaatatgatgagccgtatgttttgctgcaaaataaagagagcctattttacaagatggtgcaacaactgggcaaggcagaagccgctgccctcactgaaacagcaaaacaggtgagcgtgctccaaggagttgtaattaaagtttgtctcagcggctccctccgcacaatttggcaccatgggtatggtaacaagcactgtgtacccttttcaattataagcctgcagagacaaagcttcatgagtgggtcttaacggcacgtcccgggttgaagtaaggtctgcaaagagttggcagaagcgggtaggggttggggggtcgagtgtgtgcacacatgtgtgcatgccgtgtgcccaagaagcggagaggtgttagaccagactttttacacatttagactctgtgggttcagattctcatagaaaaaggcatagagcacatagtactcatattctgcaaacccattgcatttcattcttattgataggcaaatgctgttgaggtacatacaggagatttaagagtttgggcaaaaagtgtgggatattgtttattgttctagaatgatctacctagaaaatgttctacctagatctacctagattgcttttggtttctgttttattatttgtaaagaaaactgttacataatactctcactgttgacctgggttttcccctcattctttgttgtttagaattaggcccatatagcccatatgcttcctccaccttctataagaggcagttgtcaggctgggcgtggtggttcatgcctgtaatcccagcactttgggaggccaagcaggcagatcacctgaggttgggagtctgagaccagcctgaccaacacggagaaaccccgtctctactaaaaatacaaaaattagctgggagtggtagcacatgcctgtaaccccagctactcaggaggctaggcagaagaatcacttgaaccgaggaggcggaggttgcggtgggccaagatcacgccactgcactccagcctgggcagtaagagcgaaactccgtctcaaaaaaaaaaaaaaaaagatgtaattgccatcaggacaaatcaagaatttataatatgttatagctatagcctgaataatccttgaagtatgtttataaggcattaaagacacgcaggctacgtgatctctttccaacaatggtgatgtggaagtatctcattctacagatgtgataactgcagtttgatttaagggacttagccagatgtcacatggtcacctgtaattcttccttaaaattctgtgaggtgagagttgtattgatggagggagggaaggaacatccaatgttcttggcttagggtggaaggagacttctagcagtacatggtgaggctcccatcaaaactgttcatccataggcagtataatatgggtataattttccttcattgttttctgtatttttcaggttttcatcactgtaggtagaataaaattgcttgtttgtgtgtaatattttaaatatctattacagacaacagaataagaaaataggggccaacatggtgaaaccccatttctactaaaaatataaaaatcagctgggtgaagtggcgattgcctgtaatcccagctactcgggagactgaggcaggaaaatcacttgaacctgggaggtggaggttgcagtgagccaagatcatgacactgcactccagcctgggcgacagagcgagacaccatctcaaaaaaaaaaaaaaaaaaaaaagaccaggcgtgatggctcacacctgtaaccccagcactttgtttgggaggccgaggcgggtggatcacaaggtcaagagataaagaccatcctggccaacatggtgaaatccatctctactaaaaatacaaaaattagctgggcgtggtggcacacatctgtagtcccagctactcagggagactgaggcaggagaatagcttgaacctgggaggcggaggttgcagtgagctgagatggcgccactgcactccagcctggcaacagagcgaaactgcgtctcaaaaaaaaaaaagaaaaaagaaaaagaaaatatgttcccatatctaatggtatttctgtattaaagatttatccaaataaaacctaggttgccttcagctcctatttcatctcattctccacagaagtgaggaactatccttgccactgtctgtaggatatttctttgaatcctgcatttatcctcaaaacgattctataaatgcctttgtgtgcacagtattacgctgggcactaacaaatacattaaacatgcccctagtcattgacaatataagccaaagaatagagcagagcagagatttgattgatgtggtgagccctgatggcaatgatgatgatgatgatatgtgtcagatgtgcactgtgcacttctcattatgcctgtttaagaggctgaggcatagagagggaaggaaattgcccatgatctcatagctagcagaattataggcagagtccggattcaagcctggctttaggatcgacaaaaatctgtgttctgaattttcataagaatcacattgtgtatgaataaagagggaagctgtacttgaagcactgtatttcttttatacttgaaatttacagaatacttttatcttctagcgtgtctcccagccaaagcttccctcataaaacccgatcaatcatttgggattccctgtataatctagtcacaaaagagccaggaacctcaaaaaccctttttgttctttggtacctcaggcatttcacatcaaagagatgtgtccctatctatatatcaaaatacctatttaaaaaatatgttatatattatcaaggcatggagtaataagaaataggtcatagaattttagcatgttccaactaacataagataatatagttcacctttgttttacagatgaggaaactaaggccccaagaagtaagtgggccctgactaatttatagcagagacagaattaggaatctttttttaaatcaatgtttaatgcaagtataacatacatatagaaagggacattggtcatagaattgaaatctcaattactggtccagtaatcttccatctaattcaacaatattaatcgtaacttacgttcgtatagttctttataatttattttacattgtttacatagttcttacatgtatgattctacttaatcctcacagcagtctgtaagtcttactgtattgatgaggatgttgggtcatgtgtcttcatgtggacacagagctggtccgtggagaacagaaggtagattgctgggggaggccgatgtcttcccctaagtagcgttctgtgtcaagcctgaaaacgctgctccttgtctcatttggtatccaagatgcagctagtcgcccaaattcacacagtggcagaatttccttcctggattttagtcctgcctaaccactggctcaggtcacctcctctacagaaagacataaggaaatacacagcttgattctcatccaagttgaatattttcttgacatttctgcatacacttggtcctcttaggagaacaagctgtcagattgtcaaccttttaaatgtgcaagcatcaaatatcaatgcatcattcacaactgtaacaccatgaggattagtctaaatcaacaagcttttcgctctcacagattagaattcatttgttggaaggaaaactcaacatctgggaaaaggtacttccaagtcacgtattcttgaccacggagttgctttcctgaaaatgctggtgcaggcaaagctgagtctctcttttgactgacctttgcagatgactcagtattttaatattttaaagtaaatttaaattgcataattacatttcaggtacataaagctgaagggtgcactggtttgctcaggctgccatagcaaagtagcacagactagctggcttaacccacagcagattatgttctcacacttccgtgctggagtctgagatcagggtgtcagcagagctgctttcttctgaggcctctctccttggcttgcagatggctatatgttcacggggtctgttcacggggtcttttctctgtgtatgtctgtgtcctgatcacctctttctctaaggacaccagtcatattggatcagggcccaccctaaaggcctcattttaacttaattacacctttaaagactttctccaaatacagtcacattctgatgtacttgggggttaggacttcaacataggaatttagggggatgcaaatcagcccataacacagggtaaattcaatcattgtcttccctgatgaagccgacctaatttgaggttcccagtgagtaacagaggtgccattttatgcactcatgaaattgccagatacaactcaaaagttatgaaatcctggccaggtgtggtggttcacacctgtaatcccagcactttaggaagttgaggcgggtatatcacttgaagtcaggagttcgagaccagcctggccaacatggtgaaaccctgtctctactaaaaataaaaaaattagccaggcatggtggcgcatgcttgtaatctcagctactcgggaggctgaggctggagaatcacttgaacccaggagatggaggttgcagtgagcctacgtcacacgactgcactctagcctgggcggcagagcaagactccgtctcaaaaaaatatggatggatggatggatggatggatggatggatggatggatagatagacaagaagtcctgacatcctggatcctaggagcctggggcctgtttgaataggtggaggagacacttagcagccaatggccttacagaagtcacccaaggcctgtgagcctcagaggcctcacctgtcatgccaggattgcagtactgctccggtttcttcactgttaacatgtattaaatgagatagtgtatgtttaagtgagtcaaagaatataaggtgttttacaaatataagcgaattctagctattggttataaaagctcagtacttttttaagactgtagactgaactcttagctcccaataatagcaaaagaagtatccaaagttttgtagctttatagactaaataattacaaattatttttaagaatcagcttaccaaatgatatatgctcatgttacaaaattcaaagcattgatagttttgcaaaaaactttaataagtaaaaagtaagtcactctgccacacctgcccttcatcacgcagttcctctgccaggtttcagccctggcaccttcttctccactttgttgatgtattctgtgcataaacaagcatctgtgcatatatagcttttctggtttttgagcacaaataatagctattctttacggtttttcacttaatattatattccatattatatcaatacatatagttacctcattttattttatatgtgtattttttattagcgttctctctctctctctctctctctttctctctctctctctctctctctctctctctctctctctctatatatatatatatatatatatatatatatatatatatatatatatgtattttttttaaagcagtttcactcctgttgcccaggctggacctctatggtgcaatctcagctcactgcagtctccatcaagtgattctcctgcctcagcctcctgagtagctgggattacaggcgcccaccaccatgcctggctaatttttttgtatttttagtagagatgggtttttaccatgttggccaggctggtcttgaacttctgacctcaggtaacccacccaccttggcctcccaaagtgctgggattacaggcgtgagccaccatgaccggccagttacctcattttttaatggctacctagtatttatactttttccactctgtacttttcacttttcccaattaacttccaattaagttagctatttttgtttttttcctggtgaatgacttaccagatttatttactagtttatattctgttttccagttgatcagtttctgcttttatctttattacctttgttttttggtttttctcgttgcttcttcttgaatgcatttgtgtccattctttcttccttagtattttagcgctgtgaattttcgtattctgttggttttgatatgtagtgctttcactcatagtttattttttatttatacattctgcaattttggattttaattttctctggcccagaaattattgaagagggaagtttttaaaactttttggtggtagcggtttgttttttctgttcatttcttttctgttttacgcatcagtttcattttcattgcatgtcatccatatcctttctatccatttggaattttctttgtgtcctagtttttctagatgttctttgggcattgaaaagatggtgttatctgtttaaggtatagagtttaataaggtttaattagttctactttgttaattacattcttattaatccttatttatattggtctattttctgtgacttggatctagaggtgaattagtttcacactgtacatatttctctttgtagtacctgtcatttttgttttggtaactttgatgctatggttttcagtccacagatgttcataacttgggtttccactatgaattatatttcctgccattttaacacacctttctttgattcgtgtaatggttttggtcctaaatccacactggttcaaaactagactgggtcgcgtgcttttactttgctttctttgtctgctatatatgggattgtctttgaatatccaaaggtgtgtctcctgtatatggcgtggttaggttttactctgaggtttaatcttagcctttgtttttaatagatgattttagtacatttacatttattgagattataagtatgttttgtcttagttctgtcatgttatgctttcagtttttagtattcctttttaccttttaatttgtagtagataatttttgtttctttgaatattgttttgcatgtgttccttttgtttcctctaaatgctgtttcttgactccccattataaacaataacaatataaatacacttctgtctccatcctaccaccaaattttagcttagtttttcttctatttgccttcaaaccccaaatatacttaagcctctgttatttggcttttctttaaatgatactttctccatcttttagttagctaaagtttatccttcagtgctttttaaacctcaggctacaacccactagtggtgctgtggaatcaatttggtccatcatgactagtgtgttttaagatgtaaaatagaatacaatggaatagaagaaaaataacatgtatctttcatagtaatggtaatattttttgaaatgcttttcagtatagatacagatataggtagtggatgcgtaccaagtccctgtgtacagtgaaatgtgccacccattattgtgggtggcagtttaaaaaaaaaaagttgcaaaacaatgctctataccatctcaaagggaaaggttccttggaatttagaatccctgacttattgcatgtctgaaatctttgttacccctctgatttaaatgtggtggctgtaccatggcacacatttacctatgtgacaaacctgcacgttctgcacatgttatcctggaatttaacataaaaataaaaattttaaaaagttgtggatataaaattattgggtcacaccttctttcacttagtatgttgcagcctttgctctaaggtcttctcgtgttgtctgttcatatggagacatctatgaccatgtttgacataactcagccttttttctttctttctttctttttcttttttttttttgagacagggtcttgctctgttgtccaggctggagtgcagtggcacaatcatagctcactggagccttaacctcccaggctcaagcaatgctcccttctcagcctcccttaaagctggaactagaggcacatgccaccatacctggctaattttttttccccccatagagacaaggtctcactgtgttttccatgctggtcttgaactcctgggcttaaaccatcatcccaccttggcctcccaaagtgctgggattacagatgtgtgccactatgcccagccagatcttttttcttgtatgctgaaagggtcattagttttcctaggacattattgtgcagtctttcagactttgattcaagttttcttttttttctggaagtttacttgaattatatccttgaatttttttatttgattatattctcttctttacaaatatctatcatgtatatgttttatgtcctttgtctattttgtaaagctattttctccttaatcctttttagttctttattaacttttagttattttgtttgtttgctcaatcctatcctgtgtctcacactgttttttcagcattggctaatctttgtgctgccttcagtagaaatgccacttatttttttattttttattttcccttttttgagacagagtctccgtctattccacaaactggagtgcagtggcgtgctcttggctcactgcaccctccaactcccggttcaagcaattctcatgcttcagcctcccaagtagctgggattacaggtgtgcgccatcacaattggctaattcttgtagttttacaagatttcattatgttcattatatgatacaagttttcattatgttggccaggctggcctcgaactcctggccctcaagtgatcctcccacctcagcctcccaaagtgctgggattacaggtgtgagccattgcacctagcccatttctttgatattttcacttttcttttccacttcacttttgaccactgtcaactcatgtttcagcatcttctgttgctctgccatgttttcctgtatttttgtatctctgttatgcacagcctgatcattctgctgcattttctttgaatgaattatattcataaactctatgtgctcaacatttgtcatgtgtttatcttacttcatttcttatttgtataatctccaggccctgtactactcttaaaattataataataatttttattattcctactcctcacctatgactaaaacgaactcttcctagaccagatagttacaggatagttgtggctagggccttgtttctggctaaagggaattttctgtgcttaatgtagaagtactttatgacccagagaggctgcgtgtgtggtgggggggatgcagggagggatgtgagtgtgtatgtgtatgggtgtgtgggtgtgtgggtgtgtgtgtgggtgtcaggggatgtgggtgtgtatgggtgtgtggtgggggtatgtgtgtgggggtgggagggtgtgtgtggggtgtgggtgtgtgtgggggtgtgggtgtgtgtatgggtgtgtgggtgtgggtgtgtgtggggggtgggcgtatgtgggtgtgtgtgggggtgtgtgtgggggtgtgggggtgtgtgtgtgtgtgggtgtgtgtgtatgggtgtgtgtgggggtgtgtggggggtgtgggggtgtgtggtgtgggtgtgtgtgggtgtgtgtgtgtatgtgaaattgagagtgcatgttttgcctttcttcctccttagcttacagaattgggcaattgcaatctgtcttctgtcaggacctcaccaatgtacataggtcagtcaaagatgtttctaattgattctttattgacataccttagcgctcctccaattaactggagaattcctatgattgttgaggcttgctcttcatagacatcttctcagccttgccacagccccattggttccagttgcatatggcagcccttcatgatattttaccattcaggctctcctgatttcacaaagatttagcttattttttattaatgaatatctttgatgttcagagtggtttctgaaaggaaaaggagaggaaattcattactccattatattaaaagcagaagtcctttctaagtctccattactctttgacaatatgcatgccaattatgtacaccaaaattagactggctttaaaaatgatgaacttgaggccttgagagttactgtgattgaattagggccatacacatcagtaaaaatgaactgacagggtcttgagcctgaattcaacacatcagatggtatcttaggaatgcaatgtcaatcaaagcccagttgctctcctttgtccacgtgcgggagcttgtattgctctgttgattttagtaaaatctagctctgttacggtggctttactgatgagttagagtttaaatatgaacttagtttcaacctacattagaggaactataatggagcaaactcaagagaagaaggaaaagagggacaaggtttcccatactcactgacctgatactcttcttgccgcaaacgtgaacgcttatggcattgtctcccttaagcttcacagtccttctgaactgctgttattatccccttttgacacatgaaaatgctaaagtacaagaagctgagtaacttttttttttttttttttttttgagacagtctctcactctgtcacccaggctggagtgtagtgttgcagtctcggctcactgcaatctctgcctcctgggttcaagtgattctcctgcctcagcctcccgagtagctgggactacaagcgtgcaccaccatgcctggctaatttttgtatttttcgtagagatggggtttcaccatgttggccaggctggtctcaaactcctgacctcaagtgatccacctgccttggcctcccaaactgctgagattacaggtgtgagccaccacgcccagcctgagtaagttttcaaagccaccagttcaaaagtgttcatactccatcttctatgatctttcagttctacctttctgcctcccaggttaccatattcattcagttgaaattgtctcagccacagctttgggattctgtaaagataaaagatgcaccagagggattcctaaatgctttgtcttcatccttcgagctgggcgatacctccacgttctgtgtgcttaggggccacttctctgagcagcattcatcatgacacccaacactaatgcagcaggttttcaacctgtagtttgtagccattaatcagttcctgtgacgtaagctattgttatctcatgttacagatgaggcagctggtagagagagtggttaggtaatttttccagaatcagcagtgaatccaggagagagctgagactggacctgtctatcccagacatagcttacaacatgagggctttgctaaaagtagttcagtaatctcaactcccttcatcacctgcactgtaaaaaagatgtttcatctcacctgtgctgttacccatagtgtgttataaaacctggtagcagaagagaaaaagaaaggtcaaatcaaagaatatatacacaggaagtttgtttttgactaagtatttcagaccatgaagctaagaccatttgggtttaaagttgctaataacaacactttctttatatatatttaaaaaaatacatcaagagggttacacagaaggaagcagcctatttggggaaggctatcaagcagtagaaagtgatgtgaatgtctatttgttcttttggctgtttttagtttcactacagaatagcctttttggagtacataaatctataaccacacaaaatatggctagcttaaaaaagtgggagagggagatgatatgaaattatcccctgaaaagtcttacttttgaacacttgcattataatggaaatagcctcagatttcttctggtggcattccagcattattggacaatttattttctccttaagtgacattttagtacattgtgtgttatatgtaatgatatttctggagtaacaagtgtgcaagaagtgagagaaaaagaaagtacacttaatatgtaggcaggtgtctcagcactttcttttttgtacgtgaatcccattatctccagtgataactttgttgacagcacccaccaatatattcagagcaaggttttataaaatagatccagaatcgtatgactaagagacccaggtgacacgtgtcagcctcagatagttgctacattttctgttttaaagattcaaacaaaacttttcacattcttctttggggaaaaaaaattacatttgaagtcctttttcacccaaacacgttttgagataagagctcacaagtcttgacattatttcattttgttatgcgcatctgtcaaggaaccaagatacgatacagctactaaaaactgatgattgtggaacagagaaacttgacagcatttcatttcctttatgttctgattatacgtttatcagaaaacctgtatctcttctagttcagctagaaattggtagaattcaggaactcaaaggcttggcaaagaactgtcctattatttggtatcatcttacattttagagaagttgcttggttgagtcattgttaaagcttttgcttctcttttcattattgtgggccactatccacagaccacatttctataggccctataactgctttgccgcacaaattaaacgccgggatgtttcatacgtatatgccagcatttcaatgcttgtcaatcagagtttataaaatatagggattacaacttgatattaacatccaaatctattgtgctcattttacgtgtgaaaatgagaaaccctcatgcagacgctagatttcccagcctctttgtcaccctgtttttaatttatctccaggttttgagtcagtaaaatgattccgcttatcaatttaatacagagataaaacatatctatgaatcagagtgatcggaactaaaatggatctacaggttgtttggcattcagcccagccctgctgagggttttagtggggctaatttctctgtacagtagattcatccctgttcttaaattgtgcctcagtgagggatatgatttgcttcacatgaagaagtagacacagaagtgcattgtttcagggaggagactttaaaattttgaaacattctttttatgcttaccttttttttttctaggtatacttcaaaagaaattatccacatattggtcacactgaccacatggttacaaacacttccaatggacagccctcgaccttaactattttcgagacagcactgtgaatccaaccaaaatgtcaagtccgttccgaaggcattttccactagtttttggactatgtaaaccacattgtacttttttttactttggcaacaaatatttatacatacaagatgctagttcatttgaatatttctcccaacttatccaaggatctccagctctaacaaaatggtttatttttatttaaatgtcaatagttgttttttaaaatccaaatcagaggtgcaggccaccagttaaatgccgtctatcaggttttgtgccttaagagactacagagtcaaagctcatttttaaaggagtaggacaaagttgtcacaggtttttgttgttgtttttattgcccccaaaattacatgttaatttccatttatatcagggattctatttacttgaagactgtgaagttgccattttgtctcattgttttctttgacataactaggatccattatttcccctgaaggcttcttgttagaaaatagtacagttacaaccaataggaacaacaaaaagaaaaagtttgtgacattgtagtagggagtgtgtaccccttactccccatcaaaaaaaaaaatggatacatggttaaaggatagaagggcaatattttatcatatgttctaaaagagaaggaagagaaaatactactttctcaaaatggaagcccttaaaggtgctttgatactgaaggacacaaatgtgaccgtccatcctcctttagagttgcatgacttggacacggtaactgttgcagttttagactcagcattgtgacacttcccaagaaggccaaacctctaaccgacattcctgaaatacgtggcattattcttttttggatttctcatttatggaaggctaaccctctgttgactgtaagccttttggtttgggctgtattgaaatcctttctaaattgcatgaataggctctgctaacgtgatgagacaaactgaaaattattgcaagcattgactataattatgcagtacgttctcaggatgcatccaggggttcattttcatgagcctgtccaggttagtttactcctgaccactaatagcattgtcatttgggctttctgttgaatgaatcaacaaaccacaatacttcctgggaccttttgtactttatttgaactatgagtctttaatttttcctgatgatggtggctgtaatatgttgagttcagtttactaaaggttttactattatggtttgaagtggagtctcatgacctctcagaataaggtgtcacctccctgaaattgcatatatgtatatagacatgcacacgtgtgcatttgtttgtatacatatatttgtccttcgtatagcaagttttttgctcatcagcagagagcaacagatgttttattgagtgaagccttaaaaagcacacaccacacacagctaactgccaaaatacattgaccgtagtagctgttcaactcctagtacttagaaatacacgtatggttaatgttcagtccaacaaaccacacacagtaaatgtttattaatagtcatggttcgtattttaggtgactgaaattgcaacagtgatcataatgaggtttgttaaaacgatagctatattcaaaatgtctatatgtttatttggacttttgaggttaaagacagtcatataaacgtcctgtttctgttttaatgttatcatagaattttttaatgaaactaaattcaattgaaataaatgatagttttcatctccattcaga

SEQ ID NO: 2 - DNA, 1708 bp, Homo sapiens
cattcccagcctcacatcactcacaccttgcatttcacccctgcatcccagtcgccctgcagcctcacacagatcctgcacacacccagacagctggcgctcacacattcaccgttggcctgcctctgttcaccctccatggccctgctactggccctcagcctgctggttctctggacttccccaggtaaggctgaagggagagtcagggtcagaggcagtggggggctggcagtgggtaatgtctagatctcagattgcttggcactggacgtaagggacatggtgagttcaggcaaatgcttaggtctgtatatttcggtgagcctggcaggtccctgcatgtgtgcctgtaagtgtgggtgtccctgtgcccctgaatcccaatgcgggtgtgtcttttagcacatcctggggcctatggctgtgactgtctctgcaagctctatttcctgctgcctccgtttcccaggcagaaggagatttgcttaaggtagggagaatggcagacactaagccaaggctgatattcaaaaattttagcaattaataaggctcctaacaccagcctaccctaccccacgcccctcccacactgtgacctgcattaactctttacttgctggattgtcctgggagagaggttggggtgatcagggcagcaggaagtcatttgtggggctcagggcagtggttatttacaaaacagtttagaagtatgacaatatatttagcaaccagtagagccagagccatattggtgcataatggctgaataccaggtctaactcctccccgaccccaccagacacacacacacacacacacacacacacacacacacacacacacaggtgtcctcaatccgagctagggccatggccatgccaccctcaagggtccctgtgtcctgtcaccttctttccccatcccagccccaactctgagtggcaccaatgatgctgaagactgctgcctgtctgtgacccagaaacccatccctgggtacatcgtgaggaacttccactaccttctcatcaaggatggctgcagggtgcctgctgtagtgtgagttgtggggagggtgtctagcctcatctcccgttttagcccctgttgagactcccaccttcttattccccttctccatgccaagcccaaccccttcaaggaaatccctgaaacaggttcccagacatggtccccaggcatgctggattctggggctccattctctccggcctctcaccccaggttcaccacactgaggggccgccagctctgtgcacccccagaccagccctgggtagaacgcatcatccagagactgcagaggacctcagccaaggcaagcctggccctccctggccctgtctcctccctctgagctcctgtctaagattccagcccatgatccttcctctccttcctcctctagatgaagcgccgcagcagttaacctatgaccgtgcagagggagcccggagtccgagtcaagcattgtgaattattacctaacctggggaaccgaggaccagaaggaaggaccaggcttccagctcctctgcaccagacctgaccagccaggacagggcctggggtgtgtgtgagtgtgagtgtgagcgagagggtgagtgtggtcagagtaaagctgctccacccccagattgcaatgctaccaataaagccgcctggtgtttacaactaa

SEQ ID NO: 3 - DNA, 83528 bp, Homo sapiens
ggcggagagaataaagcagaaagacaggaagaggaggtggagttctacagttagtgtggttttagtttttcctaagaagtggcgtggtttggggctttatatccgggaggagcatatgtacgcaaatcctggggcgtttgcaaacccggatccggggcgtctggccccatgcccggccgggcgtttgagggctactgccacgcagcgtttctggagcctgccggctggtgccctggtggcctttatctctgtccccctttgtcctctttatctcaggctctccaggaggccggggggcccactccgcctatcgctcccctcggctacgctgccacttcaatgccccgcaggtcgcgagctgctgttctttcgaaggcgtcggagaaccaggggcgtcccgcgccacctctgactcggagcagcgccgagcactgacgctcccgcccttgggcaaggacgccagtgcgcccgcgcgcgtccctctgcgcggcagcccgtcgcgggccctcaaggggaagcccaggccaggatggccccgggtcgcgcggtggccgggctcctgttgctggcggccgccggcctcggaggagtggcggaggggccagggctagccttcagcgaggatgtgctgagcgtgttcggcgcgaatctgagcctgtcggcggcgcagctccagcacttgctggagcagatgggagccgcctcccgcgtgggcgtcccggagcctggccagctgcacttcaaccaggtaaggccgccaggtccgggcagccctccgtactgtgggtgtccctagacttcactttgtatatgagccgtttttatttgggaaaagtgcaggagaaagaatcttgttatatttctcaagtagaaggcggtatctggtgacacttgcttttcactagacgaggctgaaggattaaaaggttaacgttgttaagggatttggcagccggaaactgcagttttacgcctgatgaactgagaggtccgactctggtgattttaacattttatagaaaactttatgaagtagttgagggtgtgacgccactttaacagatgctttttagataggaatgagttacttaaatcttgccagaaagataacggctcgcacacttcctacaaggttagcaaggaagattatcacatgccaccctggttaacagttgcccggtgcgtaaccagtgctatttgaaaacgagatataatgtagttctggctcctgtgtacattacatgaggcattcagggctgaaacaggggcggccttgagactaagattctaatttggtcatccagatggtggtagactaggcttatattgttagttccttgtcatatgtcaggtcaaatatttgattctagtatttatccaaaatgattaaaaaaattttttcagtaataaacagggtactactaggcatttggcaataccttgggtttaagaacaagcttcaaactacgtaggagcagttaaacatcatacctctttgtaaacataaaatcaaattattcctaattatttctataataaatactctgtgattgggtagttcttaaaattgttaagtagacaaagtgtagtttctgcatccttccaaaactgctctccgtcttttaaaactcagttaactcagtgctttgacttgttttcctttactcctgtcccactgacagctgatattggagggtagaatacggtattttccatatcgtttgtcatttttggacgatgttagtttgttacaagaagtttttggggtccaagatgtttgggaaatgctgcatattatactctgctcttggaaatttggcgtttcctaatgaatatgactttagattacagttagggaaactgtaatcaaataaattccatgttaataacattatacgtagattgatctttttgttagaaatatcttaccacaattaaagtatatggctcttaagaaaagtagaactttctgatgccatggcatgtcagaggtggtctctctttgatctagaagggttttgtggtccccttatgcatgttctttagtaaattgtctataattccagttctgggcatggtattttatctggggtaatatctgagaaatgatgtgttggaaaaatggaaatgcccacaaattatgctaatatttaagaaaataaagtgtttgctggctagttgcattcttttcttatcaaataggggaaacaactgagctgactccagtatttccaggtaggtatggtcacgtgcttggaaagaagtttagaggattgacttgaagctgcatatctgtagcccacacattgttttgacttgcatgacctccttatcttgcagtgtctggaggtgttgatgaattttaaaggggcaaaagacctccccacatcacctttgcaaagacattggaaaacatgcctaaattatttaggaaaattggttaagtgtaccagtaatacgattctctgtttattagcaggcaggatgtctcaataggtaaaattatatgtatcttgacacctgttttctcattctcatgtttgcatatttaactgccctcattatggcctctgtgggcatcaaaaagagacaggaagaagcccataattaccttggaaagaaaaggaagccaaggaaaatttaccacttaataagcctaacaaacatctctttgatgttcttgctttttttttgagactgagttttactcttgttgctcaggctggagtgcaatggcacgatctcagttcaccacaacctccccctctgggttcaagcaattctcctgcctcagccttccaggtagctgggattacaggcgtgtgccaccacaccgggctaatttgtatttttagtagagatgaggtttctccctgttggtcagtctggtctcaaactcccgacctcaggtgatccgccggcctcggtctcccaaagtgctgggattacaggcatgagccacggtaccttgcctgatgttctaaaaagtcctaatgatgacaataatcattggttaaatgatgacaaatgaaataccaatctaaaagaatccattgatttgctggtaagtgatggacttgagcccacaacattcatgtatgctccagtgtgaaaccacccaattctggagtaaaattttagggtcattaatgcttacataccctgaggaatacaacttcagtttcctcacagggtggaataaacaatgcacatgtctattgtgagacactggtttttggctttgaaaaaggctaagaagataaaatttgtttcaaggtccaggaatttattgaatcttctacaagatttagttttcaaataacattgttcttttggttgagggctgtgatatttattaggtcagcttttctcaggtaatagactaatgaactctggagatttgttagacaattttggaaggttttctaaatatttttcttcgtatccttaagtaaggactactaaattagcttgttgctttgggatatcaaagaggtcagagttcaaactgtagacagtagaacaattgagccaatagttttgcttcttatagctactgacagtgcgaagagttaggtagaagcagtgaccaaaacaacaaagaagagggagcccttttagccaaataggtctctggggccctcatttctggataaaggtctgatctattgaggggagttcctagaatacacagggaagccccaaatatctggatacccctgggttattaatctaggacatttaagaatggcctcagtgctgccttttgtttgttcagctgttcctttctcattcttcaaatgttccttttcctcatttcctctttcaaacactgtattttcaagttcttatgtgtagatagtggttaaacagcctgataaaaggaagatcctattttctaaattatcacactgagctgctggaaatgcctccattcagaatttcttcttctgatatatattttttgatatatatcttgcagcccatctcctgtagtgcacatagtttttttcttctcacatttctactggtaaccaagttctgtttttaacctttttcagttcatattcagtcttctcaaagcaacactctttcaaaaagcctccatgaactgtctcagctggcaaactctaaatcttgagcctagcatttaaggcctgatcctcctgtgtcaacctacctctccagacctagcacccactaaactaaactactcactgctctatggattcccataagcttttgctcaaccacttcttaactcattatcttccttggacctttcctgcctttgactgaaatgttcttcttttccttgaattgccctgtgaaaatctactctcttaagcccagttaaacttcaccataaaacctttctggggtatcacagccagcactgatttactgatttctctattttctgaattcctgaaatattttttaccatgctgatgtcacttctattcctgtgttcagcagacattttaaatgactgctgcactctaatttgaatggttatttaaaaatacaaatctgatcatatcattcatcctgttaaacaattcagtgacttttagataaactacaaacacttagagtggcttcaaagtccttttctgatttggtccctgtatgtttcttaaaccgtatcctttgtcacagttccaatcttaatggaccatttacactgcctggttctctagaaatacctgattatgctctggtatcagtttagacattacttcttctgagaagcctttcttgatcttctcaagagtgtagtcgaagtcccttcaatgcaatcctgtatttcttggtgcttgtgcaattataacatattatatattgcaattatattataacatgtaaattattttatatgtattacagttttctgcttatgtggctataacccctgctgcctctctctaccctccaaaagatttttaacaagttgaagggcatggtctgactcctctctctagagactagtgtagtgcctggtttataacatgtattcattataatttttttgaatagatgggtgaagatggtttaagatgagtttgataatttgagtcctcacatgactttacagtctattggaggagacatgacatatgcacatataattataatgcacgaaacagtgatatgcctgattaaggaactaacccatttatgcctgaggttgcaattttttgaatttttgcaatcagaccttggcaatgaccttgagcagtgggatataaataactcccacatgcttagtgtttcaataatggaacactaggaataaatgtgtttaaaaaccaatgtgagataagacctgtgttttcagaacattaatctgacatgagagtgtggaatgtattggagtgggtcaaggtgggagctaaggagccatctgcagtggatttcatgagatgtaaggaccacttgaatttggagaaactcagaaggtaatataaaggctctgccttcagagacttcaagaagagcaggcatggctgaagagtatgcaaaaggccccaaagataagccttttctatttcaatattttgcctgtaatcattatttatttaagtattgctagatctcttccttcattatcttctgaccctgtgatgccagaaggagaaagaataactccgggctcatattaatggatggccaggctagtggtatgtagcaaaaaggagtgtatgtatgtgtgtggatactgcaaacatgttttttgcattgcctgttgtcatctactgcactttttcctggggtgggggtaatgatgggttagatgagggtgtttctgggaagcactgactatggacagaagagataaagaacatttgttcttttttttttttcccctttatgtgctgagaaatggtgggggtaaaccttctaggaatgccttctaacctgcttgttcaggtgagaaactggaagggcaggacagagggtgaggttacctaggtctcttgttaagaatcctccaagacaatggccccattctgatctattagatcactctcacataagttgattgtgtacagtgtgttgattgtatttcaaggctttgggaaaatgagggacattcgtaattcatttgactgtgatgtcaagagcttagtttgccttgatgccaggtgtgattgggatccattaggaaaatgcttttcattcctttcatatctaactcccaaaatcacatctttacccactattctaggagtcatatgaacccttttatatgtaatgtaataattatagctaggatttattaatcttttactgtgtggcattactcccttaaccaatttatttgtattatcatgtttcatactcataccaactctatgataaaggtcccattattatcttcattttatgtgtaagaaaaatgtggcacagagaggttaagtaactggaccaacgtcacgaagtaagtggtagaacctcaggcagtcttacacactatgctatagtgcattttttgaattacaatctagaaaaccatactttctctagctagagcaaccatgaccttagattttgaactacagggagaagttactagtcatcgactgcctatctggttacattgcttagtcacattaaggaaatggtactagagctgtggaggctacgatgatgatgacttgtctttgtcctcagagaccttataattaagaaataagatggaggctgggcgcggtggctaacgcctgtaatcccagcactttgggaggctgaggggggcagatcacaaggtcaggagatcgagaccatcctggttaacatggtgaaaccccatctctactaaaaatacaaaaaattagccgggagtagtggtgggtgcctgtagtcccagctactcaggaggctgaggcaggagaatggcgtgaacccgggaggcagagcttgcagtgagccgagatggcgccactgcactccagcctgggtgacaaagtaagactccgtctcgaaaaaaaaaaaaaaaaagaaataagatggaaactgtgaaaagtgactatggaaggcaaagctgcagtaatcacgaaagcagacattgtaaaacatatcacaaggccatatatgattgtcacatggatactagaaaacatctgctactagtttagaaaggtgagggattttttggggttggaaatgcctcatggaaaagttataactcagtcaatttggcttatctggtttggtttttaaaaaatttattgattgaactcttgcttataccaaatgtgatgaattttcatgtgaattacatgaattcttttttagaggatgaaattttcttttattgtttcatatataatgaatttttttaagggactatattttgagcaacaatagtactttcttctatggatagacttcaggcatgtctagtatgaaacctttcagttcatttctcacttttatgctagaagttttcggccaatgaatccactgtctcttctggcggtttagaatggaaatcttcccttatgatgcctctgaagcaattgcattcttaactgcacaacactggagaaagtggtttgtgcataggtgttggggaatgagttgaagcaaggagggaagagtaggtgagccgagccaagtacttcaacagaatcactgactcttaactataggtaagacagtgctgctaggaggcagtttgagctttctcacacagttatcaggtgaactttccataattgggatagatctttctctctagggatgttccctagggttgttctctcatctgggtgggagatttgcagctgttatgctttccatgatttattcttatcttgggatatattgaggggcagggacttagttttctgctttctagtcataaaaacctattacttttcaaccataataattgtttaaaggtgttttctttaaatttaatatacttttgattctttttttttttttttgagaaaagtgaattccttttggtggtagtagggattgggggagaaaaataggaatataaatagaaatggaaaataaggcctgaagttgagaaggatcagtgatcctcaggaaaagaaaagcaacatctaatgaaggcaatttaatgtggctggtaatgggtttgcaagttacttgtattccaagtggtgtttcctcaagggatgtacacattactaagtgcaaacagctagtattagggtcatttacaatgcaatttattttaaaaaaagaatgttactaaactctaggctatctaaaaaactaggtaattttcagaattttcccatttccaactttatataacagtagattcctgtttgtaatcaaaactaattaatttattctcaaattattcccaattgtctgtttcagtagccttgagatttattttataaagaaatgaacctttcatcgaattacctttgtgttgataggatttgggagcatagaaaacattcagaatggagccttgtttaatttggcttccgtctctttctaaagatactctgttgtttttcctggcctgaagtttaaggagaataaaaacaaaaatctgttgagtctagattaaaggtgaacctgaaatgtagcattatgccctccaaggccaaatatttaacaccttgactttttttctttaaataactaattgaatagctagagaaaaaaatcaagtgagcattgataatgcctctaagtctttagttctttgaacctaattaaaaaataaaccataattgtaaccaatttatttggtgagcttcttagacctctaactatcttagagaatacagcataatggtttacaatctggcttctggagtcaggcaagcttgcgctgaaattctaattctcttgtttaccacctgggaaatcacagccaagtgacttaccctctttttgtaatccttagttttcacatccgtaaattgagaaaaaaagagccttactcataggacagttttttggactagaaaataagaacaatattaacatttattgagcatttaacatatttcgggcacttatatattcagtatgttatacgtgccaactcaataaagatataagacttttttccattacctatattgcttccaataatacgtaatacaaaagacaatgtatgtaaaatacttagcattgggcctggctcaaattatgtgctcaatgcatagttattatcatcataatagtatgttactcgatatagctacattatccttaacaaaattgtcttccattgatgatctgaacacccaagcgtctgtcaactaaaagaagaaagcacagccatgaaaagacagcaagaattaatgaaaaaaaagatcagtgagaaataagtcagtccagtgcctttgatttcagtcttggtttccatggtgactcaccttcagagaggcttttacagtttacctccacttccctttctccaaacactggtagtaaattcttgaaaatgtaaaccttaggagtagagactaataaaataatttgagtacttcaatgtgcattgaaattattacctcccttccagaggacttctgatgctacatttagaagttggctttattttattatttttgccccagattggcatcaccactgaaatgtgaatgtttggggaaccatataatacttccagaaatggatagaattgcggttttaaattgagatctacttctaaatctgtatggacctctatatgggttttgcaagccgaagaatccaggaccctctgcatttagctgaaccctgttggatagaaaacagataagagaatgagcattattttagtctaactggaagaagtgagagatgtatctcttcagcgacagggaaacagaaagttagcccgaacttcagcgaacagtaaaatcagtacaccgattctgagcttggcaaaagttcctgctttatataaatattcatcatatgttctataaaacatgcagtcccttatgttaagagatgcatgctatgatggtaccaggtaatagagtttgaatgttaaatcataatgctctacagttttcttcaggagcagagttccccagaacgtaagtatattattcggttacttcacctacacattagcatgaacagctagacactggctctgtctctagtcttgggatgaacgctgtttaagtaaatgttgtgggtagggcctttactgtggcagaggaaacaggtcctgtcctagtaagaatatttactaacaattacggagcacttcttgtgttccaggcatcctgctcagttcttctttatacaccattctgttaaacctcctgatcccatggagtaaacactcttgttattcccttgaggaaactcaaccttaggtaggttaagtaactcactttaaatcagatgactcatattggcagagcacatattccattccagggtgactggattctataaccgatgcactttccactattgcctctcatgctgaagagtatcatcccacagaaggacttattttccaaaaagccaagtgcctcttgtgtcatccaagtgcctgaatgtcaaggtagaactaatgccattttcttgaagccactgggaactgtacaatcatttatttgaagctggcatatgttctctgctgatgttcccctcttgtttgagtgaaaagagggaaaaaatgtcttttcttccccttgttaaagttgtacttgcaataatcagggccggccctaaaactcattattacttcctctacaaactagtttttaaccttgcaaagttatttcctgacaattaacatttgatactcgaaacaatattgtgagaaaattgggacatatcagttcccccagaaactgagatacgaataagtcatttggtaaaagttagacaaagtccccagatttttctgggagaccagacattccaattttgaatcctcagttctttttccttgggctaactttaatcagatgctgtattagcaaggccacggtgctctgactaacctgaatattccatgatctttgtgtttcatttactgtgtataactggaccatagaaaggtgcgaatgcctgttctgttttctcacctctcagtcactattcaaaccattggttctggcttttttctgtccccaccacttccctgaaatttataccaagtcacatggaaccaccttattgttaggttcagtgggtgcttctcaggcctcaccgctctgataattttgttggtgttgattacatcctacctgaaacacattctttccttgcttgcagctgtttgtgccatgtttttcttacttctctggctgctgcttttcaattttgatttcctttgctcattctttaaatattagtgctcctcaggtttccatctcgagcctgttttattttgatgtcacacactctttctgtatatcttcaaggactcatatggctgtgattatctatctgtagatgctttctacatgagttggcttacttcagatttctctcttgagattcagccttgcgcacccagctgacaattgatgtctaactcttttcttttccccaactcaacaggtctaaacctgtctcaaacaaaactcatcaactccatctctttctccatcaaaaccttttggtattcctgttttgcccatcttagtgataggtattactatctgctcactgaactaagctgtgaaactggatgttaatattgactcctcactttttctttccccttcccatatcaaatgcacataagtcttgtcataataacttataaatatctcttagttaatctcttgaagctccaacatcctctttgtgtaatggggaaaattgtacctaccttatgggattgtagtagagatgaaatcagataatatgtctagccactctgtcttttgcctggcatgctgtaagtattcaatacacatacactctgttactgctgccgccctctgcatccagtcttgcctcacatatctctttcctctacatttcttccagtcttgaatacatatatatgtatatatatacacattttattattttattagacttcaagttctgggatacatgtgcagaacatgcaggtttgttacttaggtatacgcgtgacatggtggtttactgcacccatcagccattatctacattaggtatttctcctaatgctatacctcccctagccccgcagcccctgacagaccccagtgtgtgatgttcccctccctgtgtccatgtgttctcattgttcaattcccacttatgagtgagaacatgcggtgtttggttttctgttgctgtgttagtttgctgaggatgatggtttccagcttcatccatgtctctaaaaatgacatgaactcatcctttttttatggctgcatggtattccatggtgtatatgtgccacattttctttatacaatccatcattgatgggcatttgggttggttccaagtctttgctactgtgaaagtgctgcaataaacatatgtgttcatgtgtctttaaagtagagtgatttataatcctttgggtatatacccagtaatgggactgcaaaccatcaattggtatttctggttctagatccttgaggaattgccacactgtctttcccaatggttgaactaatttatactcccaccaacagtgtaaaagctttcccatttctccacatcctctccagcatctgttgttccctgaccttttactgatcgccattctaactggcatgagatggtatatcagtgtggttttgatttgcatttctctaacaatcagtggatgatgagctttttttcatatgtttcttggccaaataaatgtcttcttttgaaaagtgttcttatcctttgcccagtttttggtggggttgtttgtttttgttcttgtaaatttgtttcagttccttatagattctggatattagccctttgtcagatggatagattacgaaattttcacccattctgtaggttgcctgttcactttgatgatagcttcttttgctgtgcagaagctctttagtttaattagatcccatttgtcaattttggcttttgttgccattgcttttggtgttttagtcatgaagtctttgcccatgtctatgtcctgaatggtattgcctaggttttcttctagggtttttatggttttaggtcttatgttcaaatcttcaatttattttcagttaatttttgtataaggtgtaaggaagggatccagtttcagttttctacatagggctagtcagttttcccaacaccatttattaaataggaaattctttccccattgcttgtttttgtgaggtttgtcaaagatcagatggttgtagatgtctggcgttatttctgaggcctctgttctgttccattggtctatatatctgttgtggtaccagtactatgctgtttttgttactgtagccttgtactatagtttgaagtcaggtagcgtgatgcctccagctttgttctttttccttaggtttgtcttggctttatgggctcttgtttggttccatatgaaatttaaagtagttttttccaattctgtgaagaaagtaaatggcagcttgatggggatagcattgaatctataaattacgttgggcagtatggccgttttcatgatattgattcttcctatccatgaacatggaatgtttttcatttgtttgtgtcctctcttattttgttgagcagtggtttgtagttctcctagaagaggtccttcacatcccttttaagctgtattcctaggtattttattctctttgtagcaattgtgaatgggagttcactcacgatttggctctcatctgttattggtgtataggaatgcttgtgattttagcacatcaattctgtatcctgagactgctgagttgcttatcagcctaaggagattttgggctgagacgatggggttttctaaatatacaatcatgtcatctgcaaacgagacaatttgactgcttcttttcctgtttgaatacccttcatttctttctcttgcctgattgccctggccagaacttccagtactatgttgaataggagtggtaatagagggcatccttgtcttgtgctggttttcaaagggactgcttccagcttttgcccattcagtatgatattggctgtgggcttgtcataagcagctcttattattttgagatacatcccatcaatacgtagtttattgagcgtttttagcatgaagtggtgttgaatcttatcaaaagccttttctgcatctattgagataatcatgtggtttttgtcattggttctgtttatgtgatggattacgtttattgatttgcatatgttgaaccagcactgcattccagggatgaaactgacttgatcataatggataagctttttgatgtgctgctggaattggtttgccagtattttattgaggagttttgcatccatgttcatcagggatattgacctgaaattttctttttctgttatgtctttgccaggttttggtatcaggatgatgctgacctcgtaaaatgagttagggaagagtctctctttttctactgtttgggatagtatcagaaggaatggtaccagctcctctgtatccgtgaataccgatacagagctgtgaatccatccagtcctggactttttttgattggtaggctattaattactgcctccatttcagaacttgttattggtctattcagggattaaacttcttcctggtttagtcctgggagggtgtatgtgtccaggaatttacctatttcttctttattttcaagtttatttgcgttgaggtgtttatagtattctctgatggtagtttgaatttctgtgggataagtggtaatttcccctttatcatttttaatatgtccttttgattcttctctcttttcttctttatttgtctagctaacaatctatctattttgttaatcttttcaaaaaaaccagctcctggattcagagattttttgaagggtttttttgtgcctctgtctccttcagttctgctctgatcttagttatttcttgtcttctgccagcttttgaatttgtttgctcttgcttctctagttcttttaattgtgatgttatggtgttgattttagatctttttcgctttctcctgtggatacttaatgctataaatttccttctaaacactgctttagctgtgtcccagagattctggtatgttgtgtctttgttcttaaagaaattacttatttctgcgttaatttcgttattacccagtaatcagtcaggagcaggttgttcagtttccatgtagttgtgtggttttgagtgagtatcttaatcctgagttctaatttgattgcactgagctgggagagacagtttgttatgattaccattcttttgcatttactgaggagtgttttacttccaattatgtggtcaattttagaataagtgcgatgtggtgctgagaagaatgtatattctgttgatttggggtggagagttctgtaaatgtctattaactcagcttggtccagagctgagttcacagacttgaatctttttttttttttttttttgagacagattcttgctctctcccccaggctggattgcagtggcactatctcagctcactgcaacctccatcttccaggttcaagcaattctcctgtctcagcttccgaaatagctgggactacaggcgcccaccaccatgcccagctaatttttgtatttttagtagagacagggtttcaccatattggtcaggctggtcttgaactcttgacatcaggtgatccgcctgccgtggcctcccaaagtgctgagaatacaggcatgagccactgtgcccagccaacttgaatctttaatatgcaaatctgagcatgtcactatcctcacatgacttcccattattgtgaaaataaactctaaactatttgcctggtatttacggcagaagcatctgcccccactcccatatttctccagccttatttctactttttccttcacatttcaccttcagtgctgctttctccaggaaggctttgacagtcccattacttcagagttggctaatacctctgtcttctccaaattcatggagcctacctccacacttaccagactatgctacagttatatatgtcttttttttttttttttgagattcagtctctctctgtcacccaagctggagtgcagtggtgcagtcttggctcactgcaagctccgcctcccgggttcacaccattctcctgcctcagcctcccgagtagctgggactacaggcgcccaccaccaagcccagctaatttttttgtatttttttagtagagacggggtttcaccgtgttaaccaggatggtctcgatctcctgacctcgtgatcctcccaccttggtctcccaaagtgctgggattacaggcgtgagccaccgtgcccggcccctgcagttatatatgccttaatatggactctgcaatagtctgtatggtccatgaggcaaaggactgggtttacctggaaagcctccgctatgcccataactagcataatgtctgacatttaggcgatattcaagaaatatttgttgaatgaatgaaatacttgataaataacttcaacctttttaactgataaatttttaaatgattaattttaaaccaacataaccttctaaattaatattcctttccctcctcatcatcctctaaaaggacacatgaactctctattctgctggtttaagcagatgatctagacatcaacctagatgcatcatttaatttaatttaaggtaaaccagcaattccattttttgttgccgacaatgaattaaaaaaaaaaaaacagagagataaggcctgagaagcacccattgtatttagcaatagggaggttccagtgaccttgtatagaataatttcaaggaagtggtgggaatagaagccagaccacaatgggttgaggaatgactgagaggtgaggaaatggaataggtattcatgcatttccatgggtccaattacacacataaaaaacctaaaagaatagtacattaaaatttaacccatgggccatctaaatagaaggaaataaagtttttattacttttagtttatgaactttccaggaatttttttcttctcatttagcctgggattttctctgtagcccattactatattctttacggcaacttagaaacactgatatcaggattagtttatttgtagcatcagggccacattaaactgattctaggaaagtttcatagaatgagaagaaaaacttatggctggaatatttgcctcctcagaggtcttggtagatgattcccaggttattgatttatatttctcagaattatattcaaacacagtgtgctagtttgaaatctcctttcattcaccttctttccttaatctacaaaattagcaattgtttataaattgagtgtcttatgtgtccaaggacttattaggggttgaagaagatataaaaaggaagaagtccattctctgtagatttttatgatcttattgaaggaatatccatttcatgagaagataataatacatggaagcacacattaagtgctaaacaaatagataacagggatgtctggagtctggagaagggatgggtatagcaatattaggcagtgaagatggattccaaatgggccttgcaggacagacaggagttgggtagtttgagaggagatgtatcatattctaggcaggggtcatgggagatgcaaagtcacagtgaagttacaatataaggctgattataactggttaggatagagtattcatctatccttttacagcatgctgaagggggaaggaatattagagacaggcaagtgaggttcttaagtgttaggactttagactctcggcataaatgtgtacaatttttttgagacagggtctcactgtgtcacccaggctggattgcagtggcacgatcatgactcactgcagccttgacctcctgggctcaagcgatcctcctgctcatccttctgagtaactgggactactggtgtgcaccatcatgcttagctaatttttaattatttttgtcattcaagctggttttgaactcctgggcttaagtgatcctcctgccttggtgtctcaaagtgctaggattacaagtgtgagccaccatgccaggccagaaatgtgtaattttgaaggtttttgtttttgtttttttggtatgtgtgtgtgcaagggttgcagtttgggcagaagaaggacatgctgatgggtgatgcatcagtagaaacaaccttgtggagttgtaggataaccaggatgagaagggtattggatacgattaggttagaagctcctagctcctcagtgaagtggtacataagggctctctctgctggtgtgtcatttttatctgctagattctcaaacattttggtctcaggacccccttgtactcttaaaaattattgagaataccaaagagccttaatatatgtgatttatatctattaatacttaccattatagaaattaatgttaagaaaacttgaaaatatttaatttattaaacataacaataaacccattccatgctaattttattttttatttttttgagacggagtcttgctctgtcgcccaggctggaatgcagtggcgcgatctcggctcactgcaagctctgcctcctgggtgcaccccattctcagacctcagcctcccgagtagctgggactacaggcgcccgccacgaggcctggctaattttttgtagttttagtagagacggggtttcaccgtgttagccaggatggtctcgatctcctgaccttgtgatctgcccgcctcggcctcccaaagtgctgggattacaggcgtgagccaccgcacccggccaaggtgcactcatttcttgtggcaactccccagccctgagttgaagaagcagcatgccctccacactttatcctcagaaggactgagtgggaaacccttggtgtacatcaaggaaacaaaggcttttgatcttggcaaaatgggataatgcccatgggattccctagcagatgatgggaagtagggtggcctctgccccctgaagacatgtcaggggtgggaagcgaaggatctagattgggtgtctgtgctctgacaccacaccacacttttaggagtctaaaagaacaccttctcttttcaagacaagacctcgaaaagctgaatatggaatggggtagggctgggaaacttgtccatcactatgacaaagacagaaaatgttgtaaacctcttttaaaaagattgttgtagctttcaacattctttctttttctttagtaaaatctctaagaattccagcctgccttttgtttaggggaacctggaaaaagagattttaaacaaacaaaacatgacttaattttatttttcttaaaagaaactgaaatacaagtacattgttaaaaactttggcagatataaagaagcagaaaggaaaaaaaataaaaccacacataattttaccactcagagataaacactgttaacatttggagcacagatttatccttatttatagcagcattctctatatattgtttcataataatctttttatccctagcagtgcatcataaaacaaggcatctttatcctttaaattgtatattcaggaaaagcgttcttcatcttcccttcaaaaagagttttgagagtgaacgtaagtgtgtgtgtatgtatgtgtatgcatgtgagtgtgtgtgtatatgagtgtgagtgtgcatgtatgtgagagtgcatgtgtgttggtggcatcagtgccagagtgacttcagtaagaagtgtaagaaaacctgaggaaaaatggaaaaattaatcttggggaaacagagttggggtcacttgactggccacctcttaaacctagagattatttttatagagtgttcttggcttaaaccttagaaaactgtcttttatcttgatagaataataatgagtaaactttgaagaggactttaaagaggggaaaacttcaagagaaggtggagagagagattcagcgtgtgtatatatgtatacatatatatatgtatatataagtatgtatgtatatatcagggagatttggaagtgaaacttgagtgccttgtaattgaggcagtttcattattgcaggaagaacaaatctggaagcaatttcctcagtcactcgtgctttgaatgttcctgctgtgagtgtggaacatgtaagtcaccaattccattgttcagtgagccctaaaaggataaattaatgggacggttcacttgtttcaagggtcagaagtggtctcccaattgttaaattcaaactaccattttaaatgtatgtatttcaattgctctcttgggatctttggccagttgccatattcaaaacacttttaggtatcccaatgattatttaatgggaaagaaacctctggaatcctggaattcatatctactttagtatttattaagatgattactaatattgctatgtacctggcatgcagtagtgctcggtaaatatgtgttgaatgtatgaatgagtaaataaatgaatgaatctctcacttgtcttcacagcatttttggagccaactgcaaaaatggtttaagttcatagctgcaaactaaacaaaattatgaaaaagcaaaagaaatgaagtgcagaaagcgaagtatacctaaaattctcactctggtgagaaaagttggctgatgtttgaactgtaggatttttttttttctttcttgagccggagtcttggtttgtcaccaggctggagtgtagtggcactatctcggctcactgcagcctccgccttctgggttcaagcgattctcctgcctcagccttccgagtagctgggattacaggcatgcaccaacacgctcaggtaatttttatatttttagtatagacggggtttcaccatgttggccaggatggtcttgatctcttgacctcatgatctgcccgcctcggcctcccaaagtgctgggattacaggtgtgagccactgcagctggccggacttttttatttgtataaatgtaagtacaagggcagttttgttacatgaatatattgggtagtggtgaaatctgggcttttagtgtagccatcacctgaataatacatattgtgtccattaagtaatttcttattgtagggttatttttgaagtctgtctctgagtgggatcctaaatatggtttaacagttaatttaattgtgtgtttcaactcactattaggggcaacaaagtgcattctctgccagggtctcaatgattctttcattgaatccttctcagtgttggaccaagacatagcagccagaatactggcttacaagtcaaaaggcatgagaacacatatatctgaattctgtctttaacacttttacattgtttaatcttgtaccgttagaaatttacctttctagacaggatttcttcatccaaatttaacctaaaatctatttaagtggttgattaaagaggttatacgtgttaagcatatattatggtttgctattactgtagtccttatatgcttcccagaactcttatgataactattgcttataaaatttattcaccaactaattgcatactcatttgcgttactgatctctccattaatttttttgtgtatcatgtcttatttccccaagaatcagatgcatcaaaataacaaacttagcacatgtatctcctcctctaggaagccttccctgacctcattcctttaatcacatgatatgttgtgcatatttttatttgcttatctgttttatatgtatctcctttctatgctgaatgatgagattgttgaagatgaaaaccatgtcttattctcaacacctactggaatacctgactcagagaaggtgtgagcaacctgttcagttaatgaataaatgaatgagaatgaaggagaagtggaatgggcttctataagagcagaacagatgaagattataggtggattcttattaaacctcttacttgttgcatctctttaaagaacaggagatcgccacttacttggggtgggggaaggatgggtttcatggctttcatcacagctgcatacatggcaaatttcagaaagtagctgtagttcttactgaatagcaacagctgtagcaatcactggcagggcagcgcagggtagaggtgactaaaataaaagaagctagacagagctctttcagacttggattctaaccccagttctgctgcttgctccctctggggctttgtaatggtatttaaagttgattgtcattgagtctaaatgcattacaaaaatctgtgtctccctgcctttacctccacagagagaatacaaccacacctagcactttccttattgggaatgtgagctactcagcctcccacagaatctcaatctattgcaaaacaaaatctgaaaccatttctggggtggcttttgaatgggatgatatttcatttctggtgtgatgtgaaggccttggtataatggtacaggatcagatggtcttactgtattgtattatttaatgggtgccacttgataagacaaccaagcagacaaaacagcctgtgtgtggagaacaggaagaagtgtggaagctttaaagagtggaaaggcttatctttttgttttcctataggttatgatgtagcattcattcactagcttttaaaagtgacttttaaataattactttatgtatccatcagtactcttttatttgtaagagtgaaaaaaaatcaactttaattagcttaagccgaaagggaattcattggcccacataacttaagagtcagggtgcacatcagctttaggtacagcttatccagggtattgtgatggtattgaaaatctgtctttctccacttcttagttagccttctttcacatggcggcagaaatggtttaagatgtccccagactcacagcaggactagtggagaaagagtttttcagtagcacaggtgattataggatccagactgctcctatgcttaccaaaatcacatggaaggcagttcccatggcagtaaggaggtctgttactagatgatgggacaaaaacccaagggttacatgtctttcatcattccaggaaggcaaaaaccacagacatctaccaaatttgactggtctggattgtttcttatgttttgagcttaattcactcgggcttatatactggctctttttcgtgcttttgcacaaatcgtgtatgcattctcaagcctttgttcttctttctatatgctgattctcctgatcagctccactactcccactcagctatgacctcattgcagaagaagccacatccttgtctctggtctctacctttctcagggaaagctcaccaatcatttgcatttggggtcccaggagcattgcaacttgaaatgtctaaaaccaaagttgccaccatcctgccaaatgggcttctcttcctaggttccctaatcacagtgttgcagaactttgttccttagttcagcttatactgggttcttgtcacacgaccagaaaaaattaggcacatagacacattgaagggtgaggagaatggaatttattgggcgaaaaggaaaaaagaaaaaaacgacctctcagcaaagctagcggagttcctgctaacaggcccccatctcacagattggttccaggtcacaaacacaggaactgaagagggcaggctcctgccccttgcacgcacgggaaattcccatggctccatcctattctcccactgctcaggcaggtagagattctccgaggaccctcccttttatctgcctcctgcatctatcaacagcagtagcagcatcctctttctactctcccagcattctatcgtcaatgttgtttttgactatttcctctgcatatcatttctccctcatgtgaactctcatagttctttattattatgttttttgtgaatttataatttcctctctggttttatagaccttcatgtatctaatttatttctgccacagtgatttaagtttgctgatggaaggcagttttacttatttatctttgtactatttccttcctggttgtcatcctccagctttccccactttctacttctcccatcaatattatacctagacttttatggtttgataaatatttgtagaataaaggaaggagttaatgcaatctgtagtataactacatggctactatacttgatattgttaaaaaaccagtgcctaacagcagtcgaagtggtaaaaacagatttcattcaggaaacattgcaataggggtatgttagagttctccagagaaacaaaaccaacaggatatatatatatatattttttctcatatatatattctcatatatatatactctcatatatatattctcatatatatatactctcatatatatattctcatatatatatactctcatatatatattctcatatatatatactctcatatatatattctcatatatatatactctcatatatatattctcatatatatatactctcatatatatattctcatatatatatactctcatatatatattctcatatatatatactctcatatatatattctcatatatatatactctcatatatatattctcatatatatatactctcatatatatattctcatatgtatatatactctcatatatatattctcatatatatatattctcatatatatattctcatatatatatactcatatatatattctcatatatatatactcatatatatattctcatatatatatactcatatatacattctcatatatatatactcatatatatattctcatatatatactcatatataatattctcatatatatactcatatatatattctcatatatatatactcatacatatatacatatatatatacatatatatatacatatatatatatatatatatatgtatatatatatgagagattatgaggaattggctcatgtgattgtggaggctgagaagtcccaggatctgctgtctgcaggctacagattgggaaagccaatgctttaatttagcctgtgtctgaaagcccgagaaccagaggagccagtggtgtaaactctagagggagggttgaagaaactgaaatcaaatgacccagttcaagcagtgaggaaggaaaaaagggacaaattccttcctctgccttttgttctgttcagaccttcaatgggttggactcacactggggagggcaatctacttaacggagtccaccgattcaaatgctaatctcatctggaaccaccctgcagacacctcagaagtaatgtttaacgccctagaaataatgtttaatttgggaagccagtggccagtcaaattgacacaaaattaatgatcacaaggggaaaagagacctcagtatggaacttggctcaactccaaatacaacatggacaagtagggatttacagccaaggaggagcaggggtaggagtggggagggggtcagtgaatggaaaattactaagaggaaacattaagggtaggggggattttggttaaacagacctaacaggattcttgctgaaggcaggaccagggtggtcagacatggagggtgagggaactctctttaaactgagttagaaggtttcttgctaacaccaggtgatgcaggtcctgtaaggatggatgctagggtcgaggcctgggctcagaggagcctgactgcagtttagtcaaggagagaatctttgtcagtattgaagctttcagattcagcatttattataaccagatctcaattgtgtaaaatgctaaggaaggaacttgccctaattccttgagcttccttcatcctctctctgaaccagccccccactttcccataaggacatgtttaggaaagaggcagaaaaacttctgactcataaaaactggtgaggaagaaggaagagcagaagcggaagccaaaatgcacataactatttgctgaaacggttttgagcatactttgcggtctcttttgtaatatatacctagcccctacctataatgctaccttactttggtgtttcacacatacaaggcccatttgaggtcaccctaatgtggcaagagaccagtgaataggagcttaacttcaaacttagagtctttatgaaaaatgtcaacgcgctgaacacgaggacatctgaaaccttaaaaggccacagcaaacatcagggcagcatgcttgagaacagcagtgtcatagatacaagcatgaaaagaagtaaagctgtcataaatgtgaaggagctggcgaaagcacatggagttttattaaggcagatagcattccgtacaatcatcctgaatagttttctgagtagcacttgatgatcctaccctttccgtaatttgtttgttcattgttttggaggtgaaagggtgtgcttgtgagggtgtttgagcctccttgtccatggtataaccggttggttttgttgccctcactcttcaggcgctggactctcatcctgtttgttcccactctgggaggcaccaccctttctaagacaactgctttcttttgtttctggttcaactttgttaagtcaaagtataataaatatcaagattgtttgaagccagaaataggccactgtccaaaattgtgttcattccacttgctggtagatagctattttaaataagtcaggcttgccattaattcagttctgctgcactgttcgtattctgttctcattctcttatcatgaggggagctttgagttttatcatctctacatctctgtgttcttcgtggtgattggacagttgccactctccatgactaccgtgactatccctgaccacctgtccagctaaaatttcaacacaaatgctgcacagctaagctatctgatggtattaattatatatttatttgactgcactgtttctttttttctaattgtatgtaggagggtgtctttaatgatgtacagttggcaattctgacatccttaagggtggtggcattcaagctcgtgactgccattcacagcaagaaatacattttacatcatgatccagtatacatgttcatgttatatatgatgtgtaactaaagtttggttaagcaacacttaccctattatgtgtgatatggtctatagtttctattattttgtattttacttcattaaaaaatgcagactgtgccttagcaagtgggcgtcctgattcaggaatgggttacaatttgagtctgaaaaagaactgcttaacagtcatcaggtattagggagccttagcatgctaattctgaaaagtctttggagattatctataggcatcttcccgttttcctcctattttacagttggaggaatttaaagtccaagtagggtaagtcttccttgcagggaaacagtggtgctctctgtgtgtgctatcatgtgctatcacacctatttattcctggaagcacacagtgtccagttgaaccacaagggctgtctactttttgaatctagcaattatgcttctggatgccttcctggtgatgaataaactgttcacactttcccttccccaattaaatcttgaggtattttctccctccattagtttattatacatcattctgagctaaagggaagactgttaacagggcattgagtgtggtgtatcctcttctatggatggcatgcagctatcattaatcataaactcaaaacagtgggttgaataaggaacaattttaggtacatacttagacattaaaaaaatctaaatgtgcattttctgtaaggtagttgtaatcttagcgttggacaactgcatagatcttggtgtatttgattatttgatgttttttcatttttcctttataaacagtagtgatctccttgtaacaaaatatgacattttaaatttcattgtttctgtagtaaagggaattacagcatcttatggtcaaacaattaacatcaactttgagttattgataccattcatagtgtattagtcagctcaggctgccataacaaaataccaaagcctaggtggcttaagcaacagaaatttattttctcatagttggagacttgaagtccagaccaaggtgccagaatggttgctgtctgtgagggctctctccttgggtcacagagagcattttctcgcttcctcatatagcagagaaagagcaagtgagcaggctctttgatgtctctttttataagtgcactaatcccactggaccagggccccaacttcatgaactcatctaaacttaattacctctcaaaggccccatctccaaataccatcacactggggattagggcttcaacatgtgaatctggggagacataattcagatcatagtacatagctaaggtcttattggggtttaagcattggctgaagcatgcaaataacttgcctaaattacgtttattaaagacagactcttccatttgagtgaatggatatttttcacactataattcttatgacattcagtagcatttattttatatttttcttaataatgcaagtactgtatatggaagagcttacagttaaaatgtgagaacaacattatacctttaaaaaattatttttgcagtttttttcctaatgtagattctagtccttgttaatagttaatgaatttctaaacagcccagcactattttcaaaacttaatatagaaaagttacccaacgtcattgagaataatgcatattttaatgaaaaacagtttgtgcaaaagacatattttaaaacaactttttatatttatatttattgcagtgtagagaagagaggaatttcttagcatgaaactgtagggagccaggtatttgtctacaagaaatgctatagagagccatgccagtgacacacacaaaatatccattcaaactgaagaaaagtaaactaagagggggtactgacaataaaaaattgttttaaaaccctttgtttgaatggtgttggtgcacccctattttaaataaagtccttagatatgcccaaaccaattcttatttattttctgctgtaggaaagctgttgattaagaagggatttttttttttctatttttcttgtttctaggtttaaactggtgtgggctgttttacaactttaggggcctttcaatttctgtggtgggcttttgtataaagagtgctgtgcatctagatgaaatagtctgtgtctctttatagcatctttgcttatgtgcttttacacattattatcacattttttttttgtttattctatttcagtgtttaactgctgaagagatcttttcccttcatggcttttcaaatgctacccaaataaccagctccaaattctctgtcatctgtccagcagtcttacagcaattgaactttcacccatgtgaggatcggcccaagcacaaaacaagaccaagtcattcagaaggtatattagaagcattttttctcttttatttgaaaagtctatgaaccattgtgaaatgaagttaatatgcctctttttctgctttattgcttttaattaatctacatagaacttcttcgaataatacttatattttgaaagtatttggaataagaagaattataactcatttaagcttcataagttatgctcttctctttgttgaatcaaaattacatttagtacacttaaaaactttataaaatccagttcttgattttaaacaatatatattttatgctgtgcatattggattgcattttctttggcttataattgatcatttcatttccattatcttttgcttaccatattattttaaagcttgtcagtttatctgcagttaccaatgaagagataagagaactagcatgcttctagattaaattttcagggaccaaattattagttttaattttcataggtgaaagaaaataccagagactattttaagtgactataatggtatttccatgaatatgtactttaacggtatgatctgatgcaaagaaattttaaaccataataaaatatatgtatgcttaatatacagctcacatgcttaaatgtgtagaagggtcactttcaccaatgagataaaccaattgttcagggagtctgtttgaaaattatgttggaattttaaagtttcgttcatttgttcattaagcaaatatttattgaacgacataccaagtattgttctgtgcactaaggagtcaatggtaaacaagaagaataattatctgtctttttagggcttacagtgtagtggagaagatttaaacaagcaaatttaaacaagcaaataaacaacatagtttcagagtgaaaggtgctatgaagaaaataaaatattagtaggtgagggatagaaggtgatagtgggtgagtgtcatcttagatagatcaaggaagccctctctgatgaggtatcatctgagcagaaggagagggccacagcaaggtcgtctctagtgaagagcattcttgattgagggcacagccaacatagcttggcaatgagcttggcatgagtgaggagcagcaagaggctcctatgtgtctggagcctagtgagttggtggggaaggtagaaggatatatgtcctggcgggagatggagaccaggtgaggaagccttgcgggaggtggtaaattgattgggttttattctgaatgtagtgtgaagtcactgtaccattctaaacaggggaatgctgtgatctcattcaaatctgttaaaagggcattccacctgcctacaatctgaagaacagattgtaatggggcaagagtagaagcagggtgattggtcaggaagcgattgccaggagctgatgccaggatgatggggacttggactaaagtgggggaagtagaagtggggagaagcagtagattcgggatgagtcctggagatcatgctaaatggtttgccctggattgattgtgggagagggaacaatgagtcaagtcttgagccagagaaaccggagagatcgttataccatttcccgggatggagaagactgggaagagccctttattgtagaggaagggagtggtgaggataaagaatataatttttggcgtggttgctttgagacattgacatacacaggtagacagggaggtccagtaggcaaaggcacacagacatttggagaggagattgctttcagaggagaggtcaagactggggatttgccagtctagagatgatatttaaagacactggggctaggcacggtgactcatccctgtaatcccagcactttgggaggccaaggcagaggattgcttgaggccaggagttcaacaccagcctgggaaacatagtgagaccccatcacaacaaaagttaaaagaattagctgacatggtggcatgcacctgtaggcctagctacttgggaggctgaggcaggaggatcacttgaacataggaggtcaaggctgcagtgacctatgattaccccactgtactccagcctgggtggcagagggagacctcatctcaaaaaaataaaaataaagatgctgttctgagtgagctcatctgacaggagtatagatagataaagagagtaaagatagagaagaaaagggaagacttaacaaagaaagtgcctgttttcatttccggattcctttgtgcatgtacacttcagtctccctctatatgaataaaagaaataacaacaacaatgacaaaaataacaatgatgatagccaattcttttgtggtacttactatatgacaggcactctttgaagtactttacatattcatatattaatttcatgcaactatatgatgtagacactactattcccattttacagccaagaaaacaaaggtacagggagtttaaagtcatttgctcaaagttacacaacttgcatgtggtagagccagattcagcccaggttgtcttggcttcagaacccatgttctcattgatattctgtatttatatatacaaagcatagaaatcaccaagtatcatttatcaatataatttgcaattttgtgaataacagtaatttcaattttgaaatattagtaattttgaattgctaattgttcaagcaaatatttttgctagagtatatttatatttctacaagcctgtttaatacagtttgcactttacattaaaaagtaaattcagcaaacgctttaggatactacccttgactaatagtattgcacagtaaaagcacagactctgaagccagtttacctgggctcaaatcccagctctgtaattttccagctatgttgagaacatgactttaaccttttcctgcctcagcttcctcatctaaaatagtacctttcttatgggcttgctgtaaagattaaatgacttaatatttgtaaagtacttcgaacattattcagtacctaggtgctgtggtaggcctgttagttaggtctgttaaataactacatctaacgtgcataactctaggtacaggaagtgaggtttcagaaaaagcaaatactctactctgtgcaggttacttacttatattcaaaagtaattttttaaccccagttgctctgtgcaagttaattactcatattcaaaagtaatttttaaaccccagttgactaggttactctgtttcaggaaccaggagttgagaactgggaagtcagaagcagggtgtgagaggtagaattctagggacaacactacaaagtcataaaatggaaaagtcagcaattctaagttatgggatcaccaaatattcagacagataacccaataaaaagagaaatgcctcttagggtgcatattgtcttaggaattgaaatgaggctggagtggagtgggatcggggggagaagatatggaaatgtttccaggggaatttagggaaactagtagagtgcttgttactactcatctaagatgtttaacttggcactctattgcctttttgatgtgatatcttgattgttttattttagtgcagctgtatgattatttaatatctctatgggtcagttactttaatagtacctttatagaattgttgggagtatcaaatgagttaatatacgtaagtgcctagaacaatgcctggcacagaggaagaagaatgtctttgttcttatacatgtgttcataggttcaccacattccctggcccctgtgttatcctattgtttttctcactaagaaaaacattttaaaataattaaatgcactggatttttcatataacttttcacttaatgaagagtcaagtctttagctcatccaaagcaataaaaaatcaacataaaaacaatccaaatgatacaaaaatttgaatacatttatgttgacatttcagtgggtagacagtaaaatttctcagaggcctcacattttatttttgaaagtatttcatgtgctttataaaatgatattgatgagtacagtaaactgatgataagttaaattctctaggagagaaattaattgcattttagtaataatttaatggtctgaaattgcctcactttttgatgaagttgcagaatcatatttaattcatatagtagataaaataaagaatattaattgaagcatccttggagatcatattgtccaactctgtaatacaactagtgatgtaagtccaagttccagagaggttaagtggcttcttgtaggtgtcgtacctaaggtgaaatgagagctcctgattcttagtttgttcttcttttcactgagtaacagtgaattaaatgaactaacatttatatcatggtctacttactaattttttaaacataattgtaatcaacatttattaaaggcttgctatattccagggacagcattaactggttttattttattcttgtaataactctatgaaataggagctattttaatctgcattttaaatatgagaaaactgaaacttagggaagtcaaggaaccaacctatgatgccagtgaggggatgagctgggatttgaacacaaatcctctggctccagagttcacactgttaaacaccattctatacagtttcacagttaaatggggccaggcaaaaattgaagcctgggctgtatgcaggtctgcaaaatgggtatcataatagaaccatatttcctcactattaaaatgggaaaaatagcagcacctatctcatagggttgttgtgagaattaaatgagttaaaataggtaaagttcttagaagaatcttacacagtggacagtcaataaatgtaaactatcctgactgtatttctttgaaatctattaaaatctttcagggttcacttataaggagactggtccctctctgaaacagatcgtttgagaaagtcccacgtatatatggagatgtccaaggaatagattcatactaatgtatacttttggtgcttcagttaagtggaaagtgcttcagttgaatggaaactccagacatttttttctgaatgatgttctaacaatacaagaaggggaagctatgcagtgtgcttagaatgtgatgaattacactcccaccatcccccaagttgtgctgataaatgtgcaagggcaaagaaagttagaaagttggataagcagatcgattggtatttctttttgacagagttctgctgcaccttgaaatggggaatttatcaattcattcagcaaggaatgtgtgctttttcgtggacaattttctaggcactgaggatgaataaaacatagcccttctcattgagaatcacacagtttatatgatgtaggcaaatatatcagtgataaaattactgtagcacacaagaagtggtaagtgaacaaagagtaatgggaatcctgaccagccagggagtactgcctggagagcagggaagcttcatagaggagatctttgtgttggcttggaaggccacgtatgactttcttaggcaaggaagataggaaagtgttttcagacagagaaccacagtgtaaaggtaaagctcggttgggaaaacatagtgtttgggtaaaggccatgttgagaagggggtaaataacactggagaggctgtgcaagggtcagtttatgaggggcgatttcaaatgggggcatgagatgatcacttttgtgatttagtataagtctgtaggccatgtcagctagtaggaggggcaccagatcgataactgtcatgttatataagatgagggagtgagaggtgatgagggtctgacttcaagaccactggcaatgaggatggagaggctagaggtgagagatgcctgaagcagaactggtagggtttgatcaccagttgtctatggaattaattggtggaaagtgaaaaactgaaattgatcctactgttcctgttatttatgtaggtagtgaacaagggtaatggaacaggtgtttgcagggaagctgatgagtttaacatgtgcatgtggctttgcttctattggcacttaacaatttctttgccttttaattttagttttcaacatgtatatatcttgtctacccaatgagattataaatatttgttgaatgaatgaaggcatgtaggctagtgatggatagaagtatgtttgtgttaagagaaattccatgtagatctaccacgataaaaggtgtggaattgagaactctctccaagtggaatgtctgtggggactagtagtattgtttaaggtgataaggtaataaatgggaatgaagtgggctgttaggtgaagttgtagggcatggccaggacctgcaactgataggaaagtgtctggagaagatgcagtcgtcatggtcagtgctgtgatgattctcggggttacatagtaccatttataatctgaccttttcatttggtacttagcacaggtgactttgtatcactctgtgtgtgtgtttgcacttgtgtgtgtttgtgtgtgtgtggtggcaggagccatggtttggtccacgtcatctaggctgaatgaacatttactaagtttgtttatttgtacatagaaattctagactaacaataattatagtgaacaatctcgtgttaataaagtatgtataattcaggaaaacatttttacagttttattgagtcctttgggcttttgaaaaatgagattatctctaaaggaataataaagcatatgctaaaaaatgtgggcctgggagccagactgtccaggttcaaaatccagctttctatctgcgtgattctggcaggttacttgaattccatctgctttggtttccccaactgtatgaaggaggtagtagtatttctcttacagggttttgtgaggttcaaataagttgacatatgttgatcacttagcacagggcctggcacatggcaaatgctccctgagctgtaggtgttactagtgtactccactgacagtgccatggagaaaagtggcccaccatttaaacatgctttctggatattttaaagaagtggctatgcagaggcagctttctcctctgtcacactgtactttgtgtacagtgtgtggggccagaggcaaagtcacatgttgcacagccctgaggccacgttgtatctcagcctgcacccacacataggtgactgtcctgggtatgtttcacttgtgttcccattttgtccttacaccagcccctttaagatagagagagggaaattgaggcccaaaggcaataggtcacacatgccattagctatagagcttaggatagtattcattcattttattctaagcaatacttattgtgtgcctactgggtgccaggccctgggtctccagagatgaaagatgcaccacctttttctgtcaccagcatctcttctgtatggaatatttttgcctaatttgtgtctcacagggtgggttatgtaatgcagctgctccctcctgccccatgtgctcacttcctgagtgcttattgcatcagactgactttgattcccaggaggctccagctggggacctgaggtgctgctggtgccaccttctctgaggagatgctgtcctcaggagtctgaagacaaatgattgtgacaaactcagtgttagcagctatggtcaccttgaagctagctcactgtgacccatctccatccctgccctcccacccatcaccccatacctttgtcctgaggctcacactgcccctgttttttcttttttttcaaaatgctgttttaatctattccttgagccatagatgaaactaggtggcttggagcctgtggatctcagattgtttgtcgctgttcgctgttctagatgttctccaatttccctgcaagctgtaaaactcattaatcccataatgatgatttagggctgattaaaacataaacagtattactattaatgcttgctatgccatatttgtattctcagttttctatatacttttcattgagtagattttgttcttttaaagaagatatgtgtcttaagcttttaaagacacttttaagcttaatgtacaggaaaacatcatttggttccttgctctgattaatttctactctcttttccctctgacccacagtttggggatatggattcctgtcagtgacgattattaatctggcatctctcctcggattgattttgactccactgataaagaaatcttatttcccaaagattttgaccttttttgtggggctggctattgggactcttttttcaaatgcaattttccaacttattccagaggtaaggatgttggctatttgtttctgtgaataattgtttcttttgaacgttttagcactttgtatgtttttagcctccataattcttacagcgttttatggtctagtttatagtgtcttggataagagaaagctgaatcacagcaattgtaaaggcttgtcccaagctgcagtggatatcactatcagagacaaaattaatattctaggaggccagccactaggacgtgctcagacagctctgggacacctcagaaggccttaaaactttattctgccttgcacttggcaaatttaatttttccacacactgtttcattatttatttatttatttatttttctaaagagcatgcaatgtcaactgtactgcaaatgtcaaatgtaaatttataatgtatttaacatataccactaaattccctcatatatttactacagttataagtgactagagagtaaatgagagcatggttgacaaaccttttcattttttccaactgtggaaaaaatatcaattgaggactaataaataaatagcaacttattttttttctttgataaaatgtgttattgatctatcttgactatagtgcacactattcaatttatttcaaatctgtgttgtgcccatataaggaagagaaatggaggaagggaaataaatgaaaggaaaagagaggcaatgttgaagaggaaggaaaggagaaagagactttcatagaagggatccacaaaggggaactagagaaatatatgtatacatggactgtatctgccacccacaatgcacacacacagggagagacaaaccccatacagagagaaaatattaatagtatttcagggagtaccacccttatctcgtaatttttagaaaattataactaaagcttcctttaatttttcaagctatacattgaaagtgggtatataactttttagagttatagatttattatcattatttgatctaggacctaagataatatctatttcacagcctcaattgtagagataaaaaatattaaaatttagggtggttaaataatttgattatagccttacaatcagctagtaacaaagctaggatgacaatacagattttctgatttaaaagagtattatatacataatacatttaatatgatataatatatgtatttattatctcatctgctttatgggtcaaagtaaattaaacaaactaatactgtatgtggactaaagactctacattgatgatgttaaggttttctattttcttgaagtttaacaatataaagaatctgtctttgtaataatctctcaaattttcatagagttttttgtagcaaaatgattttccttttcttatttcactttattttcataacctcaatgttaggtaaatggaacatttatatttatatagccattttgcattgtataaacaggcacaaagaagttattcaaatgaggatgcggccatgatgtctccccagaggcaaacataggacaagaacccagacatcttctattctgcttcagaggggagatttacaatgtgtggaatggtgtaaagttgtggcttttcacccattagtctctcctctgattctatttatttgaagtttatgtgtcttgtctgttaggaacatcttgactctttataacttcaaactctaactccagttctttgtcctttaaagaaagaattattccattcttattgctgtgtgtgtgtgaaagagagagagagagtggtggcaattttaaatcttctttttgcttttattcttgggcctaggccttgatttatttacctttttaggatttatcacagttctgtaggtataagaggatttctcttgggaaaacagtaatccaagtggcctgccatatttgttttaaaagtttgattctggatatcatggactaatatattacttttagagacagtaggtaatcaatatctcatttatcttcattatctagacctaatatcgtattctaaagatgagaatcagagctgctgctttactgctaaaagtttactctttcaaggcaaatattgctgtaggtgttataggtgggaaactgtaagattctctggtggctttatttaatgagacttctagttgtaaggtttttgtgccatctcatacagttttgtcattcaaatttcaaagtttttgttttgtgatttttggcagctaagtttccaatattacttggatgtgaagagagacttagaacattggtgagaattgcatactggtattattttccagaaatcaagttaaatttcttggttttacttgaattgcccttttttctcccttaggcatttggatttgatcccaaagtcgacagttatgttgagaaggcagttgctgtgtttggtggattttacctacttttcttttttgaaagaatgctaaagatgttattaaagacatatggtcaggtaaatggactttatttaaaacatccaatattttaccccctaaagctactttatataggcaaataaagacagaaattttatttttagaattatgagaatggtccagttattaggcttgtgagtttaaggagaacaaagagtcaatatattgacaagtataagataaatacttgattcatgttgtaaaatttggaggttatttttgttttttttcaagtatcagcaatttaatattgaataacagatgtgcataattcatattctattcatattcatcaaaataagagtgtatacgtgttctaacacgaacaaacagtaacagcagaatctaataaatgaaaatacatgtaaaaaagagaaattttatatatctaaaaatacttctttacacatagaatagacatacaattttcctcttataaataaaaagcagtaaactctatttccttgtctgtatgaatcatcttttttcttcttataatctctttggtttttgttcccatatagaatggtcatacccactttggaaatgataactttggtcctcaagaaaaaactcatcaacctaaagcattacctgccatcaatggtgtgacatgctatgcaaatcctgctgtcacagaagctaatggacatatccattttgataatgtcagtgtggtatctctacaggtatatgttaattccatttttccttcattatactgcattctttatactgtgtaaacatgactgtttatgcagctaagtcagtttttgtacatttatgtatgtcagacttattcctatatatgtggggtaactggaatgtaagtctgcattgtttttccaccttccctgtatatgtttactctaaacaagtgagttaagaaagaaattgtggatcactgttctggacttcattattctctaagattatttactatatgagagtctctcaattgcatttatacaatattatatagcaaaatttattcagagcatttattttacaattctttgaagatccagaatcaaataatcctttgattgattttgaaattgcaagaagatttgcaaattattaaaattgtttttattaattgtctgacttataatttatatttatatccacatcttctaaataatattaataggcacaatttcttgagcacgtatcatataccaggtaatatgcttagcacttaattatttgatctcatttactttttataacaactactcagtgtagggattatttcttatgtattagaactatattggtttcaagtgatagaaatcaaacaaaattatatatgcacaaaggacaacttactggaaagatactagatgactaatagaacagccaagctacaggaaaaacaagaacctaggaaacattagaacccctaggactctccctccttctctttcctgtctcttagtatcattcttttctttcactgtaggctatcttttccataagtgggatacatagccactagcagttcctgaggtttgtcttataatttcagctactagaaggaaattgacctgactctttcagtcctaaatccaaaatctctggcaagtgtcttcctggttcagcttgaattagatgatcacttctagactaatctaagtgcttagagggctgggcccctttaatggtatcaagggaatgtggatggagtagaggagtggttatcaaaaagggatgctgaaaagatagtatcactggtgtctacaacaattctcatttgacagatgaagaaagtggaactttagatgttaaataacccctccaggtttactcagctagtgaatggcagatccacaagctatatagttaaacatgatataaaagtgaacagctaaaaacaaaataaaaaggaaagccagttaaggggaaaaaaagcaaaaaaaatagagcaatctggaagagattattatggcaaatacctgagtttccttctggaaagaaaacatgattggtcatatgcttctctccacctgattaaggaaaatctcaagtgtatttgaagagccagttctgtgttagcactcagctaagaggaaattattttttgcagtgtaatgaatgatgctttcagccaagagtttgcaaagaatctggaaatgttcaagtatataatttcttacaataataattacaattaacattcattaaatgctcaaggactatgcctagaacgttagatgaattacttcatttgatcttcctaataacctagggggcaagtgctgttgtaattaggagagtatgacttagagaggttaaatagcaaacccaaagatgctcagttcctaatggcaaagcttggattagtaccaaggctgccttataccgaagcctgtgttcttatatactttgctggactgccttccttatgttgctgctctactgtaagcaagatgacaacatcaactgttaaatatttattttttgtagtggggaaaaataatttgacccaaggatattgctttctgaggatatattatttaatgcgaggcacagcagagaaagcctaaagaatagtagttaaaacccttccttataccatgtgtctaaattctagttgccttacacaagcttttggcaaatactggattatagtgagattgtggttgaactctgacaagtcattcaagaggtgccagtcagctatattgctaatttaaacattgattaataattctttagataccagatggcagagggataatctgccatgaaatttggagcctaacttgaattcttgctctgatggaagattttcctgtatctttccagcgttaacaagataactttaggcagggctacacttctacagagaaggagctttgaacctggtccaaccttgaacaagttctcccctgggatcctcaagtcctgtaacacaaagcacggaagagatacagtttgagatttaaaatatttaaagggctgatcacctccttgaccatactagaaactcagtgtgacctctcctgatttcaggaaagttccttctgatgaacctactaggataaactagctgctaaatgtgccttgagtatcagaatgagctgataggcatatttatatttagaaaggaggatggctgtcaaagccttcagcttttgtgagaggctaaattatttttttaaatcataattaacaaataagtttaacttctccaaagttatattttagttaaagatttaaggctcctgatttaggattagctatgggcagaagacatgtttgcagtaaatctaagcaccatgacaaagatgaggccctcgacatgatcctgttttacataattttgaaataaaatcagaaggctgtttttattccacttatgacagtaatgctttcatttacttattcaataaacatttattgagttcctgttcattgtcctaggcactggaaattcagaagcaaattcttgaggagcttacagtttattcaatggaaacataaaatatagaaatgagcaaaacaagaaaatatcatatagtaataacctaccttgatgaaactaaagcatggtgatgtgagagtgatgtgtgggagcttcttttgattgactgttcagaaaaggtccctcaaggtgtcagtagggaattgtataatgagaaagaaccagtcatgcaaatatcggggaagaaaatatgctaaagactttgagaataatcaaacaggtcacacacaagggacctggaagctgaatggtagtggacttccccacatcaatgctggagagcagtggcacaattccttcaatattctgaatgaaaacaactgtggaattataaacctaggtaaactacctatctagtgagagggaataatggaactaagattctggaaatacacttccaggaataagaaggcagctgaagaaagaagagaaggtgcgtgccagggtgacagccctgcaccaggccttgagataatagttttgacgttaatttcttatggggactattttctcgctattttggttgaaggtatagtctcaaataaatagacatttaaacaaattgagcaacagactgcaattttcagttttgtttacattgcacaaaaaatcaaaagaaatgaaatttaagaaataagtaggtctggatattgctttaagagagtaagtattacaatataagtgatagtgattttaataataaaattaaaatactttctaatattttatattataaaaataatctaacctaggtgaccccattaaaaggattgaacatgataatataactcagggttcaatttgagttacaagtatggatttaagaattttgttgacaatcccggctgtcactgcttcaacaaagtttttccaaattgaaaataataaggaactctctaaaaattacagtgagttaagaaaggtggtctaattggcattactgtcaatagaatgtaaattatgtgaaaatctagattataaaagcataatttttgatcttgctgaaatgtagaaagaacaaagacatgttatagaatacttaaactgtatgacttaggaatgtctttattattcatccaagcaattctggctcattacagaatactcagctaagcaataatatttaaatttagaagtctctggtgttttatctgtgtttagtcatataaaaatcatagttttacacatatgtttttattttgttttcatggaagcatatttatcacagtgaaataatagaagatattaacatttaacagttagcttgattataacttttaaatatttagacgaaatgatttgagagttccttaatacctttgctccaggcctactaagtgtgaggcacaacagtctatgtggcatgcgtgtgggcacgtgcatgctttttggggaaaagtgggggtcacagaggggtaaaaaggaagatgtaggaaaaagaggggtgcacaagatgctatttccccagttattcagttattgttactaggtaatgtgtcacatgtcaccttgtgacatgctttgtgtttttctcttcgacgttcctgtgtagccaagtgtggagtcatttcactatctctgcagccgggtggggctccccagcccagacacagccttggtgtttgttggatgttgattgctgaggaatgtttggtactttggggagggtgtcagctcttcaaggccactagagggaccctggagctcgttcctccagcaatctgtcatcagtctctctttttatgaaatttcctgtgctttcatttaagaatagatactattaaatataaagtgatttttagaagttgattattgtgaattttatgtgtaaagttgaggtgcaataaaatagaatttagctgcaaagtgtggagatgttagggagattttctttgttgatgactgatatgaagtgacactctatgaatagaatgctaacatttttgaggtggttgggcctccatgagagtttaaatacacataattcattcaacaagtatttttccagtacttaacatatacttggcattattctaagtgctggcacatagggcacagtggagaggagatgaggacccagagcagttttcaagaagtaacagatgaggtgagacagcagagttcacagattccaaactttccacccttccataacataactgaaatattacacattttgcacagaaaaatggaaagcactctttttttttttttaagtatgaaatacaaaaaaaactgactggaagttctgtgtgtacatatatgcatggggcttgtctaccagtcttttgggtggtgtgtttttctccattcttgcctggcggtgggaagaagggtagaatattactggctatcacttgggaagtggagtgggaggagaaggggactggggcactaagaatatttgacataacacccctttctttagtactgtacttcccaactgtcttcagtttgctttgtgtctttgaattgagggtctctctagttcactttccccaggggtagatctccagtcatcatttggggtggctgccttgccacattggcttgggaggaaggacttgaggttctacttgttccctaaatagacttgcagccacttctcttatgtttagttccggtctttgccctctgccttcagaagtacttggtgcctctaatccctgaacttttttgaggttctgggtcctgcatcactttgcttcttgttgactttctttgtgaagtcagtcagaacttgccttcctctttccatcttccaaaatacaaatctcaatgctgtcatgttgtctctctttcctcctgtccttgcagttttatgtctttattcctttataaggacatagcttcaagagggcatttcaggatggactagagataaatatgtatatctggtctgcactatttcactaaaagaccatttactattttaaatcatatagttcttgaacatcacaagatttaaaaacagaaattagttaatttcccctataataacaattaattaaaatagaaaactatacaaataataaaatgacataagaaatgatttccactgatcttgattgactggtacttacataaaaacagtaactagaggcagatgatatagccaatcatgaggagaaattctgggttgtctttgctgttagtggaatttctctctcactgggtctatctcagagcatccctgccctggacaggtcattttatctggctttactctatgtgttgctaaggtcatgaaaattggtacctgaatccactgaatttagcctactttcactgatatgaagtatacttttattcatgaaaaactgttctaaagttttttttttattactccacaaaattcttatttcttattttcttactttaacaaggagactgagatgatgatttggaatgtgtgaactctgctgtctcacctcattacttctgttacttcttaaaaaaactactctgggtcctcatcttctctccactgtcccctttgtgccagaactggtgtcctttctcctttttaaggccagttcctctaaatattctcatctttcctgtggcctcttccagttttgttttgttttgttgttttaggacagggtctcactccataacccaggctggagtgcagggcttaatcagagctcactgaaatctcaaactccttagcctcaagggatcctcccacctcagcctcccaagtagctgagactgcaggcacatgccaccatgcctggctaactaaattttttttgtgtagaaatggggtcttgctatgttgcccaggctgatcttgaactccaggtctcaagtgatcttactgccttagcctcccaaagtattgggattacaggtgtgagccaccacatctggcctgttttcatttatctttcctgaatctctccttttcccgtgtctacaaacaagttccatcttttctacctcaaggtgttatttttttcctcaatctgctactcccaagttttctcaaaccttctttcccacaactgtccaacttttctaaagattggctaattctgaaataagactatatatgttagacatattcttaaatgctttacatatattaactcgtttaatcttcacagtaacaaccctgtgaggcaagtacgattattatttccatcttgcagatttggaaactgatgtacagagaaggtaaggaacttacccaaggtcacacaggtaccacattaggattgaagtgcaggaatcctgtttcctggtctgtttggttaacttaccttacactacgctgcctattgtttccatttgttaaccagccttcctcatcatctccgtacatgcttaatcatgtacagtttctaaactcactacctattctctgaacatttttagaataatgtaagtggttgttctaggcagcatgtgatattcctctttttaaaattaaattaaattattcattttttttgagacagggtcattgtaacctcagacacctgggctcaagcaatgctcccactccgccttcagagtaattaggactacaggcgtgggccacaagctatgttcctaatcctcattcactctttcttcctgagccattcctatagtttaacacgtaagtgagtacctattgtgggctaaatgctagaaatataaaggcaggtttttcgctggagcccctcccctccctgttgagttacttatgaggtggtcttggtaacatgataccatccttgctctgtcaccattttagctgctgtaatactttcctggtgcttcttctgagtctccttttcttgcaaggcagttggatgtggaagagaagcataggctttggaattacacaggccattgtttgcatcttaggattaaaatatgttgtttgatttgggaaaaaataattaacacctttgagcctttgctcatctgttaaatgaggagcaaagattcttaatttactgatttttgtgaaagtgaaataatttcttatgggtcaactatgcaacattgtgtctaatacattgtacacttgtgataaatatacatctccctccctcttccatttaccatttttgtcttttcttagtttttatcatagtcatcaagtcgcatcacttcatcttttgcacctttgggaatagcttctaaatccatatgcttaattatgtaaagatcatcacttaaaaagatgacttcttgtaataaaccacatagctgtttgaatctatttgtatatgttccttctaggttagggtaacaaaatgagaaggtaaaatatacagaattccattctttctcatttttcttatctaccttaaaatttgagtagattttctagaaagtaaagctggactttaaaagctcataggtaatttagtaataaagacaaccttctgaatataaatgcccacaagcctgcctttgacctgtcagataaattaagagaatttggaacatttactctctcttaatggaagagaatcttaaagtccaatataatttgctattcaaatgacgatttatcaggctttgcaccaccgattctctgttagagccttgaaataccagctagagccacttgccaaggaaggaaaacactacattaagaactacttgcttttccccagataaaagcttgtccaagttatgtaaatttgacaaaagcattttatttgatagctaatgtcaggagggatctagggttatctgttctggttccttatgttagctaatcatttggccagctagtatattccattcttgtggaggctggtggcatcaacatttacacagaaagttcattaaaaaataaaacaaacattgggccaggcactgttggctcatgcctgtaattccagcactttgagaggccaaggcaggtggatcacttgagtccaggagctgaagaccagagtccaggagttgaagaccagcctgggcaacagtgagaccctatcttaaacaaacaaacaaaatgttgatattgccaactcttaggtcagctagtataatcaaataagattaggtatgaatgtgcttttaaaggtacaaaaatctgtactatttactctgtttggagaacagtcagtttcttatgggtgtcagtcatggactactttgatatccatatgaaaagataacattctcattaattgtgtgataaacatttaaatagtcagcatttttgtggagtttataattttaatcctttggaatgtgctttcaggtccatgcaaacacaattccaatgttatgaagttttgagttctggagataatctgtagacttttaggtttttcttttccctctgtgaatagttttttatatacatatatataatatatatatttacatatatacacatataaacacatatataaagttttatattatatattaatatgtatcatatacatatatattattatctaataaatagatgtgtaaaaatgggtcatggagtatgcacacatttaattttgctgaataacaacaaaatgctttccagcctcttatgtttgtttaccttctcatgaacagcaaaaaataattcctgttttctctttgtctttagcaatacttggaaatttcagacttaaaaagattattgccaatttaataagtctgaagtggtatctttttctgatttgtctttctgattatagtgcagttgagcatcttttcatatgcgttttggtcataagtttacttttatgtttcctattcatctgttttgcacatttttagttgagtttttttttcttaatgatttgtgttaaagaaaaaactattctgacacttgttaaaacagtaaggaagagtgtattcaagactgttgcaataagggttgcttctatggcaacagcggagagagattgggttcaactctaaataaagtaaagacaggttggcatttatagccaattagcagagagaggggaagaatgcatggaatattcttaagagaaaacatcaagggtagggggattcctactaaaccaatttaatggaattcttgctgaaggcaggccagggtgattatatatcactttggggatggtggagaatgaagaattttttaaaaataattatttgatttttccctgagtcagagggaatgaggaatttgatcagatattaagagtgatcatatataaaggatgaggggattcttgctaaactgacttggcaggattcttgctatgactgaattctgcaaaaatggacacagaagtcgaaggtctggttgagaagagacctcagaggagcctaactgaaatttggtctaagaaagagtctgtcatttgtaggagttttacatatattctggatgttaattttccttgattaaatgatttataaatgtattctccaaatctgtggtttctcttttcactaagatttccttccatattttctttcaaaaattttaagtttaatttatttttgtatatgaggatccagttttttcctgtataaataagcagttatcctcgcttcttttgctgaacagatcattttattcctgccgtgtatcaagtttgtatgtattaatgtatctgtattttggctttctagtctctttcacttgtctatttggaaaaaaaaaacaaaactctgacctaccaccacactgccataattattatagtttttagtcagctataaagacaaggtctgctcctcaccttgttttttcactaaaagttgtctttgtttttccatatcaatatcattgtggagattttcattgacattgcattgaatttagagattaactaggctgcagcttacatttttatgatatttaagattttttggtttggtaatttactatattagaaatatttttaaaatattctgttagatttcttcctaattactttgtagctccacaggtatcacaaatgataacttttttcctactgttttctagttgattattactgctatgtaagagtacactgattttagtacactggttttgcgtcctgcagtagtgctgaattctcccagtattgttttgtttgtttattcttttggactttctgcatccatattgaagagcaaagaagccctttttaatttgttttctaattgttttaactcttatttctcttgttttattggcaagggctcgtaggacaatgttgagtagaagtggtgatagttggtaatttgtcttgtttccaactttaataggaatgcttctaagctcagtaggttgctttctttagttttctttttcttttaaatagccttaattagattaagaaacatttttctcagtgttgaatttcattgcaacttttttctttttctatattacagctaaatataggtaatctgtacttggctgatttttctacttttagaattagggaggcttaaagcagagtgaaaaattaaatgagtgaataattatattttctcacaaaatttgtgtcatttttgaattatacattgttcattaggtgttaattagtaacatgaacattagtgcaacttatctttgtcaaaagtacttgagtcaggtagatttgagtttaaatctgactctgataactaatcgggtttcttcaacttctaaaactcactttcttcatctgtaaactggcaataatatctacaaggcaggattattatgaggattaaatgaaatatttttaaagttatgactaagagtaggtatgcattaaatactaaatcttattatttctgtgtctaccatacaaccatacatcaccatcatgctcctgagaagtgagggtgatggggattaagtaagagaaacagcatctaacattatgtatagatgcaaagccacaaagtcctctatttcaatgacggaatattgagcaactgttgaaagagtaggagacttggttcaaagtcatcgaatcatatgccagatttataagcacttatcaactggaaggaggaaagcattttatgcagtggggtttcttgaagacataatggggatcattttgaagattcaaaagtagatggattagttcatgtaagagcaatttatagccaagagtgatatatatgagggcttccagaatagactaagagaacagggcctccatggcagtttgcagagaacaaaatcaaatagcaagtttgagcaaaggctataaaggcagttggattgaatttttagaagttaattaattaaattgacaaataataattgcatatatttatagggtataatgttatgttttaatatatgtacacattgtagaaagatgaaattaaacttattaacatattcatcacttcacctatttttcatttgttttgttgtgaaagcatgtaaagtctacttttttaaaagcagttttgaactatacattatgctatcattaactatggtcactatgctgtgtaatagagctctaaatcttatttttcctttctaagtataactctgtcccttttgaccaacatctctcctcctcccagtccccaggcctctggtatcaaacattctactttgtttttatgagctcaacttttttagattccatgtgaaaatgaaatcatgcagtatttatttctgtgcctggcttagtatcctcccaggttttaaaatatgcagcctttaaaaagaaggaaatgacaggatttccttctttttaaaggctgcatagtattctcttgtgtgttgataccacattttctttgtccatttgtctgttgatggattgaattttgatgctttgcttcttcctgagaacacatttcctggtgtcaggttcattagaagagagcttgatggacaaagtcccatgtgctaggccatcccatccttaggggcataacaaagaaaaaacagaagaaacagaagaaaatgaccaggggccagagtggaatggagtggtggtatatttttagctgtgaatacaggaacccctgtggaccctagtatattggaccttctttgagaaggtgtgtctttgtttgcattgccctggctcagacgaatgagaataatagatcatcttccatagtatgtttatctgaagatagagaactggtgtggggtagggagagaacattttggaataattttcaagaaagaggagattgggaagatctgccatggacagcagaaactctgtgggtaaggtgagaggtccaaagtgtatttaggacgtgttgctgtgtggtgtgtgtgtgtgtgctgaataactcattccttggtttagaaaaggctttggcaaagaatgcatacattttcaatgcttctaaagttacatgtagctactctggggaacattattttgctgcaagtagctgaaagaagaggaaaggctggatctggtaaagagatataggaaaagactagaaaagtaaactattttaaggagtttgtcattgaagtgaaaaaaaaatgagggagaagagcagttcagtggagtatcaggatttgggattgtgttagatttaagacagagaggaaaggccaggcagacagggagaggttgaacgttcaaaagagaatggaagtcatggtagggatatcttatggagcacatttctagagaaaaaatgaaaatagagtacaagtggagctgttcacaatgaaaaggaaaagggttcttattctaagatgggatggaaggtggagaaagtgtttgtgggtggattgtgagttagatgttctcaatttcaggcaggtgggaagtagtaatgaaggactgtggggttggggaaaagtgggaaaggcttttaataggctctctcttaatgtggttcacgttgactagcgatgataatacattgctgagtagaaagtcagaatcctcagccctgagccagtgtcctcattttcaggggccaaattgttgacgtttaccttcttcacacctacaaactcaaccatgtactacacagctgtgagatacagagtggtggcccatctagttagaatcacacactgttgtagacaaggagaacagtatgccttgaaactctggagtgcactgggagccaggtgatcacatgaaagtagtctgcttctttgtatgaagttccagaaacatcattatgttctttacctcctggaaggctgtccctgctcagatattctctgaaccatagtcggtgtcaaaatcagatgaatttggttccagaccactgcaataaagtaaatatcacaataaagcaggtcacatgatttctggtttcccagtgaatataaaagatatgttttcactacactatagtctattaagtgtgcactgtcattatgtctaaacaatgacattgatatcttaatttaaaaataccttattgctaaaaagtgctactgatcatccaagctttcagcaagttctaatgtttttgtaagtggagggccgtgcctctgatgttgacggctgctgaccagggtggtggttgctgaagtttggagtgactgtggaagtattttaaaacaatgaagtttgcaacatcgattgcttctttcatgaaagatttctctgtacatgcgagaacatgctgtttgacagcattttacccacagtagaacttctttccacattggatgccatcctctgaacctctaccactgctttgtcaactacatttatgaagtattctaaatcctttgttgtcatttcaacgttgttcacagcatctataccagcagtagattctgtttcaagaaaccactttgtttgctcattcatgagaagcaactgctcatttattcaagtttgatcatgagattgcagcaattcacttccatcttcaagcttaacttctcattgtaattctcttgctatttcccgtacacctaccgtgacttcctttactggattcttgaacacctcaaagttatccatgaaggttggaatcaacttcttcccaactcctgtaaatgtggatattttgacctcctcccatgaatcataaatgttcttaatggcatctagattgatgaatcctttctagaaggttttcaacttcctttgctgagattcattataggaatcactactatggcagctacagtcttacacaatgtatttcttaaataatatgacttgtaagtcaaaactactccttgacccatggacttcaggagggatgttatgttggcaggcatgaaaacgacattcatctccttgtacatccccgtaagagctcttaggtgactagatgcactgtcaataagcagtaatattttgaaaggaatttcttttatttttctgagctgtaggtcccaacagtggactgaaaatattcagtaaaccatgctgtaaacagatgtgttaccatccaggctgtgttgtttcatttctagagcacagacagagtaaatttagcacaattattaagggccctagaagttttggaatggtaaatgatcattggtctcaacttaaagttaccagctgcattaaccccaaacaagagagtcagcctgtcctttgaagctttgaaaccaggcactgacttcttctctttagctatgaaagccctggatggcatcttcttccaacagaaggctgttttaccaacattgaaaatttggtgtttagtgtgccaccttcatcaatgattttagttagatctcctggataacttctccatcagaacttgctacttcacttggcacttctatgtcatagagatggcttcttttcttaaacccaatgaaccaattctgctaccttccaacctttcttcttcagctttgtcacctctctcagccttcatagaattgaaaagaggggcttgctcctactttagggtatgttgtgggtggtttgatcttctatccaaaccactcaaattttactcatgtcagcaataatgctgctttgctttcctatcatttgtgtgttcattggagtagcacttttaatttccttcaagaagttctcctttgcattcacagcttggctgtttggcacacaaaacctagatttggtctatcttggcttttgacatgttttcctcactaagtttaatcatttctatcttttgattttaagtgagagacatgcaattctccttttacctgatcacatagaggccattttaggattattagttagcataatttcaattctcttgtgtctcagggaatagagaggccctacagaagggaggaaattgggggaatggctggtcaccggagcagtcagaacacatacaacattaattggttaagtctgccatcttatgggggcacagtcatggtgtcccaaaacaatgacaatagtaacatcaaaatcactgattacagatcaccataatagatataataataatgaaaaagtttgaaatattgtgagaattgccaaaatgtggcacagaggcatgaaatgagcacatgctgatagaaaaatggtgctgatagacttgctcaatgcagggttgcaacaaaccttcaatttgtaaaaatcacaatatctgagaagcacaataaagtgaagtgcaggaaaaagaggtatgcctgtacctgtcactcctgaataactaatagaaataataatggtggtagttggtggtgtgatgctggtggtacctgtcacgtgcaagaaatatacccatactttgtaaatattattcatatgcaacattcatttaggtgtaattatcttgattgtacagataggaaagctgaatttcagggaggtttaacatcagagtaacgatttgaacttagatttgttacactcagagttcttttaaaaaagcaaaattacttcctgatttgtatgcttctcatcgattaaaatggctactttaaataaaatctaagtatacatttatttttgtgaaaaaattcatatttatttgactagaaatttttgagcatataaaactagaacttactgacataaaaaatagttattgtgtagtgtgatgagtactagagtagaggcatatgcaagttactatgttgagagagacgtggtgtgttttggggtgggacgtgatgcaaatggagtggcgcatattgggatggaacatgatgaaagcgaaatggtcaaggatgctcttctgagaaaatcatgcttgtatccatcttgacagatatgtaagactttactgggataatacagttgggcagaacagcaacttaagtgcagagagtgcttccagtcattgtttttaaacatttgtcattttctaattttgaaacattcaagaaagtggctatagaacaatggtttttaatcattcccttagacagccgcttgatgaagcccattacctgttgcaacagacctaccaaattttgcatacaatttaaaggggttcatgaactcactgatgctcctctatgacctgccagccaatactctctgatatagagaaatacttgaaaaaggaacataaatggctatgggtaagagtagagggcactggggtagctttttagctattattatattaaattcctacaatagcactatgaacttggtattaagtcaacttacagctgcataaattaagatccagttaagtacagtgactccctcaagttcacacagataataaacagcaaaactatgctgaggtccccttctggggtcttgcatcccaagctcatgtttgctccatatacatgatactaatttcctaatgagaatattgctgtaataagttatgatttctaacaattagcttacaaatgaacttttgtaaaacaacttgtagtgagttgaggattgcccgcatagtgaaaccttgttagttttgattaccagtctgtcatagctgataatttatgatttgttttattttgctatgggcttggctatagagtattttggcagcatctgtaaaaatttcttttgaaacaatttttttggcaagattgcacctaaagcttctcaattatctagagaaattccaagttgttattatgtgtttgatatgttgtgttagtcttatccatacactgtggcaggttttgtgaagaacctctaccgttatctcgcctttgatgctggagagcccagtttgttagtgttaagttctggaaagcaataaaagtgcatctatctaaacatgttctatactaaattgctttcactaatcaagcaggacttatccctatttagaagaattagtgaggttttagtcaattaggcttgatgtcaagttaatttttcctccattagtgtaatgaagaattgctgtgttgttctgtcatgcatgctgattgataaaaaaagttaataatgaatgccaatacatttttctcttgaaaaatgaaccaataatatatgaggttcaagtaaattattctgctcatcagaaagaattgcttattctactgtgtggaaagcccagtggcaaaccaggggaagcaatggaatatttaaatacttattctattgctggtcagtctttaacatcagctttttaaaagattgccttataaaggatgtccatttcataattgttggatgactaaaatagaccactttgttttctttggcttctgctatttgcttatttaaaaatatttatattgtgctctattctttgcatgctgcattctgcagtttgggaatactgaatcaagacctggtgagtaacttttactcagtggattgcatgatgaaagatttgccaaaccataaggtgttttattgtaataaatgaaatccatctcccttgaagaagcacttgcagaggcagaatttactctacattttattattccattttatttattctatatattcaaagtaaggattctaaatcaaaaagataccaaagctaatgcatcctgtaaatgaagtgtggtcttgccaaactgctttttgaggttgacattactatattatatagtaattagcagactggctcatgcacacatacacacatgcacacacacacacacacaccctcatctataatatttatttgtctctacaatcatatctattcagttttgtgtctgaggtaaatttccagataacaaatttcacttttataactattaatacttcactaataatcttagtgaataaaaaaagaattgaagatatttgaagaacaatttaagtgctgactcttaataaaagtattaagggttagagttagggcattgattaggtagtgaaatttgattctactatatgtctgcttcttagatagagaatttcagtcccattctaaggctctcaattcaattaataactcttcatttatttaaattaagtggtgacattctcctgcctttaactttgctctccatgctaattattggctcaatttatttagaagtattattatagaagttttgaatcctaataagtagaaagagagaccaatgtgtgagcctaattctttttcgaagtgattaaggtattattactttttatttgttatcaggtatcagaacattaaaagtaaaataaaagaacagactttattttactgaatctctttctacttaggaatatcaaagtgcttaatgaacatgactttatcgaacctttataccaacttagtgaaagtggataaaagtcagaaacgtttagcagcagaggaaacaccaatagagattacattggagggttccataagacattccggcctctggtcttatgctttctagtgtatcctcctctattaccaggttacaaaatatatattggaaaaaataagtgagggatcagccatcattatagtttcctgggttgcaaacaaacacccaaatctagagattatttccatttagaaagtagaatctcaaacattcttttctggacaaacatcacttttgcaaaaatagtctgtattttttttgtaatttttatagtgtttgtgactttgatcaaaatcccagaatcatgtaacttttatattaaaacaataatggcaatcttaaactgggcctctgtgatatatcaggctacttttgtgacaacattttatattctgaatgatatcctggtccatacattaaatcagtgatgctatattaaagcgtaggaagatggtattcagtcattcatttcatcatttattttcttctcatctattcagtgatcacctgccaagtgccaagctctgctctacaccttagaaattccatgacaaactagacagaaaatgttcatgcgctctgccattctggttcattctagtgggacacacagcccaacatcatagagttacaaatgccatgcaggcacttataagtaggagctaactaatgtgtacatgtggacatagaatatggaatgatagacaatggagattcagaagggtgagcaggagggaagagggtgcatgataagatattacttaatgggtacaatatactttatttgggtgatggataccctgacatcaccactactcaatctatacatgtaacaaaattatagttgtaccccatacatttatacacataaaatacaaaaataaattttaaaatgtcctgcagggaattaaaatgtgatatgatgatgtctgggtcactatgttagtgtgggtggtcagggatgacttcctcaaggagaacacatttaagttaaaatcagaattgtaagaaggaaccatccatgtgaatatccgagagaataatattatggctgaggaaacagccagtgagaaatctctatggtaggaaacagttcagtgggttttaggaacagaaagatgaacagagtaggtaggcacagtggatgcttgtataaaatgacattggaaaacattctagatagaaattcttcttcaaagtaagcattcctgtgttctaacacttaaacagtgttaataatacaagacattggctccaaaattacattttattaaatgttataaactaagaacgggtcagtcaggaaaacaactgtcttttgcaatcagtttttgcaatctgtaaacagtagattatatgtagtggatttgaaagctctaggatggtgttttttttctcctcaacaattggaagtattattttaattgtgtgtgaagtgttagcaatcctcagaacacctcagcacattagttcagtgtcttctactcaaaaccagaggtacttgaatcctggaggtactccaccttccaggatggagactggcaaacagcaataaaataataatttcttaccttatagcaacaagctacttaattcagaaattcctcatctgaaaaactcaattctcttgagcagtatcttgacctcttattgcataggcttaggagtcaaatagtctgggcacaaatcctgactttcccattttctagctgtgtcgttttgggcaagtcccttcttccagtgtcctcatttatttcctctgtaatcaggatgatgtacttatcacaggcatttagaaaagcatcttgcaaacaataaatctttaacaggttatctgctttcattgctaatattcttacttttatgccctccctttccaaaccatcttctgaaaaaatatgtaagtccacaaagttagatgcaagatggttgccaataaaagtcttctgccttgagggctgggtgtggtggctcacgcctgtaatcccagcactttgggaggctgaggcaggtggatcatgaggtcaagagatcgagaccatcccggctaacacggtgaaaccctgtctctactaaaaatacaaaaaattagccaggcatggtggcacgcacctgtagtcccagctacttgggaggctgaggcaagagaatcgcttgaaccggggaggcagaaattgcagtgagccaagattgcgtcactgccttccagcctggacgacagagtgggactccgtctcaaaacaaacaaacaaacaaacaaaaaactgccttgatcactagaatagaggaggaagcctctgagatccattgactgaaatgtattttgatatttcatttctctacttaattcagcacccccccccaccatttaattaaaaataatatttattgaacacccattaaacgctaaattctcggccaaaaaggaaaaaaacatcttgtcctgccaggagattttaatccagttcttcaactctcagtagctaagaattggcaaacaaacaacaaaagcatctctgcactgaattttatgttatattggtgaaccaatttggaattgattaatgaactttttttatgcacatcctttccatgttgtgcataaatgtccatgtgtttgctctttggaaagactggaatacatgcttcatgggacaatgatttgtgaacatgtcatgagagaagatgtacttttaatattactacacatttatttagtcaacaaacactcactgagtgcctacagtgctactacattctgggtgaacaggaaccaacaacacagaaaataaaatcctgtatctcctggggatcacaggcttagcttctttggcatggaggctcaaatcctgtttctgcacatttctagaggtatgactttggttaatttacctaacctttctatacctcagttccctcatttgtaaaatggggataatatctatatagggcagttgtgaagattaaacgatggtatatgtaaaatgcgtaggacagtaccaggagcatagtgagaatgcggcaggaggctctgttattactataaatattattatcactactgctctgtgctccagtggtgtattctgtatatttcatgcccgattcttcttttcttaaacgggtcagcacatctttagagtggcacagtgacagctttcagagggaatgtggtcctggtacccttatcttgtgtggctcctacttctcacgctccatgtacagagtagagacagcctctgtttttctgaactattgaaatataaaccagggtgccagttgccccttgcagagaatagctttatatttgtctctctgttggcaccttgcctattccttatcacccccacttggtcctaagtgcctgttctgtcatggtggttgcagttaccactggctctaatctctcttctcatctatttctttgcctcttaccactttccctaagctccttgaccgtgtgtttctagatcttctctgccacagaaagcagtctctctttttttgcaggtgagtcacatggagtatattgagtgggatcttgaatatttcagttcttgatgtaattcatgtcaaatgttaagacatgacaggcagctgctgcacggggttggtttctccacggcatcccatcctacccctgttttctcatatgaattattctgaaagccttgaggctgatcagtgtagatattcctatattttggaaacactacttttaaggatcctagcaactgatttctccaacttacattgaaatttaaacaatttcctggtgcctcctgaatccctttaagatgttgcagtaagaatgagagttgtgtaggcggggaggtacattttatgggctttttataagaaatgatatcctgtgtcctgaatgtaccagtgcattaatgccatcctcatctgtagcaacagtaagcagccttttagagtggtataaatagtccattaggataaccataaatatttaactattatttactttagattccattggcaaagtagcagatttcttgaaaacctctcaggtttctcctaaattgttcttttcaaggcagtaaaaatgctatggtcccaaactgaaaataactgattattaattaaactactaaagaccaaaacaactttactcacattccacaggattggagagacaggaaacaacttgcgaggtttctagaaattgttattttcttgttaatggttattttaattagcaaagagagttttggaaatagttttgagctttacctgaaactgaatgactggaaggtaagtgcatttgaattatatttggttacatgcattaaaggataaataatttctcttttctcatgtgaagcttcattttggtagttagctgacttttcaggtaaggagaaggaccaagtaatcaccagctataaaatacaaacatttatcactggaaaacctagtaatgaaagcaaagcctatcaaaactttatgtttacagaatgcgtttaactctttttaatatattcatgtctatgtaatccttttgttacttttttcatgaaaacttatttaacagtatatatagttcatagtattgtgttgtggaatgagcattagactttggatatgtcaaatcctttccagagagagatggcctactcaaataaggataaataataaataggggagagacactttgtaaaactctgagcacagtgtaggtgaaccacagggcagtaacctgaggctagtaacagtggagctgtcaacaacctatgaccaaagggaaaagggaagaaaaaaggttctctagaacttgaaaggagctagttttgtgtagaaagctactgtacgtggaacagtgatctttagttgtggacatagccagcctaagttaaccttcaaggagggagccaagggaataaatatcccgatccccactcctccttccctccaattttgttagagctcactgtctggaacctgctgagcacctggacatgagggcctttgatagagtgcctgcaggtcttatggggaaagaagagcggaaagcggatctggagggagaaatgaaagataccttgaatactctaccacttttctctctatcaacctccaatcttgtcctccatgtggttgacaaatgcatgttcctaacacagggtatgcacaaaatcccatcaactaccatatcattcccatgatgtcttttactaatactgcaacctgactaaaatattggtccccagcaacactcttcatataaaatattgggagaagaggatgcaattaaggaacattaattatagctgcagctcacttctggagctggtcatgaggcctaaactgctcattatagggcccccccatcgacaataccttctgtatttaccttactgtctgacatcaccttgactggatgagtcattttccagttagggcaattgaaatcttcatttctttaagatctgagtgcttggtagtcttgtttttattgggggatcgttgctattttcccttgaccaggactatcgggaagggagtactaagaggcatcccactgaattccctgagttttaagcactgtccttgtcttcattctgtagcagaaaatcaattccccctgatcattaggtgtgattaccctagctagttatagtgactttctcatctctgttggttcagggccataaataaatttaaattgccaggagttagtctctacttccagtgaatcagaaaatgaccatgtttcctggtataagaattactgccttggacagtagggccatcaaactcatcaagtccaaggtttcggggacaggaggcaagaattctttttttttttattattatacttaaagttttgggatacatgtgcagaacgtgtaggtttgttatataggtatacatgtgccatggtgatttgctgcatccatcaacccgtcatctacattagttatttctcctaatgctatccctccccttgccccccaccccactaacaggccccagtgtgtgatgtttccctccctgtgcccgtaagttctcattgttcaactctcacttatgagtgagaacatgcggtgtttggttttctgttcctgtgttaatttgctgagaatgagaatgatggtttccagcttgatccatgtccctgcaaaggacatgaactcatccttttttatgtctgcatagtattccgtggtgtatacatgccacattttctttatccattctatcattgatgggcatttgggttggttccaagtctttgctattgtaaatagtgcagcagtaaacatacatgtgcatgtgtctttatagtagaatgatttataatcctttgggtatatacccagtaataggattgctgggtcaaatggcatttctggttctagatccttgaggaattgctacactgtcttccacaatggttgaactaatttatactcccaccaacagtgtaaaagtgttcctatttctccacagccttgccagcatctgttgtttcctgacattttagtgatcgacattctaactggcatgagatggtatcttattgtggttttgatttgcatttcgctaatgatcagtggatgatgagccttttttcatatgtttgttggccacataaatgtcttcttttgaaaagtgtctgttcatattcttcacccactttttgatggggctgtttgtttttttcttctaaatttgtttaagttccttgtagattctggatattagccctttgtcagatggataagattgcaaaaattttctcccattctgtaggttgcctgttcactctgatgatagtttcttttgctgtgcagaagctctttagtttaattagatcccatttgtcaattttggcttttgttgccattgctttttgtgttttagtcatgaagtctttgcccatacctatgtcttgaatggtattgcctaggtttccttctagggtttctagggttttaggtcttacatttaaatcttcaatccatcttgagttaatttttgtataaagtgtaaggaaggggtccagtttcagttttctgcatatggctagccagttttcccaaaaccatttattaaatagggaattctttccccattgcttgtttttgtcaggtttgttgaagatcagatggctgtagatgtgtggtgttatttctgaggcctctgttctattctattggtctatatatctgttttggtaccagtactatgctgtcttggttactgtagccttgtaggatagtttgaagtcaggtagcgtgatgcctccaactttgttctttttgcttaggcttgtcttggctatacaggctcttttttggttctataagaaatttaaagtagtttttttctaattctttgaagaaagtcaatggtagcttgttggaaatagcattgaatctataaatttggactgtatggccattttcatgatattgattctttttatccatgagcatggaatgtttttcatttgtttgcgtcctctcttattttgttgagcagtggtttgtagttctccttgaagaggtccttcacatcccttgtaagttgtattcctaggtattttattctcttggtagcaattgtgaatgaaagtttgctcatgatttggctttctgtttgtctgttattggtgtataggaatgcttgtgattttagcacattgattttatatcttgagactttgctgaagttgcttagcagcttaaggagttttggggctgagatgatggggttttctaaatatataatcatgtcatctgcaaacagagataatttgactacctcttttcctatttgaataccccttatttctttctcttgcctgattgccctggccagaacttccaatactatgttgaataggagtggtgagagagggcatccttgtcttgtgctggttttcaaagggactgcttccagcttttgcccattgactatgaagtgggctgtgggtttgtcataaatagttcttattattgtgagatatgttccatcaatacctagtttattgagtgtttttagcttgaaagggtgttgaattttattgaaggccttttctgcatctatcgagataatcatgtgttttttgtcattggttctgtttatgtgacggattgtgtttattggtttgcatatgttgaaccatcctcgcatcccagggatgaagcctacttgatcatattggataagctttttaatgtgctactggatttggtataccagtattttattgaggatttttgcgtcgatgttcatcagggatattaggctgaaattttctttttttgttttgtctctacctggttttggtatcaggatgatgctggcctcataaaatgagctagggaagagtccctctttttctattgtttggaatagtttcagaaggaatggtaccagcttctctttgtacatctggtagaattcgactgtgaatctctctagtcctgggctttttttggttgttaagactattaattactgcttcaatttcaggacttgttattggccttttcagggtttcgacttcttcctggtttagtcttgggatgctgtatgtgtccaggaatttatccatttcttgtagattttctagtttatttgcgtagaagtgtttacagtattctctgatggtagtttgtattcctgtggatcagtggtgatctcccctttaccattttttttattgtgtctatttgattcttctctcttttcttctttattagtctggctagcagtctacctattttgctaatcttttcaaaaaaccagctcctggattcattgattttttgaagggtttttcatgtctcaatctccttcagttctgctctgatcttagttatttcttgccttcagctggcttttgaatttgtttgctcttgcttctctagttcttttaattgtgatattagggtgtcaattttagatcttttccactttctcctgtggatatttaatgctataagtttccctctaaacactgctttagctgtgtcctggagattctggtacgttgtgtctttgttctcattggtttcaaagaacttatttctttctgccttaatttcgttatttacccagtagtcattcaggagcagtttgttccgtttccatgtagttgtgtggttttgagtgagtttcttaatcctgagttctaatttgattgcactatggtctgagagactgtttgttatgatttccgttcttctgcatttgctgaggagtgttttacttccaattatgtggtctcttttagaataagtgctatgtggtgctgagaagaatgtatattctgttgatttgggggtggagagttctgtagatatctattaggtcagcttggtccagagctgagttcaagtcctgaatatccttgttaattttctgtctcattgatttctctaatattgaaagtggggtggtaaattctcccactattattgtgtgggagtctaagtctctttataggtctctaagaacttgctttatgaatctgggtgctcctgtattgggtgcatatatatatttagtatagttagctcttcttgctgcattgatccctttaccattatgtaatgcccttttttgtcttttttgatctttgttggtttaaagtctgttttatcagagactaggattgcaacccctgcttttttgttttgtttttgtttttgtttttgttttttttgctttccatttgcttggtaaatattcctccatccctttattttgagcctatgtgtgtcttcttacatgagatgggtctcctgaatacagcacactgatcggtcttgactctttatccaatttgccagtctgtgccttttaattgggccatttaacccatttacatataagcttaatattgttatgtgtgaatttgatcctgtcattatgatgctagctgtttattttgcccattagttgatgcagtttcttcatagtgtcaatggtctttacattttgctttgtttttgccatggctggtactggttgttcctttccatatttactgcttccttcagagctcttgtaaggcaggcctggtggtgacaaaatccctcagcatttgattgtctgtaaaggattttgtttctccttcatttatcaagcttagtttggctgaatatgaaattctgggttgaaaattcttttatttaagaatgttgaatattggcccatactctcttctggcttgtagagtttctgtagagagagccgctgttagtctgatgggcttccctttttaggtaacatgacctttccctctggctgcacttaacattttttcctgcattccaaccttggtgaatctgatgattatgtgtcttggagttgctcttctcaaggagtatcttagtggtgttctctgtatttcctgaatttgaattttggcctatcttgctaggttgaggaagttctcctggataatatcctgaagtgtgttttccaacttggttccattctccccatcactttcaggtacaccaatcaaacgtaggtttggtcttttcacatagtcccatatttcttggattctttgcttgttcctcttcattgttttttctctaatcttgtcttcatgctttattttattaagttgatcttcaatttctgatatactttcttctgcttcatagatttggctactgatacttgtgtatgcttcacgaagttcttgtgctgtgtttttcgactttatcagatcatttatgttcttctctaaactgactattctagttagcaattcctctaacttttttttcaaggttcttaggttccttacattgagttagaacatgctcttttagctcagaggagtttgtttttacccaccttctgaagcctacttctgtcaatttgtcaaacttattctccgtctgattttgttcccttgctggtgaggagttgtgatcctttggaggagaagaagcattctggtttttggaatttttagcctttttgtgctggttttccctcatcttggtgactttatctacctttggtctttgctgttggtgaccttcagatggagtttttgagtggccgttctttttgttgatgttgatgctattgctttctgtttgttggttttccttctaacagtcagacccctcttctgcaggtcttctggagtttgctgggggtccactccatcccctgtttgcctgggtatcaccagcattggctgtagaacagcaaagattgctgtctgctccttcctctggaagcttcgtcccagatgggcacccatcagatgccagttggagcccttctgtatgaagtgtctgttgagccctgctgggaggtgtctcccagtcaggaggcacaggggtcagggacccacttgaggaggcagtctgtcccttagcagagctcgagcactatgctgggagatctgctgctcttttcagagctggcaggcagtaacgtttaagtcggcggaagctgcgcccacagctaccccttcccgtgggtgcactgacccagggagatgggagttttatctataagcttctcactggggcggctgcctttctgccagagatgccttgcccagagaggaggaatctagagaggcagtctggctacagtggctttgccgagctgtggtgggctccgctcagtttgaacttcctggaggctttgtttacattatgaagggaaaactgcctactcaagcctcagtaatggcagatgcccctccccacaccaagctcgagtgccccaggttgacttcagactgctgtgctggcagcgagaatttcaagccagtgggtcttagctttccaggctccatggaggtgggacccgctgagcaagaccacttggctccctggcttcagccccctttccaggggagtgaatgattctgtcttgctggtgttccaggcaccactggggtatggaaaaaaactcctgtagctcgcttggtgtctgcccaaacaaccacccagttttgtggttgaaacccagggcccttgaggtgtaggcaccctagggaatctcctggtctgtaggttgcaaagaccttgggaaaagcatagtatctgggctggagagcactgtccctcatggcatggctcctcatggcctcctttggctccaggagggaggtccccggctccttgcacttcctgggtcctcaccctgcttctgcttgccctcagagggctgcacccactgtctaaccatcctgaatagatgaactgggtacctcagttggaaatacagaaatcacctaccttctgcgttggtctggctaggagctacagaccagagctgttcctacttggccatctggcctgtcagtaagaattctttaagtggtgtattaggcataaaaataagatagattactttatttatctgtttatttatttagagacaaggtctaactctgtcgcccagctggagtgcagtggcgcaatcatagctcactgtaactctccttcagcctttcgagtagctgggaacataagctcatgacaccatacctgggtaattttttctttctttcttttttttttctgagatggagtctcactctgtcacccaggctcggctcactgcgatctcggctcactgcaagctctgtctcccgggttcacaccatcctcctgcctcagcctcctgagtagctgggactacaggcgcccaccaccactcccagctaattttttttattagtatttttagtagagacggggtttcaccatgttagccaggatggtctcgatctcctgacctcgtgatctgcttgtgtcggcctcccaaagtaatatattttttgtatagacgaggtctagttatgttgttcaggttggtcttgaacacctggcctcaagcagtcctcccccttggcatcccatagtgctgggattactggcaggagcccctgagcctgacttataccccttttactctcacctcttggttcctggatttgtgcattctctacagcagagatgacaccatatacttggctgcccaacctatgaacagaactccaacctctcagggtgttatctcttggcttgcaccctggctgaggcttcatcaggacatttcatcattctgtcaaactggttgctactagagggtgcaggtgatgtgatccatgaattatatgtattgagcccactgtctcacttcctttgctaaaaaatggttaggagatagttttcacaggaaaccttggctatatatcagttgcactataagtccatgaatgatggtgcttgcagaggcactgaaggcagggaagacatatattgtatatgtctatctttgtgagtccaagagcatccccttataatgggaggaatccagtggaatcattctgctaccacatacagccacctggtcctcttgccctgttggacagtgatagctttttgggggctcagcatcagccttggcttttgaagtttgggacttctttagtgctggaaagccagattccccctggtaggagaagagaatgctgaacctatatatatcctccctctgcaccactctgtgctttatatgtgggtccattaagcaaacatttggatggttgatgaaggatactgagtgacaatcagattttcttgttcactttattactgaagctatcctctgtgatggccttttatgagatttagtggaatagaattagattcacattctgtacttattaaaagagactcttccatatattcttcacctcctcctttgtgtccttcttgtctgtccttatctttctcctccttttagtcctcttcctgctcctcttactcatgcttgctcgtttccttctccttgttctccctgttttccttttcctccttctccacatgctccaccttgtcctcctcctaatcatccttctcttcatcctcttcctcatcctccttctcatcctccttatacttgtcttcctcctcctcctcctcatgctacttctccctttccttgtgctccctattcccccaggcctcctcatcctctacaactttctcctccttccttaaactgttctgcaataagcccatttgttttctccattttaatttctaagtactttttttgaaaaaaattttgttttttgccctccaatttatgttgggattcaacatagtctccctctatttttttaactttggtgcatgatattctttctcccccattctgataacactacacattgctttgacttccatccccattaataactaagtgaaaggtgttacaagtcttgctgaagattacacggtaattcttctcacagccaagcttgaactcagttttagattccagacttagtgtcttttcactaggctgaattgcctcatcatggctgaggatagaaatagcctcatatttagagtaaaactcagacaagaaagcaagcaagtgaacaacaataacaatagaaaacaaaaatttaaaaaggaaagagagagataggcatattcactaatttaactacaggaacttgaatttagtgttctctctgctcagtgatttaattctaggtgaattaaaaaagaagtttctgtgaaggtttgctgactttggtttgttccaaaactattaaaaatgtataactcttacctgctcaacagtatcatttgggaccaaagcatgaggcataataaatagagtcaataatatctgtacttagaggtatgagaatcattcctgatatattaatttaatttcttctgtaaaattaagggtgtttgtatacagactctgtagtgatatcaggtagtcaagacttggttttgtagtcttcttctgaagtcatcacaataaggaaattttaaacatgtaattagacaaattagattggaagatgaaaacaactgctttaattcaaaataatctcattaagtatatcacggcattaagttttacaaagcccaggtcacttcctctgtggctttccctatgaccacctttgtttctccttcagctgtaattgcttcccgctctggggcattttttatgcaaactacccgatccacctgtgctaaattatgattgtttattgtgttgtgcgtttaagatacagctataaaataaaaagcccacattttaagaaaagatagaattttttatgcggataagctttgtttcttaaaataaaaaagatttgatacttactcaatgttgcatcaagcgttcagaagagttttctgagaggtggggaagtctccttgcgaattaccttcagatcctattacagagaagaattcccatccccgatatgtgtgtagtagtaaagcactgttgccaaaaatgcccattcactctttttttgtatctctgggtaagtttaccaacctttttgtgctttaatttcctcaactataaaatgatggtaacagttgtactttcctcatagaattgttccaaatgaattcggtatttgaagtgcttagaacagtgcctagcacattataaatgctatgtgcctttgctgttactatttctactacgaccacattggtgatgacaatgatgatgaatcctattactaacagtttcccattcatttcatattaattaaaaaaatatttgctccaaatgcttttatctgtgcctacaaacaaacaaaaatccaaaggtgcagatttgccactaacgaaatagtaaagagatgtattgcaggatctaaggctgattctgaaagtgttggtccccaaagtgtttggagtggtgaagtgatattggagtaactgtaaaacctcttagatggttgctctgaagtaagcccctctctgttggatatatattgcatgttttttttttaatgtatccatatatattatgacttgtttctccaatgtaaatacacttcttgaagacagaagctgtgttttccatttctgtgtatttcctatagcattcaacagagtgttttacaaatttcagaagttcaataaatgtaatgattagctgcttaggtagaggtgagaggaggagaattaggaataaaatgttctccagttaattgcaaagtgcttttgtacttaaaaatgaatttgcattttatctttttctacaacttctggaaagtgttttagttggcatttgtcatttgattttgtacaattttgtaaattttctggaaatctaaattcctacaaagagtccatcacaggattttctattttttaattgtttctacatgaaaggaagtgttagtgtaaaggctagcctgttccatttacacttcatgcaatttgttaagaaaataattaagctttggagctggtagagaccatattgttacagcctaaaccctccacagggtacctggaacagaatggaaactcaataagttgttgataaataaatgaatcatttagtacaatgtcttcatttcacagttgaaggaactctgtgataacttgctccctgtcacatggtggcataggaggggttagaacaaatgttgctgatgtcattcatcatgctctagttgtggatttcacatttaaattgtaacataaaaaagactttaagtttagtctttgaataacttccttcattttaaatcttgtgtataaataaatttcttaggttaatatctgctacattttagaaaggcagtaggaagaagtttatacaagtcaaaagaattgttttaaagggaaagtaaaggaaaatatctgttagtggcttattcgaggtgtgtcagttgatgaatttcctttctctcaaccataagttttctttgctctgttatatattattagggtttcctagttactctaaaagactttctgttggaacaatttgtttttctcaagcacaacccatctccaacttgtctgtatttctgagactttcaagaaatgttggttatcattttaattgattttctgctaggatggaaaaaaagagccaagttcatgtacctgtttgaaggggcccaaactgtcagaaatagggacgattgcctggatgataacgctctgcgatgccctccacaatttcatcgatggcctggcgattggggcttcctgcaccttgtctctccttcagggactcagtacttccatagcaatcctatgtgaggagtttccccacgagttaggtaaggagctctgttcatattgtaaaagtctgcttacttcttgatttcagtggaatgagacttttcaggtgacatgctcttaaaatccttctgatgcactaaaatgaagaccttgttggacattgatgattatctttgatatcatccagaaataaaaaaaaaactattcacttgatattttcttcttcttcttctaggagactttgtgatcctactcaatgcagggatgagcactcgacaagccttgctattcaacttcctttctgcatgttcctgctatgttgggctagcttttggcattttggtgggcaacaatttcgctccaaatattatatttgcacttgctggaggcatgttcctctatatttctctggcagatatggtaagaagcttaaaatttgtattttcttttaatttaaaatttggaaatgagtatctggattataagttaactaagggtggccttctgctttccgcagtgtggaaggcctttctgttgtaattcagaacaatcccatttcaacagatcattctacttgagtagtttacactaatagtaatttgaaactgatcctcactggttctttaaaacataatcgtattgtttgaaggtgaaatgatttgtttttgctgaaatagagatattttcctctatctctattccccttgtggttggtacatcttgtcttatgttatagtatgtccctataggactttttctcctttttctccctcttttgtgtctatgccacatttggaagaggaataacttattagagtaaatagcattttgttatttcaaagttctaaacagtgacagctttttgggttaaactaaaactggtacactaaaattggtaaaattttgacaatagttatttgacatagtatatatttatatctgttcaatgtctgtgtctttgtatctgtgtgtatgtgtgtgtttgtgctcaaaagagcgtgcatggccgggtgcggtggctcatgcctgtaatcccagcactttgggaggccgaggcgggcggatcacaaggtcaagagatctagaccatcctggccaacatggtgaaaccctgtctctactgaaactacaaaaattagccgggcgtggtggcgggcgcctgtaatcccagctactcgggagtctgaggcaggagaattgcttgaacccgggaggcagaggttgctgtgagccgagattgtgccactgtactccagcctgacgacagaacgagactccatctaaaaaaaaaagagtgtgcatgtgtgtgtgtaagagagagaatatcctgtgcattttaaaataaatcatggaaatctttctataaaatgataatatggtagtgtatgaaaatcaaggactatttcttttgcatttggaactggatgtattacatacataatttaataatttgagttttttatttgcattcttacctccaaattatatttgtctagctctgttcgttgttctctgcagaccctcagaagagcaaggatttcatctcttagaaccaatgctacatttataacttttgtcacaaaattaatgtctcatgctatcttatagtcattttactttctgatataagaatggaatcttactagagttgttcagtttaaagaaataatactaagaaataatactcatcctggagtgagcaaaataggcacagttcaaggggcttttccattcatggggcttttcctctagaaagaggggatccttaaattaaacaacaaaaaatcacacagatgactaaataattacaaataataaaaaatgctatgaaataagagagaaagtagaatatatgatgagagatgataataaaggaatctagaataaactaggattagctagcaaaagtgttttagcaagatataaggtaaaacgtgaaggatttagtccactgaaaaattagagaaatacaagttctggtacaggtgatggtagatgtgaagattttgctgggtaaacttgaggtcatgaaagaatgggttgggtgaagcatagagagcaggtaatggaaaaggagaaagagaacataaaatgaggtaaattccatcccactgattgattcagttggagaaaaatactacttcagtacttttactcttgttgatgcctgaaagaactagatattcctattaagctatcagtactaaaacctttaaacatccaaagtaaatgatcataccacccctcccagcattctattacaggagcaccttgttttattgcacattgctttgatgcactttgtaaatgctaggttttttgcacattgaaagtttttggcaaccctgcctcaagcaaatctgtcagtgccattttttgaatagcatgggcttatttcctgtctctgtcacattttgttaatttttaaacgatttcaaaccttttcattattattataatatctgttatagtgatttgtgatcagtgatctatgatgttactattatgttttgggacgccatgaaccacattcgtataaaatggtgaacttaattgacaaatgttgtgtgtgttttctccctgcacccctgatcagccatccttccatctccctctccttgggcctccctattccttgacacccaacaatgttgaaattacaccaattaataatcctacagtggcctctaagtgttcaagtgaaaggaagagctgcacatctatcatattaaatcaaaggcctgaaatgattaggcttagtgaggagggcatgttgaaacccaaggcaggccaaaagctagactttgtttgccagatagcccagttgtgactgcaaagaaaggattgatgaaggaaattaaaagggctactccaatgaacacacaaataataagaaagcaaaacagccttattgctgacatgaataaaatttacgtggtatggatagaaagatcaaaccagctacaacattccgttaagcaacagcctatttcagagcaaggaataactctcttcaattctatgatggcttacagaagtaaagaagctgcagaagaaaaactggacattagcagaggtgtattcattaggtttaaggaaagaagccatctgtatgacataaaagtgtcaggtgaagcagcaagtgctgatgggaagctgcagaaagttatccagaagatctaactaagatcactgatgaaggtgactacactaaactaatagattttaagtgtagctgaaacagccttctgttagaagaacatgccatccaggactttccatactagagagaagtcaatgcctggtttcaaatcttcaaaggataggctgactgtcttactagggactaatgcagctggtaactttaagttgaggtcagtgatcatttaccattccccaaatcctagggcccttaggaattatgttgaatccactctgtgctctagaaatagaacaacacagcctggatgataacacatctgtttacagcatggtttagtgagtattttaaacccactgttaggacccactgctcagaaaaataaaagaaattcctttcaaacattactgctcattgacagtgcatcaaatcacctaagagctcttatggagatgtacaaggagatgaaagtttttgtgcttgctaacacaacatacattccgcagtccatggatcagtccatggagtagttttgacttacaaatcatatcatttaagaataatttgtaagtctctagttgctgtagatagtgattcctctgatgcatctgggcaaagtaagttgaaaactttctagaaaggacttatccttctagatgatatgaagaacatttgtgatttatggaaggagtcaaaatatccacattaattgaaattggaaagaagctgatttcaaccctcatggatgactttgaggtgttcaagacttcagggaaggaagtaactgcagaggtagtggaaatggcaagataactagaatgagaagtggagactgaagatgggactgaattgctgcaatctcgtgattaaactagaatgaatgaggggttgcttcttacggaggaacaaacagtgatttcttgagacagaatctattcctggtgaacattgctgaaatgacaacaaaggatttagaatattctataaacatagttcataaagcagggacagagtttgagaggattgcctccactttggaaataatttctactgtgggtaaaatgctgtcaaacagcattgcacactacagagaaatctttcatgaaagaaataaattgatgttgcaaatgtcattgttgtcttattttaagaaatttccacagccactcccatctttagcaaccactactctaatcagtcagcagccatcaagatggaatcaagactccaccagcaaaaagattacaactcacccaaagctcgaatgattggtagcattttttagcaataaagtatttttttaaattatggtatatacattgcctttttagatattatgctattgcacacttaatagtctacagtataatgcaaatatatctattatatgcactgggaaacctgggaattcatgtaacttgctttattgagatgtttgctttatcacactggtctggaaccaaacccacactgtctccaaggtatgcctctaagtgactgttttgtggtttgccacaagatggcaacagttatctaacaatatatctgtcttctagtttccagagatgaatgatatgctgagagaaaaggtaactggaagaaaaaccgatttcaccttcttcatgattcagaatgctggaatgttaactggattcacagccattctactcattaccttgtatgcaggagaaatcgaattggagtaatagaaaatggaagatggtgttgttaataaaggcatttaatagataaaaacatctccaaaaaggattttgaagctgatcctatttagttaaaaagataattttgctttcaactgtaggtccagaaaactaattattggcatcagtctgtgaaatagtccattatttgttgttaaaaatgcttcaaaaggttttcagtgtcagtctgagatgcctggtatataggagcctttgggaaatacctatttttcagtattccatgcatattagatatcaccatgaagcaagagacatgcattctataatcatgtagacactcagactcaggggaaaatacaagttatatcctgaaagcctttaaaactctatggtaggatcaaagattcaaatggtttcagagaggttttatttcaattaatttgttctagtgctttcaagagcaagtacatcaaaatgtagaaggtaaaatgtatgcaacactaatataaattattccaagtctttaaggagccaaagaaaaaaaagatttctcacagctttttgttctgttttgtatttcaattaggaacttgcagtattattttgaaaaccattctaaaataataggagttaggaaataaataaagttttgctagccctgctaagttcaggcttagaggcttatcgctaagtataaacttcaccagattccacgaaaagctggatagctttttttctgacttatgttgtggttgcacccctcacaaatggcagaacagtatgtaaagctggtaacacctcggtttcagtgcaccatgtgtttgctttgtgaaggtgaagaatatgttggtttagagaaagaaattggatgtaattttatgcaatttacttttaaagacaaacataactatttagcagagaatattttaataaatgcaaaacaacagctggactgctgtacatcaaggacagattaactggaaaacatatgttccttatgtgtgatcgagagccattcagaaaagacttcctttgtgttcagcctatacttttccatatggtataccttgaaaaaaattagcacaccatggttatttttctaccttttataaaagacagagcctgtttactcatttagaagatagagaaaattggtctaaaattgaacatcctagattcacactcccaagtcacttaaggtgatttgatggtgaggaaaatgattgacaaagcccaacaatgatctcaggaattacattttccaacagaccaaaaaatgttttcatgtagcagcaatgcagatttggtgaatatttaatatatattttagtatgtatttcactttatgactgacaattaaaaaatattgtttggccaaatagtaaacacccttttgaaaccatg

SEQ ID NO: 4 - DNA, 6834 bp, Homo sapiens
cggaaggggaagggggtggaggttgctgctatgagagagaaaaaaaaaacagccacaatagagattctgccttcaaaggttggcttgccacctgaagcagccactgcccagggggtgcaaagaagagacagcagcgcccagcttggaggtgctaactccagaggccagcatcagcaactgggcacagaaaggagccgcctgggcagggaccatggcacggccacatccctggtggctgtgcgttctggggaccctggtggggctctcagctactccagcccccaagagctgcccagagaggcactactgggctcagggaaagctgtgctgccagatgtgtgagccaggtaagagggggccttggtaagggccaggtgagtggcgaaagagagaggactggggttaatacagtaaataggcgcagggtgagactgagctcaagcaaggagggaaatcctgcagctgtggggaggcaccaccttgaagagggcagagaaccagcccttctcaggccttgatcccttaccctctcctcccaggaacattcctcgtgaaggactgtgaccagcatagaaaggctgctcagtgtgatccttgcataccgggggtctccttctctcctgaccaccacacccggccccactgtgagagctgtcggcactgtaactctggtgaggtgggcaagggtgtgtaggtggggacgatggacaagcatctgggggagcaaggctggtgacgggtttgggggtgcaaggaggatgacggggccaaagctttggccttcttcaaggctcacagcaagtggagccaatgctgggaaatgcggcaccctaggtggggcatgaattaacgtgggcagacatctagtattccaggaaagggataaatagaatttgagggattggtgagaacgggtctatggataggatcaagacaataaaatgagagaagtggctctatgaccccagatttcgggcaagcaacccccaaccaataaactgactgtgttcccagagtgcacatgtgcaggttggccattgggaatttggaatacatctttctaaggaaataacaaagcaacccaataggtaacaacctttttttgcacaaaagccaatttgagaaaatgagtatgtatgtagcctgtgtttttattttatagttccaaaaaaaaaggttttttttaaactaagaagtataatatctgcagttgtgcttatgaatgctgttattatttctccatcttcgctacaatttcagaaactacggccagtatacactagaatagcaggctttaaagtggcccctttgaacaaatgttctctagcaagacactttttagaaccaggtaactgtaactattaaactacctacttgcaaaacgtgtttggtgtcactgggtctcttggtactgggttttgttttgtttagtttagagacaaggtctagctctgtcacccaggctggagtgcagtggtgcaatcatagctccccacagccttgaactcctgggctcaagcaatcttctagtctcagcctctgaaagtgccaggattacaggtgtgagctgtagtgcctggtcccgtaaaaacaaaaacaaaaaccaacaccagatgtggtggctcatgcctataatcccagcacttgggaggccaaggcagaagggtatttgcatattttgtagagacttgagcccaggagcttgagaccagctgggcaacatagtgagaccttgtctctaaaaaaattggctggtcatggtagtgcacctgcagtcccagctacttgggaggctgagatgggaagatcacttgagcccaagaggtccccaagaggtccaggctgctcacatcactgcactccagcctgggtaacagagcaaaaccctttttcacacacacacacacacacacacaaaaagccagagcagtggctcacacttgtaatcccagcactttaggaggccaaggcaggcggatcccttgagcccaggagtttaagaacaccctgggcaacatggcaaaaccctgtctctactaaaaatacaaaaagttagccaggcgtggtggcacgaacctgtagtcccagctactcagaaggctgaagtgagaagatcacttgagtccaggaagtggaggctgcagtgagccgtgactgtaccactgcactccagcttgggagacagagtgagcagagtgagaccctgtttccaaaaaaaaaaagaaaacaaaagcttttccattgcaaatcaaccaaaaaggaacgtctttaatacacaagaaatattcttcagtctacttaacttaaaaatgtaactgtaatagatgcttttctcttgacagcggccccggcagggttggaacttttcctagtcaaaacccccctactcactctgcagacccttcctgaaggtcttcgccccatactgagcccatcttcagtcccttcacttgttccaaactagcccaatccatgacccttctctgggagctcagccaatagatcccaaagtaccacagcagcacctggctgcagaaggacccccgggaaatgcagtcaggggtgagaagatgggtgggctgaatgagctcggccctctctgggccttagtgctggcatctatgagatgaggacatgcctgaagtcagcctggaattttgagcccaggttatacctggtaaatagaactcccaggtctccggatactcaccactaaaacaatagcacctcattgtttttgttttgttttgttttgttttttaattatactttaagttctagggtacatgtacacaatgtgcaggtttgttacataggtatacatgtgccatgttggtgtgctgcacccattaactcatcatttacattaggtatatctcctaatgctatccctcctccctccccacaccccacgacaggccccagtgtgtgatgttccccttcctgtgtccaagtgttctcattgttcaattcccacctacgagtgagaacacattgtttttaaccctccccagcagcttctttaacccctccatttcccttttggcggagacccttgtcatcactcctctctaccaccagtgctctcccctcctgcactgtcccaggcttcctcattcttcccttgcaggtccttatccaacatggctcacaattcctgggaagccacaaggaaaccaaattctagtagcaactcagagtcagcattcattagcaggtcacagggccataaggatgccaagaaggaatttgtccctctccttgtcctgacaaaaggcataagtagaagaaaattgttaggtttggttatgacctttgacattctctctttgacatcctccctttctgcacccctcttccacctgtcagggcacctagggctccccgggtccccacaagtgtctgccctaaggcccaccaggctttcttgcagcctgtcccacatctgggtgtcactgccttcctctgaatcaccctcctctcaaaccagccgccagcgagcccagcccagcccagctagtgccctaccacacccgcccccaggataattaacacccagccacccagcatcggacctctacacttgagcaaaatgtatattttaaaggcaaattctcacgcctgtaatcccagcactttgggaggccaaggcgggcggatcacgaggtcaggagattgagaccatcctggctaacacggtgaaaccccgtctctactaaaaatacaaaaaattagccaggtgtggtggcacacacctgtaatcccagctactagggaggctgaggcaggagaatcacttgaactcaggaggcggaggttgcactgagccgagatcgtgccactgcactccaacctgggcgacaaagcaagactctgtcaaaaaacaaacaaacaaaataaaggctaagtaactgtaatcaagtaactatttattatttatttattttagtgtatccacaggttgtgcagccatcaacactatctaattctataacattttcgttaccccagagaaacttgtaccccttagcaatgactccccatttcttccccccaaccaaccccagccccagccccagcccctgggaatccctaatatactttgttttgttttttgttggtggtgtttgagacaggatctcactctgtcacccaggctggagcgcagtatcatgatctcagctcactgtaacctccacctcccgggctcaagcgatcctcccacctcagcctccccagtagcttgggcctacaggtgtgcaccaccacacccagctaatttttgtattttttgtagagacggggtttcaccatgttggccaggctggtctcaaactcctgagctcaagcaatccacccacctcaacctcccaaagtgctgggattacaggcgtaagccactgcacctggctattttctttctttcttttcttttttttttttttttgagacggagtttcactctgttgcccaggctggagtgcagtggcgtgatctcggctcactgcaacttccacctcccgggttcaagcaattccctgcctcacccggctattttatatttgttgatctgctgctggttatgcctgtgagccaaatttgtacaaattcttaagctttacaatcatgcgtgcctctctgtacgtatattatactttaattaaaagttattaaagtattggggccgagcacagtggctcacgcctgtaatcccaacactttgggaggctgaggctggtgcatcacctgaggtcaggagtttaataccagcctggccaatacagtaaaaccccgtctctactaaaaatacaaaaaattagccgggtgtggtggcacacacctgtaatcccagctacttgggaggctgaggcaggagaattgcttgaacccgggaggcagaggttgcaacgagccaaggtcacgccattgcactccagcctcggcaacaagagcgagactccatctcaaaaaaataaataaaaaataaaaaatgtatcagggagtacaagagaaagtcatataagctctttctacttttcatggtttctatgtctgaacccatgaatcttccttctgaaaggtccaactttcttctttaataacaataataatggcaaaggcattgggggaccgtgagcaaagggcaggcctttgcaggggtgggaatggaaagggaagcacgtccctagaggtgggcctgggatgggggttgggggatgaagcaagtggaccttgaaggtctccacaggtctgagtgtcctatgctccctgggcctctcttcccccaggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtggtaagttccaggcaactctctgtgccatcacgtggggtagcggtgataccccaaccagtactccccactcctacccctagataaggtcagcctgtttctgccttcccatcccatccagcacctctcaggccttcagatgtgccctatggggtcccctgctgctactcattctgtctctgtttttccagagatgctggaggccaggacagctgggcacatgcagactctggctgacttcaggcagctgcctgcccggactctctctacccactggccacgtgagttttctccttaatccccaccgctagagagaatgcatacacgaggggccaggagggaagccagacagaaagctcctaggattagggataagaggaggggaaaaagcagagtccactgtttaggagaggagttggccaacggtggcgggtgggatagaataaggtgggggaaaggggagaggcaaggtgacaggagggctgggctgagggagccaagggctagaccctcccctaacccctgtgtgtcccctcctattacagcccaaagatccctgtgcagctccgattttattcgcatccttgtgatcttctctggaatgttccttgttttcaccctggccggggccctgttcctccatcaacgaaggaaatatagatcaagtaagagacagaacacaggcctccggtcctgcccctgcaccacaccccactggctcaaccccactgcccacgcctggaatctcactgaaacccaccagctccacttcaccagccttggtcctaccccttctctccccttagctggcgtgctcctgacacccctcccccaagcgcacccgcctctacccatctccttctcccgtctccccctgcccccactgctggccaagactcatcggatctccttctgcagacaaaggagaaagtcctgtggagcctgcagagccttgtcgttacagctgccccagggaggaggagggcagcaccatccccatccaggaggattaccgaaaaccggagcctgcctgctccccctgagccagcacctgcgggagctgcactacagccctggcctccacccccaccccgccgaccatccaagggagagtgagacctggcagccacaactgcagtcccatcctcttgtcagggccctttcctgtgtacacgtgacagagtgccttttcgagactggcagggacgaggacaaatatggatgaggtggagagtgggaagcaggagcccagccagctgcgcctgcgctgcaggagggcgggggctctggttgtaaaacacacttcctgctgcgaaagacccacatgctacaagacgggcaaaataaagtgacagatgaccaccctgca

SEQ ID NO: 5 - DNA, 2667 bp, Homo sapiens
ctgggcccagcctatagtcagcggtgtctatgggcatggatctggacggggaaaaggacaaagcagcctccatccacagttcattccgggaccaggcccttgcaggcacgcgctgggctcctgtgggaagacactaagggccccaggacagacctcctctccgggcatctgggttcctagatggcagaggtggcagagtggggtgggatggcccaattgggagctttagcttccggcaaagagctgagcacagtacatcttcaatgtgtaagattctcctgggagaccagggcccagctggtggtgagctgggggaagtgggtgatactgccgtgggaggagccacctggccctctggggaagtgcactcgctgtctgcagcgcccaggcctgggtagctgggtgggggctggggggccatctgtgctcagggtgcctgcacctgggccttctctgccctgggccaagcctgcccgagcctctctgtcctctgcctgcccagctggacatctctgggcctctctggagaccagtggggtgggctgtgggggcgtcatattgccctggcttggcatccctcttgtggctgtacccctcccagcagccccaggactagcaagtccccgagatgggggtggggacagtggttgatgccaaaggttgtgggggcaggggcggggcaggagcaggaaggtcccctgagttccctcaccttgggcagagataaaaggagcacagttccaggcggggctgagctagggcgtagctgtgatttcaggggcacctctggcggctgccgtgatttgagaatctcgggtctcttggctgactgatcctgggagactgtggatgaataatgctggtgagtgtctggccctcggggaggcccaagctggggacagctaggcaacctgggaggggaggacgggggtggcgctgagtggggcatgtcggtgtccctgagcccatgggaggggaggacgggggtggcgctgagtggggcatgtgggtgtccctgagcccatgggaggggaggacggggttggcgctgagtggggcgtgtggatgtccctgagcccaagtgacagagcctcccctccctcgtgctgctgcagggcacggccccacccggaggctgcgaggcttgggggtcctggccggggtggctctgctcgctgccctctggctcctgtggctgctggggtcagcccctcggggtaccccggcaccccagcccacgatcaccatccttgtctggcactggcccttcactgaccagcccccagagctgcccagcgacacctgcacccgctacggcatcgcccgctgccacctgagtgccaaccgaagcctgctggccagcgccgacgccgtggtcttccaccaccgcgagctgcagacccggcggtcccacctgcccctggcccagcggccgcgagggcagccctgggtgtgggcctccatggagtctcctagccacacccacggcctcagccacctccgaggcatcttcaactgggtgctgagctaccggcgcgactcggacatctttgtgccctatggccgcctggagccccactgggggccctcgccaccgctgccagccaagagcagggtggccgcctgggtggtcagcaacttccaggagcggcagctgcgtgccaggctgtaccggcagctggcgcctcatctgcgggtggatgtctttggccgtgccaatggacggccactgtgcgccagctgcctggtgcccaccgtggcccagtaccgcttctacctgtcctttgagaactctcagcaccgcgactacattacggagaaattctggcgcaacgcactggtggctggcactgtgccagtggtgctggggcccccacgggccacctatgaggccttcgtgccggctgacgccttcgtgcatgtggatgactttggctcagcccgagagctggcggctttcctcactggcatgaatgagagccgataccaacgcttctttgcctggcgtgacaggctccgcgtgcgactgttcaccgactggcgggaacgtttctgtgccatctgtgaccgctacccacacctaccccgcagccaagtctatgaggaccttgagggttggtttcaggcctgagatccgctggccgggggaggtgggtgtgggtggaagggctgggtgtcgaaatcaaaccaccaggcatccggcccttaccggcaagcagcgggctaacgggaggctgggcacagaggtcaggaagcaggggtggggggtgcaggtgggcactggagcatgcagaggaggtgagagtgggagggaggtaacgggtgcctgctgcggcagacgggaggggaaaggctgccgaggaccctccccaccctgaacaaatcttgggtgggtgaaggcctggctggaagagggtgaaaggcagggcccttggggctggggggcaccccagcctgaagtttgtgggggccaaacctgggaccccgagcttcctcggtagcagaggccctgtggtccccgagacacaggcacgggtccctgccacgtccatagttctgaggtccctgtgtgtaggctggggcggggcccaggagaccacggggagcaaaccagcttgttctgggctcagggagggagggcggtggacaataaacatctgagcagtg

SEQ ID NO: 6 - DNA, 29223 bp, Homo sapiens
cgcgcaggcgcactgccgtcgggcgggcctagcgggcgcggtcattgtcctggcagagcggcgagccggtgagtgtggctgcggggtcgcgcccaccctctacctgtgccgggggccggaacctggccccggggagggcgcggcctcggggagacggcgtgcaggcccaggtacggggagtgcagggctgggtgctggtccagggcgtgcgggcgcaggagagggcgtgcaggccgggaccgaggtgcgcctggagaggctgagcggccgaggagaggctgaggggaagctgaggggctaaggagaggctgactggaggctgaggggctgaggagaggccggggccagggagggagaggcgtgagtgggttggaggtaggaaagttgcatgaacacgagggccatcctggggggaaggtggcctgtgtgagagacaggcagtgcatatcatcctgaggatgaggctgtgtctttgtggatattgtgaatgtgtctgtgtgtgtgtctgtgtgggtctgtatgtgtgtgtgagacagattgtgtcgtcctgagggtatggctgtgtctctggatattgtgattgtgtgtgtgtgtttgtgtgtgtgtgtgtgtgtgtgtgtgtgtgtgtgtatgagacagagagagaaactgcgtgtcgtccagaggctgggtctgtgaatgaatgtgaatgtgtccatgtatgtccgtgtgggtcatgtgtggtagcgtgtgtgtgtgtgtgtcccacttcttcagagtcttggtctctctgtggagctgtgtctctccaggtctctgcctatgcatgtgtggtgatatctctgagtgactgcctgtgtccttgtctttggtgttgtgggtgtgtcagccttgatctctgtccatgagtgtctctgtgggagggtgccctctgggtgtccactatgtctgctctttccatgcatgtctcttggcatgagtgtagggctgtgtctttccaggggccagtcctgtctggatctttcctatctctgtgtgtccacctgtgtctagagtatttcttgatgtttgcgtgtgactctgcaggggtgggtgcgtctgggtgtgcctcttccaaagactatatgcctggacaagggagcctagcagagccactaccttctcaccctacgcttctcctagtctgcctgcccacagttccctggctttttcctgcccatagaaggctctggaggcgtaggggcctgggagtgacaggggccgggtggaggaggggaaaaggaaggctgtttcttctgcctcccacaggatcttcttaaagtgggatcagactcccaaagggtctctgtgccctactctgttctgaccaaggagaggttgatttgttcagggaatgtgtatgcagccagtgctggccatttatagatgtcccagtaactctgaggtagagaccatcatcatccttatcttacagatggggaaactgagtctcagactaagtgactcacccaaggctacctcaggcatccaggttccagagcccccacaattaaccaccagacaaatgggagatcccttttttccctttccgctcagctgacacaaggttgcaggattttccccgagggaatccccgcttggaaacaatggaaatggaagaaaggaaggaaatgccaaaaagctcagaggaagaagaatccagatctaaggccaaactgggttcaacagccactggccatactcttgggcaagtcacttccagggccgctgcatgatttttaagggtactttgccttcatgggccccttgctccattgtttttttaaagcactaaaaattacattttgttatttcattaataggaagacaaatgtaatctaggctggattagatattttcttatgattttaaaagaaattaagaccttcctatgggcccctaaaggtttcatgggccccatgctctgtgcctactgggtaagtgggctctggtcactgctgtgatctggcaaaatgggccagtgttcttgacctcacaggacaggggtgaaagagaccactatctgcaaggaatttttttttatattaaattgtgtttcaaaaatgttgatggtaatatgaagtttctacacccagggaaacaattcaaaggaagagaaagaaaaaataattaataacttctacccccttctcattcccactccatgccctccagacaccaacccagcctcattccacagccttccccaggctggaagatgctgtacagatgtgtccacatccacatgttttggtttgttttttcacaaaggggctaaaggtatgcataaaactgggtaacttgcttttctcacttagtgaaaccacagacatgtcaacagatgcagaactaactctgcttcgcagaggcgacctgatacttcatactatggagaccctaccctcgatttgattcctttagtaatgtttattaattttgttttcagggtttttttttgcctctagaaacaatgctgccataaatatctttttttcctatatgtaacttttacacggagcttttattggtgtgggcagattcccaggaacagggttgctgggttatgggatttgtgtattgccagctcactttccagaaaaatgttgagcgactcacattccctcaccagtgtgtgagagggcctgttcctttcaatcctggccagcactagaagttagctcatctgttcattcagcttgatgtctaagtgcctcctgtgtgttagacgttgtcctagatgtcggaaatacaatctgaaagtgatcatccctgctccctgggggcttctgttctcatggagggaagacagatgacaaacttgaaaacaactattttaaaaaagacagttgtcaatagtggtttgtactgcaagaaaatagagggggagagagttgctgcttgaggtcaggtgattgggggtagaggacttctccaaggaggtgacgttggagctgaccaggaagcaggggtagtcatgggaagagcattccctccagagggaacagcagatgcagaggccaggaggcctggggccaagcatgggagaggggagtagaggagatggaaggggagaggccatcaggggcacatcataagggctatgtgggcagcagtgaagactttgagtgatttctaatggtgggggaagccaacgaagagttttgaagagttttagcccagaggacattttcactgagtgattacagttggccaggtgtggtgcccgggtacaggcattaagtggtaggaacccccagcccacacctttgtgatccttttgttctacaacagggcttctcaacctccacactactgacctttgggcctgatcattctttgttgtggggcctgtcgcgtgcatcgtacagtgtgtagcagcatccccagtctctaccaactaaacgcccatagcactcccctgccaaattatgacagtttggcaaatgtctctagacgttgacaaatgtctccgggggcagaattgttcccgattgagttccaaaggcttctgccaacttggccttatatatatgcccccctacaattaaaattacttttcacttgtggaagttggggccatttggcctaagaaaggctcttctttgtcttagtggttcttacgctttaagacgtgtcagaatcacctgggaaacttgcttagaaataccgattcaggggccctacccaggtagtttgattcagaggatctgaggtggggggcaggaatatatattctgagtgagattttaataagcatccccagtgattctggtactttgagaattggtgcaccagctacaagagcattgtgttggagaggattgaggcttttctgggtgatcttgaagggttggagatgtagagaggcatgtttctgtttatagcagaagaaaaactcatttccagtcagaggtgttggtggcacgggtagtgagcccccatcacgggagaagccaggactgagccagcatgtctcagactccaagaatttgtcctcatctgtgtcatggatcctatttttttactttgccatttacttgatttggctcactgattttttttttgtttttaatttaaatcaacttagtttaaaagggtattctgtgtgactgctgtaaaagaagctgagttttgctgtaagaagggaaacaaactttaaaacaaattgaagtaaagccaggtagttctaatgagatattatggtcaccctaactttggggcctgaaccctgcttttctttttaattttaatttttaatttttctgggtgtgtagtgggtttatatatttatggagtacatgagatattttgatacgggcatacaatgtgtaataatcacatcaggataaatggggtatctgtcacctcaaggatttatcctttgtgttacaaacaatctgattacatttttagtgttttgtttttttttttttttgagatggagtctcgctctgtcacccaggctggagtgcagtgttgccatctcagctcactgcaagctccgcctcctgggttcatgccattctccttcctcagcctcccgagtagctgggattacaggcacctgccaccacacccggctaattttttgtatttttggtagagacggggtttcaccgtggtcttgatctcctgacctcgtgatccgcccgcctcggcctcccaaagtgctgggattacaggcatgagccactgcgcccggccagttatttttaaatgtatcattaaattattattgatcatagtcaccctgttgtgctgtcaaatagtaggtcttattcattctttgtgtgtgtgtttttttgtttttttggggtttgttttttttttttttttttgagatggagtctcgctctgttgccaggctggagtgcagtggtgcaatcttggctcactgcaaccctctgtatttttttttttttttttttttttgagacggagtctcgctctgtcgcccaggtcggactgcggactgcagtggcgcaatctcggctcactgcaagctccgcttcccgggttcacgccattctcctgcctcagcctcccgagtagctgggactacaggcgcccgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcccggccctctgtatttttttaataccaattagccatccccacattccccccacccttccactacccttcccagcctcaggtttcttcatcattctagtctctatctccatgagtttgttttaatttttagctcacacaagtgagaacttgcaaagtttgtctttctatgcctggcttatttcacttaacataatgacctccagttttatccatgttgttgcaaatcacaggatcttattctcctgcttttcttttgtttcaaggggcgagtgatgggttgcagggatgttaagatatctctccacttgagactttatcctgagcttgtcaggagaactggaggagaattgaatcaagatgcatttctctaggcgaatctggctctgagagcccctgggtggaagttccttacactcatatctgctagttgaacctcatggtaaccccatgaagtctgggggaagtgcccaaagaggccacaggtggttctggggcatctcagggggcctcagtccagcactcacactcaaggagggatccgggcagggccattgggagaaggcagggtgtccggtggccctcagcagcctgcgggaatgtgtgtgagtgtatgtgtgttaaccatatctataggagtgtacatgggagtgcatctgtgaggcattgtagcctggaggctaagctgtggacccagacatactctgcctgactccagtcccagctctgtaatcttgagcaagtggtttaatctcctcgggcctcagcctcctcatctgtgaaatgggtaggtagaagtagtacctaccttccaggatggttggaagatcaagtgatatacattcaagttccctacatgcagtaagccctctaaatactcactgttgatgtatgtgtatgtgtgtctatctgtctgtctctgcttgtctctcagtgggtgtgtgtctgtcctgtctacatttgtgaggccagcctaggcctggtctctcagcatgcctccctctttcttctccctacagcactgttggtcaccgcaggcctgtcttcctattcaccgtgggcagcagagggatggctgctcagctgctgactgatgaggcactggtgagtcatgttgccctgtcactctcctttccaaaggctggatcctgccttgggtgagcctctgctgctcgaaccctctccaaggcccccagtcccatcttctggggctgtgtctgccctgatgaagagggctgtgcttatagatgatgctgaccctggctgtggggtcagttgtaggggacagggaagctttggaatggccaaactagctctctggactcttaggagacacaggtgggcacctgtgggaagtggtggtttagacatgtgacattgagtgaaaacttgcgcaggaccatgctggatgtgtgacctttaaaatgggcaaacacactaaatgcttcttgtgaatgtgaatgcccacatgtgtgatgaaagtacaaaagcttatgggaatgaggcccacaaattcacccatgattttaccccaaaagataatcactctggaatgtttgagtctggctaagcaattttaatgttaaagattgctttttcatcagagatttcatcctgagcatttctccacatcattaaaaactcctttgtaacgtccatcttaatgaccccataaaagtgttgttcagtggccataccctggtttacttaatggttcctctgttgttggacctttattggtttccaagtattctttttttggttataaacagtgtcatgagggacactgggcacaaatctttctttgtgtcttatatgatttctttaggagtgattccttgaagaggaagattgggttgggtatttttgaggtattttgatattatttccacatcagttacggcggtatgtattcccaccagcatgtctgagcgggtctctgatgatttatatggctgttgtcaggcatttattctgcatgttggatatatttagattggtgcaatattgcaccatgataataaatgataaatggatataaatgacaaatgctataaaccatgatgagcctgataggggacaaatggtgccacgtgatttatttctgagtagcattgtttgtattattagtgaagttcaagtcttagaaaatgtttactagccatttctgtgggcagggaaatgatccatcttgtgctttagcagtctacaggaatctgagtatgctttaaagactttacagaagcaaatgtgtgtgtgtaaatatttgtggcaaatattcttcccagatttgctttttgtttctttattttactttattttttctgtataaaagcatttcatatttttaaaatcaaatctgttggtttctcccactgtggtgtctttcttcatctttaagctgtgaaagggtttctccatccacaggtcacatagcagctatattttcttccttttcaccatttgtcatttgaatgttaacaatgaacttttgaatctacctgtaattgattttgctatataaatgtgtcagtagacttatttgggagctttggtttattctgtcttcatttgtttggtacctttgttgaggtacacatcacgtatcacaaagtgtacaattcactgattttgggtacattcatagagtcgcgttaaccatcaccacagtccatttcagaatattttcatcatcctgaaaaagagccgagtacccattggcactcatttccattttcccccatcagtccacggcccctggcaaccagtaatcagctttctgtctctatgaatttgactgttctggacatttcaaataaatggaatcatacatggtgtggtcttttgtgactggcttctttcactcagcataatatttgcaaggttcaaccatgtggtagcacatatagagcttcattcctttttttgactgaataatattccagtgtgtgtttctacatattttatttatcctttcatcagttgatggacatttgggttgtttccactttttggctattatgaataatgctgctatgaatattcatgtataagtctttgaacatatgttctcagttctcttaggtgtgtacctggaagtggaatgttgggtcatatggttaactctgttgaaccagtaaaggaaccaccagactgttttcctaagcgattgcaccatgatgccttcatttttgtctcatctgtgattcttgccttgaattcttcttttacctacattacttagaaagatttgttgattgtttttatttcaacagggttgggagttttggtttaagttttttttttttttaattactactagtgtaattacattcaatttttaaaaaaaattgagcctgtctggtttcagcgttttccctgtggtgaaatggtttgtgtcattagacaggaaaccgaaaaagatggagtactagaaaaaggggccttggacacgtacgtgcagagaactcagaggccctttgttagcacttctgtcccaacacgatgggctcccagacctggaaggctgtgttctgtgtctcagcacaaccctggtgccagtctgggcttggtgcctggagactgctcagtgaatagttgagtgaattaggtgggtgtacttagggaagacttccagaaggaattgttgtattacaattttgtaaaataaataaatataggtgtgagagggaacttttggaggtaatggatccgtttatggccttgatggtggtgatggttccacgggtgtatatgcatctgcaaactcatcaagttatgtatattattaaatatgcacagctttttatatatcagtcacacctcaatcaagtagtttaaatttttagaaaaggaggaattattagctgggctggctgaaagatacattgggccaaaaggtcaaataatatcatataaggatctacagttgtacataagatatatattaaatatcaacattaaataatagcaagagtcgacatttactgagagcttcctccatcacacgaggcactgtgtttggaccatatttgtacacatttaaccttacgaccctgtggagggggacattgttatcaggaagcctgcctctcatatgaggcttagaaagagaaagacagttgtccaaggaaacacagctcatgaggggcagagccaggattccgttgcagagtgtggttcctgagcctacactctgaaccaccagcccaactgctttctatcaaaggtgacccctcaggcagaggaggctatcttgagcccaagccccagaagggcaccctaggaccatagagggactagccagcgggggtgaggcctccccttgggattcagttggtggtggggacactcagtatgctggagagagccagtgagctctgagtgtcagtaacaatagctggcacttattgagtgcctgctgtatgctatgcattaccattctgagtgtcttacataaataagtgggcaggtggggcctgtagcaaattggagctttcttgccccatcaaagggtcagccaccactcagctccagcctagagtggccagatattctgctgtttaaagagaaactagaaattcattttcttggtaaattgtggatgtgtttttcatgtgagtaacaattaaaaacaattttgaaacccccataggagcaaaatagaaatgtgtacagctggcttcaggcccaggtacatctcatatgctctgtgtgctcagaactcccgttctttccttgtgccttgggggttgtaccactgaatcagttcagaacagcaggtccctctgtaggacacaataggtctctcattactcacatgccatgctgtgaggaagcccaagcagccacaaggaaaagtgatgtggagaaaaagagatgcccaccaaggcctcagctgtgccatccataccagctcaagagacagacttgtaaacaaagaagctagcagactactctagcccagctgccatccaactgccaccacacaagagatgccaggcacgaactgcccagctgagcccatcaaccccaaaatagtaataaattgttgtttttgagccattaagttctgagatggctgctacccagcaaaatataactggatcagaaatttaatctgtggccatggctcaagatcttcatgtggcagagtcatgcatggatgatgtgatcagccagcaataatgtgagacgtgcaggccttcatcacactagggaggaggtcgactgtcaagcaccatctctggaagtggcttcaggatcgcttgtgatctgtctggaagagaagcgccagctgggacactctgtgatatttatccaactgtcttctttataggcaaaagacgtgccatccatctcagggtctgatccaaactaaactgctccttggctgtgatgccagctagattggtattatacatggatgaggccatctaatgttggggctgagagttgaactgactaggtttaaaatgctgccactcccatctgtgtgacctcttgtgcctcagtctccccctttgtaaaagaaatgagctcataaaagtgcctacatagtagggttcttttgaagattaagtgagatggtgtgtgcagtgggcttagaacaatacctggcatgtagtatgtgcttaatgaacattagttatttttatcattatttactaatgaattagtatggaagaaacctggtgccaataccatgtctgtcttattcacagttgtatgcccagtgcctggtgcatggcaggcacttaataaatattcgtcaaatgaatacatgaaagggtgaagtatcactctgtgtgtttccaggaaccatcaatgaacatatttagaatatgttagaatatgtattagataccattgatctagaattcctgtttagggggtaaaaattcaaccatacttttctggtagcccggaaaattataaattaatcagttaaatttccgatccagttatattttgctgggtagtaaccatctcagaacttaatggctcaaaaactacaatttattactattttggggttgatgggctcagctgggcagttcgtgcctggcatctcttgtgtggtggctgttggatggcagctgggctagagtagtctgctggcttctttgctcacatgtctgtctcttgctgtggtatggatggcaccgctgaggccttggtgggcatcttttttttttccacatggattttccttgtggctgcttgggcttcctcacagcatggcatgtgagtaatgagacataatgagacacctgttgtgatgactggcttctcccagagcaagtgttccaggaggctgaggcaggagctggaagtcttctgtgatctagcctgagagtcccagaacattacctctgccagattctattggtcaagcaggtccctaaggccagcccacattcaaggatgggggaactagactccactagtgaaggatttgaggccattttcaacaagtctacccacttactattgtctaccaaagctgacttatattgaaagagacagtgaaaatgggtaatttgatccagcaaaatataactgtatcagaaatttaactggtgttttaatttacaattttctgggctaccagaaaagcatggttgaatttttaccccctaaacaggaattctagatcaatggtatctaatacgtattctgatatattctaaatatgttcattgatggttcttggaaaggtacctgcattttccttgcttgggcagcaggactgggcatttctgattcacagccctgagtatgataattgagagtatgagagttgtatgctcattttgggcaaatactcaaagcaggtagctttttatgtgtcttcctatcacatacgattgctttcttgggcctgttcaaggtaccatgtaagaaatttaaggattcagtcatttgttgtacaaacatttattgagtacctactaagtgccaggcgctgttctagggtctgaggattctgtggtggcccaggcagacacaacttcacaaccccccatgacgtagatgctgttatccccttttaacagaggaagaaactgaagcctgcagaggttaggtaattgaagatgttcactttcatacattcacatagctagaaaatggccaagccagggtttgataacaggtctttgtacttcccagacatgcccttaagacactgcactgtctttcaaaaaacaaacaaacaaacaaaaacactcacccccctgtattacttgtggctgtgtaacaagttacccccaaactcagttgcttaaagcaacaattatttactattttacacagtttctgagggtcaggaatggggcagtggcatggctgtggctcaagatcttcagtcttctcagggcttcccaggctgcagaatcccttctgagagcctcacacagtggttggtggtgggatgccttaactccttactgacattggtgggaggcttcagctgctggtcagatgggcctctccaaaggactctgcagggcagctggcttcctccaaagtgagtaatccaagggaaaaagagggagtgtgcccaggaccaaagctatgatctttataacctttttgtctttttttttccccctaaatttcttattgtgctaaaatacacataacataaaatttgccatcttaaccatttttaagtgtgcagttgagtggcattaaatgcattcctaatgtgcagcgccaccaccatccatctccagaactctttgatcttgtaaaactgaaactctgcagccattaaacaaacactttccatccccctcccctccgcccctggcagctgccattccacaagtctctataaatttgacttgatacttcatttaaatcatggaatcgtgcagtagttttacttctgtgtctggcttatttcgctgagcataatgtccccaaggttcacccttgtagcatgtgtcagaattttttttttttaaaggcggaataatatcccattgtatgcatagactgcattttgcttatccactcgtgcttacgtacacttgggttgcttccaccttttggcgattgtgaatagtgagtgtgcatataacctttccttggaagtgacaccacactaacctctgctgtatgttgttgatcacacagaatcaccctggcacagtgaaggggctacataaggctgtggataccaggaactggggatcattcggggctgtcttggaggctgccaacacacacacacacacatacacataaaacatgaaaatattcacagtgtcactcctctgatacagctgctgttcacattttgacacatttccttccatctttttcctgtgtattgttttcatgtggttgagataaaatggttatttagaattctggaaacccagaagcaactctacatggaaaagctctgcataatcttgtaatgaaaatagctatgtaattggccatgggggtaggctattatttaatgaatcccatgacatggatgttatggttgtccaaagacttgcccatataaataaaatggagtggacatctttgtgcatgaagctttgcccaatttttggattatcttgttgggatagatttccagaagtaaaatgtctgaacattttaaacacataaaatgtgtaatattaaccttttaattgaagaaacaataggtaaaacgcacctatttgaaaattagtaaactgggaaaatgaattctcccacttcccctccccatcctgaaactactggcatcacctgctcttaccagattctattttggttaagagctttattgaggtataattcacgtatcgtaaaatccatccatataaagtagacacattaatatccagtgctttttagtttatttacagttatgcaaccatcaccacaatattttagaaaatttttaccactccaaaaagaaaccccatacccattggcagtcactccccatttctctccaacccaccctcacccctaggcaaccacacatctaccttcattctattttgcctattccggacattgtatataaatgaggcatacgatatggattttgcctgtcccggacattgtgtataaatgaggcatacgatacagattttgcctgttccggacattgtccataaatgaggcatacgatatggattttgcctgttctggacattgtacataaatgagccatacgatatggattttgcctgttctggacattgtacataaatgaggcatacgatatggattttgcctgtcccggacgttgcatataaatgaggcatacgatatggattttgcctgttccggacattgtacataaatgaggcatacggtatggattttgcctgttctggacattgtacataaatgaggcatacgatatggattttgcctattccggacattgtgtacaaatgaggcatacgatatggtcttctgtgtccggcatctttcacttggtgtcatgttttccaggttccttcatgttgcagcgtgtgtcagagctgtcgtgtggacgaaccacattgtttatccattcgtcaggagatggacatttgagtcgtttccactttgggactattgcgagtaacattgctatgagcatcagtgtatagtttttgtgtggcactctgttttcatttttcttagatatctaccaagaatggaagtgctaggtcataaggtaactctgtgtttattcacttgaggaaatgccagactgtcttccaaagcagctgaaccattttacatccccaccagctgtgcattcttaccagtttcttgcattcacttccataaagaggtctgcgcctgtataagcacatatgtgtaaatttccctttattatttcctcttatggaaatgttaacaaactagacacactgttctgggcattgatctgttttttcatgtaacagtatattcgggagatcattctgcacctgcagctaaggggctgccccctttttttttgttatggctgtgtgtattccactgtggtggattcactgacctgatttatccaaacccctactgatggccatttgggatgtgttcagtctttttacttcttcatctgatactgtaataaatatccttacacatctatgtttttgcatgtgtattaatatatctgaattagtctaaagtgttcaaaagaatttgaatattagttttgatagattttgctaaatgccactccatagacattgtcccagtctgttctcctatcaggagtgtcagagaggaatgtttcagtatttgagttggagagagtatttcctttagagaggaatgttgttgcagtatttgtccttttgtgactggcatatttcatggagcagaatgtcctcaaggttcacccatgttgtagcatgtgtcagaatttctttccttattaagtctgaatgagatttcattgtatatagagaccacattttcttcttctttttgtctgttagtgtacacttgttgcttccaccttttgtctattgtgaatagtgctgttaggaacatgggtgtgcaaatacagagtgcacctgtaaaattctaaattagattctgtccctctcctgctcagaccttccaaagaatgaataagaaggagccagcttaggccagacctgaaggaagagcattttgggcagaagaaacggctagtgcaagttccctgggggatattcgaggagagaaggcaggctggcagggctggagcagagcacaaggggcagagcagttgtggggacagtgtgggggtcagaggtcagagtcaggactacatgtatgggcaccacaagttttcattacctgccgctctgggtggttcgtgagaggcagcagcctgcaaggatgaggactcgggctctggagacagcccatgaggttcaaagcctggctgtcccacgcagtagctgtgtgctttggtccaggaacctaccctgtctgtgccttggtttcttttatctctaccttccagcctccctccagtgccccctattgacagagcttcacagggagtagctgctaaggcagaaacatcacgggatcgcagagtcaaagcccgcagatctctgtgcagagtgtggaaggggggcttttggggatgaaagacaatcacttaatcacttttaaacaggcatactatattataatgttagcacctgactcgtaggtctgttttaagttaataatgactgaatcaatatgtgccagcacggtatctatttctcaaggctgctgtaaataagtaccacagactgtgcagcttaaacaaaggacatttattctgtcacagttctggaggctggaagtccctgatcaaagtatccacaggaccacgctccctctgaaggctcaagggaaggctctgttccaggcctgtctcccacctgctggcaagttccctgacttgtggcagcataactccattcttcacacagccttgtccctgtgtacatgtctctctctgtccatgtttttcctttctgaaatgcaaattaaaaccacaacaccaccttaccccagccaggatggccattattaaaaagtcaaaaaacaatagattggcgtggatgtggtgaaaagggaatgcttatacactgctgctgggaatataaattagtacaacttctttggaaaacagtattgaggtttctcaaagaactaaaagtagatctcccatttgatccagcaatctcactacttggtgtcaacgcaaaggaaaataagtatttatatcaaaaagacacctgcacgtgtatgtttattgcagcacgattcacaattgcaaatatatgcagtcaacctaagtgcccatcaaccaatgaataaggaaagtgcccatcaacccatagataaagaaaatgtgatacacacacatataatatatatgtatatcacatttatacacatacacacatacatacacacacataccatggaatactactcagccataaaaaataatgaaataatgtcttttacagcaacttagatggaactagaggccattatcataagttaagtaactcaggaatggaaaaccaaatgctgtgtgttctcacttatgagtgggagctaagcgatggatatacagaggcatactgagtgttataatagactttggaaactcagaagcgggcaaggtggcaggggggtgaggggtgaaaaactacatattgggtacaatgtacactattcaggtgatgggtgcactaaaatcccagactttaccactgtacatttcatccctgtaaccaaaaattacctgtacccctaaagctattgagattttaaaaattgaaaaacaaatttcccttttctataaggacaccagtcctgttggattagggccccctacccccactccaggatgacctcattttaactgatttcatctgcagcagccctgtttccaaataaggtcatgttcttgagaccttgaaggttgggacatcagcatatgaattggaggggctcataattcaacccataccaaccacttagagtagtacctggcacatagcagctgctgcataatgactggtgttgcctacctcatcatcaccatccacactgctccgccaggttctttcagtccacaggcttgtgacatctaccacaaagtgctgctccttccacgtggacttctctccactccccagcagtctcatctgccttttcaccttcgggtcttgatccaagctctcttccctacccagaatctcccactaaagacccaaccaccagttctcttcacagactactcaaagtcttcctgggcaccccccacagctggcattactcaggcacctggagacctttgttcataccttcatgattgattgcgtttaccaaattacgtgtctttgtctgcctctgccatgaggaactagttcttgaacattttgtgtctgtatcccagcactgggcttgctgcctggtgcttagtaggggctcagaaaagttagctgtgcccctggattccatctggggctctgcctctgctgtgctcctaatgcatttgttgattcactgggttagactcactttgccttcagttcctttccatctggttctcagatctgtgaccttctgttccagaatgtttcatatgggatttatttctctatcagagtgccaacccaatataactagacacagagaaaactgtgagtgatccctttagaactctgcatcttattgaaatcacttcagctacatcccatctgcctcccctaaatcctcttggcttttctggccattcctccacgcctctgtaaggagggccctgcgatgagcggatgtgggtttcctcttacaggaatcagtgacgttcagggatgtgactgtggacttcacccaggaggagtggcagcagctggagcctgcccagaaggacctgtacagggatgtcatgctggagaactacaggaacctggtctcactgggtaaggggcagcttcgttgagttactcatcattggcccataacttcaggaccacaaataaattaaaaaaaaaaaaagatcaacagatctgactacatggaaagttttaaaatttgctctacttgaagctaaaaataaagttgatcacgatggtatgggaaataatatttctcacgtgttttacattcaaagggtcataattatgtaagcagctcctgggaataagaaaaagactaagcgaataatagaatggaccaaacttatgagctggtgattcatagatttagaaatacacaaagttaacactttataaaagttttttaaaccttactactggagacacacaaattgaggtactagcagtttttgcccatgaagtgcacaaagatgaaaaaagaataatagcccatattatgaggctgtagaggaaaggactctcattttaggagtataaagttgggacagctcttttggaggatagcttggccttatcagtcaaatattgaaggattccatttttagagatctgtcctttagaaattctcatgtaagtatgcaaagttttccatgggaagaaaaatcaaggaaaacgacctaaatgcccagctgtctaagactgactgaacgtatcaaggcctatcgatatgttggaaaccaggcagctcttcaaagctgaggactgtttgtaggcactgccatgaaaagatgtcccacaacatactgttaggttaaaacactcacttttgtaatcagagcgtggtcctatttctttaacagcttttctggaggtattattgacctgtaattaaccacgcatatataaagtgaagagtttgatgcattttgacatagtgcatacccaggaaaccatcaccatgatcaagataaaggacatatctatcatccccaagagattcctcatcctcctttattacccctctctccccttcctcaacccccatccccagggatcccctggtctgctttatgtcactgtggttgagtttacctgctctccagttttatatacatggaatcatacagtatgtacccttttttttttggtctggctttttttggtttgacttggcattattttgagattcacccatgtcattgcatgtctcatagttcatttatttttatctctgtgtattattccactgtatgtgtatgccacaatttgttcagccattcgcctggtgatcaacatttgggtcatttccagttctgggctattacaaataaagctgctgtgaacatttgtgaacagttctttgtgtagacatttgcttttccttctgttgagtggaaagtctggatcatgtgctacatgtatatttaacttgtttaagaaactgccaacctgttttccaaagtgattgtactatttcacatttccaccagcagcgcacaagcttcattctaattgctttatcttttaaatttagcctcccttgtaggtgtgtggtctcatcactgtgcttttcatttgatactgacacttgtaggtgtgtggtctcatcactgcgcttttcatttgatactgataatcttttcctgcgcttatttgatgtttgtatattctctttggtaaagtatctgttcaagtcttttcctgtttttttttttttttctgttgggttattattgaattttgaaagttcactatgtattctttaactatctagaagtccaatgcgctatccattgcgtcacagagccagaccactatgtattcttgatacaagattttctttagatatatgccgtgcaaatattacctcccattctgtggattgtgttttcactttcttgatggcattatttctgactctaaagtttttaattttgatgtagtgcgatgtatctattttttgttgttctcgttgcctgtatttttggtggtatatctaaacaatctaactcaaagccatgaagatttattcctgtgttttattctaaggttttagtggtttagctcttcactgaggtctttgatccattttgaattaatttttgtgagtgatgtaagaacggggctcaacatcattcctttgcatgtgaatatccaggtgccccagtgcaatttgttgaagagcagaagtttttaattcaatgaagtccagtttatcaattttttttggttttggtgtcatttgtaagaaatcttggcctatcccaaggcctcagaggttttcttctgtaagttatatagttttaggttttacattttgttctttgatccatttagagttgaattcttaatatgatgtaaagtattaatccaagtttattggggtatgttggggggacaaatgtatgtccagttgtccaacaccatttgttgaaaagaccactttctccaatcgcctttgcatttttatcaaaaatcacttgtccatacaagtgtgggtccaattctgaatcctgttctgtccatctgtttgtgtgcctttgcaccaataccaagcaatgtcatcctttcagatcttgcttttatgacttgtgcccagtctagaatgacattctcctcaagatcattctgggttttctgcccaaagccccatgaatgacaaggttttacagtctgattggtaggaaaaggcactgttcccagccctgtgtcagctccggtcactgttttcaaatcctttcagatgtttttttcctgtgttgggtagtttcctgacatgcacgttttgaccaatagtctgctaaatactggagggagttccttgcaggtcttcagaacactgtgcagctcattcctctctcatctgtcctgggaactctatctgccttggtctctcagctctgtctcctcaacttggggagttggccagactgtacctcaatttacctttcctttgtcagagcctggaaacttcccaggcagttagctggggcagttgtaggacccaccttgtttgattttcttctctcagggattttgcccttcattgcctgatattcaatgtcttcacaaccattgttttatatattttgttttaggctcaagggaatcttatcctttatactctgtcttggacaaaagccccatttgggggaaaaatgtgtgtgtgcatgtacaggcgttggaggggagtctaaaatataaaccaaactgttaactctggtgagtaagattacaggagacttccattttcttttgtttttttttgttttttttgagacagtcttgctctgttgcccaagctggagtgcagtggcatgatcagagctcactgcaacctccgcctccctggttaaagcgattctcctgcctcagcctcctaagtagctaggattacaggtgcctgccaccacacccagctaattcttgtatttttagtagacatagggtttcaccatgttggccaggctggtctcaaactcctgaccaagtgatctgcctgattcagcctcccaatatgctgagattacagagacttccattttctccattactgttttggtgtttgatgttttaaaaacaagtgtgaattactttctaatcagaaacaagcatagaaagacagaaaactgaaaaagtgtgaaggacgagatttagcaatgtgaaaaaagaatgcacctacatttgtatcaaaaggaatgaatagcgttttgcacattaacggcttttggcgaaagtgccctctgcagaagcttcggatgtctcccatgacagtggccatgccgtcagacttcctgctcttttgctgacctgactagagaaaaggtgtgatcgggtagttttaatctgggtggtgtggaactgtgttttcacagctgtcgtggttaagggctgagtgtttgctgtttgcagatgatctctactgcagggttttagcctgagaaagggaggtggcttcctcccctctgtccctctgctggtatgctgcccccagctctcctgatttctccaggtcatgttcactccaggagacttccagctgtattcacaaccaagactcctggtcctctccgatagctgccagctcatattccagagcatctcagactccatcagccccagacaaaattcaccatcacccctggagacagtcttctctccaccgagtctacaccccacactaccatctgtgccagcatctgggcattgtgccacaccctgactagggtctctaaaccacaatcattctcctgcatgaagtatttctcagttcttttcctctgagatgggaaaagtgatgcatggtaatatagccactcctcaggagccacagctcgggggccacactgccagggcttgaatcctgctgtgcctcaggtgtattgtgccctcgagtacatctctgactccattcttgtatctccgtttcctcatcagcaaaatgaggatggtcataactatctactactgtatgtaactcaagtatgattgtgagggtctccagagatccgtgcaaattgttcagcactgtgccaggcgtgttgccagtgctccatactcgcttatctggtaccttctgtgtgcaggctgtgtgaggagctggggggtgagcaaggcccttgcctctagtggagctgactcctgctgggggccgataataatcagatcaagggacaggcagagaaaaattgccacaggtcactgagtgttcataaagaagtgggcaggaggaccttccttccttacgtagggtggattgggaaggctcccgtggggttgtggggaaacaggcctgaaggataaggacgtccacggcaggtccacaggacagtggatgcagaggccctgaggcaggaagaacacacggacaagagggagagcatggtgatgagaaactgggagggagggtgggcaggggacgactcaggcaggcctgctgggtgctgagggccctgaggctgctggaggtcctggcatcattctcagtaccgtagggaggtcacgggagggttgatctgactcctcagaagaccgcactcccggaggagtggaggacgcattgcaggggtgagaatggggtggggaggacagagaggagctgctgtcgcatttcaggcaaggacccgtggtggccagccggctttttgtcagtggggaccgggagatttggtctgattcaggctgtttgcgcacagcaatgcttgccagcagtcaggacggtgggagggaaagggcaagtcaggatgtctgctaagtctctctaaaacaattttttaaattttatatttaaaactgtgaaaaatatatagagaacataaaatgtaccattttaaccacttataggagtacagttcagtggccttaaggacattgatgttgttgtgcagccatcaccgccatccatccccacagctcttttcatctcccgcaaatgaaaccctatccccattaaacaccaactcccacccactcctcagcccccggcatcctctgttctaccgtctgtctctgggaatccgacctccaggtaccgcagatgagtggaatcacatggcatccatcctttcatgtctgatttattttactaagtctctttgatgggctttgcttttttaaaaaatgtttttcatggggaaagtctgaaatcccaagtttcgtatcggatgtgttaggcttgggctatccagttgagatgctgggcttttttgaacagtttggacatagcccagaaagcaggtctgggctgaaggtgtatgtttgagcagcatcagcctgtggataacgtttagatccagactcactttgtgtggctttgaaggtccagtgtgattcaggagagtggtctcaaactcaggtggcctcaggaactaccaggtggatcatttatgtggaggagggggacagctgctgccccatgcaatgtcttgttgcccataagaatgaggctccacgtggccatatcttcatgttttttatgagctagcagccacatggacagcaacttttaaatgctgggaatgagtcccctgatgcagagtctagactccacctaggctagaaatcgaggtttagaaatgaacaggatacacgtggaaggtgattgaagctgtgggcacagaggtatttagccagggagggtggatgagcagagaaagagggtctgaacccaagctccacggaatgcctgccacccaagaactaggcagcgtgtgacgtagcaacagaagcaattgtgagaccccaagtcatgtgattattgtgggctcaagtgagcggtcctgtgtgtatgaacacttagtgctgaggctgatgagagggctggggtgcgggagggctggggccagccagggcaggtgcgtgatgagctagcgggagccggagccccagccaggggagaggatcttgcagccagcaaccctggggcagccataccagagaagtggcaaaggggcaggagaaatgcccgatttttctctcatttcacttccagtttcctgcctttggccaaacacacaggaagccagtgggcaaagtcagcatcctgagcacagggcaggtcagagaagggtggagaatagaaagtgggtcgaaggagtagagaataaccaacacatgccatataaatgctagctatttattatttctttattctttttcttcttttttcaatatttatttatttatttattttttaagagacagggtctcaccctgtcacccatgctggagtgcagtgacgtgatcatagctcactgcaacctcgaattcctgggctcaagtgatcctcccacctcagcctctcaagtagctgggacaataggcatgtgccaccatgcctggctaatttttaaaaaaaaatttagtagaggcgggggtggggggtctcactatgttgcccaggctggtctcaagcaatccttgcctcagcctcccaaagtgttgggattacaggcatgagccactgtgcctggccatcttattatttctgataaaaactgttgcttgaggagcacgactggttgtggatctgcaggatgaagtgaggaggccctgcaagtcagtagccggctgcagtcattcctactcaggtttgcagagcctgccctttgctccctgcaagcccagctcactactgcagtttctctccaggagtgcctgtcatccctcttctgaagggacatttgctctccctgttcacctcctgtgttgactctgcttcataaaccacattggcctcagttccatgcctccctcctccctccctctctgatccaaggttcttctttgctaactcgctctgtgccttgggcctcattggggtcggtttcatttgctccttcctgctgtggagcttcctcactcccagcatctttaactacatgtatttgcatcttctgtttttgtttttttcagactgggagactagacctgaaatgaaagagttggatccaaagaatgacatttcggaagacaagctctccgttgttggggaggccacggggggacccacgaggaatggtgccaggggtcctggctcagaaggagtgtgggaaccaggcagctggccagagaggccgcggggagatgcaggtgcagagtgggagccattgggaattccccaggggaacaaactcttagggggctcagtacccgcatgtcatgaactgaaggcatttgccaaccaaggctgtgtcctggtcccaccacggctggacgaccccacagaaaagggggcctgtccacccgtaaggcgtggcaagaacttctccagcacttcagacctcagtaagccccccatgccctgcgaggagaagaaaacctacgactgcagcgagtgtggcaaggcctttagccgaagctcgtccctgataaagcaccaaaggatccacacgggagaaaagccgtttgagtgtgacacctgtgggaagcacttcatcgagcgctcgtccctcaccatccaccagcgggtgcacacgggcgagaagccctatgcctgcggggactgcggcaaggccttcagccagcgcatgaacctcactgtgcaccagcgcacgcacacgggcgagaagccgtatgtgtgcgacgtgtgtggcaaggccttccggaagacttcctctctcacccagcacgagcggatccacacgggggagaagccctacgcgtgcggggactgcggcaaggccttcagccagaacatgcacctcatcgtgcaccagcgcacgcacaccggggagaagccgtacgtgtgccccgagtgcgggcgagccttcagccagaacatgcacctgaccgagcaccagcgcacgcacaccggggagaagccgtacgcctgcaaggagtgcggcaaggccttcaacaagagctcctcgctcaccctgcaccagaggaaccacaccggcgagaagccctacgtgtgcggcgagtgcggcaaggccttcagccagagctcctacctcatccagcaccagcgcttccacatcggcgtgaagccgttcgagtgcagcgagtgcggcaaggccttcagcaagaactcctcgctcacgcagcaccagcgcatccacaccggcgagaagccctacgagtgctacatctgcaagaagcacttcacggggcgctcgtccctcatcgtgcaccagatcgtgcacaccggggagaagccctacgtgtgcggcgagtgcggcaaggccttcagccagagcgcctacctcatcgagcaccagcggatccacaccggcgagaagccgtacaggtgcggccagtgcgggaagtccttcatcaagaactcctccctcactgtgcaccagcggatccacacgggcgagaagccctaccgatgcggcgagtgcgggaagaccttcagccgcaacacgaacctgacgcgccacctgcggattcacacctgagcgcctctgtgcagggctctcactggcggtgcccaggacggacgccagatggctgcgcgctttgtcagcagtgctgtgagaagttctcccctggggtggggactcggggtcaggggagctcaggaatgtggggttgtggaggggctggctgatcacacatgccccctccttactgagcctcagaaagcaagcccctcggtgtctgacgtatctggggacacttcaaggttcactgtagctgagccatggccgctggtaacagacatcagggttccagactagccctcttgagtaactgtcctagagctgggacaggtcacgcctgcctccacatctctgtttcttcagcgggaaagtggagctggtggaggggagggaggagacatctccctctttgtcagatacatcctggagtccagatgcagcttgtcaccatctgcattttctccgtgggttgggagcgtgagggccttcctatccctccacccaccgtttccctgcagtgcacgctggcagccaccaagctaggaagtggcaggagtttgccggttgtttgtgaggggaaaaatttttttttcagagttagcaaataacagtaactactgctttgaatgccaaagtttcttagagtggtctgttacgttttttaaaaataaaacttttaattaagggagactatgttattttaaacagtcttctgttttggccagttgtctggtcaaaatcggcttcagtgattgtgggaggggcaagaaggaaaagaaacagcagcaggtttgatattcagaaagaaatatgcatggattcaataagtatatacatctgagcacagattatttttatagtgatttttgcagctaaatgtgaaggcaactgtcttaagtcccttcctgctgctgtaacaaaagaccttagactgggtaatttataaacaatagaaatttgtcactcatggttctggatgctgggaagtccaagatcaaggctctggcagatttggtgtctgatggggtatgttcttcataggtagtgcttgtgtcttgatgtgtgtcttcacgtggttgtaaacggcagacttcctcaggccccttataagggtgctaatcccagtcacaagggccctgacctcatgacctcccaaaggtcctccctcttcatactgttacattggggatcaagtttccaatatgtgaatacagggaagcaccaacagaccatatcagtaacttcgagtgagtttcctaaggccgactgaagctctgtttacacataagagtgttccccaaagtgcactttctctggggccttttaggccatacttttaattttgttatttgatactactggttgctaatgttctggcatttccagttgttccacactccctgacactggtgtacacaaataaataatacagaaaaaaaattatatcagcaagcacattctgcaaataagaaaaacagccactcgggcacgtataaacaaggtggaccgtttttcaaattctggcagtcaagtgggcctcagggtgggtttgattcaggctctcatcattggagccatagctgtggttctctgtgatgctctggctctcccttcatacttcttaacatcatcctcaggctggcttcccacgtgggattgtggtggtttccagcagcgagtcttgatcagacattgctttagttgcatctgttgtgggaataaatgagaatatgtatgca

SEQ ID NO: 7 - DNA, 65487 bp, Homo sapiens
aggcagaagccggcggcgcgcggacagccagtcggcgccgcgcggagctggccgctggattggctgcaacactcgcgtgtcaggcggttgctaggctccggccgcgcgccccgcccttgcgctcagcgccctctcaccgcccggtacgtgctcgcgcgaaggctgcggcgcggcgctcgcgcctcttaggcttggcggtggcggcggcggcagcttcgcgccgaatccccggggagcggcggtggcggcgtcctggggccaggaggagcgaacacctgccgcggtcctcccgccggcgctgggtgagtgcaccgcccgccctatccgctgtgcctggctgcttgggcccccatccctgctgccggctgagccttccgcggaacacctgctgctgctcgctgttcggcccggggcggggacacggccgcggacgtccttgtgggtgaggaggcggtgccgcccgggcggagccgcccctttgctctgggtcccgcaggcaaaggctcggagcgttcggctgcaaggcccgggccgcgcgccgcggcgatgggccaggccttagctcggcccgcgctcccgggaacgtggagcctgcgccttggccgcggtcgtgagccgcctgcgggccgcctccctggcttccaataggggttgtggcccagttgggccggattccgactgtggggagtcgagccccgctagaggcagggaagggcaccgctgccgccactgctctgtcagagctgctcagagccttggtgggttccgtcatccttacttgagctcctttggaagcttgtttacttactaccttttcactttgtatttggcagacctggttcacctgcgagtgtatttggatagtgtctccacagaaaatgtcgtctcggcttggaatctttttgccataaattctaagttagtttcctacaaactcaactggtaacttaaaatgcatcctttaatctctataacttgaaaagttcttatttttgtaaatcgagttgcttcagaattagttttaatggtgacgatgaagttttgactggttgaggcaagagcctgggacagcaccttcatagcgcagagtctaacttaagattctagcctgctgcttccctaaataagcgagtagaagtgactcctggcatagaagccagactccgtgaagggaaggtggtgtttataggacataattccagataaagagctggatgtgctttcaggtgttttgcaaagtctggtgtagatggggtattgcagttttatcaggttctatgcttagttaagttcagagacattattttggaatgttcctgttctaaaaaagaattcaaccgtactacattttggaggcataaaaatccatagtttgagtatggagaagtttagttgaacttttgtgggttttttttgctgtgaactaaggaagaagagctcatggcctgtgtcctgtattttggagaaatcagatttcagtagggagttggatccatcatgtgaagaccctatgggatataaaagcttgtgctgacatgtgcttttgttaaaagaggcacagtgcaaatcctcactgttcttaaaaattgattttgcaaaatgcatttattcatttgaacaaatattttgattcactacctgccaaattctgtggtagatcctagggacccatgtaaaagagtctgcttctgcccttaaggaattcagtttagggggcagtggggaccagggaacccatgattaatagtgtctgcccgctaagcgtgctcacatagtagcctgaggagagagggaattcctgagctgtggttttcagggccagcaggaattagtgggggacatcaaggcctggacacagggaaccacatgtacatgagcacctaaaatagtgcttgggagtcctccagcttggcaggtgcgagctctgggcctgccttgtgctcaggagttccaactttgtatgcggggtgcaggagtctggggagccttttaatcagtagagtggtgtgacccagacctgtgtttagaaagatattgctggtggagtggcaggaagagggagggggtcaggacctctggcagttaagtcagctaacagaggagaggtggggagggagaaggagccatcagaaaagagagactgaaaaggtagtacagaggggatgtcctgccatgatgggggaggagctacattctggaatgcatgggccagaagaccaagcataaccaaggatttaaattccttgttgactacctggtatagtgacctacagagttgcttgtgtctggttagtgttgaggatggtgggatttggagggaccacttagggaccacttagactgccgtcgatgcataggaaaggaggcctggcatttgattattaaaaactttatcgccgggcatggtggctcccacctgtaatcccagcagtttgggaggccgaggcgggcggatcacgaggtcaggagatcgagatcatcctggctaacacggtgaaaccccctctctactaaaaatacaaaaaattagccgggcgtggtggtgggcacctgtagtcccagctactcaggaggctgaggcaggagaatggcgtgaacccgggaggcggagcttgcagtgagttgagatagagccactgcactccagcctgggccacagatcgagactccgtctcaaaaacaaaacaaaaaacaactttatctccaggcagggcgtggtggctcacgcctgtaatcccagcactttgggattctgaggcaggtggattacctgaggtcaggagttcaggaccagcctggccgacatggtgaaaccccgtctctactaaaaaatacaaaaattagccaggcatggtggcgggcgcctttaatcgcagctactcgggaggctgagacaggagaatcgcttgaacccgggaggcggaggttgcagtgagctgaaagcctgggtgacaagagcaaaactcggtctcaaaaaaaataaaaaataaaacaaaacaactggccaggcgcggtggctcacgcccgtaatcccagcactttgggaggctgaggcgggtggatcacgaggtcaggagatcaagaccatcctggctaacacggtgaaaccctgtctctactaaaaatacaaaaaattagccaggtggggtggcgggcgcctgtagtcccagctactcattctcctggaggctgaggcaggagaatggcgtgaacccgggaggcggagcttgcagtgagctgagatagcgccactgcagtccggcctgggcgacagagcgagactccgtctcaaaacaaaaaaaaaacacaaaaaaaacacaaaactttatctcctacttgaacctggggtactgcatttcctcttgcgcacagcaggcatcttaacccgtgagagacaatgtgtgttcatccttgtggaggcagttgagagcatatttcatttcttttcttccttttttttttttgagacggagtcttgctctgtcacatcacctaggctggaataaagtggcgtgatcttggctcactgcaacctccgcctcccaggttcaagcaattctcctgcctcagcctcccgagtagctgggatcacaggcacttgccaccacgccaggctaattttttgcatttttagtagagatggggtttcaccatgttggccagcctaatcttgaactcttgatctcaggtgatccgcccgcgtcagcctcccaaagtgttgagattacaggcgtgagccactgcgcctggcctttttttttttttcttctgagacagagtctcactctgttatccaggctggagtgcagtggtgggatcttggctcactgcagcctcgaccagcctccctggctcaagcaatcctcccacctcagcatcctgagtagctaggaccacaggcgcacgccatcactcctggctaatatatgtatatattttttgagaccgagtcttgctctgttgccgaagctggagtgcagtggtatgatctcggctcactgcaacctccgcctcctgggttcaagtgattctcctgtcttgccctcccgagtagctgggactacaggcgcatgccaccacacccggctgattttttttatttttagtcgagatggggtttcaccatgttagccagggtggtctccatctcctgaccttgtggtctgcccgcctcggcctcccaaagtgctgggattacaggcgtgagccaccgcacccggctaattttttttgaggtggagtttcgctcctttgcccagtgtggagtgaagtggtgcgatcttggctcactgcaacctctgccccttgggttcaagtgattctcctgcctcagcctcccaagtggctgggattacaggcatgcaccaccacacctggctaatttttgtatttttagtagagatggggttttgccatgttgcccaggctgatctcgaactcctgacctcaggtgatccactctccttggcctcccaaaatgctaggattacaggcacgagccactgcgcaccgccctgatttttgtattttttgtagaggtggggtttctccatgtttcccaggctggtctcgaacttttgagctcaagcaatctgccttcctcagcctccgaaagtgctgggattacaagcctgagccaccatgcctggccttttttttaaaattaattaattaattagtttttttttttttttgagacggagtctcgctctgtcacccaggctgcatggagtgcagtggcatgatctcgcctcactgcaagctccgcctcccaggttcatgccattctcctgtctcagcctcccgagtagctgggactacaggcgcccgccaccgcgcccagctaatttttttgtatttttagtagagacggggtttcactgtgttaaccaggatggtctcgatctactgacctcttgatctgcctgcctcggcctcccgaagtgctgggattacagacgtgagccaccgtgcccggttttttttttttaagatggagtttcgcttttgttacccagattcgagtgcaatggtgcgatattggttcactgcaacctccacctcctgggttcaagtgattctcctgcctcagcctcccgagtagctgggattacaggcatgcgtcagcatgcctggctaattttgtatttttagtagagacaggttttctccatgttggtcaggctggtatcaaactcccgacctcaagtgatccgcctgcttcggccttccaaagtgctgggattacaggcgtgagccacctcacctggccagcttttttttttttttttttgagatggagtctcactctgttgcccaggctggagtgcatggcgcgatcttggctcctgcaacctccacctcctgggttcatgtgattctcctgcttcagcctcccaagtagctgggattacaggtatgagccaccgtgcccagccagcaatttctataaataggaatttagagtagcttacaaaattacaccagccagaagacttaatttagccttaaggaatagggttctctgttttccaatttcttaggtttgatagctaagcattttctttttcttttttttttttttgagacggagtctgctctgtcacccaggctggagtgtggagtgcagtggtgcgatcctggctcactgtaagctccgcctcctgggttcatgacattctcctgcctcggcctcccgagtagctgggactacaggtgcccgccaccacacctggctaatatatgtatatatatattttttgtatttttagtagagacggggtttcactgtgttaggatggtcttgatctcctgacctcatgatctgcccaccttggcctcccaaggtgctgggattacaggcatgagccactgcacccggccatagctaagcattttcttagttttactcagggtccagggtagaaagatggatgagatgcattagtagaagatcataaactgagggtgtcaaagttggtgaagctggtctgtgttctgtgttgaagtggttaaatctgaacgaactttagcagtatgttgacttaatttatatttatgttttaaaagcaggattctactggaacaatcagctgcatatttaaaaaattacttttatgaccaaatgtcttttatcttgcttgtcatgagatacatttaatggtgttgaggttttgggctgggcgcagtggctcacacctgtaatcccagtactttgggaggccgagccgtgtggatcacctgagctcaggagttcgagaccagcccgtccaacatggtgaaaccccttctttactaaaaaatacaaaaattagctgggcgtggtggcgcacacctgtaatccaagctgcttggtaggctgaggcaggaaaattgcctgaacctgggaggtggaggttgcagtgagccaagtttgaggttttgcagtgttgaggtttttcttctcagttatacctttttgagctaatcgttttaaaaaataccactgtctaaaatacactgaaattaattttactatttataatttttggttctggattgactgtacagaaaatgtttcattttcttactgtgacctttgacaaagaagggtgagtaatttgtggtgtgggcaggagactcagtggcattccccacttcctctggagttggtcagtgtggtataattctatttcagttgtgggcattcaccaatattgtttcctccttttcctgcagggccactgctctccaggaagataaggcttttaaagcagcctagcactagaaggtgaggtaaggtacttgaaaagagacatttctgcaaaagaaagttgaggaataatcacatactggccaaaatgataaccacagaattatatttatttatttttatttatttttttgagacaaagtcttgctctgtcacccaggctggagtgcaatggtgtgatcttggctcactgcaacctttgcctcccgggttcaagcaattctcctgcctcagcctcccaagtagctgggattacaggcatgtgcccccgcgcccagctaattttgtatttttagtagagacggggtttctccatgttggtcgggctggtctcgaactcccgacgtcaggtgatctgcccgcctcggcctcccaaagtgctgggattacaggggtgagccaccgctgcccagcttattcgtttttgagacaggatctctgtctgtcacccatgctggagtgcagtggcatgattacagctcactgcagaccttgacctcctaggctcaagccatcctcccaccttggcctcccatgtagctgggactacaggggcatgtcaccatgcccggctaatttttgtaactttttgtggagatgggaactcactatgttgtctgggctgatctcaaacccctgggctcaaacgatcctcccgcctcagcctctcaaagtgctgtgattaatagacatgagcctccgcacctgacttagaattttaaaacattaatagtggaggaaaagctctattggataatacatacatttaagtagtgtggaagataaaagtggaaagaggctgggtgcggtggctcacacctataatcccagcactttgggaggccgatgtgcacagatcaactgaggtcaggagtctgagaccagcctggccaacatggcgaaaccctgtctctgataaaaatacaaaaattagctgggcgtggtggcaggtgcctgtaatcccagctacttgggaggctgagaaaagagaatcacttgaacatgggaggtggaggttgcagtgagctgagatcctgccatcacactccagcctgggcaacaagagtgaaactgcatctcaaaaaaaaagtggaaagaaaaggactgggcgtggtggctcacacctgtaatcccagcactttgggaggccgaggcgggtacatcattttaggtcaggagtccaagaccagcctggtcgacatggtaaaaccctgtctctagtaaaaatacaaaaattagccaggcgtggtggtgtgcgcctgtaatcccagctacctgggaggctgaggcaggagaattgtttgaatcttgaatctgggaggcagaggttgcagtgagccgagatcgtgctgttgcactccatcctgggcgatggagtgagactctgtctcaaaaaaaaaaaaaaaagaaaaaaaagtggaaagaaaagagaaaaggagcccgatctcatgcctgtaatcccagcattttgggaggctaaggtgggaggattacttgaagccaggagtttgagaccagcttgggcaacaaagagagaccctgtctctacaaaaaataaaaaaaaaaaaaaaaattagttgagtgtggtggcacgttccctaggtactcgggaagctgaggtgggggcatcttttgagcccaagagttttttttttttttttttttttttaaattttttgtagagagggttttgccatgttgcccacgctggccttgaactgctgagcttaagcgattcatctgcctgagcctcacaaactgctggattacagatgtgagccactgggccttagcctaggagttgaggctgcagtgagctttcatcacagcaaatcacaccactgcactccagactgggtgacaaagtgagaccctggctcaaaaaataaaaaaatgaaaagtctcttgtcgtcccctcccttttttttttttttttttttttttttttggatacagaatcttgcactgttgcctgggctggagtgcaatggcgtaatctcggctcattgcaacctctgcctcccgggttcaagcgattctcctgcctcagcctcttgagtagctgggattacaggtgcccaccatcacgccgagctaagtttttgtattttcagtagagacgaggttttactatgttagccaggctggtctcctgaccttgtgatcctcccacctcagcctcccaaagtgctgggattacaggcgtgaggcatcccattttattttttctgtaaatagggaatacaacacatggtactaacttgattaaaagttcaaaagggttatacaaaggagggaaagaaaccttccttccatctatcccctcagcctgttgttcacctttccagagacagttgtgatcccccctgattcctggggtctcctccagagatggcccttggcagtgttcttaccaagtttgagtatagatttttttttttttttgagacagagtctctctctgttgcccaggctggagtgcagtggctcgatctctgctcactgcaacctccacctcctggattcaagtgattctcctatctcagcctcctgagtagctgagacgacaagtgcgtgccaccacacccagctaatttttatatattcagtagagactgggtttcaccatgttggccaagctggtctcgaactgaactcaggtgatcccgtccgtcttgatctcccaaagtgctgggattacaggcgtgggattcaggcccctgcgccggcctgaatataacatttctttttttttttttttttttttttttttgagaccgagtctccctctgtcacctgggctggagtgcagtggctcgatctctgctcactgcaacctccgcctcctgggttcaattgattctcctgcctcagcatcctgagtagctggcattacaggcgcgcaccaccacacccggctaatttttttttgtatttttagtagagacggggtttcagtgtgttggtcaggctggtctcgaactcctgacctcgtgatccacctgcctcggccccccagagtgctgggattacaggcgtgagcccccgcgccagcctgaatataacattctttttttttttttttgagactgagtctcgctctgtcacccaggctggagtgcagtggctcgatctctgctcactgcaacctctgcctcccgggttcaatcgattctcccgcctcagcatcctgagtagctgggattacaggtgcgcaccaccacacccggctattttttttttttttttttagtagaggctgggtttcagtatgttggtcaggctggtctcgaactcctgacctcgtaatccacctgccttggccccccaaagtgctgggattacaggcgtgagccactgtgcctggtcatgaatataacatttcttttttttttttttttttttgggagacggacggagtctcgctctatcgcccaggctggagtgcagtggcgcgatctcggctcactgcaagttccgtctcctgggttcacaccattctcctgcctcagcctcccgagtagctgggactacaggtgcctgccaccacgcccggctaattttttgtatttttttagtagagacggggtttcactgtgttagccaggagacctgttagccagaaaggtctcgatctcctgacctcgtgatccgcccgccttggcctcccaaagagctgggattacaggcgtgagccactgcgtccggcgcatataacatttctaatggttaatctgtccctaattcttagacagtttggttctttttttttgtttttcttcttattatttgttttagctatttcctacaggaaggaggttatttcttactttggttgtaatagataatgttgcaattaacatctctgcataaagttttttttttttttttttttttgagacggagtctcgctctgtcacccaggctggagtgcagtggcacgatctcggctcactgcaagctccgcctcccgggttcacgccattcttctgcctcagcctcccgagtagctgggactacagttgcctgtagccacacctggataattttttgtatttttagtagagagggggtttcaccctgttagccaggacggtctcgatctcctgacctcgtgatccgcccgcttcggcctcccaaagtgctgggattacaggcgtgagccaccacgcccggcctgcataaagttttttaaaagttttttgtatcataagaagtgaattgtggccgggcgcagtggcttacgcctgtaatcccagcactttgggaggctgaggcgggcggatcacaaggtcaggagatggagaccgtcctggctaacatggtgaaaccccgtctctactaaaaatacaaaaaaaaaaaaaattagccgggcgtagtgttaggcgcctgtagtcccagctactctggaggccgaggcaggagaatggcgtgaacctggaaggcagagcttgcagtgagctgagatagtgccattgcactccagcctgggcgacagagtgagactctgcctcaaaaaaaaaaaaaaaaaaaagtgaattgtgatttacagttttgtagaagcacatttatcaatgtcaagtaaggcagatttgtactgatttttatttttaagtattgtgcacgttttctgtagtcccttttctgtgacttgcatggatttgtgatttttatagccatgtggagacctagtctcagtgttccctgcccttggggacatgaggagccacagattcattcttgctgaggcagtcatggcccttggataccctaagtataggggagtgtgccagttgtggggagctcaaggatgaatgaagcacttggaaggatatgtcgtattcagggaaacagtagccagtgcccagcttgattccctgttttggcatttactcttcatttctatagatgttgtttttgtatatgaggtatgagaatctacaaacaagcacctcttatagagttctttctcctaatcggtacacagttttctgtatctataggttccacatctgtggattcctccaactatggattgaaaatatttgggaaaaaaggatggttgcgtccgtatgaaacatgaataaacttttatcatcgtcatcatttcctaaatgttagaacaactattttcataccatttacattgtattatgtgttataagtaatccagggatgtttaaagtatacaggaagatgtttaagggatccagggctgtgcaggacgtgccttgttaacaaaatgtttacaagcagtatacttggtaaaagtcatcgctattctctagtctcaataaactggggacacagtgcattgcggaaaaccacagggacctctgctcttgaaagcggggtattgtccaaggtttctccccatgtgatagtctgaaatatagcctcgtggaatgagaaagacctgaccttcccccagcccgacacctgtaaagggtctatgctgaggtggattagtaaaagaggaaagccgcttgcagttgagatagaggaaggccactgtctcctgcctgcccttgggaactgaatgtctccgtgtaaaacctgattgtacatttgttcaattctgagataggagaaaaactgccctatggggggaggcgagacatgtttgcagcaatgctgccttgttattctttactccactgagatgtttgggtggagagaaacataaatctggcttatgtgtacgtccatccagtcacagtaccttcccttaaacttaattatgacgtagattctattgcttacatgtttttgtccttattatgatgcaaatattacaccattttatataagggacttgagcacttgtggattttgatatcctggaaccaatccctcatggataccgagggaagagtttgtaaatgtattaaaacaaagtagtggaattggaatcaaattcttttgattttgtaggtatctgtgtttagccttttcactgtgctgtgaagatttcccccccaggcatcctaggggttgatgggagatttacttaagattcccaaggctgtctagggcagaagtgaaccagttctgccttggttgtcataaacacctctgttaattagctcagttgttctccagggatagattgattctctagctagcttttgccaagaaaaatgatccagtcaataatggaattccaaataagatttcctttttatttggctattaatcagaacctgggataacatccattttctgggccctgattttactatggaatatttccataaaacatgcatgtgattactgtaaatgtcctgtaaacactcctgcttgggctggtaggtcatctttttttttttttttttcttttgagacagttttagtcttgttgcccaggctggagtgcagtggcgtgatctcagctcactgcaacctctgcctcccagattcaagtgattctcctgccacagcctcccaagtagctggaatgatacaccatgcgccaccacacctggctaatttttgtatttttagtagagatggggattcttcatgttggtcaggctggtctcaaactgcgacctcagatgatctgcctgcctcggccttctaaagtgctgggattacaggcgtgagccaccatgcctggactgtaggtcacctctgtgtccaatctgccagaggtcagggatacgttgccgtctaggattttttatattggagaccagtctgatgagtagcccttgagcagggactggattctaagtcttggagaaaatgagtaagttctacctcttatgtgaggtatgattgaagaaattgtgttttctttgttttttgagaggcagggtcttgccctgttgcctacctggagtgtggtggcataatcatagctcacttcagtcttgacctcctagggtcaagcaatcctcccacctcagcctcctgagtagctgggactactagcacgtgccactatgcccagctaatttttaaatttttttgtagcgatgtggtcttggctatgttgcccaggctggatcctcctgcctcagcctcccaaagtgttgggattacaggcgtgagccaccacacccggcccagaagtgtcttatatttttagacttttgagcagtgttggaacgattgaattgcctgagtggagaaatttggtaggaagagagtagaagagaggctctttattaaagacctaaaaatagaattcattaaagtcacaaatagataacagtaggattcctgtctgactctcttctgctttgggtttgtgctaccagctgttgcgtttgcttacctagaaaatagaataatattttgtcagaagggtcaacagtatggtcctaaaacattgtgtttgcaagtctgaggatgtgctttttccaattttaaaagtagttagaaagtgaccagattctttcatgtaggggagaaatcttagcaggagacgttggttcttctgcttgtagtgccagccctaacctgacaggtcctgtggggacccctgtctcttcagagccctgagcctttttcccttctagttggactttgctgactagagagggatatcctatgtaggccacctgattacctgatttaatcagatggtacctctgttattttggagaagagagtctccttttgattctaatagctgaagataaaaatggctccgtatgctttcttcctgtggagggctctgctgccctgagctgcctgtgccctttacatctgtgtgagggggtggaggaatgtcctggcctcctcacttccatcctgctatgggcttccaggaagaatggggtccattgtgggctgtacccaaaggcacagtcctctgcctcagtgtcaggcttactgtggtctctgtgcccagtttgctgtggtcactgggtgccaaggggttgggagctcaggaagccattgctgcttttttttttttttttgagacggagtcttgctcttgtcacccagcctggagtgcaatggcacgatctctgctcactgcaacctccgcctcccaggttcaagcaattctcctgcctcagcctcccgagtagctgggattacagatgcgcgccaccacattgggctaatttttgtatttttattgttattattttattttattttatttgagacggagtctcgctctgtcgcccaggctggagtgcagtagcttaatctcggctcactgcaagctccgcctcccgggttcatgccattctcctgcctcagcctccggagtagctgggactacaggcatctgccaccacgcctggctaatttttttgtatttttagtagagatggggtttcaccgtggtcttgatctcctgacctcgtgatctgcccgcctcaggctcccaaagtgctgggattacaggcgtgagccaccgcacccggccaaggacggagtgttgttctgtcgcccaggctggagtgcagtggtgcgatcttggctcactgcaaccttttggagacaaaaggcctttgttgtgtctggcctttatctttgttgtaatgaccctgaaaccgtggcctgagtctttgaggtggaagttgccctagaaatgtcatgtaatataccagaatttatcaagcctttctctagtactgggacacttaacatgtgcctgtggtacttttgtttgttttgctattatttcacatttaatttcatttttgtaaataaataattttgaagccaaattcagatttttcatctttatgtctacttaggttgcttagacctaatgtggtagatcaggttttgtttttgttttctttcttttttttttttttttttttttgagacaaggtctcactctgtcacccaagctagagtgcagtggcatgatcatggctcactgcagtctgaattcctgggcttaagcaatcctcctgcctcagccttctgcaaagctggaactacaggcatgtgccaccatgccagcctatttttttttccttttgtagatacatggtcttgttatgttgcccaggctagtctcaaactcctagcctcaggtgatcctctcatctcaaccttccaaagtggtgggattacaggtgtgaaccaccatgcccagcctgttttcttgttttgaatccaagctttctgagggttgtcctttaggaggagaatgtaaggaattccagagtcagggaggcatgggctgtgctctgggagaggcctggggaggcactgctctggaactgcctgtaggagtggtgtcagactgccagcagaagccagggtggtgaggggagggctgaatgttctgcaagagagatttataccactattttctttttcttttatttatttatttatttatttatttatttatttatttatttattttttgagacggagtcttgctctgtcgcccagactggagtgcagtggtactatctcggctcactgcaagctccgcctcccaggttcacgccattctcctgcctcagcctccctggtagctgggactataggcgcatgccaccacgcccggctaattttttgtatttttagtagagacagggtttcaccgtgttagccaggatggtctcgatctcctgaccttgtgatcctcctgcctcagtatcctgaagtgctgggattacaggcgtgagccaccgcgcccggccctttttcttttttttgagacagagtctcactctggagtgtagtggcatgatctcggctcactgcaagctccacctcgtgggttcaagtgattctcctaactcagcctcccaagtagctgggattacaggtgtgcatcaccacgcctggctaatttttatatttttagtagagacagggtttcaccctgttggccaggctggtctcgaactcctgacctcgggtgatctgcccacctcagcctcccaaagtgttgggattacaggcgtgagacactgcgctcagcctgatatgaatattcttgacggtgtcttctgggtatatacctaagagtgaactgttagatatatagtatttgattttttttgtttgttttgttttgctttgtttttgagattcagtctcgctctgttgcccaggctggagtgcagtggtgtgatcttggctcactggatcctctgcctcccaggttgaagtgattttcctgtctcagcctaaccagtagctgggattacaggcgtgtgccatgcctggctaatttttgtatttttagtagtaatggggtttctctgtgttgcccagactgatctcgaactcctggattgctcaagcaatccacctgccttggcctcccaaagtgctgggattacaggtatgagccaccgtgcccggcctgatgttttaattattgagaattttgtattttttgtagagatggggttcttcctctttgcctaggcacacgactgtctatcccagatggctgcactggcctaggacagacggttcttcccccagtacgtcaagaccctggagggtgcaagctacagaacagctgggctcctgcattcctcccagaccctgtcttcagtaggacaacaggcaacaaacttaaatttggtaatatgaatttagaaaatgttaatgttttccagaacagtgaaactacaatataaaggttagacttgagaaccaaagttactctctagcttccctcttctgcccttcatgtgctcagcacttgtatctgtgttctcactacccctgagccctgagttcttctggggttggcccaggaggtgccttggttcagagagatacctgggaggaaaccacatggaatcaacaacagtactacatatttgttattatttggagtaattagccccatacattctttttttttttttttttttttgtttgagatgggatcgtgctctgtcacccaggctggagtgcagtggcgtgatctcggctcactgcaacctccgcctcccgggttcaagcgattctcctgcctcggcctccccattagctgggactacaacaggctcccgccaccatacacggctaattttttgtgtttttcatagagacggggtttcaccatgttagccaggatggtctcgatctcctgacctcgtgatctgcccgcctcggcttcccaaagtgctgggaattcagacgtgagccaccacgcccggccagccccataaattcttgatagcaattttcctgaggtactcaagttttactagaaaaacatagcatcatgttttgattcattagttgtctagatgctcaccctcctgtattatcattctggctaactattaagtaccattactcccagattgaatttatgacacctgctgcccaccatgcccattaatcaaacaataagggcagggcaaactgctgtctcatgttgtttcaaactgccccacctccttcctcttccatcttatgttattaatgaaagagtttgacaatacttaaatgttagtgaaaataagagaaaagtcgtttgtatctttaggtgagggtgtaaattgaaggacattttgccaaatctaacaattccaatgtacatatcctttgacccagcaattccacatctaggattatatccttagatatgttctaagaagaatgcaaggatgagtgtgagaacaccctggagagcaagatatgcaagatacttagacatttagatgtgccacacacaataggggtgaatgagaagaaggatctacccactggggctgaggtctctcagttctgctaaattagaaaaaggaacggttatttatttatttttttttgatagggattctcattctgtctcccatgctggagtgcagtggcgcggtcttggctcactgaaacctttgcttcccagggtcaagcgattcccctgcctcagcttcctgagtagctgggattacaggtgcgtgccactatgaccatctaattttttatatttttaatagagacggggtttcaccatgttggccaggctggccttgaactcctgaccttatgatctgcttgccaggtgtgagccactgcgcccggcaaaaaaggaacagttttgaagaatttgatttcatttgaactttaagaatgaaattaaaagagatgtgcttgtttatgtgtttagagatatacattcaatgtgtcatttgtggagagctactgaagaaactcagtctgctcacctgaggacctgggaaggagacctgcttttcatttgtgtcctagtgtggtgttggaatttattttacctccatgtgaattaccttttttttttttttgagacacagttttgctcttgttgcccaggctggagtgcaatggcgtgatcttggctcaccgcaacctctgcctcccggattcaagcgattctgcctcagcctcccgaatagctgggattacaggcatgtgccaccacgcctggctaattttgtattttcagtagaaaaggggtttctccatgttggtcgggctggtctcgaactcccgacctcaggtgatctgcccacctctgcctccagtagtgctgggattacaggtgtgagccaccgtgcctggcccttgaattacttttattgcattaagtcaaaggggaggaccagatagttgatgacatcattgtgagatagactgggaaccaatagtagggaatcggatactgggaaacactggatgcagcgtgcccacattttgcccttagctctgggacctcttaacttggtaatgatctgatttgggttttttttcctctaggagggaaggttcctgggaagggaagatagaatctatttttaatcttaagaaaacatagctcaatgtagtaattttgggccctagtgtgacattttcttccattataacccaagtcacttttaaaacaaaatcctttttttgtatattttgtgtttgtaatgtttctagaggaaaatgcttttaaagtatcctcacttcccttcgtaaacttgggagggcttgggacctggtggggtatgtgttttgaatcatgctgtctgggggattagtgcctggaatccagattcctactggacaaagcgctggggtttgaggtaagtggggtggggtagggggagggttatatagctgtcacagattacagagcttacctgctgactgcttcacaattgttggtacatacacctgctttggcatggacgtcctgagagggggcttggaccatgacccgtgttcctttctcttaccacttgtttttttccattttggctgggcgcggtggcttacgcctgtaatcccagcactttgggaggccgaggcagacggatcacgaggtcaggagatcgagactatcctggctaacacggtgaaaccccgtctctactaaaaatagaaaaaattactgaggtgtggtggcaggtacctgtagtcccagctacttgggcggctgaggcaggagaatgatgtggacccgggaggtggagcttgcagtgagccgagatcgcgccactgccctctagcctgggcgacagagtgagactctgtctcaaaaaaaaaaaagttttttttttttatataactttaaatttacagaaaaattgcaagaataattcaagaaactcccaaatactccatctagagtcaccatttgtttgcattttgctgtgtttgctctgtcattctcttttctatctatacctttttttctgaaccatctatgagtaattgacagaaatcatgcctctttattcctaatttttttattcttttattttttttttttgggacagggtcttgctctgtcacctaggttggagtgcagtgatgagatcttggcttattgccacctccgcctcccaagcttgagcgatcttcctgccttagcctcccgagtagctgagactgcaggtgcaggctaccatgctggcctaattttgtatttttttgtagagatggggtttcaccatgttgcccaggctggtctcaaactcatcagtgcaagctatctgcccttctcagcctcccaaagtgctgggattacagacatgagccacaacctggcctactcctaaattctttttttttttttttttttttttgagacggagtctcactctgttgtcttggttggagtgcagtggcacgatcttggctcactgcaacttctgcctcccaggttcaagcgattttcctgccacaatctccctgagtagctgggactacaggcgcgcactactacgcctggctaatttttgtactttagtagagacaggtttcgccatgttggccaggctggtctcgaactcctgacccaggtggtccgcctgcctcagccttccaaagtacaggcgtgagccaccacgcctggcatttttttctatttttaatagagatggggtcccactatgttgccggggctggtctcaaactcctgggtttaagtgatcctcctgcctcagcctcccgaaatgctgggattacaggtatgagccaccgctcccagccaagttgttttttttttttttgagacagagtctcattctttcgcccaggttggaatgcagtggcgtgtttttggctcactgcagcctctgcctcctgggtcaagcagttctcctgcctcagcctcccaagtagcagggatcataggcacctgccactatgtcctgctattttttttttcttttttttgagacggagtcttgctatgttgcccaggctggagtgcagtggcgcgatcttgctcactgcaagctctgcctcccgggttcacgccattctcctgcctcagcctcccaagtagctgggactgcaggcgtccgccaacacgcctggctaattttttgtatttttagtagagatgtggtttcactgtgttagtcaggatggtctcgatctcctgacctcgtgatccgcccgcgtcggcctcccaaagtgctaggattacaggcgtgagccaccgcgcccggcctgtcctgctatttttgtatttttagtagagacggggtttcgccacattggccagtctggtctcaaactcctgacctcaagtgatcctcccgccttggcctcccagagtactgggattacaggcgtgagctgcctcgcctggccccaagtctattcttttttttttttttgagacggagtctcactttgttgcccaggctggagtgcagtggtgagatctcggctccctgcgacctctgcctcccgggttcaagcaattcttctgcctcagcctcctgaataactgggattataggcatgggccaccaagcccagctaatttttgtatttttagtagagacagggtttcaccatgttggtcaggctgttctcgaactcctggccttgtgatctgcctgccttggcctcctaaagtgctgggattacaggcgtgagccaccgtaccccgcctggctccaagtctattcttaagaccacagaacttttgatttaagattcactggacagatacataacaatacataaaataaatgttttatttacaaaataagacctttaagtagttagctgccagttgaaataatgatttggactggatgtggtggtttatgcctgtaatcccagcattttaggaggctgaggtgggagaattgcttgagcctaggaatttgagaccagcctgggcaacgtggtaaggccccatctctacaaaagataaaaaattagccaagtgtggtggtgccatgtttgtaatcccagctatttgggaggctgaggtaggaggatcaattgagcctgggagggtgaggctgcagtgagccatcatcgtgtcattgcactccagcccaggtaacagagtgagaccctgcatctttaaaggaaaatttaaaaagagataaatgcaggagatcttccatcttatcaaaagggtgctgttataatttatatatttttaagtactggatggggaggtggagtagcagctccaaggtacttttcagtggtggccttaatctgctgatgcatggaaggtgtgttcttcttccagatagccctgggatctcctgaatttccaggttgggctactcaggcatggattgactcctacagttcaggcggatggagatcccaccaacccacagcttaattcttcagatgctttatcttcatttctcatgtatcttcagttggcttctaatgtgagcacataaatgccctgcagaatttctttcccatctttgtgcaaatcattatttttattctttttttttttttttttttttttgagacgacagggtcttgctcagtcacccaggcgggagtgcagtggcatgatcatggctcactgcaacctcaacctcccaggctcaagcgatcctcccacctcagtagctgagtagctcagattataggcatgcgtcaccacactcagccaattttttcatattttgtagagacagagtctccctgtgttgcccaggctgatttcaaactcctgggctcaagcaatccatcaaccttagcctaccaaagtgctaggattacaggcatgaaccactgtgccttgcctgcttttcatttttaatccttgggttctaaaatgcaagaattaatttccaaataattagtatggaatggaatcccactttttcctgtgtgctgagagaatgatcaaagatcacagaaaagagatggagacagaagaagctgagagcaagggcatctttaaggatttcttcagtcttgttaatctgggtgtaaattctgtgtggctgacagcctctgaggatcccttgaagttttgaggaggaggaaagcgcttgagagtaatggatttacttgattttactttgtatagaagagcctgaactggacatatctccaggtatgtgctgtggtctttaacaagcagcccaacatcctttgctggggaaggccagcaatgtgggagctagtggggcctagcccaggagccttgcgttcaccaggtcactgggaactggtgctttctgatgcacctctatgcaagcctgattctgaagtcagatgtggtgggcatccctgtccatagtgttggcctggcccaccatttcactggtcatagaatggctgtagctttgcagagcagggattgcgagaatggaagaatgtcttttggctcttctgctctagcttttggaagtacgtcagagaagtttgtgattttttagaatttgtagctgtggcaaggctgaagcaggtaaaagggccaattagaaccatctgtgggagggaaggaattctgttagcaggaaggaaaacctgagcaaagggagtgggtggtagttttgtcttgttcttttataaacacagtgtgttgatatggcagttcctggcatgggtgaaatatttttgatggttgagcattaagaatgtagccaaaaacaagaatgtggctaaaagcaagtcaccactttaaacactgaagattgaagcatttgcctagataaatattctgcagtgaatatgccagaattcccacatacctgtttggctcctgaggcattgtgcttagggtcccaggcctttattacaggggctgctggcagggtcaagggctgggttgcctggagcttccctgacatttcgcctcactcctctgttttggagtctaattggccaattaggtcagaaaggccaccagccagcacactttttctaaatttaattcatcattgctagattagcatattaattttgagctatggcacttatttttaatctttcgtagtgtaatggaaaaaataccagaaagctcagcattgaacttcaaagtgtgaaatcaagtgatcattgcctttatgcttggtgaattattattattatttttttttttttggagacagagtcttgcactgtcatccaggctggagtgcagtggcgtgatcttggctcactgcaacctccgcctcacaggttcaaacgattctcctgcctcagcttcccgagtagctgggactacaggtgcctgccaccacgcccagctaatttttgtatttttagtagagacggggtttcaccatgttagccaggatggtctcgatctcttgaccttgtgatccacccgcctcagcctcccaaagtgctgggattacaggtgtgagccaccggcgcttggctgcttggggaattatttaggggatttagtggaatttcctttcctgtactctgtgggcatctgctgagtacctgcttggtgccaggcatctactgggagagaaagcagcagagtcaatgctgtcaagacccctgcagtggacctcttctcccccttttctctcaagggaaccattggcatccagccttgacccccagtagacactgatcatccagcccccacagggtagccccactgtacctttgtactgagttggtgaggggatgggatagccagggggagtttggggaagtcagaaggcaactttcagtttctattagagtaagcacttggaaaatgtctgtttataatgtattagtataaatacttggtttgttaccacttgtgtttcctgatttagtaatgtatataaatcttaattgaatatttttgtgtgaaacttggaaagactgcatctatagcaaataaagcctcacacctagcacagggtaggtagttggtaaacatgtgctgaagtgttaattgttgggctgtcttcagagaattgtagtttgctttaattgcattttttgatgcaacgcgggtgcaagggtgtggtaattcattcccacttggattctgctgaggctggccacccacctctcagacttattcccccaccacctctccacctcctactctagctgctgcagccaaatggacagttagggccgggcacagtggctcatgcctgtaatcccagcactttgggaggctgaggcgagcggatcacaatgtcaggagttcgagaccagcctggccaatacggtgaaaccctgtctgtactacaaatacaaaaattagctgggcgtggtggcaggcacctgtaaccccagctactcaggaggctgaggcaggagaattgcttgaacccggaaggcagagtttgcagtgagccgagatcgtgccacttgcactccagcctgggcgacagagcgagactccgtctcaaaaaaacaaaaacaaaaacaaaaaaacagaaaaaaaaaaaccccaaatggatggtttggcctggtccttatctttggcctcagtttatccttttctgtcttcagtgtcttacctgggcactggtgattaaagtactggaccatgtcatcacggggatatgcccctaaaagcctcctaaagtattttcttctgaagtctaattggtgtaactaagtaatgttttctaaaatattgtggcaaaatatacataacataaagatggtgtatttatttatttattttgctttgtcacccaggctggagcacagtggtggagtcatggctcatggcagcctcaacctcccaggctcaagcagtcctcccacctcaggccaagtagctgggactacaggtccatgcctacgtacttggccaattttttaaatttttaacttttttttttttttttttttttgagatggagtctcactctgtcgcccagactggagtgcagtggcgcgatctcggctcaccacaacctccacctcccaggttcaagcaattctgctgccttagcctcctgagtagctgggattacaggcgtgtgccactgcgcctggctaatttttttttgtatttttagtagagacagggtttcaccatattggccaggctggtcttgaactcctgaccttgtgatccgcctgccttggcctcccagagtgctgggattacaggcatgagccactgtgcccggcctaaatttttttttctttttttttttggcacaatctcggctcactgcaacctctacctcccaagttcaagcagttctcctgtgtcagcctcctgagtagctgggactacaggcatgcaccacaacacccagctaatttttgtatttttagtagagatggggtttcaccatgttggccaggatggtctccatctcttgatctcgtgatccacccacctcggcctcccaaagtgttgggattataggtgtgagccactatgcctggcctaaatttttttttgtagagatgggggtctcactatgttgtccaggcttatattttaatataccatcttaaccattaaaaatgttttttttaggggctggctgtagtggcttgtccctatagtcccagtgctagaggaggctgaggcaggaggattgtttgaaccaggagtttgagaccagcctgggcaacatagtgagaccctgtctctacaaaataaaaactaaaaaatggccgagcatgatggttcacacctataatcccagcactttgggaggccgaggtgggtagatcacctgagatcaggagttcaagaccagcctggctaacatggtgaaatcccatccctactaaaaatacaaaaattagctggacgtcatagtgggcacctataatcccagctactggagaggttgaggcgagagaagctcttgaacccaggaggcggaggttgcagtgagccgagattgtgctgatgcactccagcctgggtgacagagcaagactccgtctcaaaaaaaaaaaaaaaaaaaattaaaaaatgagccaggcatggtgctgtgaaccagtagtcccagttgtgggaggctgaggcagaaggatcacttgagcccaggagtttgaggttacagtgagctattactgtaccactgtactccaccctgggggatagattgagagactgtctctcaaaaaaaaaataaaaataaccccaaacccacctttttaaattgtgaaaaacacaatataaaacttaaccatcttcaaagtcaagtatactgttcggtaataaataatgtttaacgcagtgtatggacttcagtggaatgtagaaggaattctttcagtgagccccattacgtaccatgttaaaatctactcagattaaaagccatggttcagtggagaatgactgttctgggttttgaaccttgcttttcttccctttccctagaacatgtctgcacctgggtggatttatacctaggttttaaacctttgcttctgtctttgttggaaaaaaccaaaccaccgccagcagaagccactgccctttgtttatctgtgataagtgagaatttgtaagaattttccaagtgagagaagtgttcaaagtagcttttatgtttgatagaatgtgtagtgcttgcctctcaggggtggtattttttctcccacgtatttttacctttaaatctcttgctgtatatgtgaccctggctccctttggttggtcctccagctccctgatggtcctggggctccctccatatcactggtggtatttccacattgcctgcagtttaggtaggaacctgtcccatgtcagggaaaccctatctgtcccctgaactgagcacttttaattttagggtgataccttgaatcttcaccagtcacttgacttcagacctttctcatctgacgccaaggagtcaaaaaggcctgtttttaactagaagcagagacatttgccatttttttttttaatatttggagctcaaataaacaatgacttcacaccactgttttccactgtgttgagtacatcctgttggccatgatattgagcagttttaaactggttaacctttcagaaactttatattaatgttggactgacagttaatatttaacttgtcaaagtgatttttgctagattgaacctgaagtggttaatatttctgctttttatgttttccaaaaccatttaaaataggaacatggtattttattattttaatctggtttcaaaatgaatatcccattttctttattattattattattaattaattttttttttttgagataggttcttgctgtgtcacccaggctggagtgcagtgttgtaatctcactgcaatctcaccatcctgggttcaagcagttcattcacctcagcctcccaagtagctgtgattacaggcatgcgccaccatgcctggctgaactttgtacttttttgtagaaacagggttttgccacgttgcccaggctggtcttgaagtcctcagctcaagcaatctgcccacctcggcctcccagagtactgggattctaggtgtgtgccactgcacctggccattccttaaatgttttaatgactgtggcaaatgatagcaatttaacagcttaactactatttttaggtagcgtagctatttgggagtaggggctcctaggagtggtgggatgggctacctctgccaaattttgtgtggttaataatgggttgagtctcccaacttttcttttattcttgggaagcaaaaactaatagagagctgttgtttcagagtttttaggttaagatgtgtgaaagtttcagagccgggtacagtggcctgtgcctgtagtctcctatttgggaggccgaggcaggaggatcactggggagttcaaggctggtgagctataattgtttctgtgaatagccactgcacactagcctgggcaacatagtgagaccctgtcttaaaactaaaaacagaggtacttaaaaatatatatatctgaaagtttcattcactgtgagtttttctatggatgaggaaacattaagtttatggcaaattgagttggtgccccatgtgcatttgttcttcacatctgttgagtggaaagcatttattaactgagttccctgtgtgcgtgggcactcatagctctggggaggcagcctgagtggaacagagccgcctgttccataaggctgtctggacagaatggggtcataagggcctggctcctcatgggcatctcccctttcctcccaccaagcaacatcctttggttgcatcttcctgggggatggtattggtggtggtgatgggaggggccagtgggatccttacctgctgaaccttacccttggatgcctggtgtccttgccagttactgtataatgtttttgatttcctagttatatccctgatgactgatctgataaactttaaatatacctgcctctagggtagctttaatatattttggctacaaaggtcactaccttttaggtattattgataacttgacattttacttcttcttctttttttttttttttgagacagaatcttgctctgtcgcccagtctggagtgcactggtgtgatcttggctcactgtagcctccgcttcccgggttcaagcaattttcctgtctcagcctccgcttcccgggttcaagcaattttcctgtctcagcctcccgagtagctgggattataggtgtccgccaccatgcccgactaatttttgtatttttagtagagatggggtttcaccatgttggccgggctggtcttgaactcctgacctcgtgatctgcccgcctcagcctccaaagtgctggtattacaggcgtgagccactgcacctacctggcctgacattttacttctgcccatcacttgagagtcccacatatcacaactgggaggaagggagtgcctggtcattggcagatgcgagccacttactggccctaccccaactagctctggttcttctggctgagggtgtttatctgcagtgagtctcagggtctggggtctggggcagtgctgagagcttagactcagttgtgggtactgcactgggtgctggcacagaggaataaggttcccattcccactactcgaaaaagagcttctgtagctcctgtcagccagtggtgaatccagctaagacatgttcttgggctgccttgagcacttcctactctcttttagaggatataatgtgaatgagtgtgaggtgttaaattgggtagctgtgtcaggaagactaggtggctgtggccagaatcttgccagaatctttctgtggttcccaagcactcttttctcccacaagttctggccaccaagctgtatgtgaccacgactgagtatttacctctgtttggaccttggtctctgaaccacgaggaggaggatctctgtagcgtctctatctgaatgactgagtgtatgagtatgagactgtgtaaaaaaaacttccagagggacattctgatacaagtatatggatgcagcctatcagattcctagcacaggcagacatgacccttgcttataggaagccagcatcagaaagacaaaagggtgttttattaccaagttgcaaaaaatttttctccttagagcatggaaaaccaacacatagtagttggagttgtgccctgaacatcgcctgcctgctaaatgcagagcacttagtcatgcccatgaaacagacaccccatgtcgtcatgtgtccgcagatagcagacagcttaagttcctggtagggcagggtgtgtctcataactgactgtttttaaataagctgagaattttaaaattgagaatgagattggaatagtgaaagatcagctggatctaataagtgaagtaaagactaaagatgatgggaagaacctgaaaaaaaggtcctgtgtctccccattcattcattaggtgtatggtgtatgtctggcactggatgagtgctgggcacatattagtgattgcaacagatgtatccctgctctcccacaacttatagtcttgtgggggtgttggttgagctgagccatttactaaattagaattaaaatgcgtctgggcatggtggctcatacctgtaatcccagcactttgggaggccgaggtgggcagatcacccgaggtcaggagttcaagaccagcctggccaacatggcgaaactctgtctccactaaaaaatacaaaaattagccaagcgtggtggcaggtgcttgtagtcccagctacttgggaggctgaggcagggagaattgcttgaacccgggaggcagaggttgcagtgagccgagatcatgccactgtactccagcccgggtgacagagtgagactccctctccaaaaaaaaaaaaaaggattaaaatgcataagaaaggaaaaggccagtaaagaaaacactatgcaacagtaaagaaaactgtaggccaagcacagtggctcacgcctgttcccaacactttgggaggccaaggcaggaggattgcttgagcccatgagtttgaaaccagcctgggtaatatagtctgtatctcttcaacaaaatttacaaattagcctggtgtggtggcgcacacctgtagttccagctacttgagaggctgataggaggttttcttgagcccagaggttgaggctgcagtgagctgtgattacaccatttccagcctgggtgaccaaaaaaccaaaacaaaaactacacagaaaagagttgaagaaaaaccttcaacttggagcactttatagagatctgaggagaagaccctcaccagagtactgggactgctggttgttgagaaggagttcctggtggaggacccctcagacatcactggaagttaaatggttcttggaactgatgcccatagggctcctccatctatgtacatggtcatttggtcccttttgctgagtgtggcacttaacattgctggtgagcctgtggtgggcaaaagtgacccagccatctactgatggcagggagggaaggttcctgcggaggaatgggtctgaaatcctgtgagccacctggtcccatgggccttggagtgtggaacaaggtagtagagaggaaagaggacaatgttttacctattacttatctcacattgatgacaggaatcaatcaaactatatcgtgctaaattgtaagacatctttaactttcttttttttttttttttttgagacggagtcttgctctgtcgcctaggctggagtgcagtggtgcgatctcggctcactacaagctccgcctcccgggttcacgccattctcctgcctcagcctcccgagtagctgggactacaggcgcccgccaccacacctggctaatttttgtatttttagtacagacggggtttcaccatgttggccaggctggtctcgaacttctgaccttgtgatctgcccgccttggcctcccaaagtgctgggattacaggcatgagtcaccgcgcccggccagcactttctgagatttacgcatgttgatacttgttttagtttttttaattgtcctacagagttccatgatattccatgaaagcctattgatatattttccagtacatggacattaggattgtttttaactttccagtgttgcagtgaacatgcgtatgccatctctccatatatgtttgcactttcctgtctgtgtgcctaggagtggaagggcccagctacatggtgagtcttctccagggtgctagggcagctcacagctcccaccaacagccatgtggtcctgttccctcagtggtgtcacattggaacattttggcccatctgattggtgtggaaaggtttttgctgttactttaattttcttttccctgattattggtaaggttgagcatcctttttgtaagtttactgctattcaggttttcttctctgtgaattacttgttggttcgttcttcctatcttcagcccatttttattgttttaggttgcttgatattttttgataaggattcattacctatcacatgcattgaaaaagctttttcccagcctgtggttagccatttttttctttctttaaggtgtcctttagtcattaaaaattattggccaggcctggtggcgcatgcttgtaatcccagaagtttgagaggccaaggtgggtggattgctttagcccaggattttgagaccaggctgggcaacatggtgacactgtctctacaggtacaaaaacatagccaggtgtggtagcacatgcctgtggtcccagctatttcggaagctgaggtgggaggatcgcgtgagcccgggaggtcgaggctgcagtgagctgtgattgtgccattgtactccagcctgggccacagagtgagaccctgtctcaaaaaaaaaaaaagaaaaaagaaatttaaaggtttttttttgagacagagtcttactgtgtcagatttaaaattcaaattcaaatttatcttaagatttaaaagattttttgagataattgtgaattcacagttgttagaagagggatcctatgtacagttacccagttccccccatggtaacatcttgcaaaactatatagtacaatatatatcacaggcaggatattggaaatggtatagtcaaggtacagaacatttccattgccacaggatattttatgttgtccttttatagccacatctacctccttcacagtctgacccccttctgtaacttctggcacccactaatcagttctctgtttctataattttgtcattcaagaatgttataaaaatgaaactatatagtatagtatgtaaccttttggggttggcttttttttttcacttagcaagattgcttggaaattcatccaagttgttgagcgtatccatagttcattcatttttattgtttattagtattccatggtatgcatgtaccatagtttgtttaatcattcacccattgaaggacatctgggttgtaccatttgttgaaaaggcaattttttccccgttgaattgcttttgcattgttgtcaaaaatcacttgggtatatttgtgtatgtctatttctggattccctattatattccatttgtctatgtatctgtccctccaccaatacagtctttttttttttttttttttttttttttttttttttttttgacagagtctcactctgttgctcaggctggagtgcagtggcacgatctcggctcactccaacctctgcctcctgggttcatgccattctcctgcctcagcctccccagtagctgagtagctgggactacaggcgcccgctaccacgcccagctaatttttttttttttttttttttttgtatttttagtagagacggggtttcaccatgttagccaggatggcctcgatctcctgacctcgtgatccacccgcctcggcctcccaaagtgctgggattacaggtgtgagccactgtgcccagctgtattccctattatattccattggtctatgtatctgtccctccaccattacagtcttcttctttttttttttttttaattttaaaattactattgttatttttgagacagggtcttgctccgttgcccaggctggagtgcagtggtgtgaatgtggctcactgagccttgacctgctgggctcaagcgatcctcccacatagctgaggccacagctgcatgccaccacacttggctaatttttaaaatttttgtagagatgaggtcttactgtgttgcccagattggtttctaactcctgggctcaagtgatgcttctgcctcagcctcccagattgttgggattataggtatgagccactgcaccccgcccttatttttttttaattactatgttgccaaggctggtgtcaaactcctgggttcctccgtcagcctcttgcgtagctgggatcacaggtgtatgcatagttttcagcatacaaattgtgtacatgttttgttagatttgcacctaagtaatttttttgaatagtttaaatgatactgtatttttaattttggtgtccacatgttgcagtatatagaaatacaattgatttttgaatgtttttgtgttctgtgaccttgttgaactcacttattatagaactgatataggactataaggagttttcttatggattctttggcattttctacatagacaattatgtcatctgcaaataagggcaattttattttttcttttccaatctgtatgccaaacttttttttttgagacggagtttcgctcttgttgcccaggttggagtgcaatggcgccatcttggctcaccgcaacctccacctcccaggttcaaacaattctcctgcctcagcctcctgagtagctgggcatgcaccaccatgcccggctaattttgtattttttttttttagtagagatggggtttctccatgttggccaggctggtctcgaactcccgacctcaggtgatccacctgcctcggccccccaaggtgctggaattacaggcgtgagccaccacgcccggcctatttttttttctttttttttttttttgagtcagagtttcactcctgttgcccaggctggagtgcagtggcttgatctcggctcactacaaccaccgcctcctgggttcaagcgattctcctgcctcagcctcctgagtagctaggattacaggtgcccgccaccacgcctggctaattttttgtatttagtagagatggggtttcaccatgttagtcaggctggtcttgaactcctgacccaggtgattcaccggcctcagcctcccaaagtgctgggattacaggcgtgggccactgcgcctggccactatactttttttctcactttattgcactagctagaacttctagcattatgttgtatatgaatggtgatagtggacattctttgccttgtttctgttcttaaggataagtcactcagtttgtcaccagtaaatacagtgttagctgtaggggtgtttggtagatgctctttatcaagttgaagaagttcccctctgttcctatttgtctgagagtttttttattatgaatcagtgttgaattttgtcagacactttctctgcatcaattggtatgaccatgtgattttttttctgtagcctgttaatatggttaattttcaaatattgagctgattaattttcaaatattgagctctccttgcatctctggaataagtaccacttggtcgtggtatatatttcttttaatatattgctgaattctgtttgatcatgttttcttaaagactttcgtgtctgttttcatgatagatactggtctatagttttgttgtaatatcttggtttgattttgatatcaggataatgctaccttaatagaatgaattggagccaagtatggtggcaaatgcctatagtcctagctactcaggaggctgaggtggtggggactgcttgagcccaggagttcaaatctagcttgggcaatgtagcaagaccccatttctaaaaaagaaaaatgaaaaaaaaaaaagaaaaataagtaaaaagttaaataaagaaaattcaatatattaaaacaaaatgaatcaggatgtgtttcctccttgtctgctttctggaagagtttgtgcctggcagtggctcatgcctgtaattccagaactttgggagtccaaggcgggtggatcacttgaggtcaggagttcaggaccagcctggccaactccttctccaaaaaaaagaaaaagaagaaaaagaaaagattggtagaacttaccgctgatgtcattcaggcctgtagatttctttttgtgggagttttaaaattacaaattcagggtcaggcgtggtggctcacacctgtaatcccagcactttgggaggctgaggtgggcggatcacctgaggtcaggagttcaagaccagcctggtcaacgtggcaaaaccccgtctctattaaaaatacaaaaattagctgggcgtagtggtgggagcctgtaatcccagctacttgggaggctgaggcaggagaattgtttgaacctgggaggcggaggttacagtgagccgagattgtgcactacattccagtctaggtgacagagcaaggctccatctcaaataaataaataaatttaaaaaaaaattacaaattcaatttccttaatatttttcttttcttttttttctttttttttctttttctttttcttttcctttcctttatcttttccttttcttttgagacagtctcaatctgtcatccaggctagagtgcagtggtgtgattatggcttattacagccttgaactgctgggctcaagccaaccttcttccttagcctcctgagtacctgggacttctgtcttaaaaaaaaattttatcctttctgcctctgtaaaataattttcttaatagaaattatatcatattggatgagttgtagtagtttgtattttttgagaggaatctgttcgtttagtttgattcatatttgtctgtgtagagttgtttatagtgtttccttatttttcttttgatgtctgcaatatctagtgatatctcctgcttcattgttgatgttagtaattgtcttctcttttttcttgtatcattcttgagatcaattttattaatcttttcaaataactagttatttgtttcaatggtttttctctgtttttgttttcagtttcattggggttttttgctttttattatttccttctgcttgcttttggtttattttgcttttttttttttttttttaataatgagatggagttttgctcttgttgcccaagctggaatgcaatggtgcattctcagctcacttcagcctctgcctcctgggttcgagcaattctcctgcttcagcctcccgagtagctgggattacaggtgcctgccaccacacccggctaatttttgtatttgtatttgtttgtgtatatgtatgtatgtgttgtttttgagacagggtcttgctctgtcacccaggctagagtgcagtggtgcagtctccgctcactgcaacctctgcctaccaggttcaagggattctcctgcctcagcctcctaagtagctgcgacttcaggcgtgcaccaccacgcctggctaattttttgtatttgtagtagagacggggtttcaccatgttggccaggctggtcttgaactcctgacgtcaggtgatctgcctgccttggcctcccaaagtgctagggttacaggcgtgagccaccatgcctggcatgtatttttagtagagacaagatttcaccatgctggccaggctggtcttgaactcctgacttcaagtgaacctcctgtctcagcctcccaaagtgctgggattacaggcatgagccactgcacccgcccgccaggcctcaaagcatttttctaagcctcattgttacagttgccacatgaatgtatctctatatatacatgtatacattttttttctttgaatcattttgaagtgagttgcagagtgtttgacacttcatccctaagtacttcaaaatacaattcataagattgaggatgattccctatagagtcttactaccatcatatcacctaagaaaaataacagtaaatcccatttccagtttacacgcaaaattcctcaattacccctttgggagctggactcatgggtttttttaaaaagtaaatccagaatccagtcaagagatacacattgtctgggcaagattgcttacctttctaatcctgggagactgaggtgagaggatcacttgaggccaagagtttgaggccagcctgggcagcatagtgggacactgtctgtacaaaagatttttaaaaactagctgagtggagtggtgtgcacctgaagtcccagctatttacttgagcccaggagttcaaggttacattgagctatgaacatgacactgtactctatcctgggccacagagcaagaaccttctctgtccccactcccctacccctagaaagatacacacatcaagtctggttgctgtctctaaatcgagaacagtcatcttatgtttagggatgtcagcatttggagagaccaggccagttgtttggcagaagatcccacattctagatttgtgtgattttctttgcatagctcccatatttcctgtaaattggaagttaggtctaaagcagtgccacccaacagaaatataacatgagccacatatgtcacttaaattttcctggtagccacatgaagaaaagcagattaaattaatttcaatcatatattttacttaatattttattgtttttttttgaggtcgagtctcactctgtcacccaggctggagtgcagtggtgcaatctcggctcactgcaacctccacctcccgggttcaagtgattctcttgcctcagcttcctcagtagctgggattacaggcacccaccaccatgcccggctaattttcatatttttagtagagatgggattttaccatgttggccaggctggtttcaaactccttacctcaagtcatccacccaccctagtctcccaagatgccaggatcataggcgtgagccacggtgcccgtcttgtttaatacatttctttattatttattctttgttaatacttattgtttacttaatatattttattgaatgtatctgactgtattcaaaatattactaatacatatcatttcagtttgcaatcaatattttaaaagtattaatgaggcattttacattcattttttatgaagttttcaaaatccagtgtatattttatatttaatggatcagctacatttccagtgctaaggagttgcacgaaactgttggtttctgtataagagaccatgggggtaagaggcttgatccaactcggggtagatgtttttgacagtaactcctggaggatagtatgtgactgaaccactggaattgctcaagtctaaacctgcttttctttttttttcaggctctgtgtgctccgggatggagcaggtgtgcagagggtgagaacccagctctgggaccaagtcacttgcttccttacttagcaagactatcgacttgagcaaacttggacctgggatgaggatgtctgtgggcctctcgctgctgctgcccctctgggggaggacctttctcctcctgctctctgtggttatggctcagtcccactggcccagtgaaccctcagaggctgtcagggactgggaaaaccagcttgaggcatccatgcactcagtgctctcagacctccacgaggctgttcccacagtggttggcattcctgatggcacggctgtcgtcgggcgctcatttcgagtgaccattccaacagatttgattgcctccagtggagatatcatcaaggtgagactggatataaagcataaatgaggaagagttctcattttccattttagttttggtggcttttccttttcaagtctaagcttcaccaattctgagctcagttcttccagtacccccttagaagagggacagccactgatgttcataagagaatccctatatactcagaatagcatcctaaactgtattcttgttcctattctgagggcatcaccatgtaatagcctttactctggaagttctactgaacactgatttagggatttgttggaaaggtctgtcacgtgtatagggaaaggttacctttaataaaacacagagttaaaccccaaaaaataggacttttatgctttgaacttactttttaaaataagatataaataaatataggagttctgctctgatttgctgatgtatgttggctaatgtaattgatcacttaagaaccaaaattagtacccaaacttcagccttgccccatatcctttggagaagccatccagagaacaatggaatctgaggcccgtgtagagaagagtgtcttggttgacccaggatagaaatcaggtgaaataggaagcttttgtgtgatgaagcaccatctctctgtctgagtttgtggaaccatggaatttcccctagatcttaaatgagtttgtttcatggacaggtgggtgggatgtacccctcaagttatgaacatcttctagagcagaagtcagcacacttcacccaagctggagtgcactggcatgatcatgtctcactgtagccttgacctcccaggctcaagtgatcctcccacctcagcctcccaagtagctgggactataagcacatatcagcacacctggctaatttttgtactttttgtagagatagggttttgtcatgttgctcaagctagtcttgaactcctgagctcaagccatcttcctgccttggcctcccaaagtgctgggattgcaggcatgcaccactgcaccaagctccaggcagtaaatattttaggctttgcaggccatatggtttctgtcagaactcctcgactctgcctgtgtagtgttaaagtagcctttacaatatataaatcaatgggcaataaaactttacggtggatactttttcttttttaattttttacacttatagtcccattctgacttgaaacagatttttgaatttcatatgtcaagaaagaaatattcttttacaatttttttttttgaggcagggtcttgctctgttgttcaggctggagtgcagtggtacagccatggctctctgcagccttaacctcccaggcttaagccttcttccctcttcagcctcccaagtagctgggactacaggtgtgcaccaccacacccagctaatttatttatttatttatttttgagacagagtttcgctcttgtcgcccaggctggagtgcaatggcacaatcttggctcactgcaacctccgcctcctgggttcaagtgattctcctgcctcagcctcctcagtagctaggattacaggcgcacgtcaccacgcccagctaatttttaaattttaatttattttaattaattaattaattttgagatgaagtctctctctgtcacccaagctggaatgcaatgacacaatctcagctcactgcaacctccatctcccagattcaagtgattctcctgcctcagcctcccaagtagctgggattacaggtgcgcaccaccatgcccggctaatttttgtatttttagtactgacggggtttcaccatgttggccaggctggtctcaaactccttatctcgggtgatctgtctgcctcagcctcgcaacacccagctaattaaaaaaaaatttttttttagtttgggcgtgatgacttacacctgtaatcctagcactttgggaggctgagggaggcagattgcttgagcccaggagtttgagaccagcctgggcaacatggtgaaaccccaactctacaaaaaatacaaaaattagttacaaaaaattttttttagatagggtcttactctgtctgccaggctggtctcaaacttctggttcctgccctacagcattccttctctagaggtgtggcctacatgtgaactaaaagtattctgagacctatactttgttaggtattttctgtatgatctctgcattcagacttctacatacatttagtgctctggctttctgttggtgtgtttaaaccaccaaaacagtggtttaactcaaccagcttgttttactcaacattttttggccaggaattcagagataaggaaccttatctctgtttcacatggtactaggtagagtggctcacctggggctggggaattcacttctaagttggctcattcatggctggcaagttgatgctgcctgttgacagggagcttagctggggctgttggtcagtgtccttgattcctcttcatgtagacctctccatgggctgcttaggtgtcctcaaagcacgatagatggattttaagattaggtatttcaagaaataggaaaggaagctgcccaatctcttaaggcccagaacttgcccaacaacacttccattttactccattggtcaaagcacatatagactctgatgggtgtttaccagcttgcccccgtgacccgtaagccccaggcttctagattagaaatggccacaagttctatgccactctttccttgcagaggtgagtctgtgcccttttccctgaatctgggtggacctccccttcagcctgctcccttcttgtgttcgcctccttttccctgttggtcaccttctttttaagccgtggcttgttctcagagtgaccttgtggtctctggggcaaccgtatttctgaagatcttggcccatgtagcagctcacatgtagcagttcacagagctcttcctagtttgcagagtgtttgcataaagtttccttgtcatctttgtaatatcccagcaagctagatgctaatctgaccattagatacagagtggtggctctggaaggtacatttcttgctcagcttcagatttaatcctgaatatccataccctgcattggttgcctttgttccagtagccattggaatgttttacgtggattaatcttggccagaagtgccttgaactgatacagcaaactagatatttgttgtatttggcagagagatgtttaacaggcttatttaccaagggggtgaagagattttactgagaaaacaggattttatctgagcatatttatctgtggaaagcagaccatttagtagctgatgagcttgtagaaaaacagatgaagatgtgtgactcttcgttccagtcttaaccaccgggattatatgggttggggagtcagaaggtcagaaagcctgatcttgttcctagagctttggtttctgcatctgtaaagcaggatttagaaagtctttatattaaaggaatttttttcttattataataatgatgcataaatgtacattgtggaaaatatagaaagggaaaaaaaatctgtggtttctaccattcagagtgaagtcctctaccgttttctatgctcatcaagagatggtcatgccacatacacacacagacgtcttaacatattagacagacctaggatcatagtggatatattttttaaccttttttttttgagacagagtctcactttgttgcccaggctggagtgcagtggcacagtcccggctcactgcaacctccacctcccgggttcaaatgattctcctgcctccatctcccgagtagctgggactacaggcatgcgctaccacacctggctaatttttgtgtttttttggggggatggagtttcgctcttgtcgcccaggatggagtacaatggcgtgatcttggcctactgcaacctccgcctcccgggttcaagcgattctcctgcctcagcctcctgagtagctgggactataggcttgaaccaccactcctggcatatgtgtgtgtgtgtgtgtgtgtgtgtgtgtgtacacatacatagacacacacacacatatatatatattttttgagatggaatcttgctctttcgtccaggctggaatgcagtggcgtgatctcagctcagtgcaacctccgcctcctgggttcaggctattcttctgcctcagcctcctgaatagctgggactacaggcatgcgccaccacacccggctagggtttcaccatattggccagactggtctcaaactcctgacctcgtgatccgcccaccttggcctcccaaagtgctggggttacaggggtgagccaccttgcccagcccacgttttgtatttttagtagagatggggtttcaccatgttggccaggctggtatcaaactcctgacctcaggtgatctgcctgcctcggcctcccaacgtgctgggattacaggaatgagccactgtcccgggcctattttttttttttttttaacatttttcattcactgtagcttgagtatttttttacctgtagtcattaaatgttatgtgggaatgtaacttttaatggctgcctaatattcttgtgttgttttttttttttttttgagacagggtcttgctctgttgcccaggctggagtgcagtggcactatcatggctcactgcagcctccacctcctagcttaagcaatcctctcacctcagccttccaagtaactgggactataggtgtgggccaccacacccaggtaatttttaaatttttttgtagagatgggtctcactttgttgcccaggccggtcttgaactcctaggctcaagtgatcctactgcctcagcctcccaaagtgctaggattacagacatgagcatcacgcccagcctgcccactattcttgagtgtgtgaggaacatttcagatattggtcctaaggatgcttgttgaggcgaccccagtcatcttgggcccactggtactgcccttcatccaagctactgcttgcttacttcttcctaggctccccctgcctttgttcttgacctctcaatttgttctccatattgagtcaagagatgtctttatctttctaaagcttaaatctattcaaaatccctttttactgagctgcaccaagaatcgaaccccatatgatctggcctgtgacctcctctcccacatctttttctttgtatactttgtcccagccacactggtccttttctgcccagtgaacacacctagcttagggtccaccccaagcttttgattctttcttcttttgtcatgggtacagccatatctttcaggctcaacttggacatcatctgttggagccctttccttccaccctggtaaaagcagctttcccacaaaactctgtgtcatcttcttcttagcagatttctgtatttgaaattcttgttttttgttagtcatgtttgccttccatcacatagtaggccctcaaatgtttgtaggctggcaaagggaatgaggactcatttagcacctgcaaacctgttttggattgtcaaagcctgccttgctgtatgcagggagaaatgctagcctgtgctggggcagcaagagactaagttcacctgctgccccacagtgtactatggtcttgccgtgttttgaggcctcaggccctgtgacacacacactttcaaatgacttcttgttgtattttcacattggaaagtggccccaccctgtccctctaacacttgactgtgtgtttattgatccattattagtacaaactcactgtttcctaatattcaatagtttattgctcatcttaattattgtggtgttcaaattgtcccagatttgaccattgggagcccctttaagttggttcctgtattcttgtgatatgcccctgtcaatcattaagtgcttccttactttctgggataagatgctccaggcctgtcttgtattcaccctccctcagccatttcttcaaggagcctgattcctttttgtggtgaatgttattgggggttcagatctgggtgctaggtgagttcattgccactgggatttccttgcttctttgagccgttcagcagatagtgtggctccatttttagatgacaaagtggtagctaagtgacttatctcggagcacatagcttgtagggacagagttagtttgtagggagcacccaagaggcttttttttttttttttttttaattgagacggagtctcgctctgtcgcccaggctggagtgcagtggtgctatctcggctcactgcaacctccgtctcccaggttcatgccattctcctgcctcagcctcctgagtagctgggactacaggtgcctgctaccatgcccggctaattttttgtatttttagtagagacggggtttcactgtgttagccaggatggtctcgatctcctgaccttgtggtccgcccgcctcggcctcccaaagtgctgggattacaggagagagccaccgcacccagcccccaagaggcttcttgagcctgtagtatataaggtcttatcttcaggtaactgacctgtgtttattttgtgtgtcagcatgctttggaattataatttaatgctttgttctagtcagttttagaagacttccctcgtagttgtctgttaccgtgcctctgacctctgacactgcagggctgtggaagagagagacacactgggtatgccttgacatggatggtgtcacatgtgatgagacgggtttgtacaacctgccatgcatgtgggcatctttgagccagagcagaagctgacctatcattgggcccaaaactgattgctaagtaaatggctggtttacagttctggcttttggaatgaaggctgcaggaccacaggtttggaagatcagggccaaccttgatgaaagtgaaaggagagtctgagtgaaaggtgatgtctggctggtaagctatcattgttccctgtgtagggttctagatacttggcataggacctgtttcgaagtagatgcctaattaaaggcttgctatgtgataaagcattaacagaggtggagcaacaacggtaagacatgtgggacagtggtgggttgcagagctccatgtggggaatttgagaagttgttgggagcacatggagttgggctcctcagcagagaaaggccagtgcccacatgtggcagggttgtcaatttgagtgaaagtaaatggagagagaagtgggtccgtaaggggcttttcaaaggctttcttccccaagtatcagaggaagaagaagctggtgaagaactgaggtaggaggtactgagcctggcctgcttcctcctaagccccatgtgggatgtccagatgagggttggtctatttgggcagggctgagttgccaaatgaagactcaggtggaacgtgatctgcatggtctgtcccaagacctggtgagattagatggcatagttgtcatacttgctgattggttaggggatatgcatgggggttgacggtagaataatccagtgtttatagcacacttagatgctggtctgttgaagggctagaaaattaagttctcattctaagtgagctgtgttgactgcctgaaaagcctggcctgacctgtgtgggcttctggttactttatttcaggaggttgtgctgcctggtgtcagtctgttgatatacccaacaaagcgcctgaagcaaatgcttcgctacattttatattggtgtatactttacacgtcagtctgcagggtcaggagaataaagaagggacctgcctgcgcagagggtatagccccaagatcctggctcttctgctctgccccacacctgacactccttcatgttctgacagctcttgtccagtctcagcctggccagcacttgttgcttaaccaatgctgttctccagttctgccttgagaattgatattctgttattgaccccagcttaactcacacatggactgctttgaccagacgtgtgagaaggggctatgggctccctcaaggtcctcatggccagcatagcttttttgagagacctgtgccatggggcccctgtctcctctcttgtggccttaggaaaccctaaagcagatagtcttgatgtttgctcccttgaatcagactggatgcctgtatacaaagctcagcccagctcttgactgtccttggctctcacagctacttttggagctgagaggaccctttctccttatcagggtctgtggggatgggtcccacagaagccaacctggccttcactcagtggatcctggcctgtcctgggctggcaacagtatgagccacccttgtctcccttgggtttggtgctggatccctgcaggcacctctcctctgacttgagtgtgggactggctgagaaatgtgattatatgccaggagatgttctaacctctcctcgaaggtggtcagggagagacatctttacatttcatgaaatgcccaggctgcccctcagatgtctcattttggtagagcctgcaaggatggcttttcatccctgcttccctttagccttcttgcctgcagacaatgtggaccttccctcagcccaagatgttggcaggctgggagcagcccttcctgggactgtgctctcagctgctggcacgggcccccaattgttcccctgagaacaatgggcactctctttggctaggaaggggagttcatgaaaggagcatttctttggatgcttgagagctttgtggcagccccgcctgtgtgagatgagctgcagtttcttcctgccttgagtgggtgttccgcatttctgaatggaccccttgtgaacaaagcacaccgggaaatgcgggttggcgatggaaggcatccttgggggtgggctgtcagaatggcatttgcccagattcatcaactcaaatacttagtgaaaatataggcttctatgtttaaaaaaatcactgacaaagactgaaagagaacatgacaaagtaaaatagatgatgtcagggctccagattgaaggactgattctgaggattttattagtggctctaactatgttaagctgccctttacttttttggggaaaaagactaatttttgcttttagatgagaacagtgaacatgagataaaatgctttgtagctcagaccagaagggcattgactgggctgaacagtgtgttcagtattgatcctgtggcgttggtgtttgtggcaggggccaggttctgctttgaggtttgcatgtctaacagcaggaccaatgatgatcccttgtgggtgtgactctgctaaggccgcatgtacttaacttccctcatgattttttacctcaggtggggacaaaggcctggccctctgaccttaagctagaggctcttcctgttggtctgtgccatctccctgcagcacagccatgtgactgcccatgttcctcattagcagaaggccacagagcagatttatctcattgtcttagtccatggcacagacacaggataaccctgagtgtgagtcagcaccaaggctgatactccatcagagggtccatctcctgcttccctgagcagaggacacacagtcagcttgcaatgaggaggccttgactgcctcatgtgggagtgcctccagggtgttgtctatggtacccagccatagatggctgcaggttggcccagctctgcagcctgactagggatgagggagctgaaaggtcgggaattgatgaccaggtctgaaggaaagcagggactttgccaacgcaggcctctgcaggtccagcattgggatgtcacggggcagcagtctgtatcactttgaaatggatctgttgggggacacggggaagggcccagcccacaccttaagggctgacatgtccagaggtattgatggaaggtgccaccaccaaggcccaaagttccctccaaaagatttgtagtgagaaggaatgctcaccaaatcagtttctcgattatgttattcttaggatccctctttactggccatcatcagctgtgaaagtgctcgtacatctgtctagagtctttgcttgccatttctaagtcctgcaactcctgcatggataagacatcatcttgtgggccctgaatgatacagcttgtgttttcttggtttcataagccaattataattatttggccctagagagagcctaattcttcagttttgtttttcagctatgcataactgactcagactatgggtggaaacctgcttttatgagaactaagtaatagaagctcctccagccatttgctctttcttcaggtgtggaatggtactggcttggggtgagagactttgcttgagtttcatggtgtttttttttgttttgtttttttgtttgtttttgttgttgttgttttttgagacagagtctcgctctttgcccaggctggagtgcagtggcgcgatctcggctcactgcaagctccgcctcctgggttcacgccattttcctgcctcagcctcccaagtagctgggactacaggcacccgccaccacacctggcttattttttttgtatttttagtagagacggggtttcaccgtgttagccaggatggtctcgatctcctgacctcgtgatccacccgcctcagcctcccaaagtgctgggattacaggcgtgagccactgcgcccggccgtctttgtttttttgagacaggtcttgctctgtcacccaggccaaggtgcggtggcgcaatcatagctcactgcagccttgacttccccaggctcaagcaggcttcccacctcagcctcctgagtagctgggaccaccacgcccagctaatttttcttttttgtaaagacgacgttttgccgtgttgcccaggctgatggtgtttagtgcctacaggtgctgtatctttatcaaagtcagtgtttgatggtctattgtgtgcagagggcatcagtaaagcagctggagggagtgtcccagagactgaggagtgaccaaaatgttcaaatttggcagttccctggtcattggtcatggtgatttattgtgggcatcacagttttagacctttgtttttttttttgagatggagtctcgctctgtcgcccaggctggagtgcggtggcacgatcttggctcactgcaatctccacctcccgggttcaagcaattctcctgcttcagcctcctaagtagctgggattacaggtgcgcaccaacatgcccggctaattttctttttactttattttatttttttttgagacaaagtctcactcttgtcccccaggctggagtgcaatggtgtgatctcggctcactgcaacttccgcctcccgggttcaagcaattctcctgcctcagcctcccaagtagctgggattacaggtgcctgccaccacacccagctaatttttttgtatttttagtagagatggggtttcaccatgttggccaggctggtcttgaactcctgacctcaggtgatccgctcacctcagcctcccaaactgctgggattacaggcgtgagccactgcgcctggcttttttttgtatttttagtagagatgggctttcaccatgttggccaggctgctcttgaactcctgacctcgtgatccacccgcttcggctcccgaagtgctgcgattgcaggcttgagccaccacgcccgcccccctcccaccccttttttttttttttaagactgcatcatggtatgttgcccaggctgggctcaactcctggcctcaagtgatcctcccgcctcagcctcctgagtagctggggttataggtgtgagccaccatgcccagctcagtctttggaactttttgtggatctgcccaggttttgtggttttgattcttaatgttttgggatgcactttgtgggtcaggatttcagaagctggttataggcttttcaagatctttttttccttaatgctagacttactttctatctcagaagcacatcgtctctaagggttgccgaaaccagaaagttttctggcctttttggtggcctgtgagctacagcctgtgatctccttaaatcaggctttcttgggcagtttctgcctcctgcagatgtgcctagtaactgtctgaaggcttctttcaagacagcaactgtaaggtcagcaggccttgcctctcccagctccaaggcagtgttggtgtttggtgttggctggtagagagacctgaaatgctggaggagttccacggtgggaccagcttgtcaccccaaactcatgtgcctctgtgcctctgctgctaaggacctcagagccccaattcctgcctcttttctgcccttcctggcctggcagcccttagaggattagttgtgggtggctgtggtatagtaggcagaaatctttataccaggcaaaccaaggactctaaggatagtcaaaatcccatgtgtaaatgtgccaggatctagtgcacccattttattaattatagagacaaagtctcactatgttgcccaggctggtcttgaacttctggcctcaaacaatcctctcgccttggcctcctagagtgctgggattacaggtgtgagccactgtgcctggcctagtccacctgttttaggttgaagaggtgaaagcaggtctttgggttctcacctgcagcaggtcccccacttcccccatgccccttagtgttttcatctacttctggtagacaagtgttgacaggcaactctctgagcttccccagcctcagctttgtttctctatatgattttggctccctcattgcctgttgcctccccacttaccttagatgctttgagcttctgttttgtggtatgaacagggagacaggaccccactgtgcactgggtccaaggtcagttgggaaggagaactagggagaggagcctggacatgccctgggcccctttgtgcagtagcctcaaattacttactggttcttctcagagcccatgggagccataggaaccattattggcagagtccatcatgtgcctggcactataatcatatgaggttgggattgtttccctgattttacagatgacactggggccaaggttgggtcaagcgccagccagcctggagctgggattcgcagtgaaagatgggccttctcatggagtagaagaatgaggattgttatccccattgttatggggatgagtcctggcttgtcattgggctgcatctgtgagaaggaactttcatatcctgtttcacccttaggatctgagaagaagagaagtgatccagagcctataagttttgaaagctgaggaggtatccagagttcccgtggactgagggcctcctgctttgttagggtagagcaggtgctttatcccctagcacctccgggatgagctaagccctgagctgagtccctttgggagggtggggctcctagtggaatcctttggggactgtggacactgccaaaccttcaggcatgttcagactttccaggtaggggctttccccaaatggccttgaaaatgcagaagtgccagtgtgcctgtgacttggccaggttccttgtggaaattaggtttgttttgcttcctgtagcccttctattaaaatgaatcatggctgggcacggtggctcacacctgtaatcccaacactttggcaggctgaggcaggaggatcactgaagcctaggagttcaagaccaatctggacaacataggaagacccatctctacaaaaaagtaaatattggccaggcctggttgtacacacctggctcaagtagtcccagctattcaagaggctgaggtgggatgatcacttcagcccaggagtttgaggttgcagtgagctgtgattgcaccactgcactccagtctggtgacagtgagtgagaccctggctcaaaaaaatgtatatttaagggctgggcatggtggcccacacctgtaatcccaggattttgggaggctgaggcaggaggattgcttgagcccagaagtttaaagccagtctgggcaatataatgagaccctgtctctacacacccacacacacaaaatacccgggcacgtgcctatgtcccagctacttgggaggctgaggtgggaggattgcttgagcctgggaggttgaggctgcaatgtgccatgatcacactactgaattccagcctaggcaacagagtgagacccttatatatacatatacatatatgtatataaagaatcattgccactcaaagtgctgcttgggcctgatgtcaccagtaccactcaaagtgctgcttgggcctgatgtcaccagtaccactcaaagtgctgcttgggcctgatgtcaccagtaccaagactggagatgcaggggttttagaacattcaggttagtactttttcttcctgagtagtacccatgatcctggagtgactgcctgcttcagggctgtagatctagacacctgtcctgcagccaggtagtggccgggacttcaggaccctggggagccaaaatttgcagaggggaaagcaggtgactaggttggagagacttggctcttatattagtctgttcttgtggtgctataaagaaatacctgagactggtaatttatagagaaaagagttttaattggctcatggttctgcaggctatacagggaatatagtggcttctgcttctggggatgcctcaggaagcttccaatcatagtggaaagcaaagggagagctggcgcttcacacggctggccggaacaggaggaaggctgggggaggtgctacacacttttttttttttttttgagacggaatctcgctctgtcgcccaggctggagtgccgtggcgtgatctcggctcactgcaggctccgccccccgggttcactgccattctcctgcctcagcctcccgagtagctgggactacaggcgcccgccacctcgccccgctaattttttttatttttagtagagacggggtttcaccatgttagccaggatggtcttgatctcctgacctcgtgatccgcctgcctcagcctcccaaagtgctgggattacaggcgtgagccaccgcgctcggcctttttttttttgagatggagtttcgctcttctgcccaggctggagtgaagtggtgcaatctcggctcacttcaacctccgccctctgggttcaagcgactctcctgcctcagcctctcgagtagctgggattataggcgcctgccagcatgcccagctaattttcgtatttttagtagagacagggtttcaccatgttgaccaggctggtcttgaactcctgaactcaagtgatccacccacctcagcctcccaaagtactaggattataggcgtgagcctctgctcccggcctacacacttttaaacaaccagatcttgtgagaactctgtcatgagaacagcactaggggggtggtactaaacgatccatgagaaatcacccccatgatccagtcccctcccccagaccccacctccaacactagggataataatttgacatgagatttgggcagtgacacagatgtgaaccatataagctctgtttttctgacatgacttgccccaaagacatgggtaaactttacatggctgcaaagccatgggtagacaatagtaaaagaatttggtcacgtgcagtggctcacaactgtaatcccagcactttgggaggctgaggcagccagatcactggaggccaggagtttgagaccagcctgggcaacatggcgaaatcccatctctaaaaaaagaaaaagaaaaaaaaaattagccgggcatagtggcagtcacctgtggtcccagctactcaggaggctgaagctggagaaacatttgaacccaggaagcagaggttacagtgaactgagatcgtgccactgcactccagcccaggtgacagagtgagactctgtctcaaaaaataaataaataaataaaattaaataaattgtgaaagaatttgttctcagtagtcaaaataaagctcatttctcatctcccatctcccaatttgaaattgagcattataacttgctttggtgaaacctgacgttttctttagtgtttattgtttgttacataatattaacatgtttttaagttacttggcatagttaaaataagtgtatggctttcaaggatggcttcgctgtgggagccttcctatagaatggcctgagtttgttctgtaggaactgcctgatagcagaactttggctgttggggtcagcagaataggaagtgagccaggctgggaagccttgagaaggcagggcccttctctattttctgttaaaaagtgcccttgtggccgggtgcggtggctcatgcctgtaatcccagcactttgggaggccgaggtgggcagatcacgaggtcgggagattgagaccatcctggctaacacagtgaaaccccgtctctactaaaaatacgaaaaattgccgggcgcggtggctcacgcctgtaatcccagcactttgggaggccaaggcgggcggatcacgagctcaggagatcgagaccatcttggctaacacggtgaaaccccgtctctactaaaaatacaaaaaattagccgggcacggtggcgggcgcctgtagtcccagctacaggagaatgaggcaggagaatggcatgaacccgggaggcggagcttgcagtgagccgagatagcgccactgcagtccggcctgggcgaaagagcgagactctgtctcaaaaaaaaaaaaaaccaacaaaacacgaaaaattagccaggcgtggtggcgggcgcctgtagtcccagctacttgggaggctgaggcaggagaatggcgtgatcccgggaggcggagcttgcagtgagccgagatcacgcccctgcactccagcctgggtgatagagtgagacgctgtctaaaaaaaaaaagtgcccttgtatagttgaagacagacttggagaggcaacaggttgctgacttatcaacactgatggagcgcctagccactcaggaatgtcccatggttctagccagccatttggctcccttcttctagtccctctcaaccttgttcagtctcctctgcagttggcttctccttagttggctttacccagacctctattccaggccttccctgtcacctttcttcatcctccctctgccatgtgagctcgtgtcctactggtggccattgggatggagtagagttaggaaataaccctcaggtgaccaaaaaccggggtccacaccctaattcacctactctccacctctcaggacccctacttctatcccactcctgtaaaacatctcatgggccttacttatttgcttcatggtggttttttgtttgtttgtttttgcaaaacaagagcattcaacctttattttatgtgtttatatacggatgtggcagttttatttaatataaatgaactgaaaaatgtaaagcttagatttttcagccaaaacatttgaaatagtgtgatttttttttttttttttttttttttttgggacagagtctcactctgtcattcaggctggagtgcagtggtgtggtctccgctcactgcagcctctgtctcctgggttcaagccattcttgtgcatcagcctcccgactagctgggactacaggtgtgcaccaccatgcctggctaatttttgtgtttttagtagagacgggattttgccatgttggccaggctggtctcaaactcctgacttcaagtgatccacccgcctcaacctcccaaagtgctgggattacaggcatgagccaccacgcccagccaataagtgtgatttttatggttaaaaaatatttttaaagaaaaatatatttagcatttttcatgaatttgaagacacccatgtttacagatgttttaacatttctgatgtttgatggtgagtgacagggtgggagattcttaattagatgatggcacatcttagagttgatgcaatgtggtatcacaaaagaaacactatttgctaacccttaatgagtggctcagagagcaattaggaaatgataaccctgataataataacgccttttttttttttttttctgagatggagtctcactctgtcacccaggctgggttgcagtggcgtgatctcagttcactgcaacctccgcttcccaggttcaagcaattcttatgccttagcctcctgagtagctgggactacaggtgcctgccaccacatccgactaatttttgtacttttagtagagatggggtttcgccgtgttggccaggctggtctcgaactcctgacctcaggtgatctgcccaccttggcctcccaaagtgctgggattacaggggtgagccaccatgcctggcccaataataataattcctaacaatgtcagtaatctcatggttaaaaaaaattttttttttgaaatagagatgggggtctcactgtgttggccagggtggtctctaatgcctgctggcctcaagcaatcctcccgccttggcctcccaaagtactgggattataggtatgagctaccatgcccagctttctcatacatttttgctgggtgtaggaaacctgtttaaattagtttaaaggggggaaattatctagtttctgataaggctgctgcctggacacctgggtacaagcctggggtctcctttccaaatcactcaagtactttgttttttggcaggaggaagcccagggaaccagaggcactggggaatccagcagtcagtggctttagctttcctgccacagggagggggaagcattttagggtccccctattgataagcagtggtcatgaagggcagtcctaagctgagtctgggagtgtgatgtactgtgttttggccaatttgtctctggtacttgttataggacaatggagatgagagtgccttcaggccaaccagagaatggggggtgtcacttttcttgcattggggcaggagtaggtgtttcccaaaagtgaaaggagccacagatggggctggggcaagagctaagtcatattttcctcttgtcagctgttggtggaggtttctatggtggcatttccttgggtgctctgagtatgaccagttatatctctgtggaaagggacttccacaacaaaagaagtctgggagatgctgttgagttggaataaggtgatgagctccttaggcctgcttctgtgcctggtctctccaagggcagaagaggtctctgtgtatttcctgtacttgattgactgtagaacctctctcttttagagagaccagagctccttgggattcctttagtaagcactattctagaatagttgattctagtctctgctccctgggtaccacagtttgtcttgagatttgagaagcagggtaaatgaaaacattagatacaactaactctctttgctacacaaatgtgccccactacacttgaatgtaacgtgatgttgtaatgtgagtccttcagagcgttgagccttcccttgtacccagacctgtgtgcatctccactttggggcaggaatggtcctccaggccaggcatgtgacctgagcaaagatgtggtgtctgcagagggccggggagaagtctgaggcaagggtggaaaggaatgctgagggctggaatgtgacttgattcttcaggtagaatgggcaagtaggtggcagggcgtgtcactgttataaaatccacaacagtcgtgagctccatggcatcagttcttgggaatgagacatgtgcttgagtgccagcactgctgtctccccactgtgtggccttgggtgagatatttgcctcccaagttagcccatctcatagccctagtcttgtgacaggtaaataaactcagttgtgtctctctaggggggaggaatcagcttccccagcaggcattctgaattataccttaaacctgtcacacaattcaggttaactgttgtgatcatattcaggttatgcagtgacaatagtatttttaatttatgcttgtgtctcttctaggtatcagcggcagggaaggaggctttgccatcttggctgcactgggactcacagagccacaccctggagggcctcccccttgacactgataagggtgtgcattacatttcagtgagcgctacacggctgggggccaacgggagccacatcccccagacctccagtgtgttctccatcgaggtctaccctgaagaccacagtgagctgcagtcggtgaggacagcctccccagaccctggtgaggtggtatcatctgcctgtgctgcggatgaacctgtgactgttttgacggtgattttggatgccgacctcaccaagatgaccccaaagcaaaggattgacctcctgcacaggatgcggagcttctcagaagtagagcttcacaacatgaaattagtgccggtggtgaataacagactatttgacatgtcggccttcatggctggcccgggaaatgcaaaaaaggtggtggagaatggggcccttctctcctggaagctgggctgctccctgaaccagaacagtgtgcctgacattcatggtgtagaggcccctgccagggagggcgcaatgtctgctcagcttggctaccctgtggtgggttggcacatcgccaataagaagccccctcttcccaaacgcgtccggaggcagatccatgctacacccacacctgtcactgccattgggcccccaaccacggctatccaggagcccccatccaggatcgtgccaacccccacatctccagccattgctcctccaacagagaccatggctcctccagtcagggatcctgttcctgggaaacccacggtcaccatccggactcgaggcgccattattcaaaccccaaccctaggccccatccagcctactcgggtgtcagaagctggcaccacagttcctggccagattcgcccaacgatgaccattcctggctatgtggagcctactgcagttgctacccctcccacaaccaccaccaagaagccacgagtatccacaccaaaaccagcaacgccttcaactgactccaccaccaccacgactcgcaggccaaccaagaaaccacggacaccccggccagtgccccgggtcaccaccaaagtttccatcaccagattggaaactgcctcaccgcctactcgtattcgcaccaccaccagtggagtgccccgtggcggagaacccaaccagcgcccagagctcaagaaccatattgacagggtagatgcctgggttggcacctactttgaggtgaagatcccgtcagacactttctatgaccatgaggacaccaccactgacaagctgaagctgaccctgaaactgcgggagcagcagctggtgggcgagaagtcctgggtacagttcaacagcaacagccagctcatgtatggccttcccgacagcagccacgtgggcaaacacgagtatttcatgcatgccacagacaaggggggcctgtcggctgtggatgccttcgagatccacgtccacaggcgcccccaaggggatagggctcctgcaaggttcaaggccaagtttgtgggtgacccggcactggtgttgaatgacatccacaagaagattgccttggtaaagaaactggccttcgcctttggagaccgaaactgtagcaccatcaccctgcagaatatcacccggggctccatcgtggtggaatggaccaacaacacactgcccttggagccctgccccaaggagcagatcgctgggctgagccgccggatcgctgaggatgatggaaaacctcggcctgccttctccaacgccctagagcctgactttaaggccacaagcatcactgtgacgggctctggcagttgtcggcacctacagtttatccctgtggtaccacccaggagagtgccctcagaggcgccgcccacagaagtgcctgacagggaccctgagaagagcagtgaggatgatgtctacctgcacacagtcattccggccgtggtggtcgcagccatcctgctcattgctggcatcattgccatgatctgctaccgcaagaagcggaagggcaagcttacccttgaggaccaggccaccttcatcaagaagggggtgcctatcatctttgcagacgaactggacgactccaagcccccaccctcctccagcatgccactcattctgcaggaggagaaggctcccctaccccctcctgagtaccccaaccagagtgtgcccgagaccactcctctgaaccaggacaccatgggagagtacacgcccctgcgggatgaggatcccaatgcgcctccctaccagcccccaccgcccttcacagcacccatggagggcaagggctcccgtcccaagaacatgaccccataccggtcacctcctccctatgtcccaccttaacccgcaagcgcctgggtggaggcagggtagggcaggggcctggagacgacatggtgttgtctgtggagaccggtggcctgcagaccattgcccaccgggagccgacacctgacctagcacacactgacacaggggcctggacaagcccgccctctctggtcctcccaaaccccaaagcagctggagagactttggggacttttttatttttattttttgcctaacagcttttggtttgttcatagagaattcttcgcttcatttttgatggctggctctgaaagcaccatgtggagtggaggtggagggagcgaggaaccatgaatgaactcgcaggcagtgccgggcggccccctggctctctgcgttttgcctttaacactaactgtactgttttttctattcacgtgtgtctagctgcaggatgtaacatggaaaacagtaactaaagattaaattcaaaggactttcagaagttaaggttaagtttttacgtttaatctgctgtttacctaaacttgtatgtataatttttgggtgggtatggggaattgctttgctaaaaataagctcccagggtgtttcaaacttagagaagaccaagggacagtattttttatcaaaggaatactattttttcacactacgtcaacttggttgctctgataccccagagcctgattgggggcctcccggccctggctcacgccaagtccctggtgctgggtttgctctcccgctgttgccaggggctggaagctggaggggtctcttgggccatggacatccccacttccagcccatgtacactagtggcccacgaccaaggggtcttcatttccatgaaaaagggactccaagaggcagtggtggctgtggcccccaactttggtgctccagggtgggccagctgcttgtgggggcacctgggaggtcaaaggtctccaccacatcaacctattttgttttaccctttttctgtgcattgtttttttttttcctcctaaaaggaatatcacggttttttgaaacactcagtgggggacattttggtgaagatgcaatatttttatgtcatgtgatgctctttcctcacttgaccttggccgctttgtcctaacagtccacagtcctgccccgacccaccccatcccttttctctggcactccagtcccaggccttgggcctgaactactggaaaaggtctggcggctggggaggagtgccagcaatagttcataataaaaatctgttagctctcaaagctaattttttactaaagtttttatacagcctcaaattgttttattaaaaaaaagatttaaaatggtgatgcttacagcagtttgtacgagctcttaagtgttgattccatggaactgacggctttgcttgttttgattcttttccccctacttttcctaatggtttaaattctggaattacactggggttcttttgccttttttagcagaacatccgtccgtccatctgcatctctgtcccatgactcaggggcgcccactctgcttcgattctcctcctgtggaagaaaccattttgagcatgacttttcttgatgtctgaagcgttattttgggtactttttagggaggaatgcctttcgcaataatgtatccattccctgattgagggtgggtgggtggacccaggctccctttgcacacagagcagctacttctaagccatatcgactgttttgcagaggatttgtgtgtgctgcctcaggaggggagggctggtaggagggggggagaggtctctgtcctactgctctccagagggcatttccccttgcgccttctcccacagggcccagcccctctcccctgccccagtccccagggggtactctggagtgagcagtgcccctgtgggggagcctgtaaatgcgggctcagtggaccactggtgactgggctcatgcctccaagtcagagtttccctggtgccccagagacaggagcacaagtgggatctgacctggtgagattatttctgatgacctcatcaaaaaataaacaattcccaatgttccaggtgagggctttgaaaggccttccaaacagctccgtcgcccctagcaactccaccattgggcactgccatgcagagacgtggctggcccagaatggcctgttgccatagcaactggaggcgatggggcagtgaacagaataacaacagcaacaatgcctttgcaggcagcctgctcccctgagcgctgggctggtgatggccgttggactctgtgagatggagagccaatctcacattcaagtgttcaccaaccactgatgtgtttttatttccttctatatgattttaagatgtgttttctgcattctgtaaagaaacatatcaaactaaataaaagcagtgtctttattacaacgc

SEQ ID NO: 8 - DNA, 3397 bp, Homo sapiens
aggggccgcggagccgcggctaaggaacgcgggccgcccacccgctcccggtgcagcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttggtgagtagcgggctgctgggctggccggggctttcgtggccgccgggccgctcggtgggacggaggcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctctggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtcgagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcaaatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaat

SEQ ID NO: 9 - protein, 489 aa, Homo sapiens
MKELDPKNDISEDKLSVVGEATGGPTRNGARGPGSEGVWEPGSWPERPRGDAGAEWEPLGIPQGNKLLGGSVPACHELKAFANQGCVLVPPRLDDPTEKGACPPVRRGKNFSSTSDLSKPPMPCEEKKTYDCSECGKAFSRSSSLIKHQRIHTGEKPFECDTCGKHFIERSSLTIHQRVHTGEKPYACGDCGKAFSQRMNLTVHQRTHTGEKPYVCDVCGKAFRKTSSLTQHERIHTGEKPYACGDCGKAFSQNMHLIVHQRTHTGEKPYVCPECGRAFSQNMHLTEHQRTHTGEKPYACKECGKAFNKSSSLTLHQRNHTGEKPYVCGECGKAFSQSSYLIQHQRFHIGVKPFECSECGKAFSKNSSLTQHQRIHTGEKPYECYICKKHFTGRSSLIVHQIVHTGEKPYVCGECGKAFSQSAYLIEHQRIHTGEKPYRCGQCGKSFIKNSSLTVHQRIHTGEKPYRCGECGKTFSRNTNLTRHLRIHT

SEQ ID NO: 10 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

(19) United States
(12) Patent Application Publication
(10) Pub. No.: US 2023/0106465 A1
(43) Pub. Date: Apr. 6, 2023

(54) 7-Gene Prognostic and Predictive Assay for Non-Small Cell Lung Cancer in Formalin Fixed and Paraffin Embedded Samples

(71) Applicant: West Virginia University, Morgantown, WV (US)

(72) Inventor: Nancy Lan Guo, Morgantown, WV (US)

(21) Appl. No.: 17/906,315

(22) PCT Filed: Mar. 19, 2020
(86) PCT No.: PCT/US2020/023597

§ 371 Date: Sep. 14, 2022

Related U.S. Application Data
(63) Continuation of application No. PCT/US2019/036953, filed on Jun. 13, 2019.

(51) Int. Cl. G01N 33/574; C12Q 1/6886; A61K 33/243; A61K 31/282; A61P 35/00

(52) U.S. Cl. CPC G01N 33/57423; C12Q 1/6886; A61K 33/243; A61K 31/282; A61P 35/00; C12Q 2600/158

(57) ABSTRACT

A method of providing a treatment to a patient having non-small cell lung cancer is provided comprising extracting total RNA from a formalin fixed and paraffin embedded tumor of non-small cell lung cancer of a patient after the surgical resection, generating complementary DNA (cDNA) of the extracted total RNA from the patient's tumor, quantifying of mRNA expression of 7 genes of ABCC4 (SEQ ID NO:1), CCL19 (SEQ ID NO:2), SLC39A8 (SEQ ID NO:3), CD27 (SEQ ID NO:4), FUT7 (SEQ ID NO:5), ZNF71 (SEQ ID NO:6), and DAG1 (SEQ ID NO:7), normalizing of the quantification of the 7 genes with the quantification of a control gene UBC (SEQ ID NO:8) or a housekeeping gene, and utilizing the normalized 7 gene mRNA expression quantification to determine whether the patient will benefit from receiving adjuvant chemotherapy or not.

### CROSS-REFERENCE TO RELATED APPLICATION

This patent application claims the benefit of co-pending PCT/US2019/036953, filed, Jun. 13, 2019. The entire contents of PCT/US2019/036953 is incorporated by reference into this patent application as if fully written herein.

### STATEMENT REGARDING FEDERALLY SPONSORED RESEARCH OR DEVELOPMENT

This invention was made with government support under National Institute of Health Grants RO1 LM009500, R56 LM009500, RO1 ES021764, and P20 RR016440. The government has certain rights in the invention.

### SEQUENCE LISTING

A SEQUENCE LISTING in computer-readable form (.txt file) accompanies this application having SEQ ID NO:1 through SEQ ID NO:10. The computer-readable form (.txt file) of the SEQUENCE LISTING is incorporated by reference into this application.

### BACKGROUND OF THE INVENTION

### 1. Field of the Invention

This invention relates to a method of providing a treatment to a patient having non-small cell lung cancer comprising extracting total RNA from a formalin fixed and paraffin embedded tumor of non-small cell lung cancer of a patient after the surgical resection, generating complementary DNA (cDNA) of the extracted total RNA from said patient tumor, quantifying of mRNA expression of 7 genes of ABCC4 (SEQ ID NO:1), CCL19 (SEQ ID NO:2), SLC39A8 (SEQ ID NO:3), CD27 (SEQ ID NO:4), FUT7 (SEQ ID NO:5), ZNF71 (SEQ ID NO:6); and DAG1 (SEQ ID NO:7), normalizing of the quantification of said 7 genes with the quantification of a control gene UBC (SEQ ID NO:8) or a housekeeping gene, and utilizing said normalized 7 gene mRNA expression quantification to determine whether said patient will benefit from receiving adjuvant chemotherapy or not. This invention also relates to a method of providing a treatment to a patient having non-small cell lung cancer comprising providing protein expression of SEQ ID NO:9 (protein ZNF71) or SEQ ID NO:10 (protein CD27) and quantifying said protein expression with ELISA, or immunocytochemistry staining or immunohistochemistry staining correlated with either said CD27 mRNA expression or ZNF71 mRNA, respectively, in a patient tumor and a cancer-free tissue adjacent to said tumor, and determining a prognosis of said patient from said protein expression.

### 2. Background Art

Lung cancer is the leading cause of cancer-related deaths in the world, and non-small cell lung cancer (NSCLC) accounts for almost 80% of lung cancer deaths [1]. Major histology of NSCLC includes lung adenocarcinoma and squamous cell lung carcinoma. Surgical resection is the major treatment for early stage NSCLC. However, about 22-38% of stage I NSCLC patients will develop tumor recurrence within five years following the surgery [2]. It is therefore important to select early stage NSCLC patients for more aggressive treatment. While adjuvant chemotherapy of stage II and stage III disease has resulted in 10-15% increased overall survival [3], the prognosis for early stage NSCLC remains poor [4]. Currently, there are no clinically available molecular assays to predict the risk for tumor recurrence and the clinical benefits of chemotherapy in NSCLC patients.

Immunotherapy has rapidly gained attention of oncologists as an effective and less toxic treatment than chemotherapy in patients with advanced lung cancers [5-7]. A recent study used paired single cell analysis to compare normal lung tissue and blood with tumor tissue in stage I NSCLC, and found that early-stage tumors had already begun to alter the immune cells in their microenvironment [8]. These results suggest that immunotherapy could potentially be used to treat early stage lung cancer patients. However, predictive biomarkers of immunotherapy are not well established except PD-1/PD-L1, and it is unlikely that a single marker is sufficient.

High-throughput technologies, such as microarray and RNA-seq, promise the discovery of novel biomarkers from genome-scale studies. The FDA conducted a systematic evaluation and suggested continued usefulness of legacy microarray data and established microarray biomarkers and predictive models in the forthcoming RNA-seq era [9]. However, several disadvantages have limited the application of high-throughput techniques in routine clinical tests, including costs, reproducibility, and data analyses [10]. Compared with microarray/RNA-seq, quantitative real-time RT-PCR (qRT-PCR) is more efficient, consistent, and able to measure gene expression over a greater dynamic range [11]. The combined use of real-time qRT-PCR with high-throughput analysis can overcome the inherent biases of the high-throughput techniques and is emerging as the optimal method of choice to translate genome research into clinical practice [12]. The protein expression validation of the identified MRNA biomarkers could substantiate their ultimate functional involvements in disease, and may lead to the discovery of potential proteomic biomarkers in abundant FFPE samples for broader applications in community hospitals.

DNA microarray-based studies identified gene expression-based NSCLC prognostic [13] and predictive biomarkers [14, 15]. A qRT-PCR based 14-gene assay by Kratz et al [16] is prognostic of non-squamous NSCLC outcome in FFPE tissues and is ready for wide-spread clinical applications. However, this 14-gene assay is limited to non-squamous NSCLC and is not shown to be predictive of the clinical benefits of chemotherapy.

### SUMMARY OF THE INVENTION

The present invention provides a multi-gene assay predictive of the clinical benefits of chemotherapy in non-small cell lung cancer (NSCLC) patients, and provides for their protein expression as therapeutic targets.

This invention discloses a method using a 7-gene assay ABCC4 (SEQ ID NO:1), CCL19 (SEQ ID NO:2), SLC39A8 (SEQ ID NO:3), CD27 (SEQ ID NO:4), FUT7 (SEQ ID NO:5), ZNF71 (SEQ ID NO:6), and DAG1(SEQ ID NO:7) for selecting adjuvant chemotherapy treatment for a patient with non-small cell lung cancer after their surgery. This treatment method using the 7-gene assay can predict a patient's formalin fixed and paraffin embedded tumor as either with benefit from adjuvant chemotherapy or no benefit from adjuvant chemotherapy after receiving surgery. In the published data of the 7-gene assay, it is shown that for those patients who were predicted as with benefit from chemotherapy, their disease specific-survival was significantly (p<0.05) longer in those who actually received adjuvant chemotherapy compared with those who did not receive adjuvant chemotherapy. In the contrast, for those patients who were predicted with the 7-gene assay as no benefit from adjuvant chemotherapy, their disease-specific survival was actually shorter when they received adjuvant chemotherapy compared with those who did not receive adjuvant chemotherapy, due to unnecessary chemotherapeutic treatment and associated cytotoxicity side-effects (see FIG. 1). The adjuvant chemotherapy included in the studied patient cohorts comprises Cisplatin and Taxol, Cisplatin and Taxotere, Carboplatin, Carboplatin and Taxol, Carboplatin and Taxotere, Taxol, and Alimta (pemetrexed).

Within the 7-gene assay, 4 genes, ABCC4 (SEQ ID NO:1), FUT7 (SEQ ID NO:5), ZNF71 (SEQ ID NO:6), and SLC39A8 (SEQ ID NO:3), each individually predicted chemosensitivity or chemoresistance to specific adjuvant chemotherapy (see Table 2). Specifically, high expression of ABCC4 (SEQ ID NO:1) predicted chemoresistance to Carboplatin and Taxol, Taxol, Carboplatin and Taxotere, Cisplatin and Taxetere, and Cisplatin and Taxol. High expression of FUT7 (SEQ ID NO:5) predicted chemosensitivity to Carboplatin. High expression of ZNF71 (SEQ ID NO:6) predicted chemosentivity to Carboplatin and Taxol, Carboplatin and Taxotere, Cisplatin and Taxotere, and Cisplatin and Taxol. High expression of SLC39A8 (SEQ ID NO:3) predicted chemoresistance to Taxol and Alimta (pemetrexed).

The protein expression of ZNF71 (SEQ ID NO:9) quantified with automated quantitative analysis (AQUA) correlated with its mRNA expression in patient tumors. The protein expression of ZNF71 (SEQ ID NO:9) can independently classify patients into prognosis (longer survival) group or poor prognosis (shorter survival) group (see FIG. 2).

The protein expression of CD27 (SEQ ID NO:10) quantified with ELISA had a significant correlation with its mRNA in patient tumors and adjacent normal lung tissues, and could be an independent protein biomarker for patient prognosis and treatment selection (see FIG. 3).

An embodiment of this invention provides a method of providing a treatment to a patient having non-small cell lung cancer comprising extracting total RNA from a formalin fixed and paraffin embedded tumor of non-small cell lung cancer of a patient after the surgical resection; generating complementary DNA (cDNA) of the extracted total RNA from said patient tumor; quantifying of mRNA expression of 7 genes of ABCC4 (SEQ ID NO:1), CCL19 (SEQ ID NO:2), SLC39A8 (SEQ ID NO:3), CD27 (SEQ ID NO:4), FUT7 (SEQ ID NO:5), ZNF71 (SEQ ID NO:6); and DAG1 (SEQ ID NO:7); normalizing of the quantification of said 7 genes with the quantification of a control gene UBC (SEQ ID NO:8) or a housekeeping gene; and utilizing said normalized 7 gene mRNA expression quantification to determine whether said patient will benefit from receiving adjuvant chemotherapy or not. In a preferred embodiment of this method, the method further comprises administering to said patient a therapeutically effective amount of one of the following adjuvant chemotherapies (a) Cisplatin and Taxol, (b) Cisplatin and Taxotere, (c) Carboplatin, (d) Carboplatin and Taxol, (e) Carboplatin and Taxotere, (f) Taxol, and (g) Alimta (pemetrexed). In a more preferred embodiment of tis method, this method comprises the quantification of mRNA expression of three genes of ABCC4 (SEQ ID NO:1), CCL19 (SEQ ID NO:2), and SLC39A8 (SEQ ID NO:3). In another preferred embodiment of this invention, this method comprises the quantification of mRNA expression of four genes of CD27 (SEQ ID NO:4), FUT7 (SEQ ID NO:5), ZNF71 (SEQ ID NO:6), and DAG1 (SEQ ID NO:7).

Another embodiment of this invention provides the method, as described above, wherein said quantification of mRNA expression of ABCC4 (SEQ ID NO:1) and utilization of said normalized ABCC4 gene mRNA expression quantification to determine whether said patient will benefit from receiving adjuvant chemotherapy of one of (a) Cisplatin and Taxol, (b) Cisplatin and Taxotere, (d) Carboplatin and Taxol, (e) Carboplatin and Taxotere, and (f) Taxol.

Another embodiment of this invention provides the method, as described above, wherein said quantification of mRNA expression of FUT7 (SEQ ID NO:5) and utilization of said normalized FUT7 gene mRNA expression quantification to determine whether said patient will benefit from receiving adjuvant chemotherapy of Carboplatin.

Another embodiment of this invention provides the method, as described above, wherein said quantification of mRNA expression of ZNF71 (SEQ ID NO:6) and utilization of said normalized ZNF71 gene mRNA expression quantification to determine whether said patient will benefit from receiving adjuvant chemotherapy of (a) Carboplatin and Taxol, (b) Carboplatin and Taxotere, (c) Cisplatin and Taxotere, and (d) Cisplatin and Taxol.

Another embodiment of this invention provides the method, as described above, wherein said quantification of mRNA expression of SLC39A8 (SEQ ID NO:3) and utilization of said normalized SLC39A8 gene mRNA expression quantification to determine whether said patient will benefit from receiving adjuvant chemotherapy of one of (a) Taxol, and (b) Alimta (pemetrexed).

In another embodiment of this invention a method of providing a treatment to a patient having non-small cell lung cancer, is disclosed, comprising providing protein expression of ZNF71 (SEQ ID NO: 9); quantifying said protein expression of said ZNF71 (SEQ ID NO:9) with automated quantitative analysis (AQUA) correlated with said ZNF71 (SEQ ID NO:9) mRNA expression in a patient tumor; and determining a prognosis of said patient from said protein expression of said ZNF71 (SEQ ID NO:9). In another embodiment of this method, the method includes wherein said prognosis of said patient is either longer survival or shorter survival.

In another embodiment of this invention, a method of providing a treatment to a patient having non-small cell lung cancer, is disclosed, comprising providing protein expression of CD27 (SEQ ID NO:10); quantifying said protein expression of said CD27 (SEQ ID NO:10) with ELISA correlated with said CD27 (SEQ ID NO:10) mRNA expression in a patient tumor and a cancer-free tissue adjacent to said tumor; and determining a prognosis of said patient from said protein expression of said CD27 (SEQ ID NO:10). In another embodiment of this method, the method includes wherein said prognosis of said patient is either longer survival or shorter survival. This method, optionally, includes administering to said patient a therapeutically effective amount of an adjuvant chemotherapy.

### BRIEF DESCRIPTION OF THE DRAWINGS

FIG. 1A shows a patient stratification in training cohort CWRU of a Kaplan-Meier analyses of the 7-gene model of this invention.

FIG. 1B shows a CWRU high-risk group of a Kaplan-Meier analyses of the 7-gene model of this invention.

FIG. 1C shows a CWRU low-risk group of a Kaplan-Meier analyses of the 7-gene model of this invention.

FIG. 1D shows a validation set of a Kaplan-Meier analyses of the 7-gene model of this invention.

FIG. 1E shows a validation set high-risk group of a Kaplan-Meier analyses of the 7-gene model of this invention.

FIG. 1F shows a validation set low-risk group of a Kaplan-Meier analyses of the 7-gene model of this invention.

FIG. 2A shows a Kaplan-Meier analyses of ZNF7I (SEQ ID NO:9) protein expression quantified by AQUA, wherein ZNF71 (SEQ ID NO:9) immunofluorescence images of different expression levels in TMA.

FIG. 2B shows patients were stratified into two groups based on ZNF71 (SEQ ID NO:9) AQUA scores. Patients with logₑ(ZNF7 ((SEQ NO:9)) AQUA Score)>7.9 had a low-risk and those with logₑ(ZNF71 ((SEQ ID NO:9)) AQUA Score)<7.9 had a high-risk for tumor metastasis in training cohort YTMA250.

FIG. 2C shows a validation cohort YTMA79. P values were assessed with Wilcoxon tests.

FIG. 3A shows a comparison of mRNA and protein expression of CD27 (SEQ ID NO:10) in NSCLC patient samples, wherein a scatterplot with regression line for CD27 mRNA (relative quantity) in qRT-PCR and protein expression (pg/mL) in ELISA assays of 29 NSCLC tumor resections. RQ: relative quantity, measured as 2^{Act} values in qRT-PCR with UBC as the control gene. R: Spearman correlation coefficient.

FIG. 3B shows a comparison of CD27 (SEQ ID NO:10) fold-change in NSCLC vs. normal lung tissues and high-risk vs. low-risk NSCLC tumors in qRT-PCR and ELISA assays. High-risk NSCLC patients had a poor survival outcome and low-risk NSCLC patients had a good survival outcome. Bar plot shows mean +SE, *: P<0.05.

FIG. 4A shows the 7-gene prognostic and predictive NSCLC model wherein the 7-gene model is in decision-tree format.

FIG. 4B shows the 7-gene prognostic and predictive model in rule-base format.

FIG. 5A shows the molecular network and pathway analysis in Ingenuity Pathway Analysis (IPA), namely, top molecular network of 7 NSCLC biomarkers in IPA analysis.

FIG. 5B shows the top molecular pathways of the 7-gene signature of this invention in IPA analysis.

FIG. 6 shows DNA copy number variation of the 7 signature genes of this invention in NSCLC (n=271), The DNA copy number data is available in NCBI Gene Expression Omnibus with accession number GSE31800. The CGHCall package in R was used in the analysis.

FIG. 7A shows immunohistochemistry (IHC) staining results of ZNF71 in NSCLC patient FFPE samples (n=24) of the method of this invention.

FIG. 7B shows a summary of distribution of ZNF71 IHC scores in the study cohort of the method of this invention.

FIG. 8 shows an example of output from the web-based version of the comprehensive prognostic model PersonalizedRx. Given the patient information submitted by the user (left), the web-based tool will estimate survival for each treatment category using the survival observed for patients of a particular treatment modality and similar Hazard Score (right).

FIG. 9 shows that NSCLC patients without COPD showed consistently and significantly better survival when compared to those with COPD across the entire period of post-operative follow-up, indicating that the effects of COPD are manifested in both long- and short-term disease-specific survival. FIG. 9 shows a Kaplan-Meier analysis of patients with and without COPD among those treated with surgery alone. Log-rank tests were used to assess the difference in survival probabilities of two groups.

FIG. 10 shows a comprehensive prognostic model for lung adenocarcinoma, AJCC 3^{rd} Staging Edition. Patient survival at 60 months for the total population sample is shown for the range of Hazard Scores (left), with the risk-groups delimited by vertical bars. Model coefficients used to determine the Hazard Score for each patient are shown on the forest plot (right).

FIG. 11 shows a comprehensive prognostic model for lung adenocarcinoma, AJCC 6^{th} Staging Edition. Patient survival at 60 months for the total population sample is shown for the range of Hazard Scores (left), with the risk-groups delimited by vertical bars. Model coefficients used to determine the Hazard Score for each patient are shown on the forest plot (right).

FIG. 12 shows a comprehensive prognostic model for lung adenocarcinoma, AJCC 7^{th} Staging Edition. Patient survival at 60 months for the total population sample is shown for the range of Hazard Scores (left), with the risk-groups delimited by vertical bars. Model coefficients used to determine the Hazard Score for each patient are shown on the forest plot (right).

FIG. 13 shows a comprehensive model for squamous cell lung carcinoma, AJCC 3^{rd} Staging Edition. Model coefficients used to determine the Hazard Score for each patient are shown on the forest plot (right). Patient survival at 24 months for the total population sample is shown for the range of Hazard Scores (left), with the risk-groups delimited by vertical bars.

FIG. 14 shows a comprehensive model for squamous cell lung carcinoma, AJCC 6^{th} Staging Edition. Model coefficients used to determine the Hazard Score for each patient are shown on the forest plot (right). Patient survival at 24 months for the total population sample is shown for the range of Hazard Scores (left), with the risk-groups delimited by vertical bars.

FIG. 15 shows a comprehensive model for squamous cell lung carcinoma, AJCC 7^{th} Staging Edition. Model coefficients used to determine the Hazard Score for each patient are shown on the forest plot (right). Patient survival at 24 months for the total population sample is shown for the range of Hazard Scores (left), with the risk-groups delimited by vertical bars.

FIG. 16 shows the effect of COPD in Adenocarcinoma AJCC 3^{rd} Edition stage and treatment sub-groups. For each group, a clear and significant difference between the survival curves for patients with and without COPD can be seen, with patients identified as having COPD experiencing significantly poorer survival compared to those without COPD.

FIG. 17 shows the effect of COPD in Adenocarcinoma AJCC 6^{th} Edition stage and treatment sub-groups. For each group, patients without COPD tend to experience longer survival when compared to patients with COPD, although the difference is not significant in some cases shown.

FIG. 18 shows the effect of COPD in Adenocarcinoma AJCC 7^{th} Edition stage and treatment sub-groups. For each group treated without chemotherapy, a clear and significant difference between the survival curves for patients with and without COPD can be seen, with patients identified as having COPD experiencing significantly poorer survival compared to those without the disease. The difference in survival for patients treated with systemic therapy was not significant, but trended toward patients with COPD having poorer survival.

FIG. 19 shows the effect of COPD in Squamous Cell AJCC 3^{rd} Edition stage and treatment sub-groups. For each group, a clear and significant difference between the survival curves for patients with and without COPD can be seen, with patients identified as having COPD experiencing significantly poorer survival compared to those without the disease.

FIG. 20 shows the effect of COPD in Squamous Cell AJCC 6^{th} Edition stage and treatment sub-groups. For the group shown, a clear and significant difference between the survival curves for patients with and without COPD can be seen, with patients identified as having COPD experiencing significantly poorer survival compared to those without the disease.

FIG. 21 shows the effect of COPD in Squamous Cell AJCC 7^{th} Edition stage and treatment sub-groups. For each group, a clear and significant difference between the survival curves for patients with and without COPD can be seen, with patients identified as having COPD experiencing significantly poorer survival compared to those without the disease.

FIG. 22 shows improvement in the Full model using COPD over Stage Alone. For each Kaplan-Meier plot, the three pairs of lines represent the High, Intermediate, and Low-Risk groups defined for each of the two models shown. The model using only the AJCC Stage is shown in orange color, while the Full model with COPD status added is shown in blue color. For each plot shown, the Full model with COPD status was able to produce a Low-Risk group with better survival and a High-Risk group with poorer survival, with most cases being significant (p<0.05).

### DETAILED DESCRIPTION OF THE INVENTION

PCT/US2019/036953 discloses a 7-gene assay using snap-frozen samples to identify patients at risk for tumor recurrence and metastasis and selection of optimal chemotherapeutic regimen in these patients. The following 7 genes were used in the gene assay based on qRT-PCR: ABCC4, CCL19, CD27, DAG1, FUT7, SLC39A8, and ZNF44. In this invention, a new protocol and algorithm for this new 7-gene assay in non-small lung cancer patient formalin fixed samples and paraffin embedded samples is provided. The present method comprises extracting total RNA from patient formalin fixed and patient paraffin embedded samples; quantifying mRNA expression profiles in qRT-PCR in formalin fixed and paraffin embedded samples and then compared with the matched snap-frozen tumor tissues from the same patient; based on the collected data a new algorithm and methodology is developed for prognosis and prediction of chemotherapy benefits in non-small cell lung cancer patients using the patient's formalin fixed and paraffin embedded sample. Protein expression of the identified biomarkers was also evaluated in patient formalin fixed or paraffin embedded tissue samples using immunohistochemistry (IHC). IHC is commonly used in pathology laboratories, and the IHC results of the method of the present invention provide prognosis of non-small lung cancer as independent companion tests. Immunohistochemistry is the most common application of immunostaining. IHC is a known technique used to determine the presence and level of specific cellular proteins. IHC involves the process of selectively identifying antigens (proteins) in cells of a tissue section by exploiting the principle of antibodies binding specifically to antigens in a biological tissue, here non-small cell lung cancer tissue. In general, immunohistochemistry comprises the the following steps: (1) fixation to keep the sample in place, (2) antigen retrieval to increase the availablility of proteinsfor detection, (3) bocking to minimize any background signals, and (4) antibody labeling and visualization. Generally, immunohistochemistry markers are monoclonal antibodies used to identify specific proteins in tissue samples. The antibody binds to the protein and a color reagent stains the protein, if in fact that protein is present in the tissue sample.

Specifically this invention provides ZNF71 protein expression in formalin fixed and paraffin embedded samples was a prognostic biobarker of non-small cell lung cancer using a technique known by those skilled in the art as AQUA. In this method, we use quantification results from IHC tests with new antibodies for ZNF71. This invention provides new protein expression assays for ZNF71 for non-small cell lung cancer prognosis. In addition CD27 was previously reported as a potential protein biomarker based upon snap-frozen samples (PCT/US2019/036953). This invention tests CD27 in formalin fixed and paraffin embedded samples with immunocytochemistry staining. This invention provides a prognostic model for non-small cell lung cancer using patient clinical, pathological, and demographic information to inform optimal treatment options. An online tool, PersonalizeRX (available at www.personalizedrx.org) is already used in clinics worldwide. This method integrates the mRNA 7-gene assay, protein based IHC tests, and the PersonalizeRX tool, into one algorithm to provide healthcare providers (i.e. physicians and clinicians) with an accurate estimate of a non-small cell lung cancer patient clinical outcomes. This method thus provides a tool for establishing precision therapy in non-small cell lung cancer patients.

PCT/US2019/036953 describes a 7-gene assay based on snap-frozen non-small cell lung cancer patient samples. The technology described in PCT/US2019/036953 is not applicable to formalin fixed or paraffin embedded samples that are abundant in the majority of community hospitals. PCT/US2019/036953 describes a protein biomarker ZNF71 based upon AQUA and a now discontinued antibody. The present invention provides a method to quantify ZNF71 with new antibodies using IHC in formalin fixed and paraffin embedded samples of non-small cell lung cancer tumors. Further, this 7-gene assay of the present invention and the IHC assay of ZNF71 is integrated with patient clinical, pathologies, and demographic information into one algorithm for selection of optimal treatment of the non-small cell lung cancer patient. The present invention provides a method that utilizes formalin fixed and paraffin embedded non-small cell lung cancer patient tissue samples for mRNA quantification.

This invention provides a (1) a mRNA based 7-gene assay and algorithm, (2) an IHC based ZNF71 and CD27 assays and algorithm, and (3) an integrated mRNA 7-gene assay, ZNF71 and CD27 IHC assays, and patient clinical information in one algorithm. Materials:

122 non-small cell lung cancer patient samples were obtained form Case Western Reserve University, 101 lung adenocarcinoma tumor specimens from University of Michigan Comprehensive Cancer Center, 65 non-small cell lung cancer tumor specimens from NorthShore University Health System Kellogg Center Cancer Center, and 49 specinens from West Virginia University Cancer Institute/Mary Babb Randolph Cancer Center.

Alexa 546-conjugated goat anti-mouse seconday antibody (Life Technologies), Cy5-Tyramide (Perkin Elmer).

Human CD27/TNFSRF7 DuoSet ELISA kit (contained antibodies).

ZNF71 antibodies from GeneTex and Sigma.

Data from JBR.10 clinical trial were obtained from the NCBI Gene Expression Omnibus website.

An embodiment of this invention provides a method of providing a treatment to a patient having non-small cell lung cancer comprising extracting total RNA from a formalin fixed or paraffin embedded tumor of non-small cell lung cancer of a patient after the surgical resection; generating complementary DNA (cDNA) of the extracted total RNA from said patient tumor; quantifying of mRNA expression of 7 genes of ABCC4 (SEQ ID NO:1), CCL19 (SEQ ID NO:2), SLC39A8 (SEQ ID NO:3), CD27 (SEQ ID NO:4), FUT7 (SEQ ID NO:5), ZNF71 (SEQ ID NO:6); and DAG1 (SEQ ID NO:7); normalizing of the quantification of said 7 genes with the quantification of a control gene UBC (SEQ ID NO:8) or a housekeeping gene; and utilizing said normalized 7 gene mRNA expression quantification to determine whether said patient will benefit from receiving adjuvant chemotherapy or not.

As used herein, a housekeeping gene is a typically constitutive genes that is required for the maintenance of basal cellular functions that are essential for the existence of a cell, regardless of its specific role in the tissue or organism. Thus, they are generally expressed in all cells of an organism under normal and patho-physiological conditions, irrespective of tissue type, developmental stage, cell cycle state, or external signal. For example, housekeeping genes are widely used as internal controls for experimental studies. The reliability of any relative RT-PCR experiment can be improved by including an invariant endogenous control (reference gene) in the assay to correct for sample to sample variations in RT-PCR efficiency and errors in sample quantification. A biologically meaningful reporting of target mRNA copy numbers requires accurate and relevant normalization to some standard and is recommended in quantitative RT-PCR. Many housekeeping genes are known to those persons skilled in the art, such as for example, but not limited to, 18S ribosomal RNA (RRN18S), RNA polymerase 2 subunit A (PolR2A), glyceraldehyde phosphate dehydrogenase (GAPDH), or β 2-microglobulin (B2M).

In a preferred embodiment of this method, the method further comprises administering to said patient a therapeutically effective amount of one of the following adjuvant chemotherapies (a) cisplatin and Taxol (paclitaxel), (b) cisplatin and Taxotere (docetaxel), (c) carboplatin, (d) carboplatin and Taxol, (e) carboplatin and Taxotere, (f) Taxol, and (g) Alimta (pemetrexed). Taxol is a registered trademark owned by Bristol-Myers Squibb Company, New York, N.Y., USA; Taxotere is a registered trademark owned by Aventis Pharma S.A., Cedex, France; and Alimnta is a registered trademark owned by Eli Lilly and Company, Indianapolis, Ind., USA. In a more preferred embodiment of this method, this method comprises the quantification of mRNA expression of three genes of ABCC4 (SEQ ID NO:1), CCL19 (SEQ ID NO:2), and SLC39A8 (SEQ ID NO:3). In another more preferred embodiment of this method, this method comprises the quantification of mRNA expression of four genes of CD27 (SEQ ID NO:4), FUT7 (SEQ ID NO:5), ZNF71 (SEQ ID NO:6), and DAG1 (SEQ ID NO:7).

Another embodiment of this invention provides the method, as described above, wherein said quantification of mRNA expression of ABCC4 (SEQ ID NO:1) and utilization of said normalized ABCC4 (SEQ ID NO:1) gene mRNA expression quantification to determine whether said patient will benefit from receiving adjuvant chemotherapy of one of (a) cisplatin and Taxol, (b) cisplatin and Taxotere, (d) carboplatin and Taxol, (e) carboplatin and Taxotere, and (f) Taxol.

Another embodiment of this invention provides the method, as described above, wherein said quantification of mRNA expression of FUT7 (SEQ ID NO:5) and utilization of said normalized FUT7 (SEQ ID NO:5) gene mRNA expression quantification to determine whether said patient will benefit from receiving adjuvant chemotherapy of Carboplatin.

Another embodiment of this invention provides the method, as described above, wherein said quantification of mRNA expression of ZNF71 (SEQ ID NO:6) and utilization of said normalized ZNF71 (SEQ ID NO:6) gene mRNA expression quantification to determine whether said patient will benefit from receiving adjuvant chemotherapy of (a) carboplatin and Taxol, (b) carboplatin and Taxotere, (c) cisplatin and Taxotere, and (d) cisplatin and Taxol.

Another embodiment of this invention provides the method, as described above, wherein said quantification of mRNA expression of SLC39A8 (SEQ ID NO:3) and utilization of said normalized SLC39A8 (SEQ ID NO:3) gene mRNA expression quantification to determine whether said patient will benefit from receiving adjuvant chemotherapy of one of (a) Taxol, and (b) Alimta (pemetrexed).

In another embodiment of this invention a method of providing a treatment to a patient having non-small cell lung cancer, is disclosed, comprising providing protein expression of ZNF71 (SEQ ID NO: 9); quantifying said protein expression of said ZNF71 (SEQ ID NO:9) with automated quantitative analysis (AQUA) correlated with said ZNF71 mRNA expression in a patient tumor; and determining a prognosis of said patient from said protein expression of said ZNF71 (SEQ ID NO:9). In another embodiment of this method, the method includes wherein said prognosis of said patient is either longer survival or shorter survival.

In another embodiment of this invention, a method of providing a treatment to a patient having non-small cell lung cancer, is disclosed, comprising providing protein expression of CD27 (SEQ ID NO:10); quantifying said protein expression of said CD27 (SEQ ID NO:10) with ELISA correlated with said CD27 mRNA expression in a patient tumor and a cancer-free tissue adjacent to said tumor; and determining a prognosis of said patient from said protein expression of said CD27 (SEQ ID NO:10). In another embodiment of this method, the method includes wherein said prognosis of said patient is either longer survival or shorter survival. This method further includes administering to said patient a therapeutically effective amount of an adjuvant chemotherapy.

Patients and methods: The mRNA expression of 160 genes identified from microarray was analyzed in qRT-PCR assays of independent formalin fixed and paraffin embedded (“FFPE”) non-small cell lung cancer (NSCLC) tumors to develop a predictive signature. A clinical trial JBR.10 was included in the validation. Hazard ratio was used to select genes, and decision-trees were used to construct the predictive model. Protein expression was quantified with AQUA in 500 FFPE NSCLC samples. Results: A 7-gene signature (of this invention) was identified from training cohort (n=83) with accurate patient stratification (P=0.0043) and was validated in independent patient cohorts (n=248, P<0.0001) in Kaplan-Meier analyses. In the predicted benefit group, there was a significantly better disease-specific survival in patients receiving adjuvant chemotherapy in both training (P=0.035) and validation (P=0.0049) sets. In the predicted non-benefit group, there was no survival benefit in patients receiving chemotherapy in either set. The protein expression of ZNF71 (SEQ ID NO:9) quantified with AQUA scores produced robust patient stratification in separate training (P=0.021) and validation (P=0.047)NSCLC cohorts. The protein expression of CD27 (SEQ ID NO:10) quantified with ELISA had a strong correlation with its mRNA expression in NSCLC tumors (Spearman coefficient-0.494, P<0.0088). Multiple signature genes had concordant DNA copy number variation, mRNA and protein expression in NSCLC progression.

Those persons skilled in the art will understand that this invention presents a predictive multi-gene assay and prognostic protein biomarkers clinically applicable for improving NSCLC treatment in patients suing formalin fixed or paraffin embedded tumor samples, with important implications in lung cancer chemotherapy/immunotherapy.

In this invention, a combined analysis of genome-wide transcriptional profiles and qRT-PCR was utilized to develop a multi-gene assay both prognostic of NSCLC outcome and predictive of the benefits of chemotherapy. Patient cohorts from multiple hospitals in the US and JBR.10 data [14] were used to validate this multi-gene assay. Protein expression of the identified biomarkers (using immunohistochemistrywas also evaluated in patient tissue samples and correlated with the mRNA expression and DNA copy number variation to substantiate their functional involvement and potential as therapeutic targets in chemotherapy and immunotherapy, in addition to companion tests.

Materials and Methods

Patient samples. Clinical characteristics of patient cohorts used in qRT-PCR assays are summarized in Table 1. All NSCLC patients were staged I, II, or IIIA at the time of diagnosis. Tumor tissues were collected in surgical resections and were then formalin fixed or paraffin embedded until used for RNA extraction. Tumor cell content was above 50% for qRT-PCR assays. Those with missing AJCC staging information, missing histology, death within 30 days of resection or from other disease conditions were excluded from further analysis. A total of 122 NSCLC patient samples were obtained from Case Western Reserve University (CWRU) Comprehensive Cancer Center. Total RNA of good quality was extracted from 89 tumor specimens. Good quality RNA from 101 lung adenocarcinoma tumor specimens was obtained from University of Michigan (UM) Comprehensive Cancer Center, with detailed description of patients, tissue specimens and mRNA quality check provided in [17]. A total of 65 NSCLC tumor specimens from NorthShore University HealthSystem Kellogg Cancer Center and 49 specimens from West Virginia University Cancer Institute [Mary Babb Randolph Cancer Center (MBRCC)] generated good quality mRNA. The tissue collection in this study was approved by an Institutional Review Board (IRB) at each institution.

RNA extraction, and quality and concentration assessments. Total RNA was extracted from formalin fixed or paraffin embedded tumor tissues using a RNeasy mini kit according the manufacturer's protocol (Qiagen, USA), followed by elution in 30 μl of RNase-free water and storage at −80° C. The quality and integrity of the RNA, the 28S to 18S ratio, and a visual image of the 28S and 18S bands were evaluated on the 2100 Bioanalyzer (Agilent Technologies, CA). RNA assessed as having good quality from 304 tumor samples was included for further analysis. The RNA concentration of each sample was assessed using a Nanodrop-1000 Spectrophotometer (NanoDrop Tech, Germany).

Generation of complementary DNA (cDNA). The reverse transcriptase polymerase chain reaction was used to convert the high-quality single-stranded RNA samples to double-stranded cDNA, using an Applied Biosystems GeneAmp® PCR 9700 machine (Foster City, Calif.). For standardization across all samples, one microgram of RNA was used to generate cDNA.

Real-time quantitative RT-PCR low-density arrays. Real-time qRT-PCR assays of independent patient cohorts of NSCLC tumor samples were used to further select biomarkers to form a multi-gene assay from prognostic genes identified from microarray data in our previous studies [18-21]. The identified prognostic genes were initially validated with multiple independent NSCLC microarray data publically available [18-21]. Based on the validation results, 160 prognostic genes and three housekeeping genes were included in the qRT-PCR experiments. The three housekeeping genes were 18S, UBC, and POLR2A due to their confirmed constant mRNA expressions across samples [18].

Three hundred thirty seven (337) tumor samples were analyzed with good RNA quality using TaqMan microfluidic low-density array (LDA) plates on an ABI 7900HT Fast RT-PCR instrument (Applied Biosystems). Total RNA samples were analyzed on an Agilent 2100 Bioanalyzer RNA 6000 Nano LabChip. The report was generated by the SDS2.3 software (Applied Biosystems). In the report, the number of cycles required to reach threshold fluorescence (Ct) and ΔCT for each sample relative to the control gene defines the expression pattern for a gene. The gene expression data were further analyzed using the 2^{ΔΔC}^{_{T}} method [22].

Statistical and computational analysis. Prognostic biomarkers were evaluated with Cox proportional hazard model. Hazard ratio was used in the evaluation of prognostic performance of biomarkers. If a biomarker gives a hazard ratio greater than 1, it means that patient samples predicted as high risk are more likely to have a poor outcome. In the evaluation of genes in qRT-PCR assays, ΔCT was used as a covariate in Cox model. If a gene as a hazard ratio greater than 1, it means that down-regulation of this gene is associated with a poor outcome and up-regulation of this gene is associated with a good outcome in NSCLC patients; otherwise, if a gene has a hazard ratio less than 1, it means that down-regulation of this gene is associated with a good outcome and up-regulation of this gene is associated with a poor outcome in NSCLC patients. During the evaluation, UBC (Hs00824723_ml) was chosen as the house keeping gene to normalize gene expression. The CWRU cohort was used as the training set, and seven genes were selected to form a prognostic classifier based on decision trees. These seven genes are ABCC4 (Hs00988717_ml) (SEQ ID NO:1), CCL19 (Hs00171149_ml) (SEQ ID NO:2), SLC39A8 (Hs00223357_ml) (SEQ IDNO:3), CD27 (Hs00154297_ml) (SEQ ID NO:4), FUT7 (Hs00237083_ml) (SEQ ID NO:5), ZNF71 (Hs00221893_ml) (SEQ ID NO:6), and DAG1 (Hs00189308_ml) (SEQ ID NO:7). The 7-gene prognostic method of this invention was validated with independent patient cohorts (UM, MBRCC, and NorthShore). In Kaplan-Meier analysis, log-rank tests or Wilcoxon tests were used to assess the difference in probability of survival of different prognostic groups. All the analyses were performed with packages in R or SAS unless otherwise specified.

Validation on clinical trial JBR.10. Data from JBR.10 was obtained from NCBI Gene Expression Omnibus with accession number GSE14814. A total of 133 non-small cell lung cancer samples were profiled for gene expression using Affymetrix 133A platform [14]. Patients were all in early stage (I or II). Patient samples assayed in the same batch with consecutive accession numbers ranging from GSM370913 to GSM371002 (n=90) were used in the validation of the 7-gene signature. Among these patient samples, those who died from other disease conditions were excluded from further analysis. ABCC4 (203196_at), CCL19 (210072_at), CD27 (206150_at), DAG1 (205417_s_at and 212128_s_at), FUT7 (210506_at and 217696_at), SLC39A8 (209266_s_at, 209267_s_at, 216504_s_at, and 219869_s_at), and ZNF444 (218707_at and 50376_at) were used in validating the qRT-PCR based multi-gene assay. For a gene with multiple probe sets, the one with the highest expression value (yielding the clearest signal) in each sample was chosen to represent the gene expression. ZNF71 was not available in the GSE14814 dataset. ZNF444 was chosen to replace ZNF71 to validate the qRT-PCR results, because both ZNF444 and ZNF71 are at locus NC 000019.10 in Chromosome 19 and belong to zinc finger protein family. To be compatible with the ΔCₜ values in qRT-PCR data, log₂ transformed microarray data was used in the analysis, and the expression values of UBC minus those of selected probes were used in the normalization of the microarray data.

Tissue Microarrays (TMA). Samples from 2 retrospective collections of lung cancer were examined in TMA format from Yale University Pathology Archives; Cohort A (YTMA 250 [n=298]) and Cohort B (YTMA 79 [n=202]). TMAs consisted of 0.6 mm cores in 1 (Cohort A) and 2 fold (Cohort B) redundancy. TMAs were prepared according to standard methods. Cohort A comprises 314 serially collected NSCLC who underwent surgical resection of their primary tumor between 2004 and 2011. Cohort B comprises of 202 serially collected NSCLC patients who underwent surgical resection of their primary tumor between 1988 and 2003. All tissue was used after approval from the Yale Human Investigation Committee protocol #9505008219, which approved the patient consent forms or in some cases waiver of consent. The actual number of samples analyzed for each study is lower, due to unavoidable loss of tissue or the absence or limited tumor cells in some spots as is commonly seen in TMA studies. NSCLC patients in stage I, II, and IIIA were included in the analysis. Those who died with no evidence of disease were excluded from further analysis.

Quantitative immunofluorescence. FFPE whole-tissue sections, tissue microarrays (TMAs) and cell pellets were processed at Yale Cancer Center/Pathology Tissue Microarray Facility.

FFPE whole-tissue sections, tissue microarrays (TMAs) and cell pellets were processed as follows: briefly, sections were baked for 30 minutes at 60 degrees Centigrade and underwent two twenty minute wash cysles in xylenes. Slides were rehydrated in two 1-minute washes in 100% ethanol followed by one washing 70% ethanol and finally rinsed in streaming tap water for 5 minutes. Antigen retrieval was performed in sodium citrate buffer pH.6, for 20 minutes at 97 degrees Centigrade in a PT module (LabVision). Endogenous peroxidases were blocked by 30-minute incubation in 2.5% hydrogen peroxide in methanol. Nonspecific antigens were blocked by a 30 minute incubation in 0.3% BSA in TBST. Slides were then incubated with the target primary antibody (ZNF71 Abcam; ab87250), as well as pan cytokeratin (AE1/AE3) overnight at 4 degrees Centigrade diluted at 1:100 to define the tumor compartment.

Primary antibodies were followed by incubation with Alexa 546-conjugated goat anti-mouse secondary antibody (Life Technologies) diluted 1:100 in rabbit EnVision reagent (Dako) for 1 hour. ZNF71 signal was amplified with Cy5-Tyramide (Perkin Elmer) for 10 minutes, and then nuclei were stained with 0.05 mg DAPI in BSA-tween for 10 minutes. Slides were mounted with ProlongGold (Life Technologies). Two TBS-T and one TBS wash was performed between each step after the primary antibody.

Immunofluorescence was quantified using automated quantitative analysis (AQUA) Fluorescent images of DAPI, Cy3 (Alexa 546-cytokeratin), and Cy5 (ZNF71) for each TMA spot were collected. Image analysis was carried out using the AQUAnalysis software (Navigate Biopharma Inc.), which generated an AQUA score for each compartment by dividing the sum of target pixel intensities by the area of the compartment in which the target is measured. AQUA scores were normalized to the exposure time and bit depth at which the images were captured, allowing scores collected at different exposure times to be directly comparable. Specimens with less than 5% tumor area per region of interest were not included in AQUA analysis for not being representative of the corresponding tumor specimen.

Enzyme-Linked Immunosorbent Assay (ELISA). A total of 38 NSCLC patient tissue samples were selected for ELISA assays, including 29 tumor resections of lung adenocarcinoma and squamous cell lung cancer and 9 matched adjacent normal lung tissue samples. The DuoSet ELISA Development Systems from R&D Systems (Minneapolis, Minn.; catalog number: DY382-05) were used for quantifying protein expression of T-Cell Activation Antigen CD27 (CD27)/Tumor Necrosis Factor Receptor Superfamily, Member 7 (TNFRSF7) in NSCLC patient tissue samples, according to manufacturer's protocol. The ELISA assay results were quantified using the Synergy H1 Hybrid Multi-Mode Microplate Readers from BioTek Instruments, Inc. (Winooski, Vt.). Samples that yielded a positive OD values were included for further analysis. Statistical analysis was done using a two-sample t-test assuming unequal variances. The concordance between CD27 mRNA and protein expression was evaluated with Spearman correlation coefficient.

### Results:

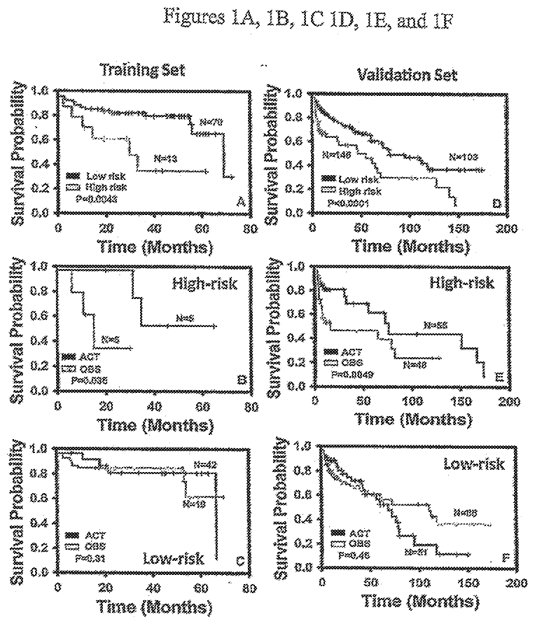

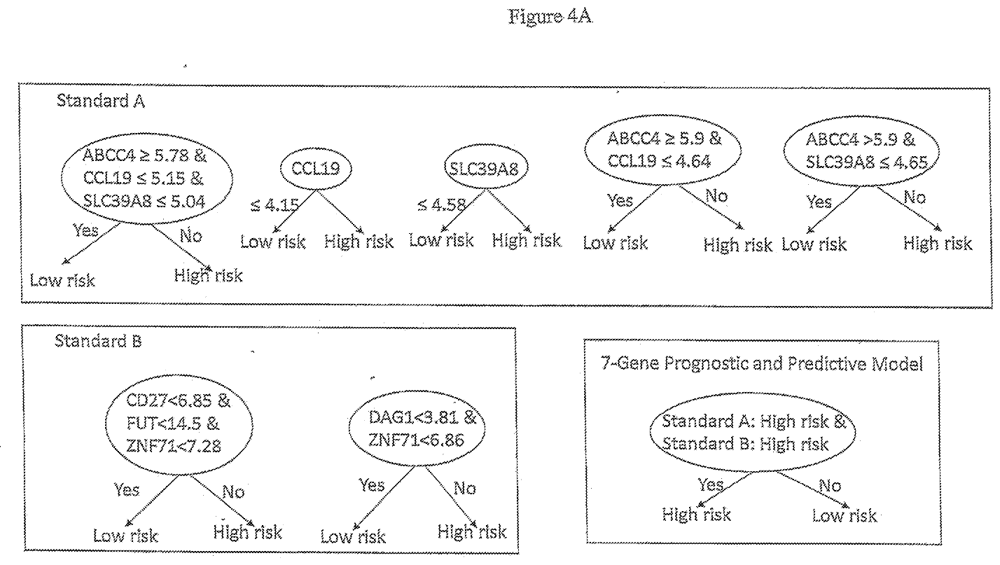

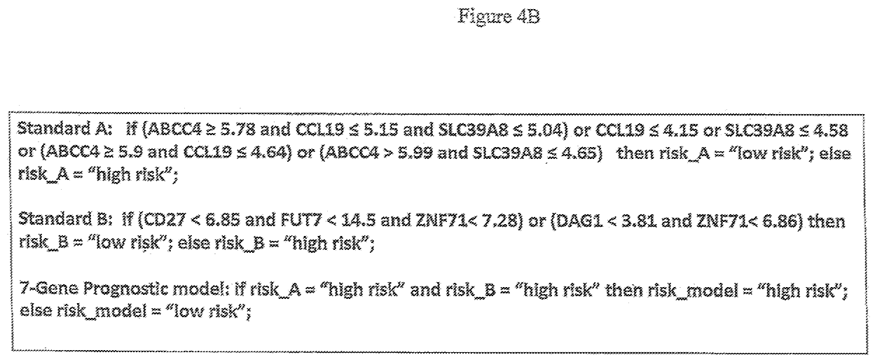

The NSCLC prognostic biomarkers identified with hybrid feature selection models [18, 19] and molecular network approach [20, 21] in our previous studies were validated with multiple independent microarray datasets. Based on the validation results in microarray data, 160 genes were selected for assays using low-density microfluidic qRT-PCR arrays. Among 160 genes analyzed in the qRT-PCR assays, a 7-gene signature of this invention was identified from training cohort obtained from Case Western Reserve University (CWRU; n=83). Details of the decision tree based 7-gene prognostic and predictive method of this invention are provided in FIG. 4A. In the training cohort (CWRU), the 7-gene model stratified patients into two prognostic groups with significantly different disease-specific survival (P=0.0043; FIG. 1A). Moreover, in the 7-gene assay predicted chemotherapy benefit (high-risk) patient group, there was a significant prolonged disease-specific survival (P=0.035; FIG. 1B) in adjuvant chemotherapy treated patients (ΔCT) compared with the observation group (OBS) who did not receive any chemotherapy. Specifically, the 30 months survival rate was less than 0.4 in the high-risk patients in who did not receive chemotherapy (the OBS group), and the 30 months survival rate was 100% (5/5) in patients receiving adjuvant chemotherapy (the ΔCT group). In contrast, there was no survival benefit in receiving chemotherapy (P=0.31; FIG. 1C) in the 7-gene assay predicted non-benefit (low-risk) group. Consistent prognostic and predictive results were confirmed in the validation set (n=248), including NSCLC patients from another three hospitals (UM, MBRCC, and NorthShore) as well as a clinical trial JBR.10 [14] (FIGS. 1D, 1E, and 1F). In the validation set, the 7-gene signature generated significant prognostic stratification (P<0.0001; FIG. 1D). In the predicted benefit (high-risk) patient group, there was a significant prolonged disease-specific survival in the ΔCT group compared with the OBS group (P=0.0049; FIG. 1E). Specifically, the 5-year survival rate was 70.9% (39/55) in the high-risk patients who received adjuvant chemotherapy (the ΔCT group), whereas the 5-year survival rate was 45.8% (22/48) in high-risk patients who did not receive adjuvant chemotherapy (the OBS group). In contrast, in the predicted non-benefit (low-risk) group, there was no survival benefit in the ΔCT group compared with the OBS group (P=0.46, FIG. 1F). It is noteworthy that in the predicted non-benefit (low-risk) group, patients who received adjuvant chemotherapy (ΔCT) had a worse post-surgical survival in the long term compared with those who did not receive any chemotherapy (OBS) in both training and validation sets (FIG. 1C and FIG. 1F). These results further corroborate the 7-gene model prediction of non-benefit that patients would suffer from unnecessary cytotoxicity side-effects of chemotherapy instead of benefiting from it. Overall, these results demonstrate that the 7-gene assay is both prognostic of NSCLC clinical outcome and predictive of the benefits from chemotherapy. In FIGS. 1B, 1C, 1E, and 1F the following abbreviations are used: ΔCT: Adjuvant chemotherapy group; OBS: observation group without chemotherapy. The validation set includes patient cohorts from MBRCC, UM, JBR.10, and Northshore. The 7-gene signature stratified patients into high-risk and low-risk groups in both training (FIG. 1A) and validation (FIG. 1D) sets. In the high-risk groups from training (FIG. 1B) and validation (FIG. 1E) sets, there were significant survival benefits in patients receiving adjuvant chemotherapy (the ΔCT group) compared with those who did not receive any chemotherapy (the OBS group). In the low-risk groups from FIG. 1C and validation FIG. 1F sets, there were no significant survival benefits in patients receiving adjuvant chemotherapy (the ΔCT group) compared with those who did not receive any chemotherapy (the OBS group). P values were assessed with log-rank tests.

The chemoresponse prediction for specific therapeutic agents was examined in the identified 7 biomarkers. In particular, gene expression of ATP binding cassette subfamily C member 4 (ABCC4) was predictive of chemoresistance in patients receiving carboplatin, cisplatin, and Taxol, with under-expressed mRNA (higher ΔCₜ) value associated with significantly decreased hazard ratio of death from disease and tumor recurrence (see Table 2). In patients treated with carboplatin plus Taxol, using ΔCₜ value of ABCC4 in Cox model, the hazard ratio of death from disease of was 0.43 (95% CI: [0.208, 0.888], P=0.02) and the hazard ratio of recurrence was 0.343 (95% CI: [0.122, 0.968], P=0.04), both statistically significant. In patients treated with Taxol, the hazard ratio of death from disease of ABCC4 ΔCₜ value was 0.403 (95% CI: [0.194, 0.834], P=0.01, Cox model) and the hazard ratio of recurrence was 0.48 (95% CI: [0.253, 0.912], P =0.02, Cox model), both statistically significant. In patients treated with either carboplatin plus Taxol, carboplatin plus Taxotere, cisplatin plus Taxotere, or cisplatin plus Taxol, the hazard ratio of death from disease of ABCC4 ΔCₜ values was borderline significant (hazard ratio: 0.528 [0.271, 1.028], P=0.06, Cox model) and the hazard ratio of recurrence was significant at 0.545 (95% CI: [0.298, 0.998], P=0.049, Cox model; Table 2). The expression of fucosyltransferase 7 (FUT7) was predictive of chemosensitivity to carboplatin, with under-expressed mRNA (higher ΔCₜ value) associated with significantly increased hazard ratio of death from disease (hazard ratio: 1.605 [1.058, 2.435], P=0.026, Cox model; Table 2). The expression of zinc finger protein 71 (ZNF7 I)(SEQ ID NO:9) was also predictive of chemosensitivity in patients treated with either carboplatin plus Taxol, carboplatin plus Taxotere, cisplatin plus Taxotere, or cisplatin plus Taxol, with a significant hazard ratio of death from disease 1.986 (95% CI: [1.001, 3.938], P=0.049, Cox model; Table 2). Solute carrier family 39 member 8 (SLC39A8) was predictive of chemoresistance to Taxol, with a borderline significant hazard ratio of recurrence 0.584 (95% CI: [0.33, 1.03], P=0.06, Cox model; Table 2). The expression of SLC39A8 was also predictive of chemoresistance to Alimta (pemetrexed), with a borderline significant hazard ratio of recurrence 0.49 (95% CI: [0.219, 1.098], P=0.08, Cox model; Table 2).

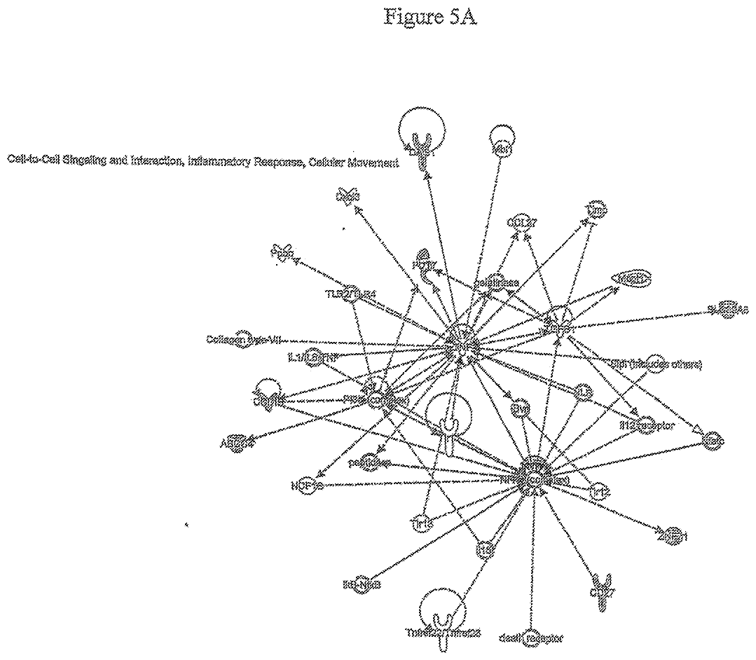

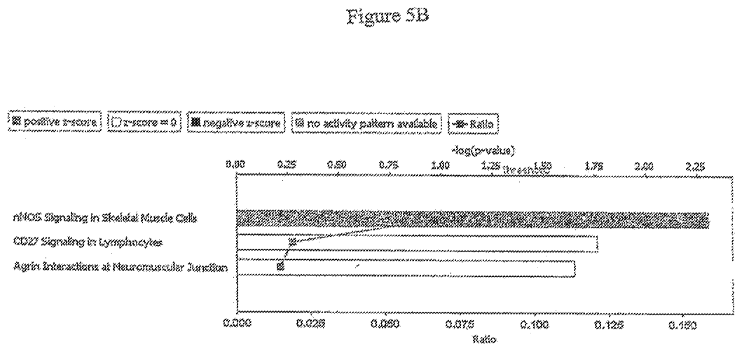

The 7-gene NSCLC prognostic and predictive signature is involved in cell to cell signaling and interaction, inflammatory response, and cellular movement in Ingenuity Pathway Analysis (Qiagen, Redwood City, Calif.). Based on the molecular network of the 7 NSCLC biomarkers (FIG. 5A), the identified biomarkers have interactions with major inflammatory and cancer signaling hallmarks such as TNF, PI3K, NF-κB, and TGF-β. The top pathways involving the 7 signature genes and their interaction partners are nNOS signaling in skeletal muscle cells, CD27 signaling in lymphocytes, and agrin interactions at neuromuscular junction (FIG. 5B). The 7-gene signature identified in this study does not overlap with the NSCLC gene signatures reported in previous studies [13, 15-17, 23-25].

Protein expression of ZNF71 (SEQ ID NO:9) is prognostic of NSCLC outcome.

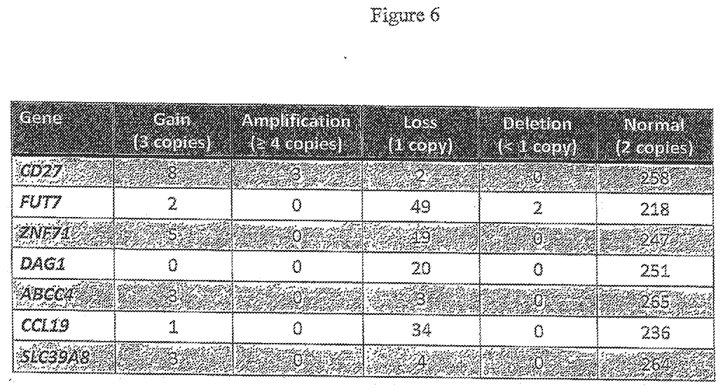

To substantiate the functional involvement of the identified 7 signature genes of the methods of this invention, protein expression of these biomarkers was evaluated with immunohistochemistry (IHC). Based on the IHC results, biomarkers with staining of good quality in FFPE NSCLC tumor tissues were further quantified with AQUA. Protein expression of ZNF71 (SEQ ID NO:9) was identified as prognostic of NSCLC outcome in two TMA cohorts (FIG. 5A). Based on the quantitative AQUA scores representing ZNF71 (SEQ ID NO:9) protein expression levels in tumor tissues, a cutoff point was defined for patient prognostic stratification in training cohort YTMA250 (n=145). Specifically, when logₑ-transformed ZNF71 (SEQ ID NO:9) AQUA scores were greater than or equal to 7.9, patients had significantly better disease-specific survival (P=0.021) than those with a lower ZNF71 (SEQ ID NO:9) protein expression level (FIG. 5B). This cutoff was further validated with significant patient stratification (P=0.047) in an independent cohort YTMA79 (n=46). Higher protein expression of ZNF71 (SEQ ID NO:9) is significantly associated with better patient survival, which is concordant with its mRNA results in multiple independent patient cohorts and its observed association with chemosensitivity in Taxol (Taxotere) plus platinum-based treatment in NSCLC patients (Table 2). These results indicate that ZNF71 (SEQ ID NO:9) is a prognostic protein biomarker and might be a potential therapeutic target of NSCLC. Furthermore, ZNF71 (SEQ ID NO:6) had a 7% (19/271) of loss of DNA copy number in a NSCLC patient cohort from Starczynowski et al [26] (n=271; FIG. 6). These results suggest the concordance in the loss of DNA copy number, down-regulated mRNA and protein expression of ZNF71 (SEQ ID NO:9) in lung cancer progression.

Concordant mRNA and protein under-expression in NSCLC progression:

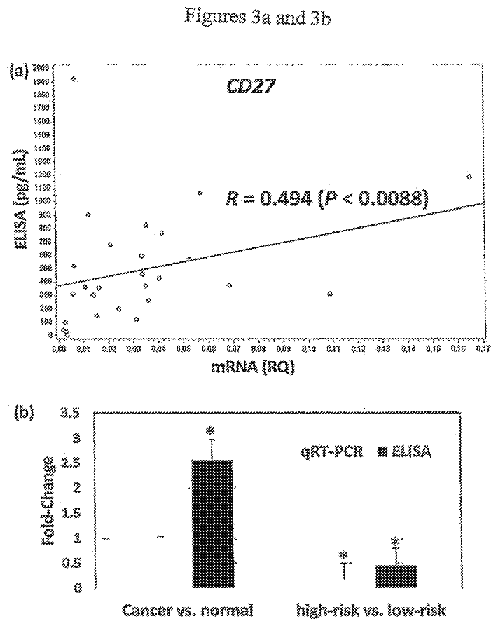

The protein expression level of CD27 (SEQ ID NO:10) was quantified with ELISA assays in FFPE NSCLC tumor tissues (n=29) and normal adjacent lung tissues (n=9). Spearman correlation coefficient between mRNA and protein expression of CD27 (SEQ ID NO:10) is 0.494 (P<0.0088; FIG. 3a) in tumor tissues. CD27 (SEQ ID NO:10) had an average protein expression of 599.06 pg/mL in low-risk patients with a better disease-specific survival, and an average protein expression of 245.5 pg/mL in high-risk patients with a poorer disease-specific survival in ELISA assays. CD27 (SEQ ID NO:10) had significant under-expression in high-risk patients vs. low-risk patients at mRNA level with a fold-change of 0.17 (P<0.00001) and a fold-change of 0.41 (P<0.02) at protein level (FIG. 3b). CD27 (SEQ ID NO:10) had an average protein expression of 191 pg/mL in normal lung tissues. CD27 (SEQ ID NO:10) had significant protein overexpression in NSCLC tumor vs. normal tissues with a fold-change of 2.56 (P<0.025), while mRNA expression in tumor vs. normal tissues was not significantly different (FIG. 3b). The over-expressed CD27 (SEQ ID NO:10) protein in NSCLC tumors is concordant with an observed 4% (11/271) of gain or amplification of DNA copy number in the NSCLC patient cohort from Starczynowski et al [26] (n=271; FIG. 6). Overall, these results demonstrate that CD27 (SEQ ID NO:10) had concordant under-expression at both mRNA and protein levels in NSCLC patients with a poor outcome and a greater chance of tumor recurrence and metastasis. The overexpressed CD27 (SEQ ID NO:10) protein level in FFPE NSCLC tumor vs. normal lung tissues indicates that CD27 regulation in tumorigenesis and metastatic processes is different. Our results confirm the role of CD27 (SEQ ID NO:10) as a target in lung cancer immunotherapy [27, 28].

Lung cancer is the second most common cancer in both men and women, and remains the highest cancer-related mortality with a death rate higher than colon, prostate, and breast cancer combined. Currently, there is no clinically available multi-gene assay to prognosticate and predict the benefits of chemotherapy in formalin fixed or paraffin embedded NSCLC tissues of NSCLC patients for improved personalized treatment. Immunotherapy is more effective and less toxic than chemotherapy in advanced lung cancers [5-8, 29, 30], and recent studies show promise of immunotherapy in early stage lung cancer patients [8]. Nevertheless, predictive biomarkers and therapeutic targets of immunotherapy are not well established.

There were abundant publically available microarray data generated in NSCLC patient tissues. Although microarray platforms are phasing out, the legacy data and biomarkers identified in microarray platforms are still useful in the RNA-seq era [9]. However, high-throughput platforms such as microarrays and RNA-seq are not suitable for routine clinical tests. Validation of biomarkers identified from high-throughput technologies with qRT-PCR emerges as the most promising experimental protocol for developing multi-gene assays for clinical applications.

NSCLC prognostic biomarkers were identified with hybrid feature selection models [18, 19, 31] and molecular network approach [20, 21] in our previous studies. The hybrid feature selection models [18, 19, 31] contain multiple layers of gene selection algorithms in the process of biomarker identification. This scheme takes advantage of different algorithms in different stages of gene shaving, in order to identify the gene signatures with the optimal performance. The molecular network approach [20, 21] constructs genome-scale co-expression networks in good-prognosis and poor-prognosis patient groups separately, and compares the network structures of these two patient groups to identify disease-specific network modules. Next, genes with concurrent co-expression with multiple major lung cancer signaling hallmarks were pinpointed from disease-specific network modules for further gene signature identification. This approach embedded biological relevance into biomarker identification. The signature genes identified with these sophisticated approaches were validated with multiple independent publically available microarray datasets. Genes with consistent expression patterns in multiple validation sets were included in qRT-PCR assays. The 7-gene signature of the methods of this invention identified in qRT-PCR assays was prognostic and predictive of chemoresponse in patient cohorts from multiple hospitals and JBR.10.

The identified 7 signature genes have interactions with major inflammatory and cancer signaling hallmarks including TNF, PI3K, NF-κB, and TGF-β (FIG. 5A). Multiple signature genes are potential targets in cancer immunotherapy. Specifically, reduction of DAG/may increase susceptibility of muscle fibers to necrosis [32]. A study shows that DAG-1 cells are resistant to TNF-α and IFNγ-induced apoptosis, with implications in bladder cancer progression and resistance to immunotherapy [33]. CD27 is part of TNF receptor family, and overexpression of CD27 induces NF-κB activation involving signaling transduction of TNF receptor-associated factors [34]. CD27 was also reported as a potential target of cancer immunotherapy [27, 28]. The synergy between PD-1 blockade and CD27 stimulation for CD8+ T-cell driven anti-tumor immunity was reported recently [35], indicating the therapeutic potential of CD27 in neoadjuvant PD-1 blockade in resectable lung cancer. The zinc finger protein ZNF71 is induced by TNF-α [37] and ZNF71 SNP was found to be associated with asthma in human serum [38]. CCL19 is regulated by multiple NF-κB and INF family transcription factors in human monocyte-derived dendritic cells [39]. ABCC4 is associated with multiple drug resistance in cancer [40] and smooth muscle cell proliferation [41], and interacts with PI3K in cancer prognosis and drug resistance [42]. Our results on ABCC4 in Table 2 are consistent with its functional role and reported drug resistance. FUT7 interacts with TNF-α in human bronchial mucosa [43] and its induction at sites of tumor cell arrest is involved in metastasis [44]. NF-κB was reported to regulate expression of the zinc transporter SLC39A8 [45]. Indirect interactions between TGF-β and SLC39A8 are involved in tumorigenesis [46] and fibrogenic response [47].

The 7-gene signature identified in the methods of this invention does not overlap with the NSCLC gene signatures reported in recent studies [15, 16, 23-25]. However, several biomarker genes identified in this study belong to the same families or functional categories as the biomarkers identified in [14-16]. In particular, FUT7 from the current study and FUT3 from Kratz et al [16] are both fucosyltransferase and involved in metabolism. In the 12-gene prognostic and predictive signature from Tang et al [15], two genes belong to the same family or share similar functions as the 7-gene signature. Specifically, SLC35A5 from Tang et al [15] and SLC39A8 from this study both belong to solute carrier superfamily, and ATPase Phospholipid Transporting 8A1 (ATP8A1) from Tang et al [15] and ATP Binding Cassette Subfamily C Member 4 (ABCC4) from this study are both involved in energy metabolism. The 15-gene prognostic and predictive gene signature of JBR.10 [14] also contains two genes that share similar functions as the 7-gene signature. ATPase Na+/K+ Transporting Subunit Beta 1 (ATP8A1) from Zhu et al [14] and ABCC4 from this study are again involved in energy metabolism, and ZNF236 from Zhu et al [14] and ZNF71 identified in this study both belong to zinc finger protein family. Overall, the 7-gene signature presented in this invention and two previous gene signatures from Zhu et al [14] and Tang et al [15] are all prognostic of NSCLC outcome and predictive of the benefits of chemotherapy. These three gene signatures all contain a biomarker related to ATP activities and energy metabolism. Other shared gene families between the 7-gene signature of this invention and these two signatures include zinc finger protein and solute carrier superfamily. The 7-gene signature and the practical prognostic gene assay for non-squamous NSCLC by Kratz et al [16] both contain biomarkers from fucosyltransferase family. These common gene families shared by the NSCLC gene signatures with promise for clinical utility might be functionally involved in tumor metastasis with implications in lung cancer therapy.

The protein expression of the identified 7 signature genes was also validated in this study. In particular, ZNF71 protein expression quantified with AQUA was a prognostic biomarker in two NSCLC patient cohorts (n=191). Higher mRNA and protein expression of ZNF71 is both associated with good prognosis, and ZNF71 mRNA is predictive of chemosensitivity in Taxol (paclitaxel) plus platinum-based treatment in NSCLC patients, and docetaxel plus platinum-based treatment in NSCLC patients. These results demonstrate that ZNF71 mRNA and protein expression can both be used in prognostication of NSCLC in clinical applications and ZNF71 may be a therapeutic target. CD27 had highly correlated mRNA and protein expression, with significant under-expression in poor prognostic (high-risk) NSCLC patients. CD27 mRNA and protein expression could potentially be used as a biomarker and target in lung cancer immunotherapy. Protein expression of CCL19 was also confirmed with ELISA in NSCLC tumor and adjacent normal tissues. CCL19 protein was under-expressed in FFPE NSCLC tumor tissues compared with normal lung tissues, with no statistically significant difference (results not shown). CCL19 also had lower protein expression in poor-prognosis (high-risk) NSCLC patients compared with good-prognosis (low-risk) patients, with no statistically significant difference (results not shown). The trend of CCL19 protein expression was qualitatively concordant with its mRNA expression that higher expression of CCL19 is associated with good prognostic outcome of NSCLC. CCL19 had a 12.5% (34/271) of a loss of DNA copy number in the NSCLC patient cohort from Starczynowski et al [26] (n=271; FIG. 6), which suggests a loss of DNA copy number and down-regulated mRNA and protein expression of CCL19 in NSCLC progression. In our previous integrated DNA copy number and gene expression regulatory network analysis of NSCLC metastasis, CCL19 is a driver gene and CD27 expression is modulated by CCL19 in squamous cell lung cancer patients with good prognosis [48]. Together with the molecular network reported in the literature (and see FIG. 5A), while not being bound to any particular theory, the interaction between CCL19 and CD27 could be through PI3K and NF-κB complexes. In addition, FUT7 and DAG1 had concordant loss or deletion of DNA copy number (FIG. 6) and down-regulated gene expression in NSCLC progression (Table 2 and FIG. 4A).

This invention provides a method of measuring the expression gene expression levels comprising determining the level of expression of the following multi-gene set consisting of ABCC4 (SEQ ID NO:1), CCL19 (SEQ ID NO:2), SLC39A8 (SEQ ID NO:3), CD27 (SEQ ID NO:4), FUT7 (SEQ ID NO:5), ZNF71 (SEQ ID NO:6), and DAG1 (SEQ ID NO:7). This method using this particular seven gene combination has never before been known to aid in the benefit of survival rates of patients afflicted with non-small cell lung cancer.

The method comprises the following steps: (1) extraction of total RNA from a formalin fixed or paraffin embedded tumor of non-small cell lung cancer after the surgical resection, (2) generation of complementary DNA (cDNA) of the extracted total RNA from a patient tumor, (3) quantification of mRNA expression of 7 genes: ABCC4 (SEQ ID NO:1), CCL19 (SEQ ID NO:2), SLC39A8 (SEQ ID NO:3) CD27 (SEQ ID NO:4), FUT7 (SEQ ID NO:5), ZNF71 (SEQ ID NO:6), and DAG1 (SEQ ID NO:7), (4) normalization of the quantification of the 7 genes with the quantification of a control gene UBC (SEQ ID NO:8), and (5) utilization of the normalized 7 gene mRNA expression quantification to predict whether a patient will benefit from receiving adjuvant chemotherapy or not. This method further comprises the step of predicting clinical benefit (i.e. prolonged disease-specific survival) of receiving adjuvant chemotherapy, including therapies selected from cisplatin and Taxol (paclitaxel), cisplatin and Taxotere (docetaxel), carboplatin, carboplatin and Taxol (paclitaxel), carboplatin and Taxotere (docetaxel), Taxol (paclitaxel), and Alimta (pemetrexed).

A preferred embodiment of this method includes use of a composition of only the following three: ABCC4 (SEQ ID NO:1), CCL19 (SEQ ID NO:2), and SLC39A8 (SEQ ID NO:3), within the 7-gene assays from this method, which also predicts the clinical benefit of receiving adjuvant chemotherapy. In another preferred embodiment of this method, the method includes use of a composition of only the following four genes: CD27 (SEQ ID NO:4), FUT7 (SEQ ID NO:5), ZNF71 (SEQ ID NO:6) and DAG1 (SEQ ID NO:7), within the 7-gene assays from the method, which also predicts the clinical benefit of receiving adjuvant chemotherapy.

Another method of this invention provides for the high expression of ABCC4 (SEQ ID NO:1) predicted chemoresistance to carboplatin and Taxol (paclitaxel), Taxol (paclitaxel), carboplatin and Taxotere (docetaxel), cisplatin and Taxotere (docetaxel), and cisplatin and Taxol (paclitaxel).

Another method of this invention provides for the high expression of FUT7 (SEQ ID NO:5) predicted chemosensitivity to carboplatin.

Another method of this invention provides for the high expression of ZNF71 (SEQ ID NO:6) predicted chemosentivity to carboplatin and Taxol (paclitaxel), carboplatin and Taxotere (docetaxel), cisplatin and Taxotere (docetaxol), and cisplatin and Taxol (paclitaxel).

Another method of this invention provides for the high expression of SLC39A8 (SEQ ID NO:3) predicted chemoresistance to Taxol (paclitaxel), and Alimta (pemetrexed).

Another method of this invention provides for the protein expression of ZNF71 (SEQ ID NO:9) quantified with automated quantitative analysis (AQUA) correlated with its mRNA expression in patient tumors. The protein expression of ZNF71 (SEQ ID NO:9) can independently classify patients into prognosis (longer survival) group or poor prognosis (shorter survival) group.

Another method of this invention provides for the protein expression of CD27 (SEQ ID NO:10) quantified with ELISA had a significant correlation with its mRNA in patient tumors and adjacent normal lung tissues, and could be an independent protein biomarker for patient prognosis and treatment selection.

TABLE 1
Clinical information of non-small cell lung cancer patient cohorts
collected for the qRT-PCR analysis.

| | | CWRU | MBRCC | UM | NorthShore | |
|---|---|---|---|---|---|---|
| | | (n = 89) | (n = 49) | (n = 101) | (n = 65) | |
| Age | Mean (Std error) | 70.11 | 66.70 | 67.04 | 69.64 (1.02) | |
| | | (0.94) | (1.25) | (0.96) | | |
| | <60 | 15 | 7 | 28 | 7 (10.77%) | |
| | | (15.15%) | (14.29%) | (27.72%) | | |
| | ≥60 | 84 | 39 | 73 | 48 (73.85%) | |
| | | (84.85%) | (79.59%) | (72.28%) | | |
| | Missing | | 3 (6.12%) | | 10 (15.38%) | |
| Sex | F | 52 | 23 | 53 | 34 (52.31%) | |
| | | (52.53%) | (46.94%) | (52.48%) | | |
| | M | 47 | 26 | 48 | 21 (32.31%) | |
| | | (47.47%) | (53.06%) | (47.52%) | | |
| | Missing | | | | 10 (15.38%) | |
| Smoking | Current | 43 | 1 (2.04%) | | Yes | 60 |
| | | (43.43%) | | | | (92.31%) |
| | Former | 40 | 3 (6.12 | | | |
| | | (40.40%) | %) | | | |
| | Never | 8 (8.08%) | | | No | 5 |
| | Passive | 1 (1.01%) | | | | (7.69%) |

| | Other | 1 (1.01%) | | | |
|---|---|---|---|---|---|
| | Missing | 6 (6.06%) | 45 | | |
| | | | (91.48%) | | |
| AJCC stage | I | 46 | 27 | 59 | 46 (70.77%) |
| | | (46.46%) | (55.10%) | (58.42%) | |
| | II | 46 | 16 | 16 | 15 (23.08%) |
| | | (46.46%) | (32.65%) | (15.84%) | |
| | III | 6 (6.06%) | 6 | 26 | 4 (6.15%) |
| | | | (12.25%) | (25.74%) | |
| | Missing | 1 (1.01%) | | | |
| Chemotherapy | Yes | 29 | 27 | 24 | 28 (40.03%) |
| | | (29.29%) | (55.10%) | (23.76%) | |
| | No | 52 | 20 | 77 | 36 (55.38%) |
| | | (52.53%) | (40.82%) | (76.24%) | |
| | Missing | 13 | 2 (4.08%) | | 1 (1.54%) |
| | | (13.13%) | | | |
| Histology | Adenocarcinoma | 65 | 27 | 101 | 43 (66.15%) |
| | | (65.66%) | (55.10%) | (100%) | |
| | Squamous | 27 | 14 | | 11 (16.92%) |
| | | (27.27%) | (28.57%) | | |
| | Other | 7 (7.07%) | 8 | | 6 (9.23%) |
| | | | (16.33%) | | |
| | Missing | 5 (5.05%) | | | 5 (7.69%) |
| Differentiation | Well | 5 (5.05%) | | 28 | 20 (30.77%) |
| | | | | (27.72%) | |
| | Moderate | 44 | | | 4 (6.15%) |
| | | (44.44%) | | | |
| | Moderate to | 4 (4.04%) | | 39 | 22 (33.85%) |
| | Poorly | | | (38.61%) | |
| | Poorly | 35 | | 34 | 17 (26.15%) |
| | | (35.35%) | | (33.66%) | |
| | Missing | 11 | | | 2 (3.08%) |
| | | (11.11%) | | | |
| Tumor Grade | 1 | 5 (5.05%) | 3 (6.12%) | | 20 (30.77%) |
| | 2 | 44 | 18 | | 19 (29.23%) |
| | | (44.44%) | (36.73%) | | |
| | 3 | 36 | 22 | | 21 (32.31%) |
| | | (36.36%) | (4.90%) | | |
| | Other | 3 (3.03%) | | | |
| | Missing | 11 | 6 | | 5 (7.69%) |
| | | (11.11%) | (12.25%) | | |

TABLE 2
Predictive biomarkers of chemoresponse in non-small cell lung cancer.
Hazard ratios were computed with Cox proportional hazard model using
ΔCₜ values in qRT-PCR assays.

| | | Hazard ratio | Hazard ratio | |
|---|---|---|---|---|
| | | of death | of | |
| | | from | recurrence | |
| | Chemotherapeutic | disease with | with 95% | Chemosensitive/ |
| Genes | agents | 95% CI | CI | resistant |
| ABCC4 | Carboplatin + | 0.43 [0.208, | 0.343 | Chemoresistant |
| SEQ ID | Taxol | 0.888]* | [0.122, | |
| NO: 1 | | | 0.968]* | |
| | Taxol | 0.403 | 0.48 [0.253, | Chemoresistant |
| | | [0.194, | 0.912]* | |
| | | 0.834]* | | |
| | Carboplatin + | 0.528 | 0.545 | Chemoresistant |
| | Taxol | [0.271, | [0.298, | |
| | Carboplatin + | 1.028]^{#} | 0.998]* | |
| | Taxotere | | | |
| | Cisplatin + | | | |
| | Taxotere | | | |
| | Cisplatin + Taxol | | | |
| FUT7 | Carboplatin | 1.605 | — | Chemosensitive |
| SEQ ID | | [1.058, | | |
| NO: 5 | | 2.435]* | | |
| ZNF71 | Carboplatin + | 1.986 | | Chemosensitive |
| SEQ ID | Taxol | [1.001, | | |
| NO: 6 | Carboplatin + | 3.938]* | | |
| | Taxotere | | | |
| | Cisplatin + | | | |
| | Taxotere | | | |
| | Cisplatin + Taxol | | | |
| SLC39A8 | Taxol | — | 0.584 [0.33, | Chemoresistant |
| SEQ ID | | | 1.03]^{#} | |
| NO: 3 | Alimta | — | 0.49 [0.219, | Chemoresistant |
| | | | 1.098]^{#} | |

*Hazard ratio significant at p <0.05
^{#}Hazard ratio borderline significant at p <0.08

This invention presents a method using a 7-gene predictive assay based on qRT-PCR to improve NSCLC treatment in clinics using formalin fixed or paraffin embedded samples. This method using a 7-gene assay provides accurate prognostication and prediction of the clinical benefits of chemotherapy in multiple patient cohorts from the US hospitals and the clinical trial JBR.10. The 7-gene assay is enriched in inflammatory response. The protein expression of ZNF71 (SEQ ID NO:9) is prognostic of NSCLC outcome in two independent patient cohorts, which is concordant with its mRNA expression. These results demonstrate that ZNF71 (SEQ ID NO:9) is a prognostic protein biomarker and a useful therapeutic target of NSCLC. The protein expression of CD27 (SEQ ID NO:10) was strongly correlated with its mRNA expression in NSCLC tumor tissues, and serves as a biomarker and target of immunotherapy in lung cancer. Multiple signature genes had concordant DNA copy number variation, mRNA and protein expression in NSCLC progression. The results presented in this invention are important for precision therapy in NSCLC patients, and further provides implications in developing new therapeutic strategies to combat this deadly disease.

This invention provides a method of treating a patient using a 7-gene assay that is predictive of clinical benefits of a patient receiving Alimta (pemetrexed for injection) and commercially available from Eli Lilly and Company, Indianapolis, Ind., USA. Alimta® product is a chemotherapy for the treatment of advanced nonsquamous non-small cell lung cancer (NSCLC). Alimta® is a registered trademark owned or licensed by Eli Lilly and Company.

This invention provides for the protein expression of ZNF71 (SEQ ID NO:9) that is a prognostic marker of non-small cell lung cancer. This invention provides a method of using the expression of ZNF71(SEQ ID NO:9) quantified with AQUA (i.e. Automated Quantitative Analysis ((AQUA)) of In Situ Protein Expression, to identify which patients having non-small cell lung cancer are likely to have good prognosis, and which patients are likely to be poor prognosis.

This invention provides an aid to help physicians determine which non-small cell lung cancer patients, who were initially treated with surgery, will benefit from chemotherapy or immunotherapy. The seven gene assay of the methods of this invention is an aid to predict which patients would benefit from chemotherapty and had significantly prolonged survival time compared to those patients who did not receive any chemotherapy, and which patients would not benefit from chemotherapy and whose long-term post surgical survival time was shorter compared to patients who also had surgery but did not receive any chemotherapy.

This invention provides a method for treating a patient having NSCLC comprising identifying two genes, CD27 (SEQ ID NO:4) and ZNF71 (SEQ ID NO:6), as useful in predicting patient outcomes and developing therapeutic targets in non-small cell lung cancer treatment.

It will be understood by those persons skilled in the art that this invention provides a multi-gene combination assay that provides guidance on the clinical benefits of providing chemotherapy to an individual having non-small cell lung cancer. This invention provides a method for providing precision medicine for lung cancer patients and provides therapeutic targets in both chemotherapy and immunotherapy.

This invention provides a method for improving personalized treatment of individuals having non-small cell lung cancer. Specifically, this invention provides a RT-PCR based method using a 7 gene assay for providing clinical benefits of chemotherapy to a patient having non-small cell lung cancer. This invention provides a prognostic protein biomarker ZNF71(SEQ ID NO:9) using AQUA technique. This invention provides a prognostic mRNA and protein biomarker CD27 (SEQ ID NO:10) with use in immunotherapy. This invention aids patients having non-small cell lung cancer who may benefit from chemotherapy. The protein biomarkers of this invention are new therapeutic targets in chemotherapy and immunotherapy.

IHC results on ZNF71 and CD27 in non-small cell lung cancer (NSCLC) formalin fixed paraffin embedded (FFPE) samples:

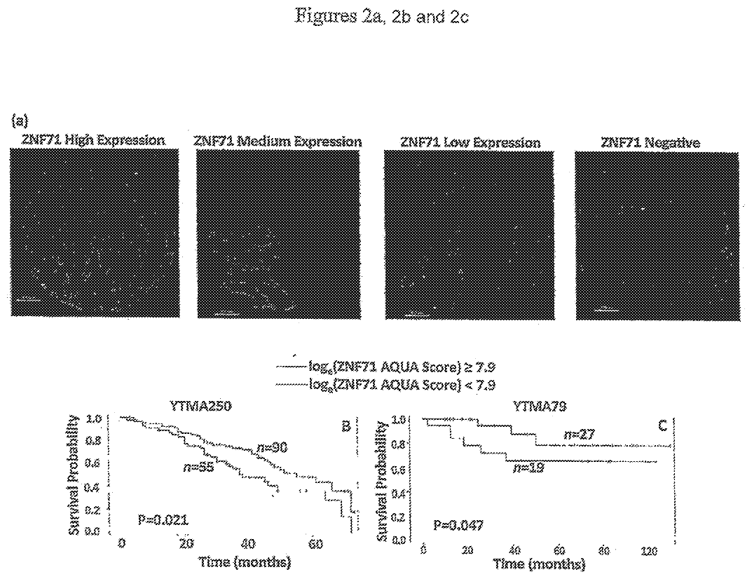

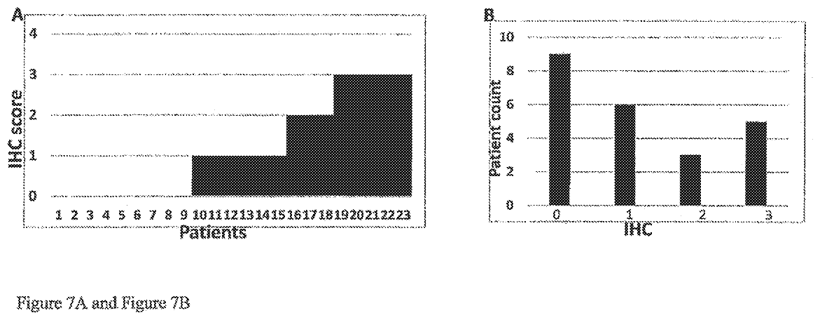

The immunohistochemistry assay was performed on ZNF71 and CD27 at Translational Pathology Research Laboratory at West Virginia University. A certified pathologist generated a score for the IHC staining in the following range: 0, 1, 2, 3, and 4, with 0 representing no staining and 4 the maximum staining. FIGS. 7A and 7B show the results of ZNF71 in NSCLC FFPE samples (n=24). It will be appreciated that the results in FIG. 2, previously presented in PCT Application serial No. PCT/US2019/036953, were generated using immunofluorescence on FFPE samples and the protein expression in these patient samples was quantified with Automated Quantitative Analysis (AQUA) at Yale Pathology Laboratory. Since immunofluorescence is not commonly available in most hospitals, the present invention, discloses a new clinical test using IHC for wider clinical applicability. CD27 stained the lymphocytes in the background, but did not generate any staining in the tumors. Since CD27 is involved in immune function in T cells and B cells, we use immunocytochemical staining of CD27 in T and B lymphocytes using protocols published in Ghosh, Spriggs [52].

The present invention provides a method of providing a treatment to a patient having non-small cell lung cancer comprising extracting total RNA from a formalin fixed and paraffin embedded tumor of a non-small cell lung cancer of a patient after the surgical resection; generating complementary DNA (cDNA) of the extracted total RNA from said formalin fixed or paraffin embedded patient tumor; quantifying of mRNA expression of 7 genes of ABCC4 (SEQ ID NO:1), CCL19 (SEQ ID NO:2), SLC39A8 (SEQ ID NO:3), CD27 (SEQ ID NO:4), FUT7 (SEQ ID NO:5), ZNF71 (SEQ ID NO:6); and DAG1 (SEQ ID NO:7) using qRT-PCR; normalizing of the quantification of said 7 genes with the quantification of a control gene UBC (SEQ ID NO:8) or a housekeeping gene; and utilizing said normalized 7 gene mRNA expression quantification to determine whether said patient will benefit from receiving adjuvant chemotherapy or not. This method further comprises administering to said patient a therapeutically effective amount of one of the following adjuvant chemotherapies (a) cisplatin and Taxol (paclitaxel), (b) cisplatin and Taxotere (docetaxel), (c) carboplatin, (d) carboplatin and Taxol (paclitaxel), (e) carboplatin and Taxotere (docetaxel), (f) Taxol (paclitaxel), and (g) Alimta (pemetrexed). In this method, the quantification of mRNA expression of three genes of ABCC4 (SEQ ID NO:1), CCL19 (SEQ ID NO:2), and SLC39A8 (SEQ ID NO:3) within said 7-genes. This method includes wherein said quantification of mRNA expression of four genes of CD27 (SEQ ID NO:4), FUT7 (SEQ ID NO:5), ZNF71 (SEQ ID NO:6), and DAG1 (SEQ ID NO:7) within said 7-genes. This method includes wherein said quantification of mRNA expression of ABCC4 (SEQ ID NO:1) and utilization of said normalized ABCC4 gene mRNA expression quantification to determine whether said patient will benefit from receiving adjuvant chemotherapy of one of (a) cisplatin and Taxol (paclitaxel), (b) cisplatin and Taxotere (docetaxel), (d) carboplatin and Taxol (paclitaxel), (e) carboplatin and Taxotere (docetaxel), and (f) Taxol (paclitaxel). This method includes wherein said quantification of mRNA expression of FUT7 (SEQ ID NO:5) and utilization of said normalized FUT7 gene mRNA expression quantification to determine whether said patient will benefit from receiving adjuvant chemotherapy of carboplatin. This method includes wherein said quantification of mRNA expression of ZNF71 (SEQ ID NO:6) and utilization of said normalized ZNF71 gene mRNA expression quantification to determine whether said patient will benefit from receiving adjuvant chemotherapy of (a) carboplatin and Taxol (paclitaxel), (b) carboplatin and Taxotere (docetaxel), (c) cisplatin and Taxotere (docetaxel), and (d) cisplatin and Taxol (paclitaxel). In another embodiment of this method, the method includes wherein said quantification of mRNA expression of SLC39A8 (SEQ ID NO:3) and utilization of said normalized SLC39A8 gene mRNA expression quantification to determine whether said patient will benefit from receiving adjuvant chemotherapy of one of (a) Taxol (paclitaxel), and (b) Alimta (pemetrexed).

In another embodiment of this invention, the method provides a treatment to a patient having non-small cell lung cancer comprising: providing protein expression of ZNF71 (SEQ ID NO: 9); quantifying said protein expression of said ZNF71 with automated quantitative analysis (AQUA) correlated with said ZNF71 mRNA expression in a formalin fixed and paraffin embedded patient tumor using immunohistochemistry staining; and determining a prognosis of said patient from said protein expression of said ZNF71. This method includes wherein said prognosis of said patient is either longer survival or shorter survival.

Another embodiment of this invention provides a treatment to a patient having non-small cell lung cancer comprising: providing protein expression of CD27 (SEQ ID NO:10); quantifying said protein expression of said CD27 with ELISA correlated with said CD27 mRNA expression in a formalin fixed and paraffin embedded patient tumor using immunocytochemistry staining and a cancer-free tissue adjacent to said tumor; and determining a prognosis of said patient from said protein expression of said CD27. This method includes wherein said prognosis of said patient is either longer survival or shorter survival. This method includes administering to said patient a therapeutically effective amount of an adjuvant chemotherapy.

FIGS. 7A and 7B show immunohistochemistry (IHC) staining results of ZNF71 in NSCLC patient FFPE samples (n=24). FIG. 7A shows ZNF71 IHC scores in the study cohort. FIG. 7B shows a summary of distribution of ZNF71 IHC scores in the study cohort.

### Summary of the Study on PersonalizedRx:

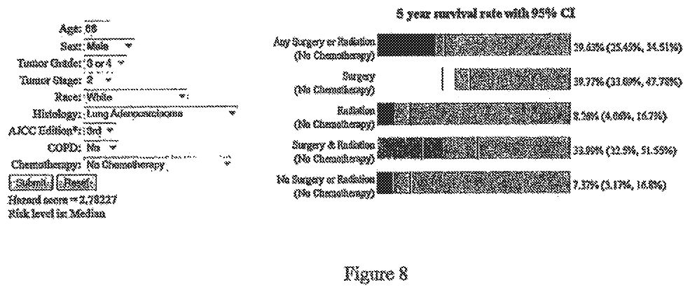

Data on 34,203 lung adenocarcinoma and 26,967 SQCLC patients in linked SEER-Medicare databases were used to determine the contribution of COPD, cancer stage, age, gender, race, and tumor grade to prognostication in 30 treatment combinations. A Cox model including these variables was estimated on 1,000 bootstrap samples, with the resulting model assessed on ROC, Brier Score, Harrell's C, and Nagelkerke's R² metrics. The comprehensive model was evaluated with two additional patient cohorts (n=1,994). The results show that combining patient information on COPD, cancer stage, age, gender, race, and tumor grade improves prognostication and prediction of treatment response in individual NSCLC patients receiving surgery, radiation, and chemotherapy, including Platinum-based, Platinum/Taxane, and Carboplatin/Paclitaxel/Avastin (Putila, Remick, and Guo 2011[51]; Putila and Guo 2014) [50]). An example of an online web-based model as a prognostic tool, is the model available at www.personalizedrx.org which has been used in the MBRCC clinic to advise patient treatment selection (see, FIG. 8).

FIG. 8 shows an example of output from the web-based model of the comprehensive prognostic model PersonalizedRx. Given the patient information submitted by the user (FIG. 8-left), the web-based tool will estimate survival for each treatment category using the survival observed for patients of a particular treatment modality and similar Hazard Score (FIG. 8-right).

Integration of IHC of ZNF71, immunocytochemical staining of CD27, qRT-PCR of 7 gene assay in FFPE samples with PersonalizedRx:

The present invention provides a method that provides a comprehensive prognostic model combining COPD, age, gender, race, histology, AJCC staging edition, cancer stage and tumor grade using multivariate Cox model with SEER-Medicare data (Putila, Remick, and Guo 2011 [51]; Putila and Guo 2014 [50]). All the model covariates are available in our clinical cohorts. Since all NSCLC patients receive pulmonary function tests before surgery, we will capture FEV1 and DLCO parameters to refine the diagnosis of COPD and its coefficient in the comprehensive prognostic model. The molecular biomarkers will be integrated with this model as independent covariate(s) with coefficients of other clinical covariates adjusted in the Cox model using training clinical cohort; the new model parameters will be validated with multiple independent clinical cohorts. The IHC scores of ZNF71 will be used as a co-variate in the multivariate Cox proportional hazard model used to contruct the PersonalizedRx tool (web-based model). Similar, the immunocytochemical staining results of CD27 in T cells and B cells will be used as co-variates in the above model, so will be the output from the qRT-PCR of the 7-gene assay in FFPE patient samples. We have published such analysis in previous studies (Wan et al. 2012 [54]). Table 3 is an example of the results from the analysis.

An interactive web interface of the current comprehensive web-based model available at http://www.personalizedrx.org has been used in clinics to aid treatment intervention. Validated molecular biomarkers will be added into this tool for improved cancer care.

This represents an additional prognostication improvement over the use of cancer stage alone, which has already been validated with statistical rigor [51]. This comprehensive model enables refined prognosis and estimation of clinical outcome of treatment combinations in NSCLC patients, providing a useful tool in personalized clinical decision-making. The comprehensive web-based model online tool commercially available at www.personalizedRx.org is employed herein the method of this invention and is in use at clinics at Mary Babb Randolph Cancer Center (MBRCC), West Virginia University. With the advancement of clinical genomics research, this comprehensive prognostic model is integrated with the genomic biomarkers to predict NSCLC patient treatment response in the methods of this invention.

TABLE 3
Multivariate Cox proportional analysis of the 7-gene risk score and major
clinical covariates in smoking lung cancer patients from the test cohort
(MSK and DFCI) in Director's Challenge Study (Shedden et al. 2008[53]).

| Variable* | P-value | Hazard Ratio (95% Cl)^{ψ} | |
|---|---|---|---|
| Analysis without 7-gene risk score | | | |
| Gender (Male) | 0.55 | 1.17 | (0.70, 1.95) |
| Age at diagnosis (>60) | 0.35 | 1.31 | (0.74, 2.29) |
| Tumor differentiation | | | |
| Moderately differentiated | 0.30 | 0.63 | (0.26, 1.51) |
| Poorly differentiated | 0.89 | 1.06 | (0.47, 2.38) |
| Cancer Stage | | | |
| Stage II | 1.54E−03 | 2.60 | (1.44, 4.71) |
| Stage III | 5.53E−05 | 4.48 | (2.16, 9.29) |
| Analysis with 7-gene risk score | | | |
| Gender (Male) | 0.51 | 1.19 | (0.71, 1.99) |
| Age at diagnosis (>60) | 0.49 | 1.22 | (0.69, 2.16) |
| Tumor differentiation | | | |
| Moderately differentiated | 0.33 | 0.65 | (0.27, 1.55) |
| Poorly differentiated | 0.93 | 0.96 | (0.43, 2.16) |
| Cancer Stage | | | |
| Stage II | 1.64E−03 | 2.61 | (1.44, 4.74) |
| Stage III | 3.29E−05 | 4.79 | (2.29, 10.04) |
| 7-gene risk score | 0.03 | 1.89 | (1.06, 3.38) |

*Gender was a binary variable (0 for female and 1 for male); age at diagnosis was a binary variable (0 for <60 years old and 1 otherwise); tumor grade was categorical variable of 3 categories (Well [as the reference group], Moderately, and Poorly differentiated); tumor stage was categorical variable of 3 categories (Stage I [as the reference group], Stage II, and Stage III).
^{ψ}denotes confidence interval.

### Supplementary Methods

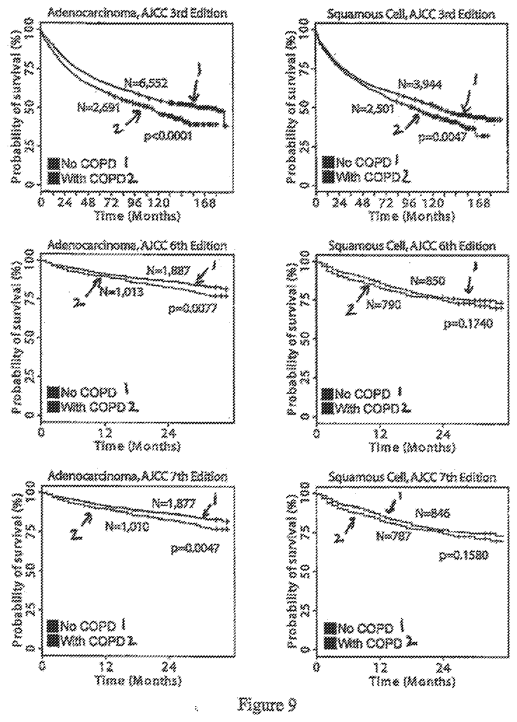

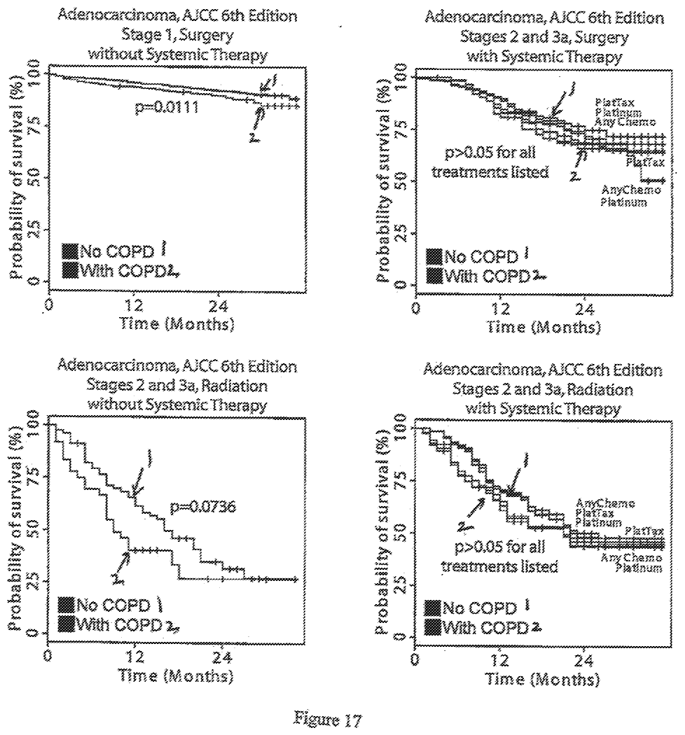

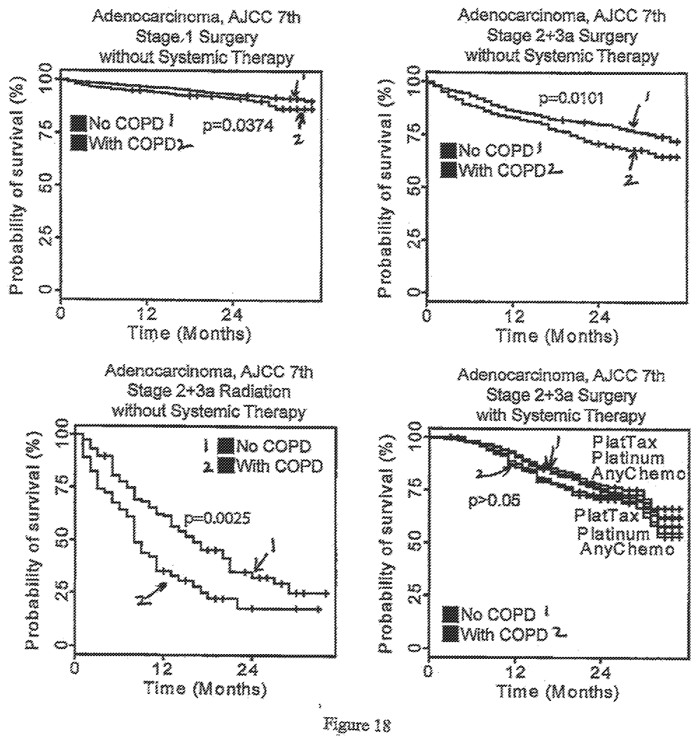

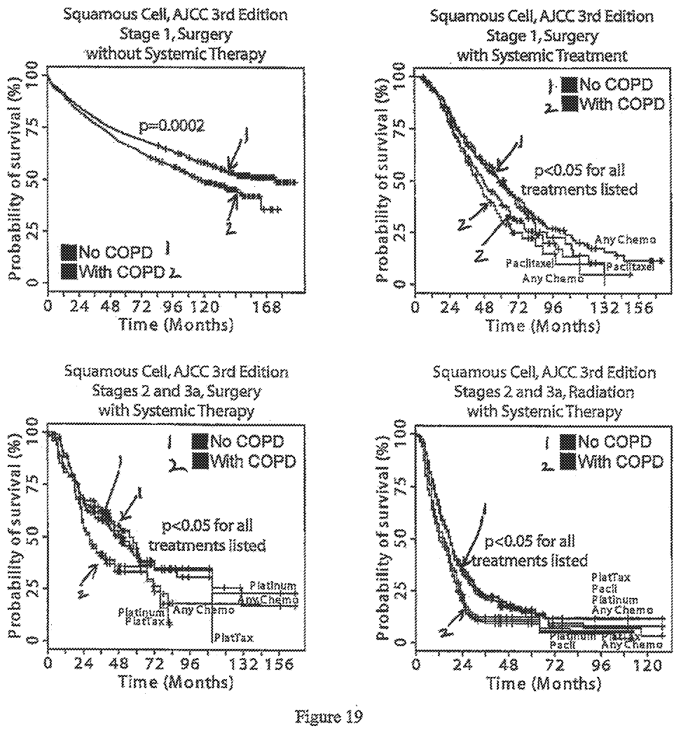

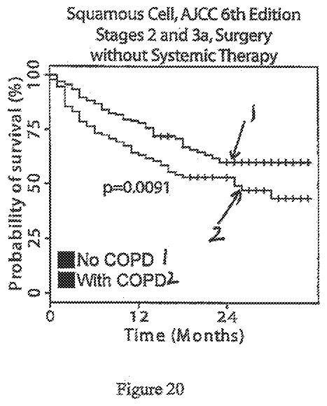

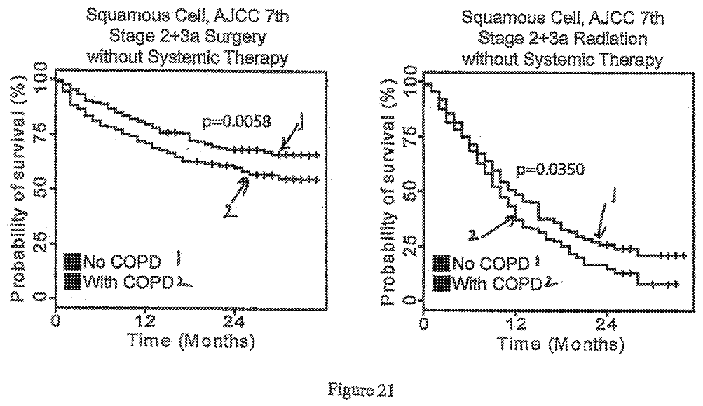

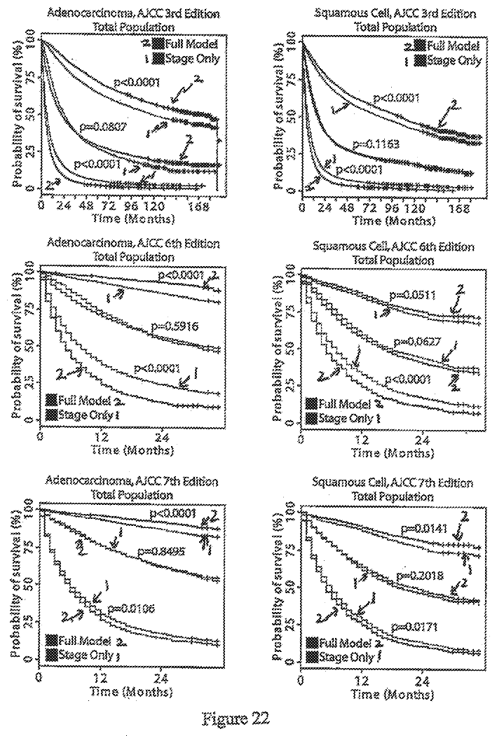

Each co-morbid condition was assessed as an independent predictor of survival using Cox proportional hazards modeling in patients treated with surgery but without chemotherapy or radiation indicated in order to isolate the effects of comorbidity from those of disparate treatment benefit or treatment candidacy (Supplementary Table 1). Additionally, the presence of COPD as determined via the analysis of administrative records was assessed as an independent predictor of survival by testing for significant stratification of Kaplan-Meier survival curves. Patients were split into two outcome groups based on COPD status, and separate survival curves were estimated and plotted (FIG. 9). Again, only patients receiving surgery without radiation or chemotherapy were included in order to better isolate the effect of COPD from other effects resulting from disparate treatment candidacy. The significance of the difference in survival was determined using the G-rho family of tests, with a p-value less than 0.05 indicating a significant difference in the survival curves estimated for the two groups being compared. As an additional test of the effect of COPD, patients were then further split into stage groups and the effect of COPD in these subsets was assessed for each group. A selection of these results can be seen in FIGS. 16-22. FIG. 16 shows the effect of COPD in Adenocarcinoma AJCC 3^{rd} Edition stage and treatment sub-groups. For each group, a clear and significant difference between the survival curves for patients with and without COPD can be seen, with patients identified as having COPD experiencing significantly poorer survival compared to those without COPD. FIG. 17 shows the effect of COPD in Adenocarcinoma AJCC 6^{th} Edition stage and treatment sub-groups. For each group, patients without COPD tend to experience longer survival when compared to patients with COPD, although the difference is not significant in some cases shown. FIG. 18 shows the effect of COPD in Adenocarcinoma AJCC 7^{th} Edition stage and treatment sub-groups. For each group treated without chemotherapy, a clear and significant difference between the survival curves for patients with and without COPD can be seen, with patients identified as having COPD experiencing significantly poorer survival compared to those without the disease. The difference in survival for patients treated with systemic therapy was not significant, but trended toward patients with COPD having poorer survival. FIG. 19 shows the effect of COPD in Squamous Cell AJCC 3^{rd} Edition stage and treatment sub-groups. For each group, a clear and significant difference between the survival curves for patients with and without COPD can be seen, with patients identified as having COPD experiencing significantly poorer survival compared to those without the disease. FIG. 20 shows the effect of COPD in Squamous Cell AJCC 6^{th} Edition stage and treatment sub-groups. For the group shown, a clear and significant difference between the survival curves for patients with and without COPD can be seen, with patients identified as having COPD experiencing significantly poorer survival compared to those without the disease. FIG. 21 shows the effect of COPD in Squamous Cell AJCC 7^{th} Edition stage and treatment sub-groups. For each group, a clear and significant difference between the survival curves for patients with and without COPD can be seen, with patients identified as having COPD experiencing significantly poorer survival compared to those without the disease.

FIG. 22 shows improvement in the Full model using COPD over Stage Alone. For each Kaplan-Meier plot, the three pairs of lines represent the High, Intermediate, and Low-Risk groups defined for each of the two models shown. The model using only the AJCC Stage is shown in orange color (1), while the Full model with COPD status added is shown in blue color (2). For each plot shown, the Full model with COPD status was able to produce a Low-Risk group with better survival and a High-Risk group with poorer survival, with most cases being significant (p<0.05).

The distributions of COPD and other variables in the original model were assessed in patients with very long and very short survival, relative to other patients, to determine if certain characteristics were disparate between groups of patients with varied survival. This was accomplished by partitioning patients based on survival time and status, then using a t-test or test of proportions to compare the distributions of variables between each group. Again, only those patients who were treated with surgery without radiation or chemotherapy were included. Long survival was defined as greater than 60 months for the original 3^{rd} Edition staging, and greater than 24 months for the 6^{th} and recoded 7^{th} Edition groups due to shortened follow-up. Short survival was defined as less than 24 months for the original 3^{rd} Edition staging, and less than 12 months for the 6^{th} and recoded 7^{th} Edition groups (Supplementary Table 3).

### Supplementary Results

COPD showed significant prognostic ability on multiple measures, both as an independent predictor and in the presence of other predictors. Other co-morbid conditions also showed promise as independent predictors in a Cox model (Supplementary Table 2). As an independent predictor, COPD status alone was able to significantly stratify patients into high and low-risk groups (p<0.05) in four of six groups (FIG. 9), although small sample size in the newer squamous cell carcinoma groups may have impeded achieving a significant stratification. The stratification in squamous cell carcinoma cases coded in the original 6^{th} Edition and those recoded to the 7^{th} Edition of AJCC staging was not significant despite a small degree of separation, with COPD patients having slightly diminished survival concordant with the other significant groups. In the significant cases, those without COPD showed consistently and significantly better survival when compared to those with COPD across the entire length of available follow-up, indicating that the effects of COPD are manifested in both long and short-term survival (FIG. 17).

The proportion of patients with COPD between those with relatively long and short survival was also assessed. Two survival cutoffs were used to split patients into three groups of short, intermediate, and long survival in order to test for differences in the distribution of each prognostic factor between groups of patients with relatively different survival. The difference in the prevalence of COPD between the short and long survival groups was assessed using a test of proportions. This test showed that COPD was much more prevalent in patients with relatively short survival when compared to those surviving relatively longer (Supplementary Table 4). This was true for each histology and coding scheme, despite differences in cutoffs and length of follow-up between the original and recoded staging systems. When the same test was performed for the other covariates similar results were seen, with factors previously seen to favor increased or diminished survival being disparate between the groups. These results are summarized in Supplementary Tables 4 through 9.

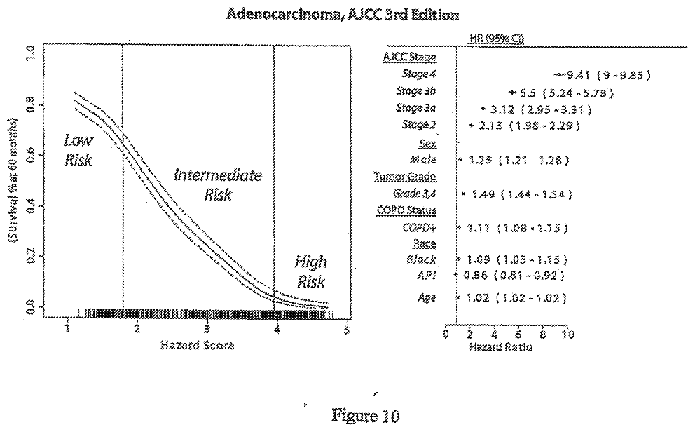

Patients were able to be split into high and low-risk groups with significantly different survival curves using COPD status alone in a variety of treatment and stage sub-groups. For adenocarcinoma patients staged using the original 3^{rd} Edition staging, there was a significant difference in the survival of Stage I patients treated with surgery alone. There was also a significant difference seen in Stage 2 and 3a patients treated with surgery with a platinum and taxane or without systemic therapy (p<0.05). A fourth stratification in Stage 2 and 3a patients treated with radiation and systemic therapy was also present (p<0.05, FIG. 10).

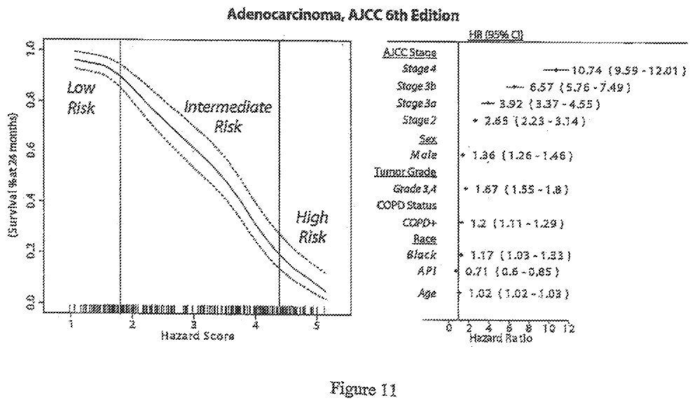

In the group of patients staged using the original 6^{th} Edition, Stage 2 and 3a radiation patients treated both with and without systemic therapy trended toward a similar stratification but did not achieve significance (p>0.05). A similar trend was observed for Stage 2 and 3a surgical patients treated with systemic therapy, although the degree of separation was again not significant (p>0.05). The group of stage 1 surgical patients treated without systemic therapy did however achieve a significant stratification (p=0.0111, FIG. 11).

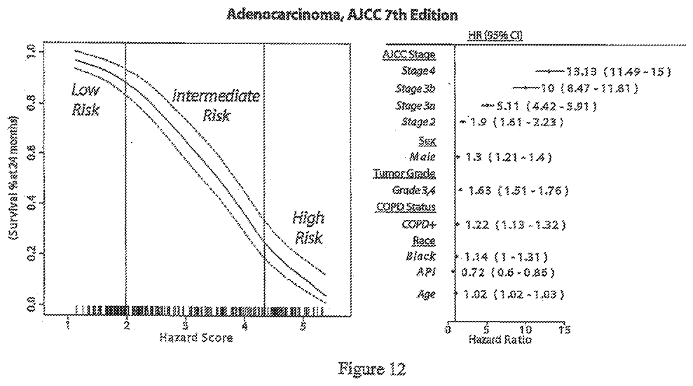

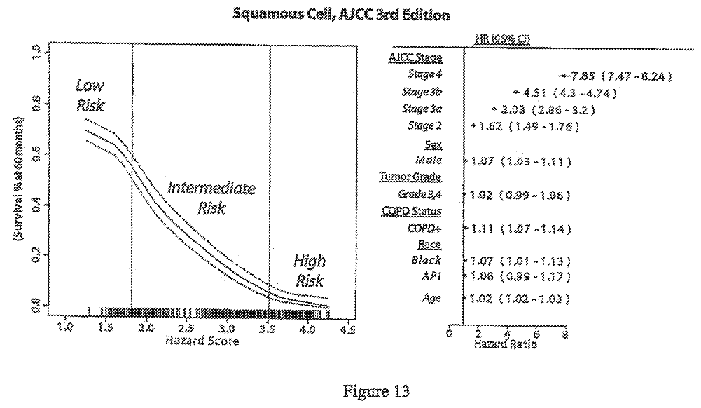

In the group of adenocarcinoma patients recoded to AJCC 7^{th} Edition staging, there was a significant difference in survival between patients with and without COPD in Stage I surgical patients treated without systemic therapy (p=0.0374). This same difference was also present in Stage 2 and 3a surgical patients treated without systemic therapy (p=0.0101). There was a similar trend in the group of Stage 2 and 3a patients treated with surgery and systemic therapy, but the difference in survival was not significant for any of the systemic therapy groups (p>0.05). The group of Stage 2 and 3a patients without COPD also experienced significantly better survival when treated with radiation without systemic therapy (p=0.0025, FIG. 12).

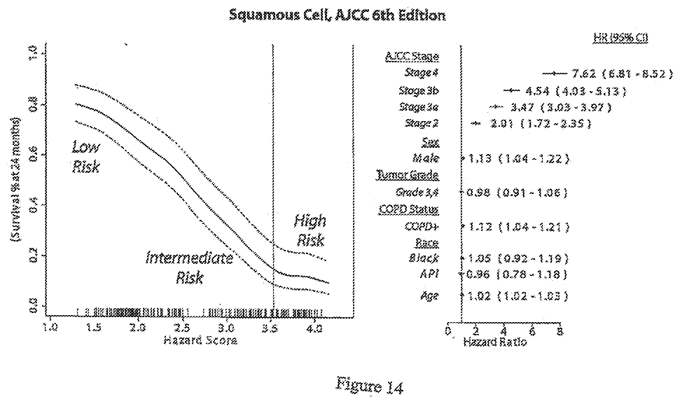

In squamous cell patients staged using the original 3^{rd} Edition the difference in survival when stratifying on COPD status for Stage I surgical patients treated without systemic therapy was significant, with patients having COPD again experiencing poorer survival (p=0.0002). There was also a significant stratification in the corresponding group of Stage I surgical patients treated with systemic therapy (p<0.05). COPD was able to produce a significant stratification in Stage 2 and 3a patients treated with systemic therapy in both the surgical and radiation groups (p<0.05, FIG. 13). In patients staged using the original 6^{th} Edition, COPD was able to produce a single stratification in Stage 2 and 3a surgical patients treated without systemic therapy (p=0.0091, FIG. 14).

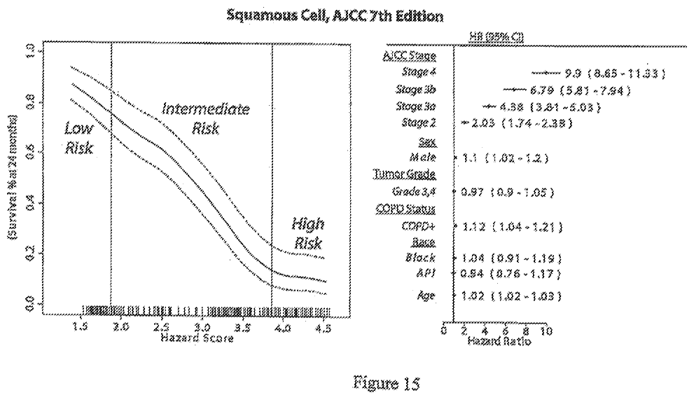

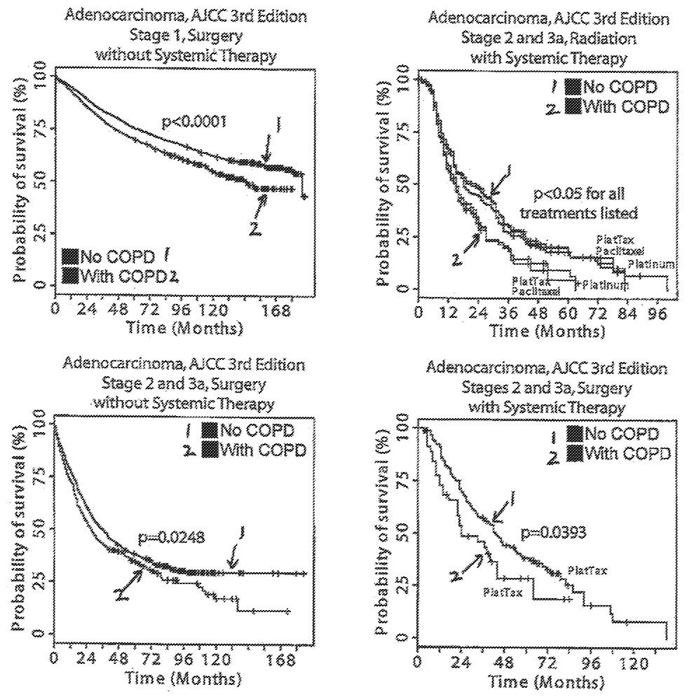

In the recoded 7^{th} Edition staging, there were two groups where COPD was able to stratify patients. The first was in Stage 2 and 3a patients treated with surgery without any systemic therapy, with COPD patients having poorer survival (p=0.0058, FIG. 15). The same pattern was observed in Stage 2 and 3a patients treated with radiation without any systemic therapy (p=0.0350, FIG. 13).

SUPPLEMENTARY TABLE 1
Result of modeling survival in a model with
each comorbid condition as an independent predictor. Shown are conditions
which confer significantly poorer survival in one or both of the histologies
considered when the sample was restricted to patients receiving
surgery without radiation or chemotherapy.

| | Adenocarcinoma | | Squamous Cell | |
|---|---|---|---|---|
| Condition | Odds Ratio | p-value | Odds Ratio | p-value |
| Congestive Heart | 1.37 | <0.0001* | 1.27 | 0.0003* |
| Failure | | | | |
| Peripheral Vascular | 1.22 | 0.0020* | 1.03 | 0.7234 |
| Disease | | | | |
| Cerebrovascular | 1.16 | 0.0152* | 1.06 | 0.3874 |
| Disease | | | | |
| COPD | 1.24 | <0.0001* | 1.11 | 0.0140* |
| Diabetes with sequelae | 1.26 | 0.0186* | 1.06 | 0.5964 |
| Chronic Renal Failure | 1.18 | 0.4083 | 1.57 | 0.0002* |
| Cirrhosis | 0.74 | 0.3000 | 1.95 | 0.0024* |
| Gastrointestinal Ulcers | 1.33 | 0.0193* | 1.22 | 0.1337 |

SUPPLEMENTARY TABLE 2
Methodology for assigning patients to
outcome groups based on survival time and status, for use in comparing
the prevalence of COPD in the AJCC 3^{rd} Edition staging scheme (A)
and AJCC 6^{th} Edition and recoded 7^{th} Edition (B). Survival status is based
on disease (lung and bronchus cancer) specific criteria.

| A. Survival Time/Status | Alive | Deceased |
|---|---|---|
| <24 Months | Intermediate | Short |
| >=60 Months | Long | Long |
| B. Survival Time/Status | Alive | Deceased |
| <12 Months | Intermediate | Short |
| >=24 Months | Long | Long |

SUPPLEMENTARY TABLE 3
Proportion of patients with COPD in long and
short-survival groups. A test of proportions was used to assess significant
differences in the prevalence of COPD in the two survival groups created
from patients treated with surgery without radiation or chemotherapy.

| | Cutoffs | | % COPD | | |
|---|---|---|---|---|---|
| Group | Short | Long | Short | Long | P-Value |
| Adenocarcinoma 3^{rd} | <24 mo | >=60 | 34.2% | 24.2% | <0.0001 |
| | | mo | | | |
| Adenocarcinoma 6^{th} | <12 mo | >=24 | 39.0% | 32.2% | 0.0438 |
| | | mo | | | |
| Adenocarcinoma 7^{th} | <12 mo | >=24 | 39.5% | 32.2% | 0.0326 |
| | | mo | | | |
| Squamous Cell 3^{rd} | <24 mo | >=60 | 39.9% | 34.0% | 0.0005 |
| | | mo | | | |
| Squamous Cell 6^{th} | <12 mo | >=24 | 53.5% | 44.9% | 0.0368 |
| | | mo | | | |
| Squamous Cell 7^{th} | <12 mo | >=24 | 53.6% | 44.8% | 0.0344 |
| | | mo | | | |

SUPPLEMENTARY TABLE 4
Mean AJCC tumor stage in long and short-
survival groups. A t-test was used to assess significant differences
in mean stage in the two survival groups created from patients treated
with surgery without radiation or chemotherapy.

| | Cutoffs | | Mean Stage | | |
|---|---|---|---|---|---|
| Group | Short | Long | Short | Long | P-Value |
| Adenocarcinoma 3^{rd} | <24 | >=60 mo | 2.03 | 1.17 | <0.0001 |
| | mo | | | | |
| Adenocarcinoma 6^{th} | <12 | >=24 mo | 2.22 | 1.29 | <0.0001 |
| | mo | | | | |
| Adenocarcinoma 7^{th} | <12 | >=24 mo | 2.26 | 1.42 | <0.0001 |
| | mo | | | | |
| Squamous Cell 3^{rd} | <24 | >=60 mo | 1.91 | 1.26 | <0.0001 |
| | mo | | | | |
| Squamous Cell 6^{th} | <12 | >=24 mo | 1.97 | 1.28 | <0.0001 |
| | mo | | | | |
| Squamous Cell 7^{th} | <12 | >=24 mo | 2.12 | 1.42 | <0.0001 |
| | mo | | | | |

SUPPLEMENTARY TABLE 5
Mean tumor grade in long and short-survival
groups. A t-test was used to assess significant differences in mean
tumor grade in the two survival groups created from patients treated
with surgery without radiation or chemotherapy.

| | Cutoffs | | Mean Grade | | |
|---|---|---|---|---|---|
| Group | Short | Long | Short Long | | P-Value |
| Adenocarcinoma 3^{rd} | <24 | >=60 mo | 2.46 | 2.08 | <0.0001 |
| | mo | | | | |
| Adenocarcinoma 6^{th} | <12 | >=24 mo | 2.38 | 2.03 | <0.0001 |
| | mo | | | | |
| Adenocarcinoma 7^{th} | <12 | >=24 mo | 2.40 | 2.03 | <0.0001 |
| | mo | | | | |
| Squamous Cell 3^{rd} | <24 | >=60 mo | 2.53 | 2.50 | 0.1486 |
| | mo | | | | |
| Squamous Cell 6^{th} | <12 | >=24 mo | 2.49 | 2.42 | 0.1374 |
| | mo | | | | |
| Squamous Cell 7^{th} | <12 | >=24 mo | 2.42 | 2.50 | 0.1201 |
| | mo | | | | |

SUPPLEMENTARY TABLE 6
Mean patient age in long and short-survival
groups. A t-test was used to assess significant differences in patient
age in the two survival groups created from patients treated with
surgery without radiation or chemotherapy.

| | Cutoffs | | Mean Age | | |
|---|---|---|---|---|---|
| Group | Short | Long | Short | Long | P-Value |
| Adenocarcinoma 3^{rd} | <24 | >=60 mo | 73.80 | 72.30 | <0.0001 |
| | mo | | | | |
| Adenocarcinoma 6^{th} | <12 | >=24 mo | 75.03 | 73.40 | 0.0029 |
| | mo | | | | |
| Adenocarcinoma 7^{th} | <12 | >=24 mo | 74.85 | 73.42 | 0.0084 |
| | mo | | | | |
| Squamous Cell 3^{rd} | <24 | >=60 mo | 73.85 | 72.13 | <0.0001 |
| | mo | | | | |
| Squamous Cell 6^{th} | <12 | >=24 mo | 74.80 | 73.34 | 0.001 |
| | mo | | | | |
| Squamous Cell 7^{th} | <12 | >=24 mo | 74.82 | 73.35 | 0.0101 |
| | mo | | | | |

SUPPLEMENTARY TABLE 7
Proportion of patients classified as API (top)
or Black (bottom) in long and short-survival groups. A test of
proportions was used to assess significant differences in the prevalence
of minority groups in the two survival groups created from
patients treated with surgery without radiation or chemotherapy.

| | Cutoffs | | Race (API) | | P- |
|---|---|---|---|---|---|
| Group | Short | Long | Short | Long | Value |
| Adenocarcinoma 3^{rd} | <24 | >=60 mo | 4.70% | 5.70% | 0.1445 |
| | mo | | | | |
| Adenocarcinoma 6^{th} | <12 | >=24 mo | 6.18% | 6.58% | 0.9219 |
| | mo | | | | |
| Adenocarcinoma 7^{th} | <12 | >=24 mo | 6.32% | 6.50% | 1 |
| | mo | | | | |
| Squamous Cell 3^{rd} | <24 | >=60 mo | 3.54% | 3.51% | 1 |
| | mo | | | | |
| Squamous Cell 6^{th} | <12 | >=24 mo | 3.98% | 2.95% | 0.6176 |
| | mo | | | | |
| Squamous Cell 7^{th} | <12 | >=24 mo | 4.00% | 3.00% | 0.606 |
| | mo | | | | |
| | Cutoffs | | Race (Black) | | P- |
| Group | Short | Long | Short | Long | Value |
| Adenocarcinoma 3^{rd} | <24 | >=60 mo | 6.40% | 5.10% | 0.0865 |
| | mo | | | | |
| Adenocarcinoma 6^{th} | <12 | >=24 mo | 6.18% | 4.96% | 0.5221 |
| | mo | | | | |
| Adenocarcinoma 7^{th} | <12 | >=24 mo | 6.32% | 4.97% | 0.4728 |
| | mo | | | | |
| Squamous Cell 3^{rd} | <24 | >=60 mo | 8.03% | 8.68% | 0.5516 |
| | mo | | | | |
| Squamous Cell 6^{th} | <12 | >=24 mo | 4.87% | 6.69% | 0.4324 |
| | mo | | | | |
| Squamous Cell 7^{th} | <12 | >=24 mo | 4.91% | 6.71% | 0.4448 |
| | mo | | | | |

SUPPLEMENTARY TABLE 8
Proportion of male patients in the long and short-
survival groups. A test of proportions was used to assess significant
differences in sex in the two survival groups created from patients
treated with surgery without radiation or chemotherapy.

| | Cutoffs | | Sex (Male) | | |
|---|---|---|---|---|---|
| Group | Short | Long | Short | Long | P-Value |
| Adenocarcinoma 3^{rd} | <24 | >=60 mo | 56.50% | 39.90% | <0.0001 |
| | mo | | | | |
| Adenocarcinoma 6^{th} | <12 | >=24 mo | 52.12% | 40.94% | 0.0014 |
| | mo | | | | |
| Adenocarcinoma 7^{th} | <12 | >=24 mo | 52.17% | 40.92% | 0.0014 |
| | mo | | | | |
| Squamous Cell 3^{rd} | <24 | >=60 mo | 68.31% | 59.08% | <0.0001 |
| | mo | | | | |
| Squamous Cell 6^{th} | <12 | >=24 mo | 64.16% | 55.71% | 0.03916 |
| | mo | | | | |
| Squamous Cell 7^{th} | <12 | >=24 mo | 64.29% | 55.62% | 0.0350 |
| | mo | | | | |

SUPPLEMENTARY TABLE 10
P-values estimated by comparing the Nagelkerke's R² statistic from 100 bootstrapped
samples using the Cox model generated on the entire patient sample for the
original Comprehensive model, and the same model estimated with a COPD indicator
added. Significant values are highlighted, with values showing degradation
in prognostication with the addition of COPD bolded and italicized.

| | | | | | |
|---|---|---|---|---|---|
| Total | No Chemo | Any Chemo | Platinum | Paclitaxel | Plat + Tax |

Adenocarcinoma 3rd

| | | | | | | |
|---|---|---|---|---|---|---|
| Any Treatment | 0.1584 | 0.1201 | 0.9228 | 0.9007 | 0.9004 | 0.8727 |
| Surgery Only | 0.0866 | 0.1202 | 0.8685 | 0.9079 | 0.8471 | 0.8309 |
| Radiation Only | 0.5638 | 0.7560 | 0.4240 | 0.4644 | 0.2454 | 0.5246 |
| Surg + Rad | 0.5914 | 0.6995 | 0.8545 | 0.8178 | 0.9995 | 0.8949 |
| No Treatment | 0.4754 | 0.1228 | 0.5587 | 0.5011 | 0.5585 | 0.2461 |

Adenocarcinoma 6th

| | | | | | | |
|---|---|---|---|---|---|---|
| Any Treatment | 0.2176 | 0.1477 | 0.6948 | 0.8449 | 0.9706 | 0.9137 |
| Surgery Only | 0.0874 | 0.2201 | 0.5911 | 0.5291 | 0.9314 | 0.6899 |
| Radiation Only | 0.1130 | 0.1663 | 0.8211 | 0.9646 | 0.5765 | 0.6126 |
| Surg + Rad | 0.4996 | 0.9854 | 0.2297 | 0.1906 | 0.9444 | 0.9301 |
| No Treatment | 0.3263 | 0.6722 | 0.7703 | 0.8269 | 0.4565 | 0.9344 |

Adenocarcinoma 7th

| | | | | | | |
|---|---|---|---|---|---|---|
| Any Treatment | 0.0814 | 0.3379 | 0.5324 | 0.6550 | 0.6325 | 0.5860 |
| Surgery Only | 0.0679 | 0.1815 | 0.5138 | 0.3998 | 0.9427 | 0.4235 |
| Radiation Only | 0.2211 | 0.3464 | 0.9340 | 0.7292 | 0.9713 | 0.3919 |
| Surg + Rad | 0.5132 | 0.9514 | 0.4026 | 0.4431 | 0.9588 | 0.8120 |
| No Treatment | 0.2814 | 0.5350 | 0.4753 | 0.6372 | 0.8534 | 0.6812 |

SUPPLEMENTARY TABLE 12
P-values estimated by comparing the Brier score at 36 months for the 3^{rd} Edition
and 24 months for the 6^{th} and recoded 7^{th} Edition from 100 bootstrapped samples
using the Cox model generated on the entire patient sample for the original
Comprehensive model, and the same model estimated with a COPD indicator added.
Significant values are highlighted, with values showing degradation in prognostication
with the addition of COPD bolded and italicized.

| | | | | | |
|---|---|---|---|---|---|
| Total | No Chemo | Any Chemo | Platinum | Paclitaxel | Plat + Tax |

Adenocarcinoma 3rd

| | | | | | | |
|---|---|---|---|---|---|---|
| Any Treatment | 0.0801 | 0.1161 | 0.6341 | 0.7535 | 0.6077 | 0.6832 |
| Surgery Only | 0.3738 | 0.3481 | 0.7197 | 0.7455 | 0.6505 | 0.7429 |
| Radiation Only | 0.7452 | 0.7439 | 0.8391 | 0.8165 | 0.7534 | 0.8005 |
| Surg + Rad | 0.9379 | 0.8855 | 0.8526 | 0.9518 | 0.9409 | 0.8444 |
| No Treatment | 0.8303 | 0.7250 | 0.9825 | 0.9604 | 0.9445 | 0.8964 |

Adenocarcinoma 6th

| | | | | | | |
|---|---|---|---|---|---|---|
| Any Treatment | 0.1731 | 0.1335 | 0.8574 | 0.7562 | 0.9032 | 0.6800 |
| Surgery Only | 0.6337 | 0.6007 | 0.8431 | 0.9083 | 0.9250 | 0.8183 |
| Radiation Only | 0.3224 | 0.2946 | 0.7990 | 0.8363 | 0.9905 | 0.7586 |
| Surg + Rad | 0.6245 | 0.6985 | 0.9679 | 0.9779 | 0.9738 | 0.7066 |
| No Treatment | 0.6505 | 0.6440 | 0.9686 | 0.7483 | 0.7406 | 0.9309 |

Adenocarcinoma 7th

| | | | | | | |
|---|---|---|---|---|---|---|
| Any Treatment | 0.1273 | 0.0677 | 0.9072 | 0.5518 | 0.9375 | 0.5797 |
| Surgery Only | 0.5045 | 0.3737 | 0.8554 | 0.9413 | 0.9563 | 0.8841 |
| Radiation Only | 0.3143 | 0.2295 | 0.8211 | 0.9512 | 0.8165 | 0.6344 |
| Surg + Rad | 0.8202 | 0.8252 | 0.9340 | 0.9979 | 0.9321 | 0.7037 |
| No Treatment | 0.5261 | 0.4674 | 0.8933 | 0.7485 | 0.7839 | 0.9535 |

### REFERENCES

1. Spira, A. and D. S. Ettinger, Multidisciplinary management of lung cancer. N. Engl. J. Med, 2004. 350(4): p. 379-392.
2. Goodgame, B., et al., Risk of recurrence of resected stage I non-small cell lung cancer in elderly patients as compared with younger patients. J. Thorac. Oncol, 2009. 4(11): p. 1370-1374.
3. Crino, L., et al., Early stage and locally advanced (non-metastatic) non-small-cell lung cancer: ESMO Clinical Practice Guidelines for diagnosis, treatment and follow-up. Ann. Oncol, 2010. 21 Suppl 5: p. v103-v115.
4. Byron, E. and M. Pinder-Schenck, Systemic and targeted therapies for early-stage lung cancer. Cancer Control, 2014. 21(1): p. 21-31.
5. Aguiar, P. N., Jr., et al., PD-L1 expression as a predictive biomarker in advanced non-small-cell lung cancer: updated survival data. Immunotherapy, 2017. 9(6): p. 499-506.
6. CM, J. S., et al., Immunotherapeutic strategies in non-small-cell lung cancer: the present and the future. Immunotherapy, 2017. 9(6): p. 507-520.
7. Kaufman, H. L., Rational Combination Immunotherapy: Understand the Biology. Cancer Immunol Res, 2017. 5(5): p. 355-356.
8. Lavin, Y., et al., Innate Immune Landscape in Early Lung Adenocarcinoma by Paired Single-Cell Analyses. Cell, 2017. 169(4): p. 750-765.e17.
9. Su, Z., et al., An investigation of biomarkers derived from legacy microarray data for their utility in the RNA-seq era. Genome Biol, 2014. 15(12): p. 523.
10. Hood, L., et al., Systems biology and new technologies enable predictive and preventative medicine. Science, 2004. 306(5696): p. 640-643.
11. Votavova, H., et al., Optimized protocol for gene expression analysis in formalin-fixed, paraffin-embedded tissue using real-time quantitative polymerase chain reaction. Diagn. Mol. Pathol, 2009. 18(3): p. 176-182.
12. Bosotti, R., et al., Cross platform microarray analysis for robust identification of differentially expressed genes. BMC. Bioinformatics, 2007. 8 Suppl 1: p. S5.
13. Shedden, K., et al., Gene expression-based survival prediction in lung adenocarcinoma: a multi-site, blinded validation study. Nat. Med, 2008. 14(8): p. 822-827.
14. Zhu, C. Q., et al., Prognostic and predictive gene signature for adjuvant chemotherapy in resected non-small-cell lung cancer. J Clin Oncol, 2010. 28(29): p. 4417-24.
15. Tang, H., et al., A 12-gene set predicts survival benefits from adjuvant chemotherapy in non-small cell lung cancer patients. Clin Cancer Res, 2013. 19(6): p. 1577-86.
16. Kratz, J. R., et al., A practical molecular assay to predict survival in resected non-squamous, non-small-cell lung cancer: development and international validation studies. Lancet, 2012. 379(9818): p. 823-832.
17. Chen, G., et al., Development and validation of a quantitative real-time polymerase chain reaction classifier for lung cancer prognosis. J Thorac Oncol, 2011. 6(9): p. 1481-7.
18. Guo, N. L., et al., Confirmation of gene expression-based prediction of survival in non-small cell lung cancer. Clin Cancer Res, 2008. 14(24): p. 8213-8220.
19. Wan, Y. W., et al., Hybrid models identified a 12-gene signature for lung cancer prognosis and chemoresponse prediction. PLoS ONE, 2010. 5(8).
20. Guo, N. L., et al., A novel network model identified a 13-gene lung cancer prognostic signature. Int. J. Comput. Biol. Drug Des, 2011. 4(1): p. 19-39.
21. Wan, Y. W., D. G. Beer, and N. L. Guo, Signaling pathway-based identification of extensive prognostic gene signatures for lung adenocarcinoma. Lung Cancer, 2012. 76(1): p. 98-105.
22. Livak, K. J. and T. D. Schmittgen, Analysis of relative gene expression data using real-time quantitative PCR and the 2(-Delta Delta C(T)) Method. Methods, 2001. 25(4): p. 402-408.
23. Lau, S. K., et al., Three gene prognostic classifier for early-stage non small-cell lung cancer. J Clin Oncol, 2007. 25(35): p. 5562-9.
24. Navab, R., et al., Prognostic gene-expression signature of carcinoma-associated fibroblasts in non-small cell lung cancer. Proc Natl Acad Sci USA, 2011. 108(17): p. 7160-5.
25. Chen, H. Y., et al., A five-gene signature and clinical outcome in non-small-cell lung cancer. N. Engl. J. Med, 2007. 356(1): p. 11-20.
26. Starczynowski, D. T., et al., TRAF6 is an amplified oncogene bridging the RAS and NF-kappaB pathways in human lung cancer. J. Clin. Invest, 2011. 121(10): p. 4095-4105.
27. Buchan, S. L., A. Rogel, and A. Al-Shamkhani, The immunobiology of CD27 and OX40 and their potential as targets for cancer immunotherapy. Blood, 2017.
28. Turaj, A. H., et al., Antibody Tumor Targeting Is Enhanced by CD27 Agonists through Myeloid Recruitment. Cancer Cell, 2017.
29. Bang, A., et al., Multicenter Evaluation of the Tolerability of Combined Treatment With PD-1 and CTLA-4 Immune Checkpoint Inhibitors and Palliative Radiation Therapy. Int J Radiat Oncol Biol Phys, 2017. 98(2): p. 344-351.
30. Lazzari, C., et al., SECOND-LINE THERAPY OF SQUAMOUS NON-SMALL CELL LUNG CANCER: AN EVOLVING LANDSCAPE. Expert Rev Respir Med, 2017.
31. Guo, L., et al., Constructing molecular classifiers for the accurate prognosis of lung adenocarcinoma. Clin. Cancer Res, 2006. 12(11): p. 3344-3354.
32. Ibraghimov-Beskrovnaya, O., et al., Primary structure of dystrophin-associated glycoproteins linking dystrophin to the extracellular matrix. Nature, 1992. 355(6362): p. 696-702.
33. Champelovier, P., et al., Dag-1 carcinoma cell in studying the mechanisms of progression and therapeutic resistance in bladder cancer. Eur Urol, 2001. 39(3): p. 343-8.
34. Yamamoto, H., T. Kishimoto, and S. Minamoto, NF-kappaB activation in CD27 signaling: involvement of TNF receptor-associated factors in its signaling and identification of functional region of CD27. J Immunol, 1998. 161(9): p. 4753-9.
35. Buchan, S. L., et al., PD-1 Blockade and CD27 Stimulation Activate Distinct Transcriptional Programs That Synergize for CD8(+) T-Cell-Driven Antitumor Immunity. Clin Cancer Res, 2018.
36. Forde, P. M., et al., Neoadjuvant PD-1 Blockade in Resectable Lung Cancer. N Engl J Med, 2018.
37. Mataki, C., et al., A novel zinc finger protein mRNA in human umbilical vein endothelial cells is profoundly induced by tumor necrosis factor alpha. J Atheroscler Thromb, 2000. 7(2): p. 97-103.
38. Kim, J. H., et al., A genome-wide association study of total serum and mite-specific IgEs in asthma patients. PLoS One, 2013. 8(8): p. e71958.
39. Pietila, T. E., et al., Multiple NF-kappaB and IFN regulatory factor family transcription factors regulate CCL19 gene expression in human monocyte-derived dendritic cells. J Immunol, 2007. 178(1): p. 253-61.
40. Kochel, T. J. and A. M. Fulton, Multiple drug resistance-associated protein 4 (MRP4), prostaglandin transporter (PGT), and 15-hydroxyprostaglandin dehydrogenase (15-PGDH) as determinants of PGE2 levels in cancer. Prostaglandins Other Lipid Mediat, 2015. 116-117: p. 99-103.
41. Sassi, Y., et al., Multidrug resistance-associated protein 4 regulates cAMP-dependent signaling pathways and controls human and rat SMC proliferation. J Clin Invest, 2008. 118(8): p. 2747-57.
42. Wen, J., et al., The Pharmacological and Physiological Role of Multidrug-Resistant Protein 4. J Pharmacol Exp Ther, 2015. 354(3): p. 358-75.
43. Delmotte, P., et al., Tumor necrosis factor alpha increases the expression of glycosyltransferases and sulfotransferases responsible for the biosynthesis of sialylated and/or sulfated Lewis x epitopes in the human bronchial mucosa. J Biol Chem, 2002. 277(1): p. 424-31.
44. Laubli, H., et al., L-selectin facilitation of metastasis involves temporal induction of Fut7-dependent ligands at sites of tumor cell arrest. Cancer Res, 2006. 66(3): p. 1536-42.
45. Liu, M. J., et al., ZIP8 regulates host defense through zinc-mediated inhibition of NF-kappaB. Cell Rep, 2013. 3(2): p. 386-400.
46. Chang, X., et al., Ligand-independent regulation of transforming growth factor beta1 expression and cell cycle progression by the aryl hydrocarbon receptor. Mol Cell Biol, 2007. 27(17): p. 6127-39.
47. Fang, F., et al., Early growth response 3 (Egr-3) is induced by transforming growth factor-beta and regulates fibrogenic responses. Am J Pathol, 2013. 183(4): p. 1197-1208.
48. Iranmanesh, S. M. and N. L. Guo, Integrated DNA Copy Number and Gene Expression Regulatory Network Analysis of Non-small Cell Lung Cancer Metastasis. Cancer Inform, 2014. 13(Suppl 5): p. 13-23.
49. Guo, N. L. et al. A Predictive 7-Gene Assay and Prognostic Protein Biomarkers for Non-small CellLung cancer, EBioMedicine 32, 102-110, doi:10.1016/j.ebiom.2018.05.025 (2018).
50. Putila, J & Guo, N. L. Combining COPD with Clinical, Pathological and Demographic Information Refines Prognosis and Treatment Response Prediction of Non-small Cell Lung Cancer, PLoS.ONE 9: e100994 (2014).
51. Putila, J., Remick, S. C., & Guo, N. L. Combining clinical, pathological, and demographic factors refines prognosis of lung cancer: a population-based study. PLoS ONE 6: e-17493 (2011).
52. Ghosh, A. K., A. I. Spriggs, and D. Y. Mason. 1985. ‘Immunocytochemical staining of T and B lymphocytes in serous effusions’, J Clin Pathol, 38: 608-12.
53. Shedden, K., J. M. Taylor, S. A. Enkemann, M. S. Tsao, T. J. Yeatman, W. L. Gerald, S. Eschrich, I. Jurisica, T. J. Giordano, D. E. Misek, A. C. Chang, C. Q. Zhu, D. Strumpf, S. Hanash, F. A. Shepherd, K. Ding, L. Seymour, K. Naoki, N. Pennell, B. Weir, R. Verhaak, C. Ladd-Acosta, T. Golub, M. Gruidl, A. Sharma, J. Szoke, M. Zakowski, V. Rusch, M. Kris, A. Viale, N. Motoi, W. Travis, B. Conley, V. E. Seshan, M. Meyerson, R. Kuick, K. K. Dobbin, T. Lively, J. W. Jacobson, and D. G. Beer. 2008. ‘Gene expression-based survival prediction in lung adenocarcinoma: a multi-site, blinded validation study’, Nat. Med, 14: 822-27.
54. Wan, Y. W., R. A. Raese, J. E. Fortney, C. Xiao, D. Luo, J. Cavendish, L. F. Gibson, V. Castranova, Y. Qian, and N. L. Guo. 2012. ‘A smoking-associated 7-gene signature for lung cancer diagnosis and prognosis’ Int j Oncol, 41: 1387-96.

It will be appreciated by those persons skilled in the art that changes could be made to embodiments of the present invention described herein without departing from the broad inventive concept thereof. It is understood, therefore, that this invention is not limited by any particular embodiments disclosed, but is intended to cover the modifications that are within the spirit and scope of the invention, as defined by the appended claims.

### SEQUENCE LISTING

A SEQUENCE LISTING in computer-readable form (.txt file) accompanies this application having SEQ ID NO:1 through SEQ ID NO:10. The computer-readable form (.txt file) of the SEQUENCE LISTING is incorporated by reference into this application. The SEQUENCE LISTING in computer-readable form (.txt file) is electronically submitted along with the electronic submission of this application.

1. A method of providing a treatment to a patient having non-small cell lung cancer comprising:
extracting total RNA from a formalin fixed and paraffin embedded tumor of a non-small cell lung cancer of a patient after the surgical resection;
generating complementary DNA (cDNA) of the extracted total RNA from said formalin fixed or paraffin embedded patient tumor;
quantifying of mRNA expression of 7 genes of ABCC4 (SEQ ID NO:1), CCL19 (SEQ ID NO:2), SLC39A8 (SEQ ID NO:3), CD27 (SEQ ID NO:4), FUT7 (SEQ ID NO:5), ZNF71 (SEQ ID NO:6); and DAG1 (SEQ ID NO:7) using qRT-PCR;
normalizing of the quantification of said 7 genes with the quantification of a control gene UBC (SEQ ID NO:8) or a housekeeping gene; and
utilizing said normalized 7 gene mRNA expression quantification to determine whether said patient will benefit from receiving adjuvant chemotherapy or not.

2. The method of claim 1 further comprising administering to said patient a therapeutically effective amount of one of the following adjuvant chemotherapies (a) cisplatin and Taxol (paclitaxel), (b) cisplatin and Taxotere (docetaxel), (c) carboplatin, (d) carboplatin and Taxol (paclitaxel), (e) carboplatin and Taxotere (docetaxel), (f) Taxol (paclitaxel), and (g) Alimta (pemetrexed).

3. The method of claim 2 wherein said quantification of mRNA expression of three genes of ABCC4 (SEQ ID NO:1), CCL19 (SEQ ID NO:2), and SLC39A8 (SEQ ID NO:3) within said 7-genes.

4. The method of claim 2 wherein said quantification of mRNA expression of four genes of CD27 (SEQ ID NO:4), FUT7 (SEQ ID NO:5), ZNF71 (SEQ ID NO:6), and DAG1 (SEQ ID NO:7) within said 7-genes.

5. The method of claim 1 wherein said quantification of mRNA expression of ABCC4 (SEQ ID NO:1) and utilization of said normalized ABCC4 gene mRNA expression quantification to determine whether said patient will benefit from receiving adjuvant chemotherapy of one of (a) cisplatin and Taxol (paclitaxel), (b) cisplatin and Taxotere (docetaxel), (d) carboplatin and Taxol (paclitaxel), (e) carboplatin and Taxotere (docetaxel), and (f) Taxol (paclitaxel).

6. The method of claim 1 wherein said quantification of mRNA expression of FUT7 (SEQ ID NO:5) and utilization of said normalized FUT7 gene mRNA expression quantification to determine whether said patient will benefit from receiving adjuvant chemotherapy of carboplatin.

7. The method of claim 1 wherein said quantification of mRNA expression of ZNF71 (SEQ ID NO:6) and utilization of said normalized ZNF71 gene mRNA expression quantification to determine whether said patient will benefit from receiving adjuvant chemotherapy of (a) carboplatin and Taxol (paclitaxel), (b) carboplatin and Taxotere (docetaxel), (c) cisplatin and Taxotere (docetaxel), and (d) cisplatin and Taxol (paclitaxel).

8. The method of claim 1 wherein said quantification of mRNA expression of SLC39A8 (SEQ ID NO:3) and utilization of said normalized SLC39A8 gene mRNA expression quantification to determine whether said patient will benefit from receiving adjuvant chemotherapy of one of (a) Taxol (paclitaxel), and (b) Alimta (pemetrexed).

9. A method of providing a treatment to a patient having non-small cell lung cancer comprising:
providing protein expression of ZNF71 (SEQ ID NO: 9);
quantifying said protein expression of said ZNF71 with automated quantitative analysis (AQUA) correlated with said ZNF71 mRNA expression in a formalin fixed and paraffin embedded patient tumor using immunohistochemistry staining; and
determining a prognosis of said patient from said protein expression of said ZNF71.

10. The method of claim 9 including wherein said prognosis of said patient is either longer survival or shorter survival.

11. The method of claim 9 including combining one or more of the group selected from existence of patient COPD, patient age, patient gender, patient race, histology, AJCC staging edition, cancer stage, and tumor grade, using multivariate Cox model with SEER-Medicare data or a web-based model in determining said prognosis of said patient.

12. A method of providing a treatment to a patient having non-small cell lung cancer comprising:
providing protein expression of CD27 (SEQ ID NO:10);
quantifying said protein expression of said CD27 with ELISA correlated with said CD27 mRNA expression in a formalin fixed and paraffin embedded patient tumor using immunocytochemistry staining and a cancer-free tissue adjacent to said tumor; and
determining a prognosis of said patient from said protein expression of said CD27.

13. The method of claim 12 including wherein said prognosis of said patient is either longer survival or shorter survival.

14. The method of claim 12 including administering to said patient a therapeutically effective amount of an adjuvant chemotherapy.

15. The method of claim 12 including combining one or more of the group selected from existence of patient COPD, patient age, patient gender, patient race, histology, AJCC staging edition, cancer stage, and tumor grade, using multivariate Cox model with SEER-Medicare data or a web-based model in determining said prognosis of said patient.
